(19) United States
(12) Patent Application Publication
(10) Pub. No.: US 2025/0275991 A1
(43) Pub. Date: Sep. 4, 2025

(54) ORAL COMPLEMENT FACTOR D INHIBITORS

(71) Applicant: BioCryst Pharmaceuticals, Inc, Durham, NC (US)

(72) Inventors: Pravin L. Kotian, Hoover, AL (US); Yarlagadda S. Babu, Birmingham, AL (US); Weihe Zhang, Vestavia Hills, AL (US); Peng-Cheng LU, Vestavia Hills, AL (US); Andrew E. Spaulding, Hoover, AL (US); Minwan Wu, Vestavia Hills, AL (US); Wei Lv, Hoover, AL (US); Trung Xuan Nguyen, Hoover, AL (US); Zhao Dang, Vestavia Hills, AL (US); Krishnan Raman, Birmingham, AL (US)

(21) Appl. No.: 17/767,798

(22) PCT Filed: Oct. 9, 2020
(86) PCT No.: PCT/US20/54922

§ 371 Date: Apr. 8, 2022

Related U.S. Application Data
(60) Provisional application No. 62/913,021, filed on Oct. 9, 2019.

(51) Int. Cl. A61K 31/665; A61K 31/343; A61K 31/381; A61K 31/4025; A61K 31/404; A61K 31/4155; A61K 31/4178; A61K 31/422; A61K 31/427; A61K 31/443; A61K 31/4439; A61K 31/53; C07D 209/08; C07D 307/80; C07D 307/81; C07D 307/82; C07D 401/04; C07D 405/04; C07D 405/12; C07D 409/04; C07D 413/04; C07D 417/04; C07D 487/04; C07F 9/655

(52) U.S. Cl. CPC A61K 31/665; A61K 31/343; A61K 31/381; A61K 31/4025; A61K 31/404; A61K 31/4155; A61K 31/4178; A61K 31/422; A61K 31/427; A61K 31/443; A61K 31/4439; A61K 31/53; C07D 209/08; C07D 307/80; C07D 307/81; C07D 307/82; C07D 401/04; C07D 405/04; C07D 405/12; C07D 409/04; C07D 413/04; C07D 417/04; C07D 487/04; C07F 9/65517

(57) ABSTRACT

Disclosed are compounds of formula (I)-(IV), and pharmaceutically acceptable salts thereof, which are inhibitors of the complement system. Also provided are pharmaceutical compositions comprising such a compound, and methods of using the compounds and compositions in the treatment or prevention of a disease or condition characterized by aberrant complement system activity.

### RELATED APPLICATION

This application claims the benefit of priority to U.S. Provisional Patent Application No. 62/913,021, filed Oct. 9, 2019.

### BACKGROUND OF THE INVENTION

The complement system is a branch of an organism's immune system that enhances the ability of antibodies and phagocytic cells to destroy and remove foreign particles (e.g., pathogens) from the organism. The complement system comprises a set of plasma proteins that act together to attack extracellular forms of pathogens and induce a series of inflammatory responses to help fight infection. Complement activation can occur through several pathways. For example, complement activation can occur spontaneously in response to certain pathogens or by antibody binding to a pathogen. When complement proteins are activated a cascade is triggered by which one complement protein induces the activation of the next protein in the sequence. The activation of a small number of complement proteins at the start of the pathway is hugely amplified by each successive enzymatic reaction, resulting in the rapid generation of a disproportionately large complement response. (Marrides, S. Pharmacological Reviews 1998, Vol. 50, pages 59-88). In healthy organisms there are regulatory mechanisms to prevent uncontrolled complement activation.

When activated, complement proteins can bind to a pathogen, opsonizing them for engulfment by phagocytes bearing receptors for complement. Then, small fragments of some complement proteins act as chemoattractants to recruit more phagocytes to the site of complement activation, and also to activate these phagocytes. Next, the complement proteins create holes or pores in the invading organisms, leading to their destruction. While complement plays an important role in protecting the body from foreign organisms, it can also destroy healthy cells and tissue. The inappropriate activation of complement is implicated in a long list of disease pathologies (Morgan, B. Eur J Clin Invest 1994, Vol. 24, pages 219-228) affecting the immune, renal, cardiovascular, and neurological systems. Accordingly, there exists a need to develop further complement inhibitors, which have therapeutic potential in the treatment of numerous disorders.

### SUMMARY OF THE INVENTION

In certain aspects, the invention provides compounds having the structure of formula (I), and pharmaceutically acceptable salts thereof:

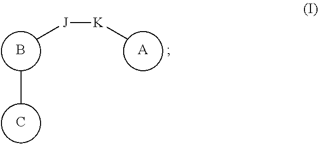

wherein:
\textcircled{A} is selected from the group consisting of

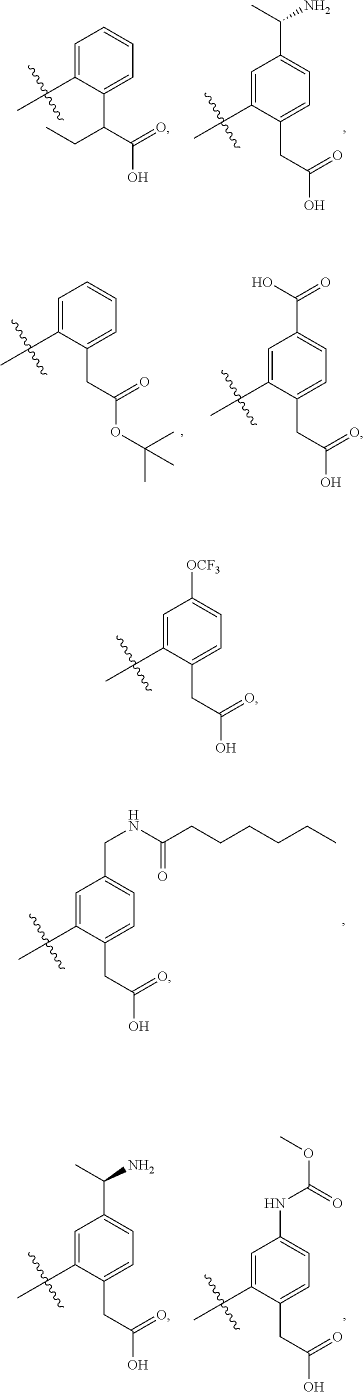

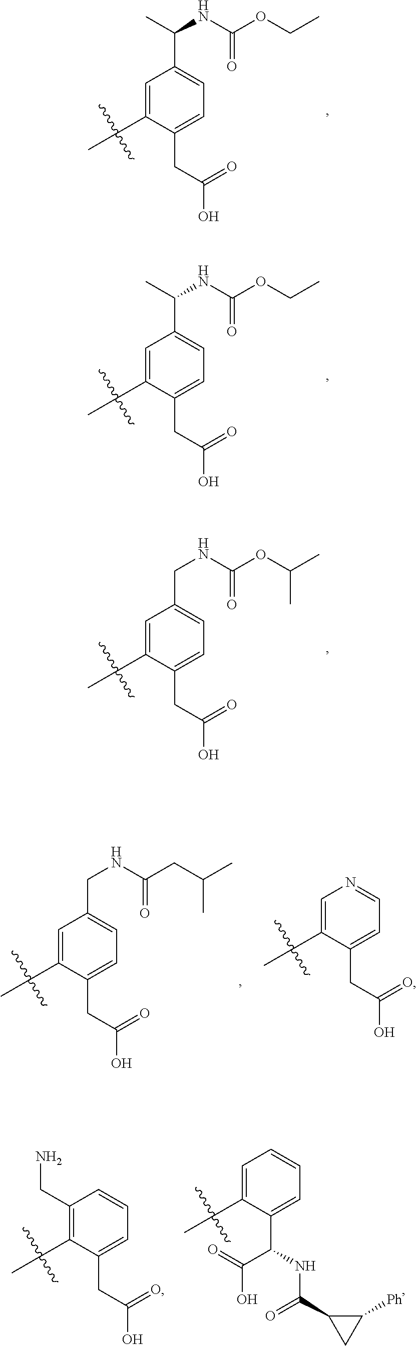

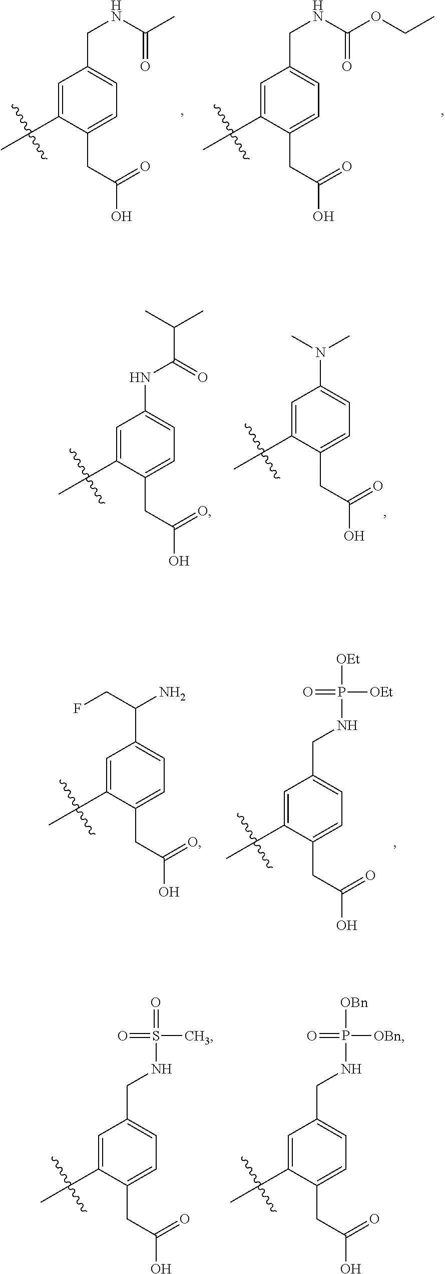

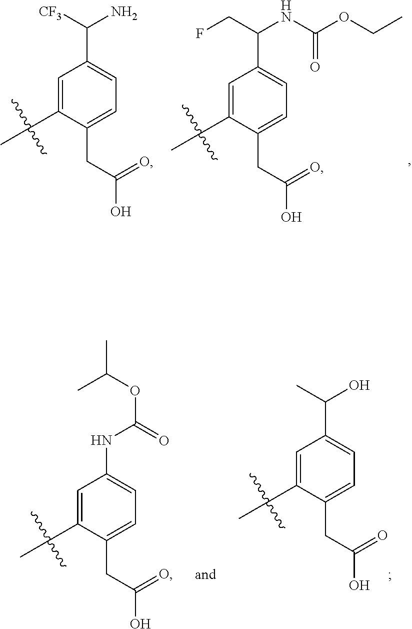 [SMILES: *c1cc(C(N)C(F)(F)F)ccc1CC(=O)O ; *c1cc(C(CF)NC(=O)OCC)ccc1CC(=O)O ; *c1cc(NC(=O)OC(C)C)ccc1CC(=O)O ; *c1cc(C(C)O)ccc1CC(=O)O]

\textcircled{B} is selected from the group consisting of

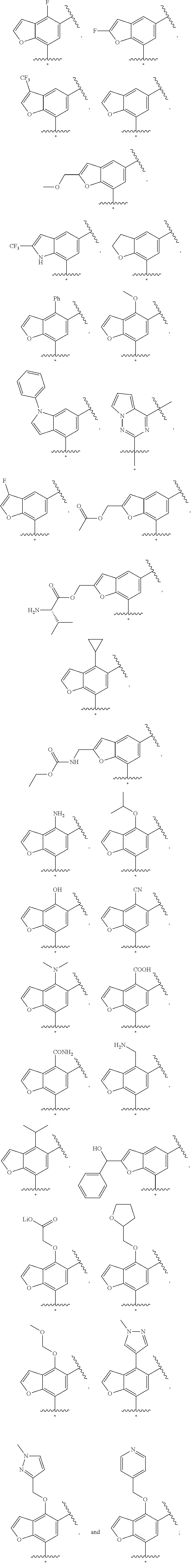

wherein the asterisk (*) indicates the point of attachment to \textcircled{C};

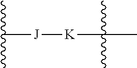

is selected from the group consisting of

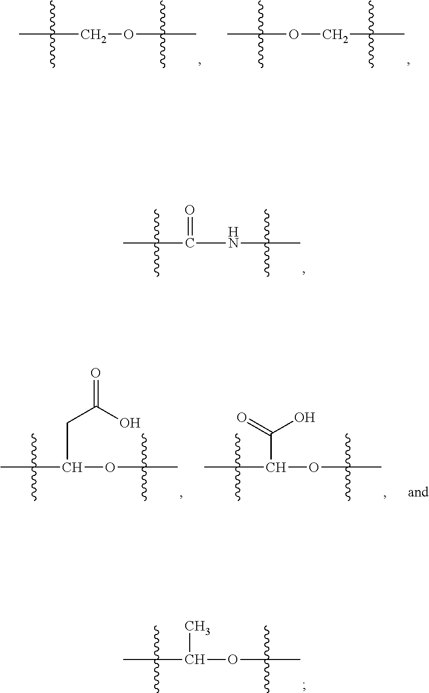

and
\textcircled{C} is selected from the group consisting of

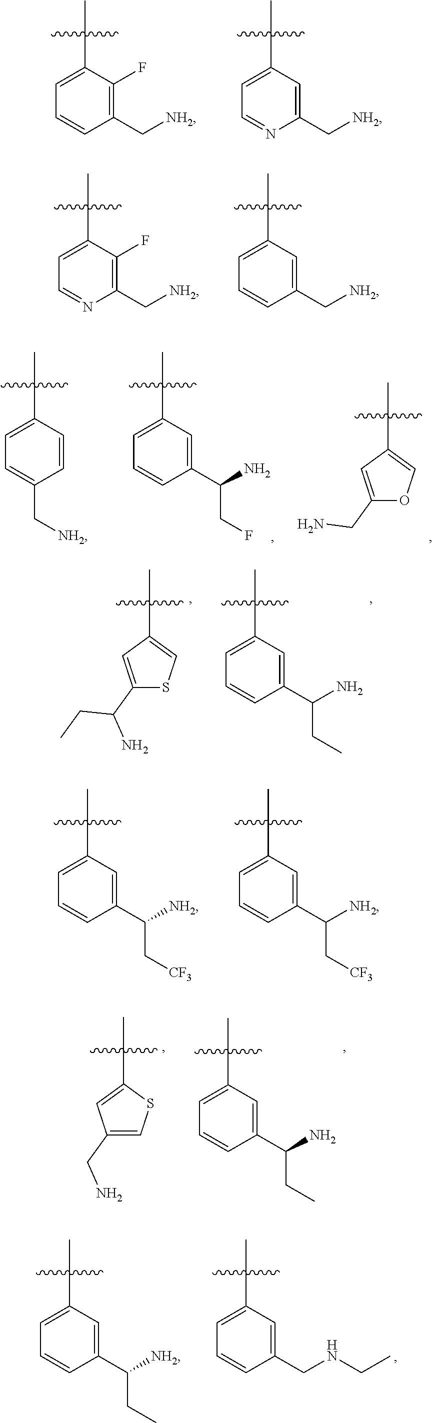

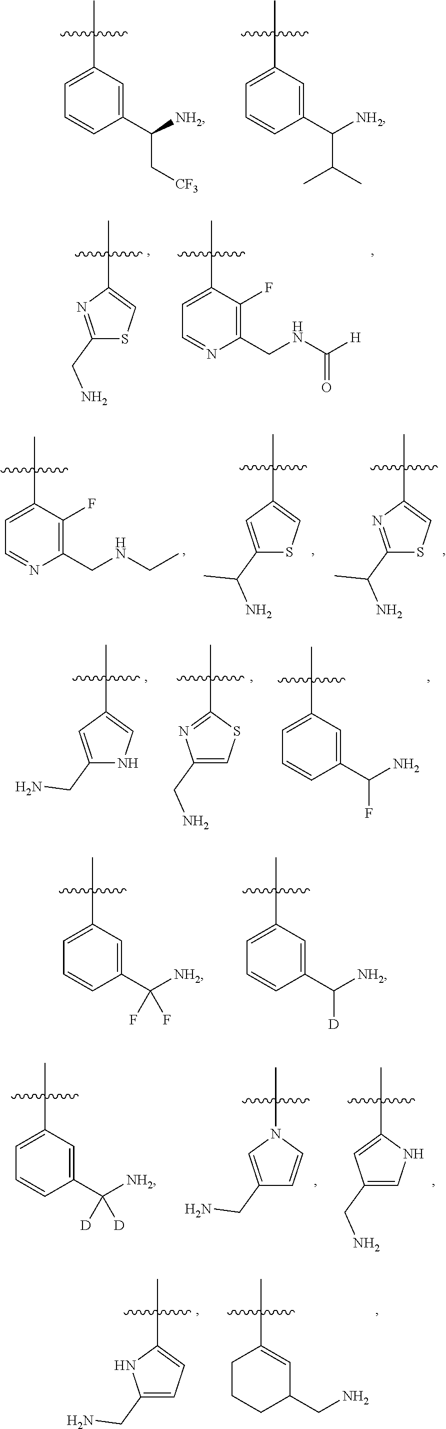

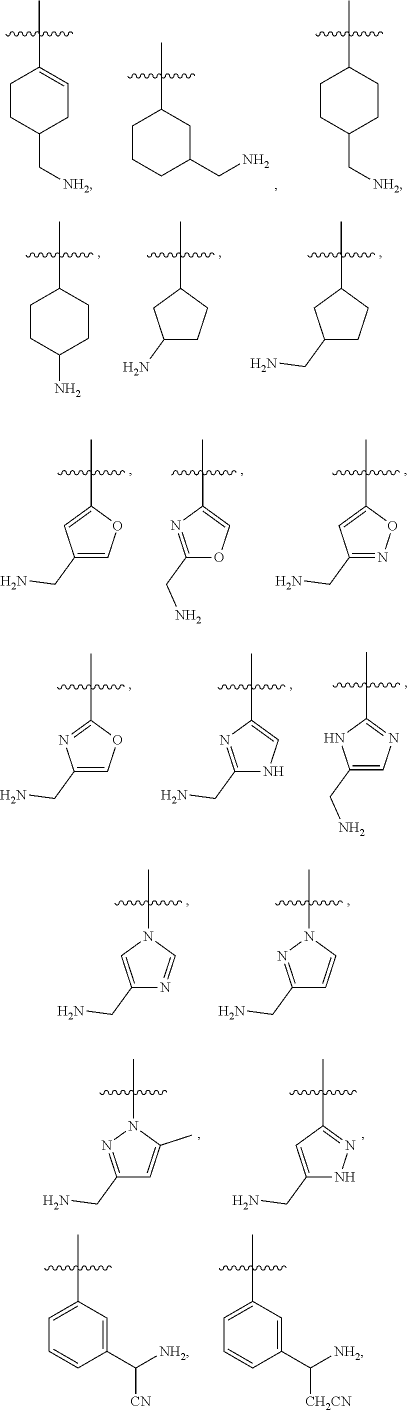

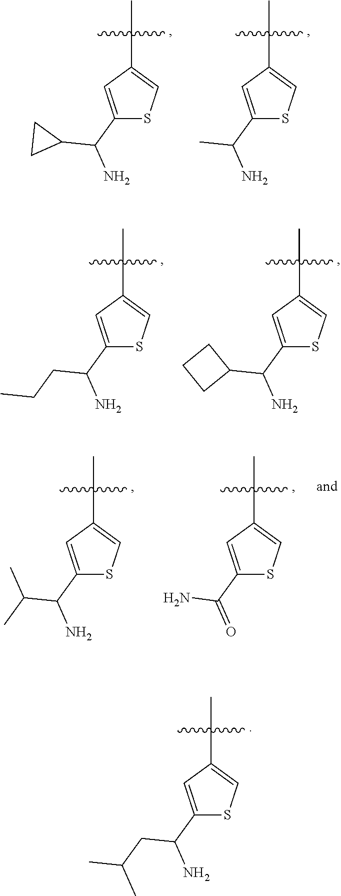

In further embodiments, the invention provides compounds of formula (II), and pharmaceutically acceptable salts thereof:

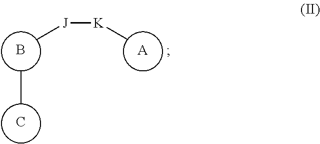

wherein:
\textcircled{A} is selected from the group consisting of

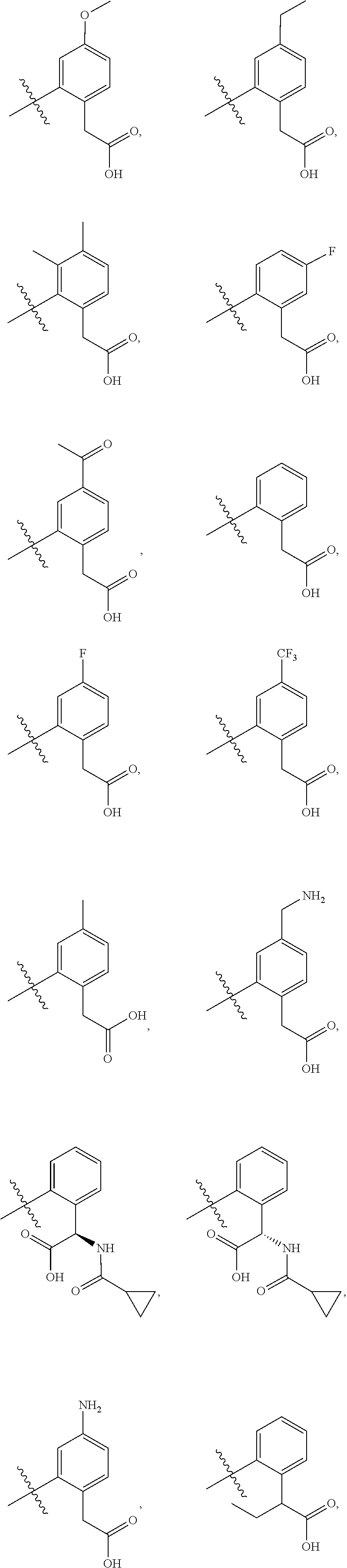

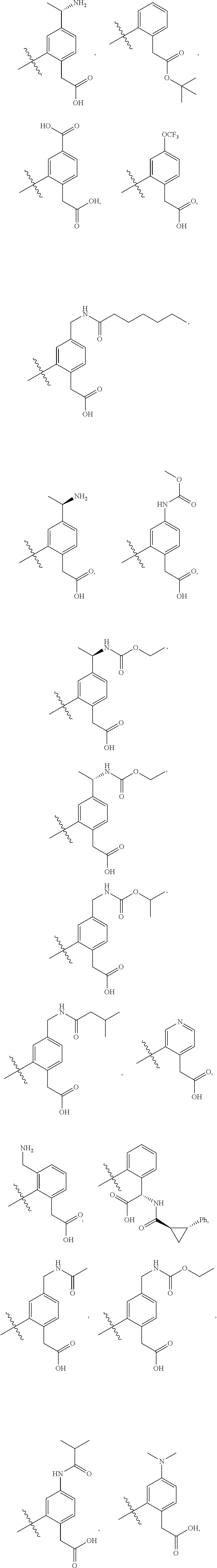

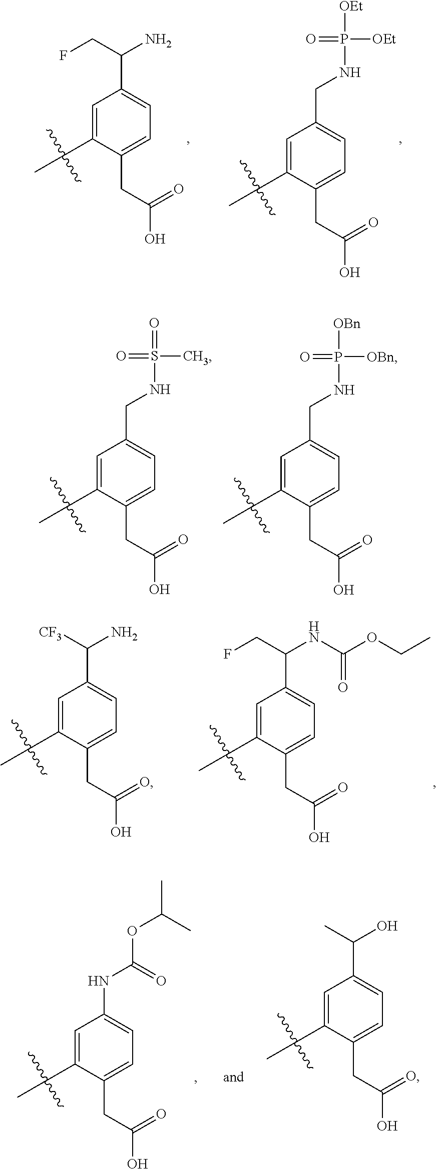

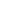

B is selected from the group consisting of

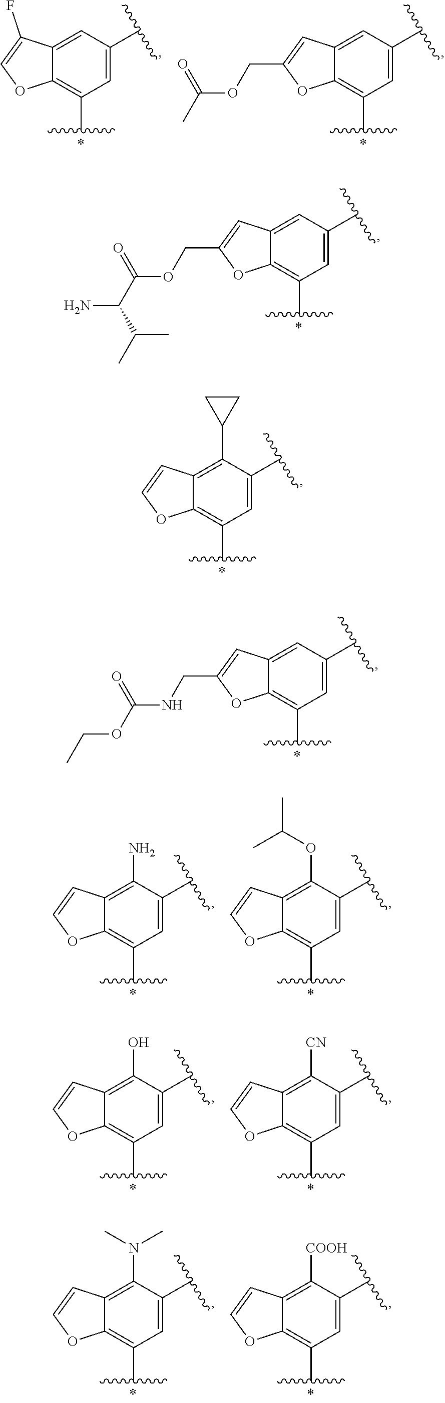

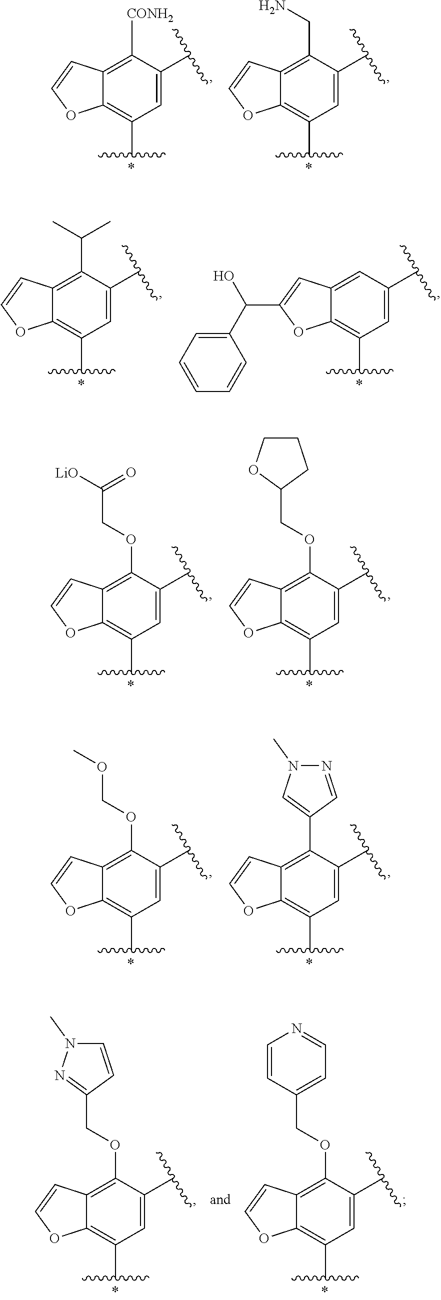

wherein the asterisk (*) indicates the point of attachment to \textcircled{C};

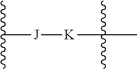

is selected from the group consisting of

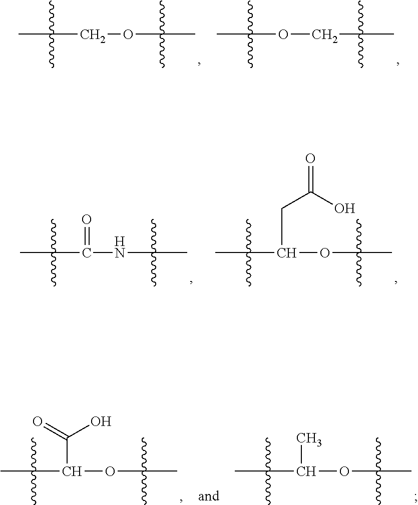

and
\textcircled{C} is selected from the group consisting of

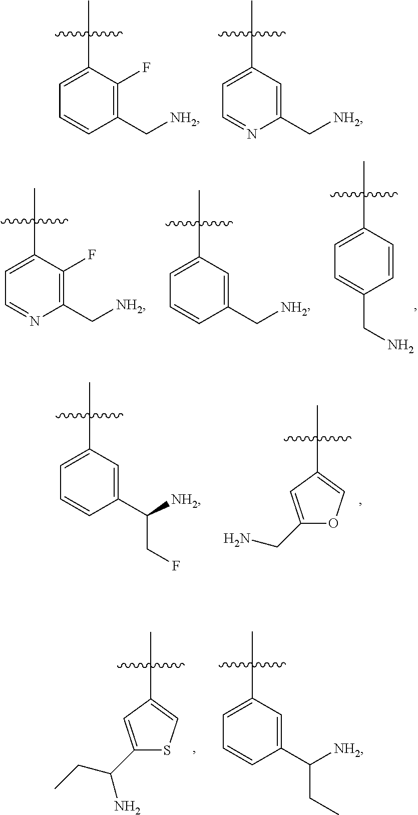

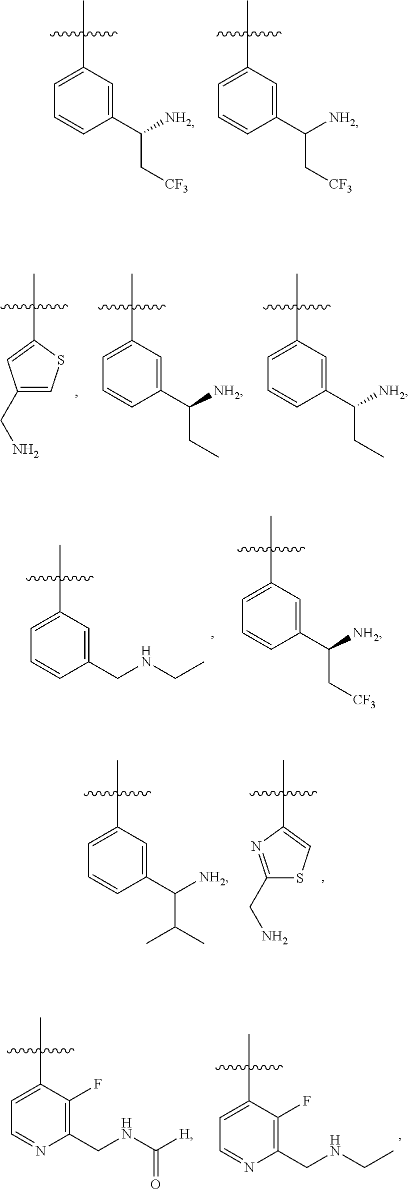

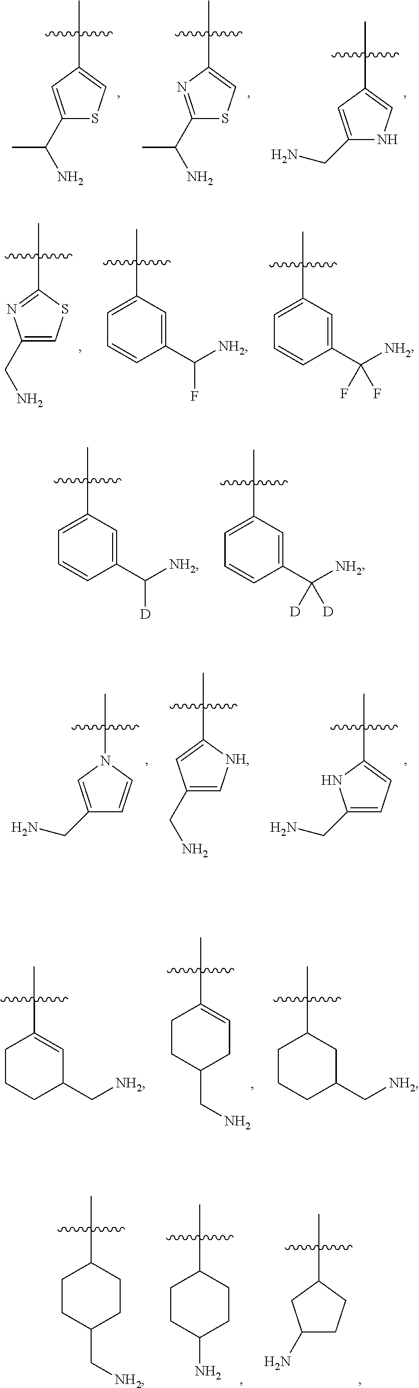

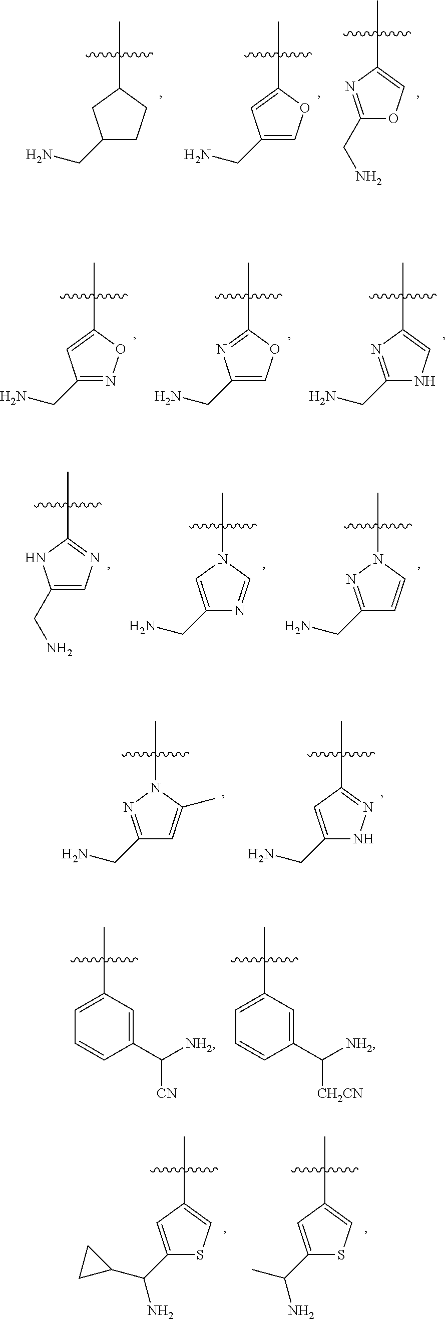

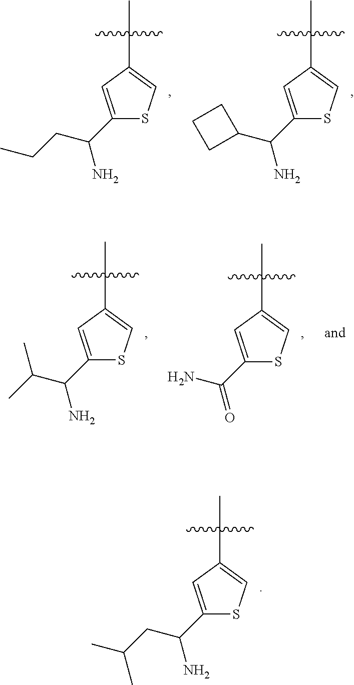 [SMILES: *c1csc(C(N)CCC)c1 ; *c1csc(C(N)C2CCC2)c1 ; *c1csc(C(N)C(C)C)c1 ; *c1csc(C(N)=O)c1 ; *c1csc(C(N)CC(C)C)c1]

In further embodiments, the invention provides compounds of formula (III), and pharmaceutically acceptable salts thereof:

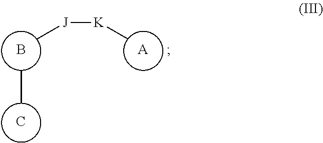

wherein:
\textcircled{A} is selected from the group consisting of

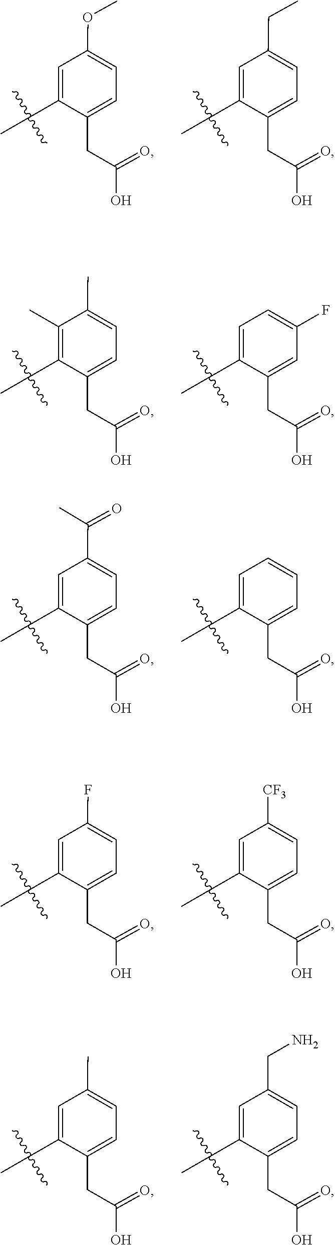

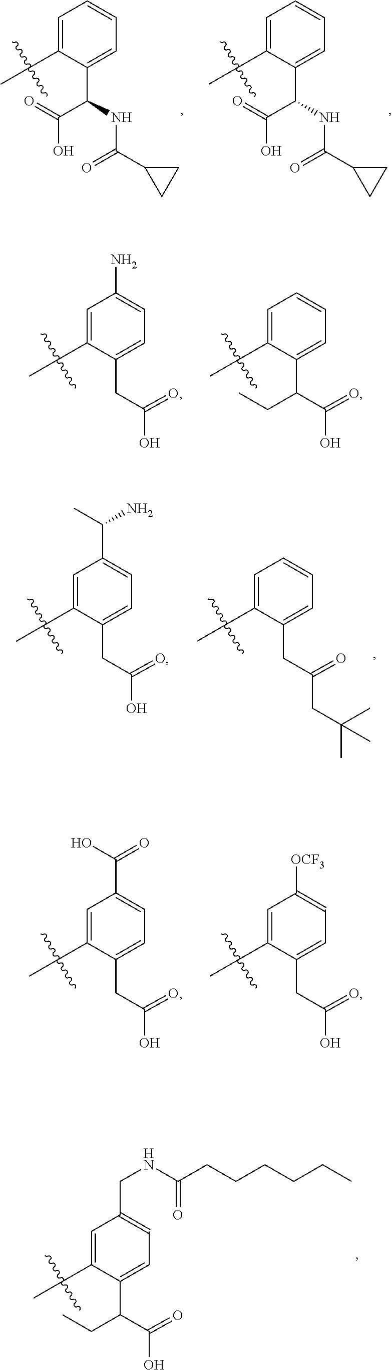

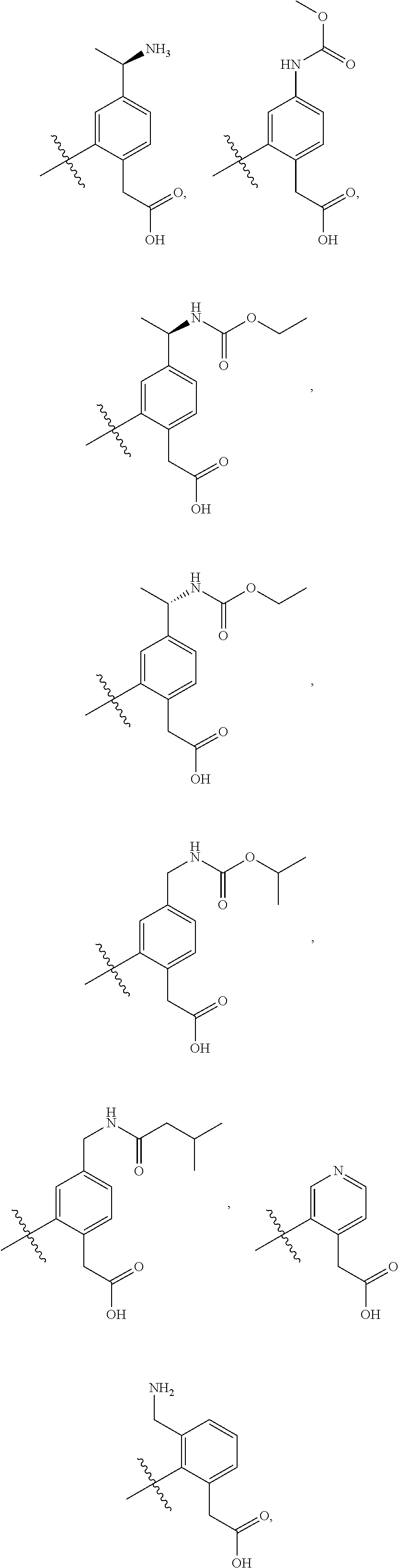

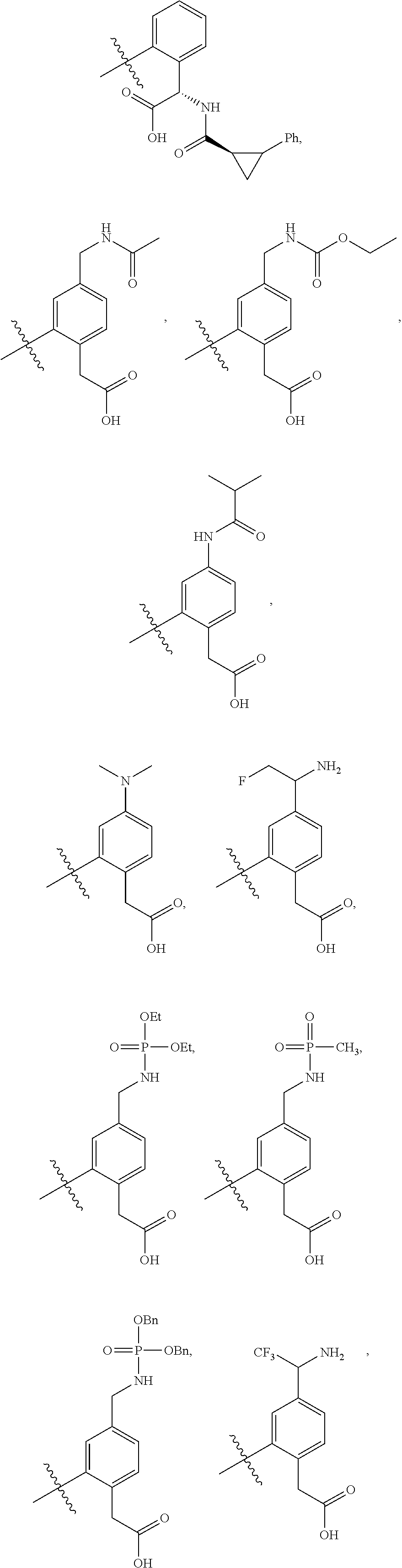

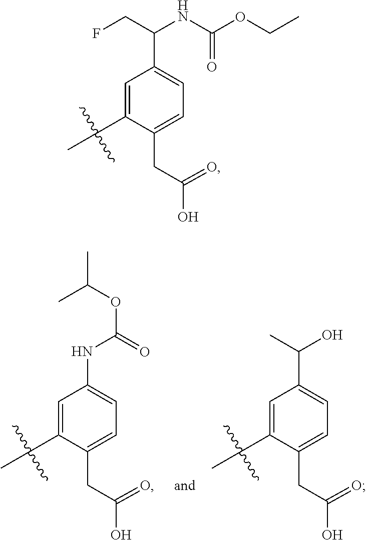 [SMILES: *c1cc(C(CF)NC(=O)OCC)ccc1CC(=O)O ; *c1cc(NC(=O)OC(C)C)ccc1CC(=O)O ; "and" ; *c1cc(C(C)O)ccc1CC(=O)O]

\textcircled{B} is selected from the group consisting of

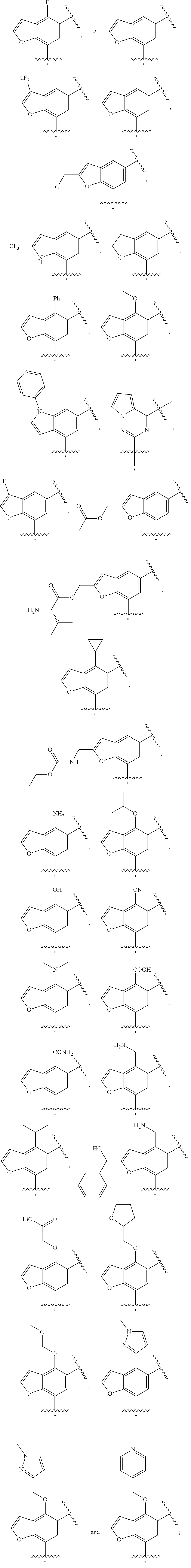

wherein the asterisk (*) indicates the point of attachment to \textcircled{C};

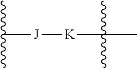

is selected from the group consisting of

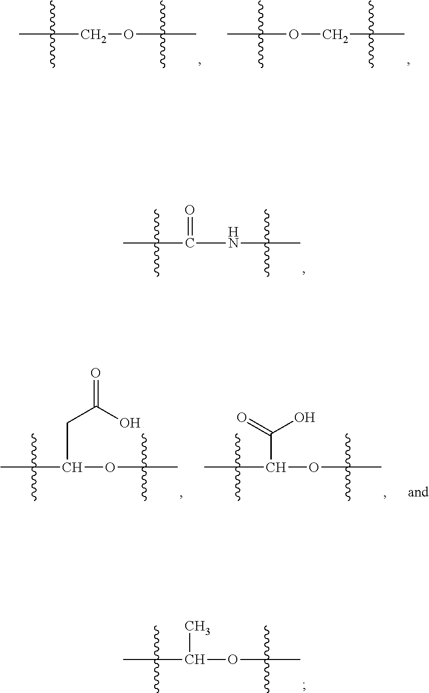

and
\textcircled{C} is selected from the group consisting of

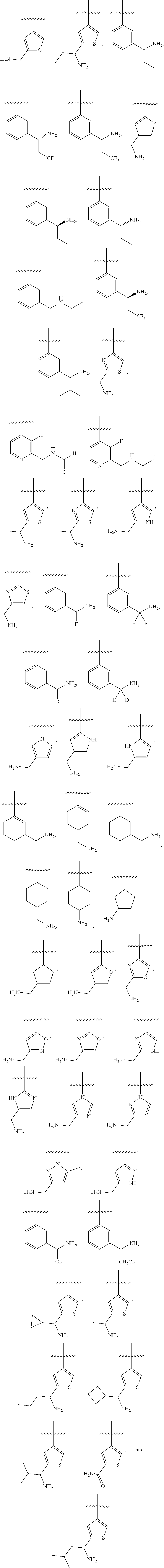

In further embodiments, the invention provides compounds of formula (IV), and pharmaceutically acceptable salts thereof:

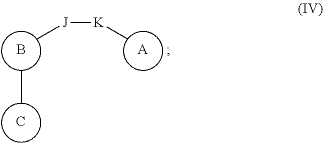

wherein:
\textcircled{A} is selected from the group consisting of

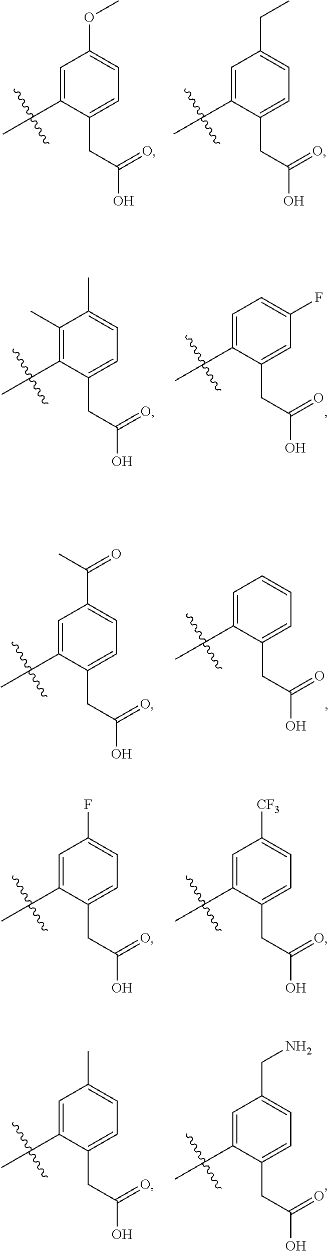

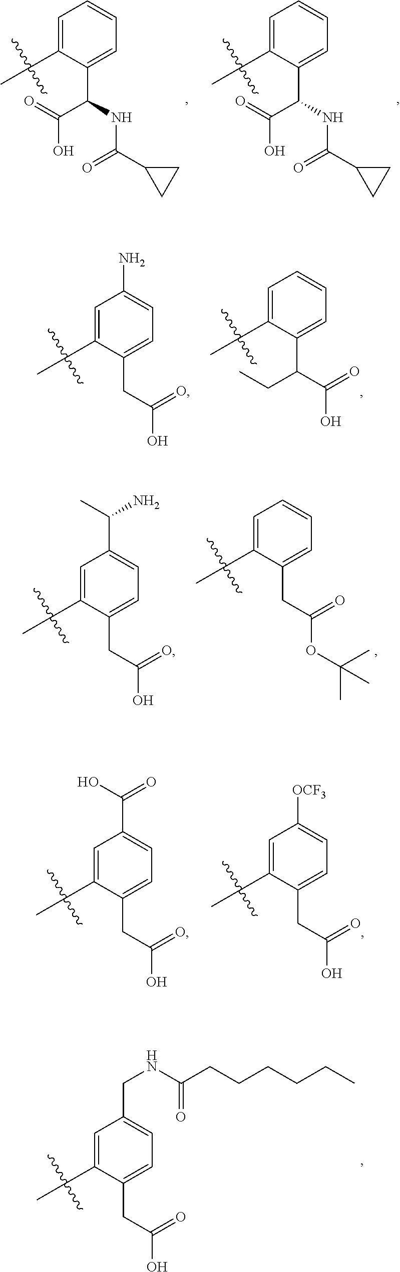

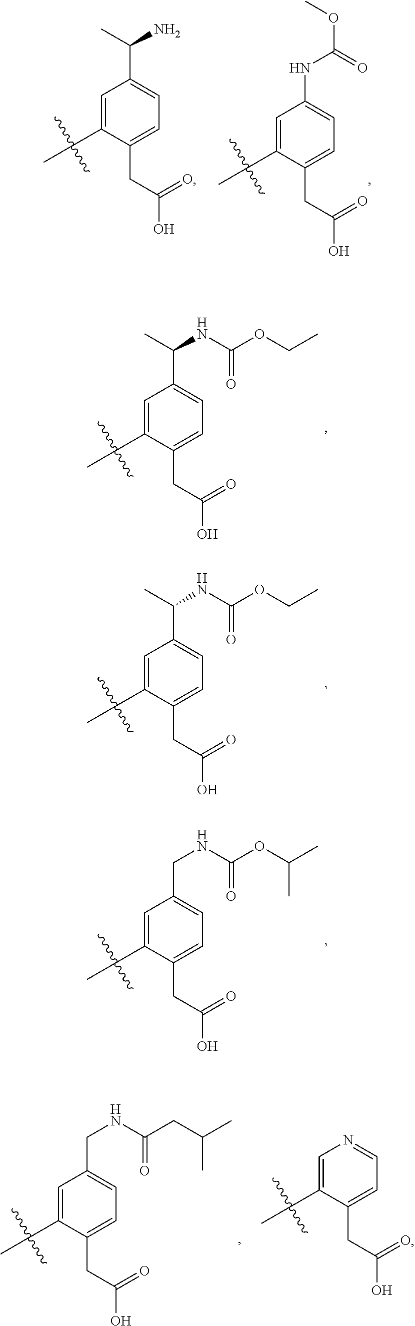

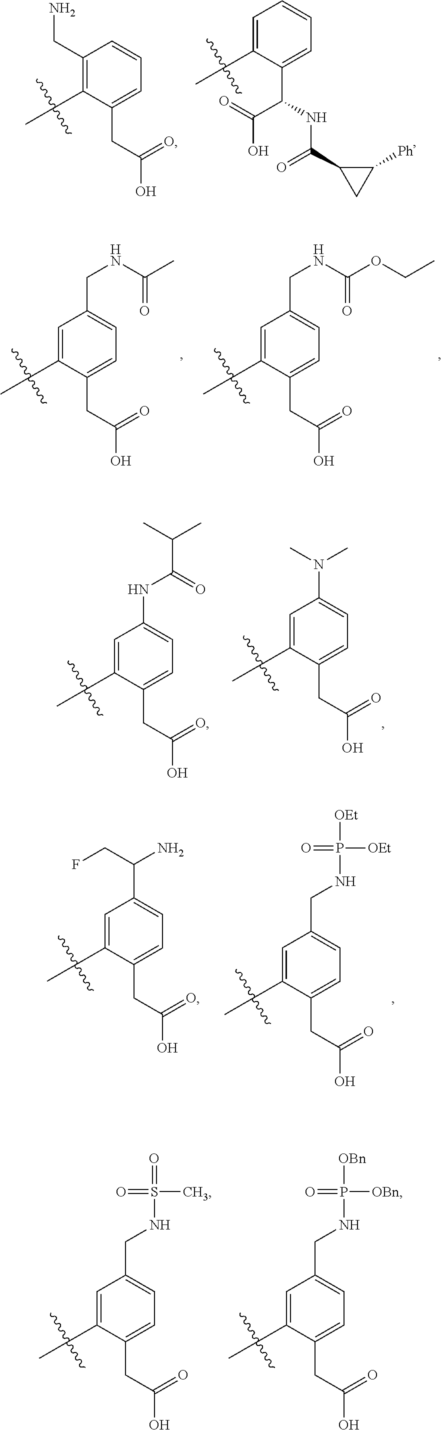

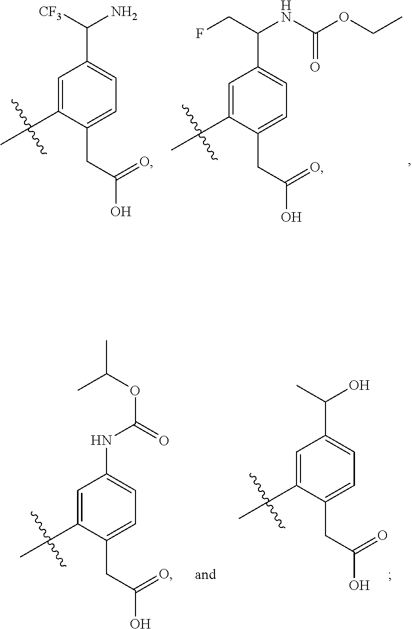 [SMILES: *c1cc(C(N)C(F)(F)F)ccc1CC(=O)O ; *c1cc(C(CF)NC(=O)OCC)ccc1CC(=O)O ; *c1cc(NC(=O)OC(C)C)ccc1CC(=O)O ; *c1cc(C(C)O)ccc1CC(=O)O]

\textcircled{B} is selected from the group consisting of

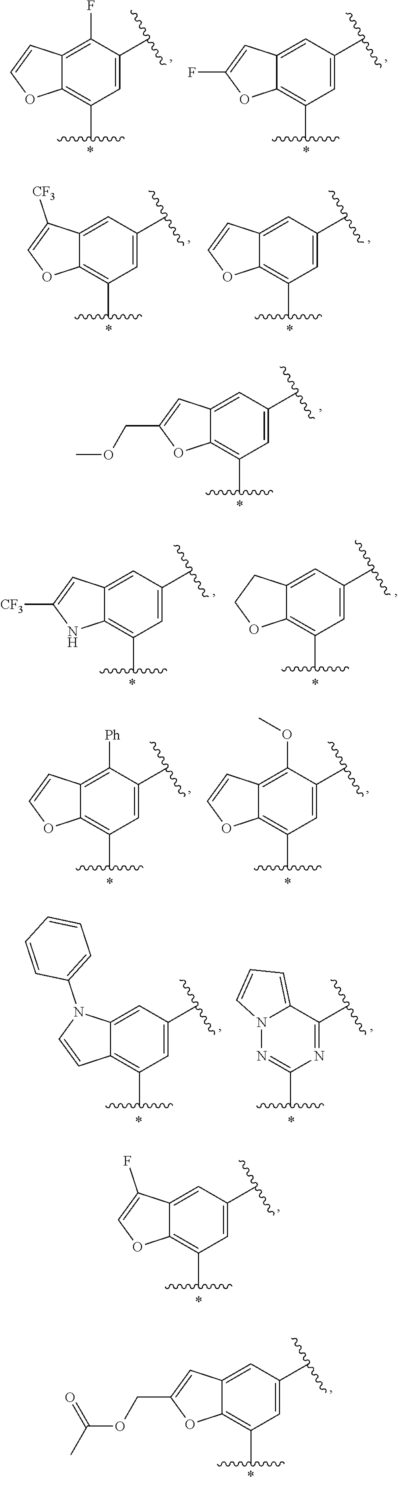

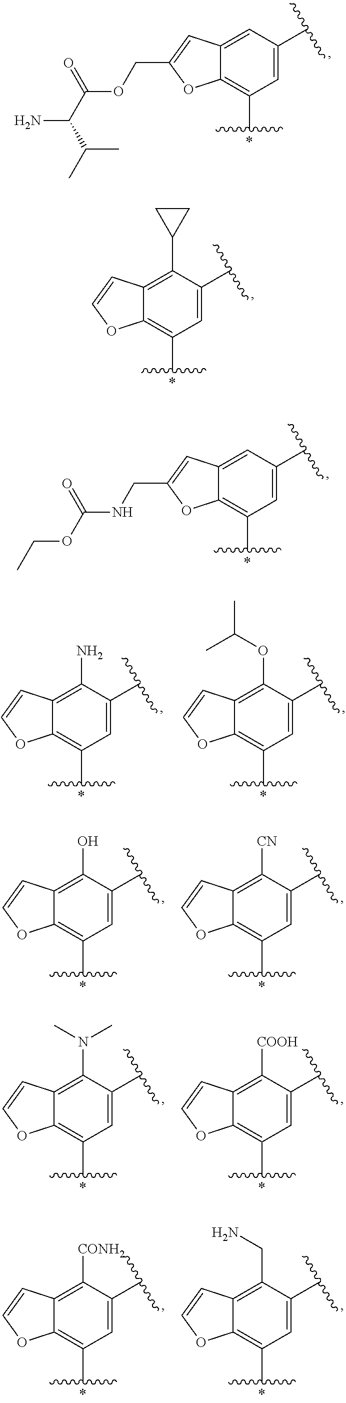

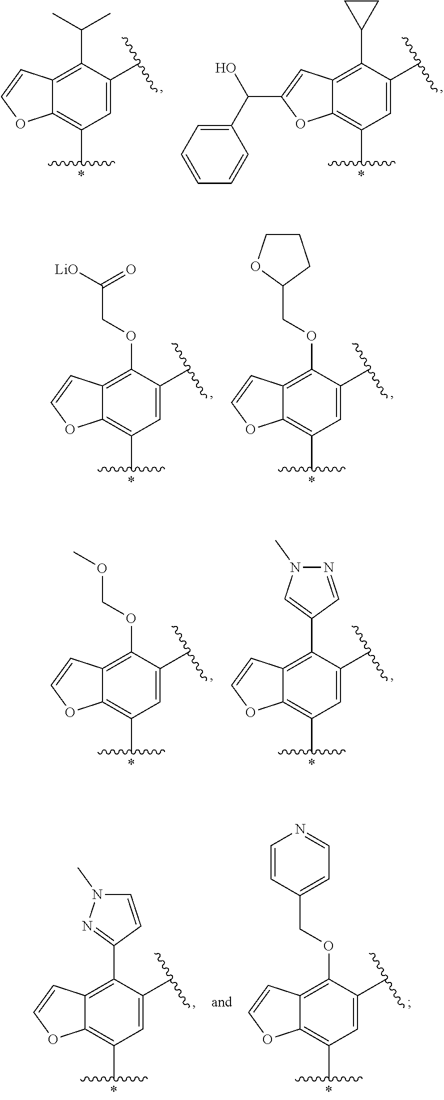

wherein the asterisk (*) indicates the point of attachment to \textcircled{C};

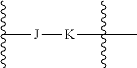

is selected from the group consisting of

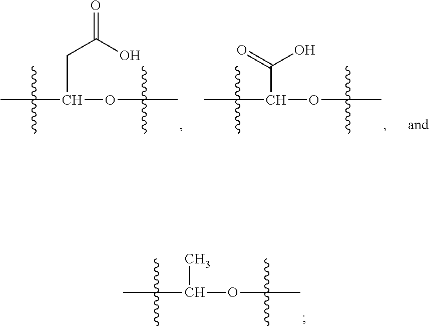

and
\textcircled{C} is selected from the group consisting of

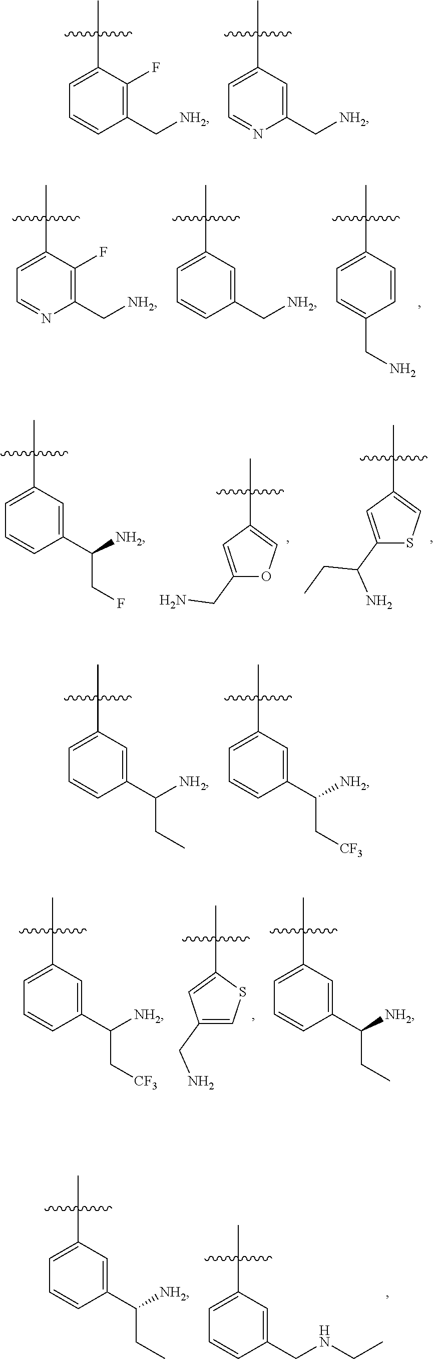

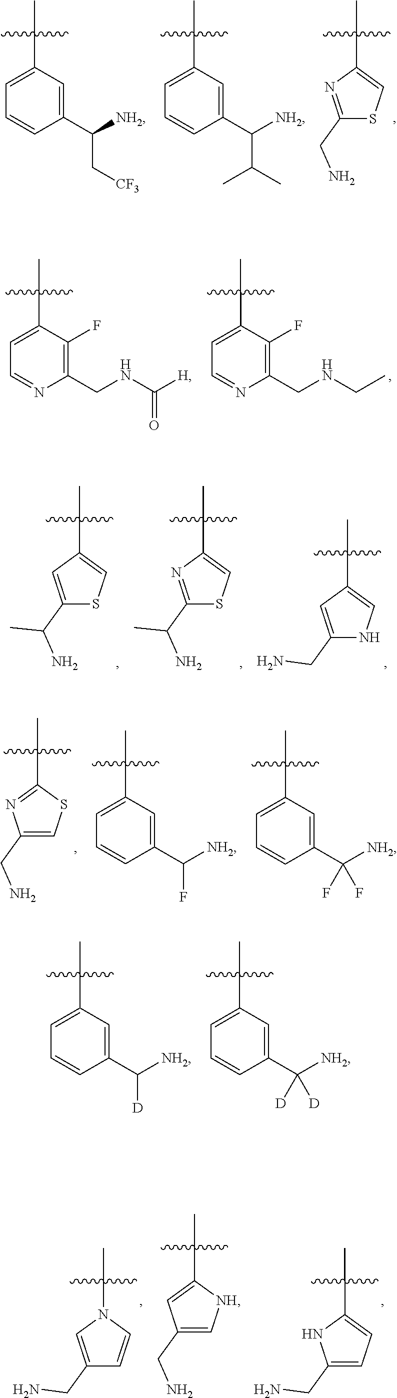

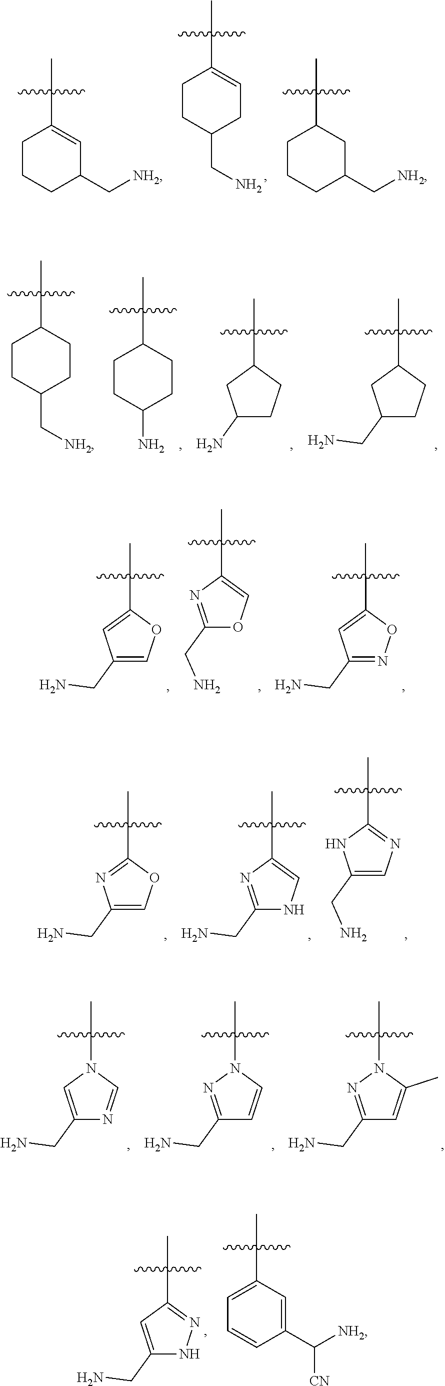

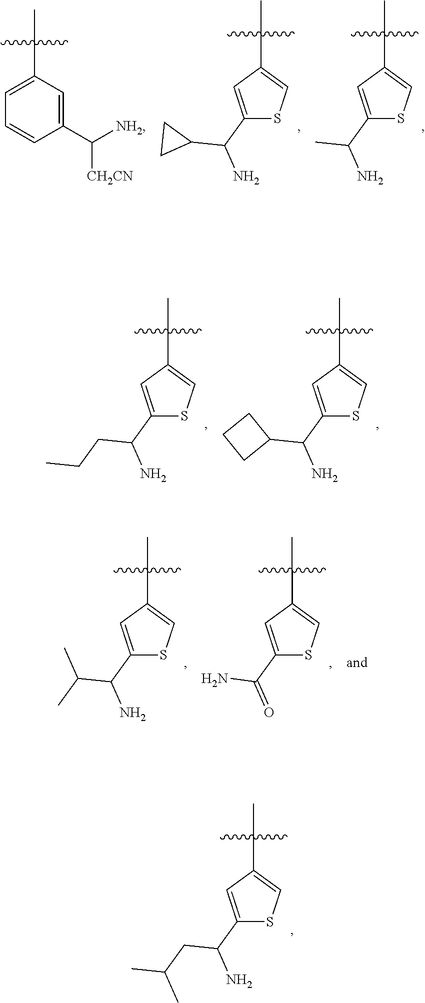

In still further aspects, the invention provides a compound, or a pharmaceutically acceptable salt thereof, selected from the following table:

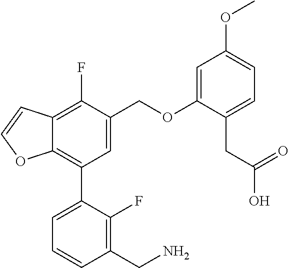

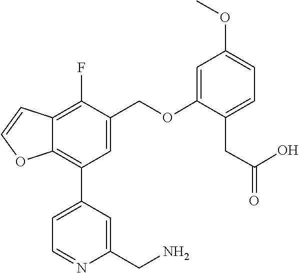

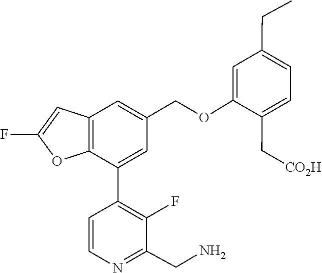

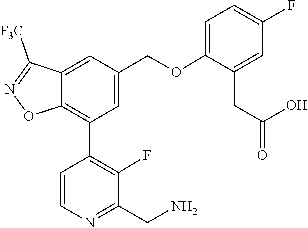

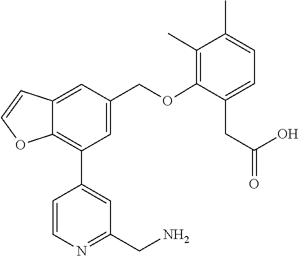

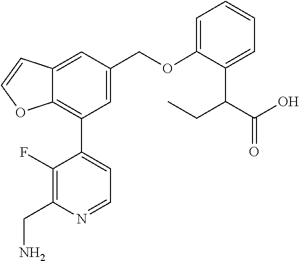

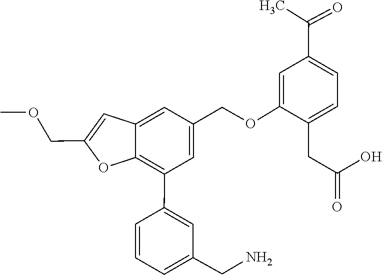

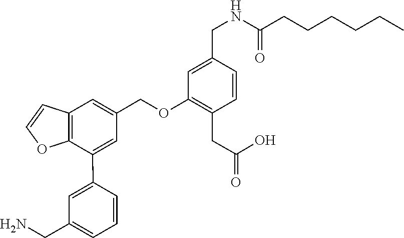

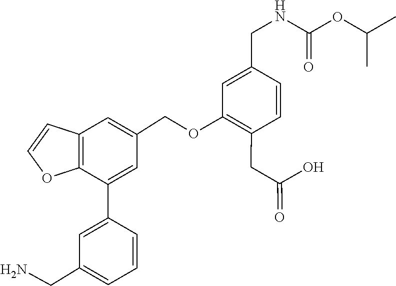

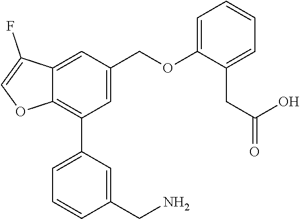

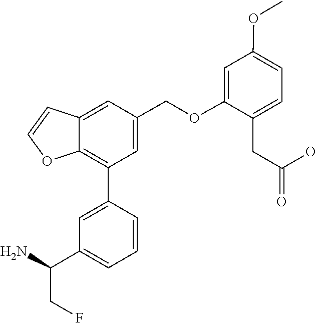

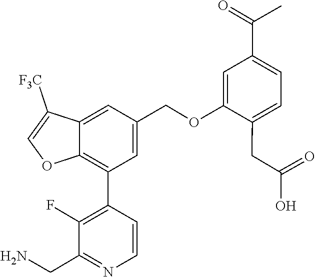

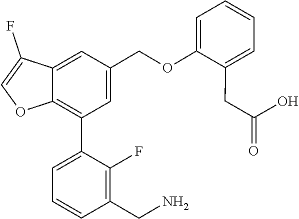

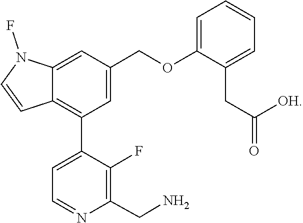

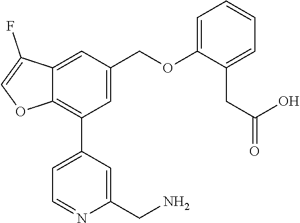

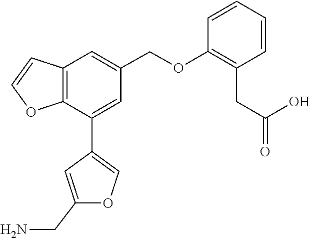

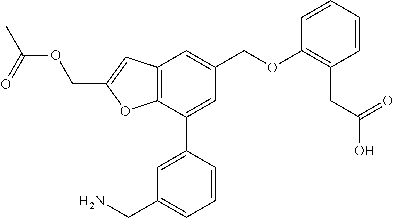

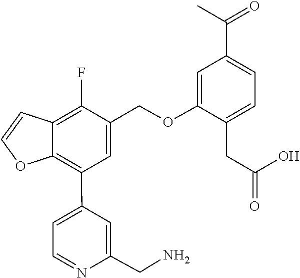

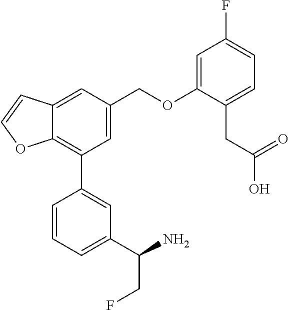

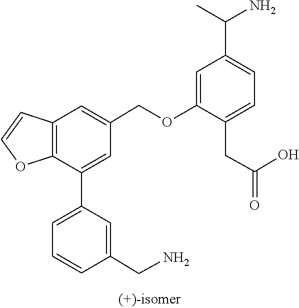

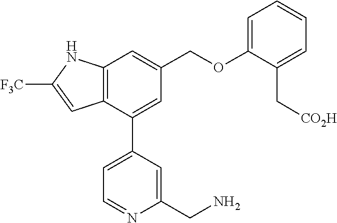

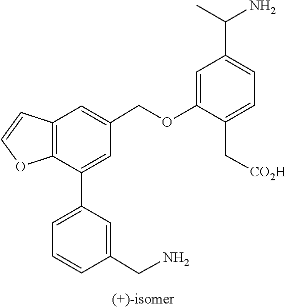

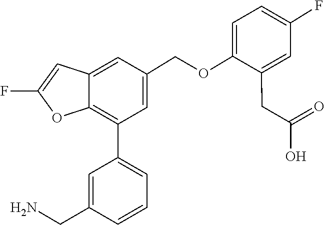

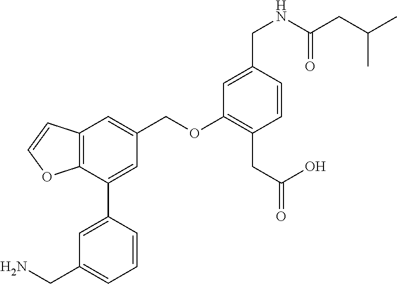

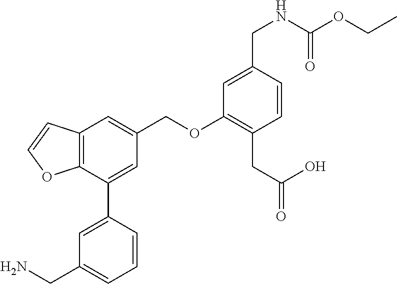

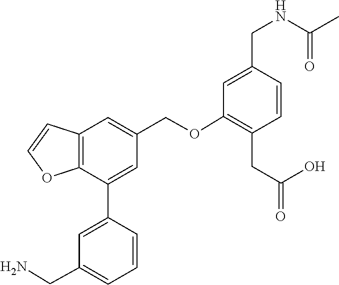

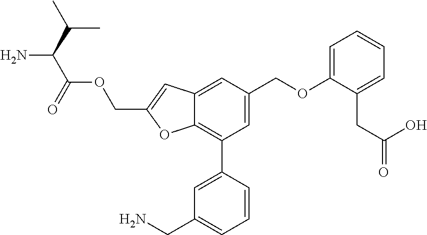

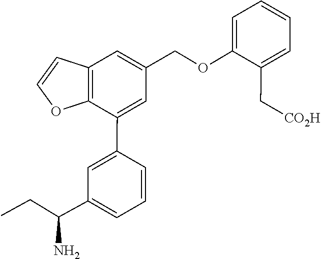

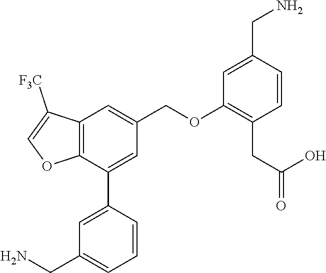

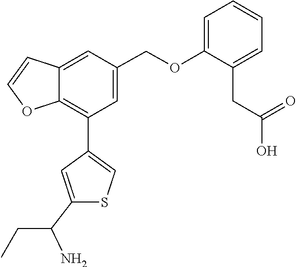

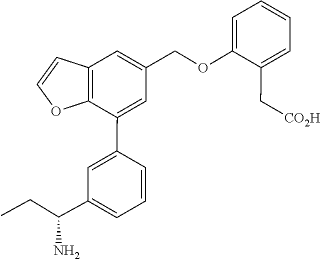

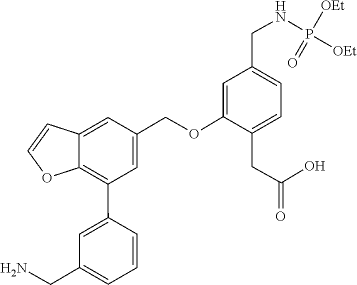

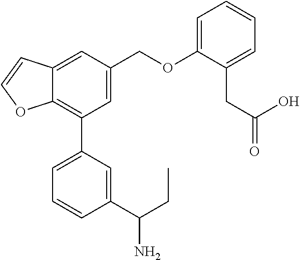

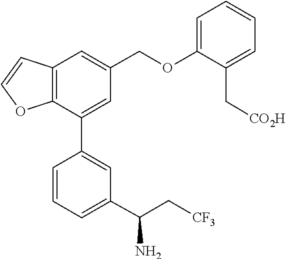

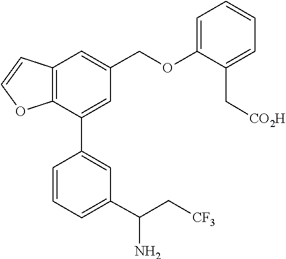

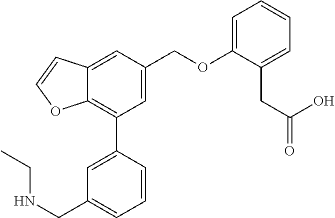

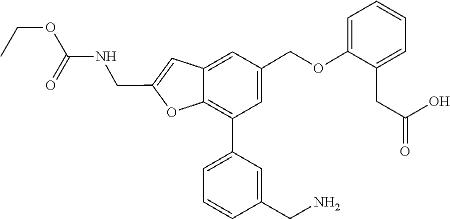

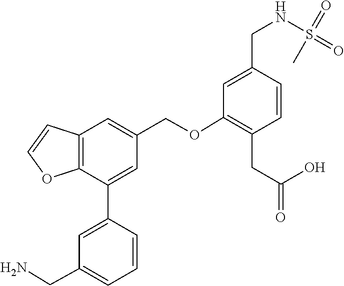

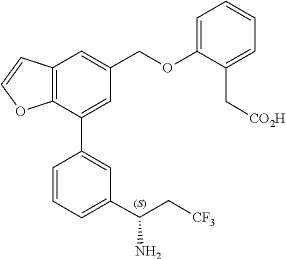

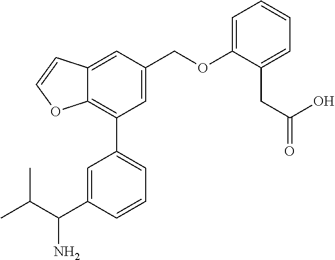

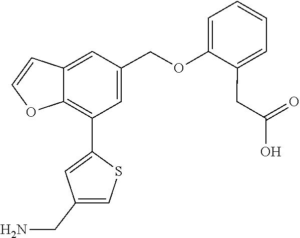

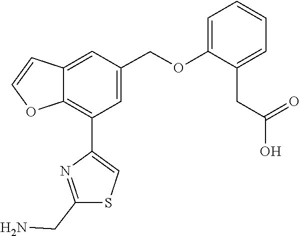

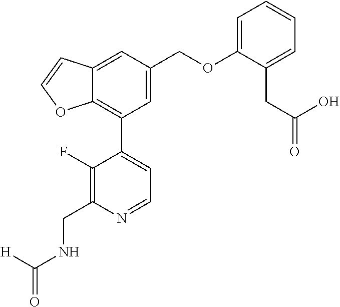

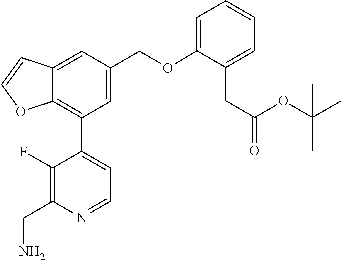

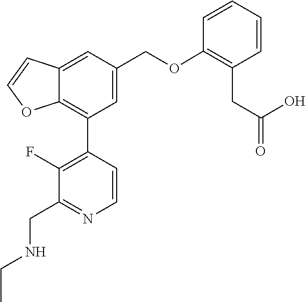

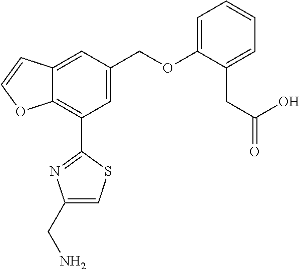

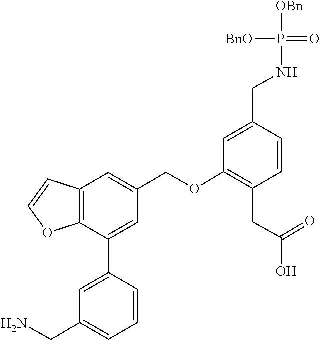

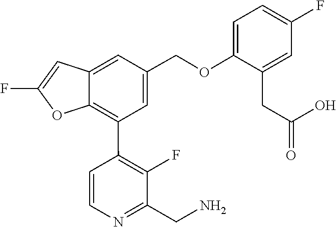

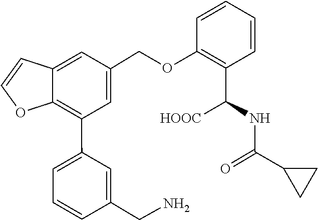

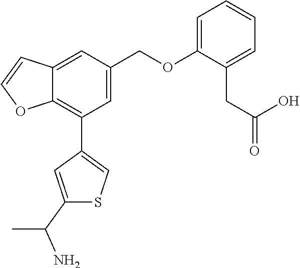

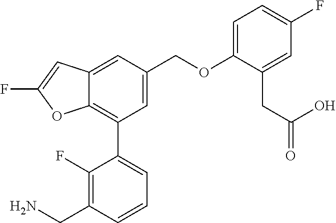

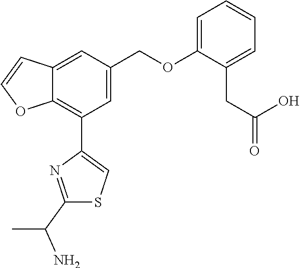

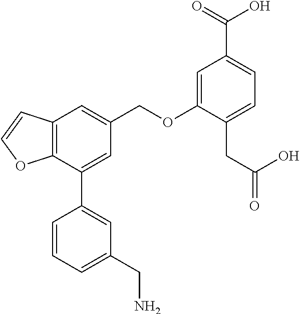

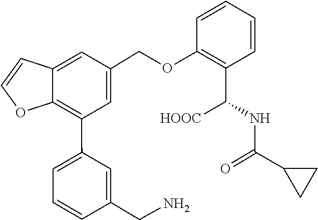

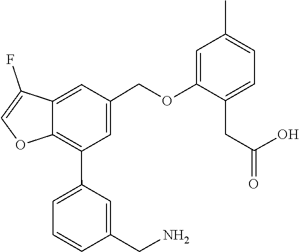

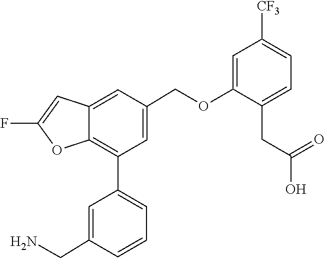

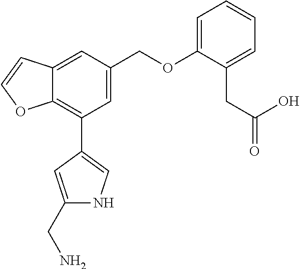

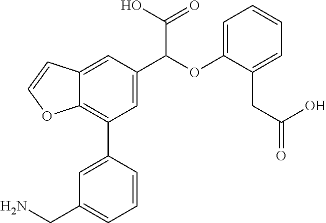

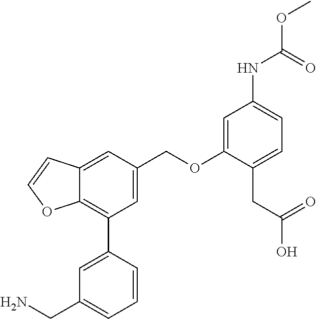

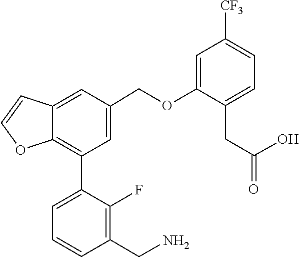

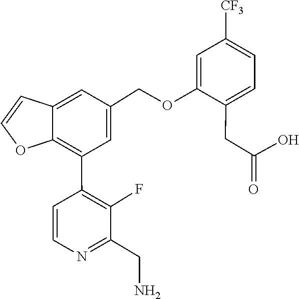

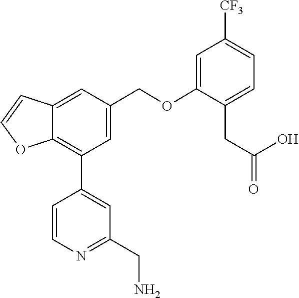

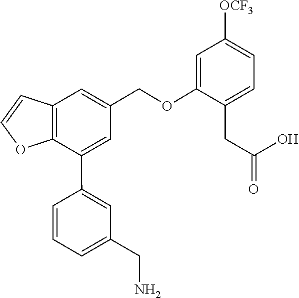

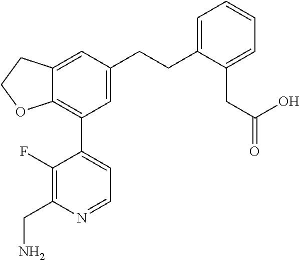

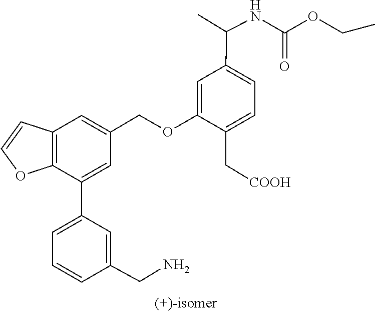

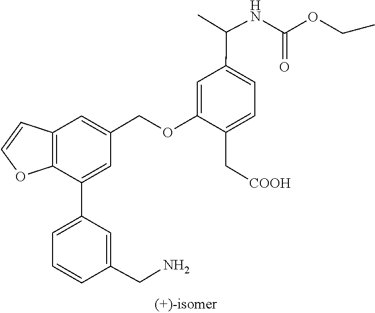

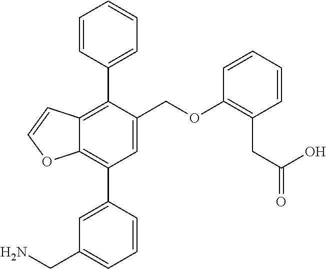

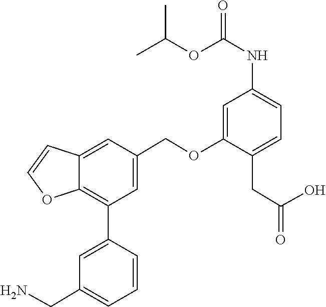

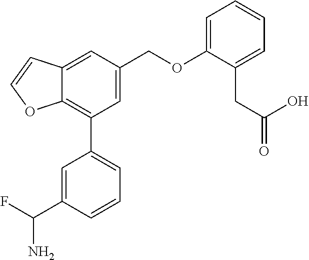

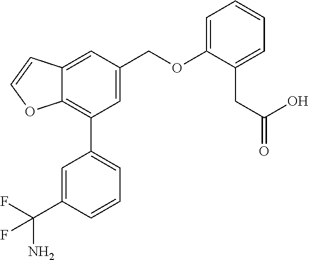

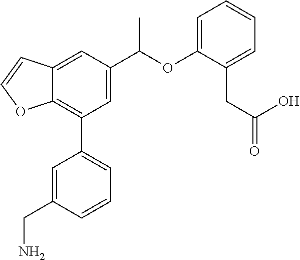

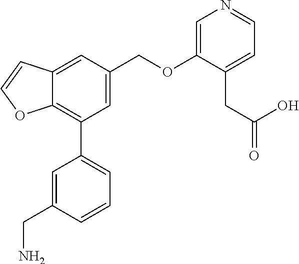

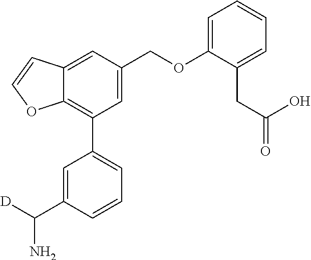

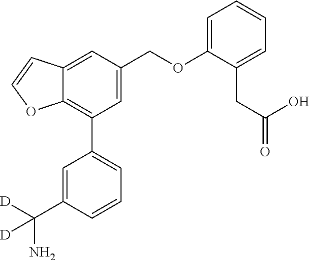

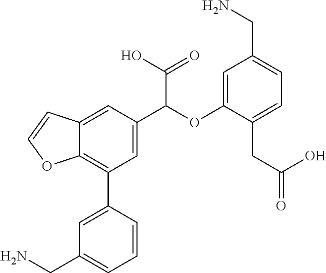

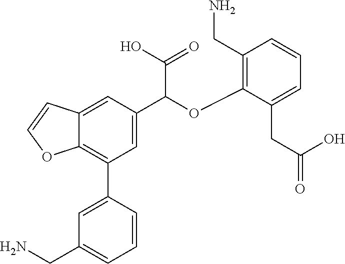

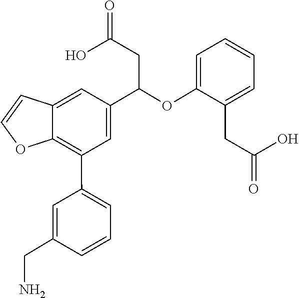

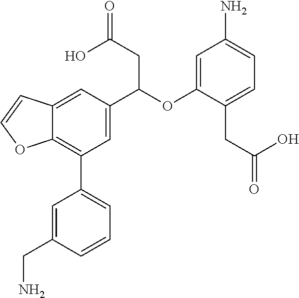

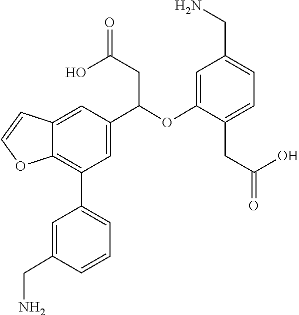

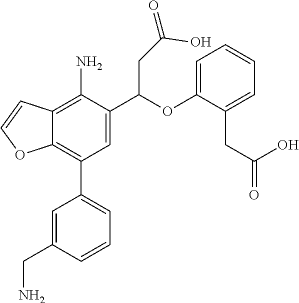

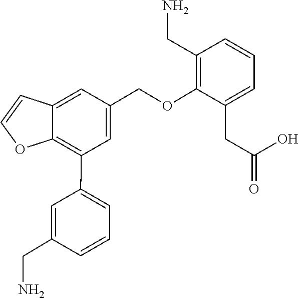

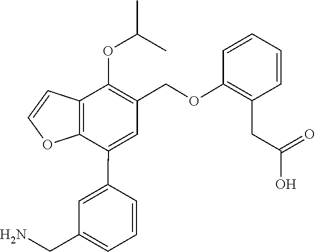

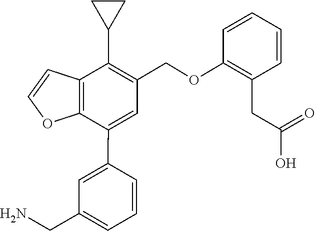

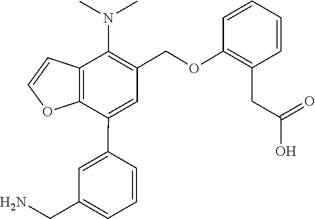

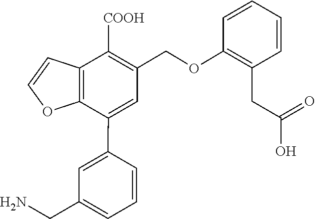

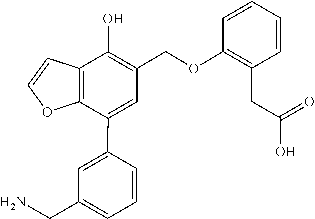

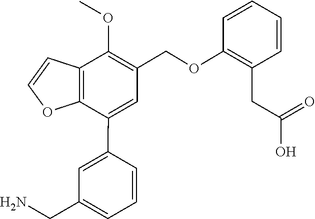

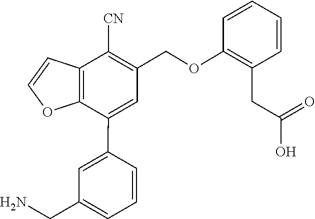

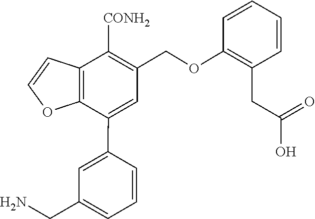

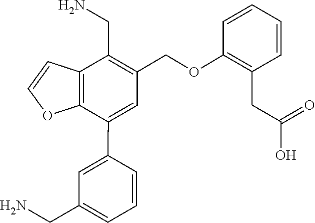

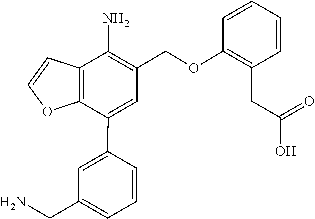

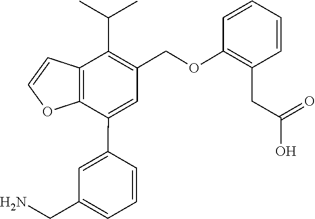

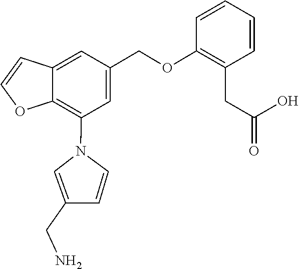

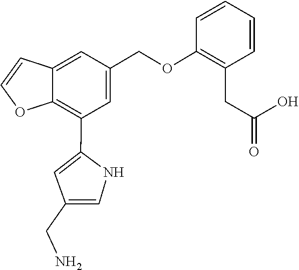

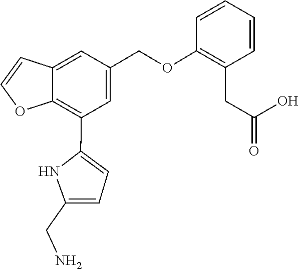

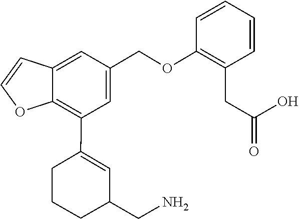

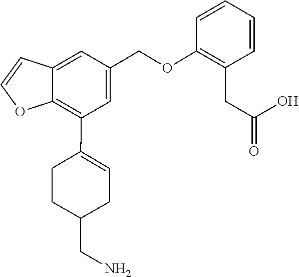

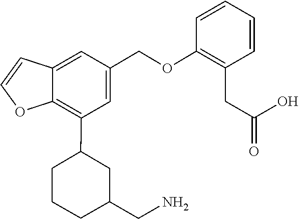

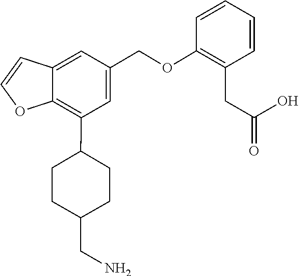

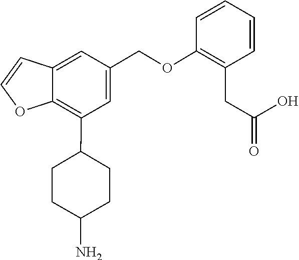

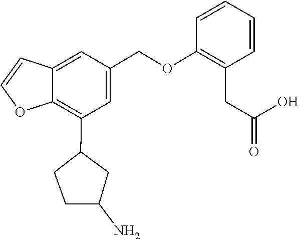

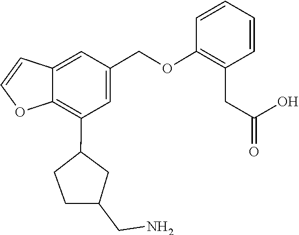

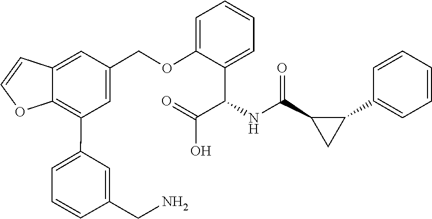

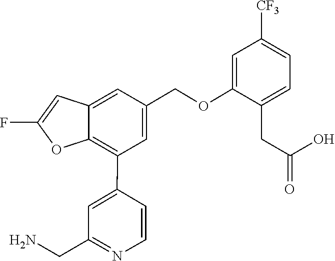

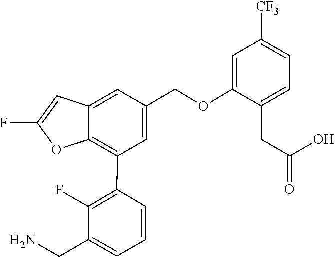

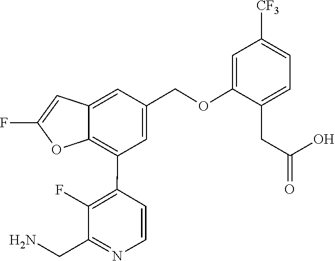

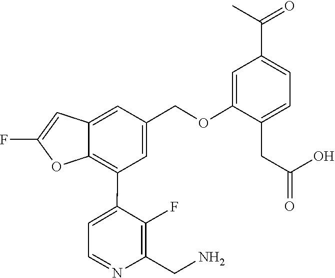

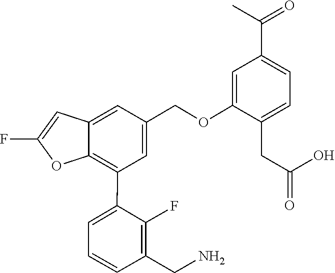

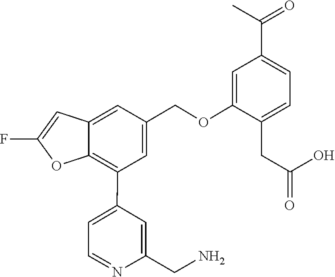

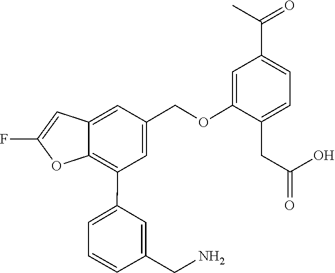

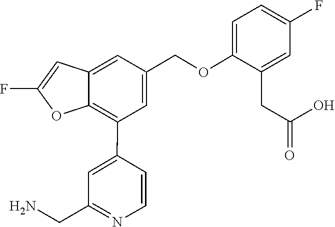

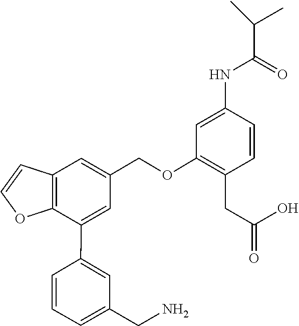

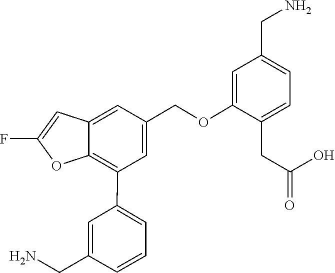

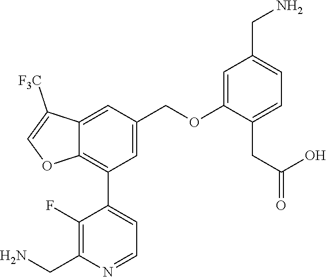

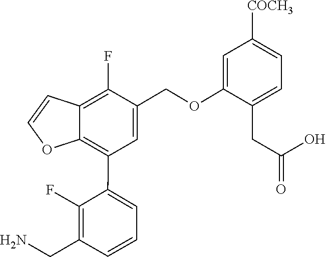

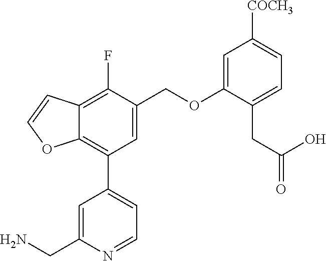

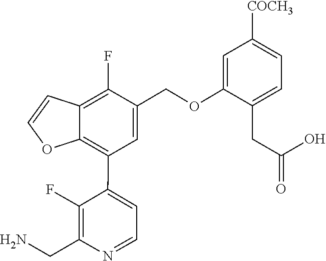

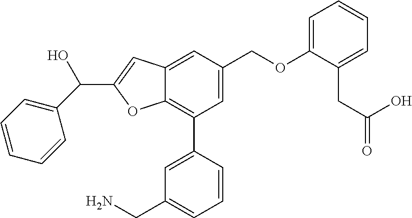

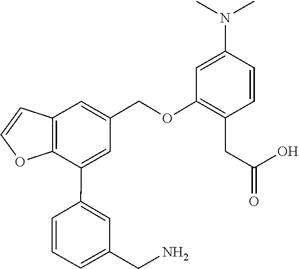

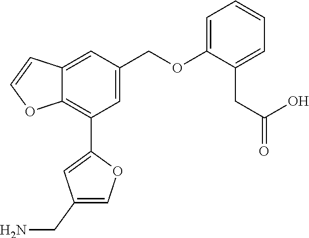

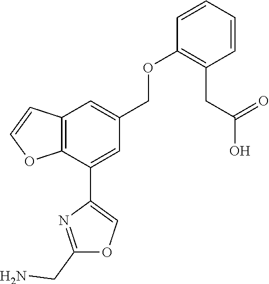

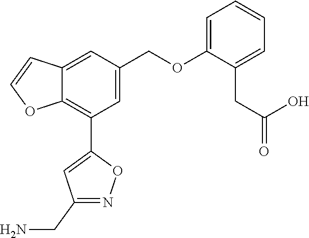

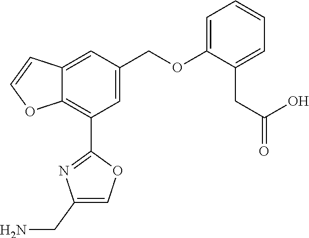

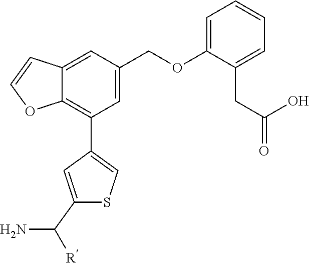

wherein R′ is methyl, propyl,
isopropyl, isobutyl,
cyclopropyl, or cyclobutyl

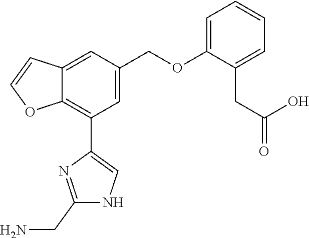 [SMILES: NCc1nc(-c2cc(COc3ccccc3CC(=O)O)cc3ccoc23)c[nH]1]

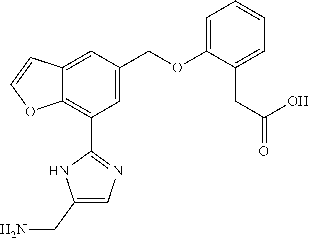 [SMILES: NCc1cnc(-c2cc(COc3ccccc3CC(=O)O)cc3ccoc23)[nH]1]

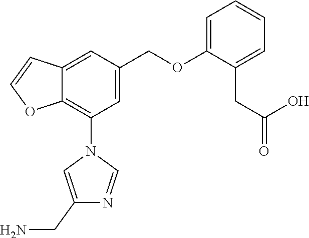 [SMILES: NCc1cn(-c2cc(COc3ccccc3CC(=O)O)cc3ccoc23)cn1]

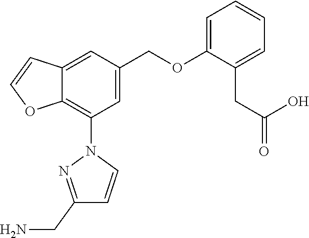 [SMILES: NCc1ccn(-c2cc(COc3ccccc3CC(=O)O)cc3ccoc23)n1]

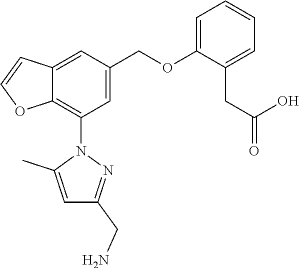 [SMILES: Cc1cc(CN)nn1-c1cc(COc2ccccc2CC(=O)O)cc2ccoc12]

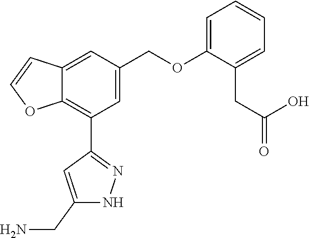 [SMILES: NCc1cc(-c2cc(COc3ccccc3CC(=O)O)cc3ccoc23)n[nH]1]

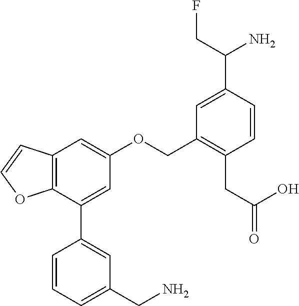 [SMILES: NCc1cccc(-c2cc(OCc3cc(C(N)CF)ccc3CC(=O)O)cc3ccoc23)c1]

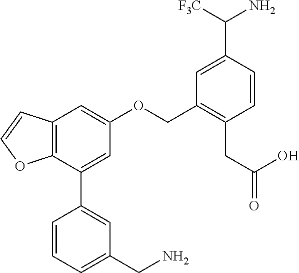 [SMILES: NCc1cccc(-c2cc(OCc3cc(C(N)C(F)(F)F)ccc3CC(=O)O)cc3ccoc23)c1]

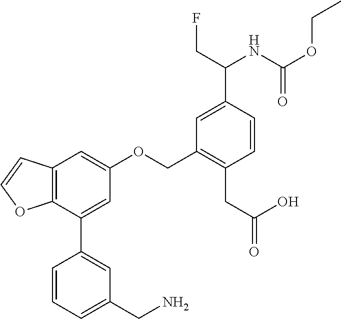 [SMILES: CCOC(=O)NC(CF)c1ccc(CC(=O)O)c(COc2cc(-c3cccc(CN)c3)c3occc3c2)c1]

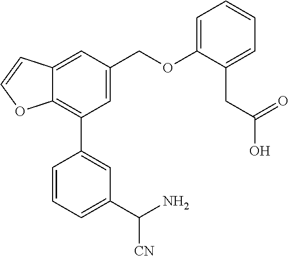 [SMILES: N#CC(N)c1cccc(-c2cc(COc3ccccc3CC(=O)O)cc3ccoc23)c1]

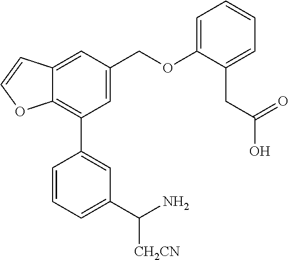

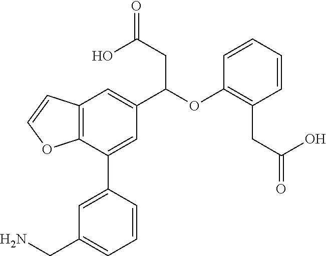 [SMILES: NCc1cccc(-c2cc(C(CC(=O)O)Oc3ccccc3CC(=O)O)cc3ccoc23)c1]

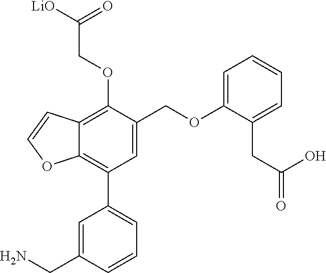

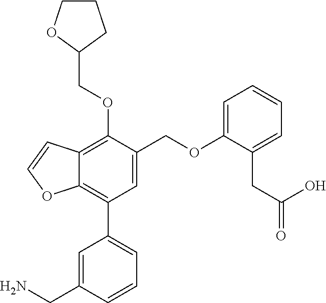 [SMILES: NCc1cccc(-c2cc(COc3ccccc3CC(=O)O)c(OCC3CCCO3)c3ccoc23)c1]

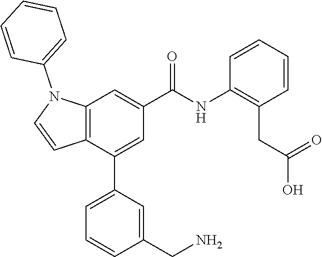 [SMILES: NCc1cccc(-c2cc(C(=O)Nc3ccccc3CC(=O)O)cc3c2ccn3-c2ccccc2)c1]

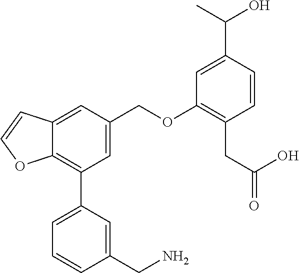 [SMILES: CC(O)c1ccc(CC(=O)O)c(OCc2cc(-c3cccc(CN)c3)c3occc3c2)c1]

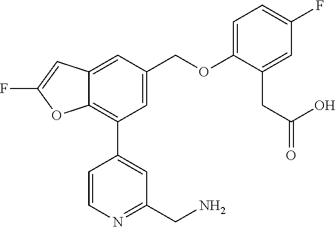 [SMILES: NCc1cc(-c2cc(COc3ccc(F)cc3CC(=O)O)cc3cc(F)oc23)ccn1]

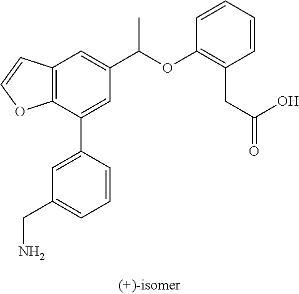 [SMILES: CC(Oc1ccccc1CC(=O)O)c1cc(-c2cccc(CN)c2)c2occc2c1]

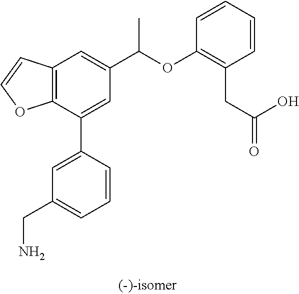 [SMILES: CC(Oc1ccccc1CC(=O)O)c1cc(-c2cccc(CN)c2)c2occc2c1]

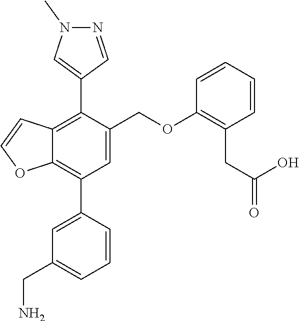 [SMILES: Cn1cc(-c2c(COc3ccccc3CC(=O)O)cc(-c3cccc(CN)c3)c3occc23)cn1]

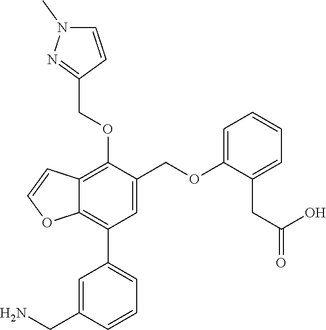 [SMILES: Cn1ccc(COc2c(COc3ccccc3CC(=O)O)cc(-c3cccc(CN)c3)c3occc23)n1]

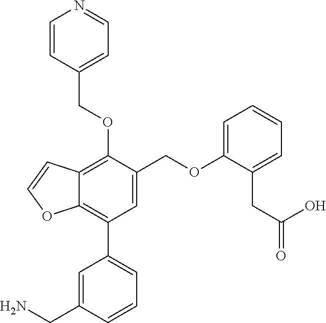 [SMILES: NCc1cccc(-c2cc(COc3ccccc3CC(=O)O)c(OCc3ccncc3)c3ccoc23)c1]

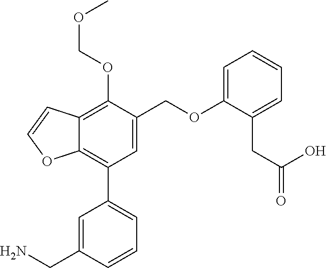 [SMILES: COCOc1c(COc2ccccc2CC(=O)O)cc(-c2cccc(CN)c2)c2occc12]

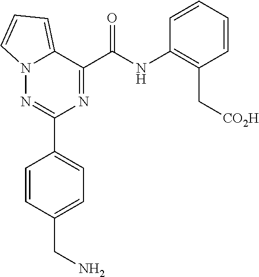 [SMILES: NCc1ccc(-c2nc(C(=O)Nc3ccccc3CC(=O)O)c3cccn3n2)cc1]

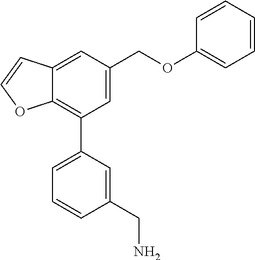 [SMILES: NCc1cccc(-c2cc(COc3ccccc3)cc3ccoc23)c1]

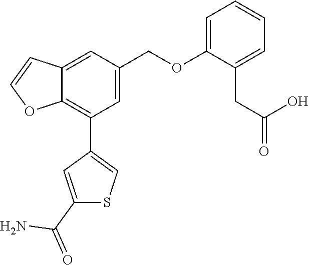 [SMILES: NC(=O)c1cc(-c2cc(COc3ccccc3CC(=O)O)cc3ccoc23)cs1]

In certain aspects, the invention provides a pharmaceutical composition, comprising a compound of the invention, or a pharmaceutically acceptable salt thereof, and a pharmaceutically acceptable carrier.

In certain aspects, the invention provides methods of treating a disease or condition characterized by aberrant complement system activity, comprising administering to a subject in need thereof a therapeutically effective amount of a compound the invention, or a pharmaceutically acceptable salt thereof. In certain embodiments, the disease or condition characterized by aberrant complement system activity is an immunological disorder. In certain embodiments, the disease or condition characterized by aberrant complement system activity is a disease of the central nervous system. In certain embodiments, the disease or condition characterized by aberrant complement system activity is a neurodegenerative disease or neurological disease. In certain embodiments, the disease or condition characterized by aberrant complement system activity is a renal disease. In certain embodiments, the disease or condition characterized by aberrant complement system activity is a cardiovascular disease. In certain embodiments, the disease or condition characterized by aberrant complement system activity is selected from the group consisting of paroxysmal nocturnal hemoglobinuria, atypical hemolytic uremic syndrome, organ transplant rejection, myasthenia gravis, neuromyelitis optica, membranoproliferative glomerulonephritis, dense-deposit disease, cold agglutinin disease, and catastrophic antiphospholipid syndrome. In certain other aspects, the disease or condition characterized by aberrant complement system activity is selected from the group consisting of antineutrophil cytoplasmic antibody (ANCA)-associated vasculitis (AAV), warm autoimmune hemolytic anemia, IgA nephropathy, C3 glomerulonephritis, and focal segmental glomerulosclerosis. In further aspects, the disease or condition characterized by aberrant complement system activity is a hematological disorder. In further aspects, the disease or condition characterized by aberrant complement system activity is an ocular disorder or an eye disorder. In still further aspects, the disease or condition characterized by aberrant complement system activity is macular degeneration, age-related macular degeneration (AMD), macular edema, diabetic macular edema, choroidal neovascularization (CNV), uveitis, Behcet's uveitis, proliferative diabetic retinopathy, non-proliferative diabetic retinopathy, glaucoma, hypertensive retinopathy, a corneal neovascularization disease, post-corneal transplant rejection, a corneal dystrophic disease, an autoimmune dry eye disease, Stevens-Johnson syndrome, Sjogren's syndrome, an environmental dry eye disease, Fuchs' endothelial dystrophy, retinal vein occlusion, or post-operative inflammation.

### DETAILED DESCRIPTION

Inhibitors of the complement system are useful in therapeutic methods and compositions suitable for use in treating disorders of the immune, renal, cardiovascular, and neurological systems. Provided herein are compounds of formulae (I)-(IV) and pharmaceutically acceptable salts thereof that are useful in treating or preventing a disease or condition characterized by aberrant activity of the complement system.

### Definitions

The articles “a” and “an” are used herein to refer to one or to more than one (i.e., to at least one) of the grammatical object of the article. By way of example, “an element” means one element or more than one element.

Certain compounds contained in compositions of the present invention may exist in particular geometric or stereoisomeric forms. In addition, compounds of the present invention may also be optically active. The present invention contemplates all such compounds, including cis- and trans-isomers, (R)- and (S)-enantiomers, diastereoisomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The phrase “protecting group”, as used herein, means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

For purposes of the invention, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I. As another example, the salt may comprise less than one inorganic or organic acid molecule per molecule of base, such as two molecules of compound of Formula I per molecule of tartaric acid.

The term “prodrug” as used herein refers to a compound that can be metabolized in vivo to provide a compound of the invention. Thus prodrugs include compounds that can be prepared by modifying one or more functional groups in a compound of the invention to provide a corresponding compound that can be metabolized in vivo to provide a compound of the invention. Such modifications are known in the art. For example, one or more hydroxyl groups or amine groups in a compound of the invention can be acylated with alkyl-C groups or with residues from amino acids to provide a prodrug.

Prodrug forms of a compound bearing various nitrogen-containing functional groups (amino, hydroxyamino, amide, etc.) may include the following types of derivatives, where each Rₚ group individually may be hydrogen, substituted or unsubstituted alkyl, aryl, alkenyl, alkynyl, heterocycle, alkylaryl, arylalkyl, aralkenyl, aralkynyl, cycloalkyl or cycloalkenyl.
(a) Carboxamides, represented as —NHC(O)Rₚ
(b) Carbamates, represented as —NHC(O)ORₚ
(c) (Acyloxy)alkyl Carbamates, represented as NHC(O)OROC(O)Rₚ
(d) Enamines, represented as —NHCR(═CHCO₂Rₚ) or —NHCR(═CHCONRₚRₚ)
(e) Schiff Bases, represented as —N═CRₚRₚ
(f) Mannich Bases (from carboximide compounds), represented as RCONHCH₂NRₚRₚ.

Preparations of such prodrug derivatives are discussed in various literature sources (examples are: Alexander et al. J. Med. Chem. 1988, 31, 318; Aligas-Martin et al., PCT WO0041531, p. 30).

Prodrug forms of carboxyl-bearing compounds include esters (—CO₂Rₘ), where the Rₘ group corresponds to any alcohol whose release in the body through enzymatic or hydrolytic processes would be at pharmaceutically acceptable levels. Another prodrug derived from a carboxylic acid form of the disclosure may be a quaternary salt type of structure described by Bodor et al., Med. Chem. 1980, 23, 469.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “treat” as used herein means prevent, halt or slow the progression of, or eliminate a disease or condition in a subject. In one embodiment “treat” means halt or slow the progression of, or eliminate a disease or condition in a subject. In one embodiment, “treat” means reduce at least one objective manifestation of a disease or condition in a subject.

The term “effective amount” as used herein refers to an amount that is sufficient to bring about a desired biological effect.

The term “therapeutically effective amount” as used herein refers to an amount that is sufficient to bring about a desired therapeutic effect.

The term “inhibit” as used herein means decrease by an objectively measurable amount or extent. In various embodiments “inhibit” means decrease by at least 5, 10, 20, 30, 40, 50, 60, 70, 80, 90, or 95 percent compared to relevant control. In one embodiment “inhibit” means decrease 100 percent, i.e., halt or eliminate.

The term “subject” as used herein refers to a mammal. In various embodiments, a subject is a mouse, rat, rabbit, cat, dog, pig, sheep, horse, cow, or non-human primate. In one embodiment, a subject is a human.

### Compounds

The present invention provides compounds having the structure of Formula (I), and pharmaceutically acceptable salts thereof:

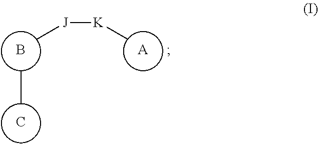

wherein:
A is selected from the group consisting of

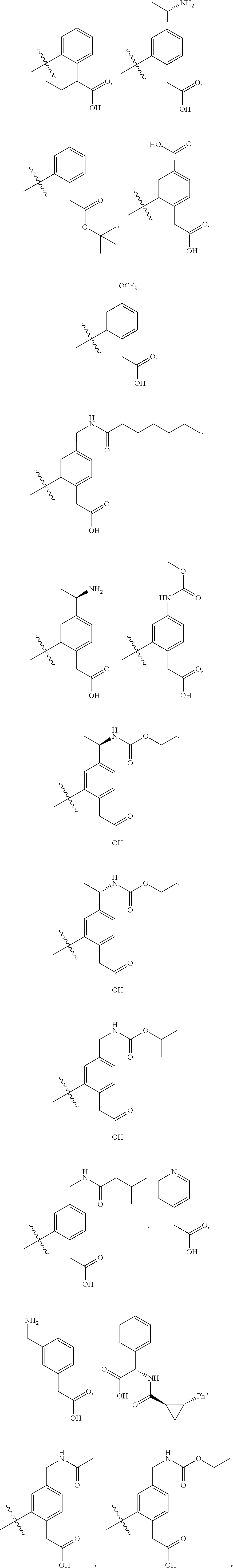

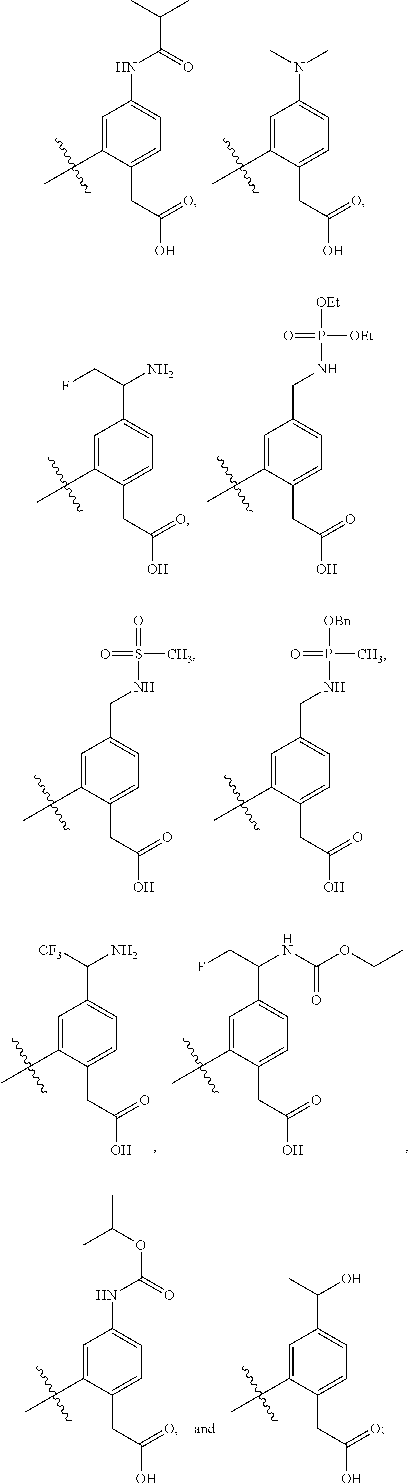

\textcircled{B} is selected from the group consisting of

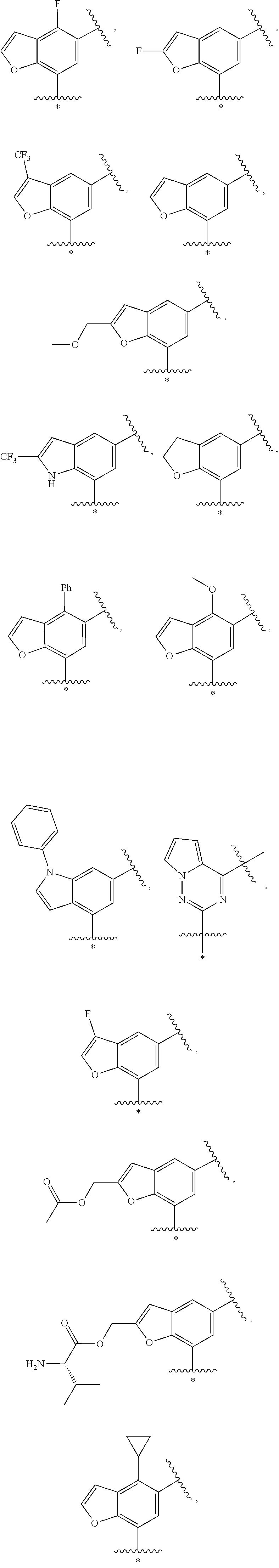

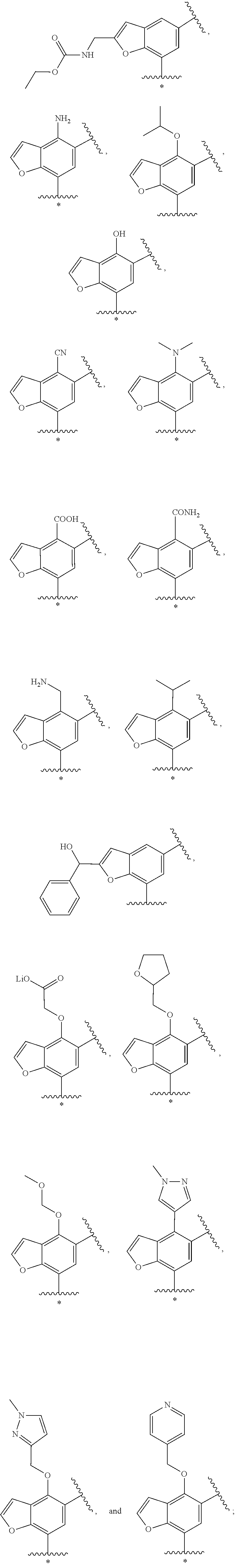

wherein the asterisk (*) indicates the point of attachment to \textcircled{C};

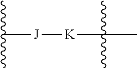

is selected from the group consisting of

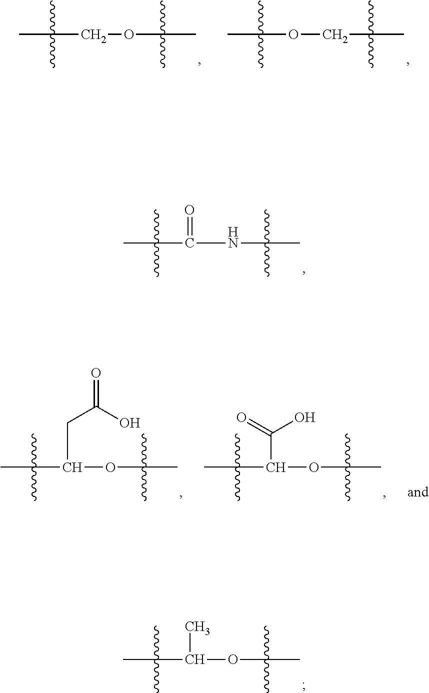

and
\textcircled{C} is selected from the group consisting of

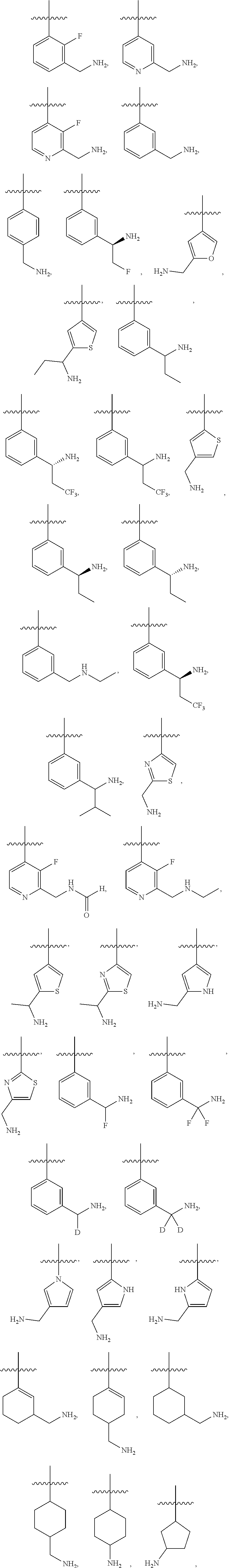

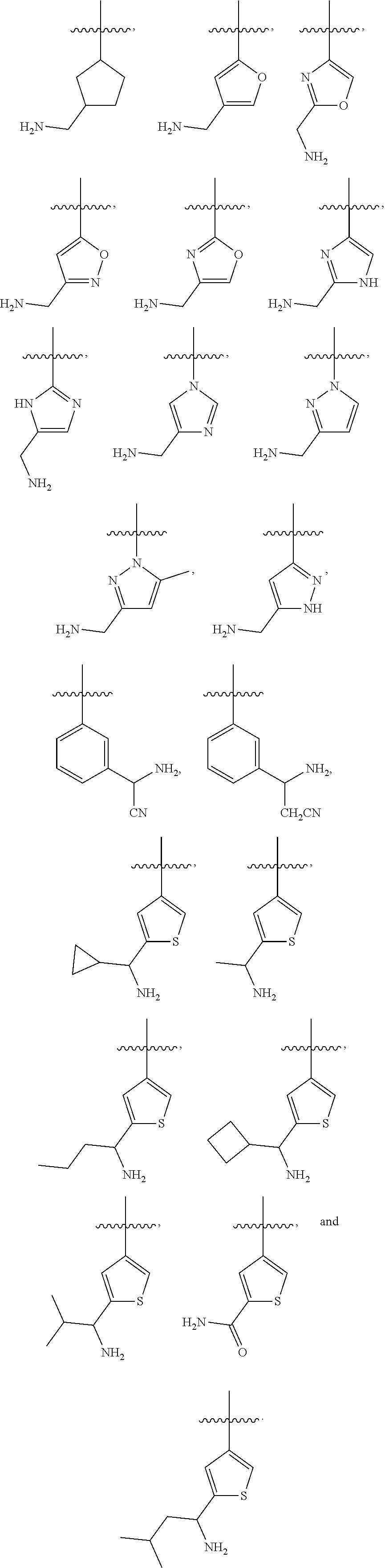

In some embodiments of the compounds of formula (I) \textcircled{A} is selected from the group consisting of

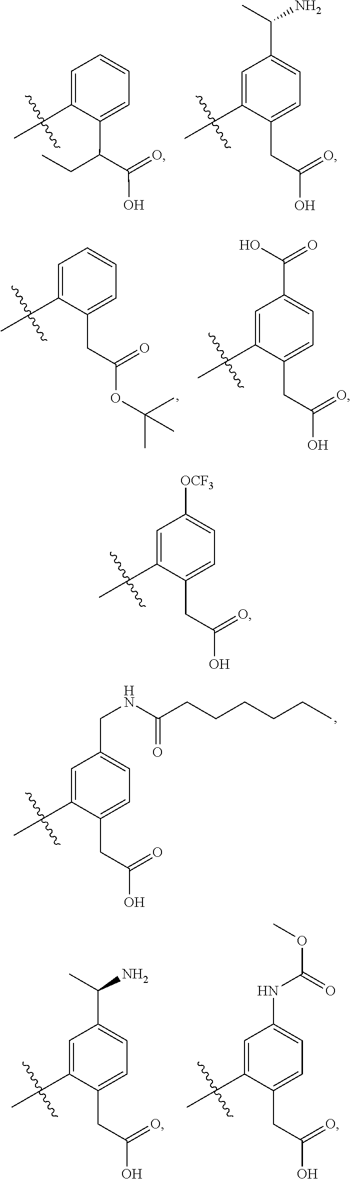

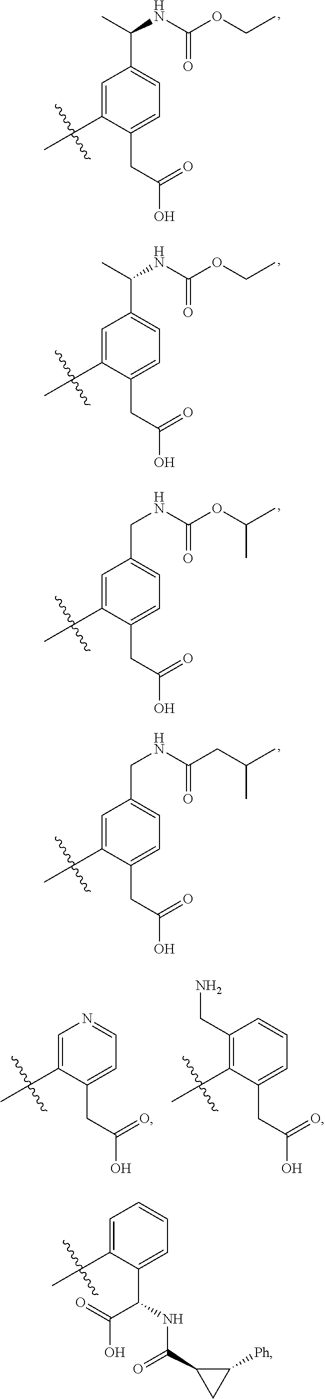

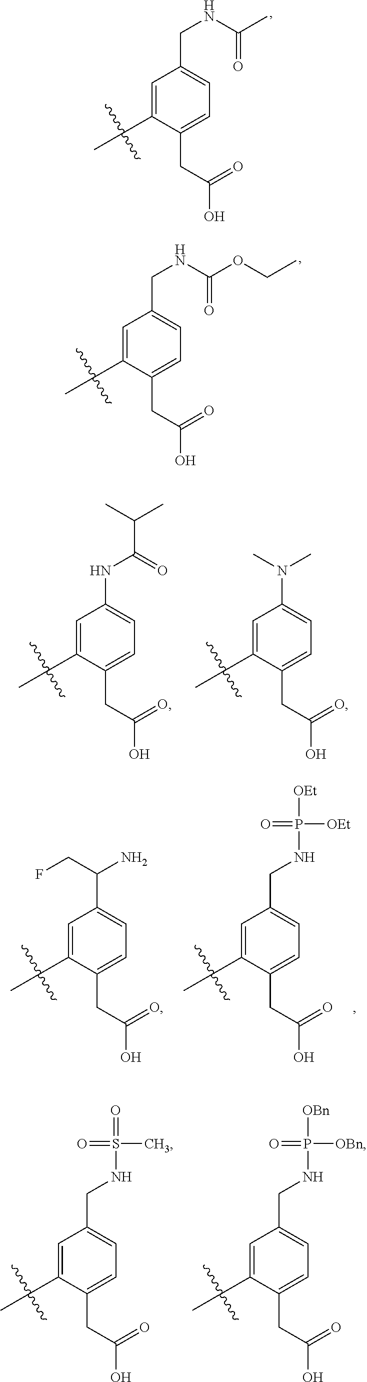

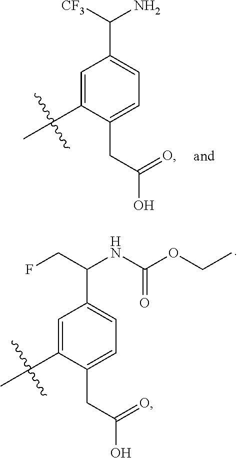 [SMILES: *c1cc(C(N)C(F)(F)F)ccc1CC(=O)O ; *c1cc(C(CF)NC(=O)OCC)ccc1CC(=O)O]

In some embodiments of the compounds of formula (I), \textcircled{B} is

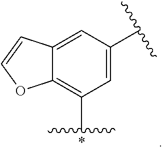 [SMILES: *c1cc(*)c2occc2c1]

In some embodiments of the compounds of formula (I),

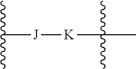

is

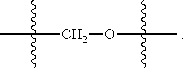 [SMILES: *CO*]

In some embodiments of the compounds of formula (I), \textcircled{C} is selected from the group consisting of

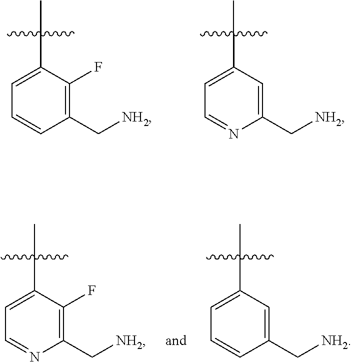 [SMILES: *c1cccc(CN)c1F ; *c1ccnc(CN)c1 ; *c1ccnc(CN)c1F ; *c1cccc(CN)c1]

In preferred embodiments, \textcircled{C} is

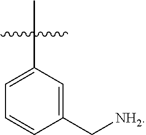 [SMILES: *c1cccc(CN)c1]

The present invention provides compounds having the structure of Formula (II), and pharmaceutically acceptable salts thereof:

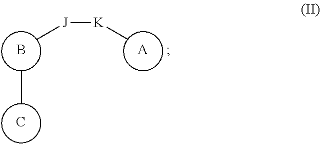

wherein:
\textcircled{A} is selected from the group consisting of

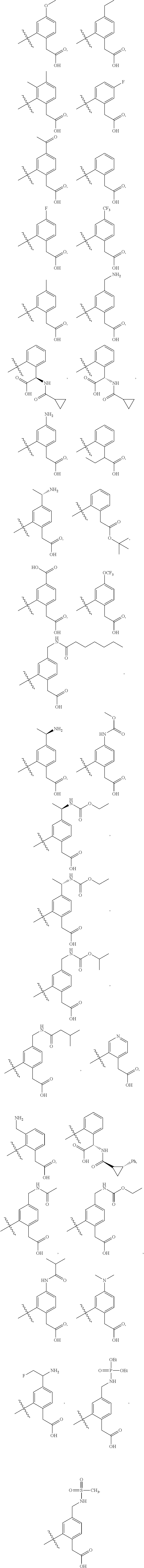

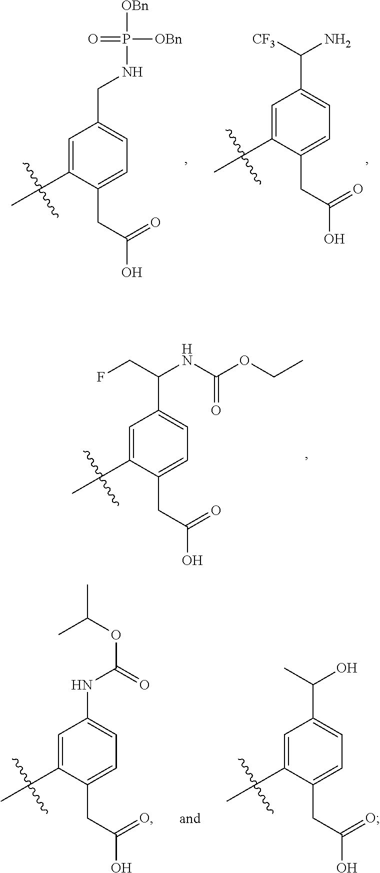 [SMILES: *c1cc(CNP(=O)(OCc2ccccc2)OCc2ccccc2)ccc1CC(=O)O ; *c1cc(C(N)C(F)(F)F)ccc1CC(=O)O ; *c1cc(C(CF)NC(=O)OCC)ccc1CC(=O)O ; *c1cc(NC(=O)OC(C)C)ccc1CC(=O)O ; *c1cc(C(C)O)ccc1CC(=O)O]

\textcircled{B} is selected from the group consisting of

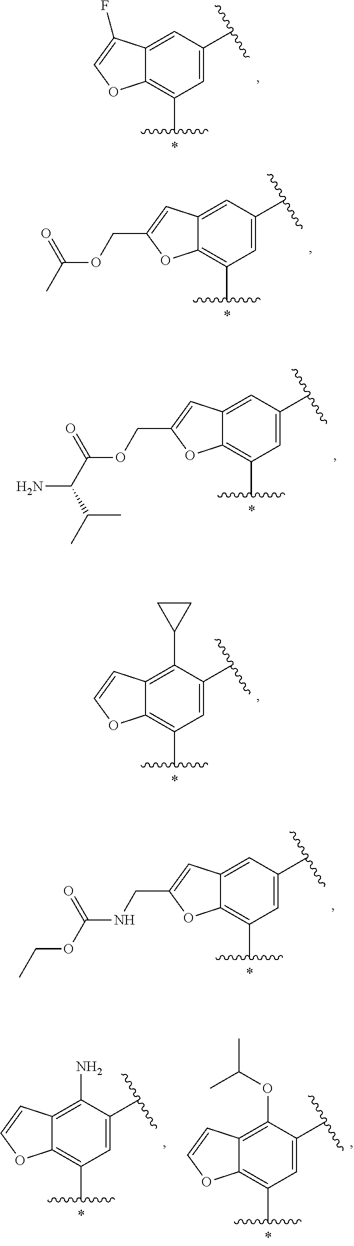

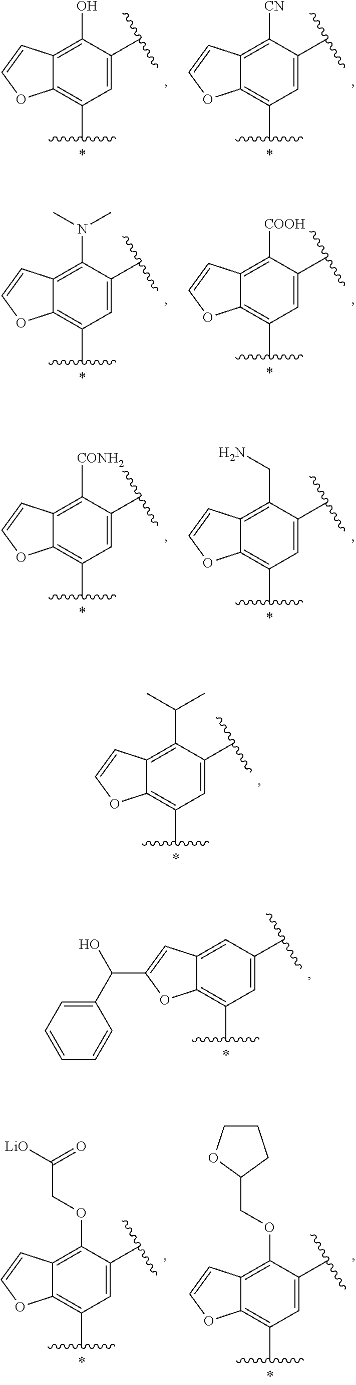

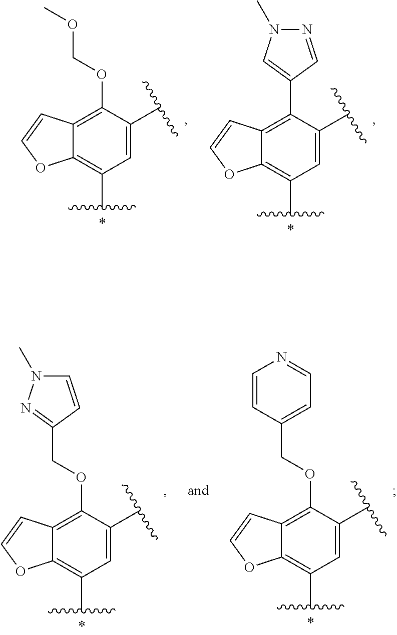 [SMILES: *c1cc(*)c2occc2c1OCOC ; *c1cc(*)c2occc2c1-c1cnn(C)c1 ; *c1cc(*)c2occc2c1OCc1ccn(C)n1 ; *c1cc(*)c2occc2c1OCc1ccncc1]

wherein the asterisk (*) indicates the point of attachment to \textcircled{C};

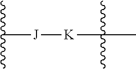

is selected from the group consisting of

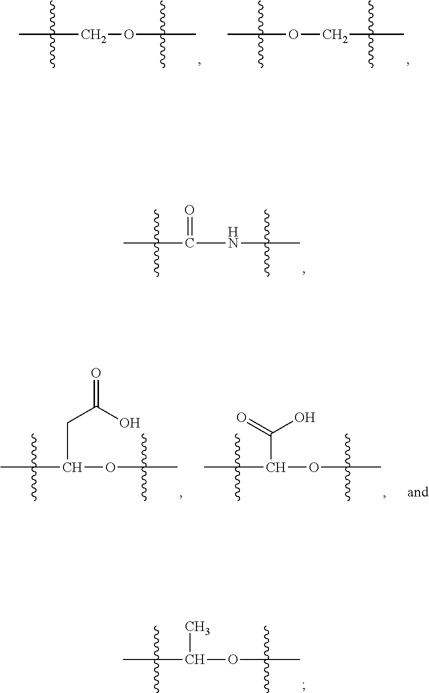

\textcircled{C} is selected from the group consisting of

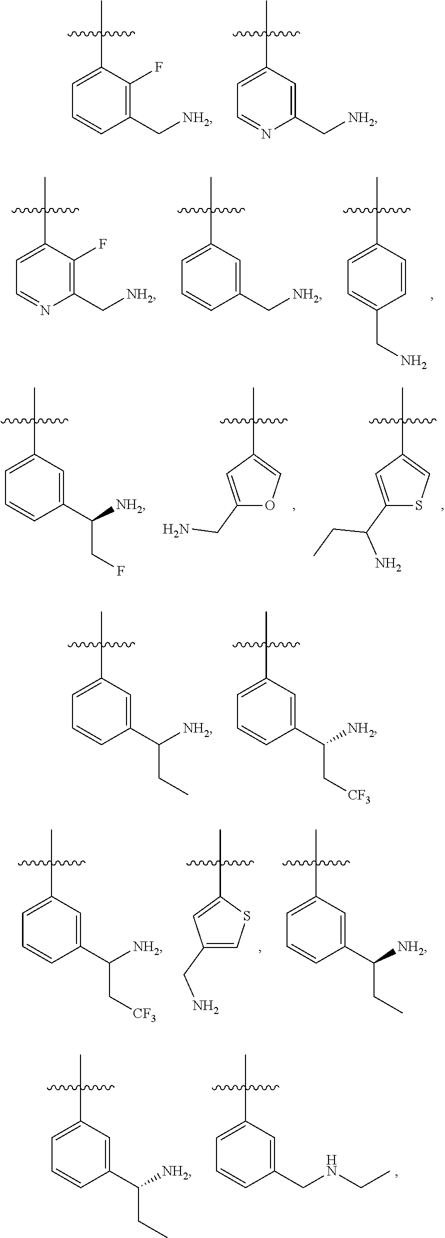

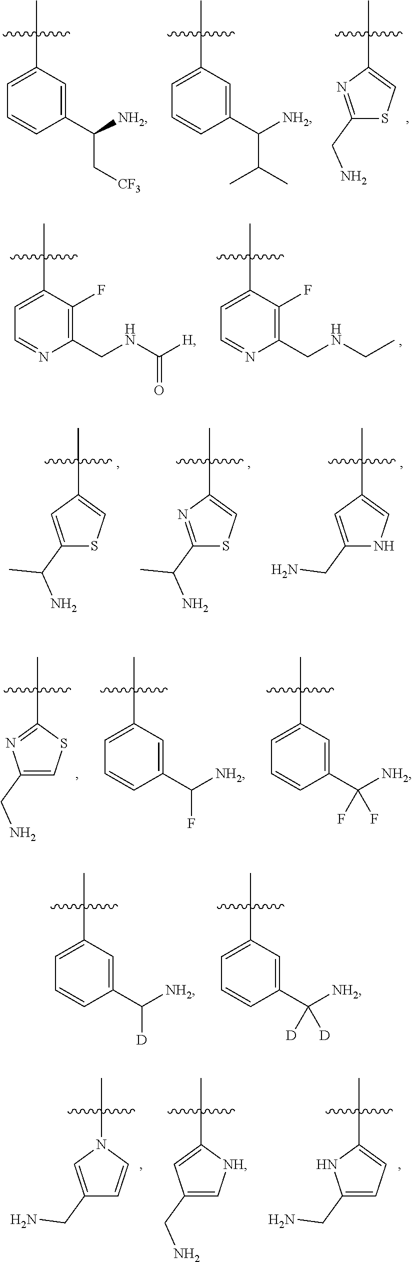

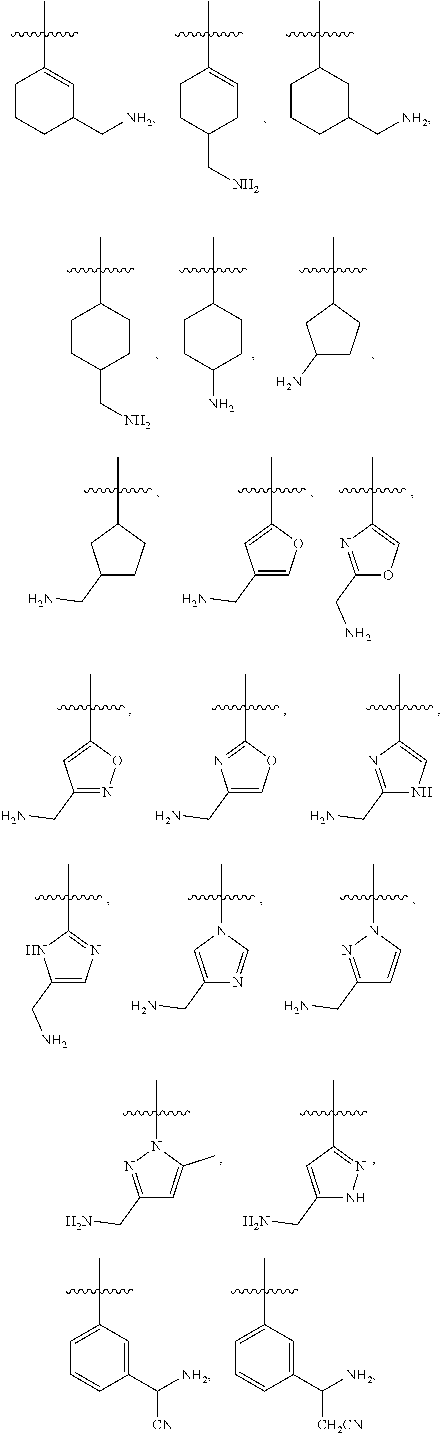

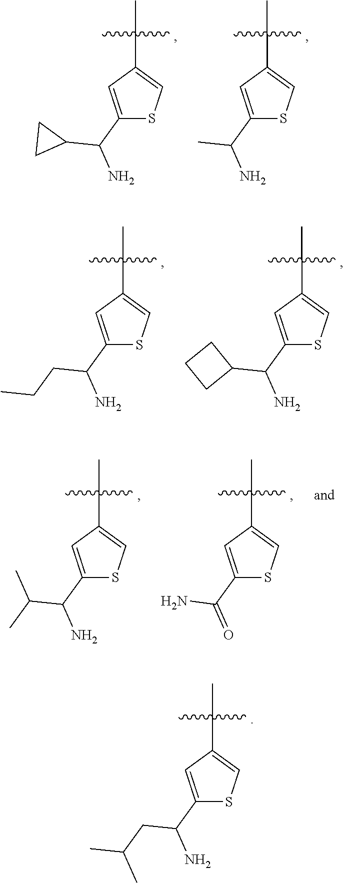

In some embodiments of the compounds of formula (II), \textcircled{B} is selected from the group consisting of

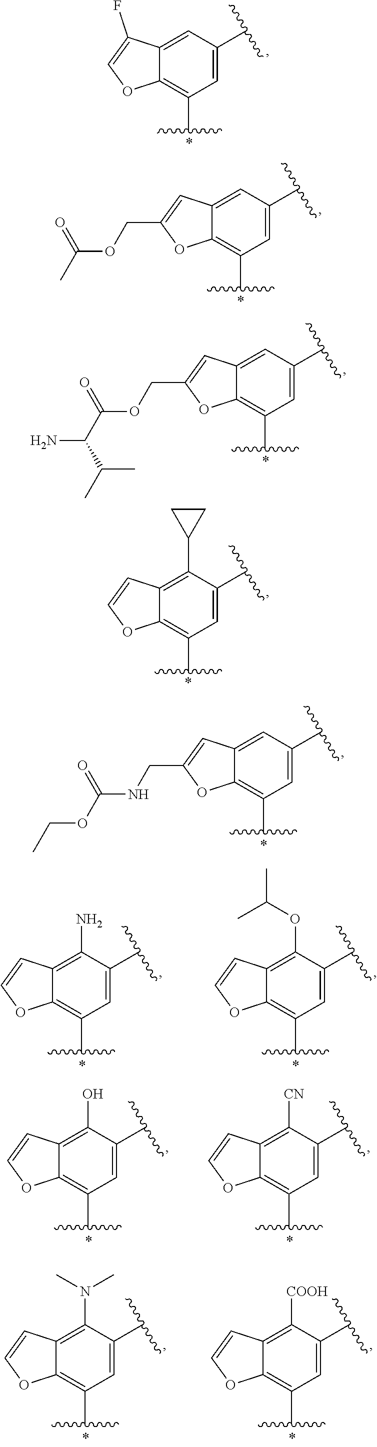

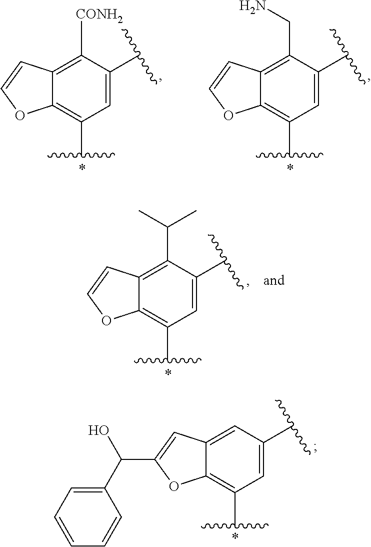 [SMILES: *c1cc(*)c2occc2c1C(N)=O ; *c1cc(*)c2occc2c1CN ; *c1cc(*)c2occc2c1C(C)C ; *c1cc(*)c2oc(C(O)c3ccccc3)cc2c1]

wherein the asterisk (*) indicates the point of attachment to \textcircled{C}.

In some embodiments of the compounds of formula (II), \textcircled{A} is

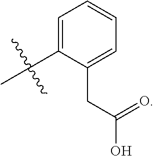 [SMILES: *c1ccccc1CC(=O)O]

In some embodiments of the compounds of formula (II),

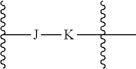

is

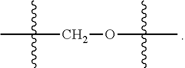 [SMILES: *CO*]

In some embodiments of the compounds of formula (II), \textcircled{C} is selected from the group consisting of

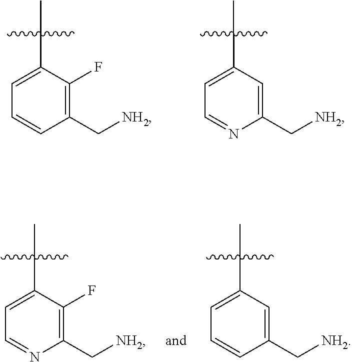 [SMILES: *c1cccc(CN)c1F ; *c1ccnc(CN)c1 ; *c1ccnc(CN)c1F ; *c1cccc(CN)c1]

In preferred embodiments, \textcircled{C} is

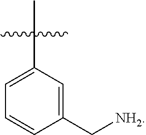 [SMILES: *c1cccc(CN)c1]

The present invention provides compounds having the structure of Formula (III), and pharmaceutically acceptable salts thereof:

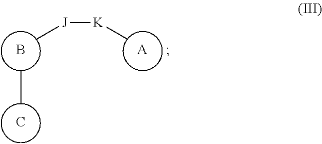

wherein:
\textcircled{A} is selected from the group consisting of

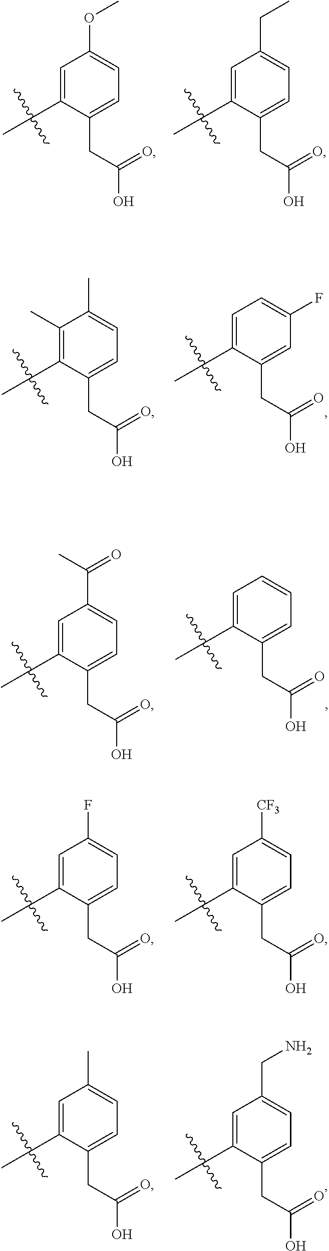

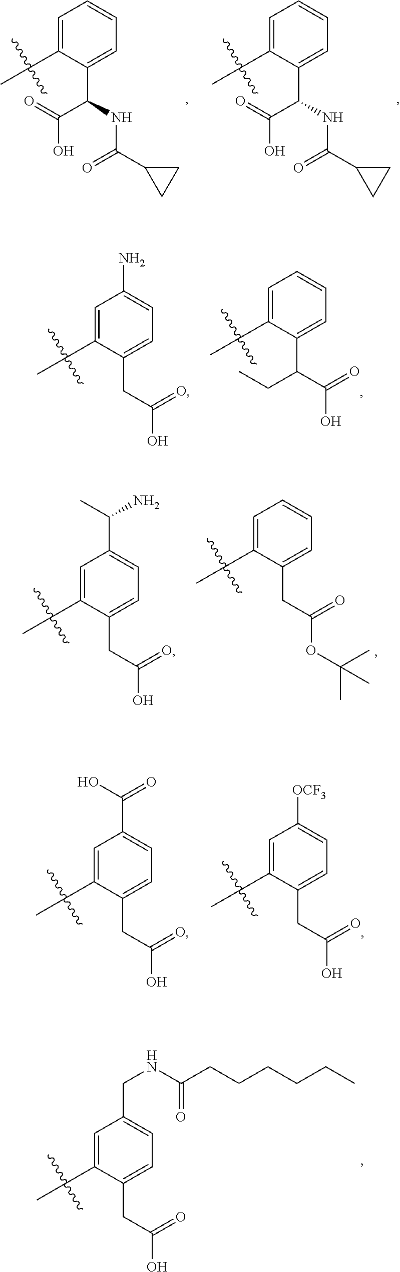

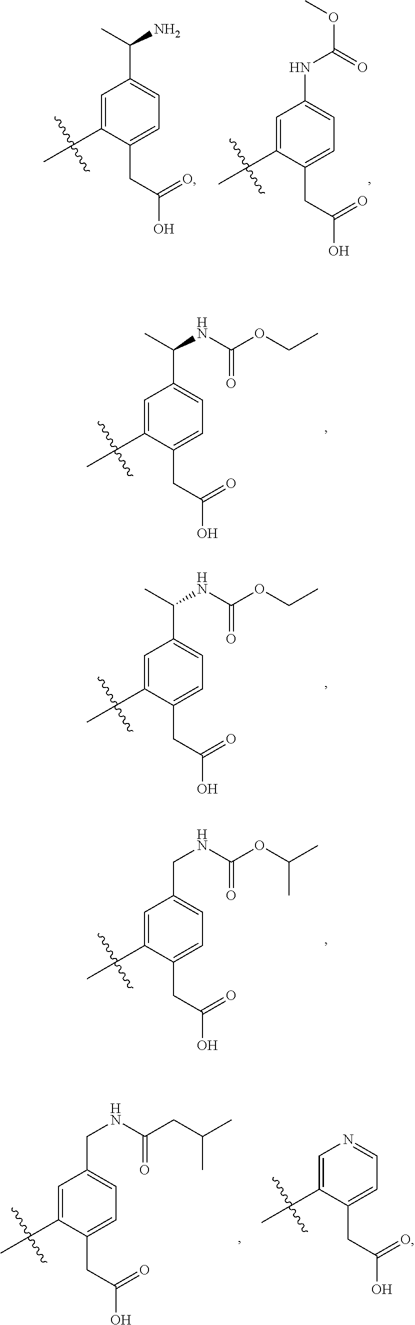

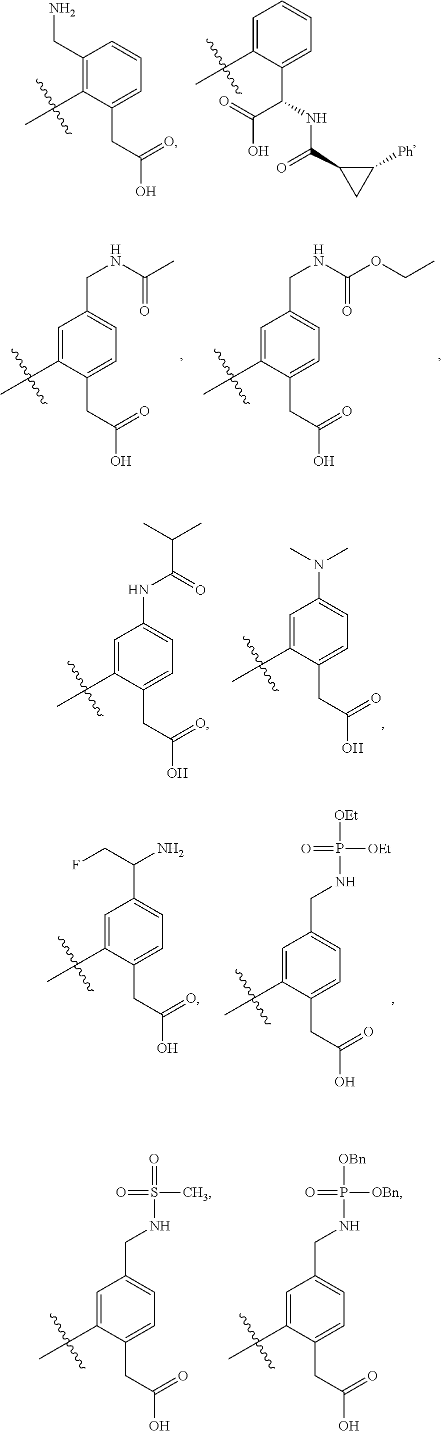

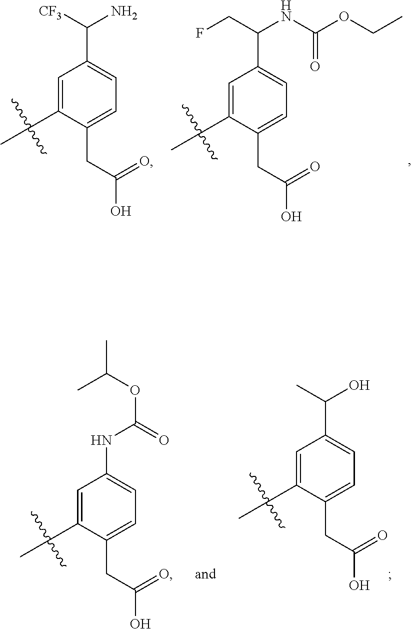 [SMILES: *c1cc(C(N)C(F)(F)F)ccc1CC(=O)O ; *c1cc(C(CF)NC(=O)OCC)ccc1CC(=O)O ; *c1cc(NC(=O)OC(C)C)ccc1CC(=O)O ; *c1cc(C(C)O)ccc1CC(=O)O]

\textcircled{B} is selected from the group consisting of

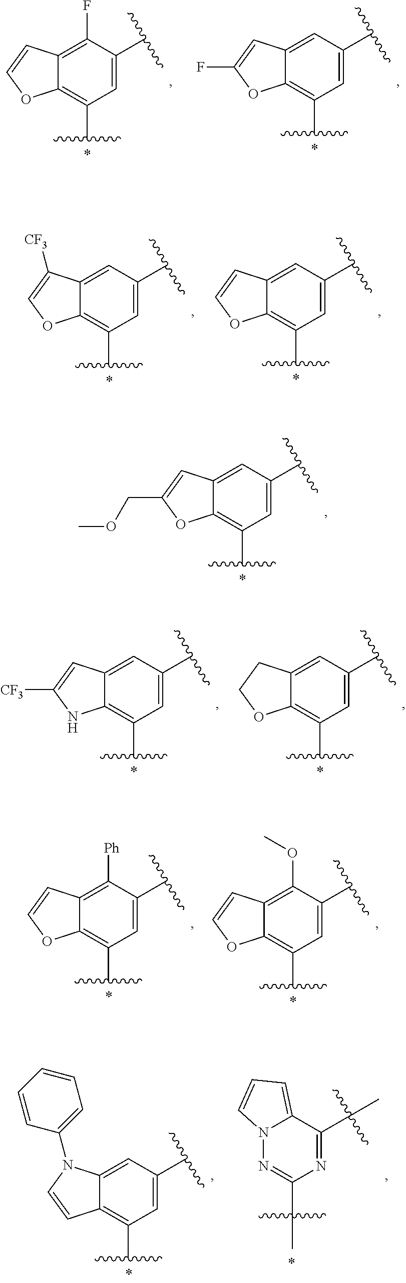

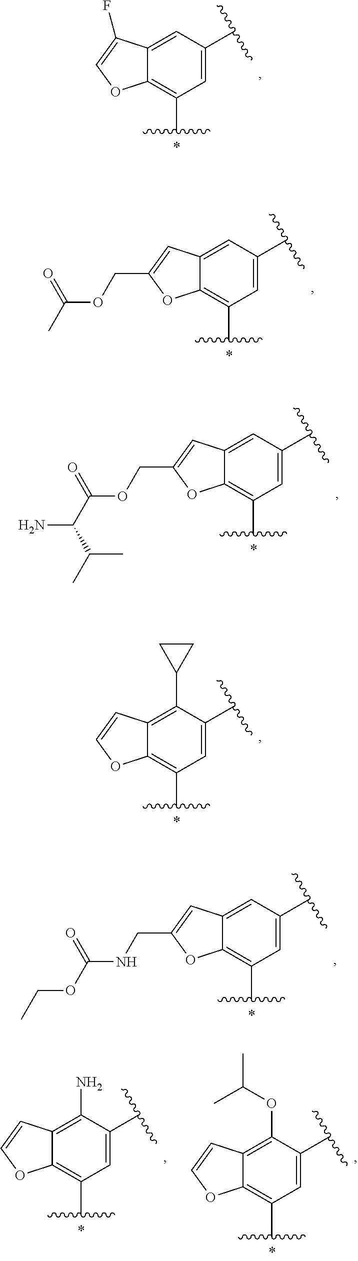

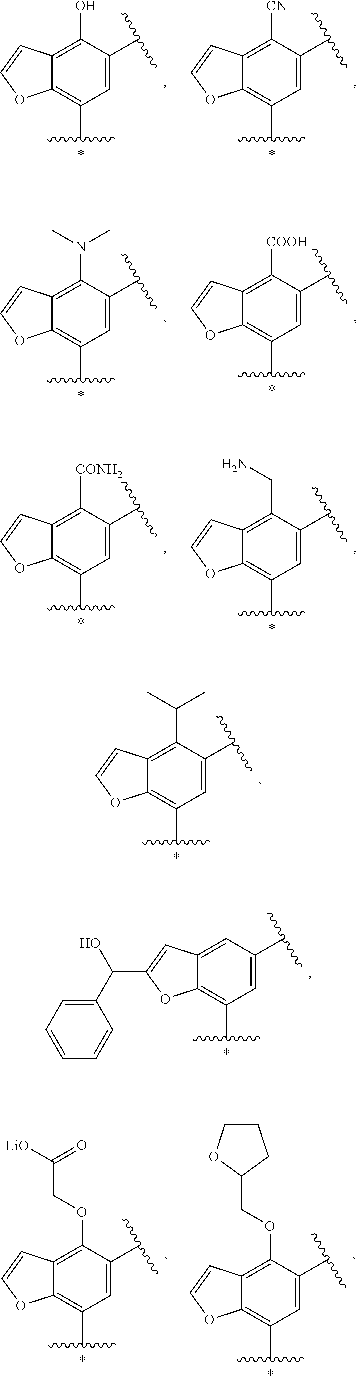

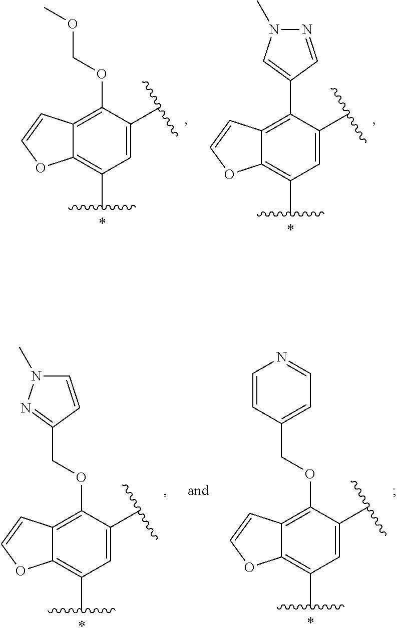 [SMILES: *c1cc(*)c2occc2c1OCOC ; *c1cc(*)c2occc2c1-c1cnn(C)c1 ; *c1cc(*)c2occc2c1OCc1ccn(C)n1 ; *c1cc(*)c2occc2c1OCc1ccncc1]

wherein the asterisk (*) indicates the point of attachment to \textcircled{C};

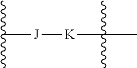

is selected from the group consisting of

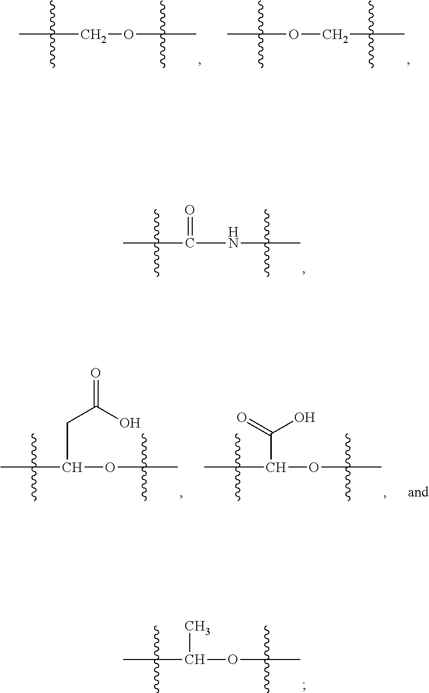

and
\textcircled{C} is selected from the group consisting of

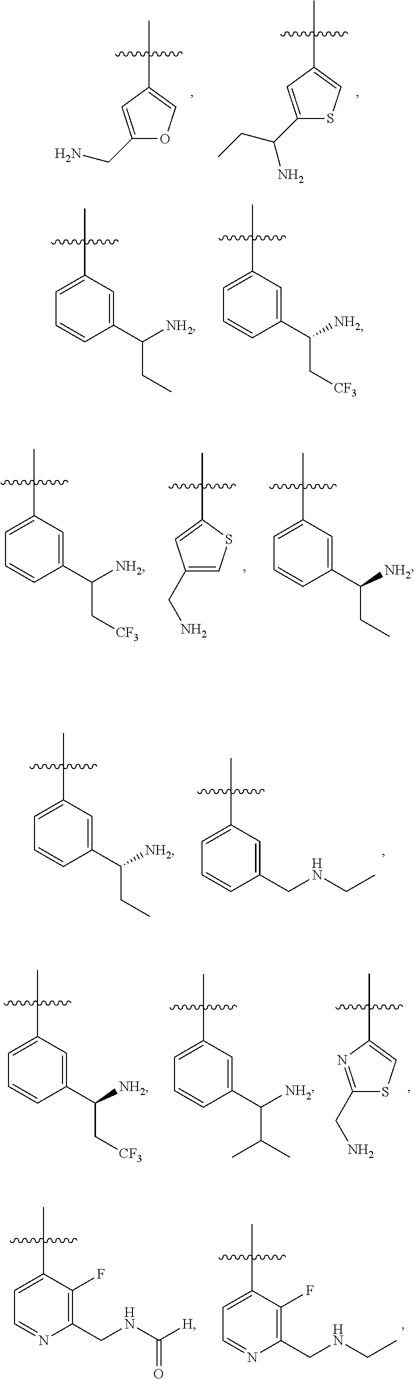

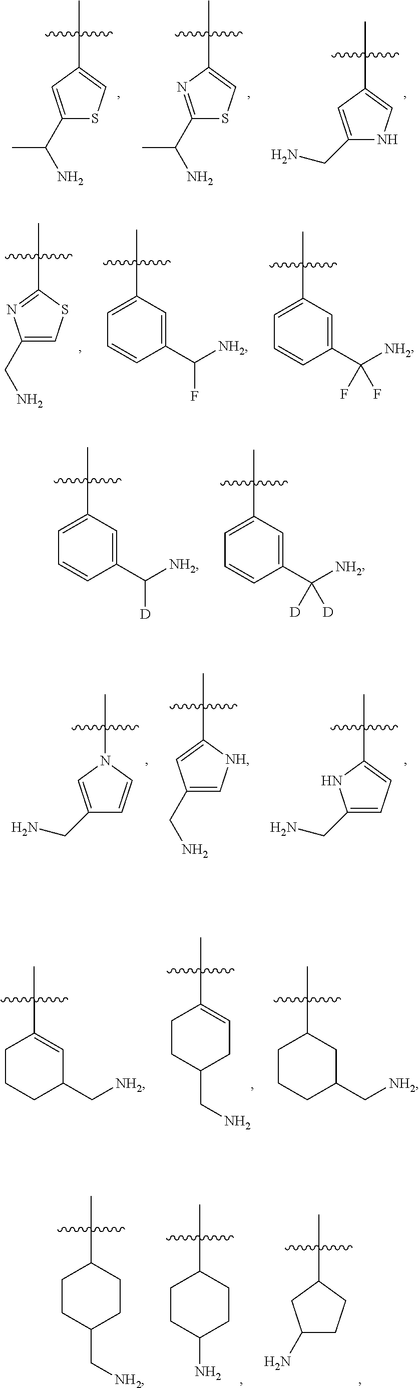

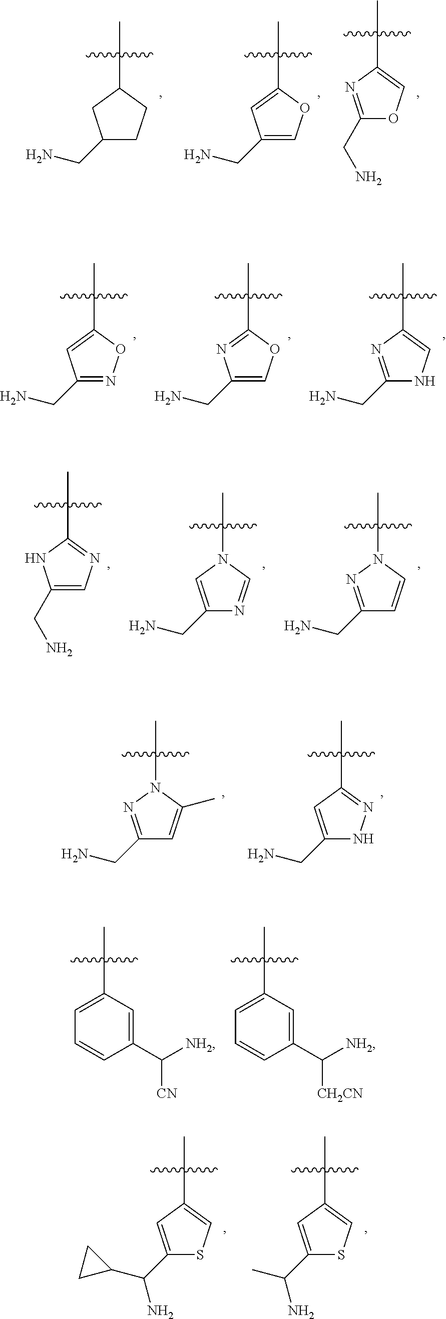

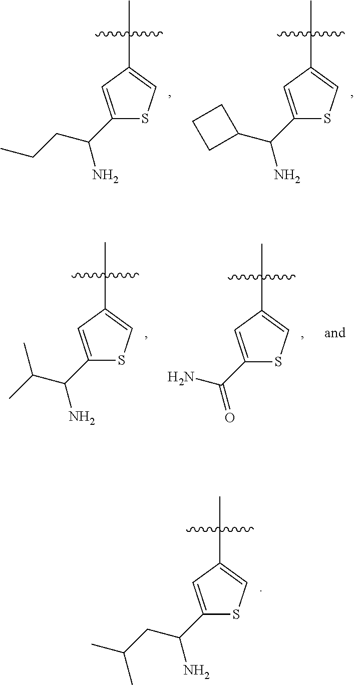 [SMILES: *c1csc(C(N)CCC)c1 ; *c1csc(C(N)C2CCC2)c1 ; *c1csc(C(N)C(C)C)c1 ; *c1csc(C(N)=O)c1 ; *c1csc(C(N)CC(C)C)c1]

In some embodiments of the compounds of formula (III), \textcircled{A} is

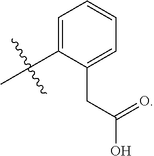 [SMILES: *c1ccccc1CC(=O)O]

In some embodiments of the compounds of formula (III), \textcircled{B} is

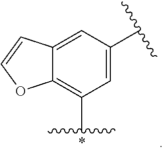 [SMILES: *c1cc(*)c2occc2c1]

In some embodiments of the compounds of formula (III),

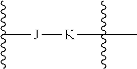

is

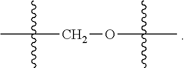 [SMILES: *CO*]

The present invention provides compounds having the structure of Formula (IV), and pharmaceutically acceptable salts thereof:

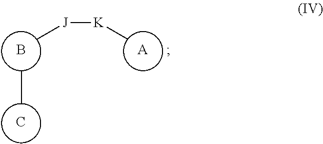

wherein:
\textcircled{A} is selected from the group consisting of

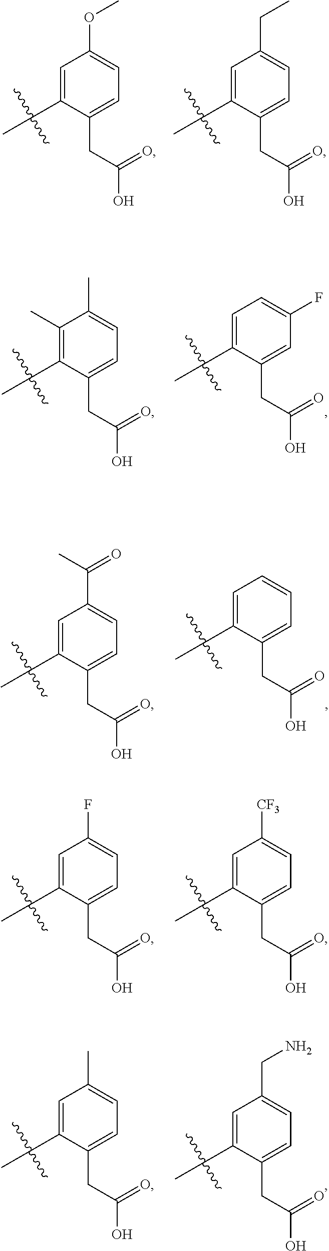

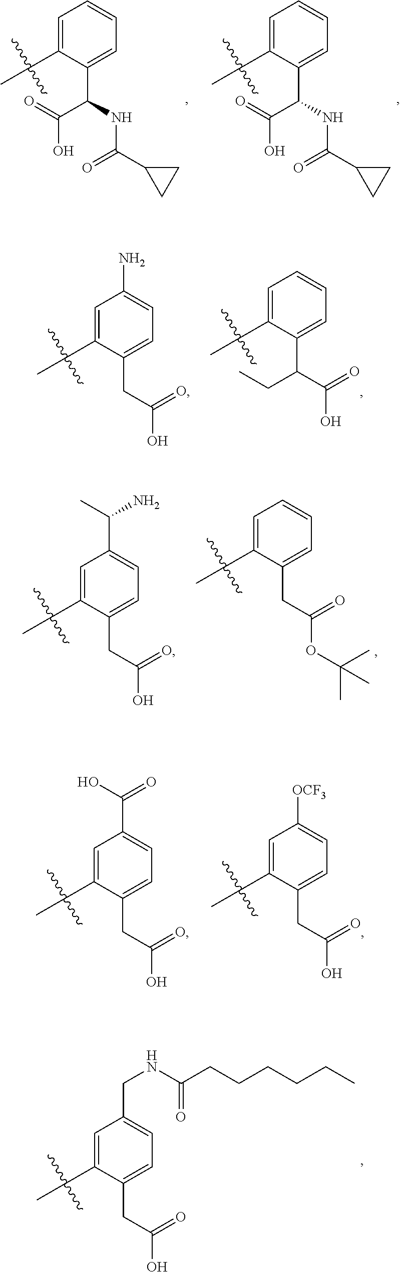

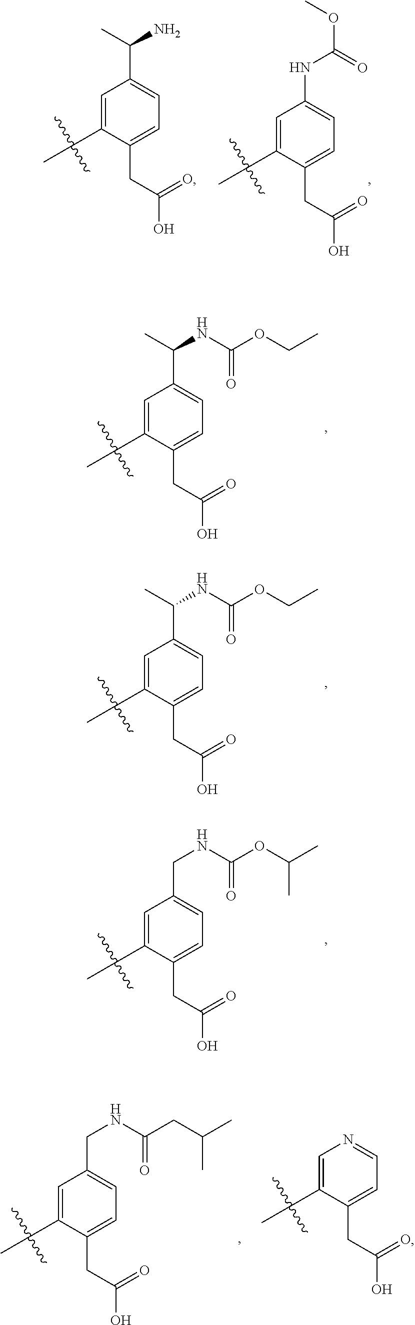

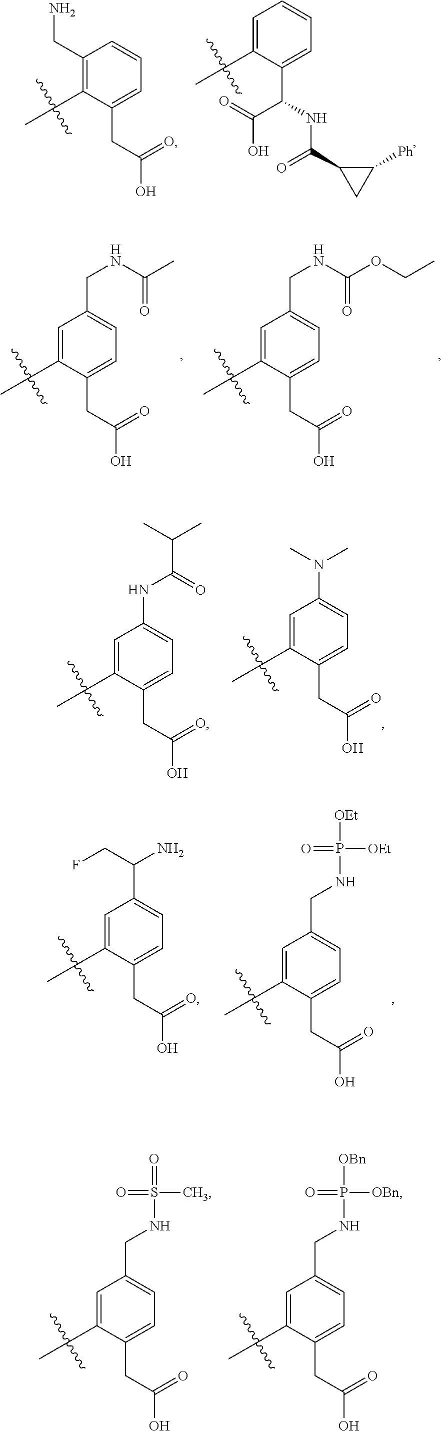

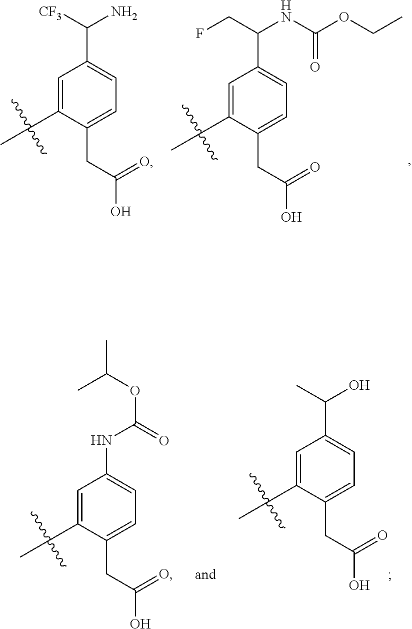 [SMILES: *c1cc(C(N)C(F)(F)F)ccc1CC(=O)O ; *c1cc(C(CF)NC(=O)OCC)ccc1CC(=O)O ; *c1cc(NC(=O)OC(C)C)ccc1CC(=O)O ; *c1cc(C(C)O)ccc1CC(=O)O]

\textcircled{B} is selected from the group consisting of

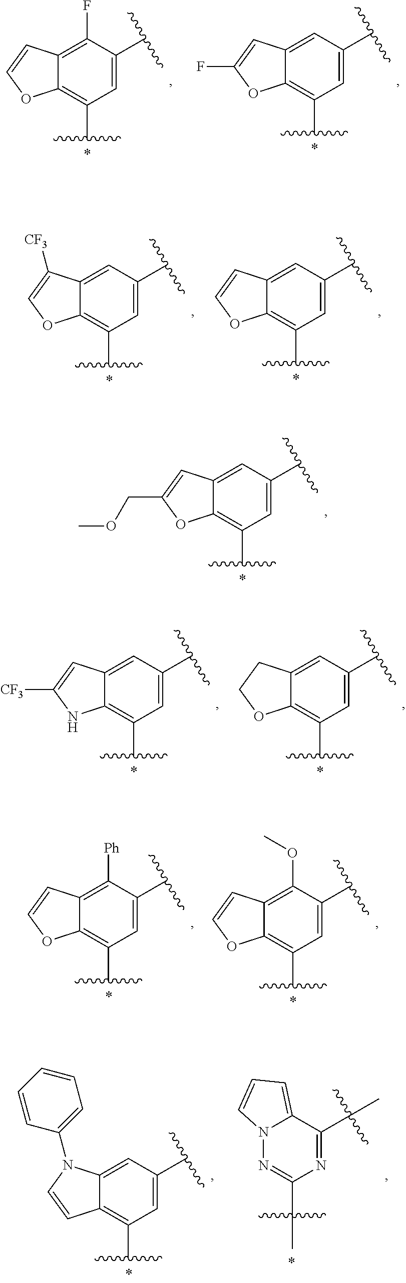

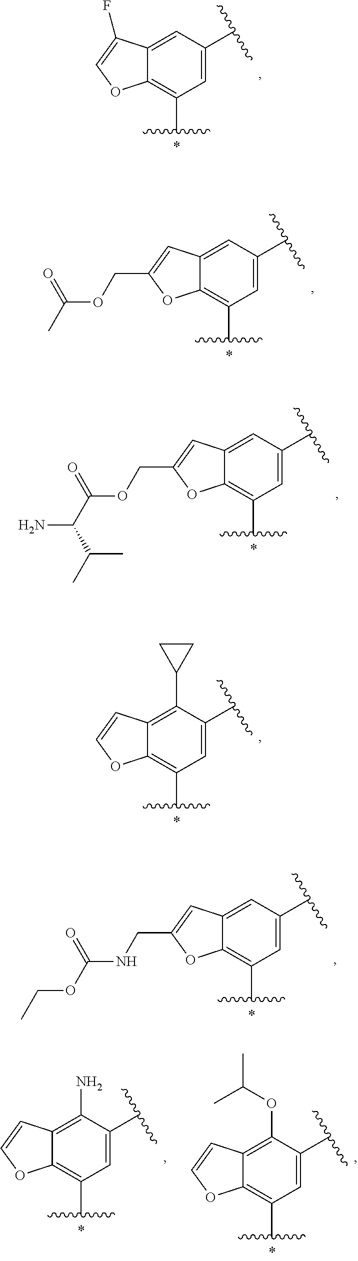

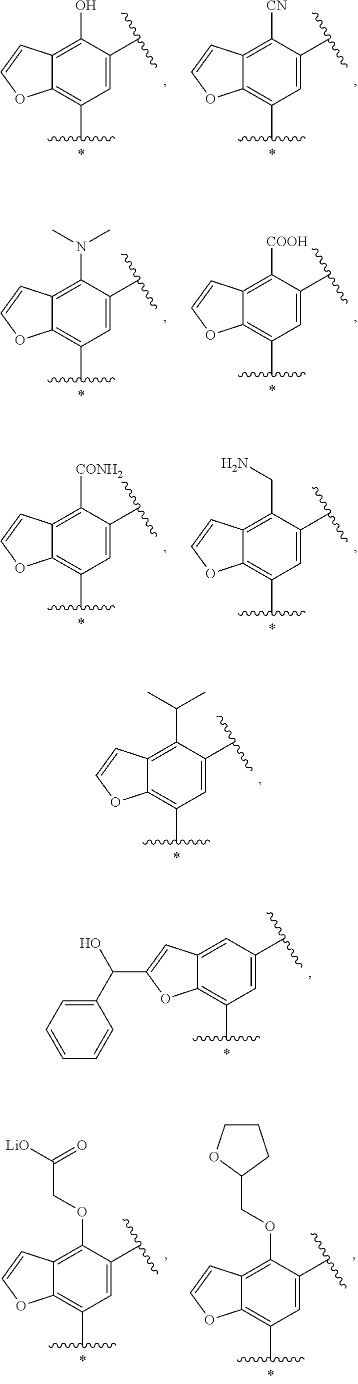

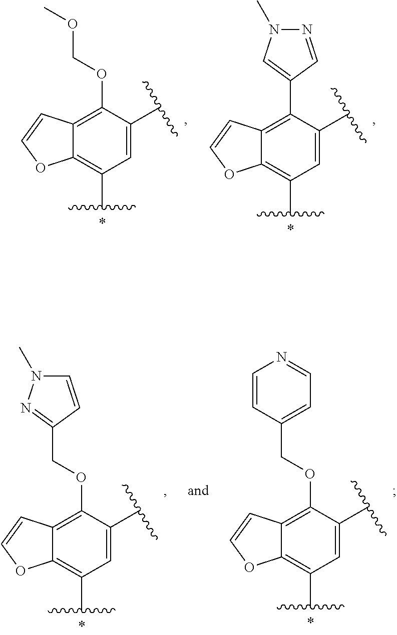 [SMILES: *c1cc(*)c2occc2c1OCOC ; *c1cc(*)c2occc2c1-c1cnn(C)c1 ; *c1cc(*)c2occc2c1OCc1ccn(C)n1 ; *c1cc(*)c2occc2c1OCc1ccncc1]

wherein the asterisk (*) indicates the point of attachment to \textcircled{C};

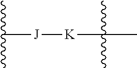

is selected from the group consisting of

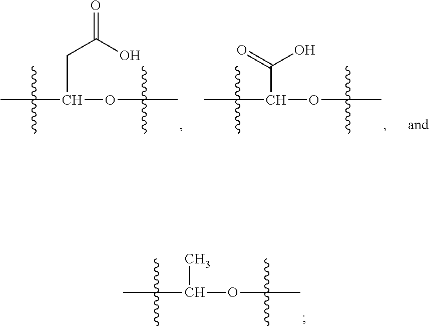

and
\textcircled{C} is selected from the group consisting of

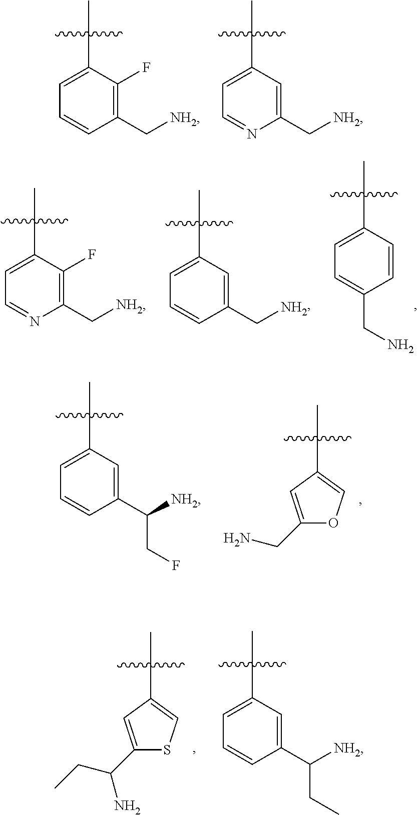

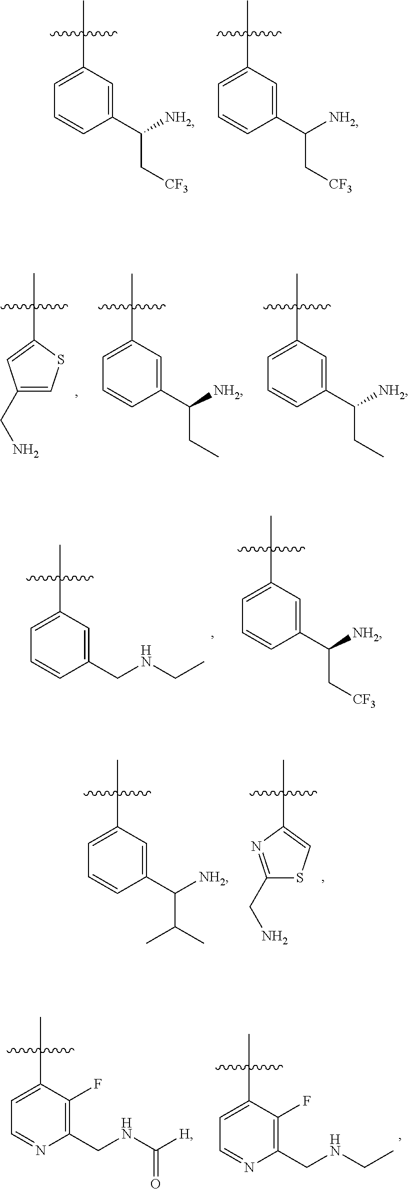

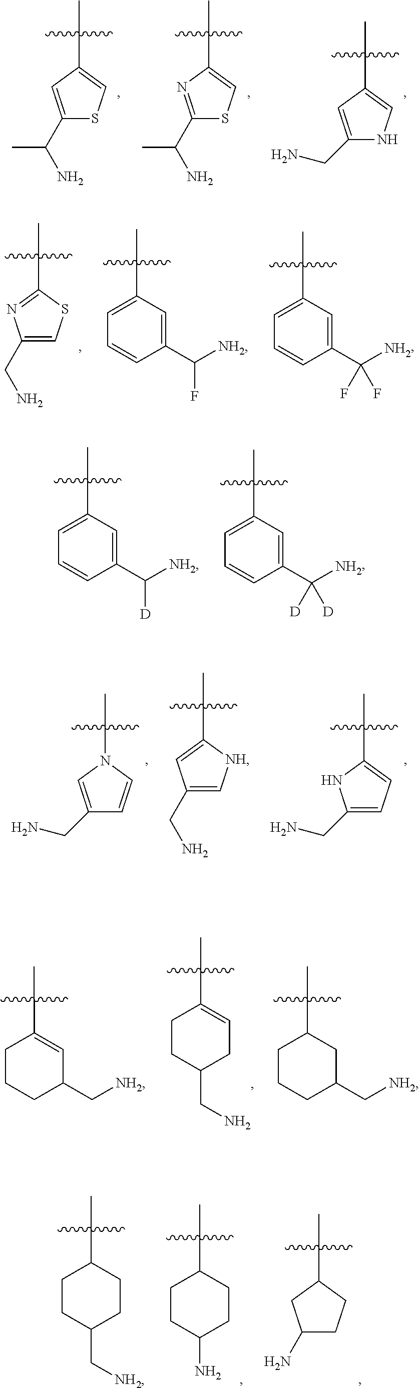

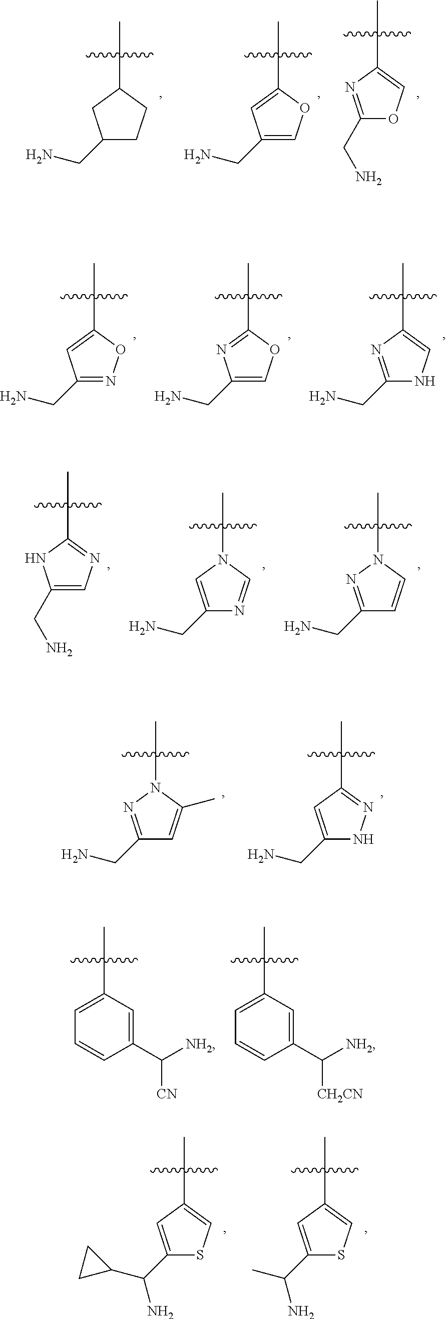

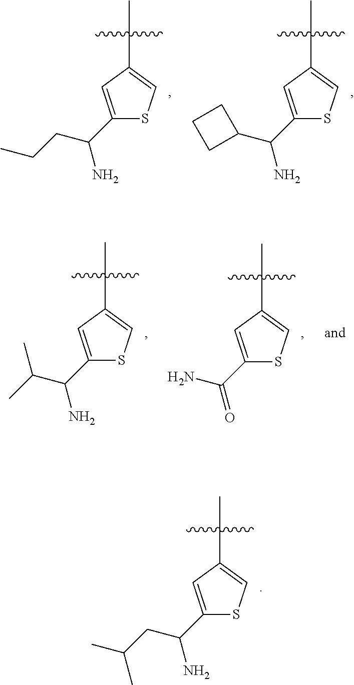 [SMILES: *c1csc(C(N)CCC)c1 ; *c1csc(C(N)C2CCC2)c1 ; *c1csc(C(N)C(C)C)c1 ; *c1csc(C(N)=O)c1 ; *c1csc(C(N)CC(C)C)c1]

In some embodiments of the compounds of formula (IV), \textcircled{A} is

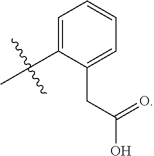 [SMILES: *c1ccccc1CC(=O)O]

In some embodiments of the compounds of formula (IV), \textcircled{B} is

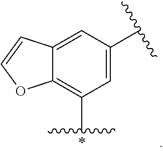 [SMILES: *c1cc(*)c2occc2c1]

In some embodiments of the compounds of formula (IV), \textcircled{C} is selected from the group consisting of

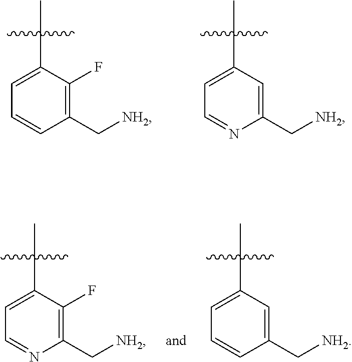 [SMILES: *c1cccc(CN)c1F ; *c1ccnc(CN)c1 ; *c1ccnc(CN)c1F ; *c1cccc(CN)c1]

In preferred embodiments, \textcircled{C} is

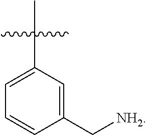 [SMILES: *c1cccc(CN)c1]

In certain embodiments, the compound of the invention is selected from the following table of compounds, and pharmaceutically acceptable salts thereof:

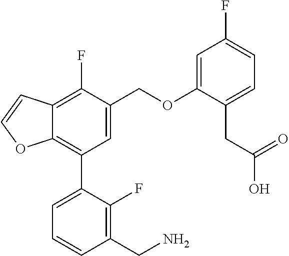

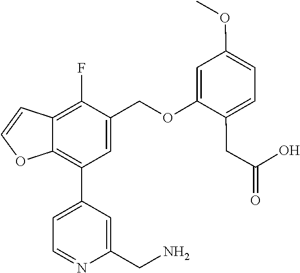

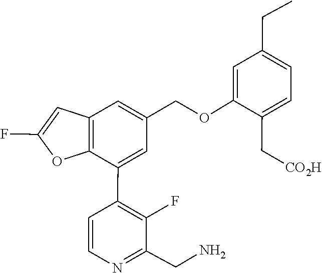

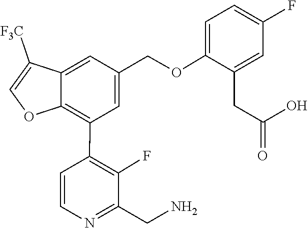

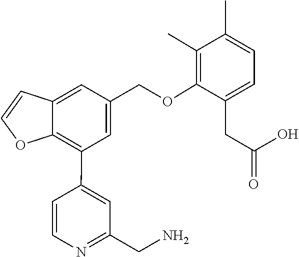

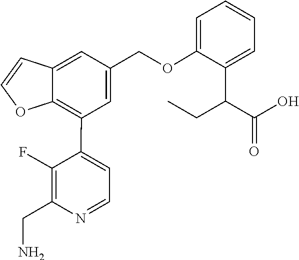

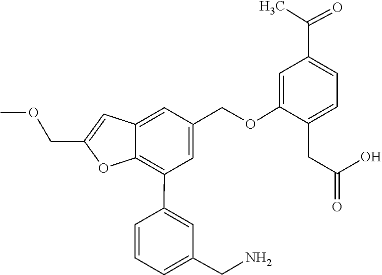

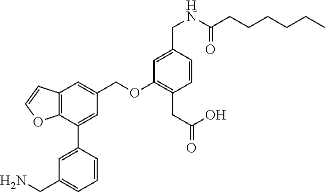

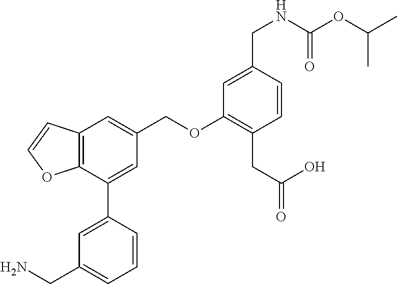

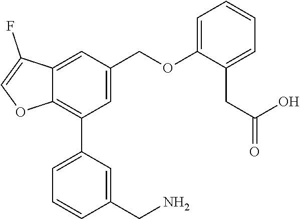

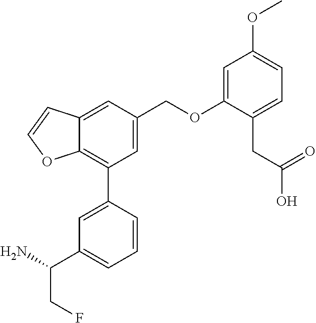

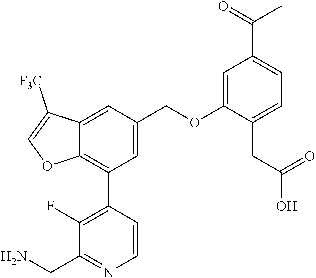

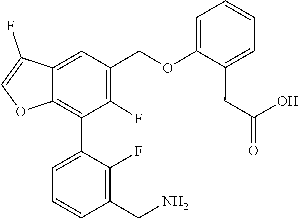

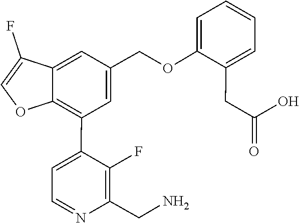

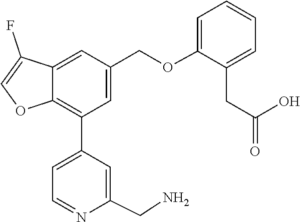

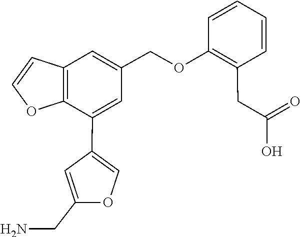

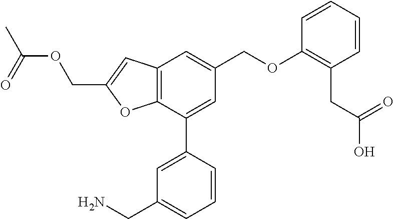

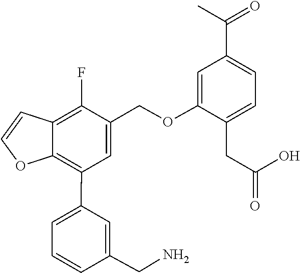

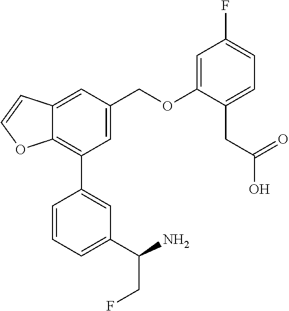

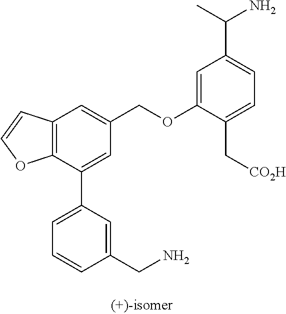

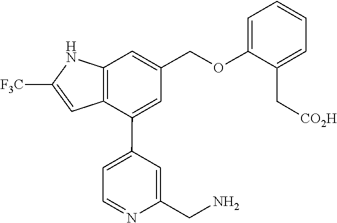

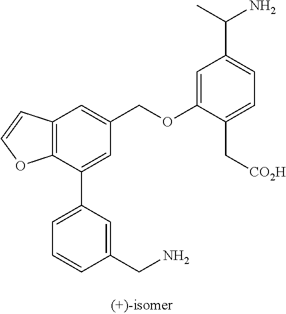

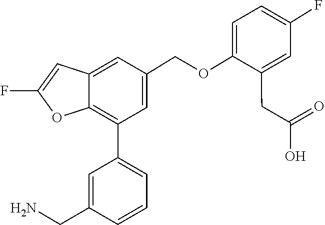

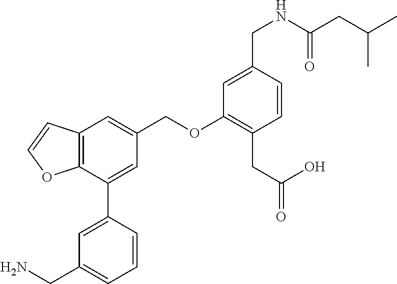

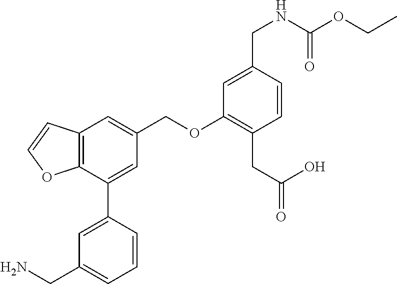

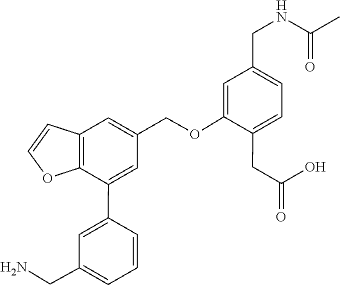

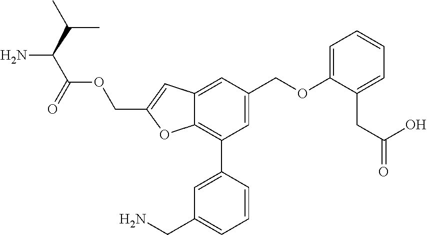

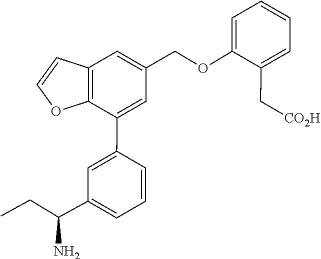

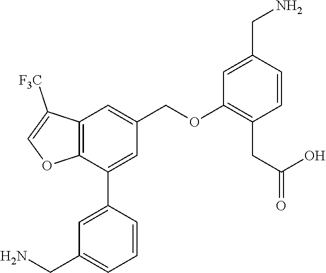

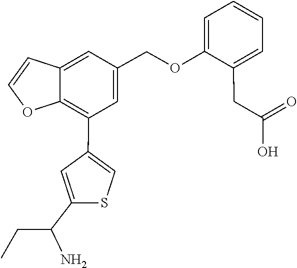

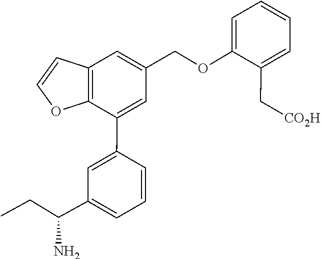

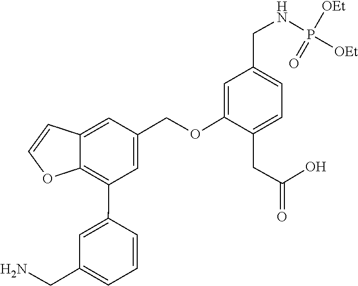

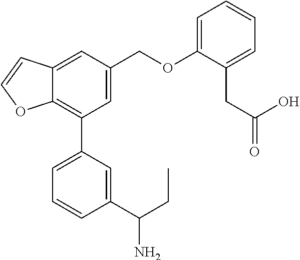

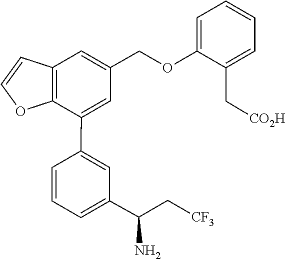

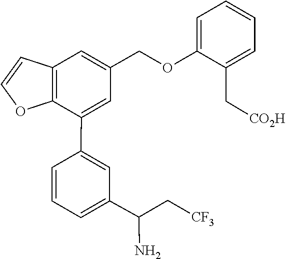

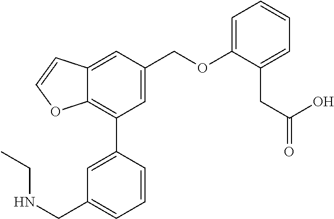

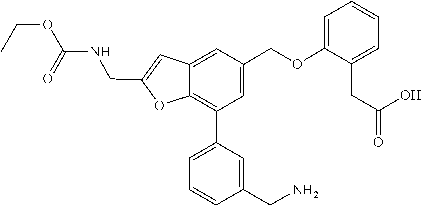

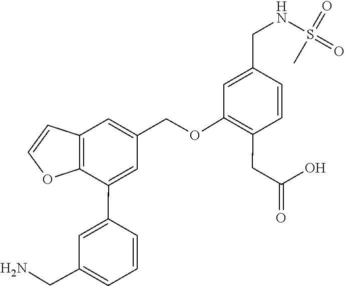

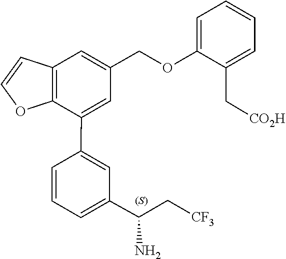

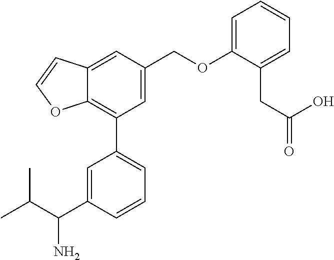

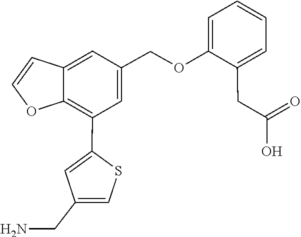

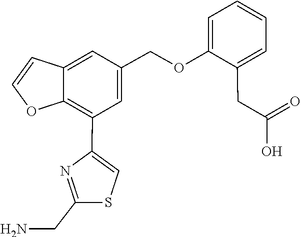

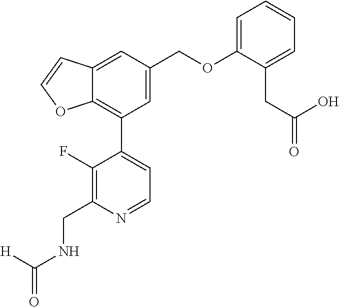

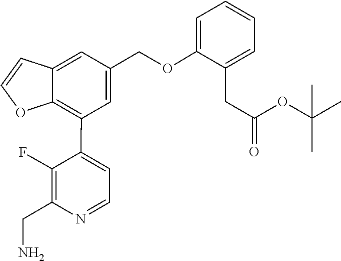

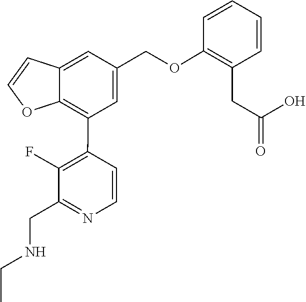

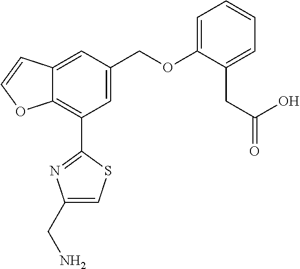

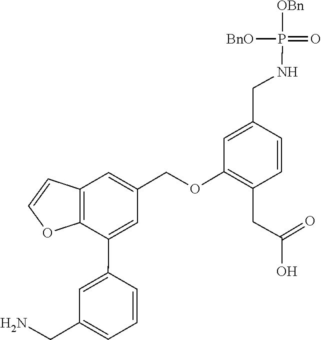

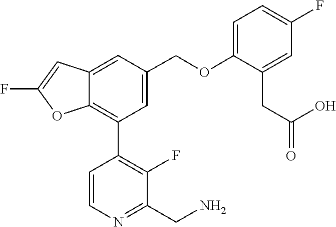

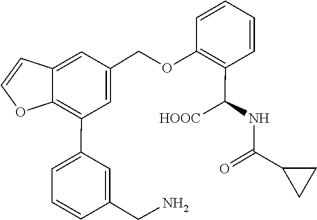

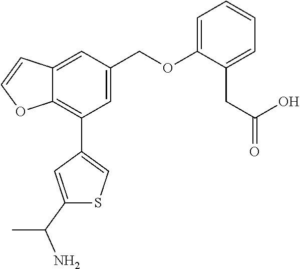

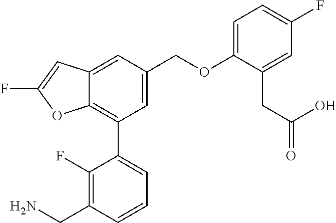

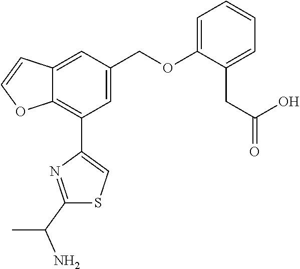

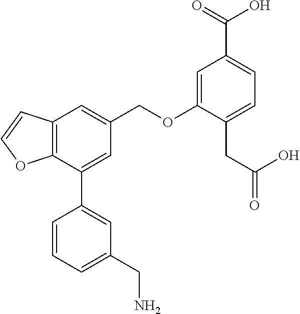

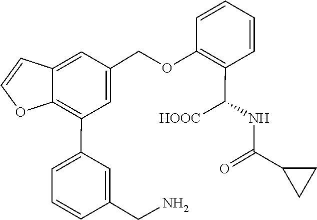

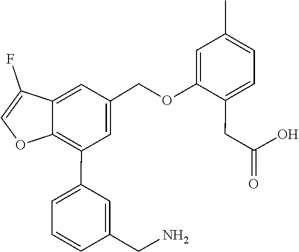

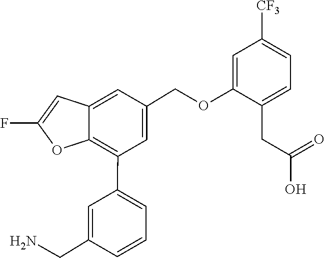

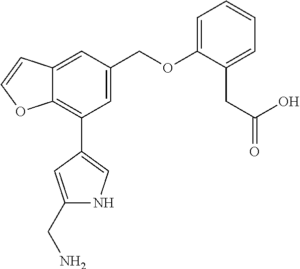

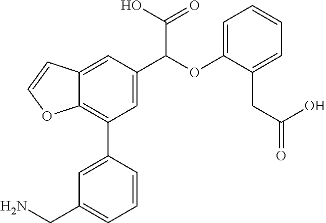

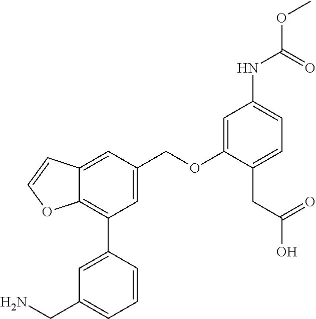

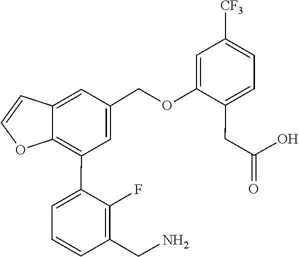

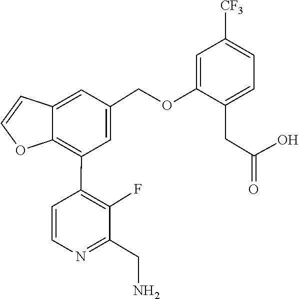

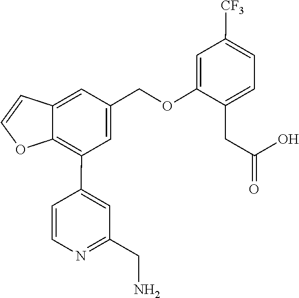

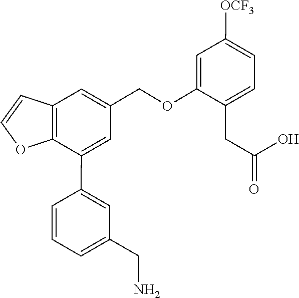

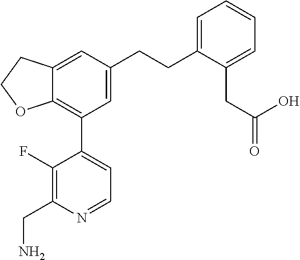

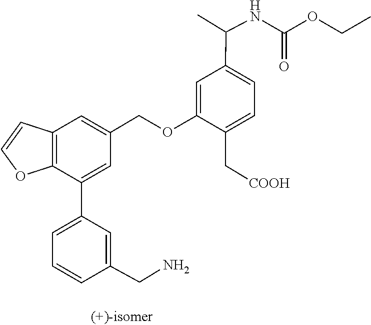

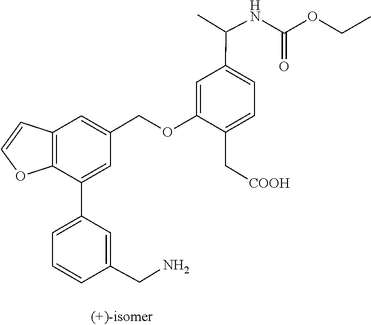

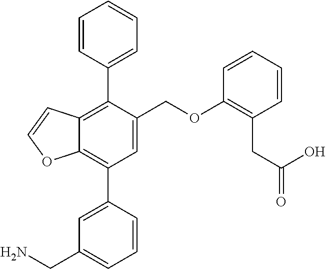

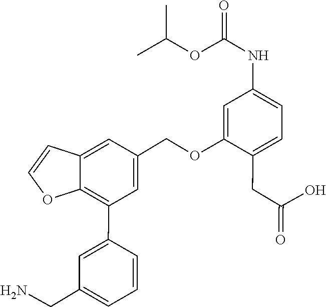

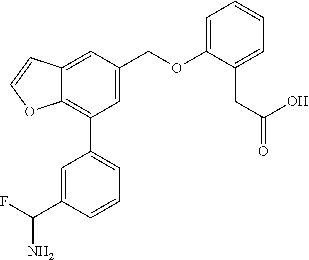

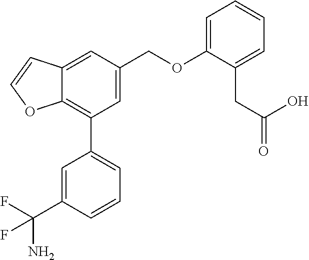

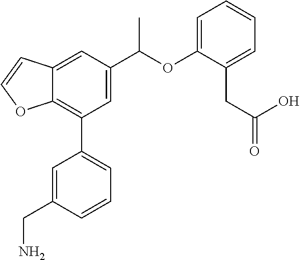

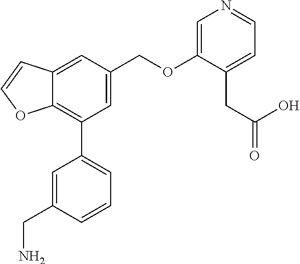

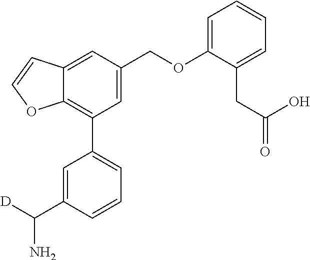

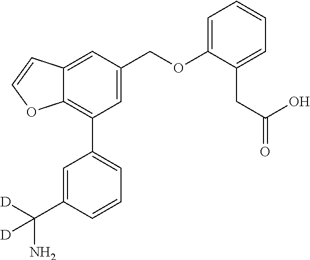

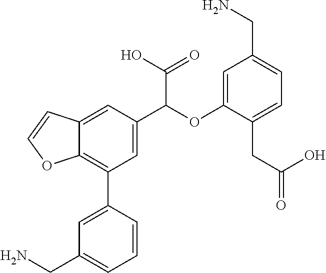

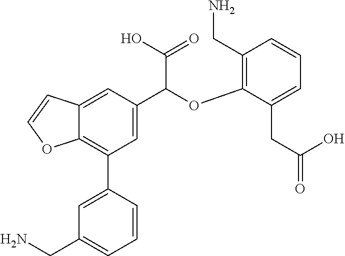

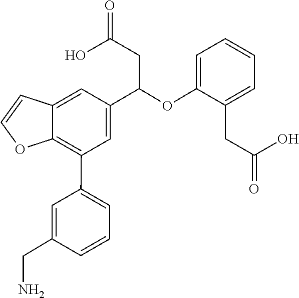

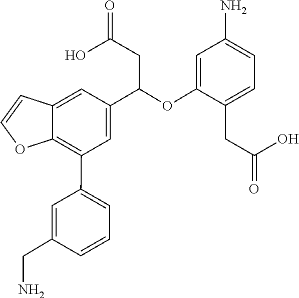

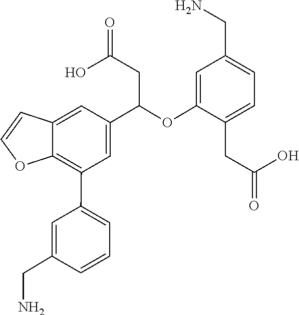

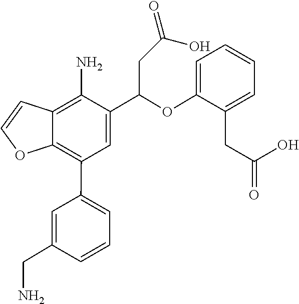

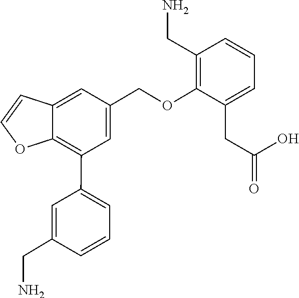

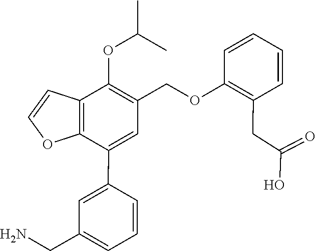

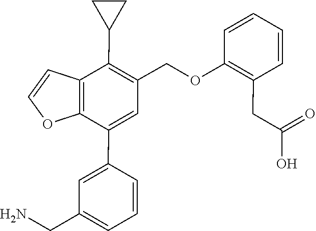

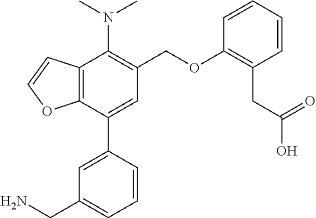

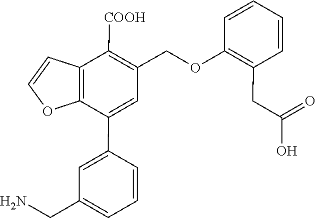

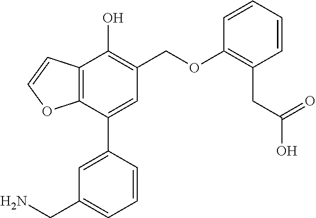

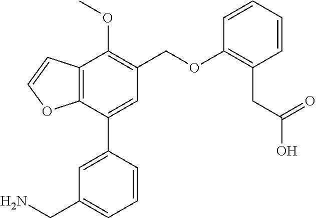

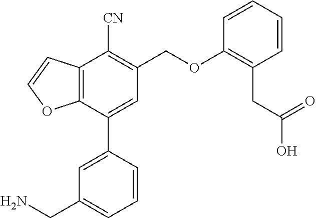

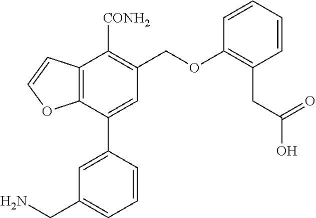

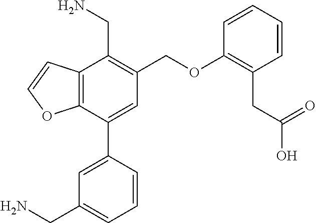

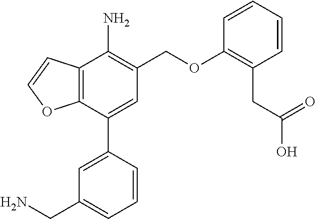

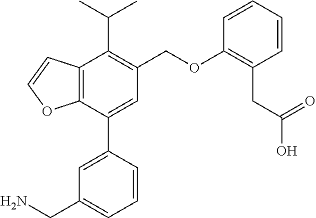

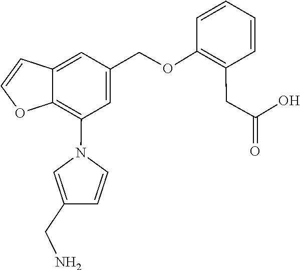

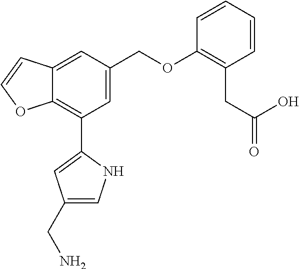

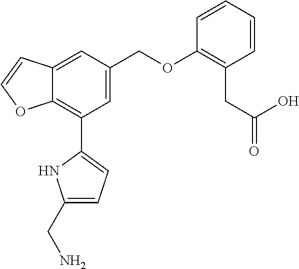

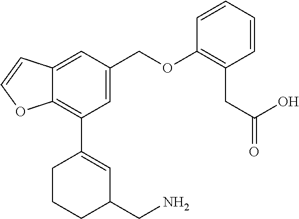

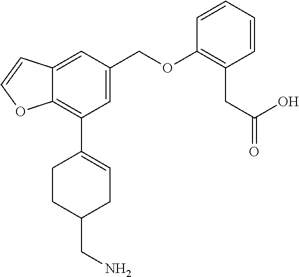

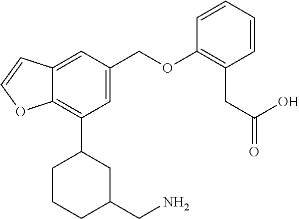

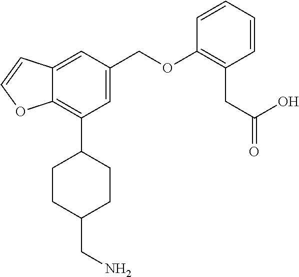

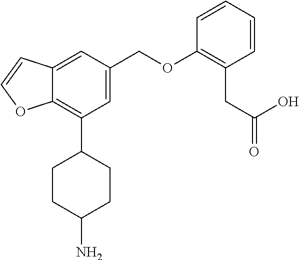

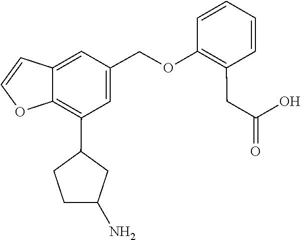

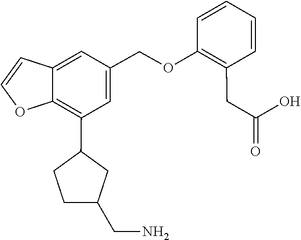

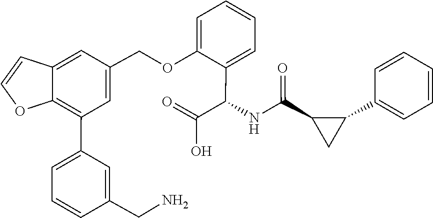

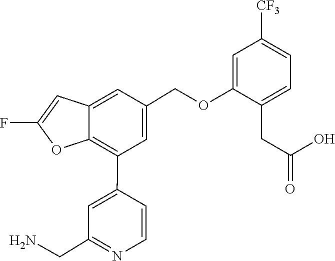

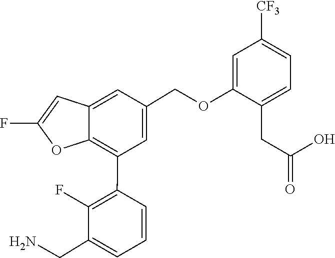

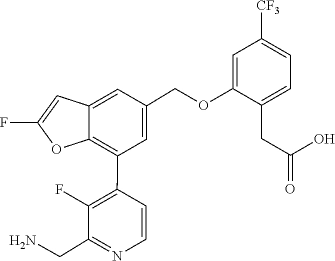

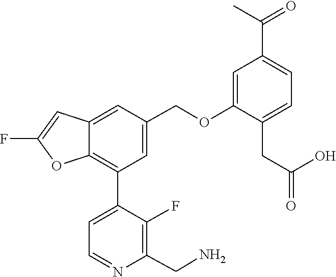

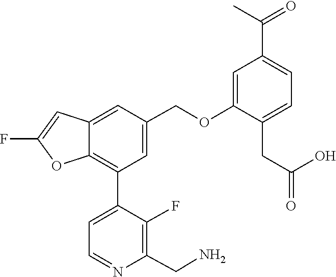

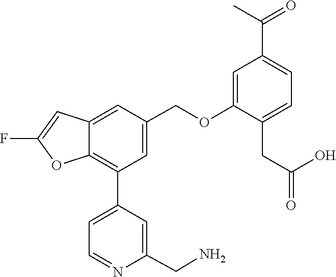

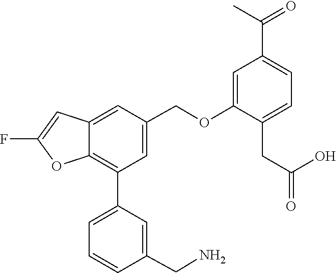

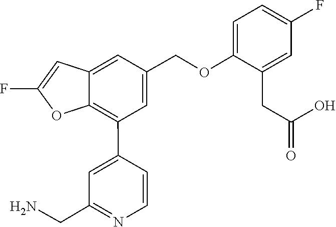

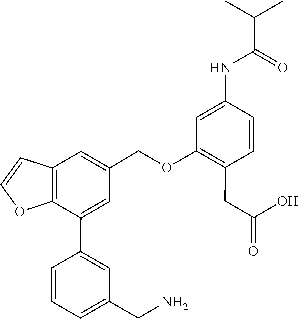

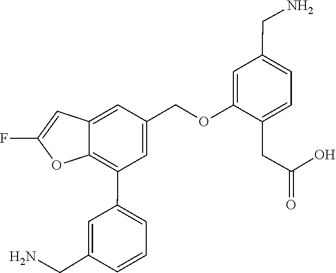

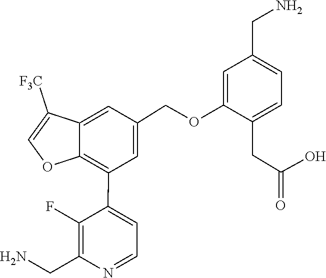

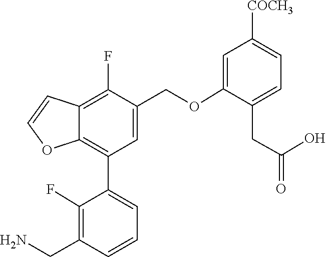

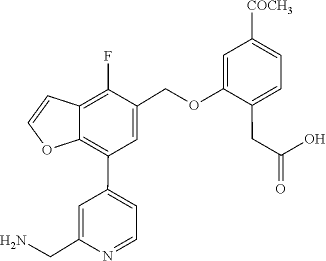

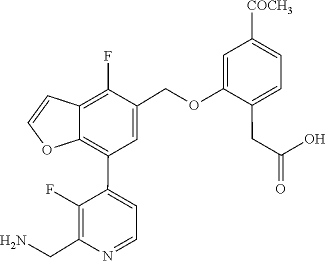

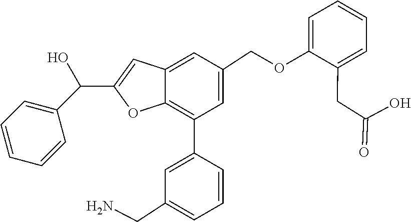

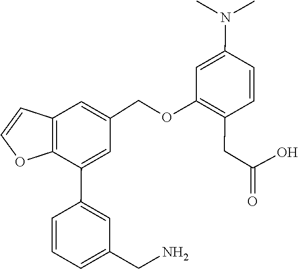

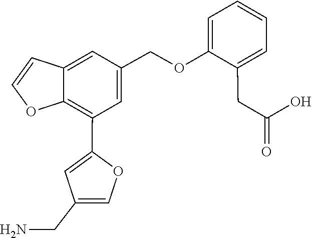

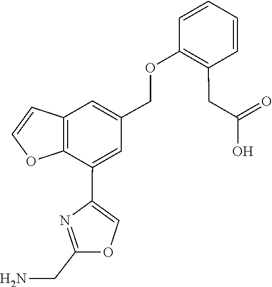

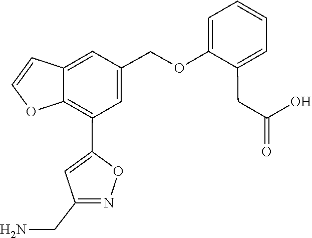

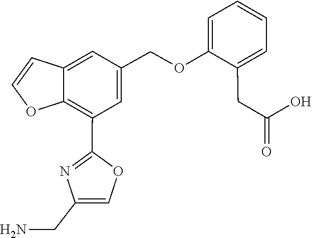

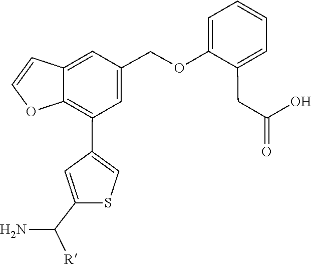

wherein R′ is methyl, propyl,
isopropyl, isobutyl,
cyclopropyl, or cyclobutyl

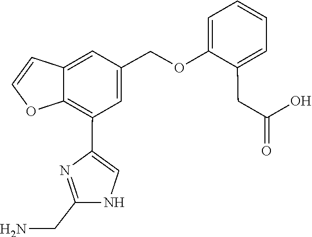 [SMILES: NCc1nc(-c2cc(COc3ccccc3CC(=O)O)cc3ccoc23)c[nH]1]

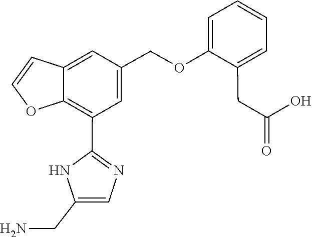 [SMILES: NCc1cnc(-c2cc(COc3ccccc3CC(=O)O)cc3ccoc23)[nH]1]

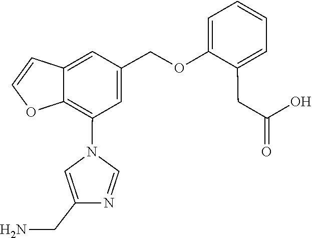 [SMILES: NCc1cn(-c2cc(COc3ccccc3CC(=O)O)cc3ccoc23)cn1]

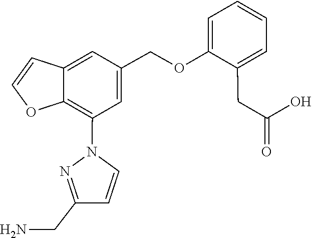 [SMILES: NCc1ccn(-c2cc(COc3ccccc3CC(=O)O)cc3ccoc23)n1]

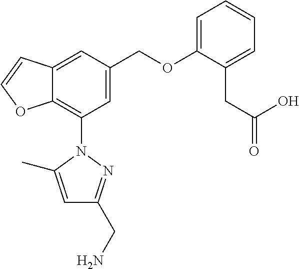 [SMILES: Cc1cc(CN)nn1-c1cc(COc2ccccc2CC(=O)O)cc2ccoc12]

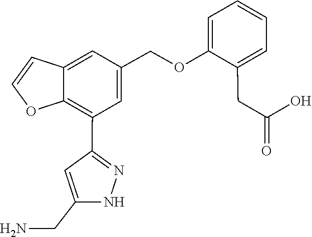 [SMILES: NCc1cc(-c2cc(COc3ccccc3CC(=O)O)cc3ccoc23)n[nH]1]

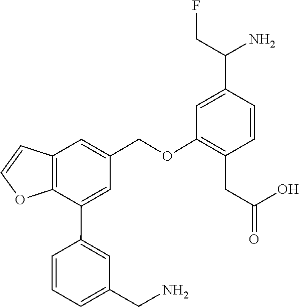 [SMILES: NCc1cccc(-c2cc(COc3cc(C(N)CF)ccc3CC(=O)O)cc3ccoc23)c1]

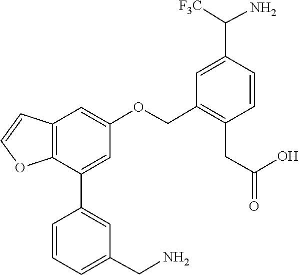 [SMILES: NCc1cccc(-c2cc(OCc3cc(C(N)C(F)(F)F)ccc3CC(=O)O)cc3ccoc23)c1]

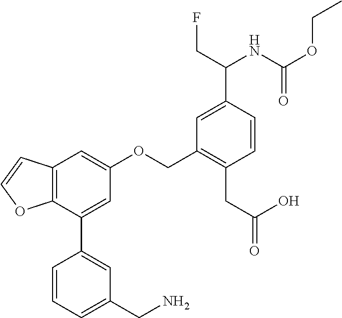 [SMILES: CCOC(=O)NC(CF)c1ccc(CC(=O)O)c(COc2cc(-c3cccc(CN)c3)c3occc3c2)c1]

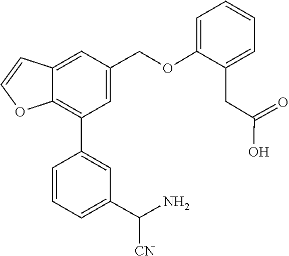 [SMILES: N#CC(N)c1cccc(-c2cc(COc3ccccc3CC(=O)O)cc3ccoc23)c1]

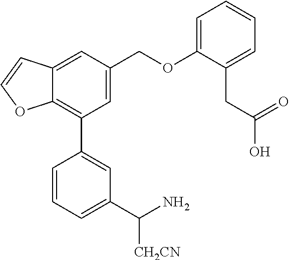

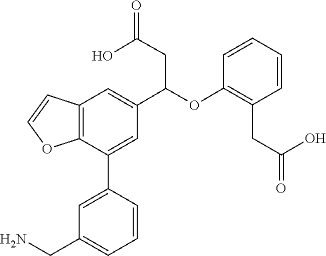 [SMILES: NCc1cccc(-c2cc(C(CC(=O)O)Oc3ccccc3CC(=O)O)cc3ccoc23)c1]

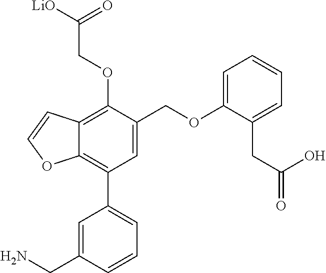

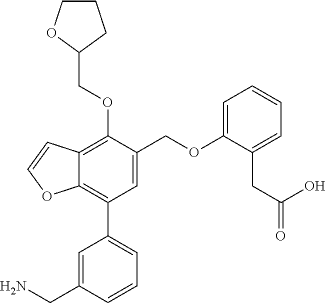 [SMILES: NCc1cccc(-c2cc(COc3ccccc3CC(=O)O)c(OCC3CCCO3)c3ccoc23)c1]

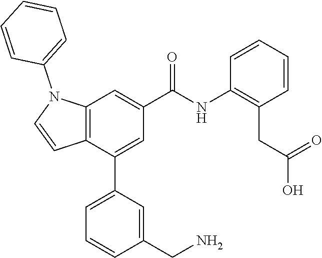 [SMILES: NCc1cccc(-c2cc(C(=O)Nc3ccccc3CC(=O)O)cc3c2ccn3-c2ccccc2)c1]

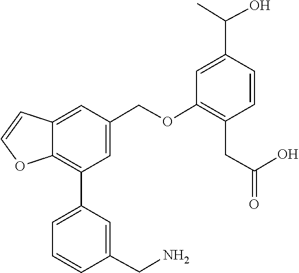 [SMILES: CC(O)c1ccc(CC(=O)O)c(OCc2cc(-c3cccc(CN)c3)c3occc3c2)c1]

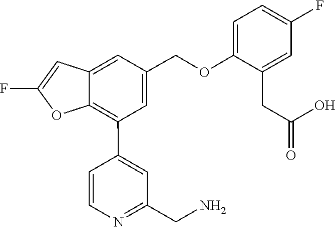 [SMILES: NCc1cc(-c2cc(COc3ccc(F)cc3CC(=O)O)cc3cc(F)oc23)ccn1]

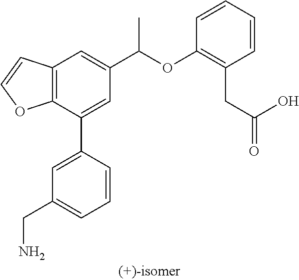 [SMILES: CC(Oc1ccccc1CC(=O)O)c1cc(-c2cccc(CN)c2)c2occc2c1]

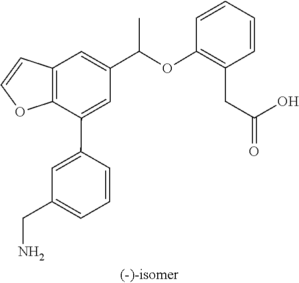 [SMILES: CC(Oc1ccccc1CC(=O)O)c1cc(-c2cccc(CN)c2)c2occc2c1]

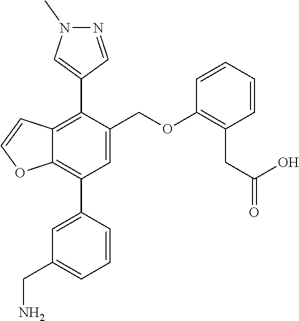 [SMILES: Cn1cc(-c2c(COc3ccccc3CC(=O)O)cc(-c3cccc(CN)c3)c3occc23)cn1]

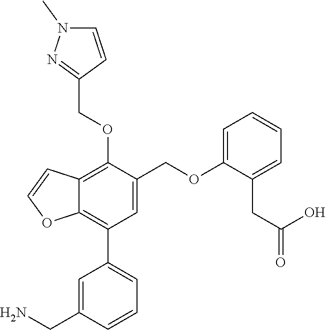 [SMILES: Cn1ccc(COc2c(COc3ccccc3CC(=O)O)cc(-c3cccc(CN)c3)c3occc23)n1]

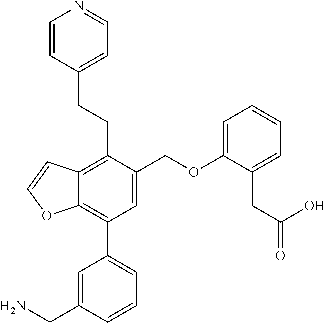 [SMILES: NCc1cccc(-c2cc(COc3ccccc3CC(=O)O)c(CCc3ccncc3)c3ccoc23)c1]

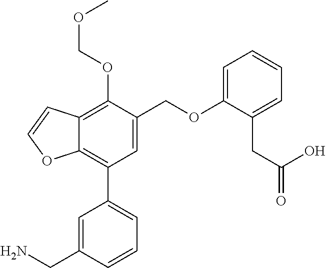 [SMILES: COCOc1c(COc2ccccc2CC(=O)O)cc(-c2cccc(CN)c2)c2occc12]

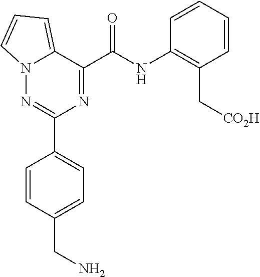 [SMILES: NCc1ccc(-c2nc(C(=O)Nc3ccccc3CC(=O)O)c3cccn3n2)cc1]

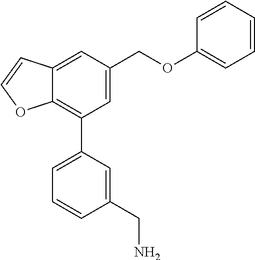 [SMILES: NCc1cccc(-c2cc(COc3ccccc3)cc3ccoc23)c1]

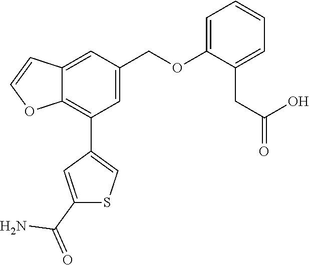 [SMILES: NC(=O)c1cc(-c2cc(COc3ccccc3CC(=O)O)cc3ccoc23)cs1]

### Pharmaceutical Compositions

The invention provides pharmaceutical compositions, each comprising one or more compounds of the invention, or pharmaceutically acceptable salts thereof, and a pharmaceutically acceptable carrier. In certain embodiments, the pharmaceutical composition comprises a compound of the invention, or a pharmaceutically acceptable salt thereof, and a pharmaceutically acceptable carrier. In certain embodiments, the pharmaceutical composition comprises a plurality of compounds of the invention, which may include pharmaceutically acceptable salts thereof, and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of a disease or condition characterized by aberrant complement system activity.

Pharmaceutical compositions of the invention can be prepared by combining one or more compounds of the invention with a pharmaceutically acceptable carrier and, optionally, one or more additional pharmaceutically active agents.

### Methods of Use

The present invention provides compounds, and pharmaceutically acceptable salts thereof, that are useful for treating or preventing a disease or condition characterized by aberrant complement system activity.

In certain aspects, the invention provides a compound of the invention, or a pharmaceutically acceptable salt thereof, for use as a medicament.

In certain aspects, the invention provides methods of treating or preventing a disease or condition characterized by aberrant complement system activity. The method includes the step of administering to a subject in need thereof a therapeutically effective amount of a compound of the invention, or a pharmaceutically acceptable salt thereof, thereby treating or preventing the disease or condition characterized by aberrant complement system activity. By reducing complement system activity in the subject, the disease or condition characterized by aberrant complement system activity is treated.

Alternatively, in certain aspects, the invention provides a compound of the invention, or a pharmaceutically acceptable salt thereof, for treatment of a disease or condition characterized by aberrant complement system activity.

Alternatively, in certain aspects, the invention provides the use of a compound of the invention, or a pharmaceutically acceptable salt thereof, for the manufacture of a medicament for use in treatment of a disease or condition characterized by aberrant complement system activity.

As used herein, a “disease or condition characterized by aberrant complement system activity” refers to any disease or condition in which it is desirable to reduce complement system activity. For example, it may be desirable to reduce complement system activity in the setting of inappropriate activation or hyperactivation of the complement system.

In certain embodiments, the disease or condition characterized by aberrant complement system activity is an immunological disorder.

In certain embodiments, the disease or condition characterized by aberrant complement system activity is a disease of the central nervous system.

In certain embodiments, the disease or condition characterized by aberrant complement system activity is a renal disease.

In certain embodiments, the disease or condition characterized by aberrant complement system activity is a cardiovascular disease.

In certain embodiments, the disease or condition characterized by aberrant complement system activity is a neurodegenerative disease or neurological disease

In certain embodiments, the disease or condition characterized by aberrant complement system activity is selected from the group consisting of paroxysmal nocturnal hemoglobinuria, atypical hemolytic uremic syndrome, organ transplant rejection, myasthenia gravis, neuromyelitis optica, membranoproliferative glomerulonephritis, dense-deposit disease, cold agglutinin disease, and catastrophic antiphospholipid syndrome.

In certain embodiments, the disease or condition is paroxysmal nocturnal hemoglobinuria.

In certain embodiments, the disease or condition is atypical hemolytic uremic syndrome.

In certain embodiments, the disease or condition is organ transplant rejection.

In certain embodiments, the disease or condition is myasthenia gravis.

In certain embodiments, the disease or condition is neuromyelitis optica.

In certain embodiments, the disease or condition is membranoproliferative glomerulonephritis.

In certain embodiments, the disease or condition is dense-deposit disease.

In certain embodiments, the disease or condition is cold agglutinin disease.

In certain embodiments, the disease or condition is catastrophic antiphospholipid syndrome.

In other embodiments, the disease or condition characterized by aberrant complement system activity is adult respiratory distress syndrome, myocardial infarct, lung inflammation, hyperacute rejection (transplantation rejection), sepsis, cardiopulmonary bypass, burns, asthma, restenosis, multiple organ dysfunction syndrome, Guillain-Barre syndrome, hemorrhagic shock, paroxysmal nocturnal hemoglobinuria, glomerulonephritis, systemic lupus erythematosus, rheumatoid arthritis, infertility, Alzheimer's disease, organ rejection (transplantation), myasthenia gravis, multiple sclerosis, platelet storage, or hemodialysis.

In other embodiments, the disease or condition characterized by aberrant complement system activity is selected from the group consisting of antineutrophil cytoplasmic antibody (ANCA)-associated vasculitis (AAV), warm autoimmune hemolytic anemia, IgA nephropathy, C3 glomerulonephritis, and focal segmental glomerulosclerosis.

In certain embodiments, the disease or condition characterized by aberrant complement system activity is a hematological disorder.

In other embodiments, the disease or condition characterized by aberrant complement system activity is an ocular disorder or an eye disorder.

In certain embodiments, the disease or condition characterized by aberrant complement system activity is macular degeneration, age-related macular degeneration (AMD), macular edema, diabetic macular edema, choroidal neovascularization (CNV), uveitis, Behcet's uveitis, proliferative diabetic retinopathy, non-proliferative diabetic retinopathy, glaucoma, hypertensive retinopathy, a corneal neovascularization disease, post-corneal transplant rejection, a corneal dystrophic disease, an autoimmune dry eye disease, Stevens-Johnson syndrome, Sjogren's syndrome, an environmental dry eye disease, Fuchs' endothelial dystrophy, retinal vein occlusion, or post-operative inflammation.

### Formulations, Routes of Administration, and Dosing

The compounds of the invention, and pharmaceutically acceptable salts thereof, can be formulated as pharmaceutical compositions and administered to a mammalian host, such as a human patient, in a variety of forms adapted to the chosen route of administration, e.g., orally or parenterally, by intravenous, intraperitoneal, intramuscular, topical, or subcutaneous routes. Additional routes of administration are also contemplated by the invention.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2% to about 60% of the weight of a given unit dosage form. The amount of active compound in such therapeutically useful compositions is such that an effective dosage level will be obtained.

The tablets, troches, pills, capsules, and the like may also contain the following diluents and carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water or physiologically acceptable aqueous solution, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are known in the art; for example, see Jacquet et al. (U.S. Pat. No. 4,608,392; incorporated herein by reference), Geria (U.S. Pat. No. 4,992,478; incorporated herein by reference), Smith et al. (U.S. Pat. No. 4,559,157; incorporated herein by reference), and Wortzman (U.S. Pat. No. 4,820,508; incorporated herein by reference).

Useful dosages of the compounds of the invention can be determined, at least initially, by comparing their in vitro activity and in vivo activity in animal models. Methods for the extrapolation of effective dosages in mice, and other animals, to humans are known in the art; for example, see U.S. Pat. No. 4,938,949 (incorporated herein by reference).

The amount of the compound, or pharmaceutically acceptable salt thereof, required for use in treatment will vary not only with the particular compound or salt selected but also with the route of administration, the nature of the condition being treated, and the age and condition of the patient and will be ultimately at the discretion of the attendant physician or clinician.

In general, however, a suitable dose will be in the range of from about 0.5 to about 100 mg/kg body weight of the recipient per day, e.g., from about 3 to about 90 mg/kg of body weight per day, from about 6 to about 75 mg per kilogram of body weight per day, from about of 10 to about 60 mg/kg of body weight per day, or from about 15 to about 50 mg/kg of body weight per day.

Compounds of the invention, or pharmaceutically acceptable salts thereof, can be conveniently formulated in unit dosage form; for example, containing 5 to 1000 mg, 10 to 750 mg, or 50 to 500 mg of active ingredient per unit dosage form. In one embodiment, the invention provides a composition comprising a compound of the invention, or pharmaceutically acceptable salts thereof, formulated in such a unit dosage form. The desired dose may conveniently be presented in a single dose or as divided doses to be administered at appropriate intervals, for example, as two, three, four or more sub-doses per day. The sub-dose itself may be further divided, e.g., into a number of discrete loosely spaced administrations.

Compounds of the invention, or pharmaceutically acceptable salts thereof, can also be administered in combination with other therapeutic agents, for example, other agents that are useful for treating or preventing ischemia, blood loss, or reperfusion injury. In certain embodiments, compounds of the invention, and pharmaceutically acceptable salts thereof, can also be administered in combination with one or more other therapeutic agents that are useful for treating or preventing an ocular disorder or eye disorder.

Other delivery systems can include time-release, delayed release, or sustained release delivery systems such as are well-known in the art. Such systems can avoid repeated administrations of the active compound, increasing convenience to the subject and the physician. Many types of release delivery systems are available and known to those of ordinary skill in the art. Use of a long-term sustained release implant may be desirable. Long-term release, as used herein, means that the delivery system or is implant constructed and arranged to deliver therapeutic levels of the active ingredient for at least 30 days, and preferably 60 days.

In certain embodiments, a compound of the invention is formulated for intraocular administration, for example direct injection or insertion within or in association with an intraocular medical device. In certain embodiments, a compound of the invention is formulated as an ophthalmic solution. In certain embodiments, a compound of the invention can be administered via ocular delivery, for example, by local ocular administration, including topical, intravitreal, periocular, transscleral, retrobulbar, juxtascleral, suprachoroidal, or sub-tenon administration. A compound of the invention can be administered via ocular delivery either alone or in combination with one or more additional therapeutic agents.

The compounds of the invention may be formulated for depositing into a medical device, which may include any of a variety of conventional grafts, stents, including stent grafts, catheters, balloons, baskets, or other device that can be deployed or permanently implanted within a body lumen. As a particular example, it would be desirable to have devices and methods which can deliver compounds of the invention to the region of a body which has been treated by interventional technique.

In exemplary embodiment, a compound of the invention may be deposited within a medical device, such as a stent, and delivered to the treatment site for treatment of a portion of the body.

Stents have been used as delivery vehicles for therapeutic agents (i.e., drugs). Intravascular stents are generally permanently implanted in coronary or peripheral vessels. Stent designs include those of U.S. Pat. No. 4,733,655 (Palmaz), U.S. Pat. No. 4,800,882 (Gianturco), or U.S. Pat. No. 4,886,062 (Wiktor). Such designs include both metal and polymeric stents, as well as self-expanding and balloon-expandable stents. Stents may also be used to deliver a drug at the site of contact with the vasculature, as disclosed in U.S. Pat. No. 5,102,417 (Palmaz), U.S. Pat. No. 5,419,760 (Narciso, Jr.), U.S. Pat. No. 5,429,634 (Narciso, Jr.), and in International Patent Application Nos. WO 91/12779 (Medtronic, Inc.) and WO 90/13332 (Cedars-Sanai Medical Center), for example.

The term “deposited” means that the compound is coated, adsorbed, placed, or otherwise incorporated into the device by methods known in the art. For example, the compound may be embedded and released from within (“matrix type”) or surrounded by and released through (“reservoir type”) polymer materials that coat or span the medical device. In the latter example, the compound may be entrapped within the polymer materials or coupled to the polymer materials using one or more the techniques for generating such materials known in the art. In other formulations, the compound may be linked to the surface of the medical device without the need for a coating, for example by means of detachable bonds, and release with time or can be removed by active mechanical or chemical processes. In other formulations, the compound may be in a permanently immobilized form that presents the compound at the implantation site.

In certain embodiments, the compound may be incorporated with polymer compositions during the formation of biocompatible coatings for medical devices, such as stents. The coatings produced from these components are typically homogeneous and are useful for coating a number of devices designed for implantation.

The polymer may be either a biostable or a bioabsorbable polymer depending on the desired rate of release or the desired degree of polymer stability, but frequently a bioabsorbable polymer is preferred for this embodiment since, unlike a biostable polymer, it will not be present long after implantation to cause any adverse, chronic local response. Bioabsorbable polymers that could be used include, but are not limited to, poly(L-lactic acid), polycaprolactone, polyglycolide (PGA), poly(lactide-co-glycolide) (PLLA/PGA), poly(hydroxybutyrate), poly(hydroxybutyrate-co-valerate), polydioxanone, polyorthoester, polyanhydride, poly(glycolic acid), poly(D-lactic acid), poly(L-lactic acid), poly(D, L-lactic acid), poly(D, L-lactide) (PLA), poly (L-lactide) (PLLA), poly(glycolic acid-co-trimethylene carbonate) (PGA/PTMC), polyethylene oxide (PEO), polydioxanone (PDS), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid, polyepsilon caprolactone, polyhydroxy butyric acid, polyorthoesters, polyacetals, polydihydropyrans, polycyanoacrylates, cross linked or amphipathic block copolymers of hydrogels, and other suitable bioabsorbable poplymers known in the art. Also, biostable polymers with a relatively low chronic tissue response such as polyurethanes, silicones, and polyesters could be used, and other polymers could also be used if they can be dissolved and cured or polymerized on the medical device such as polyolefins, polyisobutylene and ethylene-alphaolefin copolymers; acrylic polymers and copolymers, vinyl halide polymers and copolymers, such as polyvinyl chloride; polyvinylpyrrolidone; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; pyran copolymer; polyhydroxy-propyl-methacrylamide-phenol; polyhydroxyethyl-aspartamide-phenol; polyethyleneoxide-polylysine substituted with palmitoyl residues; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Polymers and semipermeable polymer matrices may be formed into shaped articles, such as valves, stents, tubing, prostheses and the like.

In certain embodiments of the invention, the compound of the invention is coupled to a polymer or semipermeable polymer matrix that is formed as a stent or stent-graft device.

Typically, polymers are applied to the surface of an implantable device by spin coating, dipping, or spraying. Additional methods known in the art can also be utilized for this purpose. Methods of spraying include traditional methods as well as microdeposition techniques with an inkjet type of dispenser. Additionally, a polymer can be deposited on an implantable device using photo-patterning to place the polymer on only specific portions of the device. This coating of the device provides a uniform layer around the device which allows for improved diffusion of various analytes through the device coating.

In certain embodiments of the invention, the compound is formulated for release from the polymer coating into the environment in which the medical device is placed. Preferably, the compound is released in a controlled manner over an extended time frame (e.g., months) using at least one of several well-known techniques involving polymer carriers or layers to control elution. Some of these techniques are described in U.S. Patent Application 2004/0243225A1, the entire disclosure of which is incorporated herein in its entirety.

Moreover, as described for example in U.S. Pat. No. 6,770,729, which is incorporated herein in its entirety, the reagents and reaction conditions of the polymer compositions can be manipulated so that the release of the compound from the polymer coating can be controlled. For example, the diffusion coefficient of the one or more polymer coatings can be modulated to control the release of the compound from the polymer coating. In a variation on this theme, the diffusion coefficient of the one or more polymer coatings can be controlled to modulate the ability of an analyte that is present in the environment in which the medical device is placed (e.g., an analyte that facilitates the breakdown or hydrolysis of some portion of the polymer) to access one or more components within the polymer composition (and for example, thereby modulate the release of the compound from the polymer coating). Yet another embodiment of the invention includes a device having a plurality of polymer coatings, each having a plurality of diffusion coefficients. In such embodiments of the invention, the release of the compound from the polymer coating can be modulated by the plurality of polymer coatings.

In yet another embodiment of the invention, the release of the compound from the polymer coating is controlled by modulating one or more of the properties of the polymer composition, such as the presence of one or more endogenous or exogenous compounds, or alternatively, the pH of the polymer composition. For example, certain polymer compositions can be designed to release a compound in response to a decrease in the pH of the polymer composition.

### Kits

The invention also provides a kit, comprising a compound of the invention, or a pharmaceutically acceptable salt thereof, at least one other therapeutic agent, packaging material, and instructions for administering the compound of the invention or the pharmaceutically acceptable salt thereof and the other therapeutic agent or agents to a mammal to treat or prevent a disease or condition characterized by aberrant complement activity. In one embodiment, the mammal is a human.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof.

### EXAMPLES

Having now described the present invention in detail, the same will be more clearly understood by reference to the following examples, which are included herewith for purposes of illustration only and are not intended to be limiting of the invention.

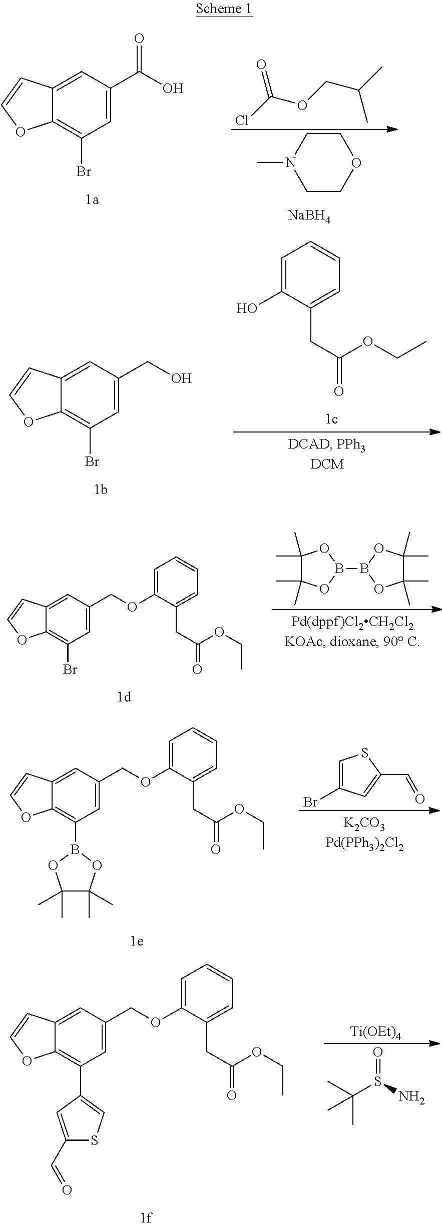

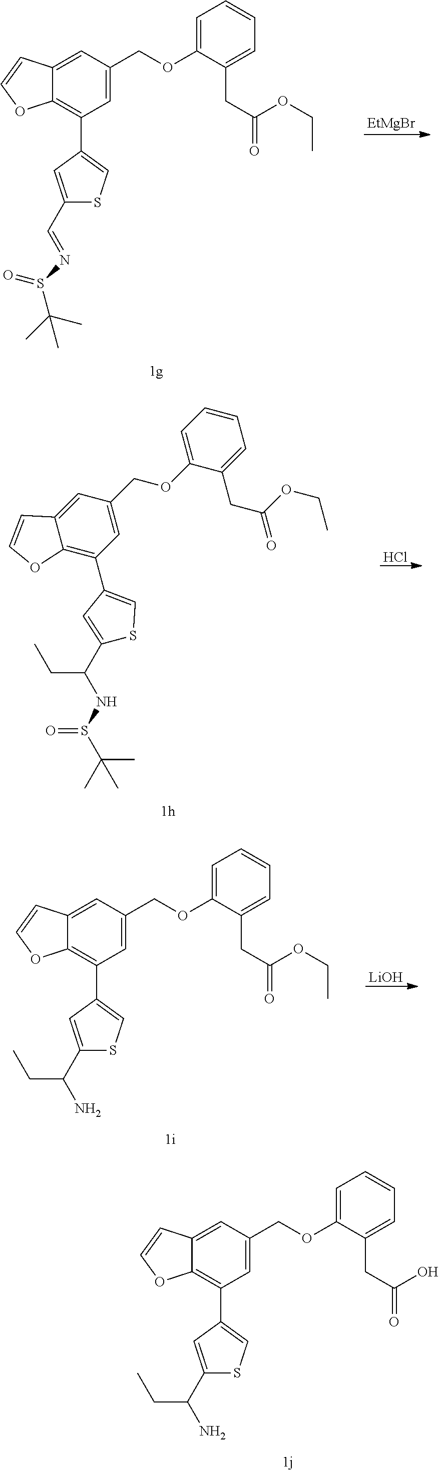

### Preparation of 2-(2-((7-(5-(1-aminopropyl)thiophen-3-yl)benzofuran-5-yl)methoxy)phenyl)acetic acid (1j)

### Step-1: Preparation of (7-bromobenzofuran-5-yl)methanol (1b)

To the stirred solution of 7-bromobenzofuran-5-carboxylic acid (1a) (10 g, 41.5 mmol; CAS #286836-25-7) and N-methylmorpholine (5.47 mL, 49.8 mmol) in THE (200 mL) at −5° C. was added isobutyl chloroformate (6.54 mL, 49.8 mmol). The reaction mixture was stirred for 15 min, filtered over a Celite pad and the precipitate was washed with THE (3×20 mL). The filtrate was cooled to 0° C. and added carefully (gas released rapidly) a solution of NaBH₄ (4.71 g, 124 mmol) in water (10 mL). The reaction mixture was stirred for 30 mins, diluted with water (20 mL), washed with ethyl acetate (3×). The organic layers were combined, dried, filtered and concentrated in vacuum to afford (7-bromobenzofuran-5-yl)methanol (1b) (9 g, 96% yield) as a white solid; ¹H NMR (300 MHz, DMSO-d₆) δ 8.09 (d, J=2.2 Hz, 1H), 7.59 (d, J=1.4 Hz, 1H), 7.49 (d, J=1.4 Hz, 1H), 7.07 (d, J=2.2 Hz, 1H), 5.34 (t, J=5.8 Hz, 1H), 4.57 (d, J=5.8 Hz, 2H).

### Step-2: Preparation of ethyl 2-(2-((7-bromobenzofuran-5-yl)methoxy)phenyl)acetate (1d)

To a solution of (7-bromobenzofuran-5-yl)methanol (1b) (9 g, 39.6 mmol), triphenylphosphine (11.44 g, 43.6 mmol) and ethyl 2-(2-hydroxyphenyl) acetate (1c) (7.86 g, 43.6 mmol; CAS #41873-65-8) in DCM (180 mL) at 0° C. was added dropwise a solution of bis(4-chlorobenzyl)azodicarboxylate (DCAD) (16.01 g, 43.6 mmol; CAS #: 916320-82-6) in DCM (40 mL). The resulting mixture was stirred at RT for 30 min and filtered to remove solid. The filtrate was concentrated in vacuum and residue obtained was purified by flash column chromatography [silica (80 g), eluting with EtOAc in hexane from 0-50%] to give ethyl 2-(2-((7-bromobenzofuran-5-yl)methoxy)phenyl)acetate (1d) (11.5 g, 75% yield) as a white solid; ¹H NMR (300 MHz, DMSO-d₆) δ 8.15 (d, J=2.2 Hz, 1H), 7.72 (s, 1H), 7.60 (s, 1H), 7.30-7.19 (m, 2H), 7.11 (d, J=2.2 Hz, 1H), 7.07 (d, J=8.1 Hz, 1H), 6.91 (td, J=7.4, 1.1 Hz, 1H), 5.18 (s, 2H), 4.01 (q, J=7.1 Hz, 2H), 3.62 (s, 2H), 1.07 (t, J=7.1 Hz, 3H).

### Step-3: Preparation of ethyl 2-(2-((7-(4,4,5,5-tetramethyl-1,3,2-dioxaborolan-2-yl)benzofuran-5-yl)methoxy)phenyl)acetate (1e)

To a degassed solution of ethyl 2-(2-((7-bromobenzofuran-5-yl)methoxy)phenyl)acetate (1d) (8.9 g, 22.87 mmol), bis(pinacolato)diboron (8.71 g, 34.3 mmol, CAS #: 73183-34-3) and potassium acetate (6.73 g, 68.6 mmol) in anhydrous dioxane (150 mL) was added Pd(dppf)Cl₂—CH₂Cl₂ (1.87 g, 2.29 mmol). The resulting mixture was degassed, filled with Ar and stirred at 90° C. overnight. The reaction mixture was then diluted with EtOAc (400 mL) and washed with water (100 mL). The aqueous layer was re-extracted with EtOAc (100 mL×2). The organic layers were combined washed with water (100 mL), brine (100 mL), dried and concentrated in vacuum. The residue obtained was purified by flash column chromatography [silica (80 g), eluting with EtOAc in hexane from 0-40%] to give ethyl 2-(2-((7-(4,4,5,5-tetramethyl-1,3,2-dioxaborolan-2-yl)benzofuran-5-yl)methoxy)phenyl)acetate (1e) (9 g, 90% yield) as an off white solid; ¹H NMR (300 MHz, DMSO-d₆) δ 8.05 (d, J=2.2 Hz, 1H), 7.84 (d, J=1.8 Hz, 1H), 7.65 (d, J=1.8 Hz, 1H), 7.34-7.17 (m, 2H), 7.10 (d, J=8.1 Hz, 1H), 6.98-6.87 (m, 2H), 5.17 (s, 2H), 4.00 (q, J=7.1 Hz, 2H), 3.59 (s, 2H), 1.34 (s, 12H), 1.05 (t, J=7.1 Hz, 3H).

### Step-4: Preparation of ethyl 2-(2-((7-(5-formylthiophen-3-yl)benzofuran-5-yl)methoxy)phenyl)acetate (1f)

To a degassed solution of ethyl 2-(2-((7-(4,4,5,5-tetramethyl-1,3,2-dioxaborolan-2-yl)benzofuran-5-yl)methoxy)phenyl)acetate (1e) (2 g, 4.58 mmol) in dioxane (24 mL) was added 4-bromothiophene-2-carbaldehyde (0.876 g, 5.01 mmol), bis(triphenylphosphine)Palladium(II)chloride [Pd(PPh₃)₂Cl₂](0.527 g, 0.751 mmol), a solution of K₂CO₃ (2.076 g, 15.02 mmol) in water (8 mL) and heated at 90° C. for 3 h on oil bath under a nitrogen atmosphere. The reaction mixture was cooled to room temperature diluted with EtOAc (100 mL) and water (15 mL). The organic layer was separated, and aqueous layer was extracted with ethyl acetate (100 mL). The combined organic layers were dried, filtered and evaporated to dryness. The residue obtained was purified by flash column chromatography [silica gel 12 g, eluting with DMA-80 in DCM from 0-50%] to give ethyl 2-(2-((7-(5-formylthiophen-3-yl)benzofuran-5-yl)methoxy)phenyl)acetate (1f) (2.022 g, 100% yield) as a yellow syrup. ¹H NMR (300 MHz, DMSO-d₆) δ 10.11-10.02 (m, 1H), 8.70 (s, 2H), 7.83 (d, J=1.6 Hz, 1H), 7.72 (d, J=1.6 Hz, 1H), 7.30-7.25 (m, 1H), 7.25-7.21 (m, 2H), 7.14-7.09 (m, 1H), 7.08 (d, J=2.2 Hz, 1H), 6.92 (td, J=7.4, 1.1 Hz, 1H), 5.24 (s, 2H), 3.98-3.92 (m, 2H), 3.65 (s, 2H), 1.00 (t, J=7.1 Hz, 3H).

### Step-5: Preparation of ethyl 2-(2-((7-(5-(((tert-butylsulfinyl)imino)methyl)thiophen-3-yl)benzofuran-5-yl)methoxy)phenyl)acetate (1g)

To a solution of ethyl 2-(2-((7-(5-formylthiophen-3-yl)benzofuran-5-yl)methoxy)phenyl)acetate (1f) (2.02 g, 9.32 mmol) and (R)-2-methylpropane-2-sulfinamide (2.258 g, 18.63 mmol) in tetrahydrofuran (30 mL) was added tetraethoxytitanium (3.91 mL, 18.63 mmol) and stirred overnight at RT. Reaction was quenched with brine (40 mL) and stirred for 20 minutes. The solid separated out was removed by filter and cake was washed with ethyl acetate (400 mL). The organic layer was separated washed with brine (2×50 mL), dried and concentrated in vacuum. The crude residue was purified by flash column chromatography [(silica gel, 40 g, eluting with ethyl acetate in hexanes (0 to 20%)] to afford ethyl 2-(2-((7-(5-(((tert-butylsulfinyl)imino)methyl)thiophen-3-yl)benzofuran-5-yl)methoxy)phenyl)acetate (1g) (1.596 g, 54% yield) as brown syrup. ¹H NMR (300 MHz, DMSO-d₆) δ 8.76 (s, 1H), 8.52 (d, J=1.3 Hz, 2H), 8.14 (d, J=2.2 Hz, 1H), 7.78 (d, J=1.6 Hz, 1H), 7.71 (d, J=1.6 Hz, 1H), 7.31-7.25 (m, 1H), 7.25-7.20 (m, 1H), 7.14-7.09 (m, 1H), 7.07 (d, J=2.2 Hz, 1H), 6.91 (td, J=7.4, 1.1 Hz, 1H), 5.24 (s, 2H), 3.96 (q, J=7.1 Hz, 2H), 3.65 (s, 2H), 1.19 (s, 9H), 1.00 (t, J=7.1 Hz, 3H).

### Step-6: Preparation of ethyl 2-(2-((7-(5-(1-(1,1-dimethylethylsulfinamido)propyl)thiophen-3-yl)benzofuran-5-yl)methoxy)phenyl)acetate (1h)

To a solution of ethyl 2-(2-((7-(5-(((tert-butylsulfinyl)imino)methyl)thiophen-3-yl)benzofuran-5-yl)methoxy)phenyl)acetate (1g) (0.3 g, 0.573 mmol) in THE (10 mL) at −78° C. was added carefully ethylmagnesium bromide (0.430 mL, 0.859 mmol) and stirred at −78° C. for 3 h. The reaction was carefully quenched with saturated ammonium chloride and the aqueous phase was extracted with ethyl acetate (3×20 mL). The organic layers were combined, washed with brine, dried, filtered and concentrated in vacuum. The residue obtained was purified by flash column chromatography [silica gel (12 g), eluting with EtOAc in hexane from 0-100%] to give ethyl 2-(2-((7-(5-(1-(1,1-dimethylethylsulfinamido)propyl)thiophen-3-yl)benzofuran-5-yl)methoxy)phenyl)acetate (1h) (0.318 g, 100% yield)¹H NMR (300 MHz, DMSO-d₆) δ 8.14-8.11 (m, 1H), 8.09-8.01 (m, 1H), 7.70 (d, J=1.8 Hz, 1H), 7.64 (d, J=1.5 Hz, 1H), 7.31-7.25 (m, 1H), 7.25-7.21 (m, 2H), 7.11 (d, J=8.4 Hz, 2H), 7.05 (d, J=2.2 Hz, 1H), 6.91 (t, J=7.4 Hz, 1H), 5.23 (s, 2H), 4.00-3.92 (m, 3H), 3.64 (s, 2H), 1.95-1.75 (m, 2H), 1.15 (s, 9H), 1.05-0.98 (m, 3H), 0.95-0.88 (m, 3H).

### Step-7: Preparation of ethyl 2-(2-((7-(5-(1-aminopropyl)thiophen-3-yl)benzofuran-5-yl)methoxy)phenyl)acetate (1i)

To a solution of ethyl 2-(2-((7-(5-(1-(1,1-dimethylethylsulfinamido)propyl)thiophen-3-yl)benzofuran-5-yl)methoxy)phenyl)acetate (1h) (318 mg, 0.574 mmol) in tetrahydrofuran (3 mL) was added aqueous 2N HCl (0.861 mL, 1.723 mmol) and stirred at RT for 4 h. The reaction mixture was concentrated to dryness and used in the next step without further purification. ¹H NMR (300 MHz, DMSO-d₆) δ 8.40 (s, 3H), 8.22 (s, 1H), 8.15 (d, J=2.2 Hz, 1H), 7.95 (s, 1H), 7.71 (d, J=9.4 Hz, 1H), 7.27 (s, 1H), 7.24 (d, J=6.0 Hz, 1H), 7.13 (s, 1H), 7.11-7.07 (m, 2H), 6.93 (s, 1H), 5.23 (s, 2H), 4.02-3.89 (m, 3H), 3.64 (s, 2H), 2.04-1.79 (m, 2H), 1.00 (t, J=7.1 Hz, 3H), 0.90 (t, J=7.5 Hz, 3H).

### Step-8: Preparation of 2-(2-((7-(5-(1-aminopropyl)thiophen-3-yl)benzofuran-5-yl)methoxy)phenyl)acetic acid (1j)

To a stirred solution of ethyl 2-(2-((7-(5-(1-aminopropyl)thiophen-3-yl)benzofuran-5-yl)methoxy)phenyl)acetate (1i) (255 mg, 0.510 mmol, from above step) in tetrahydrofuran (5 mL), methanol (1 mL) and water (1 mL) was added lithium hydroxide monohydrate (48.9 mg, 2.042 mmol) and stirred for 10 h at room temperature. The reaction was concentrated in vacuum diluted with 5 mL of water and acidified to pH 5 using 1M HCl. The solid separated was decanted and dissolved in DMSO and purified by reverse-phase column chromatography [C-18 column, 40 g, eluting with 0.1% aq HCl in water and acetonitrile from 0-100%] to afford 2-(2-((7-(5-(1-aminopropyl)thiophen-3-yl)benzofuran-5-yl)methoxy)phenyl)acetic acid (1j) (61 mg, 28.4% yield) hydrochloride salt as a white solid. ¹H NMR (300 MHz, DMSO-d₆) δ 12.14 (s, 1H), 8.53 (s, 3H, D₂O exchangeable), 8.22 (d, J=1.5 Hz, 1H), 8.15 (d, J=2.2 Hz, 1H), 7.96 (d, J=1.6 Hz, 1H), 7.78-7.68 (m, 2H), 7.29-7.19 (m, 2H), 7.09 (d, J=8.3 Hz, 1H), 7.06 (d, J=2.2 Hz, 1H), 6.91 (t, J=7.4 Hz, 1H), 5.26 (s, 2H), 4.65-4.54 (m, 1H), 3.61 (s, 2H), 2.14-1.85 (m, 2H), 0.91 (t, J=7.3 Hz, 3H); Analysis calculated for C₂₄H₂₃NO₄S·HCl·1.5H₂O: C, 59.44; H, 5.61; Cl, 7.31; N, 2.89; found: C, 59.56; H, 5.26; Cl, 7.52; N, 3.09.

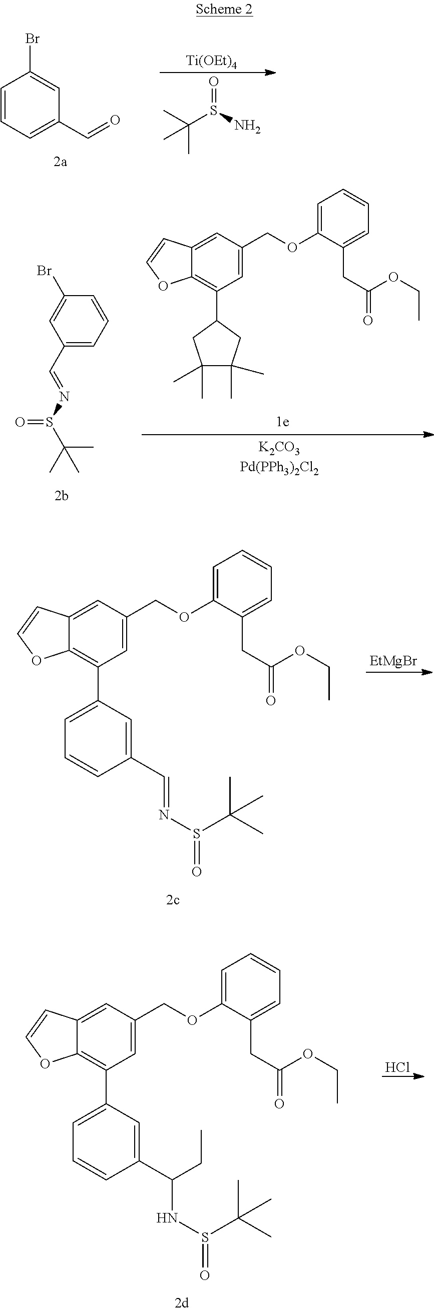

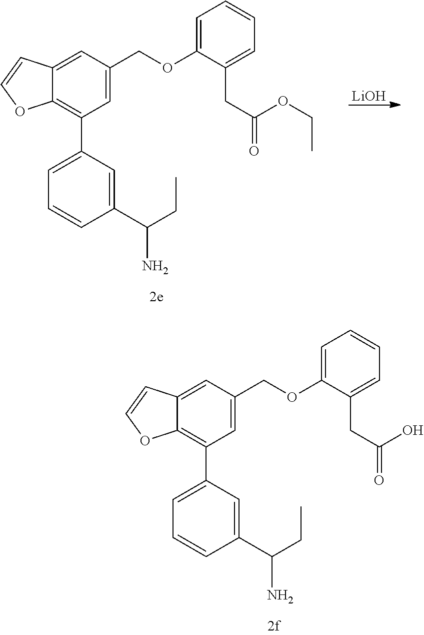

### Preparation of 2-(2-((7-(3-(1-aminopropyl)phenyl)benzofuran-5-yl)methoxy)phenyl)acetic acid (2f)

### Step-1: Preparation of N-(3-bromobenzylidene)-2-methylpropane-2-sulfinamide (2b)

Compound 2b was prepared according to procedure reported in Step-5 of scheme 1 from 3-bromobenzaldehyde (2a) (2 g, 10.81 mmol) in THE (30 mL) using (R)-2-methylpropane-2-sulfinamide (2.62 g, 21.62 mmol), tetraethoxytitanium (4.53 mL, 21.62 mmol) and stirring at room temperature for 18 h. This gave after workup and purification by flash column chromatography [(silica gel (40 g), eluting with ethyl acetate in hexanes (0 to 20%)]N-(3-bromobenzylidene)-2-methylpropane-2-sulfinamide (2b) (2.157 g, 69.2% yield) as brown syrup. ¹H NMR (300 MHz, DMSO-d₆) δ 8.55 (s, 1H), 8.11 (t, J=1.8 Hz, 1H), 7.94 (dt, J=7.7, 1.3 Hz, 1H), 7.76 (ddd, J=8.0, 2.1, 1.1 Hz, 1H), 7.49 (t, J=7.8 Hz, 1H), 1.17 (d, J=3.1 Hz, 9H).

### Step-2: Preparation of ethyl 2-(2-((7-(3-(((tert-butylsulfinyl)imino)methyl)phenyl)benzofuran-5-yl)methoxy)phenyl)acetate (2c)

Compound 2c was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (1.665 g, 3.82 mmol) in dioxane (24 mL) using N-(3-bromobenzylidene)-2-methylpropane-2-sulfinamide (2b) (1.1 g, 3.82 mmol), bis(triphenylphosphine)Palladium(II)chloride [Pd(PPh₃)₂Cl₂](0.402 g, 0.573 mmol), a solution of K₂CO₃ (1.583 g, 11.45 mmol) in water (8 mL) and heating at 90° C. for 3 h on oil bath. This gave after workup and purification by flash column chromatography [silica gel (12 g), eluting with DMA-80 in DCM from 0-50%]ethyl 2-(2-((7-(3-(((tert-butylsulfinyl)imino)methyl)phenyl)benzofuran-5-yl)methoxy)phenyl)acetate (2c) (1.64 g, 3.17 mmol, 83% yield) as a yellow syrup. ¹H NMR (300 MHz, DMSO-d₆) δ 8.68 (s, 1H), 8.44 (t, J=1.7 Hz, 1H), 8.14 (dd, J=5.5, 2.2 Hz, 2H), 8.06-8.01 (m, 1H), 7.71-7.66 (m, 1H), 7.28 (d, J=7.9 Hz, 1H), 7.25-7.21 (m, 2H), 7.14 (s, 1H), 7.09 (d, J=2.2 Hz, 1H), 6.91 (td, J=7.4, 1.1 Hz, 2H), 5.27 (d, J=1.9 Hz, 2H), 3.92 (q, J=7.1 Hz, 2H), 3.63 (d, J=2.6 Hz, 2H), 1.21 (s, 9H), 1.00-0.94 (m, 3H).

### Step-3: Preparation of ethyl 2-(2-((7-(3-(1-(1,1-dimethylethylsulfinamido)propyl)phenyl)benzofuran-5-yl)methoxy)phenyl)acetate (2d)

Compound 2d was prepared according to procedure reported in Step-6 of scheme 1 from ethyl 2-(2-((7-(3-(((tert-butylsulfinyl)imino)methyl)phenyl)benzofuran-5-yl)methoxy)phenyl)acetate (2c) (0.3 g, 0.580 mmol) in THE (10 mL) at −78° C. using ethylmagnesium bromide (0.435 mL, 0.869 mmol) and stirring at −78° C. for 3 h. This gave after workup and purification by flash column chromatography [silica gel (12 g), eluting with EtOAc in hexane from 0-100%]ethyl 2-(2-((7-(3-(1-(1,1-dimethylethylsulfinamido)propyl)phenyl)benzofuran-5-yl)methoxy)phenyl)acetate (2d) (279 mg, 88% yield); MS (ES+) 548.2 (M+1).

### Step-4: Preparation of ethyl 2-(2-((7-(3-(1-aminopropyl)phenyl)benzofuran-5-yl)methoxy)phenyl)acetate (2e)

To a solution of ethyl 2-(2-((7-(3-(1-(1,1-dimethylethylsulfinamido)propyl)phenyl)benzofuran-5-yl)methoxy)phenyl)acetate (2d) (279 mg, 0.509 mmol) in tetrahydrofuran (3 mL) was added aqueous 2N HCl (0.764 mL, 1.528 mmol) and stirred at RT for 4 h. The reaction mixture was concentrated to dryness and used in the next step without further purification; MS (ES+) 444.2 (M+1).

### Step-5: Preparation of 2-(2-((7-(3-(1-aminopropyl)phenyl)benzofuran-5-yl)methoxy)phenyl)acetic acid (2f)

Compound 2f was prepared according to procedure reported in Step-8 of scheme 1 from ethyl 2-(2-((7-(3-(1-aminopropyl)phenyl)benzofuran-5-yl)methoxy)phenyl)acetate (2e) (226 mg, 0.510 mmol) in THE (5 mL), methanol (1 mL) and water (1 mL) using lithium hydroxide monohydrate (86 mg, 2.038 mmol) and stirring for 10 h at room temperature. This gave after workup and purification by reverse-phase column chromatography [C-18 column (40 g), eluting with 0.1% aq HCl in water and acetonitrile from 0-100%]2-(2-((7-(3-(1-aminopropyl)phenyl)benzofuran-5-yl)methoxy)phenyl)acetic acid (2f) (46 mg, 21.73% yield) hydrochloride salt as a white solid. ¹H NMR (300 MHz, DMSO-d₆) δ 12.25 (s, 1H, D₂O exchangeable), 8.45 (s, 3H, D₂O exchangeable), 8.12 (d, J=2.2 Hz, 1H), 8.01-7.98 (m, 1H), 7.96-7.92 (m, 1H), 7.81-7.74 (m, 1H), 7.68-7.64 (m, 1H), 7.61 (d, J=7.6 Hz, 1H), 7.58-7.50 (m, 1H), 7.31-7.19 (m, 2H), 7.10 (d, J=8.0 Hz, 1H), 7.07 (d, J=2.2 Hz, 1H), 6.97-6.86 (m, 1H), 5.28 (s, 2H), 4.31-4.20 (m, 1H), 3.60 (s, 2H), 2.11-1.82 (m, 2H), 0.84 (t, J=7.4 Hz, 3H). MS (ES+): 416.1 (M+1); Analysis calculated for C₂₆H₂₅NO₄·HCl·1.25H₂O: C, 65.82; H, 6.05; Cl, 7.47; N, 2.95; found: C, 65.89; H, 5.83; Cl, 7.61; N, 3.17.

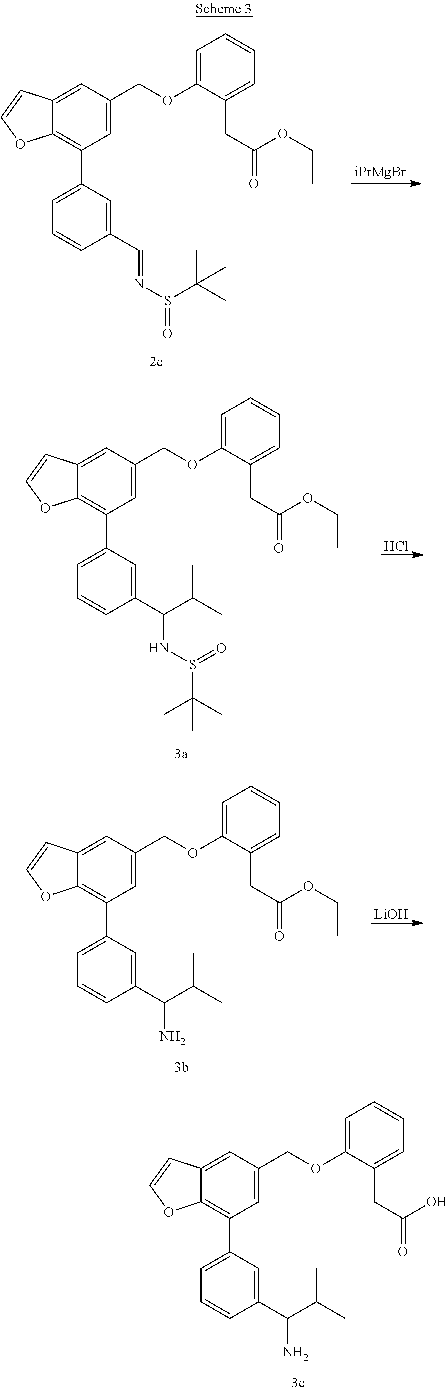

### Preparation of 2-(2-((7-(3-(1-amino-2-methylpropyl)phenyl)benzofuran-5-yl)methoxy)phenyl)acetic acid (3c)

### Step-1: Preparation of ethyl 2-(2-((7-(3-(1-(1,1-dimethylethylsulfinamido)-2-methylpropyl)phenyl)benzofuran-5-yl)methoxy)phenyl)acetate (3a)

Compound 3a was prepared according to procedure reported in Step-6 of scheme 1 from ethyl 2-(2-((7-(3-(((tert-butylsulfinyl)imino)methyl)phenyl)benzofuran-5-yl)methoxy)phenyl)acetate (2c) (0.400 g, 0.773 mmol) in THE (10 mL) at −78° C. using isopropylmagnesium bromide (2.318 mL, 4.64 mmol) and stirring at −78° C. for 3 h. This gave after workup and purification by flash column chromatography [silica gel (12 g), eluting with EtOAc in hexane from 0-100%]ethyl 2-(2-((7-(3-(1-(1,1-dimethylethylsulfinamido)-2-methylpropyl)phenyl)benzofuran-5-yl)methoxy)phenyl)acetate (3a) (360 mg, 83% yield); MS (ES+): 562.3 (M+1).

### Step-2: Preparation of ethyl 2-(2-((7-(3-(1-amino-2-methylpropyl)phenyl)benzofuran-5-yl)methoxy)phenyl)acetate (3b)

To a solution of ethyl 2-(2-((7-(3-(1-(1,1-dimethylethylsulfinamido)-2-methylpropyl)phenyl)benzofuran-5-yl)methoxy)phenyl)acetate (3a) (360 mg, 0.641 mmol) in tetrahydrofuran (3 mL) was added aqueous 2N HCl (0.764 mL, 1.528 mmol) and stirred at RT for 4 h. The reaction mixture was concentrated to dryness to afford ethyl 2-(2-((7-(3-(1-amino-2-methylpropyl)phenyl)benzofuran-5-yl)methoxy)phenyl)acetate (3b) which was used in the next step without further purification; MS (ES+): 458.2 (M+1).

### Step-3: Preparation of 2-(2-((7-(3-(1-amino-2-methylpropyl)phenyl)benzofuran-5-yl)methoxy)phenyl)acetic acid (3c)

Compound 3c was prepared according to procedure reported in Step-8 of scheme 1 from ethyl 2-(2-((7-(3-(1-amino-2-methylpropyl)phenyl)benzofuran-5-yl)methoxy)phenyl)acetate (3b) (297 mg, 0.649 mmol) in THE (5 mL), methanol (1 mL) and water (1 mL) using lithium hydroxide monohydrate (62.2 mg, 2.60 mmol) and stirring for 10 h at room temperature. This gave after workup and purification by reverse-phase column chromatography [C-18 column (40 g), eluting with 0.1% aq HCl in water and acetonitrile from 0-100%]2-(2-((7-(3-(1-amino-2-methylpropyl)phenyl)benzofuran-5-yl)methoxy)phenyl)acetic acid (3c) (16 mg, 6% yield) hydrochloride salt as a white solid. ¹H NMR (300 MHz, DMSO-d₆) δ 12.23 (s, 1H, D₂O exchangeable), 8.49-8.43 (m, 3H, D₂O exchangeable), 8.12 (d, J=2.2 Hz, 1H), 8.00-7.89 (m, 2H), 7.79-7.76 (m, 1H), 7.68-7.58 (m, 2H), 7.50 (d, J=7.7 Hz, 1H), 7.23 (m, J=7.5 Hz, 2H), 7.11 (d, J=8.0 Hz, 1H), 7.07 (d, J=2.2 Hz, 1H), 6.91 (t, J=7.4 Hz, 1H), 5.28 (s, 2H), 4.17-4.00 (m, 1H), 3.60 (s, 2H), 2.31-2.15 (m, 1H), 1.07 (d, J=6.6 Hz, 3H), 0.80 (d, J=6.7 Hz, 3H); MS (ES+): 430.2 (M+1).

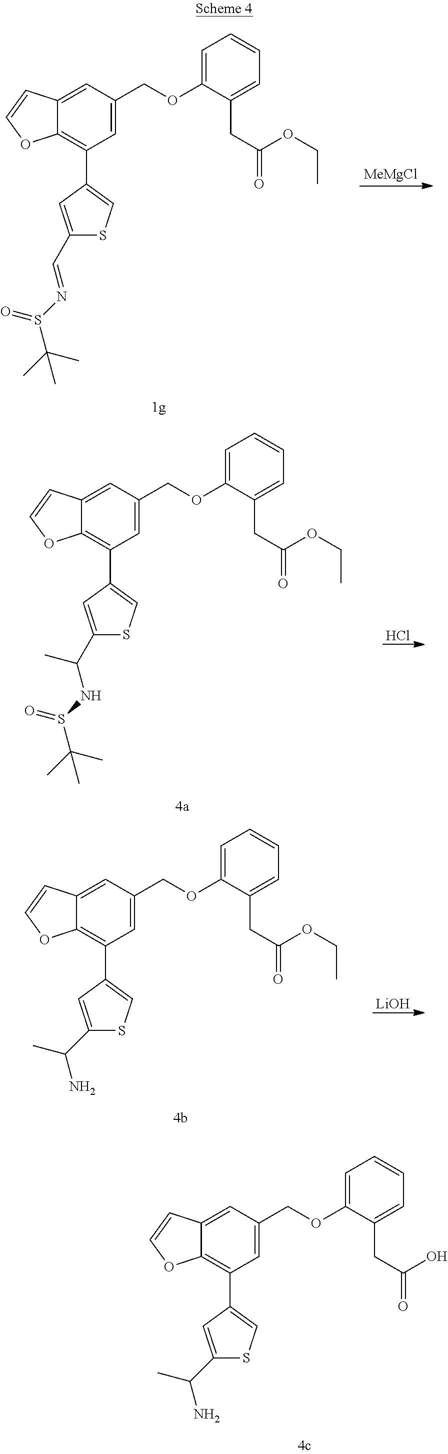

### Preparation of 2-(2-((7-(5-(1-aminoethyl)thiophen-3-yl)benzofuran-5-yl)methoxy)phenyl)acetic acid (4c)

### Step-1: Preparation of ethyl 2-(2-((7-(5-(1-(1,1-dimethylethylsulfinamido)ethyl)thiophen-3-yl)benzofuran-5-yl)methoxy)phenyl)acetate (4a)

Compound 4a was prepared according to procedure reported in Step-6 of scheme 1 from ethyl 2-(2-((7-(5-(((tert-butylsulfinyl)imino)methyl)thiophen-3-yl)benzofuran-5-yl)methoxy)phenyl)acetate (1g) (0.4 g, 0.773 mmol) in THE (10 mL) at −78° C. using methyl magnesium chloride (1.528 mL, 4.58 mmol) and stirring at −78° C. for 3 h. This gave after workup and purification by flash column chromatography [silica gel (12 g), eluting with EtOAc in hexane from 0-100%]ethyl 2-(2-((7-(5-(1-(1,1-dimethylethylsulfinamido)ethyl)thiophen-3-yl)benzofuran-5-yl)methoxy)phenyl)acetate (4a) (301 mg, 0.530 mmol, 69.4% yield). MS (ES+): 540.2 (M+1)

### Step-2: Preparation of ethyl 2-(2-((7-(5-(1-aminoethyl)thiophen-3-yl)benzofuran-5-yl)methoxy)phenyl)acetate (4b)

To a solution of ethyl 2-(2-((7-(5-(1-(1,1-dimethylethylsulfinamido)ethyl)thiophen-3-yl)benzofuran-5-yl)methoxy)phenyl)acetate (4a) (301 mg, 0.558 mmol) in tetrahydrofuran (3 mL) was added aqueous 2N HCl (0.837 mL, 1.673 mmol) and stirred at RT for 4 h. The reaction mixture was concentrated to dryness to afford ethyl 2-(2-((7-(5-(1-aminoethyl)thiophen-3-yl)benzofuran-5-yl)methoxy)phenyl)acetate (4b) which was used in the next step without further purification. MS (ES+): 437.0 (M+1);

### Step-3: Preparation of 2-(2-((7-(5-(1-aminoethyl)thiophen-3-yl)benzofuran-5-yl)methoxy)phenyl)acetic acid (4c)

Compound 4c was prepared according to procedure reported in Step-8 of scheme 1 from ethyl 2-(2-((7-(5-(1-aminoethyl)thiophen-3-yl)benzofuran-5-yl)methoxy)phenyl)acetate (4b) (243 mg, 0.558 mmol) in THE (5 mL), methanol (1 mL) and water (1 mL) using lithium hydroxide monohydrate (53.4 mg, 2.232 mmol) and stirring for 10 h at room temperature. This gave after workup and purification by reverse-phase column chromatography [C-18 column (40 g), eluting with 0.1% aqueous HCl in water and acetonitrile from 0-100%]2-(2-((7-(5-(1-aminoethyl)thiophen-3-yl)benzofuran-5-yl)methoxy)phenyl)acetic acid (4c) (28 mg, 12% yield) hydrochloride salt as a white solid. ¹H NMR (300 MHz, DMSO-d₆) δ 8.50 (s, 3H), 8.22-8.17 (m, 1H), 8.17-8.13 (m, 1H), 7.95 (d, J=11.0 Hz, 1H), 7.75 (d, J=10.5 Hz, 1H), 7.69 (d, J=5.4 Hz, 1H), 7.31-7.13 (m, 2H), 7.11-7.07 (m, 2H), 6.96-6.88 (m, 1H), 5.27-5.15 (m, 2H), 4.82 (s, 1H), 3.73 (s, 2H), 1.66 (dd, J=6.9, 2.3 Hz, 3H). MS (ES−): 405.1 (M−1); Analysis calculated for C₂₃H₂₁NO₄S·HCl·0.5H₂O: C, 60.99; H, 5.12; Cl, 7.83; N, 3.09; found: C, 61.10; H, 5.45; Cl, 7.47; N, 2.98.

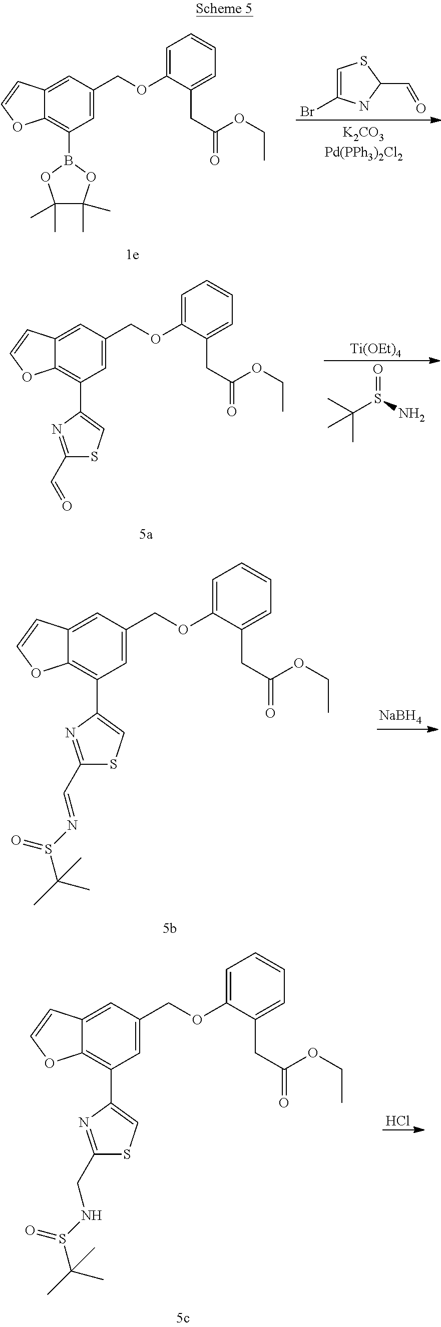

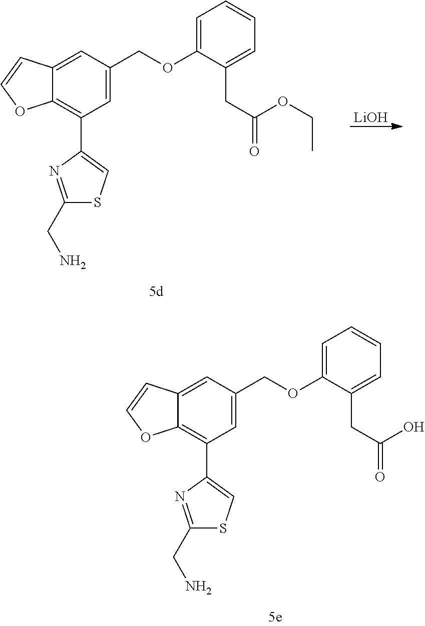

### Preparation of 2-(2-((7-(2-(aminomethyl)thiazol-4-yl)benzofuran-5-yl)methoxy)phenyl)acetic acid (5e)

### Step-1: Preparation of ethyl 2-(2-((7-(2-formylthiazol-4-yl)benzofuran-5-yl)methoxy)phenyl)acetate (5a)

Compound 5a was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (1 g, 2.292 mmol) in dioxane (12 mL) using 2-bromothiazole-4-carbaldehyde (0.440 g, 2.292 mmol), bis(triphenylphosphine)Palladium(II)chloride [Pd(PPh₃)₂Cl₂](0.241 g, 0.344 mmol), a solution of K₂CO₃ (0.950 g, 6.88 mmol) in water (4 mL) and heating at 90° C. for 3 h on oil bath. This gave after workup and purification by flash column chromatography [silica gel (12 g), eluting with DMA-80 in DCM from 0-50%]ethyl 2-(2-((7-(2-formylthiazol-4-yl)benzofuran-5-yl)methoxy)phenyl)acetate (5a) (664 mg, 1.575 mmol, 68.7% yield) as a yellow syrup; ¹H NMR (300 MHz, DMSO-d₆) δ 10.08 (d, J=1.3 Hz, 1H), 8.84 (d, J=1.3 Hz, 1H), 8.22 (d, J=2.2 Hz, 1H), 8.18 (d, J=1.7 Hz, 1H), 7.80 (t, J=2.5 Hz, 1H), 7.57-7.47 (m, 1H), 7.40 (dd, J=14.1, 7.0 Hz, 1H), 7.23 (dt, J=7.6, 2.0 Hz, 1H), 7.14-7.07 (m, 1H), 6.96-6.88 (m, 1H), 5.28 (d, J=2.4 Hz, 2H), 4.03-3.93 (m, 2H), 3.63 (s, 2H), 1.00 (t, J=7.1 Hz, 3H); MS (ES+): 422.0 (M+1).

### Step-2: Preparation of ethyl 2-(2-((7-(2-(((tert-butylsulfinyl)imino)methyl)thiazol-4-yl)benzofuran-5-yl)methoxy)phenyl)acetate (5b)

Compound 5b was prepared according to procedure reported in Step-5 of scheme 1 from 3 ethyl 2-(2-((7-(2-formylthiazol-4-yl)benzofuran-5-yl)methoxy)phenyl)acetate (5a) (950 mg, 2.254 mmol) in THE (15 mL) using (R)-2-methylpropane-2-sulfinamide (546 mg, 4.51 mmol), tetraethoxytitanium (0.945 mL, 4.51 mmol) and stirring at room temperature for 18 h.

This gave after workup and purification by flash column chromatography [(silica gel (40 g), eluting with ethyl acetate in hexanes (0 to 20%)]ethyl 2-(2-((7-(2-(((tert-butylsulfinyl)imino)methyl)thiazol-4-yl)benzofuran-5-yl)methoxy)phenyl)acetate (5b) (1.142 g, 2.177 mmol, 97% yield) as brown syrup; ¹H NMR (300 MHz, DMSO-d₆) δ 8.71 (d, J=1.0 Hz, 1H), 8.68 (d, J=1.0 Hz, 1H), 8.21 (d, J=2.2 Hz, 1H), 8.17 (d, J=1.7 Hz, 1H), 7.79 (d, J=1.7 Hz, 1H), 7.28 (d, J=7.3 Hz, 1H), 7.23 (d, J=8.9 Hz, 2H), 7.13 (d, J=2.2 Hz, 1H), 6.92 (td, J=7.4, 1.2 Hz, 1H), 5.32 (s, 2H), 4.03-3.95 (m, 2H), 3.63 (s, 2H), 1.08 (s, 9H), 1.01 (t, J=7.1 Hz, 3H); MS (ES+): 525.1 (M+1).

### Step-3: Preparation of ethyl 2-(2-((7-(2-((1,1-dimethylethylsulfinamido)methyl)thiazol-4-yl)benzofuran-5-yl)methoxy)phenyl)acetate (5c)

To a solution of ethyl 2-(2-((7-(2-(((tert-butylsulfinyl)imino)methyl)thiazol-4-yl)benzofuran-5-yl)methoxy)phenyl)acetate (5b) (862 mg, 1.643 mmol) in THE (10 mL) and water (1 mL) was added sodium borohydride (186 mg, 4.93 mmol) portion wise below −56° C. over a period of 2 mins. The reaction was then stirred at the same temperature for 30 min and allowed to warm to −12° C. over a period of 30 min. Reaction was quenched by adding acetone (1 mL), stirred for 10 mins diluted with water (10 mL) and concentrated to remove acetone and tetrahydrofuran. The residue was dissolved in ethyl acetate (100 mL), washed with water (2×20 mL), brine (20 mL), dried and concentrated to get a clear oil. The crude residue was purified by flash column chromatography [(silica gel (25 g), eluting with ethyl acetate in hexanes (0 to 100%)] to afford ethyl 2-(2-((7-(2-((1,1-dimethylethylsulfinamido)methyl)thiazol-4-yl)benzofuran-5-yl)methoxy)phenyl)acetate (5c) (236 mg, 0.448 mmol, 27.3% yield) as a white solid; ¹H NMR (300 MHz, DMSO-d₆) δ 8.28 (s, 1H), 8.18 (d, J=2.2 Hz, 1H), 8.10 (d, J=1.7 Hz, 1H), 7.72 (d, J=1.7 Hz, 1H), 7.30-7.25 (m, 1H), 7.25-7.21 (m, 1H), 7.13 (d, J=8.1 Hz, 1H), 7.09 (d, J=2.2 Hz, 1H), 6.92 (td, J=7.4, 1.1 Hz, 1H), 6.42 (t, J=6.2 Hz, 1H), 5.25 (s, 2H), 4.57 (t, J=6.6 Hz, 2H), 4.02-3.95 (m, 2H), 3.62 (s, 2H), 1.23 (s, 9H), 1.02 (t, J=7.1 Hz, 3H); MS (ES+): 527.1 (M+1).

### Step-4: Preparation of ethyl 2-(2-((7-(2-(aminomethyl)thiazol-4-yl)benzofuran-5-yl)methoxy)phenyl)acetate (5d)

To a solution of ethyl 2-(2-((7-(2-((1,1-dimethylethylsulfinamido)methyl)thiazol-4-yl)benzofuran-5-yl)methoxy)phenyl)acetate (5c) (234 mg, 0.444 mmol) in tetrahydrofuran (3 mL) was added aqueous 2N HCl (0.666 mL, 1.333 mmol) and stirred at RT for 4 h. The reaction mixture was concentrated to dryness to afford ethyl 2-(2-((7-(2-(aminomethyl)thiazol-4-yl)benzofuran-5-yl)methoxy)phenyl)acetate (5d) which was used as such in the next step without further purification; (ES+): 423.1 (M+1).

### Step-5: Preparation of 2-(2-((7-(2-(aminomethyl)thiazol-4-yl)benzofuran-5-yl)methoxy)phenyl)acetic acid (5e)

Compound 5e was prepared according to procedure reported in Step-8 of scheme 1 from ethyl 2-(2-((7-(2-(aminomethyl)thiazol-4-yl)benzofuran-5-yl)methoxy)phenyl)acetate (5d) (188 mg, 0.445 mmol) in THE (5 mL), methanol (1 mL) and water (1 mL) using lithium hydroxide monohydrate (42.6 mg, 1.780 mmol) and stirring for 10 h at room temperature. This gave after workup and purification by reverse-phase column chromatography [C-18 column (40 g), eluting with 0.1% aq HCl in water and acetonitrile from 0-100%]2-(2-((7-(2-(aminomethyl)thiazol-4-yl)benzofuran-5-yl)methoxy)phenyl)acetic acid (5e) (83 mg, 47% yield) hydrochloride salt as a white solid. ¹H NMR (300 MHz, DMSO-d₆) δ 8.74-8.63 (m, 3H, D₂O exchangeable), 8.41 (s, 1H), 8.21 (d, J=1.7 Hz, 1H), 8.20 (d, J=2.2 Hz, 1H), 7.79 (d, J=1.7 Hz, 1H), 7.27-7.19 (m, 2H), 7.14-7.05 (m, 2H), 6.95-6.88 (m, 1H), 5.27 (s, 2H), 4.60-4.51 (m, 2H), 3.60 (s, 2H); MS (ES+): 395.1 (M+1); Analysis calculated for C₂₁H₁₈N₂O₄S·1.3HCl·1.5H₂O: C, 53.79; H, 4.79; Cl, 9.83; N, 5.97; Found: C, 53.83; H, 4.69; Cl, 9.93; N, 5.88.

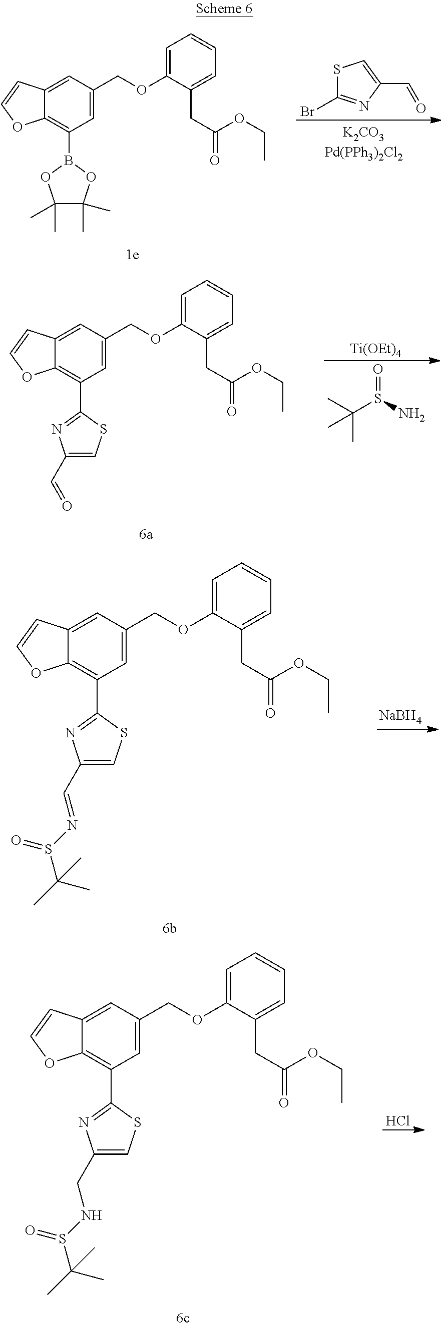

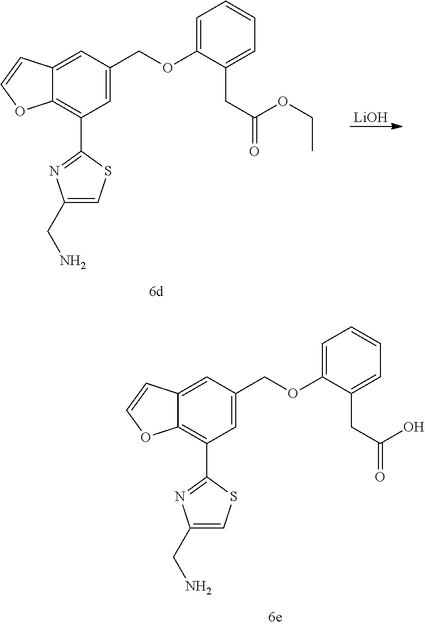

### Preparation of 2-(2-((7-(4-(aminomethyl)thiazol-2-yl)benzofuran-5-yl)methoxy)phenyl)acetic acid (6e)

### Step-1: Preparation of ethyl 2-(2-((7-(4-formylthiazol-2-yl)benzofuran-5-yl)methoxy)phenyl)acetate (6a)

Compound 6a was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (1 g, 2.292 mmol) in dioxane (12 mL) using 2-bromothiazole-4-carbaldehyde (0.440 g, 2.292 mmol), bis(triphenylphosphine)Palladium(II)chloride [Pd(PPh₃)₂Cl₂](0.241 g, 0.344 mmol), a solution of K₂CO₃ (0.950 g, 6.88 mmol) in water (4 mL) and heating at 90° C. for 3 h on oil bath. This gave after workup and purification by flash column chromatography [silica gel (12 g), eluting with DMA-80 in DCM from 0-50%]ethyl 2-(2-((7-(4-formylthiazol-2-yl)benzofuran-5-yl)methoxy)phenyl)acetate (6a) (664 mg, 68.7% yield) as a yellow syrup; ¹H NMR (300 MHz, DMSO-d₆) δ 10.06 (s, 1H), 8.94 (s, 1H), 8.31 (d, J=2.2 Hz, 1H), 8.24 (d, J=1.6 Hz, 1H), 7.92 (d, J=1.7 Hz, 1H), 7.27 (s, 1H), 7.24 (d, J=7.3 Hz, 1H), 7.19 (d, J=2.2 Hz, 1H), 7.14 (d, J=8.1 Hz, 1H), 6.96-6.89 (m, 1H), 5.30 (s, 2H), 4.00 (q, J=7.1 Hz, 2H), 3.63 (s, 2H), 1.00 (t, J=7.1 Hz, 3H); MS (ES+): 422.1 (M+1).

### Step-2: Preparation of ethyl 2-(2-((7-(4-(((tert-butylsulfinyl)imino)methyl)thiazol-2-yl)benzofuran-5-yl)methoxy)phenyl)acetate (6b)

Compound 6b was prepared according to procedure reported in Step-5 of scheme 1 from ethyl 2-(2-((7-(4-formylthiazol-2-yl)benzofuran-5-yl)methoxy)phenyl)acetate (6a) (664 mg, 1.575 mmol) in THE (15 mL) using (R)-2-methylpropane-2-sulfinamide (382 mg, 3.15 mmol), tetraethoxytitanium (0.661 mL, 3.15 mmol) and stirring at room temperature for 18 h. This gave after workup and purification by flash column chromatography [(silica gel (40 g), eluting with ethyl acetate in hexanes (0 to 20%)]ethyl 2-(2-((7-(4-(((tert-butylsulfinyl)imino)methyl)thiazol-2-yl)benzofuran-5-yl)methoxy)phenyl)acetate (6b) (528 mg, 63.9% yield) as brown syrup; ¹H NMR (300 MHz, DMSO-d₆) δ 8.80 (s, 1H), 8.66 (s, 1H), 8.31 (d, J=2.2 Hz, 1H), 8.24 (d, J=1.6 Hz, 1H), 7.91 (d, J=1.6 Hz, 1H), 7.29 (d, J=7.1 Hz, 1H), 7.23 (d, J=8.0 Hz, 1H), 7.19 (d, J=2.2 Hz, 1H), 7.14 (d, J=8.2 Hz, 1H), 6.92 (t, J=7.3 Hz, 1H), 5.30 (s, 2H), 4.02-3.95 (m, 2H), 3.62 (s, 2H), 1.22 (s, 9H), 0.98 (t, J=7.1 Hz, 3H); MS (ES+): 525.1 (M+1).

### Step-3: Preparation of ethyl 2-(2-((7-(4-((1,1-dimethylethylsulfinamido)methyl)thiazol-2-yl)benzofuran-5-yl)methoxy)phenyl)acetate (6c)

Compound 6c was prepared according to procedure reported in Step-3 of scheme 5 from ethyl 2-(2-((7-(4-(((tert-butylsulfinyl)imino)methyl)thiazol-2-yl)benzofuran-5-yl)methoxy)phenyl)acetate (6b) (528 mg, 1.006 mmol) in THE (10 mL) and water (1 mL) using sodium borohydride (114 mg, 3.02 mmol). This gave after workup and purification by flash column chromatography [(silica gel (25 g), eluting with ethyl acetate in hexanes (0 to 100%)]ethyl 2-(2-((7-(4-((1,1-dimethylethylsulfinamido)methyl)thiazol-2-yl)benzofuran-5-yl)methoxy)phenyl)acetate (6c) (484 mg, 91% yield) as a white solid; ¹H NMR (300 MHz, DMSO-d₆) δ 8.26 (d, J=2.2 Hz, 1H), 8.16 (d, J=1.7 Hz, 1H), 7.84 (d, J=1.7 Hz, 1H), 7.71 (s, 1H), 7.28 (dd, J=7.9, 1.8 Hz, 1H), 7.25-7.21 (m, 1H), 7.15 (d, J=2.2 Hz, 2H), 6.92 (td, J=7.4, 1.1 Hz, 1H), 5.94 (t, J=6.0 Hz, 1H), 5.27 (s, 2H), 4.40 (dd, J=6.2, 3.5 Hz, 2H), 4.01-3.94 (m, 2H), 3.62 (s, 2H), 1.19 (s, 9H), 1.01 (t, J=7.1 Hz, 3H); MS (ES+): 527.2 (M+1).

### Step-4: Preparation of ethyl 2-(2-((7-(4-(aminomethyl)thiazol-2-yl)benzofuran-5-yl)methoxy)phenyl)acetate (6d)

To a solution of ethyl 2-(2-((7-(4-((1,1-dimethylethylsulfinamido)methyl)thiazol-2-yl)benzofuran-5-yl)methoxy)phenyl)acetate (6c) (484 mg, 0.919 mmol) in tetrahydrofuran (3 mL) was added aqueous 2N HCl (1.378 mL, 2.76 mmol) and stirred at RT for 4 h. The reaction mixture was concentrated to dryness to afford ethyl 2-(2-((7-(4-(aminomethyl)thiazol-2-yl)benzofuran-5-yl)methoxy)phenyl)acetate (6d) which was used as such in the next step without further purification; (ES+): 423.1 (M+1)

### Step-5: Preparation of 2-(2-((7-(4-(aminomethyl)thiazol-2-yl)benzofuran-5-yl)methoxy)phenyl)acetic acid (6e)

Compound 6e was prepared according to procedure reported in Step-8 of scheme 1 from ethyl 2-(2-((7-(4-(aminomethyl)thiazol-2-yl)benzofuran-5-yl)methoxy)phenyl)acetate (6d) (388 mg, 0.918 mmol) in THE (5 mL), methanol (1 mL) and water (1 mL) using lithium hydroxide monohydrate (154 mg, 3.67 mmol) and stirring for 10 h at room temperature. This gave after workup and purification by reverse-phase column chromatography [C-18 column (40 g), eluting with 0.1% aq HCl in water and acetonitrile from 0-100%]2-(2-((7-(4-(aminomethyl)thiazol-2-yl)benzofuran-5-yl)methoxy)phenyl)acetic acid (6e) (41 mg, 11.32% yield) white hydrochloride salt. ¹H NMR (300 MHz, DMSO-d₆) δ 8.50 (s, 3H, D₂O exchangeable), 8.28 (d, J=2.2 Hz, 1H), 8.24 (d, J=1.7 Hz, 1H), 7.98 (s, 1H), 7.92 (d, J=1.7 Hz, 1H), 7.26-7.21 (m, 2H), 7.16 (d, J=2.2 Hz, 1H), 7.10 (d, J=8.1 Hz, 1H), 6.92 (t, J=7.1 Hz, 1H), 5.30 (s, 2H), 4.37-4.22 (m, 2H), 3.61 (s, 2H). MS (ES+): 395.1 (M+1); Analysis calculated for C₂₁H₁₈N₂O₄S·1.25HCl·2H₂O: C, 52.98; H, 4.92; Cl, 9.31; N, 5.88; Found: C, 52.80; H, 4.98; Cl, 9.79; N, 5.93.

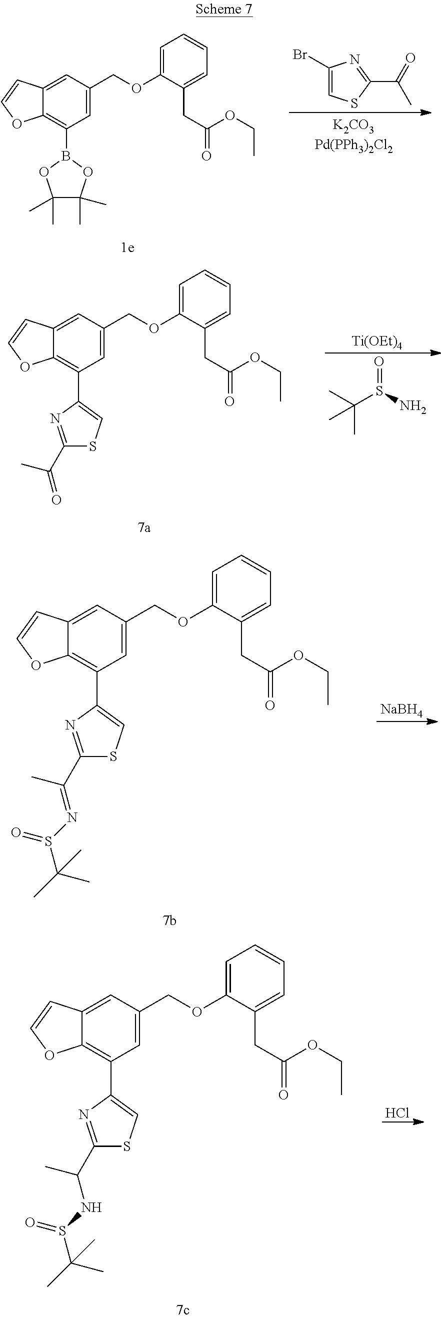

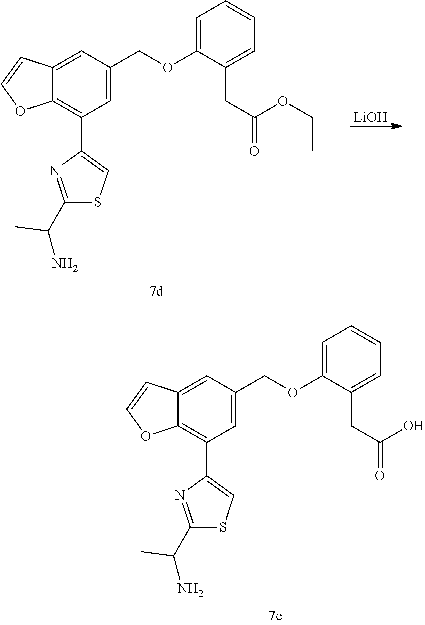

### Preparation of 2-(2-((7-(2-(1-aminoethyl)thiazol-4-yl)benzofuran-5-yl)methoxy)phenyl)acetic acid (7e)

### Step-1: Preparation of ethyl 2-(2-((7-(2-acetylthiazol-4-yl)benzofuran-5-yl)methoxy)phenyl)acetate (7a)

Compound 7a was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (1 g, 2.292 mmol) in dioxane (12 mL) using 1-(4-bromothiazol-2-yl)ethanone (0.472 g, 2.292 mmol), bis(triphenylphosphine)Palladium(II)chloride [Pd(PPh₃)₂Cl₂](0.241 g, 0.344 mmol), a solution of K₂CO₃ (0.950 g, 6.88 mmol) in water (4 mL) and heating at 90° C. for 3 h on oil bath. This gave after workup and purification by flash column chromatography [silica gel (12 g), eluting with DMA-80 in DCM from 0-50%]ethyl 2-(2-((7-(2-acetylthiazol-4-yl)benzofuran-5-yl)methoxy)phenyl)acetate (7a) (950 mg, 98% yield) as a yellow syrup; ¹H NMR (300 MHz, DMSO-d₆) δ 8.74 (s, 1H), 8.20 (t, J=2.0 Hz, 1H), 7.78 (d, J=1.7 Hz, 1H), 7.27 (d, J=1.8 Hz, 1H), 7.26-7.19 (m, 2H), 7.16 (s, 1H), 7.12 (t, J=2.2 Hz, 1H), 6.92 (td, J=7.4, 1.1 Hz, 1H), 5.28 (s, 2H), 3.97 (q, J=7.1 Hz, 2H), 3.65 (s, 2H), 2.78 (s, 3H), 0.98 (t, J=7.1 Hz, 3H); MS (ES+): 436.0 (M+1).

### Step-2: Preparation of ethyl 2-(2-((7-(2-(1-((tert-butylsulfinyl)imino)ethyl)thiazol-4-yl)benzofuran-5-yl)methoxy)phenyl)acetate (7b)

Compound 7b was prepared according to procedure reported in Step-5 of scheme 1 from ethyl 2-(2-((7-(2-acetylthiazol-4-yl)benzofuran-5-yl)methoxy)phenyl)acetate (7a) (950 mg, 2.181 mmol) in THE (10 mL) using (R)-2-methylpropane-2-sulfinamide (529 mg, 4.36 mmol), tetraethoxytitanium (0.915 mL, 4.36 mmol) and stirring at room temperature for 18 h. This gave after workup and purification by flash column chromatography [(silica gel, (40 g), eluting with ethyl acetate in hexanes (0 to 20%)]ethyl 2-(2-((7-(2-(1-((tert-butylsulfinyl)imino)ethyl)thiazol-4-yl)benzofuran-5-yl)methoxy)phenyl)acetate (7b) (714 mg, 61% yield) as brown syrup; ¹H NMR (300 MHz, DMSO-d₆) δ 8.60 (s, 1H), 8.21-8.18 (m, 1H), 7.77 (d, J=1.7 Hz, 1H), 7.28 (dd, J=7.8, 1.8 Hz, 1H), 7.26-7.21 (m, 2H), 7.14 (d, J=8.7 Hz, 1H), 7.12 (d, J=2.2 Hz, 1H), 6.92 (td, J=7.4, 1.1 Hz, 1H), 5.27 (s, 2H), 4.01-3.91 (m, 2H), 3.64 (s, 2H), 2.90 (s, 3H), 1.28 (s, 9H), 0.98 (t, J=7.1 Hz, 3H); MS (ES+): 539.1 (M+1).

### Step-3: Preparation of ethyl 2-(2-((7-(2-(1-((R)-1,1-dimethylethylsulfinamido)ethyl)thiazol-4-yl)benzofuran-5-yl)methoxy)phenyl)acetate (7c)

Compound 7c was prepared according to procedure reported in Step-3 of scheme 5 from ethyl 2-(2-((7-(2-(1-((tert-butylsulfinyl)imino)ethyl)thiazol-4-yl)benzofuran-5-yl)methoxy)phenyl)acetate (7b) (714 mg, 1.325 mmol) in THE (10 mL) and water (1 mL) using sodium borohydride (150 mg, 3.98 mmol). This gave after workup and purification by flash column chromatography [(silica gel (25 g), eluting with ethyl acetate in hexanes (0 to 100%)]ethyl 2-(2-((7-(2-(1-((R)-1,1-dimethylethylsulfinamido)ethyl)thiazol-4-yl)benzofuran-5-yl)methoxy)phenyl)acetate (7c) (712 mg, 99% yield) as a white solid; MS (ES+): 541.2 (M+1)

### Step-4: Preparation of ethyl 2-(2-((7-(2-(1-aminoethyl)thiazol-4-yl)benzofuran-5-yl)methoxy)phenyl)acetate (7d)

To a solution of ethyl 2-(2-((7-(2-(1-((R)-1,1-dimethylethylsulfinamido)ethyl)thiazol-4-yl)benzofuran-5-yl)methoxy)phenyl)acetate (7c) (715 mg, 1.322 mmol) in tetrahydrofuran (3 mL) was added aqueous 2N HCl (1.984 mL, 3.97 mmol) and stirred at RT for 4 h. The reaction mixture was concentrated to dryness to afford ethyl 2-(2-((7-(2-(1-aminoethyl)thiazol-4-yl)benzofuran-5-yl)methoxy)phenyl)acetate (7d) which was used as such in the next step without further purification; MS (ES+): 437.1 (M+1).

### Step-5: Preparation of 2-(2-((7-(2-(1-aminoethyl)thiazol-4-yl)benzofuran-5-yl)methoxy)phenyl)acetic acid (7e)

Compound 7e was prepared according to procedure reported in Step-8 of scheme 1 from ethyl 2-(2-((7-(2-(1-aminoethyl)thiazol-4-yl)benzofuran-5-yl)methoxy)phenyl)acetate (7d) (554 mg, 1.269 mmol) in THE (5 mL), methanol (1 mL) and water (1 mL) using lithium hydroxide monohydrate (213 mg, 5.08 mmol) and stirring for 10 h at room temperature. This gave after workup and purification by reverse-phase column chromatography [C-18 column (40 g), eluting with 0.1% aq HCl in water and acetonitrile from 0-100%]2-(2-((7-(2-(1-aminoethyl)thiazol-4-yl)benzofuran-5-yl)methoxy)phenyl)acetic acid (7e) (193 mg, 37% yield) hydrochloride salt as a white solid. ¹H NMR (300 MHz, DMSO-d₆) δ 12.18 (s, 1H), 8.77 (s, 3H), 8.35 (s, 1H), 8.16 (d, J=1.7 Hz, 1H), 8.12 (d, J=2.2 Hz, 1H), 7.72 (d, J=1.7 Hz, 1H), 7.23-7.12 (m, 2H), 7.06-6.98 (m, 2H), 6.84 (t, J=7.4, 1.1 Hz, 1H), 5.20 (s, 2H), 4.96-4.80 (m, 1H), 3.54 (s, 2H), 1.64 (d, J=6.8 Hz, 3H). MS (ES+): 409.1 (M+1); Analysis calculated for C₂₂H₂₀N₂O₄S·HCl·H₂O: C, 57.08; H, 5.01; Cl, 7.66; N, 6.05; Found: C, 56.88; H, 5.02; Cl, 7.56; N, 6.00.

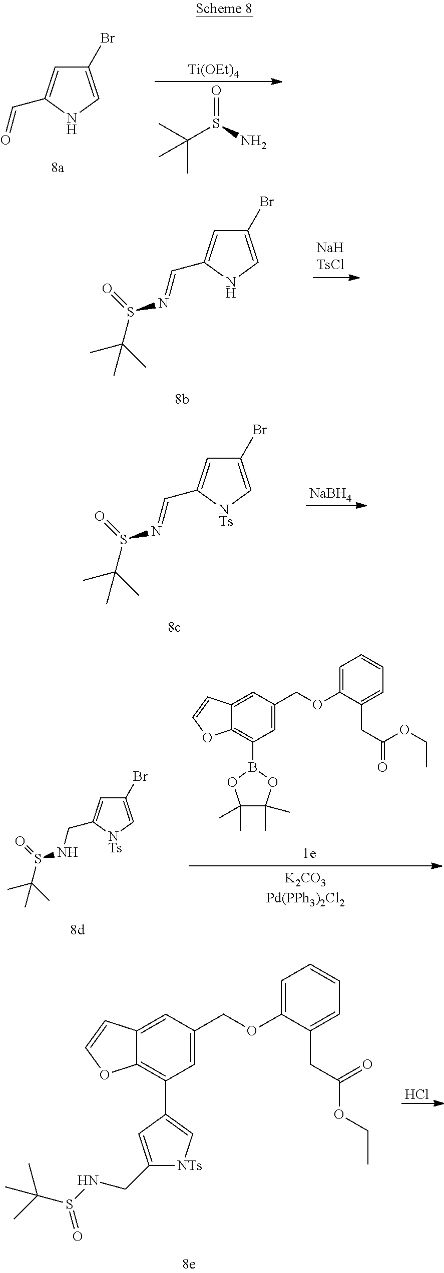

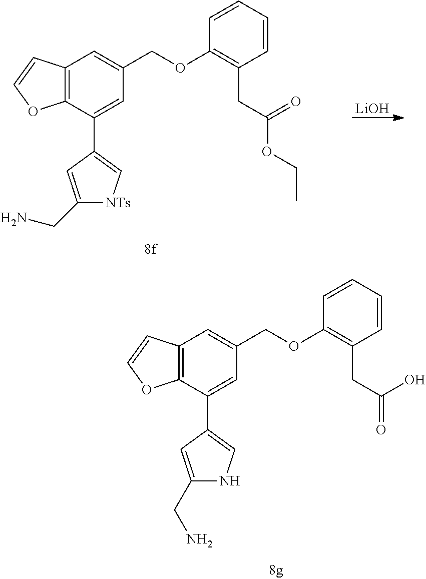 [SMILES: CCOC(=O)Cc1ccccc1OCc1cc(-c2cc(CN)n(S(=O)(=O)c3ccc(C)cc3)c2)c2occc2c1 ; "LiOH" ; NCc1cc(-c2cc(COc3ccccc3CC(=O)O)cc3ccoc23)c[nH]1]

### Preparation of 2-(2-((7-(5-(aminomethyl)-1H-pyrrol-3-yl)benzofuran-5-yl)methoxy)phenyl)acetic acid (8g)

### Step-1: Preparation of N-((4-bromo-1H-pyrrol-2-yl)methylene)-2-methylpropane-2-sulfinamide (8b)

Compound 8b was prepared according to procedure reported in Step-5 of scheme 1 from 4-bromo-1H-pyrrole-2-carbaldehyde (8a) (1 g, 5.75 mmol) in THE (30 mL) using (R)-2-methylpropane-2-sulfinamide (1.393 g, 11.49 mmol), tetraethoxytitanium (2.410 mL, 11.49 mmol) and stirring at room temperature for 18 h. This gave after workup and purification by flash column chromatography [(silica gel (40 g), eluting with ethyl acetate in hexanes (0 to 20%)]N-((4-bromo-1H-pyrrol-2-yl)methylene)-2-methylpropane-2-sulfinamide (8b) (1.37 g, 4.94 mmol, 86% yield) as brown syrup; ¹H NMR (300 MHz, DMSO-d₆) δ 12.16 (s, 1H), 8.22 (s, 1H), 7.30 (d, J=1.6 Hz, 1H), 6.95 (d, J=1.6 Hz, 1H), 1.15 (s, 9H); MS (ES+): 278.9 (M+1).

### Step-2: Preparation of N-((4-bromo-1-tosyl-1H-pyrrol-2-yl)methylene)-2-methylpropane-2-sulfinamide (8c)

To a solution of N-((4-bromo-1H-pyrrol-2-yl)methylene)-2-methylpropane-2-sulfinamide (8b) (650 mg, 2.345 mmol) in DMF (5 mL) at 0° C. was added sodium hydride (281 mg, 7.04 mmol) stirred for 15 min followed by the addition of 4-methylbenzene-1-sulfonyl chloride (1341 mg, 7.04 mmol) at 0° C. The reaction mixture was allowed to warm to RT overnight and poured into EtOAc. The reaction mixture was acidified with 3 M HCl, washed with water, brine, dried, filtered and concentrated in vacuum. The residue obtained was purified by flash column chromatography [silica (80 g), eluting with EtOAc:MeOH (9:1) in Hexane from 0-100%] to give N-((4-bromo-1-tosyl-1H-pyrrol-2-yl)methylene)-2-methylpropane-2-sulfinamide (8c) (222 mg, 22% yield) as a yellow solid; ¹H NMR (300 MHz, DMSO-d₆) δ 8.76 (s, 1H), 8.04 (d, J=1.8 Hz, 1H), 7.86-7.76 (m, 2H), 7.51 (d, J=8.1 Hz, 2H), 7.27 (d, J=1.8 Hz, 1H), 2.39 (s, 3H), 1.10 (s, 9H); MS (ES+): 432.9 (M+1).

### Step-3: Preparation of N-((4-bromo-1-tosyl-1H-pyrrol-2-yl)methyl)-2-methylpropane-2-sulfinamide (8d)

Compound 8d was prepared according to procedure reported in Step-3 of scheme 5 from N-((4-bromo-1-tosyl-1H-pyrrol-2-yl)methylene)-2-methylpropane-2-sulfinamide (8c) (222 mg, 0.515 mmol) in THE (3 mL) and water (0.3 mL) using sodium borohydride (58.4 mg, 1.544 mmol). This gave after workup and purification by flash column chromatography [(silica gel (25 g), eluting with ethyl acetate in hexanes (0 to 100%)]N-((4-bromo-1-tosyl-1H-pyrrol-2-yl)methyl)-2-methylpropane-2-sulfinamide (8d) (200 mg, 90% yield) as a white solid; MS(ES+): 434.9 (M+1).

### Step-4: Preparation of ethyl 2-(2-((7-(5-((1,1-dimethylethylsulfinamido)methyl)-1-tosyl-1H-pyrrol-3-yl)benzofuran-5-yl)methoxy)phenyl)acetate (8e)

Compound 8e was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (224 mg, 0.512 mmol) in dioxane (12 mL) using N-((4-bromo-1-tosyl-1H-pyrrol-2-yl)methyl)-2-methylpropane-2-sulfinamide (8d) (222 mg, 0.512 mmol), bis(triphenylphosphine)Palladium(II)chloride [Pd(PPh₃)₂Cl₂](53.9 mg, 0.077 mmol), a solution of K₂CO₃ (212 mg, 1.537 mmol) in water (4 mL) and heating at 90° C. for 3 h on oil bath. This gave after workup and purification by flash column chromatography [silica gel (12 g), eluting with DMA-80 in DCM from 0-50%]ethyl 2-(2-((7-(5-((1,1-dimethylethylsulfinamido)methyl)-1-tosyl-1H-pyrrol-3-yl)benzofuran-5-yl)methoxy)phenyl)acetate (8e) (208 mg, 61% yield) as a yellow syrup; ¹H NMR (300 MHz, DMSO-d₆) δ 8.15 (d, J=2.2 Hz, 1H), 8.10 (d, J=1.9 Hz, 1H), 7.91-7.87 (m, 2H), 7.86 (s, 1H), 7.67-7.65 (m, 2H), 7.48 (d, J=8.1 Hz, 2H), 7.23 (dd, J=7.6, 3.8 Hz, 2H), 7.10 (d, J=8.0 Hz, 1H), 7.04 (d, J=2.2 Hz, 1H), 7.00 (d, J=1.8 Hz, 1H), 6.91 (t, J=7.3 Hz, 1H), 5.21 (s, 2H), 4.31-4.20 (m, 2H), 3.96 (q, J=7.1 Hz, 2H), 3.62 (s, 2H), 2.38 (s, 3H), 1.12 (s, 9H), 0.97 (t, J=7.1 Hz, 3H); MS (ES+): 663.0 (M+1).

### Step-5: Preparation of ethyl 2-(2-((7-(5-(aminomethyl)-1-tosyl-1H-pyrrol-3-yl)benzofuran-5-yl)methoxy)phenyl)acetate (8f)

To a solution of ethyl 2-(2-((7-(5-((1,1-dimethylethylsulfinamido)methyl)-1-tosyl-1H-pyrrol-3-yl)benzofuran-5-yl)methoxy)phenyl)acetate (8e) (208 mg, 0.314 mmol) in tetrahydrofuran (0.5 mL) was added aqueous 2N HCl (0.471 mL, 0.941 mmol) and stirred at RT for 4 h. The reaction mixture was concentrated to dryness to afford ethyl 2-(2-((7-(5-(aminomethyl)-1-tosyl-1H-pyrrol-3-yl)benzofuran-5-yl)methoxy)phenyl)acetate (8f) which was used as such in the next step without further purification; (ES+): 559.2 (M+1).

### Step-6: Preparation of 2-(2-((7-(5-(aminomethyl)-1H-pyrrol-3-yl)benzofuran-5-yl)methoxy)phenyl)acetic acid (8g)

Compound 8g was prepared according to procedure reported in Step-8 of scheme 1 from ethyl 2-(2-((7-(5-(aminomethyl)-1-tosyl-1H-pyrrol-3-yl)benzofuran-5-yl)methoxy)phenyl)acetate (8f) (166 mg, 0.297 mmol) in THE (1 mL), methanol (0.1 mL) and water (0.1 mL) using lithium hydroxide monohydrate (28.5 mg, 1.189 mmol) and stirring for 10 h at room temperature. This gave after workup and purification by reverse-phase column chromatography [C-18 column (40 g), eluting with 0.1% aq HCl in water and acetonitrile from 0-100%]2-(2-((7-(5-(aminomethyl)-1H-pyrrol-3-yl)benzofuran-5-yl)methoxy)phenyl)acetic acid (8g) (25 mg, 22%) hydrochloride salt as a light pink solid. ¹H NMR (300 MHz, DMSO-d₆) δ 12.21 (bs, 1H), 11.20 (bs, 1H), 8.17 (s, 3H), 8.07 (d, J=2.2 Hz, 1H), 7.59 (s, 2H), 7.51 (s, 1H), 7.22 (d, J=7.4 Hz, 2H), 7.08 (d, J=8.3 Hz, 1H), 6.98 (d, J=2.2 Hz, 1H), 6.91 (d, J=7.3 Hz, 1H), 6.87 (s, 1H), 5.22 (s, 2H), 4.05 (s, 2H), 3.60 (s, 2H). MS (ES+): 377.1 (M+1); Analysis calculated for C₂₂H₂₀N₂O₄.1.1HCl·2.25H₂O: C, 57.81; H, 5.65; Cl, 8.53; N, 6.13; found: C, 57.77; H, 5.29; Cl, 8.97; N, 6.01.

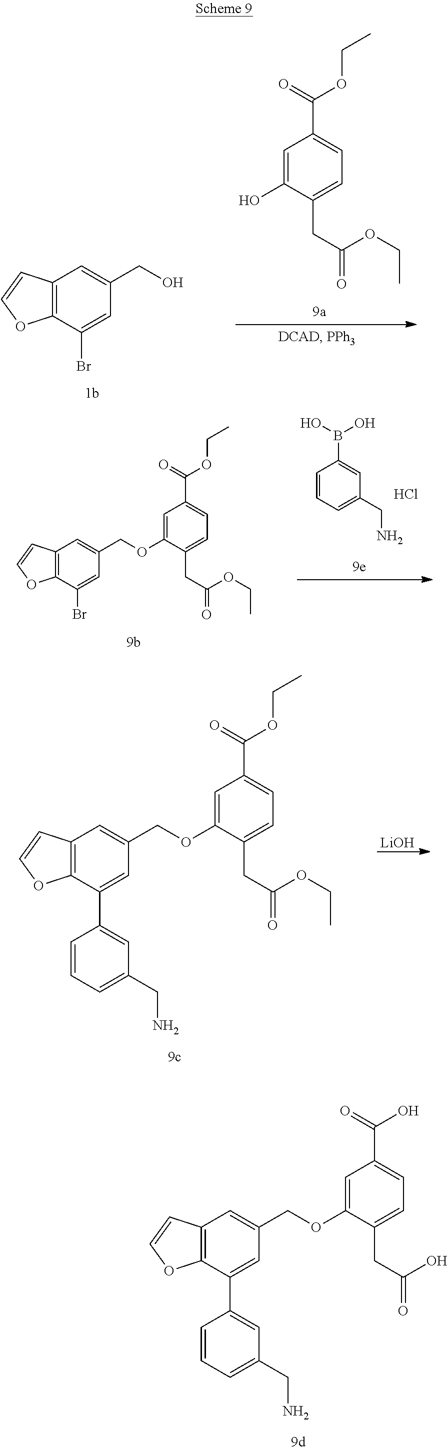

### Preparation of 3-((7-(3-(aminomethyl)phenyl)benzofuran-5-yl)methoxy)-4-(carboxymethyl)benzoic acid (9d)

### Step-1: Preparation of ethyl 3-((7-bromobenzofuran-5-yl)methoxy)-4-(2-ethoxy-2-oxoethyl)benzoate (9b)

Compound 9b was prepared according to procedure reported in Step-2 of scheme 1 from (7-bromobenzofuran-5-yl)methanol (1b) (191 mg, 0.842 mmol) in DCM (10 mL) using triphenylphosphine (265 mg, 1.011 mmol) and ethyl 4-(2-ethoxy-2-oxoethyl)-3-hydroxybenzoate (9a) (170 mg, 0.674 mmol), a solution of bis(4-chlorobenzyl)azodicarboxylate (DCAD) (371 mg, 1.011 mmol) in DCM (10 mL). This gave after workup and purification by flash column chromatography [(silica gel, eluting with hexanes/ethyl acetate (1:0 to 3:1)]ethyl 3-((7-bromobenzofuran-5-yl)methoxy)-4-(2-ethoxy-2-oxoethyl)benzoate (9b) (217 mg, 70%) as a white solid; ¹H NMR (300 MHz, DMSO-d₆) δ 8.15 (d, J=2.1 Hz, 1H), 7.74 (d, J=1.4 Hz, 1H), 7.63 (d, J=1.5 Hz, 1H), 7.59 (d, J=1.5 Hz, 1H), 7.58-7.54 (m, 1H), 7.39 (d, J=7.7 Hz, 1H), 7.12 (d, J=2.2 Hz, 1H), 5.26 (s, 2H), 4.31 (q, J=7.1 Hz, 2H), 4.02 (q, J=7.1 Hz, 2H), 3.72 (s, 2H), 1.32 (t, J=7.1 Hz, 3H), 1.07 (t, J=7.1 Hz, 3H); MS (ES+): 483.00 & 485.00 (M+Na).

### Step-2: Preparation of ethyl 3-((7-(3-(aminomethyl)phenyl)benzofuran-5-yl)methoxy)-4-(2-ethoxy-2-oxoethyl)benzoate (9c)

Compound 9c was prepared according to procedure reported in Step-4 of scheme 1 from ethyl 3-((7-bromobenzofuran-5-yl)methoxy)-4-(2-ethoxy-2-oxoethyl)benzoate (9b) (202 mg, 0.438 mmol) in dioxane (12 mL) using 3-(aminomethyl)phenylboronic acid hydrochloride (9e) (123 mg, 0.657 mmol), bis(triphenylphosphine)Palladium(II)chloride [Pd(PPh₃)₂Cl₂](61.5 mg, 0.088 mmol), a solution of K₂CO₃ (182 mg, 1.314 mmol) in water (1.5 mL) and heating at 100° C. for 3 h on oil bath. This gave after workup and purification by flash column chromatography [silica gel with dichloromethane/methanol (1:0 to 19:1)]ethyl 3-((7-(3-(aminomethyl)phenyl)benzofuran-5-yl)methoxy)-4-(2-ethoxy-2-oxoethyl)benzoate (9c) (126 mg, 59% yield) as a colorless gum; ¹H NMR (300 MHz, DMSO-d₆) δ 8.10 (d, J=2.2 Hz, 1H), 7.83 (s, 1H), 7.76-7.69 (m, 2H), 7.64 (d, J=1.5 Hz, 1H), 7.61 (d, J=1.6 Hz, 1H), 7.55 (dd, J=7.7, 1.4 Hz, 1H), 7.47 (t, J=7.6 Hz, 1H), 7.43-7.36 (m, 2H), 7.06 (d, J=2.2 Hz, 1H), 5.33 (s, 2H), 4.31 (q, J=7.1 Hz, 2H), 3.94 (q, J=7.1 Hz, 2H), 3.82 (s, 2H), 3.72 (s, 2H), 1.31 (t, J=7.1 Hz, 3H), 0.98 (t, J=7.1 Hz, 3H); MS (ES+): 488.20 (M+1).

### Step-3: Preparation of 3-((7-(3-(aminomethyl)phenyl)benzofuran-5-yl)methoxy)-4-(carboxymethyl)benzoic acid (9d)

Compound 9d was prepared according to procedure reported in Step-8 of scheme 1 from ethyl 3-((7-(3-(aminomethyl)phenyl)benzofuran-5-yl)methoxy)-4-(2-ethoxy-2-oxoethyl)benzoate (9c) (120 mg, 0.246 mmol) in THE (5 mL), methanol (5 mL) using a solution of lithium hydroxide monohydrate (105 mg, 2.461 mmol) in water (5 mL) and stirring for 18 h at room temperature. This gave after workup and purification by reverse-phase column chromatography [C-18 column, eluting with 0.1% aq HCl in water and acetonitrile from 0-100%]3-((7-(3-(aminomethyl)phenyl)benzofuran-5-yl)methoxy)-4-(carboxymethyl)benzoic acid (9d) (65 mg, 61%) hydrochloride salt as a white solid; ¹H NMR (300 MHz, DMSO-d₆) δ 8.11 (d, J=2.2 Hz, 1H), 8.01 (s, 1H), 7.94 (dt, J=7.3, 1.8 Hz, 1H), 7.77 (d, J=1.6 Hz, 1H), 7.68 (d, J=1.6 Hz, 1H), 7.64-7.49 (m, 4H), 7.36 (d, J=7.7 Hz, 1H), 7.07 (d, J=2.2 Hz, 1H), 5.34 (s, 2H), 4.13 (s, 2H), 3.68 (s, 2H); MS (ES+): 432.05 (M+1); Analysis calculated for C₂₅H₂₁NO₆·HCl·1.75H₂O: C, 60.12H, 5.15; N, 2.80; Cl, 7.10; Found: C, 60.23; H, 5.17; N, 2.96; Cl, 7.14.

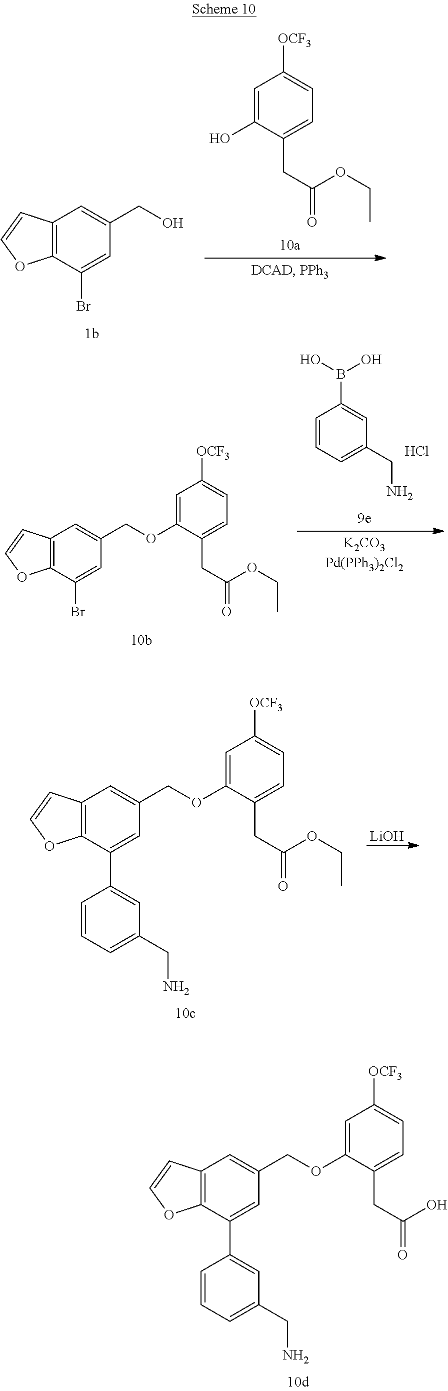

### Preparation of 2-(2-((7-(3-(aminomethyl)phenyl)benzofuran-5-yl)methoxy)-4-(trifluoromethoxy)phenyl)acetic acid (10d)

### Step-1: Preparation of ethyl 2-(2-((7-bromobenzofuran-5-yl)methoxy)-4-(trifluoromethoxy)phenyl)acetate (10b)

Compound 10b was prepared according to procedure reported in Step-2 of scheme 1 from (7-bromobenzofuran-5-yl)methanol (1b) (173 mg, 0.762 mmol) in DCM (10 mL) using triphenylphosphine (240 mg, 0.914 mmol) and ethyl 2-(2-hydroxy-4-(trifluoromethoxy)phenyl)acetate (10a) (161 mg, 0.609 mmol; CAS #1261673-71-5), a solution of bis(4-chlorobenzyl)azodicarboxylate (DCAD) (336 mg, 0.914 mmol) in DCM (10 mL). This gave after workup and purification by flash column chromatography [(silica gel, eluting with hexanes/ethyl acetate (1:0 to 4:1)]ethyl 2-(2-((7-bromobenzofuran-5-yl)methoxy)-4-(trifluoromethoxy)phenyl)acetate (10b) (180 mg, 62%) as a white solid; ¹H NMR (300 MHz, DMSO-d₆) δ 8.15 (d, J=2.2 Hz, 1H), 7.72 (d, J=1.5 Hz, 1H), 7.61 (d, J=1.5 Hz, 1H), 7.35 (d, J=8.3 Hz, 1H), 7.15-7.10 (m, 2H), 6.96-6.90 (m, 1H), 5.22 (s, 2H), 4.01 (q, J=7.2 Hz, 2H), 3.64 (s, 2H), 1.06 (t, J=7.1 Hz, 3H); ¹⁹F NMR (282 MHz, DMSO-d₆) δ −56.67; MS (ES+): 494.90 & 496.90 (M+Na).

### Step-2: Preparation of ethyl 2-(2-((7-(3-(aminomethyl)phenyl)benzofuran-5-yl)methoxy)-4-(trifluoromethoxy)phenyl)acetate (10c)

Compound 10c was prepared according to procedure reported in Step-4 of scheme 1 from ethyl 2-(2-((7-bromobenzofuran-5-yl)methoxy)-4-(trifluoromethoxy)phenyl)acetate (10b) (170 mg, 0.359 mmol) in dioxane (12 mL) using 3-(aminomethyl)phenylboronic acid hydrochloride (9e) (101 mg, 0.539 mmol), bis(triphenylphosphine)Palladium(II)chloride [Pd(PPh₃)₂Cl₂](50.4 mg, 0.072 mmol), a solution of K₂CO₃ (149 mg, 1.078 mmol) in water (1.5 mL) and heating at 100° C. for 11 h on oil bath. This gave after workup and purification by flash column chromatography [silica gel with dichloromethane/methanol (1:0 to 9:1)]ethyl 2-(2-((7-(3-(aminomethyl)phenyl)benzofuran-5-yl)methoxy)-4-(trifluoromethoxy)phenyl)acetate (10c) (115 mg, 64% yield) as a colorless gum; ¹H NMR (300 MHz, DMSO-d₆) δ 8.10 (d, J=2.2 Hz, 1H), 7.83-7.80 (m, 1H), 7.75-7.67 (m, 2H), 7.58 (d, J=1.7 Hz, 1H), 7.46 (t, J=7.5 Hz, 1H), 7.40 (d, J=7.7 Hz, 1H), 7.35 (d, J=8.3 Hz, 1H), 7.17 (d, J=2.3 Hz, 1H), 7.06 (d, J=2.2 Hz, 1H), 6.96-6.89 (m, 1H), 5.28 (s, 2H), 3.91 (q, J=7.1 Hz, 2H), 3.81 (s, 2H), 3.65 (s, 2H), 0.96 (t, J=7.1 Hz, 3H); ¹⁹F NMR (282 MHz, DMSO-d₆) δ −56.64; MS (ES+): 500.00 (M+1).

### Step-3: Preparation of 2-(2-((7-(3-(aminomethyl)phenyl)benzofuran-5-yl)methoxy)-4-(trifluoromethoxy)phenyl)acetic acid (10d)

Compound 10d was prepared according to procedure reported in Step-8 of scheme 1 from ethyl 2-(2-((7-(3-(aminomethyl)phenyl)benzofuran-5-yl)methoxy)-4-(trifluoromethoxy)phenyl)acetate (10c) (110 mg, 0.220 mmol) in THE (5 mL), methanol (5 mL) using a solution of lithium hydroxide monohydrate (56.6 mg, 1.321 mmol) in water (5 mL) and stirring for 18 h at room temperature. This gave after workup and purification by reverse-phase column chromatography [C-18 column, eluting with 0.1% aq HCl in water and acetonitrile from 0-100%]2-(2-((7-(3-(aminomethyl)phenyl)benzofuran-5-yl)methoxy)-4-(trifluoromethoxy)phenyl)acetic acid (10d) (68 mg, 66%) hydrochloride salt as a white solid; ¹H NMR (300 MHz, DMSO-d₆) δ 8.11 (d, J=2.2 Hz, 1H), 8.02-7.99 (m, 1H), 7.92 (dt, J=7.4, 1.7 Hz, 1H), 7.77 (d, J=1.6 Hz, 1H), 7.65 (d, J=1.6 Hz, 1H), 7.63-7.52 (m, 2H), 7.34 (d, J=8.3 Hz, 1H), 7.14 (d, J=2.3 Hz, 1H), 7.08 (d, J=2.2 Hz, 1H), 6.96-6.87 (m, 1H), 5.30 (s, 2H), 4.13 (s, 2H), 3.61 (s, 2H); ¹⁹F NMR (282 MHz, DMSO-d₆) δ −56.65; MS (ES+): 472.00 (M+1); MS (ES−): 470.00 (M−1); Analysis calculated for C₂₅H₂₀F₃NO₅·HCl·0.75H₂O: C, 57.59; H, 4.35; Cl, 6.80; N, 2.69; Found: C, 57.69; H, 4.25; Cl, 6.68; N, 2.70.

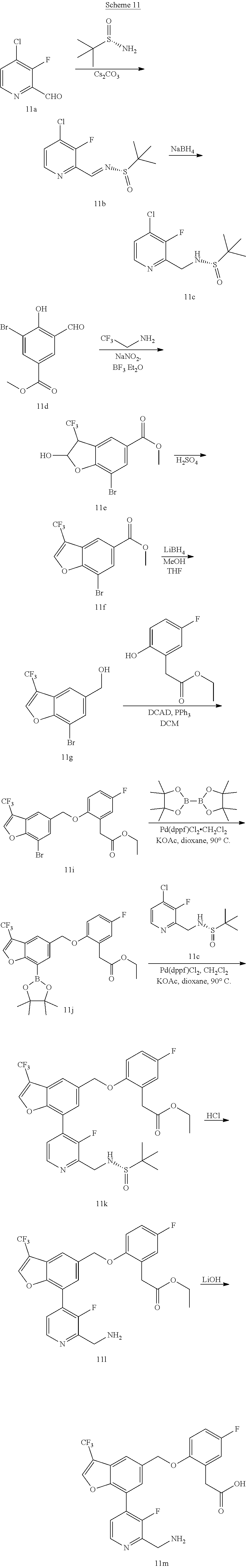

### Preparation of 2-(2-((7-(2-(aminomethyl)-3-fluoropyridin-4-yl)-3-(trifluoromethyl)benzofuran-5-yl)methoxy)-5-fluorophenyl)acetic acid (11m)

### Step-1: Preparation of (S)—N-((4-chloro-3-fluoropyridin-2-yl)methylene)-2-methylpropane-2-sulfinamide (11b)

To a solution of 4-chloro-3-fluoropicolinaldehyde (11a) (6.73 g, 42.2 mmol; CAS #1260878-78-1) and Cs₂CO₃ (27.5 g, 84 mmol) in DCM (350 mL) was added (S)-2-methylpropane-2-sulfinamide (5.88 g, 48.5 mmol) and stirred at room temperature for 1 h. The reaction mixture was diluted with DCM and washed with brine (3×200 mL). The organic layer was dried, filtered and concentrated in vacuo to afford (S)—N-((4-chloro-3-fluoropyridin-2-yl)methylene)-2-methylpropane-2-sulfinamide (11b) (11.08 g, 100% yield) which was used in the next reaction without further purification; ¹H NMR (300 MHz, DMSO-d₆) δ 8.59 (d, J=5.1 Hz, 1H), 8.56 (s, 1H), 7.97 (dd, J=5.6, 5.0 Hz, 1H), 1.21 (s, 9H).

### Step-2: Preparation of (+)-N-((4-chloro-3-fluoropyridin-2-yl)methyl)-2-methylpropane-2-sulfinamide (11c)

To a solution of (S)—N-((4-chloro-3-fluoropyridin-2-yl)methylene)-2-methylpropane-2-sulfinamide (11b) (11.08 g, 42.2 mmol) in methanol (211 mL) at 0° C. was added NaBH₄ (1.595 g, 42.2 mmol) and stirred for 0.5 h. The reaction was quenched with acetone (20 mL) and concentrated in vacuum. The residue was taken in EtOAc and saturated aqueous NH₄Cl. The organic layer was separated, washed with brine, dried, filtered and concentrated under vacuum. The residue obtained was purified by flash column chromatography [silica gel (120 g), eluting with a 9:1 mixture of ethyl acetate and methanol in hexanes] to afford (+)-N-((4-chloro-3-fluoropyridin-2-yl)methyl)-2-methylpropane-2-sulfinamide (11c) (9.34 g, 84% yield) as a thick clear syrup; ¹H NMR (300 MHz, DMSO-d₆) δ 8.36 (d, J=5.2 Hz, 1H), 7.72-7.62 (m, 1H), 5.86 (t, J=5.9 Hz, 1H), 4.34 (dd, J=5.9, 2.2 Hz, 2H), 1.09 (s, 9H); Optical rotation [α]D=+53.88 (c=0.49, MeOH).

### Step-3: Preparation of methyl 7-bromo-2-hydroxy-3-(trifluoromethyl)-2,3-dihydrobenzofuran-5-carboxylate (11e)

To a solution of 2,2,2-trifluoroethanamine HCl (1.71 g, 17.26 mmol; CAS #373-88-6) in DCM (15 mL) at 0° C., was added a solution of sodium nitrite (1.27 g, 18.41 mmol) in water (1.5 mL). The mixture was kept at ice bath for 1 h, cooled to −78° C. and added methyl 3-bromo-5-formyl-4-hydroxybenzoate (11d) (0.522 g, 2.015 mmol; CAS #706820-79-3) and boron trifluoride etherate (1.2 mL, 9.47 mmol) was added. The mixture was stirred at −78° C. for 12 h, warmed to room temperature over a 12 h period. The reaction was quenched with methanol (8 mL), diluted with saturated aqueous NaHCO₃, and extracted with ethyl acetate. The organic layers were combined, washed with brine, dried, filtered and concentrated in vacuum. The residue obtained was purified by flash column chromatography [silica (24g), eluting with ethyl acetate/hexanes, 0-30%] to afford methyl 7-bromo-2-hydroxy-3-(trifluoromethyl)-2,3-dihydrobenzofuran-5-carboxylate (11e) (503 mg, 73% yield) as white solid; ¹H NMR (300 MHz, DMSO-d₆) δ 8.60 (d, J=6.0 Hz, 1H), 8.11 (dd, J=1.7, 0.6 Hz, 1H), 7.91 (dt, J=1.8, 0.9 Hz, 1H), 6.28 (dd, J=5.9, 2.4 Hz, 1H), 4.67-4.46 (m, 1H), 3.85 (s, 3H); ¹⁹F NMR (282 MHz, DMSO-d₆) δ −69.45.

### Step-4: Preparation of methyl 7-bromo-3-(trifluoromethyl)benzofuran-5-carboxylate (11f)

A solution of methyl 7-bromo-2-hydroxy-3-(trifluoromethyl)-2,3-dihydrobenzofuran-5-carboxylate (11e) (495 mg, 1.451 mmol) in sulfuric acid (5 mL, 94 mmol) was stirred at room temperature for 30 min. The mixture was poured into ice water and the white solid obtained was collected by filtration, dried in vacuum to provide methyl 7-bromo-3-(trifluoromethyl)benzofuran-5-carboxylate (11f) (462 mg, 99% yield). ¹H NMR (300 MHz, DMSO-d₆) δ 9.11 (t, J=1.7 Hz, 1H), 8.23 (d, J=1.5 Hz, 1H), 8.20 (dt, J=1.5, 0.9 Hz, 1H), 3.92 (s, 3H).

### Step-5: Preparation of (7-bromo-3-(trifluoromethyl)benzofuran-5-yl)methanol (11g)

To a solution of methyl 7-bromo-3-(trifluoromethyl)benzofuran-5-carboxylate (11f) (927 mg, 2.87 mmol) in THF (12 mL) at −78° C. was added LiBH₄ (2.20 mL, 8.80 mmol, 2 M solution in THF) and MeOH (385 μl, 9.52 mmol). The reaction mixture was stirred at RT for 24 h, quenched with saturated aqueous NH₄Cl solution and extracted with EtOAc. The organic layer was washed with brine, dried, filtered and concentrated in vacuum to afford (7-bromo-3-(trifluoromethyl)benzofuran-5-yl)methanol (11g) (836 mg, 99% yield) as a white solid. ¹H NMR (300 MHz, Methanol-d₄) δ 8.41 (d, J=1.7 Hz, 1H), 7.61 (q, J=1.6 Hz, 2H), 4.69 (s, 2H).

### Step-6: Preparation of ethyl 2-(2-((7-bromo-3-(trifluoromethyl)benzofuran-5-yl)methoxy)-5-fluorophenyl)acetate (11i)

Compound 11i was prepared according to procedure reported in Step-2 of scheme 1 from (7-bromo-3-(trifluoromethyl)benzofuran-5-yl)methanol (11g) (1 g, 3.39 mmol) in DCM (35 mL) using triphenylphosphine (0.978 g, 3.73 mmol), ethyl 2-(5-fluoro-2-hydroxyphenyl)acetate (11h) (0.806 g, 4.07 mmol) and a solution of (E)-bis(4-chlorobenzyl) diazene-1,2-dicarboxylate (DCAD) (1.369 g, 3.73 mmol) in DCM (35 mL) and stirring at room temperature for 1 h. This gave after workup and purification by flash column chromatography (silica gel (40 g), eluting with 0 to 20% ethyl acetate in hexanes) ethyl 2-(2-((7-bromo-3-(trifluoromethyl)benzofuran-5-yl)methoxy)-5-fluorophenyl)acetate (11i) (1.16 g, 72.0% yield) as a white solid. ¹H NMR (300 MHz, DMSO-d₆) δ 8.97 (q, J=1.6 Hz, 1H), 7.79 (s, 2H), 7.23-6.98 (m, 3H), 5.23 (s, 2H), 4.02 (q, J=7.1 Hz, 2H), 3.65 (s, 2H), 1.07 (t, J=7.1 Hz, 3H); ¹⁹F NMR (282 MHz, DMSO-d₆) δ −58.23, −123.71.

### Step-7: Preparation of ethyl 2-(5-fluoro-2-((7-(4,4,5,5-tetramethyl-1,3,2-dioxaborolan-2-yl)-3-(trifluoromethyl)benzofuran-5-yl)methoxy)phenyl)acetate (11j)

Compound 11j was prepared according to procedure reported in Step-3 of scheme 1 from ethyl 2-(2-((7-bromo-3-(trifluoromethyl)benzofuran-5-yl)methoxy)-5-fluorophenyl)acetate (11i) (1.8 g, 3.79 mmol) in anhydrous dioxane (30 mL) using bis(pinacolato)diboron (1.443 g, 5.68 mmol), potassium acetate (1.115 g, 11.36 mmol), Pd(dppf)Cl₂—CH₂Cl₂ (0.309 g, 0.379 mmol) and heating at 90° C. for 18 h under an argon atmosphere. This gave after workup and purification by flash column chromatography [silica (80 g), eluting with EtOAc in hexane from 0-15%]ethyl 2-(5-fluoro-2-((7-(4,4,5,5-tetramethyl-1,3,2-dioxaborolan-2-yl)-3-(trifluoromethyl)benzofuran-5-yl)methoxy)phenyl)acetate (11j) (1.87 g, 95% yield) as a pale yellow oil; ¹H NMR (300 MHz, DMSO-d₆) δ 8.87-8.82 (m, 1H), 7.90 (s, 1H), 7.81-7.79 (m, 1H), 7.18-7.04 (m, 3H), 5.21 (s, 2H), 4.10-3.89 (m, 2H), 3.61 (s, 2H), 1.35 (s, 12H), 1.06-1.00 (m, 3H).

### Step-8: Preparation of ethyl 2-(2-((7-(2-((1,1-dimethylethylsulfinamido)methyl)-3-fluoropyridin-4-yl)-3-(trifluoromethyl)benzofuran-5-yl)methoxy)-5-fluorophenyl)acetate (11k)

Compound 11k was prepared according to procedure reported in Step-4 of scheme 1 from ethyl 2-(5-fluoro-2-((7-(4,4,5,5-tetramethyl-1,3,2-dioxaborolan-2-yl)-3-(trifluoromethyl)benzofuran-5-yl)methoxy)phenyl)acetate (11j) (1.00 g, 1.915 mmol) in dioxane (30 mL) using (+)-N-((4-chloro-3-fluoropyridin-2-yl)methyl)-2-methylpropane-2-sulfinamide (11c) (0.507 g, 1.915 mmol), bis(triphenylphosphine)Palladium(II)chloride [Pd(PPh₃)₂Cl₂](0.269 g, 0.383 mmol), a solution of K₂CO₃ (0.794 g, 5.74 mmol) in water (3.6 mL) and heating at 100° C. for 14 h on oil bath. This gave after workup and purification by flash column chromatography [silica gel (24 g), eluting with 0 to 50% MeOH in ethyl acetate (10%) in hexanes]ethyl 2-(2-((7-(2-((1,1-dimethylethylsulfinamido)methyl)-3-fluoropyridin-4-yl)-3-(trifluoromethyl)benzofuran-5-yl)methoxy)-5-fluorophenyl)acetate (11k) (245 mg, 21% yield) as a colorless oil; MS (ES+): 625.20 (M+1).

### Step-9: Preparation of ethyl 2-(2-((7-(2-(aminomethyl)-3-fluoropyridin-4-yl)-3-(trifluoromethyl)benzofuran-5-yl)methoxy)-5-fluorophenyl)acetate (11l)

To a solution of ethyl 2-(2-((7-(2-((1,1-dimethylethylsulfinamido)methyl)-3-fluoropyridin-4-yl)-3-(trifluoromethyl)benzofuran-5-yl)methoxy)-5-fluorophenyl)acetate (11k) (220 mg, 0.352 mmol) in tetrahydrofuran (15 mL) was added aqueous 2N HCl (0.352 mL, 1.057 mmol) and stirred at RT for 2 h. The reaction mixture was concentrated to dryness to afford ethyl 2-(2-((7-(2-(aminomethyl)-3-fluoropyridin-4-yl)-3-(trifluoromethyl)benzofuran-5-yl)methoxy)-5-fluorophenyl)acetate (11l) which was used as such in the next step without further purification.

### Step-10: Preparation of 2-(2-((7-(2-(aminomethyl)-3-fluoropyridin-4-yl)-3-(trifluoromethyl)benzofuran-5-yl)methoxy)-5-fluorophenyl)acetic acid (11m)

Compound 11m was prepared according to procedure reported in Step-8 of scheme 1 from ethyl 2-(2-((7-(2-(aminomethyl)-3-fluoropyridin-4-yl)-3-(trifluoromethyl)benzofuran-5-yl)methoxy)-5-fluorophenyl)acetate (111) (183 mg, 0.352 mmol) in THE (7 mL), methanol (7 mL) using a solution of lithium hydroxide monohydrate (121 mg, 2.82 mmol) in water (7 mL) and stirring for 16 h at room temperature. This gave after workup and purification by reverse-phase column chromatography [C-18 column, eluting with 0.1% aq HCl in water and acetonitrile from 0-100%]2-(2-((7-(2-(aminomethyl)-3-fluoropyridin-4-yl)-3-(trifluoromethyl)benzofuran-5-yl)methoxy)-5-fluorophenyl)acetic acid (11m) (97 mg, 56.0% yield) HCl salt as a white solid; ¹H NMR (300 MHz, DMSO-d₆) δ 12.25 (s, 1H, D₂O exchangeable), 8.96-8.92 (m, 1H), 8.66 (d, J=4.9 Hz, 1H), 8.51 (s, 3H, D₂O exchangeable), 7.99 (s, 1H), 7.82 (t, J=5.3 Hz, 1H), 7.77 (s, 1H), 7.17-7.04 (m, 3H), 5.32 (s, 2H), 4.39 (s, 2H), 3.60 (s, 2H); ¹⁹F NMR (282 MHz, DMSO-d₆) δ −58.01, −123.77, −128.49; MS (ES+): 493.1 (M+1); (ES−): 491.1 (M−1); Analysis calculated for C₂₄H₁₇F₅N₂O₄·HCl·0.25H₂O: C, 54.05; H, 3.50; Cl, 6.65; N, 5.25; Found: C, 54.08; H, 3.50; Cl, 6.61; N, 5.22.

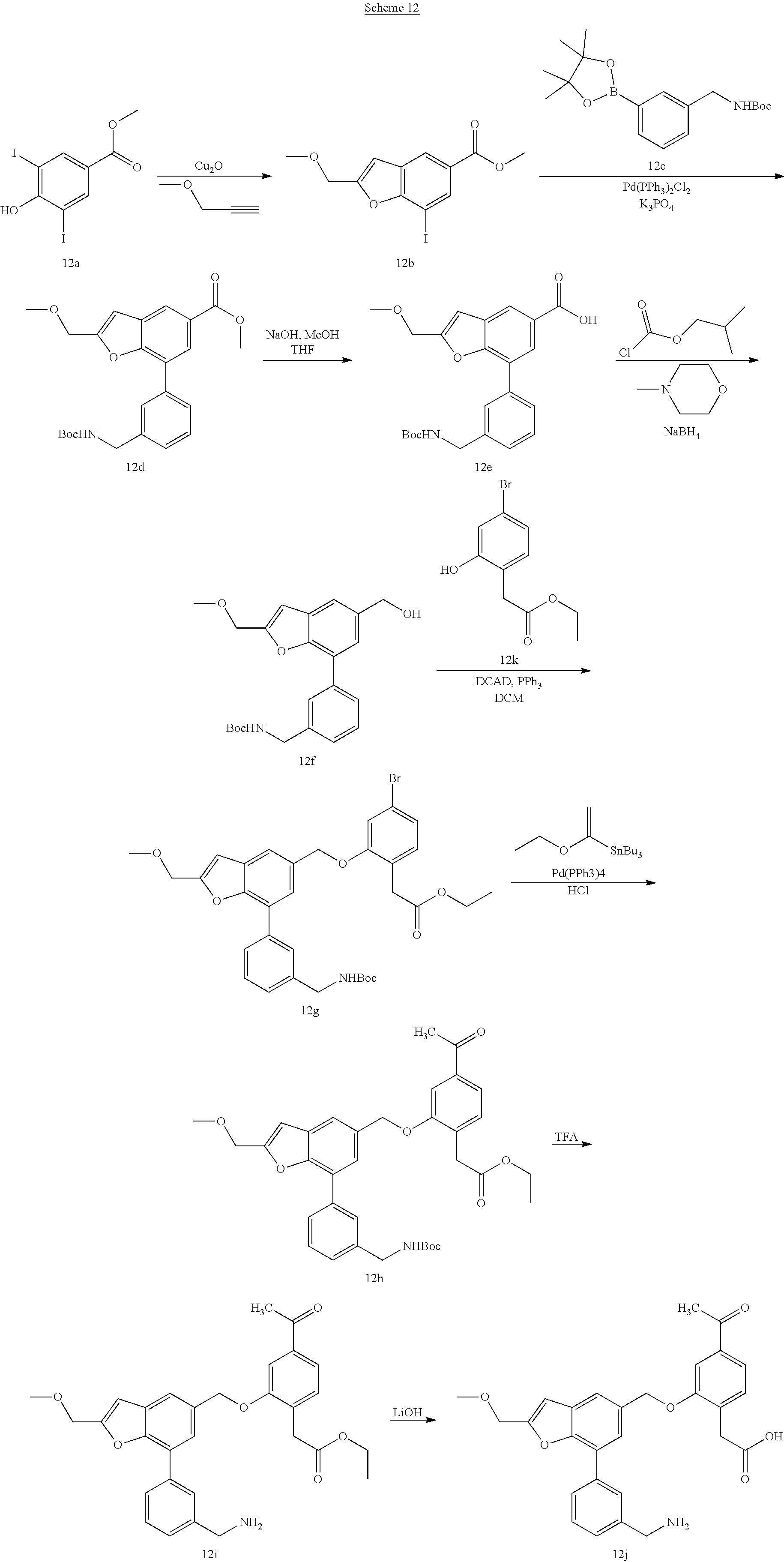

### Preparation of 2-(4-acetyl-2-((7-(3-(aminomethyl)phenyl)-2-(methoxymethyl)benzofuran-5-yl)methoxy)phenyl)acetic acid (12j)

### Step-1: Preparation of methyl 7-iodo-2-(methoxymethyl)benzofuran-5-carboxylate (12b)

To a solution of methyl 4-hydroxy-3,5-diiodobenzoate (12a) (7 g, 17.33 mmol, CAS #3337-66-4) in pyridine (15 mL) was added methyl propargyl ether (1.22 g, 17.33 mmol) and copper(I) oxide (1.24 g, 8.66 mmol). The mixture was degassed, filled with Ar, stirred for 10 min at room temperature and heated at 125° C. for 3h in a sealed flask. The reaction was cooled to room temperature, diluted with EtOAc, washed with 1N aqueous HCl (4×100 mL), water and brine. The organic layer was dried, filtered and concentrated in vacuum. The residue obtained was purified by flash column chromatography [silica (24g), eluting with EtOAc in hexane from 0-70%] to give methyl 7-iodo-2-(methoxymethyl)benzofuran-5-carboxylate (12b) (3.45 g, 58% yield) as a white solid; ¹H NMR (300 MHz, DMSO-d₆) δ 8.27 (d, J=1.5 Hz, 1H), 8.22 (d, J=1.5 Hz, 1H), 7.18 (d, J=0.8 Hz, 1H), 4.60 (s, 2H), 3.88 (s, 3H), 3.36 (s, 3H).

### Step-2: Preparation of methyl 7-(3-(((tert-butoxycarbonyl)amino)methyl)phenyl)-2-(methoxymethyl)benzofuran-5-carboxylate (12d)

Compound 12d was prepared according to the procedure reported in step-4 of scheme 1 from methyl 7-iodo-2-(methoxymethyl)benzofuran-5-carboxylate (12b) (500 mg, 1.44 mmol) in 1,4-dioxane (10.0 mL) using tert-butyl 3-(4,4,5,5-tetramethyl-1,3,2-dioxaborolan-2-yl)benzylcarbamate (12c) (625 mg, 1.87 mmol), Pd(PPh₃)₂Cl₂ (0.15 g, 0.216 mmol), K₃PO₄ (919 mg, 4.33 mmol) and heating under a nitrogen atmosphere at 90° C. for 12 h on an oil bath. This gave after workup, purification by flash column chromatography (silica gel, eluting with 0-30% EtOAc in n-heptane) methyl 7-(3-(((tert-butoxycarbonyl)amino)methyl)phenyl)-2-(methoxymethyl)benzofuran-5-carboxylate (12d) (200 mg, 32%) as an off-white solid; ¹H NMR (300 MHz, DMSO-d₆) δ 8.29 (d, J=1.7 Hz, 1H), 8.03 (d, J=1.7 Hz, 1H), 7.75 (d, J=7.7 Hz, 1H), 7.67 (s, 1H), 7.51 (t, J=7.5 Hz, 2H), 7.33 (d, J=7.6 Hz, 1H), 7.13 (s, 1H), 4.59 (s, 2H), 4.23 (d, J=6.2 Hz, 2H), 3.90 (s, 3H), 3.33 (s, 3H), 1.40 (s, 9H).

### Step-3: Preparation of 7-(3-(((tert-butoxycarbonyl)amino)methyl)phenyl)-2-(methoxymethyl)benzofuran-5-carboxylic acid (12e)

Compound 12e was prepared according to the procedure reported in step-8 of scheme 1 from methyl 7-(3-(((tert-butoxycarbonyl)amino)methyl)phenyl)-2-(methoxymethyl)benzofuran-5-carboxylate (12d) (1.3 g, 3.05 mmol) in THE (13 mL) MeOH (39 mL) using an aqueous solution of sodium hydroxide (13.0 mL, 0.36 g, 9.16 mmol). This gave after workup 7-(3-(((tert-butoxycarbonyl)amino)methyl)phenyl)-2-(methoxymethyl)benzofuran-5-carboxylic acid (12e) (1.2 g, 95.45%) as an off-white solid; ¹H NMR (300 MHz, DMSO-d₆) δ 13.01 (s, 1H), 8.26 (d, J=1.7 Hz, 1H), 8.05 (d, J=1.7 Hz, 1H), 7.76 (d, J=7.7 Hz, 1H), 7.68 (s, 1H), 7.50 (dd, J=8.7, 6.7 Hz, 2H), 7.32 (d, J=7.6 Hz, 1H), 7.11 (s, 1H), 4.59 (s, 2H), 4.23 (d, J=6.2 Hz, 2H), 3.33 (s, 3H), 1.40 (s, 9H).

### Step-4: Preparation of tert-butyl 3-(5-(hydroxymethyl)-2-(methoxymethyl)benzofuran-7-yl)benzylcarbamate (12f)

Compound 12f was prepared according to the procedure reported in step-1 of scheme 1 from 7-(3-(((tert-butoxycarbonyl)amino)methyl)phenyl)-2-(methoxymethyl)benzofuran-5-carboxylic acid (12e) (1.2 g, 2.91 mmol) using N-methylmorpholine (0.35 g, 3.49 mmol) in THE (24 mL), isobutyl chloroformate (0.47 g, 3.49 mmol) and NaBH₄ (0.33 g, 8.74 mmol) in water (10 mL). This gave after workup and purification by flash column chromatography (silica gel, eluting with 0-30% EtOAc in n-heptane) tert-butyl 3-(5-(hydroxymethyl)-2-(methoxymethyl)benzofuran-7-yl)benzylcarbamate (12f)

(1.0 g, 86%) as an oil; ¹H NMR (300 MHz, DMSO-d₆) δ 7.72 (d, J=7.9 Hz, 1H), 7.67 (s, 1H), 7.59-7.53 (m, 1H), 7.53-7.39 (m, 3H), 7.28 (d, J=7.7 Hz, 1H), 6.96 (s, 1H), 5.28 (t, J=5.7 Hz, 1H), 4.63 (d, J=5.5 Hz, 2H), 4.55 (s, 2H), 4.31-4.18 (m, 2H), 3.32 (s, 3H), 1.40 (s, 9H).

### Step-5: Preparation of ethyl 2-(4-bromo-2-((7-(3-(((tert-butoxycarbonyl)amino)methyl)phenyl)-2-(methoxymethyl)benzofuran-5-yl)methoxy)phenyl)acetate (12g)

Compound 12g was prepared according to procedure reported in Step-2 of scheme 1 from tert-butyl 3-(5-(hydroxymethyl)-2-(methoxymethyl)benzofuran-7-yl)benzylcarbamate (12f) (1.2 g, 3.02 mmol) in DCM (30 mL) using triphenylphosphine (1.029 g, 3.92 mmol), ethyl 2-(4-bromo-2-hydroxyphenyl)acetate (12k) (0.782 g, 3.02 mmol) and a solution of (E)-bis(4-chlorobenzyl) diazene-1,2-dicarboxylate (DCAD) (1.441 g, 3.92 mmol) in DCM (30 mL) and stirring at room temperature for 1 h. This gave after workup and purification by flash column chromatography (silica gel (80 g), eluting with 0 to 30% ethyl acetate in hexanes) ethyl 2-(4-bromo-2-((7-(3-(((tert-butoxycarbonyl)amino)methyl)phenyl)-2-(methoxymethyl)benzofuran-5-yl)methoxy)phenyl)acetate (12g) (890 mg, 46% yield). ¹H NMR (300 MHz, DMSO-d₆) δ 7.76-7.68 (m, 2H), 7.66 (s, 1H), 7.55-7.28 (m, 5H), 7.19 (d, J=8.0 Hz, 1H), 7.12 (d, J=8.0 Hz, 1H), 7.00 (s, 1H), 5.25 (s, 2H), 4.57 (s, 2H), 4.23 (d, J=6.2 Hz, 2H), 3.90 (q, J=7.1 Hz, 2H), 3.60 (s, 2H), 3.33 (s, 3H), 1.39 (s, 9H), 0.95 (t, J=7.1 Hz, 3H).

### Step-6: Preparation of ethyl 2-(4-acetyl-2-((7-(3-(((tert-butoxycarbonyl)amino)methyl)phenyl)-2-(methoxymethyl)benzofuran-5-yl)methoxy)phenyl)acetate (12h)

To a solution of ethyl 2-(4-bromo-2-((7-(3-(((tert-butoxycarbonyl)amino)methyl)phenyl)-2-(methoxymethyl)benzofuran-5-yl)methoxy)phenyl)acetate (12g) (800 mg, 1.253 mmol) in toluene (15 mL) was added tributyl(1-ethoxyvinyl)stannane (0.558 mL, 1.566 mmol) and Pd(Ph₃P)₄ (145 mg, 0.125 mmol), the resulting mixture was stirred at 120° C. for 20 h under nitrogen. The reaction mixture was cooled to room temperature, diluted with EtOAc (150 mL) and water (100 mL), quenched with 3N aqueous HCl (1.253 mL, 3.76 mmol) and stirred for 10 min, filtered through a Celite pad. The organic layer was separated, and the aq. layer was extracted with additional ethyl acetate (100 mL). The combined organic layers were washed with brine (100 mL), dried, filtered and evaporated to dryness. The residue obtained was purified by flash column chromatography [silica gel (40 g), eluting with ethyl acetate in hexanes from 0-35%] to furnish ethyl 2-(4-acetyl-2-((7-(3-(((tert-butoxycarbonyl)amino)methyl)phenyl)-2-(methoxymethyl)benzofuran-5-yl)methoxy)phenyl)acetate (12h) (285 mg, 38% yield) as a clear oil. ¹H NMR (300 MHz, DMSO-d₆) δ 7.76-7.67 (m, 3H), 7.62 (s, 1H), 7.60-7.55 (m, 2H), 7.50 (d, J=7.7 Hz, 1H), 7.46 (d, J=5.9 Hz, 1H), 7.39 (d, J=7.7 Hz, 1H), 7.30 (d, J=7.6 Hz, 1H), 7.00 (s, 1H), 5.32 (s, 2H), 4.57 (s, 2H), 4.22 (d, J=6.1 Hz, 2H), 3.92 (q, J=7.1 Hz, 2H), 3.71 (s, 2H), 3.32 (s, 3H), 2.58 (s, 3H), 1.38 (s, 9H), 0.96 (t, J=7.1 Hz, 3H).

### Step-7: Preparation of ethyl 2-(4-acetyl-2-((7-(3-(aminomethyl)phenyl)-2-(methoxymethyl)benzofuran-5-yl)methoxy)phenyl)acetate (12i)

To a solution of ethyl 2-(4-acetyl-2-((7-(3-(((tert-butoxycarbonyl)amino)methyl)phenyl)-2-(methoxymethyl)benzofuran-5-yl)methoxy)phenyl)acetate (12h) (250 mg, 0.415 mmol) in DCM (10 mL) was added TFA (0.617 mL, 8.31 mmol) and stirred at RT for 18 h. The reaction mixture was concentrated to dryness to afford ethyl 2-(4-acetyl-2-((7-(3-(aminomethyl)phenyl)-2-(methoxymethyl)benzofuran-5-yl)methoxy)phenyl)acetate (12i) which was used as such in the next step without further purification; MS (ES+): 502.20 (M+1).

### Step-8: Preparation of 2-(4-acetyl-2-((7-(3-(aminomethyl)phenyl)-2-(methoxymethyl)benzofuran-5-yl)methoxy)phenyl)acetic acid (12j)

Compound 12j was prepared according to procedure reported in Step-8 of scheme 1 from ethyl 2-(4-acetyl-2-((7-(3-(aminomethyl)phenyl)-2-(methoxymethyl)benzofuran-5-yl)methoxy)phenyl)acetate (12i) (208 mg, 0.415 mmol) in THE (5 mL), methanol (5 mL) using a solution of lithium hydroxide monohydrate (142 mg, 3.32 mmol) in water (5 mL) and stirring for 16 h at room temperature. This gave after workup and purification by reverse-phase column chromatography [C-18 column, eluting with 0.1% aq HCl in water and acetonitrile from 0-100%]2-(4-acetyl-2-((7-(3-(aminomethyl)phenyl)-2-(methoxymethyl)benzofuran-5-yl)methoxy)phenyl)acetic acid (12j) (11 mg, 51% yield) HCl salt as a white solid; ¹H NMR (300 MHz, DMSO-d₆) δ 12.35 (s, 1H, D₂O exchangeable), 8.42 (s, 3H, D₂O exchangeable), 7.97-7.95 (m, 1H), 7.92 (dt, J=7.7, 1.6 Hz, 1H), 7.75 (d, J=1.6 Hz, 1H), 7.66 (d, J=1.7 Hz, 1H), 7.64-7.53 (m, 4H), 7.40 (d, J=7.5 Hz, 1H), 7.01 (s, 1H), 5.35 (s, 2H), 4.58 (s, 2H), 4.19-4.09 (m, 2H), 3.69 (s, 2H), 3.33 (s, 3H), 2.57 (s, 3H); MS (ES+): 474.1 (M+1); Analysis calculated for C₂₈H₂₇NO₆·HCl·H₂O: C, 63.69; H, 5.73; Cl, 6.71; N, 2.65; Found: C, 63.79; H, 5.60; Cl, 6.42; N, 2.79.

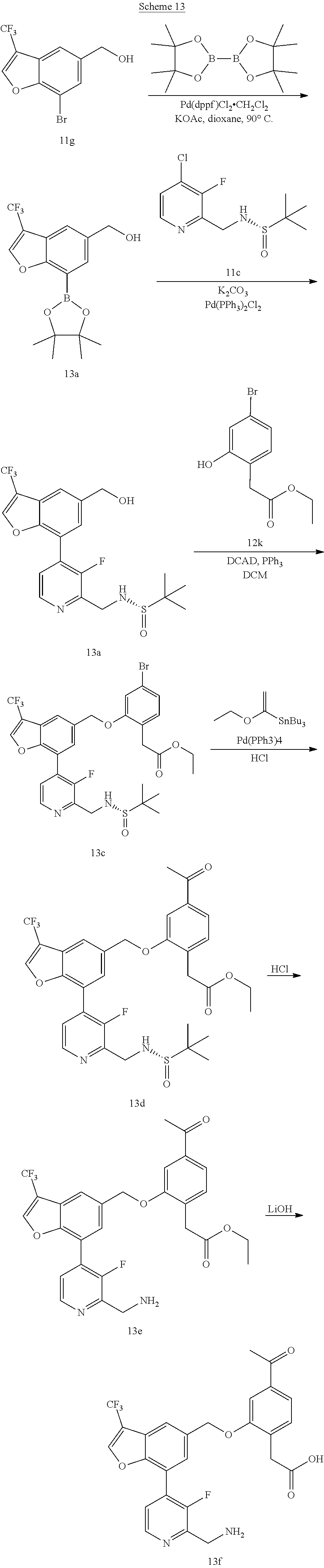

### Preparation of 2-(4-acetyl-2-((7-(2-(aminomethyl)-3-fluoropyridin-4-yl)-3-(trifluoromethyl)benzofuran-5-yl)methoxy)phenyl)acetic acid (13f)

### Step-1: Preparation of (7-(4,4,5,5-tetramethyl-1,3,2-dioxaborolan-2-yl)-3-(trifluoromethyl)benzofuran-5-yl)methanol (13a)

Compound 13a was prepared according to procedure reported in Step-3 of scheme 1 from (7-bromo-3-(trifluoromethyl)benzofuran-5-yl)methanol (11g) (3 g, 10.17 mmol) in anhydrous dioxane (60 mL) using bis(pinacolato)diboron (3.87 g, 15.25 mmol), potassium acetate (2.99 g, 30.5 mmol), Pd(dppf)Cl₂—CH₂Cl₂ (0.830 g, 1.017 mmol) and heating at 90° C. for 18 h under an argon atmosphere. This gave after workup and purification by flash column chromatography [silica (120 g), eluting with EtOAc in hexane from 0-40%](7-(4,4,5,5-tetramethyl-1,3,2-dioxaborolan-2-yl)-3-(trifluoromethyl)benzofuran-5-yl)methanol (13a) (2.4 g, 69% yield) as an off-white solid; ¹H NMR (300 MHz, DMSO-d₆) δ 8.81-8.76 (m, 1H), 7.76 (s, 1H), 7.73 (d, J=1.7 Hz, 1H), 5.40-5.31 (m, 1H), 4.63 (d, J=5.9 Hz, 2H), 1.34 (s, 12H); ¹⁹F NMR (282 MHz, DMSO-d₆) δ −58.01.

### Step-2: Preparation of N-((3-fluoro-4-(5-(hydroxymethyl)-3-(trifluoromethyl)benzofuran-7-yl)pyridin-2-yl)methyl)-2-methylpropane-2-sulfinamide (13b)

Compound 13b was prepared according to procedure reported in Step-4 of scheme 1 from (7-(4,4,5,5-tetramethyl-1,3,2-dioxaborolan-2-yl)-3-(trifluoromethyl)benzofuran-5-yl)methanol (13a) (1.8 g, 5.26 mmol) in dioxane (75 mL) using (+)-N-((4-chloro-3-fluoropyridin-2-yl)methyl)-2-methylpropane-2-sulfinamide (11c) (1.393 g, 5.26 mmol), bis(triphenylphosphine)Palladium(II)chloride [Pd(PPh₃)₂Cl₂](0.739 g, 1.052 mmol), a solution of K₂CO₃ (2.181 g, 15.78 mmol) in water (9 mL) and heating at 100° C. for 14 h on oil bath. This gave after workup and purification by flash column chromatography [silica gel (120 g), eluting with 0 to 75% 9:1 ethyl acetate/methanol in hexanes]N-((3-fluoro-4-(5-(hydroxymethyl)-3-(trifluoromethyl)benzofuran-7-yl)pyridin-2-yl)methyl)-2-methylpropane-2-sulfinamide (13b) (1.45 g, 62% yield); ¹H NMR (300 MHz, DMSO-d₆) δ 8.88-8.83 (m, 1H), 8.53 (d, J=4.9 Hz, 1H), 7.79 (s, 1H), 7.67 (t, J=5.2 Hz, 1H), 7.59 (s, 1H), 5.85 (t, J=5.9 Hz, 1H), 5.47 (t, J=5.7 Hz, 1H), 4.70 (d, J=5.8 Hz, 2H), 4.44-4.39 (m, 2H), 1.11 (s, 9H); ¹⁹F NMR (282 MHz, DMSO-d₆) δ −58.11, −128.31; MS (ES+): 445.10 (M+1).

### Step-3: Preparation of ethyl 2-(4-bromo-2-((7-(2-((1,1-dimethylethylsulfinamido)methyl)-3-fluoropyridin-4-yl)-3-(trifluoromethyl)benzofuran-5-yl)methoxy)phenyl)acetate (13c)

Compound 13c was prepared according to procedure reported in Step-2 of scheme 1 from N-((3-fluoro-4-(5-(hydroxymethyl)-3-(trifluoromethyl)benzofuran-7-yl)pyridin-2-yl)methyl)-2-methylpropane-2-sulfinamide (13b) (1.4 g, 3.15 mmol) in DCM (40 mL) using triphenylphosphine (1.074 g, 4.10 mmol), ethyl 2-(4-bromo-2-hydroxyphenyl)acetate (12k) (0.816 g, 3.15 mmol) and a solution of (E)-bis(4-chlorobenzyl) diazene-1,2-dicarboxylate (DCAD) (1.504 g, 4.10 mmol) in DCM (40 mL) and stirring at room temperature for 1 h. This gave after workup and purification by flash column chromatography (silica gel (120 g), eluting with 0 to 50% 9:1 ethyl acetate/methanol in hexanes) 2-(4-bromo-2-((7-(2-((1,1-dimethylethylsulfinamido)methyl)-3-fluoropyridin-4-yl)-3-(trifluoromethyl)benzofuran-5-yl)methoxy)phenyl)acetate (13c) (1.91 g, 88% yield) as a white semi-solid. ¹H NMR (300 MHz, DMSO-d₆) δ 8.92 (d, J=1.8 Hz, 1H), 8.56 (d, J=4.9 Hz, 1H), 7.94 (s, 1H), 7.74-7.52 (m, 2H), 7.36 (d, J=1.8 Hz, 1H), 7.24-7.09 (m, 2H), 5.86 (t, J=5.9 Hz, 1H), 5.35 (s, 2H), 4.46-4.38 (m, 2H), 3.89 (q, J=7.1 Hz, 2H), 3.61 (s, 2H), 1.11 (s, 9H), 0.95 (t, J=7.1 Hz, 3H); ¹⁹F NMR (282 MHz, DMSO-d₆) δ −58.08, −128.19.

### Step-4: Preparation of ethyl 2-(4-acetyl-2-((7-(2-((1,1-dimethylethylsulfinamido)methyl)-3-fluoropyridin-4-yl)-3-(trifluoromethyl)benzofuran-5-yl)methoxy)phenyl)acetate (13d)

Compound 13d was prepared according to procedure reported in Step-6 of scheme 12 from 2-(4-bromo-2-((7-(2-((1,1-dimethylethylsulfinamido)methyl)-3-fluoropyridin-4-yl)-3-(trifluoromethyl)benzofuran-5-yl)methoxy)phenyl)acetate (13c) (1.2 g, 1.750 mmol) in toluene (40 mL) using tributyl(1-ethoxyvinyl)stannane (0.780 mL, 2.188 mmol), Pd(Ph₃P)₄ (0.202 g, 0.175 mmol) and heating at 120° C. for 16 h under nitrogen. This gave after workup and purification by flash column chromatography (silica gel, 0 to 35% ethyl acetate in hexanes) ethyl 2-(4-acetyl-2-((7-(2-((1,1-dimethylethylsulfinamido)methyl)-3-fluoropyridin-4-yl)-3-(trifluoromethyl)benzofuran-5-yl)methoxy)phenyl)acetate (13d) (154 mg, 14% yield) as a clear oil. ¹H NMR (300 MHz, DMSO-d₆) δ 8.94-8.88 (m, 1H), 8.56 (d, J=4.9 Hz, 1H), 7.98 (s, 1H), 7.75 (s, 1H), 7.70 (t, J=5.2 Hz, 1H), 7.65-7.63 (m, 1H), 7.59 (dd, J=7.7, 1.5 Hz, 1H), 7.40 (d, J=7.7 Hz, 1H), 5.86 (t, J=5.8 Hz, 1H), 5.42 (s, 2H), 4.45-4.40 (m, 2H), 3.91 (q, J=7.1 Hz, 2H), 3.72 (s, 2H), 2.59 (s, 3H), 1.11 (s, 9H), 0.96 (t, J=7.1 Hz, 3H); ¹⁹F NMR (282 MHz, DMSO-d₆) δ −58.09, −128.13; MS (ES+): 649.20 (M+1).

### Step-5: Preparation of ethyl 2-(4-acetyl-2-((7-(2-(aminomethyl)-3-fluoropyridin-4-yl)-3-(trifluoromethyl)benzofuran-5-yl)methoxy)phenyl)acetate (13e)

To a solution of ethyl 2-(4-acetyl-2-((7-(2-((1,1-dimethylethylsulfinamido)methyl)-3-fluoropyridin-4-yl)-3-(trifluoromethyl)benzofuran-5-yl)methoxy)phenyl)acetate (13d) (140 mg, 0.216 mmol) in tetrahydrofuran (15 mL) was added aqueous 2N HCl (0.216 mL, 0.647 mmol) and stirred at RT for 4 h. The reaction mixture was concentrated to dryness to afford ethyl 2-(4-acetyl-2-((7-(2-(aminomethyl)-3-fluoropyridin-4-yl)-3-(trifluoromethyl)benzofuran-5-yl)methoxy)phenyl)acetate (13e) which was used as such in the next step without further purification; MS (ES+): 545.20 (M+1).

### Step-6: Preparation of 2-(4-acetyl-2-((7-(2-(aminomethyl)-3-fluoropyridin-4-yl)-3-(trifluoromethyl)benzofuran-5-yl)methoxy)phenyl)acetic acid (13f)

Compound 13f was prepared according to procedure reported in Step-8 of scheme 1 from ethyl 2-(4-acetyl-2-((7-(2-(aminomethyl)-3-fluoropyridin-4-yl)-3-(trifluoromethyl)benzofuran-5-yl)methoxy)phenyl)acetate (13e) (118 mg, 0.216 mmol) in THE (5 mL), methanol (5 mL) using a solution of lithium hydroxide monohydrate (74.0 mg, 1.728 mmol) in water (5 mL) and stirring for 16 h at room temperature. This gave after workup and purification by reverse-phase column chromatography [C-18 column, eluting with 0.1% aq HCl in water and acetonitrile from 0-100%]2-(4-acetyl-2-((7-(2-(aminomethyl)-3-fluoropyridin-4-yl)-3-(trifluoromethyl)benzofuran-5-yl)methoxy)phenyl)acetic acid (13f) (5.5 mg, 5% yield) HCl salt as a white solid; ¹H NMR (300 MHz, DMSO-d₆) δ 12.28 (s, 1H, D₂O exchangeable), 8.96-8.93 (m, 1H), 8.67 (d, J=5.0 Hz, 1H), 8.45 (s, 3H, D₂O exchangeable), 8.05 (s, 1H), 7.86-7.80 (m, 2H), 7.64-7.57 (m, 2H), 7.40 (d, J=7.7 Hz, 1H), 5.45 (s, 2H), 4.45-4.36 (m, 2H), 3.68 (s, 2H), 2.58 (s, 3H); ¹⁹F NMR (282 MHz, DMSO-d₆) δ −58.04, −128.43; MS (ES+): 517.1 (M+1).

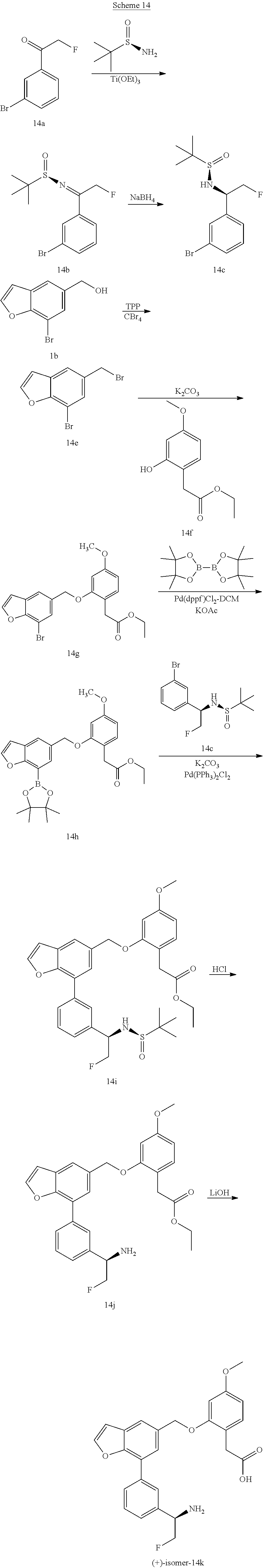

### Preparation of (+)-2-(2-((7-(3-(1-amino-2-fluoroethyl)phenyl)benzofuran-5-yl)methoxy)-4-methoxyphenyl)acetic acid (14k)

### Step-1: Preparation of (+)-N-(1-(3-bromophenyl)-2-fluoroethylidene)-2-methylpropane-2-sulfinamide (14b)

Compound 14b was prepared according to the procedure reported in step-5 of scheme 1 from 1-(3-bromophenyl)-2-fluoroethanone (14a) (3 g, 13.82 mmol; CAS #1219632-64-0) and (R)-2-methylpropane-2-sulfinamide (3.35 g, 27.6 mmol) in tetrahydrofuran (50 mL) using tetraethoxytitanium (6.31 g, 27.6 mmol). This gave after workup and purification by flash column chromatography [(silica gel (40 g), eluting with ethyl acetate in hexanes (0 to 20%)](+)-N-(1-(3-bromophenyl)-2-fluoroethylidene)-2-methylpropane-2-sulfinamide (14b) (2.08 g, 47% yield) as a brown syrup; MS (ES+): 320.0, 322.0 (M+1); Optical rotation [α]_{D}=+2.76 (c=1.02, MeOH)

### Step-2: Preparation of (−)-N-(1-(3-bromophenyl)-2-fluoroethyl)-2-methylpropane-2-sulfinamide (14c)

Compound 14c was prepared according to the procedure reported in step-3 of scheme 5 from (+)-N-(1-(3-bromophenyl)-2-fluoroethylidene)-2-methylpropane-2-sulfinamide (14b) (2.0 g, 6.25 mmol) in tetrahydrofuran (40 mL) and water (1 mL) using sodium borohydride (0.709 g, 18.74 mmol). This gave after workup and purification by flash column chromatography [(silica gel 80 g, eluting with ethyl acetate in hexanes (0 to 100%)](−)-N-(1-(3-bromophenyl)-2-fluoroethyl)-2-methylpropane-2-sulfinamide (14c) (1.5 g, 75% yield) as a white solid; ¹H NMR (300 MHz, DMSO-d₆) δ 7.71 (t, J=1.9 Hz, 1H), 7.56-7.41 (m, 2H), 7.33 (t, J=7.8 Hz, 1H), 6.05 (d, J=8.9 Hz, 1H), 4.65-4.50 (m, 2H), 4.42 (d, J=6.3 Hz, 1H), 1.13 (s, 9H); ¹⁹F NMR (282 MHz, DMSO-d₆) δ −217.83; Optical rotation [α]D=−7.16 (c=1.01, MeOH).

### Step-3: Preparation of 7-bromo-5-(bromomethyl)benzofuran (14e)

To a solution of (7-bromobenzofuran-5-yl)methanol (1b) (3.00 g, 13.21 mmol), CBr₄ (8.76 g, 26.4 mmol) in DCM (100 mL) was added at 0° C. a solution of triphenylphosphine (6.93 g, 26.4 mmol) in DCM (50 mL) over a period of 15 mins and stirred at 0° C. for 2 h. The reaction mixture diluted with water (200 mL) and extracted with DCM (2×150 mL). The combined organic layers were dried, filtered and evaporated to dryness. The residue obtained was purified by flash column chromatography [silica gel (40 g), eluting with ethyl acetate in hexanes from 0-100%] to furnish 7-bromo-5-(bromomethyl)benzofuran (14e) (2.38 g, 62% yield) as a pale yellow solid; ¹H NMR (300 MHz, DMSO-d₆) δ 8.16 (d, J=2.2 Hz, 1H), 7.78 (d, J=1.6 Hz, 1H), 7.68 (d, J=1.6 Hz, 1H), 7.12 (d, J=2.2 Hz, 1H), 4.84 (s, 2H).

### Step-4: Preparation of ethyl 2-(2-((7-bromobenzofuran-5-yl)methoxy)-4-methoxyphenyl)acetate (14g)

To a stirred solution of 7-bromo-5-(bromomethyl)benzofuran (14e) (1.379 g, 4.76 mmol) in DMF (10 mL) was added K₂CO₃ (1.972 g, 14.27 mmol) followed by ethyl 2-(2-hydroxy-4-methoxyphenyl)acetate (14f) (1 g, 4.76 mmol; CAS #76322-29-7), K₂CO₃ (1.972 g, 14.27 mmol). The resultant mixture was stirred at room temperature for 12 h and diluted with EtOAc (100 mL) and brine (100 mL). The organic layer was dried, filtered, evaporated to dryness to afford ethyl 2-(2-((7-bromobenzofuran-5-yl)methoxy)-4-methoxyphenyl)acetate (14g) (1.95 g, 98% yield) as white solid; ¹H NMR (300 MHz, DMSO-d₆) δ 8.14 (d, J=2.2 Hz, 1H), 7.71 (d, J=1.5 Hz, 1H), 7.59 (d, J=1.5 Hz, 1H), 7.17-7.04 (m, 2H), 6.66 (d, J=2.4 Hz, 1H), 6.49 (dd, J=8.3, 2.4 Hz, 1H), 5.17 (s, 2H), 4.01 (q, J=7.1 Hz, 2H), 3.74 (s, 3H), 3.53 (s, 2H), 1.07 (t, J=7.1 Hz, 3H).

### Step-5: Preparation of ethyl 2-(4-methoxy-2-((7-(4,4,5,5-tetramethyl-1,3,2-dioxaborolan-2-yl)benzofuran-5-yl)methoxy)phenyl)acetate (14h)

Compound 14h was prepared according to procedure reported in Step-3 of scheme 1 from ethyl 2-(2-((7-bromobenzofuran-5-yl)methoxy)-4-methoxyphenyl)acetate (14g) (4.00 g, 9.54 mmol) in anhydrous dioxane (100 mL) using bis(pinacolato)diboron (3.63 g, 14.31 mmol), potassium acetate (2.81 g, 28.6 mmol), Pd(dppf)Cl₂—CH₂Cl₂ (0.779 g, 0.954 mmol) and heating at 90° C. for 12 h under an argon atmosphere. This gave after workup and purification by flash column chromatography [silica (120 g), eluting with EtOAc in hexane from 0-15%]ethyl 2-(4-methoxy-2-((7-(4,4,5,5-tetramethyl-1,3,2-dioxaborolan-2-yl)benzofuran-5-yl)methoxy)phenyl)acetate (14h) (3.61 g, 81% yield) as a pale yellow oil; ¹H NMR (300 MHz, DMSO-d₆) δ 8.06 (d, J=2.2 Hz, 1H), 7.83 (d, J=1.9 Hz, 1H), 7.65 (d, J=1.9 Hz, 1H), 7.10 (d, J=8.3 Hz, 1H), 6.97 (d, J=2.3 Hz, 1H), 6.68 (d, J=2.4 Hz, 1H), 6.48 (dd, J=8.3, 2.4 Hz, 1H), 5.16 (s, 2H), 3.99 (q, J=7.1 Hz, 2H), 3.74 (s, 3H), 3.50 (s, 2H), 1.34 (s, 12H), 1.10-1.00 (m, 3H); MS (ES+): 489.20 (M+Na).

### Step-6: Preparation of ethyl 2-(2-((7-(3-(-1-((R)-1,1-dimethylethylsulfinamido)-2-fluoroethyl)phenyl)benzofuran-5-yl)methoxy)-4-methoxyphenyl)acetate (14i)

Compound 14i was prepared according to procedure reported in Step-4 of scheme 1 from ethyl 2-(4-methoxy-2-((7-(4,4,5,5-tetramethyl-1,3,2-dioxaborolan-2-yl)benzofuran-5-yl)methoxy)phenyl)acetate (14h) (1.20 g, 2.57 mmol) in dioxane (25 mL) using (−)-N-(1-(3-bromophenyl)-2-fluoroethyl)-2-methylpropane-2-sulfinamide (14c) (0.995 g, 3.09 mmol), bis(triphenylphosphine)Palladium(II)chloride [Pd(PPh₃)₂Cl₂](0.271 g, 0.386 mmol), a solution of K₂CO₃ (1.067 g, 7.72 mmol) in water (3 mL) and heating at 100° C. for 4 h on oil bath. This gave after workup and purification by flash column chromatography [silica gel (40 g), eluting with 0 to 50% ethyl acetate in hexanes]ethyl 2-(2-((7-(3-(1-((R)-1,1-dimethylethylsulfinamido)-2-fluoroethyl)phenyl)benzofuran-5-yl)methoxy)-4-methoxyphenyl)acetate (14i) (1.06 g, 71% yield) as a white solid; ¹H NMR (300 MHz, DMSO-d₆) δ 8.09 (d, J=2.2 Hz, 1H), 7.92 (s, 1H), 7.85-7.79 (m, 1H), 7.71 (d, J=1.6 Hz, 1H), 7.59 (d, J=1.6 Hz, 1H), 7.53 (d, J=4.7 Hz, 2H), 7.11 (d, J=8.3 Hz, 1H), 7.06 (d, J=2.2 Hz, 1H), 6.70 (d, J=2.4 Hz, 1H), 6.48 (dd, J=8.3, 2.4 Hz, 1H), 6.03 (d, J=8.2 Hz, 1H), 5.23 (s, 2H), 4.73-4.62 (m, 2H), 4.52 (d, J=6.3 Hz, 1H), 3.91 (q, J=7.1 Hz, 2H), 3.74 (s, 3H), 3.54 (s, 2H), 1.14 (s, 9H), 0.97 (t, J=7.1 Hz, 3H); ¹⁹F NMR (282 MHz, DMSO-d₆) δ −217.05; MS (ES+): 582.20 (M+1).

### Step-7: Preparation of ethyl 2-(2-((7-(3-(1-amino-2-fluoroethyl)phenyl)benzofuran-5-yl)methoxy)-4-methoxyphenyl)acetate (14j)

To a solution of ethyl 2-(2-((7-(3-(1-((R)-1,1-dimethylethylsulfinamido)-2-fluoroethyl)phenyl)benzofuran-5-yl)methoxy)-4-methoxyphenyl)acetate (14i) (1.00 g, 1.719 mmol) in tetrahydrofuran (40 mL) was added aqueous 3N HCl (1.719 mL, 5.16 mmol) and stirred at RT for 2 h. The reaction mixture was concentrated to dryness to afford ethyl 2-(2-((7-(3-(1-amino-2-fluoroethyl)phenyl)benzofuran-5-yl)methoxy)-4-methoxyphenyl)acetate (14j) which was used as such in the next step without further purification; MS (ES+): 478.20 (M+1).

### Step-8: Preparation of (+)-2-(2-((7-(3-(1-amino-2-fluoroethyl)phenyl)benzofuran-5-yl)methoxy)-4-methoxyphenyl)acetic acid (14k)

Compound 14k was prepared according to procedure reported in Step-8 of scheme 1 ethyl 2-(2-((7-(3-(1-amino-2-fluoroethyl)phenyl)benzofuran-5-yl)methoxy)-4-methoxyphenyl)acetate (14j) (821 mg, 1.719 mmol) in THF (10 mL), methanol (10 mL) using a solution of lithium hydroxide monohydrate (589 mg, 13.75 mmol) in water (10 mL) and stirring for 16 h at room temperature. This gave after workup and purification by reverse-phase column chromatography [C-18 column, eluting with 0.1% aq HCl in water and acetonitrile from 0-100%](+)-2-(2-((7-(3-(1-amino-2-fluoroethyl)phenyl)benzofuran-5-yl)methoxy)-4-methoxyphenyl)acetic acid (14k) (450 mg, 58% yield) HCl salt as a white solid; ¹H NMR (300 MHz, DMSO-d₆) δ 9.14 (s, 3H, D₂O exchangeable), 8.10 (d, J=2.2 Hz, 1H), 8.09-8.06 (m, 1H), 7.98 (dt, J=7.2, 1.7 Hz, 1H), 7.77 (d, J=1.6 Hz, 1H), 7.70-7.59 (m, 3H), 7.12 (d, J=8.3 Hz, 1H), 7.06 (d, J=2.2 Hz, 1H), 6.68 (d, J=2.4 Hz, 1H), 6.48 (dd, J=8.3, 2.3 Hz, 1H), 5.26 (s, 2H), 5.01-4.73 (m, 3H), 3.73 (s, 3H), 3.52 (s, 2H); ¹⁹F NMR (282 MHz, DMSO-d₆) δ −222.90; MS (ES+): 450.1 (M+1); (ES−): 448.15 (M−1); Analysis calculated for C₂₆H₂₄FNO₅·HCl·H₂O: C, 61.97; H, 5.40; Cl, 7.04; N, 2.78; Found: C, 62.08; H, 5.50; Cl, 6.85; N, 2.81; Optical rotation [α]_{D}=+17.692 (c=0.26, MeOH).

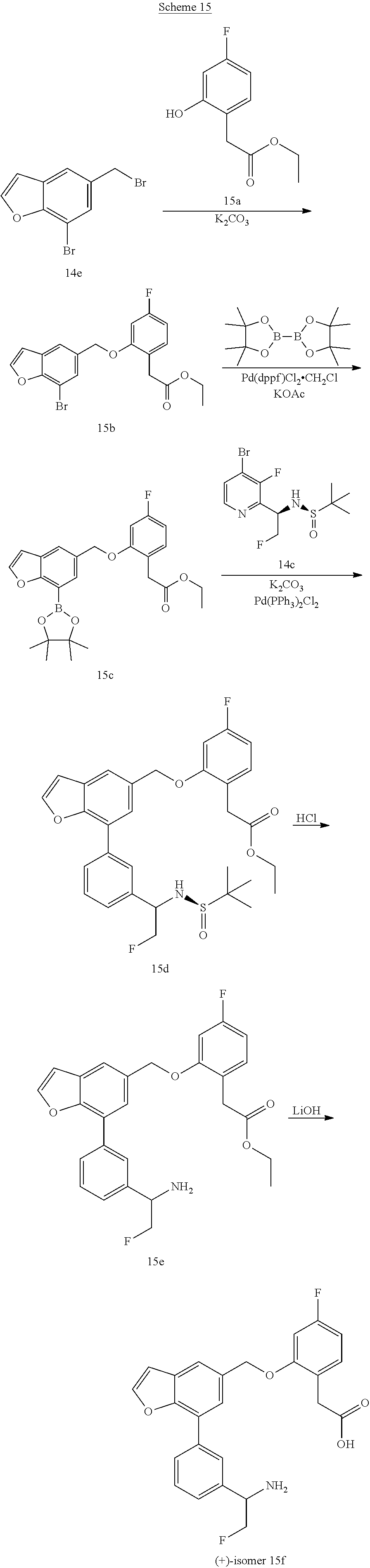

### Preparation of (+)-2-(2-((7-(3-(1-amino-2-fluoroethyl)phenyl)benzofuran-5-yl)methoxy)-4-fluorophenyl)acetic acid (15f)

### Step-1: Preparation of ethyl 2-(2-((7-bromobenzofuran-5-yl)methoxy)-4-fluorophenyl)acetate (15b)

Compound 15b was prepared according to the procedure reported in step-4 of scheme 14 from 7-bromo-5-(bromomethyl)benzofuran (14e) (2.63 g, 9.08 mmol) using ethyl 2-(4-fluoro-2-hydroxyphenyl)acetate (15a) (1.8 g, 9.08 mmol; CAS #1261751-44-3), K₂CO₃ (3.77 g, 27.2 mmol) in DMF (10 mL) and stirring at room temperature for 12h. This gave after workup and purification by flash column chromatography (SiO₂ (40 g), eluting with 0 to 50% EtOAc in hexane) ethyl 2-(2-((7-bromobenzofuran-5-yl)methoxy)-4-fluorophenyl)acetate (15b) (3.224 g, 87% yield) as a white solid. ¹H NMR (300 MHz, DMSO-d₆) δ 8.15 (d, J=2.2 Hz, 1H), 7.72 (d, J=1.5 Hz, 1H), 7.60 (d, J=1.5 Hz, 1H), 7.25 (dd, J=8.3, 6.9 Hz, 1H), 7.12 (d, J=2.2 Hz, 1H), 7.02 (dd, J=11.3, 2.5 Hz, 1H), 6.75 (td, J=8.5, 2.5 Hz, 1H), 5.20 (s, 2H), 4.01 (q, J=7.1 Hz, 2H), 3.60 (s, 2H), 1.07 (t, J=7.1 Hz, 3H); ¹⁹F NMR (282 MHz, DMSO-d₆) δ −112.61.

### Step-2: Preparation of ethyl 2-(4-fluoro-2-((7-(4,4,5,5-tetramethyl-1,3,2-dioxaborolan-2-yl)benzofuran-5-yl)methoxy)phenyl)acetate (15c)

Compound 15c was prepared according to the procedure reported in step-3 of scheme 1 from ethyl 2-(2-((7-bromobenzofuran-5-yl)methoxy)-4-fluorophenyl)acetate (15b) (1.5 g, 3.68 mmol), using bis(pinacolato)diboron (1.403 g, 5.53 mmol), potassium acetate (1.084 g, 11.05 mmol) and PdCl₂(dppf)-CH₂Cl₂ (0.301 g, 0.368 mmol) in anhydrous dioxane (50 mL) under an argon atmosphere and heating at 90° C. overnight. This gave after workup and purification by flash column chromatography [silica gel (40 g), eluting with EtOAc in hexanes from 0-40%]ethyl 2-(4-fluoro-2-((7-(4,4,5,5-tetramethyl-1,3,2-dioxaborolan-2-yl)benzofuran-5-yl)methoxy)phenyl)acetate (15c) (1.44 g, 86% yield) as a clear oil. ¹H NMR (300 MHz, DMSO-d₆) δ 8.07 (d, J=2.2 Hz, 1H), 7.83 (d, J=1.9 Hz, 1H), 7.65 (d, J=1.9 Hz, 1H), 7.24 (dd, J=8.4, 6.9 Hz, 1H), 7.03 (dd, J=11.4, 2.5 Hz, 1H), 6.97 (d, J=2.2 Hz, 1H), 6.74 (td, J=8.5, 2.5 Hz, 1H), 5.19 (s, 2H), 3.98 (q, J=7.1 Hz, 2H), 3.57 (d, J=1.7 Hz, 2H), 1.34 (s, 12H), 1.04 (t, J=7.1 Hz, 3H); ¹⁹F NMR (282 MHz, DMSO-d₆) δ −112.67.

### Step-3: Preparation of ethyl 2-(2-((7-(3-(1-((R)-1,1-dimethylethylsulfinamido)-2-fluoroethyl)phenyl)benzofuran-5-yl)methoxy)-4-fluorophenyl)acetate (15d)

Compound 15d was prepared according to procedure reported in Step-4 of scheme 1 from ethyl 2-(4-fluoro-2-((7-(4,4,5,5-tetramethyl-1,3,2-dioxaborolan-2-yl)benzofuran-5-yl)methoxy)phenyl)acetate (15c) (680 mg, 1.497 mmol) in dioxane (25 mL) using (−)-N-(1-(3-bromophenyl)-2-fluoroethyl)-2-methylpropane-2-sulfinamide (14c) (579 mg, 1.796 mmol), bis(triphenylphosphine)Palladium(II)chloride [Pd(PPh₃)₂Cl₂](158 mg, 0.225 mmol), a solution of K₂CO₃ (621 mg, 4.49 mmol) in water (3 mL) and heating at 100° C. for 4 h on oil bath. This gave after workup and purification by flash column chromatography [silica gel (40 g)]ethyl 2-(2-((7-(3-(1-((R)-1,1-dimethylethylsulfinamido)-2-fluoroethyl)phenyl)benzofuran-5-yl)methoxy)-4-fluorophenyl)acetate (15d) (553 mg, 65% yield); ¹H NMR (300 MHz, DMSO-d₆) δ 8.10 (d, J=2.2 Hz, 1H), 7.92 (s, 1H), 7.85-7.80 (m, 1H), 7.71 (s, 1H), 7.58 (s, 1H), 7.54 (s, 1H), 7.52 (s, 1H), 7.29-7.21 (m, 1H), 7.10-7.02 (m, 2H), 6.79-6.70 (m, 1H), 6.03 (d, J=8.3 Hz, 1H), 5.25 (s, 2H), 4.77-4.48 (m, 3H), 3.90 (q, J=7.1 Hz, 2H), 3.60 (s, 2H), 1.13 (s, 9H), 0.95 (t, J=7.1 Hz, 3H); MS (ES+): 570.20 (M+1).

### Step-4: Preparation of ethyl 2-(2-((7-(3-(1-amino-2-fluoroethyl)phenyl)benzofuran-5-yl)methoxy)-4-fluorophenyl)acetate (15e)

To a solution of ethyl 2-(2-((7-(3-(1-((R)-1,1-dimethylethylsulfinamido)-2-fluoroethyl)phenyl)benzofuran-5-yl)methoxy)-4-fluorophenyl)acetate (15d) (520 mg, 0.913 mmol) in tetrahydrofuran (20 mL) was added aqueous 3N HCl (0.913 mL, 2.74 mmol) and stirred at RT for 2 h. The reaction mixture was concentrated to dryness to afford ethyl 2-(2-((7-(3-(1-amino-2-fluoroethyl)phenyl)benzofuran-5-yl)methoxy)-4-fluorophenyl)acetate (15e) which was used as such in the next step without further purification; MS (ES+): 466.15 (M+1).

### Step-5: Preparation of (+)-2-(2-((7-(3-(1-amino-2-fluoroethyl)phenyl)benzofuran-5-yl)methoxy)-4-fluorophenyl)acetic acid (15f)

Compound 15f was prepared according to procedure reported in Step-8 of scheme 1 ethyl 2-(2-((7-(3-(1-amino-2-fluoroethyl)phenyl)benzofuran-5-yl)methoxy)-4-fluorophenyl)acetate (15e) (425 mg, 0.913 mmol) in THE (8 mL), methanol (8 mL) using a solution of lithium hydroxide monohydrate (313 mg, 7.30 mmol) in water (8 mL) and stirring for 16 h at room temperature. This gave after workup and purification by reverse-phase column chromatography [C-18 column, eluting with 0.1% aq HCl in water and acetonitrile from 0-100%](+)-2-(2-((7-(3-(1-amino-2-fluoroethyl)phenyl)benzofuran-5-yl)methoxy)-4-fluorophenyl)acetic acid (15f) (216 mg, 54% yield) HCl salt as a white solid; ¹H NMR (300 MHz, DMSO-d₆) δ 9.09 (s, 3H, D₂O exchangeable), 8.11 (d, J=2.2 Hz, 1H), 8.09-8.05 (m, 1H), 7.97 (dt, J=6.8, 1.9 Hz, 1H), 7.77 (d, J=1.6 Hz, 1H), 7.70-7.58 (m, 3H), 7.25 (dd, J=8.4, 6.9 Hz, 1H), 7.07 (d, J=2.2 Hz, 1H), 7.02 (dd, J=11.3, 2.5 Hz, 1H), 6.74 (td, J=8.5, 2.5 Hz, 1H), 5.28 (s, 2H), 5.00-4.71 (m, 3H), 3.57 (s, 2H); ¹⁹F NMR (282 MHz, DMSO-d₆) δ −112.95, −222.84; MS (ES+): 438.1 (M+1); (ES−): 436.1 (M−1); Analysis calculated for C₂₅H₂₁F₂NO₄·HCl·H₂O: C, 61.04; H, 4.92; Cl, 7.21; N, 2.85 Found: C, 60.93; H, 4.77; Cl, 7.21; N, 2.94; Optical rotation [α]_{D}=+13.89 (c=0.36, MeOH).

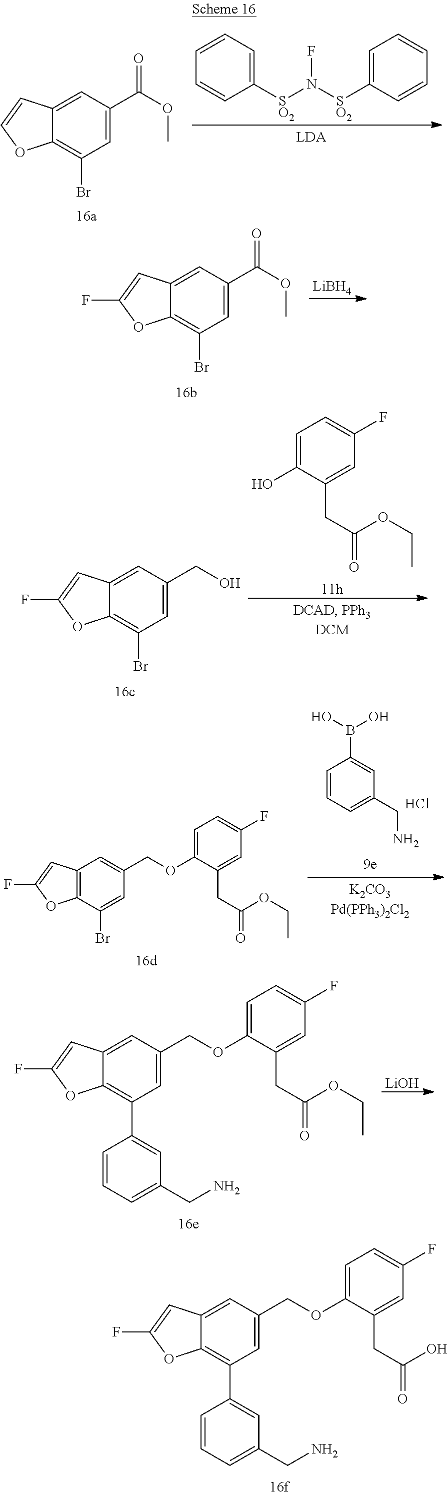

### Preparation of 2-(2-((7-(3-(aminomethyl)phenyl)-2-fluorobenzofuran-5-yl)methoxy)-5-fluorophenyl)acetic acid (16f)

### Step-1: Preparation of methyl 7-bromo-2-fluorobenzofuran-5-carboxylate (16b)

To a stirred solution of methyl 7-bromobenzofuran-5-carboxylate (16a) (5 g, 19.60 mmol; CAS #286836-79-1) in dry THF (90 mL) at −78° C. under N₂ was added dropwise LDA (19.6 mL, 1.5 M, 29.4 mmol). The mixture was kept at −78° C. for 1.5 h followed by the addition of a solution of N-fluoro-N-(phenylsulfonyl)benzenesulfonamide (12.5 g, 39.6 mmol) in THE (60 mL). The mixture was slowly warmed to room temperature and stirred overnight. The reaction was quenched with saturated NH₄Cl aqueous solution and extracted with ethyl acetate. The organic layers were combined, washed with brine, dried over Na₂SO₄ and concentrated. The residue was purified by flash column chromatography [silica (40 g), eluting with ethyl acetate/hexanes, 0-40%] to afford methyl 7-bromo-2-fluorobenzofuran-5-carboxylate (16b) (453 mg, 9% yield) as a yellow solid. ¹H NMR (300 MHz, DMSO-d₆) δ 8.24 (d, J=1.6 Hz, 1H), 8.06 (d, J=1.6 Hz, 1H), 6.64 (d, J=6.5 Hz, 1H), 3.89 (s, 3H); ¹⁹F NMR (282 MHz, DMSO-d₆) δ −109.09.

### Step-2: Preparation of (7-bromo-2-fluorobenzofuran-5-yl)methanol (16c)

Compound 16c was prepared according to the procedure reported in step-5 of scheme 11 from methyl 7-bromo-2-fluorobenzofuran-5-carboxylate (16b) (495 mg, 1.813 mmol) in THF (15 mL) using LiBH₄ (1.45 mL, 5.80 mmol) and MeOH (0.225 mL, 5.56 mmol). This gave after workup and purification by flash column chromatography [silica (12 g), eluting with EtOAc in hexane from 0-50%] followed by purification using reverse phase column chromatography [C18 (50 g), eluting with ACN in water (containing 0.1% HCl) from 0-100%](7-bromo-2-fluorobenzofuran-5-yl)methanol (16c) (90 mg, 20% yield) as a white solid; ¹H NMR (300 MHz, Chloroform-d) δ 7.46-7.25 (m, 2H), 5.89 (d, J=6.6 Hz, 1H), 4.65 (s, 2H), 2.84 (s, 1H); ¹⁹F NMR (282 MHz, Chloroform-d) δ −109.55.

### Step-3: Preparation of ethyl 2-(2-((7-bromo-2-fluorobenzofuran-5-yl)methoxy)-5-fluorophenyl)acetate (16d)

Compound 16d was prepared according to procedure reported in Step-2 of scheme 1 from (7-bromo-2-fluorobenzofuran-5-yl)methanol (16c) (1.5 g, 6.12 mmol) in DCM (60 mL) using triphenylphosphine (1.897 g, 7.23 mmol)) and ethyl 2-(5-fluoro-2-hydroxyphenyl)acetate (11h) (1.103 g, 5.56 mmol), a solution of bis(4-chlorobenzyl)azodicarboxylate (DCAD) (2.66 g, 7.23 mmol) in DCM (60 mL). This gave after workup and purification by flash column chromatography (silica gel (80 g), eluting with 0-20% hexanes/ethyl acetate) ethyl 2-(2-((7-bromo-2-fluorobenzofuran-5-yl)methoxy)-5-fluorophenyl)acetate (16d) (1.50 g, 63.4% yield) as a white solid. ¹H NMR (300 MHz, DMSO-d₆) δ 7.63 (d, J=1.5 Hz, 1H), 7.58 (d, J=1.5 Hz, 1H), 7.14 (dd, J=8.9, 2.6 Hz, 1H), 7.11-7.02 (m, 2H), 6.54 (d, J=6.4 Hz, 1H), 5.15 (s, 2H), 4.03 (q, J=7.1 Hz, 2H), 3.64 (s, 2H), 1.09 (t, J=7.1 Hz, 3H); ¹⁹F NMR (282 MHz, DMSO-d₆) δ −110.41, −123.85; MS (ES+): 446.90 (M+Na).

### Step-4: Preparation of ethyl 2-(2-((7-(3-(aminomethyl)phenyl)-2-fluorobenzofuran-5-yl)methoxy)-5-fluorophenyl)acetate (16e)

Compound 16e was prepared according to procedure reported in Step-4 of scheme 1 from ethyl 2-(2-((7-bromo-2-fluorobenzofuran-5-yl)methoxy)-5-fluorophenyl)acetate (16d) (200 mg, 0.470 mmol) in dioxane (20 mL) using 3-(aminomethyl)phenylboronic acid hydrochloride (9e) (132 mg, 0.706 mmol), bis(triphenylphosphine)Palladium(II)chloride [Pd(PPh₃)₂Cl₂](49.5 mg, 0.071 mmol), a solution of K₂CO₃ (195 mg, 1.411 mmol) in water (4 mL) and heating at 100° C. for 3 h on oil bath. This gave after workup and purification by flash column chromatography [silica gel (12 g), eluting with 0 to 5% MeOH in DCM]ethyl 2-(2-((7-(3-(aminomethyl)phenyl)-2-fluorobenzofuran-5-yl)methoxy)-5-fluorophenyl)acetate (16e) (158 mg, 74.4% yield); ¹H NMR (300 MHz, DMSO-d₆) δ 7.78-7.73 (m, 1H), 7.65 (dt, J=7.5, 1.7 Hz, 1H), 7.60 (d, J=1.6 Hz, 1H), 7.54 (d, J=1.7 Hz, 1H), 7.47 (t, J=7.5 Hz, 1H), 7.44-7.38 (m, 1H), 7.17-7.06 (m, 3H), 6.44 (d, J=6.4 Hz, 1H), 5.20 (s, 2H), 3.94 (q, J=7.1 Hz, 2H), 3.81 (s, 2H), 3.65 (s, 2H), 1.00 (t, J=7.1 Hz, 3H); MS (ES+): 452.10 (M+1).

### Step-5: Preparation of 2-(2-((7-(3-(aminomethyl)phenyl)-2-fluorobenzofuran-5-yl)methoxy)-5-fluorophenyl)acetic acid (16f)

Compound 16f was prepared according to procedure reported in Step-8 of scheme 1 from ethyl 2-(2-((7-(3-(aminomethyl)phenyl)-2-fluorobenzofuran-5-yl)methoxy)-5-fluorophenyl)acetate (16e) (150 mg, 0.332 mmol) in THE (5 mL), methanol (5 mL) using a solution of lithium hydroxide monohydrate (47.7 mg, 1.994 mmol) in water (5 mL) and stirring for 16 h at room temperature. This gave after workup and purification by reverse-phase column chromatography [C-18 column, eluting with 0.1% aq HCl in water and acetonitrile from 0-100%]2-(2-((7-(3-(aminomethyl)phenyl)-2-fluorobenzofuran-5-yl)methoxy)-5-fluorophenyl)acetic acid (16f) (104 mg, 74%) hydrochloride salt as a white solid; ¹H NMR (300 MHz, DMSO-d₆) δ 7.96-7.93 (m, 1H), 7.89-7.77 (m, 1H), 7.66 (d, J=1.6 Hz, 1H), 7.61 (d, J=1.7 Hz, 1H), 7.60-7.55 (m, 2H), 7.17-6.98 (m, 3H), 6.44 (d, J=6.4 Hz, 1H), 5.23 (s, 2H), 4.13 (s, 2H), 3.61 (s, 2H); ¹⁹F NMR (282 MHz, DMSO-d6) δ −111.52, −124.07; MS (ES+): 424.1 (M+1); Analysis calculated for C₂₄H₁₉F₂NO₄·HCl·H₂O: C, 60.32; H, 4.64; Cl, 7.42; N, 2.93; Found: C, 60.46; H, 4.80; Cl, 7.17; N, 2.96.

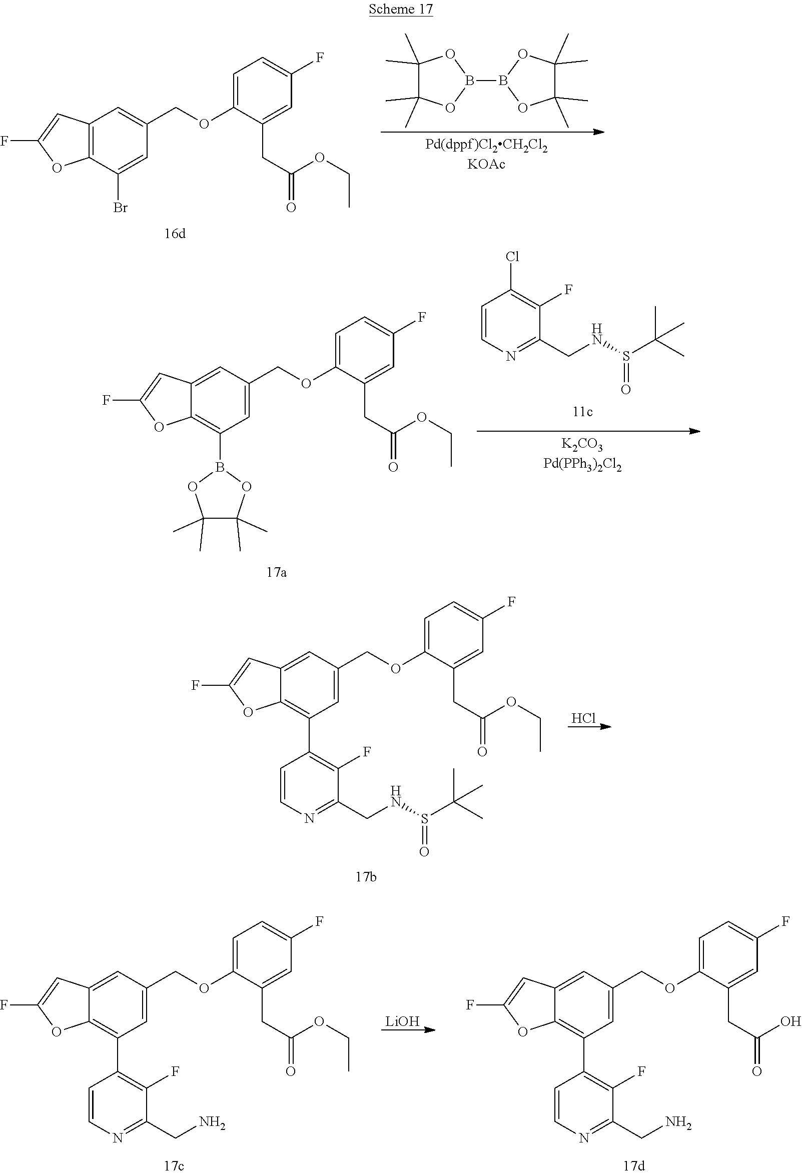

### Preparation of 2-(2-((7-(2-(aminomethyl)-3-fluoropyridin-4-yl)-2-fluorobenzofuran-5-yl)methoxy)-5-fluorophenyl)acetic acid (17d)

### Step-1: Preparation of ethyl 2-(5-fluoro-2-((2-fluoro-7-(4,4,5,5-tetramethyl-1,3,2-dioxaborolan-2-yl)benzofuran-5-yl)methoxy)phenyl)acetate (17a)

Compound 17a was prepared according to the procedure reported in step-3 of scheme 1 from ethyl 2-(2-((7-bromo-2-fluorobenzofuran-5-yl)methoxy)-5-fluorophenyl)acetate (16d) (1.04 g, 2.446 mmol), using bis(pinacolato)diboron (0.932 g, 3.67 mmol), potassium acetate (0.720 g, 7.34 mmol), PdCl₂(dppf)-CH₂Cl₂ (0.200 g, 0.245 mmol) in anhydrous dioxane (30 mL) under an argon atmosphere and heating at 90° C. for 18h. This gave after workup and purification by flash column chromatography [silica gel (40 g), eluting with EtOAc in hexanes from 0-20%]ethyl 2-(5-fluoro-2-((2-fluoro-7-(4,4,5,5-tetramethyl-1,3,2-dioxaborolan-2-yl)benzofuran-5-yl)methoxy)phenyl)acetate (17a) (954 mg, 83% yield). ¹H NMR (300 MHz, DMSO-d₆) δ 7.74 (d, J=1.9 Hz, 1H), 7.60 (d, J=1.9 Hz, 1H), 7.20-7.00 (m, 3H), 6.35 (d, J=6.4 Hz, 1H), 5.13 (s, 2H), 4.10-3.96 (m, 2H), 3.61 (s, 2H), 1.33 (s, 12H), 1.06 (t, J=7.1 Hz, 3H); MS (ES+): 495.15 (M+Na).

### Step-2: Preparation of ethyl 2-(2-((7-(2-((1,1-dimethylethylsulfinamido)methyl)-3-fluoropyridin-4-yl)-2-fluorobenzofuran-5-yl)methoxy)-5-fluorophenyl)acetate (17b)

Compound 17b was prepared according to procedure reported in Step-4 of scheme 1 from ethyl 2-(5-fluoro-2-((2-fluoro-7-(4,4,5,5-tetramethyl-1,3,2-dioxaborolan-2-yl)benzofuran-5-yl)methoxy)phenyl)acetate (17a) (450 mg, 0.953 mmol) in dioxane (25 mL) using (+)-N-((4-chloro-3-fluoropyridin-2-yl)methyl)-2-methylpropane-2-sulfinamide (11c) (303 mg, 1.143 mmol), bis(triphenylphosphine)Palladium(II)chloride [Pd(PPh₃)₂Cl₂](100 mg, 0.143 mmol), a solution of K₂CO₃ (395 mg, 2.86 mmol) in water (3 mL) and heating at 100° C. for 3 h on oil bath. This gave after workup and purification by flash column chromatography [silica gel (40 g), eluting with 0 to 2.5% methanol in DCM]ethyl 2-(2-((7-(2-((1,1-dimethylethylsulfinamido)methyl)-3-fluoropyridin-4-yl)-2-fluorobenzofuran-5-yl)methoxy)-5-fluorophenyl)acetate (17b) (239 mg, 0.416 mmol, 43.7% yield); MS (ES+): 575.1 (M+1).

### Step-3: Preparation of ethyl 2-(2-((7-(2-(aminomethyl)-3-fluoropyridin-4-yl)-2-fluorobenzofuran-5-yl)methoxy)-5-fluorophenyl)acetate (17c)

To a solution of ethyl 2-(2-((7-(2-((1,1-dimethylethylsulfinamido)methyl)-3-fluoropyridin-4-yl)-2-fluorobenzofuran-5-yl)methoxy)-5-fluorophenyl)acetate (17b) (220 mg, 0.383 mmol) in tetrahydrofuran (20 mL) was added aqueous 3N HCl (0.383 mL, 1.149 mmol) and stirred at RT for 2 h. The reaction mixture was concentrated to dryness to afford ethyl 2-(2-((7-(2-(aminomethyl)-3-fluoropyridin-4-yl)-2-fluorobenzofuran-5-yl)methoxy)-5-fluorophenyl)acetate (17c) which was used as such in the next step without further purification; MS (ES+): 471.15 (M+1).

### Step-4: Preparation of 2-(2-((7-(2-(aminomethyl)-3-fluoropyridin-4-yl)-2-fluorobenzofuran-5-yl)methoxy)-5-fluorophenyl)acetic acid (17d)

Compound 17d was prepared according to procedure reported in Step-8 of scheme 1 from ethyl 2-(2-((7-(2-(aminomethyl)-3-fluoropyridin-4-yl)-2-fluorobenzofuran-5-yl)methoxy)-5-fluorophenyl)acetate (17c) (180 mg, 0.383 mmol) in THE (2 mL), acetonitrile (1 mL) using a solution of 1 N lithium hydroxide monohydrate (1.149 mL, 1.149 mmol) and stirring for 24 h at room temperature. This gave after workup and purification by reverse-phase column chromatography [C-18 column, eluting with 0.1% aq HCl in water and acetonitrile from 0-100%]2-(2-((7-(2-(aminomethyl)-3-fluoropyridin-4-yl)-2-fluorobenzofuran-5-yl)methoxy)-5-fluorophenyl)acetic acid (17d) (7 mg, 4% yield) HCl salt as a white solid; ¹H NMR (300 MHz, DMSO-d₆) δ 12.32 (s, 1H, D₂O exchangeable), 8.64 (d, J=5.0 Hz, 1H), 8.48-8.38 (m, 3H, D₂O exchangeable), 7.82 (d, J=1.6 Hz, 1H), 7.80 (t, J=5.4 Hz, 1H), 7.57 (s, 1H), 7.17-7.03 (m, 3H), 6.51 (d, J=6.4 Hz, 1H), 5.25 (s, 2H), 4.49-4.29 (m, 2H), 3.60 (s, 2H); ¹⁹F NMR (282 MHz, DMSO-d₆) δ −111.15, −123.93, −128.48; MS (ES+): 443.1 (M+1).

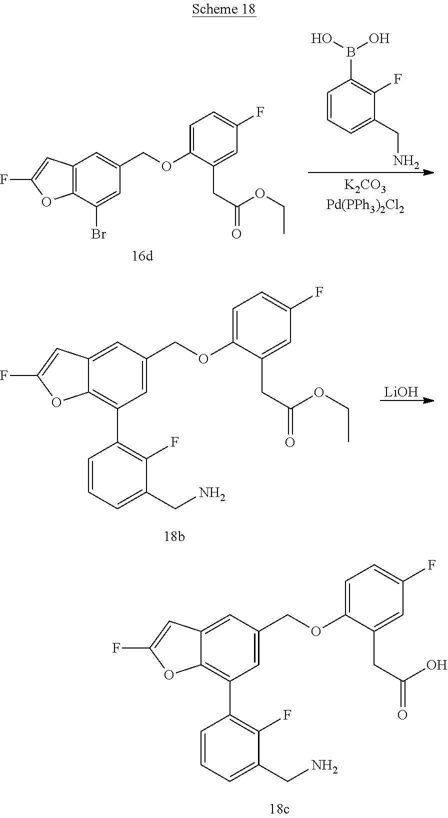

### Preparation of 2-(2-((7-(3-(aminomethyl)-2-fluorophenyl)-2-fluorobenzofuran-5-yl)methoxy)-5-fluorophenyl)acetic acid (18c)

### Step-1: Preparation of ethyl 2-(2-((7-(3-(aminomethyl)-2-fluorophenyl)-2-fluorobenzofuran-5-yl)methoxy)-5-fluorophenyl)acetate (18b)

Compound 18b was prepared according to procedure reported in Step-4 of scheme 1 from ethyl 2-(2-((7-bromo-2-fluorobenzofuran-5-yl)methoxy)-5-fluorophenyl)acetate (16d) (200 mg, 0.470 mmol) in dioxane (20 mL) using (3-(aminomethyl)-2-fluorophenyl)boronic acid hydrochloride (18a) (119 mg, 0.706 mmol), bis(triphenylphosphine)Palladium(II)chloride [Pd(PPh₃)₂Cl₂](49.5 mg, 0.071 mmol), a solution of K₂CO₃ (195 mg, 1.411 mmol) in water (4 mL) and heating at 100° C. for 3 h on oil bath. This gave after workup and purification by flash column chromatography [silica gel (12 g), eluting with 0 to 5% MeOH in DCM]ethyl 2-(2-((7-(3-(aminomethyl)-2-fluorophenyl)-2-fluorobenzofuran-5-yl)methoxy)-5-fluorophenyl)acetate (18b) (152 mg, 69% yield); ¹H NMR (300 MHz, DMSO-d₆) δ 7.66 (d, J=1.6 Hz, 1H), 7.65-7.58 (m, 1H), 7.49-7.42 (m, 1H), 7.39 (s, 1H), 7.32 (t, J=7.6 Hz, 1H), 7.18-7.05 (m, 3H), 6.45 (d, J=6.4 Hz, 1H), 5.19 (s, 2H), 3.92 (q, J=7.1 Hz, 2H), 3.84 (s, 2H), 3.63 (s, 2H), 0.99 (t, J=7.1 Hz, 3H); MS (ES+): 470.1 (M+1).

### Step-2: Preparation of 2-(2-((7-(3-(aminomethyl)-2-fluorophenyl)-2-fluorobenzofuran-5-yl)methoxy)-5-fluorophenyl)acetic acid (18c)

Compound 18c was prepared according to procedure reported in Step-8 of scheme 1 from ethyl 2-(2-((7-(3-(aminomethyl)-2-fluorophenyl)-2-fluorobenzofuran-5-yl)methoxy)-5-fluorophenyl)acetate (18b) (140 mg, 0.298 mmol) in THE (2 mL), acetonitrile (2 mL) using a solution of lithium hydroxide monohydrate (21.43 mg, 0.895 mmol) in water (1 mL) and stirring for 14 h at room temperature. This gave after workup and purification by reverse-phase column chromatography [C-18 column, eluting with 0.1% aq HCl in water and acetonitrile from 0-100%]2-(2-((7-(3-(aminomethyl)-2-fluorophenyl)-2-fluorobenzofuran-5-yl)methoxy)-5-fluorophenyl)acetic acid (18c) (54 mg, 41%) hydrochloride salt as a white solid; ¹H NMR (300 MHz, DMSO-d₆) δ 8.44 (s, 3H), 7.75-7.62 (m, 3H), 7.50-7.38 (m, 2H), 7.16-7.02 (m, 3H), 6.45 (d, J=6.4 Hz, 1H), 5.22 (s, 2H), 4.18 (s, 2H), 3.59 (s, 2H); ¹⁹F NMR (282 MHz, DMSO-d₆) δ −111.54, −118.50, −124.00; MS (ES+): 442.1 (M+1); Analysis calculated for C₂₄H₁₈F₃NO₄·HCl·H₂O: C, 58.13; H, 4.27; Cl, 7.15; N, 2.82; Found: C, 57.76; H, 4.22; Cl, 6.95; N, 2.99.

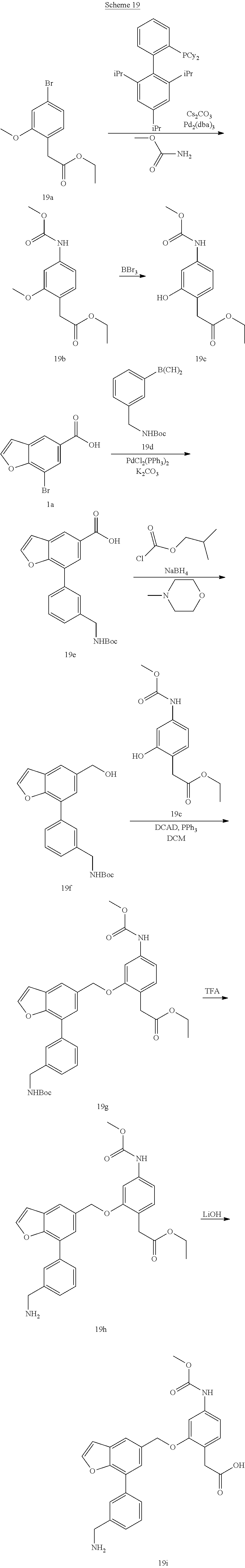

### Preparation of 2-(2-((7-(3-(aminomethyl)phenyl)benzofuran-5-yl)methoxy)-4-((methoxycarbonyl)amino)phenyl)acetic acid (19i)

### Step-1: Preparation of ethyl 2-(2-methoxy-4-(methoxycarbonylamino)phenyl)acetate (19b)

To a degassed solution of ethyl 2-(4-bromo-2-methoxyphenyl)acetate (19a) (750 mg, 2.75 mmol; CAS #1261570-38-0) in toluene (30 mL) was added dicyclohexyl(2′,4′,6′-triisopropylbiphenyl-2-yl)phosphine (131 mg, 0.275 mmol), methyl carbamate (309 mg, 4.12 mmol), Pd₂(dba)₃ (126 mg, 0.137 mmol), cesium carbonate (895 mg, 2.75 mmol) and heated under nitrogen at 90° C. for 19 h. The reaction mixture was diluted with ethyl acetate (100 mL), washed with water (60 mL), brine (60 mL), dried, filtered and concentrated in vacuum. The residue obtained was purified by flash column chromatography (silica gel (24g), eluting with 0 to 25% ethyl acetate in hexanes) to give ethyl 2-(2-methoxy-4-(methoxycarbonylamino)phenyl)acetate (19b) (452 mg, 61.6% yield) as a yellow solid; ¹H NMR (300 MHz, DMSO-d₆) δ 9.62 (s, 1H), 7.20-7.17 (m, 1H), 7.05 (d, J=8.1 Hz, 1H), 6.94 (dd, J=8.1, 2.0 Hz, 1H), 4.04 (q, J=7.1 Hz, 2H), 3.70 (s, 3H), 3.66 (s, 3H), 3.49 (s, 2H), 1.16 (t, J=7.1 Hz, 3H); MS (ES+): 268.1 (M+1)

### Step-2: Preparation of ethyl 2-(2-hydroxy-4-((methoxycarbonyl)amino)phenyl)acetate (19c)

To a solution of ethyl 2-(2-methoxy-4-(methoxycarbonylamino)phenyl)acetate (19b) (200 mg, 0.748 mmol) in DCM (15 mL) was cooled to −78° C. was added boron tribromide (0.283 mL, 2.99 mmol) and allowed to warm to room temperature slowly over a period of 14 h. The reaction mixture was cooled to 0° C., quenched with ethanol (10 mL) and concentrated to dryness. The mixture was reconstituted in ethanol (10 mL) and concentrated to dryness. The residue was taken in ethyl acetate and water. The organic layer was separated, washed with brine, dried, filtered, and concentrated in vacuum. The residue obtained was purified by flash column chromatography (silica gel (40 g), eluting with 0 to 25% ethyl acetate in hexanes) to give ethyl 2-(2-hydroxy-4-((methoxycarbonyl)amino)phenyl)acetate (19c) (47 mg, 24.80% yield) as a white solid; ¹H NMR (300 MHz, DMSO-d₆) δ 9.51-9.47 (m, 2H), 7.07 (d, J=2.1 Hz, 1H), 6.95 (d, J=8.2 Hz, 1H), 6.77 (dd, J=8.1, 2.0 Hz, 1H), 4.04 (q, J=7.1 Hz, 2H), 3.64 (s, 3H), 3.44 (s, 2H), 1.16 (t, J=7.1 Hz, 3H); MS (ES+): 254.00 (M+1)

### Step-3: Preparation of 7-(3-(((tert-butoxycarbonyl)amino)methyl)phenyl)benzofuran-5-carboxylic acid (19e)

Compound 19e was prepared according to the procedure reported in step-4 of scheme 1 from 7-bromobenzofuran-5-carboxylic acid (1a) (3 g, 12.45 mmol) in dioxane (100 mL) using (3-(((tert-butoxycarbonyl)amino)methyl)phenyl)boronic acid (19d) (4.38 g, 17.42 mmol), a solution of potassium bicarbonate (5.16 g, 37.3 mmol) in water (10 mL) and bis(triphenylphosphine)palladium(II) chloride (1.310 g, 1.867 mmol) and heating under a nitrogen atmosphere at 100° C. for 3 h on an oil bath. This gave after workup, purification by flash column chromatography [silica (40 g), eluting with DMA80 in DCM from 0-50%]7-(3-(((tert-butoxycarbonyl)amino)methyl)phenyl)benzofuran-5-carboxylic acid (19e) (2.7 g, 7.35 mmol, 59.0% yield) as a clear oil; ¹H NMR (300 MHz, DMSO-d₆) δ 8.22 (d, J=1.5 Hz, 1H), 8.12-8.05 (m, 2H), 7.76-7.68 (m, 2H), 7.56-7.43 (m, 2H), 7.29 (d, J=7.6 Hz, 1H), 7.10 (d, J=2.2 Hz, 1H), 4.23 (d, J=6.2 Hz, 2H), 1.39 (s, 9H); MS (ES−) 366.3 (M−1).

### Step-4: Preparation of tert-butyl 3-(5-(hydroxymethyl)benzofuran-7-yl)benzylcarbamate (19f)

Compound 19f was prepared according to the procedure reported in step-1 of scheme 1 from 7-(3-(((tert-butoxycarbonyl)amino)methyl)phenyl)benzofuran-5-carboxylic acid (19e) (1.7 g, 4.63 mmol) using N-methylmorpholine (0.610 mL, 5.55 mmol) in THE (40 mL), isobutyl chloroformate (0.729 mL, 5.55 mmol) and NaBH₄ (0.525 g, 13.88 mmol) in water (5 mL). This gave after workup tert-butyl 3-(5-(hydroxymethyl)benzofuran-7-yl)benzylcarbamate (19f) (1.1 g, 67.3% yield) as a white solid; ¹H NMR (300 MHz, DMSO-d₆) δ 8.02 (d, J=2.2 Hz, 1H), 7.73 (m, 2H), 7.59 (d, J=1.6 Hz, 1H), 7.52-7.38 (m, 3H), 7.28 (d, J=7.6 Hz, 1H), 7.01 (d, J=2.1 Hz, 1H), 5.23 (t, J=5.7 Hz, 1H), 4.64 (d, J=5.7 Hz, 2H), 4.22 (d, J=6.2 Hz, 2H), 1.40 (s, 9H).

### Step-5: Preparation of ethyl 2-(2-((7-(3-(((tert-butoxycarbonyl)amino)methyl)phenyl)benzofuran-5-yl)methoxy)-4-((methoxycarbonyl)amino)phenyl)acetate (19g)

Compound 19g was prepared according to procedure reported in Step-2 of scheme 1 from tert-butyl 3-(5-(hydroxymethyl)benzofuran-7-yl)benzylcarbamate (19f) (55.8 mg, 0.158 mmol) in DCM (5 mL) using triphenylphosphine (49.7 mg, 0.190 mmol) and ethyl 2-(2-hydroxy-4-((methoxycarbonyl)amino)phenyl)acetate (19c) (40 mg, 0.158 mmol), a solution of bis(4-chlorobenzyl)azodicarboxylate (DCAD) (69.6 mg, 0.190 mmol) in DCM (5 mL). This gave after workup and purification by flash column chromatography (silica gel (4g), ethyl 2-(2-((7-(3-(((tert-butoxycarbonyl)amino)methyl)phenyl)benzofuran-5-yl)methoxy)-4-((methoxycarbonyl)amino)phenyl)acetate (19g) (88 mg, 95% yield); MS (ES+): 611.10 (M+Na).

### Step-6: Preparation of ethyl 2-(2-((7-(3-(aminomethyl)phenyl)benzofuran-5-yl)methoxy)-4-((methoxycarbonyl)amino)phenyl)acetate (19h)

To a solution of ethyl 2-(2-((7-(3-(((tert-butoxycarbonyl)amino)methyl)phenyl)benzofuran-5-yl)methoxy)-4-((methoxycarbonyl)amino)phenyl)acetate (19g) (80 mg, 0.136 mmol) in DCM (10 mL) was added TFA (0.209 mL, 2.72 mmol) and stirred at RT for 16 h. The reaction mixture was concentrated to dryness to afford ethyl 2-(2-((7-(3-(aminomethyl)phenyl)benzofuran-5-yl)methoxy)-4-((methoxycarbonyl)amino)phenyl)acetate (19h) which was used as such in the next step without further purification; MS (ES+): 489.1 (M+1).

### Step-7: Preparation of 2-(2-((7-(3-(aminomethyl)phenyl)benzofuran-5-yl)methoxy)-4-((methoxycarbonyl)amino)phenyl)acetic acid (19i)

Compound 19i was prepared according to procedure reported in Step-8 of scheme 1 from ethyl 2-(2-((7-(3-(aminomethyl)phenyl)benzofuran-5-yl)methoxy)-4-((methoxycarbonyl)amino)phenyl)acetate (19h) (66.4 mg, 0.136 mmol) in THE (5 mL), methanol (5 mL) using a solution of lithium hydroxide monohydrate (34.9 mg, 0.816 mmol) in water (5 mL) and stirring for 16 h at room temperature. This gave after workup and purification by reverse-phase column chromatography [C-18 column, eluting with 0.1% aq HCl in water and acetonitrile from 0-100%]2-(2-((7-(3-(aminomethyl)phenyl)benzofuran-5-yl)methoxy)-4-((methoxycarbonyl)amino)phenyl)acetic acid (19i) (3 mg, 5% yield) HCl salt as a white solid; ¹H NMR (300 MHz, DMSO-d₆) δ 9.65 (s, 1H, D₂O exchangeable), 8.21 (s, 3H, D₂O exchangeable), 8.11 (d, J=2.2 Hz, 1H), 7.99 (s, 1H), 7.97-7.92 (m, 1H), 7.77 (s, 1H), 7.67-7.49 (m, 3H), 7.41-7.30 (m, 2H), 7.13-7.03 (m, 2H), 5.20 (s, 2H), 4.23-4.05 (m, 2H), 3.65 (s, 3H), 3.51 (s, 2H); MS (ES+): 461.1 (M+1); (ES−): 459.05 (M−1).

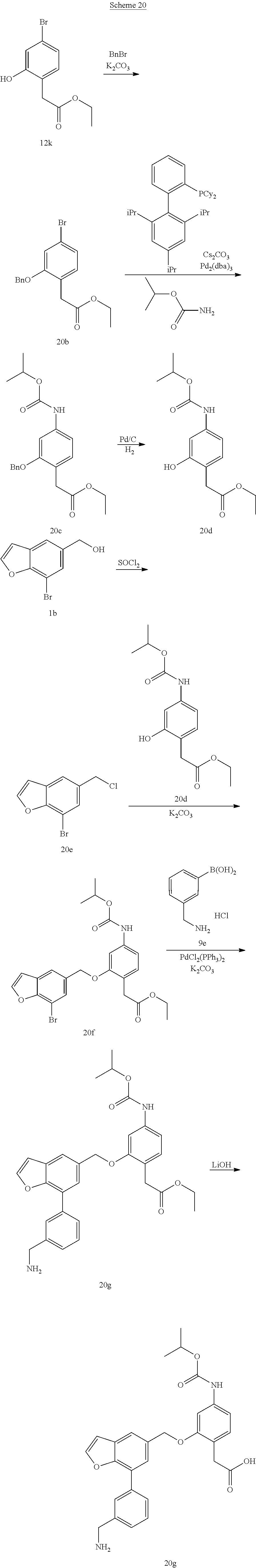

### Preparation of 2-(2-((7-(3-(aminomethyl)phenyl)benzofuran-5-yl)methoxy)-4-((isopropoxycarbonyl)amino)phenyl)acetic acid (20h)

### Step-1: Preparation of ethyl 2-(2-(benzyloxy)-4-bromophenyl)acetate (20b)

To a solution of ethyl 2-(4-bromo-2-hydroxyphenyl)acetate (12k) (8.8 g, 34 mmol; CAS #1261585-89-0) and (bromomethyl)benzene (5.81 g, 34.0 mmol) in DMF (100 mL) was added potassium carbonate (14.08 g, 102 mmol) and stirred at room temperature for 16 h. The reaction mixture was diluted with ethyl acetate (150 mL), washed with water (100 mL), brine (100 mL), dried, filtered, and concentrated in vacuum. The residue obtained was purified by flash column chromatography (silica gel (220 g), eluting with 0 to 8% ethyl acetate in hexanes) to give ethyl 2-(2-(benzyloxy)-4-bromophenyl)acetate (20b) (10.92 g, 92% yield); ¹H NMR (300 MHz, DMSO-d₆) δ 7.43-7.29 (m, 5H), 7.27 (d, J=1.8 Hz, 1H), 7.19 (d, J=8.0 Hz, 1H), 7.11 (dd, J=8.0, 1.8 Hz, 1H), 5.13 (s, 2H), 4.00 (q, J=7.1 Hz, 2H), 3.60 (s, 2H), 1.09 (t, J=7.1 Hz, 3H); MS (ES+): 348.95 (M+1).

### Step-2: Preparation of ethyl 2-(2-(benzyloxy)-4-((isopropoxycarbonyl)amino)phenyl)acetate (20c)

Compound 20c was prepared according to the procedure reported in step-1 of scheme 19 from ethyl 2-(2-(benzyloxy)-4-bromophenyl)acetate (20b) (750 mg, 2.148 mmol) in toluene (50 mL) using dicyclohexyl(2′,4′,6′-triisopropylbiphenyl-2-yl)phosphine (102 mg, 0.215 mmol), isopropyl carbamate (339 mg, 3.22 mmol), Pd₂(dba)₃ (98 mg, 0.107 mmol), cesium carbonate (700 mg, 2.148 mmol) and heated under nitrogen at 90° C. for 19 h. This gave after workup, purification by flash column chromatography [silica (40 g), eluting with 0 to 15% ethyl acetate in hexanes]ethyl 2-(2-(benzyloxy)-4-((isopropoxycarbonyl)amino)phenyl)acetate (20c) (428 mg, 54% yield); ¹H NMR (300 MHz, DMSO-d₆) δ 9.54 (s, 1H), 7.46-7.25 (m, 6H), 7.08 (d, J=8.2 Hz, 1H), 6.95 (dd, J=8.1, 1.9 Hz, 1H), 5.01 (s, 2H), 4.95-4.79 (m, 1H), 4.00 (q, J=7.1 Hz, 2H), 3.53 (s, 2H), 1.24 (d, J=6.2 Hz, 6H), 1.10 (t, J=7.1 Hz, 3H); MS (ES+): 372.10 (M+1).

### Step-3: Preparation of ethyl 2-(2-hydroxy-4-((isopropoxycarbonyl)amino)phenyl)acetate (20d)

To a solution of ethyl 2-(2-(benzyloxy)-4-((isopropoxycarbonyl)amino)phenyl)acetate (20c) (400 mg, 1.077 mmol) in ethyl acetate (20 mL) was added Pd/C (92 mg, 0.086 mmol) and hydrogenated using a balloon overnight at room temperature. The reaction mixture was filtered through Celite and filtrate was concentrated in vacuum. The residue obtained was purified by flash column chromatography (silica gel (12 g), eluting with 0 to 25% ethyl acetate in hexanes) to give ethyl 2-(2-hydroxy-4-((isopropoxycarbonyl)amino)phenyl)acetate (20d) (245 mg, 81% yield) as a white solid; ¹H NMR (300 MHz, DMSO-d₆) δ 9.46 (s, 1H), 9.40 (s, 1H), 7.09 (d, J=2.0 Hz, 1H), 6.94 (d, J=8.2 Hz, 1H), 6.77 (dd, J=8.2, 2.1 Hz, 1H), 4.96-4.74 (m, 1H), 4.03 (q, J=7.1 Hz, 2H), 3.43 (s, 2H), 1.24 (d, J=6.2 Hz, 6H), 1.16 (t, J=7.1 Hz, 3H); MS (ES−): 280.00 (M−1).

### Step-4: Preparation of 7-bromo-5-(chloromethyl)benzofuran (20e)

To a solution of (7-bromobenzofuran-5-yl)methanol (1b) (5.00 g, 22.02 mmol) in DCM (100 mL) was added SOCl₂ (3.21 mL, 44.0 mmol), a drop of DMF (0.3 mL) and stirred reaction at room temperature for 5 h. The reaction mixture was concentrated in vacuum to afford 7-bromo-5-(chloromethyl)benzofuran (20e) (4.88 g, 90% yield) as an off-white solid; ¹H NMR (300 MHz, DMSO-d₆) δ 8.16 (d, J=2.2 Hz, 1H), 7.77 (d, J=1.6 Hz, 1H), 7.66 (d, J=1.6 Hz, 1H), 7.12 (d, J=2.2 Hz, 1H), 4.88 (s, 2H).

### Step-5: Preparation of ethyl 2-(2-((7-bromobenzofuran-5-yl)methoxy)-4-((isopropoxycarbonyl)amino)phenyl)acetate (20f)

Compound 20f was prepared according to the procedure reported in step-4 of scheme 14 from 7-bromo-5-(chloromethyl)benzofuran (20e) (201 mg, 0.818 mmol) using ethyl 2-(2-hydroxy-4-((isopropoxycarbonyl)amino)phenyl)acetate (20d) (230 mg, 0.818 mmol), K₂CO₃ (339 mg, 2.453 mmol) in DMF (10 mL) and stirring at room temperature for 16h. This gave after workup and purification by flash column chromatography (SiO₂ (24 g), eluting with 0 to 20% EtOAc in hexane) ethyl 2-(2-((7-bromobenzofuran-5-yl)methoxy)-4-((isopropoxycarbonyl)amino)phenyl)acetate (20f) (311 mg, 78%)¹H NMR (300 MHz, DMSO-d₆) δ 9.55 (s, 1H), 8.15 (d, J=2.2 Hz, 1H), 7.71 (d, J=1.5 Hz, 1H), 7.60 (d, J=1.4 Hz, 1H), 7.32-7.29 (m, 1H), 7.13-7.05 (m, 2H), 6.99-6.94 (m, 1H), 5.10 (s, 2H), 4.96-4.77 (m, 1H), 4.00 (q, J=7.1 Hz, 2H), 3.53 (s, 2H), 1.24 (d, J=6.2 Hz, 6H), 1.07 (t, J=7.1 Hz, 3H); MS (ES+): 489.90 (M+1).

### Step-6: Preparation of ethyl 2-(2-((7-(3-(aminomethyl)phenyl)benzofuran-5-yl)methoxy)-4-((isopropoxycarbonyl)amino)phenyl)acetate (20g)

Compound 20g was prepared according to the procedure reported in step-4 of scheme 1 from ethyl 2-(2-((7-bromobenzofuran-5-yl)methoxy)-4-((isopropoxycarbonyl)amino)phenyl)acetate (20f) (300 mg, 0.612 mmol) in dioxane (10 mL) using 3-(aminomethyl)phenylboronic acid hydrochloride (9e) (139 mg, 0.918 mmol), a solution of potassium bicarbonate (254 mg, 1.835 mmol) in water (1 mL) and bis(triphenylphosphine)palladium(II) chloride (64.4 mg, 0.092 mmol) and heating under a nitrogen atmosphere at 100° C. for 4 h on an oil bath. This gave after workup, purification by flash column chromatography [silica gel]ethyl 2-(2-((7-(3-(aminomethyl)phenyl)benzofuran-5-yl)methoxy)-4-((isopropoxycarbonyl)amino)phenyl)acetate (20g) (181 mg, 57%); MS (ES+): 517.10 (M+1).

### Step-7: Preparation of 2-(2-((7-(3-(aminomethyl)phenyl)benzofuran-5-yl)methoxy)-4-((isopropoxycarbonyl)amino)phenyl)acetic acid (20h)

Compound 20h was prepared according to procedure reported in Step-8 of scheme 1 from ethyl 2-(2-((7-(3-(aminomethyl)phenyl)benzofuran-5-yl)methoxy)-4-((isopropoxycarbonyl)amino)phenyl)acetate (20g) (160 mg, 0.310 mmol) in THE (5 mL), methanol (5 mL) using a solution of lithium hydroxide monohydrate (80 mg, 1.858 mmol) in water (5 mL) and stirring for 16 h at room temperature. This gave after workup and purification by reverse-phase column chromatography [C-18 column, eluting with 0.1% aq HCl in water and acetonitrile from 0-100%]2-(2-((7-(3-(aminomethyl)phenyl)benzofuran-5-yl)methoxy)-4-((isopropoxycarbonyl)amino)phenyl)acetic acid (20h) (84 mg, 55.5% yield) HCl salt as a white solid; ¹H NMR (300 MHz, DMSO-d₆) δ 12.10 (s, 1H, D₂O exchangeable), 9.55 (s, 1H), 8.36 (s, 3H, D₂O exchangeable), 8.11 (d, J=2.2 Hz, 1H), 8.02-7.98 (m, 1H), 7.95 (dt, J=7.4, 1.6 Hz, 1H), 7.77 (d, J=1.6 Hz, 1H), 7.64 (d, J=1.6 Hz, 1H), 7.62-7.52 (m, 2H), 7.39 (s, 1H), 7.11-7.04 (m, 2H), 6.94 (dd, J=8.1, 1.9 Hz, 1H), 5.19 (s, 2H), 4.96-4.79 (m, 1H), 4.14 (s, 2H), 3.51 (s, 2H), 1.24 (d, J=6.2 Hz, 6H); MS (ES+): 489.2 (M+1); (ES−): 487.2 (M−1); Analysis calculated for C₂₈H₂₈N₂O₆·HCl·1.5H₂O: C, 60.92; H, 5.84; Cl, 6.42; N, 5.07; Found: C, 61.09; H, 5.69; Cl, 6.55; N, 5.16.

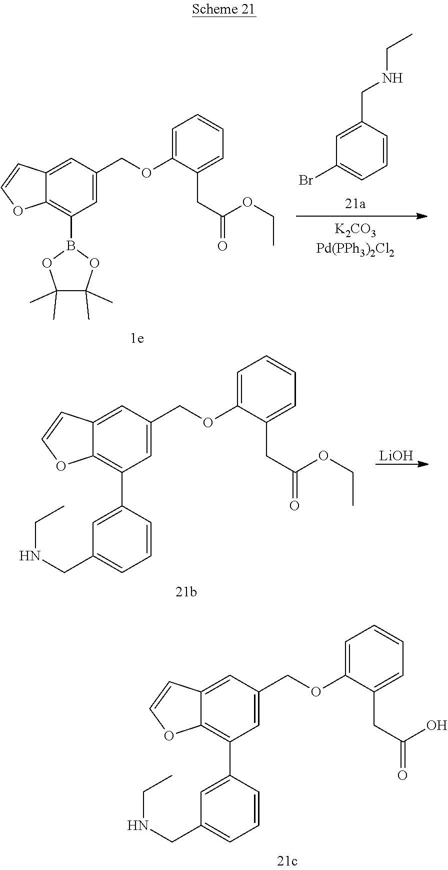

### Preparation of 2-(2-((7-(3-((ethylamino)methyl)phenyl)benzofuran-5-yl)methoxy)phenyl)acetic acid (21c)

### Step-1: Preparation of ethyl 2-(2-((7-(3-((ethylamino)methyl)phenyl)benzofuran-5-yl)methoxy)phenyl)acetate (21b)

Compound 21b was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (300 mg, 0.688 mmol) in dioxane (5 mL) using N-(3-bromobenzyl)ethanamine (21a) (221 mg, 1.031 mmol; CAS #90389-91˜6), bis(triphenylphosphine)Palladium(II)chloride [Pd(PPh₃)₂Cl₂](72.4 mg, 0.103 mmol), a solution of K₂CO₃ (285 mg, 2.063 mmol) in water (3 mL) and heating at 135° C. for 30 min on microwave. This gave after workup and purification by flash column chromatography [silica gel (12 g), eluting with DMA-80 in DCM from 0-50%]ethyl 2-(2-((7-(3-((ethylamino)methyl)phenyl)benzofuran-5-yl)methoxy)phenyl)acetate (21b) (122 mg, 40% yield) as a yellow oil. MS (ES+): 444.2 (M+1).

### Step-2: Preparation of 2-(2-((7-(3-((ethylamino)methyl)phenyl)benzofuran-5-yl)methoxy)phenyl)acetic acid (21c)

Compound 21c was prepared according to procedure reported in Step-8 of scheme 1 from ethyl 2-(2-((7-(3-((ethylamino)methyl)phenyl)benzofuran-5-yl)methoxy)phenyl)acetate (21b) (122 mg, 0.275 mmol) in THE (3 mL), using a solution of lithium hydroxide hydrate (87 mg, 2.063 mmol) in water (1 mL) and heating at 50° C. overnight. This gave after workup and purification by reverse-phase column chromatography [C-18 column (40 g), eluting with 0.1% aq HCl in water and acetonitrile from 0-100%]2-(2-((7-(3-((ethylamino)methyl)phenyl)benzofuran-5-yl)methoxy)phenyl)acetic acid (21c) (22 mg, 6.81% yield) as a white solid. ¹H NMR (300 MHz, DMSO-d₆) δ 12.18 (s, 1H, D₂O exchangeable), 9.12 (s, 2H, D₂O exchangeable), 8.05 (d, J=2.2 Hz, 1H), 7.99 (d, J=1.9 Hz, 1H), 7.93-7.83 (m, 1H), 7.70 (d, J=1.6 Hz, 1H), 7.60 (d, J=1.6 Hz, 1H), 7.58-7.52 (m, 2H), 7.21-7.12 (m, 2H), 7.06-6.97 (m, 2H), 6.84 (td, J=7.4, 1.1 Hz, 1H), 5.21 (s, 2H), 4.16 (s, 2H), 3.54 (s, 2H), 2.93 (q, J=7.3 Hz, 2H), 1.18 (t, J=7.2 Hz, 3H); MS (ES+): 416.1 (M+1).

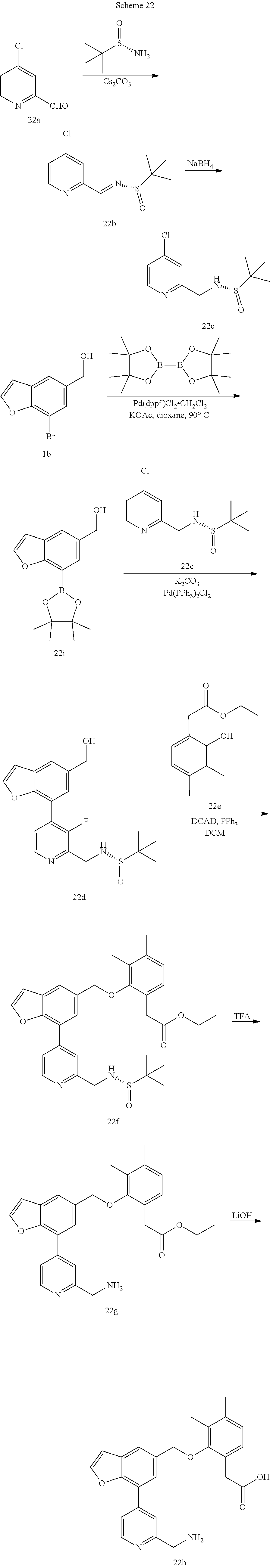

### Preparation of 2-(2-((7-(2-(aminomethyl)pyridin-4-yl)benzofuran-5-yl)methoxy)-3,4-dimethylphenyl)acetic acid (22h)

### Step-1: Preparation of N-((4-chloropyridin-2-yl)methylene)-2-methylpropane-2-sulfinamide (22b)

Compound 22b was prepared according to the procedure reported in step-1 of scheme 11 from 4-chloropicolinaldehyde (22a) (15 g, 106 mmol) in DCM (100 mL) using Cs₂CO₃ (51.8 g, 159 mmol), (S)-2-methylpropane-2-sulfinamide (14.77 g, 122 mmol) and stirring at room temperature for 1 h. This gave after workup N-((4-chloropyridin-2-yl)methylene)-2-methylpropane-2-sulfinamide (22b) (25.9 g, 106 mmol, 100% yield) which was used as such in the next step; ¹H NMR (300 MHz, DMSO-d₆) δ 8.75 (dd, J=5.3, 0.6 Hz, 1H), 8.48 (s, 1H), 8.13 (dd, J=2.1, 0.6 Hz, 1H), 7.76 (dd, J=5.3, 2.1 Hz, 1H), 1.22 (s, 9H).

### Step-2: Preparation of (+)-N-((4-chloropyridin-2-yl)methyl)-2-methylpropane-2-sulfinamide (22c)

Compound 22c was prepared according to the procedure reported in step-2 of scheme 11 from N-((4-chloropyridin-2-yl)methylene)-2-methylpropane-2-sulfinamide (22b) (18.5 g, 76 mmol) in methanol (300 mL) using NaBH₄ (2.86 g, 76 mmol). This gave after workup and purification by flash column chromatography [silica gel (120 g), eluting with a 9:1 mixture of ethyl acetate and methanol in hexanes](+)-N-((4-chloropyridin-2-yl)methyl)-2-methylpropane-2-sulfinamide (22c) (15.7 g, 84%) as a white solid. ¹H NMR (300 MHz, DMSO-d₆) δ 8.48 (dd, J=5.3, 0.6 Hz, 1H), 7.58 (dd, J=2.1, 0.7 Hz, 1H), 7.43 (dd, J=5.4, 2.1 Hz, 1H), 5.97 (t, J=6.3 Hz, 1H), 4.29 (dd, J=6.3, 3.3 Hz, 2H), 1.16 (s, 9H); Optical rotation [α]_{D}=+45.4 (0.81, MeOH).

### Step-3: Preparation of (7-(4,4,5,5-tetramethyl-1,3,2-dioxaborolan-2-yl)benzofuran-5-yl)methanol (22i)

Compound 22i was prepared according to procedure reported in Step-3 of scheme 1 from (7-bromobenzofuran-5-yl)methanol (1b) (6.5 g, 28.6 mmol), using bis(pinacolato)diboron (10.9 g, 42.9 mmol), potassium acetate (8.43 g, 86 mmol) and PdCl₂(dppf)-CH₂Cl₂ (2.34 g, 2.86 mmol) in anhydrous dioxane (200 mL) under an Argon atmosphere and heating at 90° C. for 18h. This gave after workup and purification by flash column chromatography [silica (120 g), eluting with EtOAc in hexane from 0-60%](7-(4,4,5,5-tetramethyl-1,3,2-dioxaborolan-2-yl)benzofuran-5-yl)methanol (22i) (6.81 g, 87% yield) as an off-white solid; ¹H NMR (300 MHz, DMSO-d₆) δ 8.01 (d, J=2.2 Hz, 1H), 7.71 (dd, J=1.8, 0.9 Hz, 1H), 7.59 (d, J=2.2 Hz, 1H), 6.93 (d, J=2.2 Hz, 1H), 5.22 (t, J=5.8 Hz, 1H), 4.58 (dt, J=5.8, 0.7 Hz, 2H), 1.34 (s, 12H).

### Step-4: Preparation of (+)-N-((4-(5-(hydroxymethyl)benzofuran-7-yl)pyridin-2-yl)methyl)-2-methylpropane-2-sulfinamide (22d)

Compound 22d was prepared according to the procedure reported in step-4 of scheme 1 from (7-(4,4,5,5-tetramethyl-1,3,2-dioxaborolan-2-yl)benzofuran-5-yl)methanol (22i) (1.5 g, 5.47 mmol) in dioxane (60 mL) using (+)-N-((4-chloropyridin-2-yl)methyl)-2-methylpropane-2-sulfinamide (22c) (1.620 g, 6.57 mmol), PdCl₂(PPh₃)₂ (0.576 g, 0.821 mmol) and a solution of potassium carbonate (2.269 g, 16.42 mmol) in water (7 mL) under a nitrogen atmosphere heating at 100° C. for 16 h on oil bath. This gave after workup and purification by flash column chromatography [silica gel (80 g), eluting with methanol in DCM from 0-7%](+)-N-((4-(5-(hydroxymethyl)benzofuran-7-yl)pyridin-2-yl)methyl)-2-methylpropane-2-sulfinamide (22d) (875 mg, 45% yield) as an off-white gummy solid; ¹H NMR (300 MHz, DMSO-d₆) δ 8.63 (dd, J=5.2, 0.8 Hz, 1H), 8.07 (d, J=2.2 Hz, 1H), 8.04-8.03 (m, 1H), 7.78 (dd, J=5.2, 1.8 Hz, 1H), 7.72-7.69 (m, 1H), 7.62 (d, J=1.6 Hz, 1H), 7.07 (d, J=2.2 Hz, 1H), 5.98 (t, J=6.1 Hz, 1H), 5.31 (t, J=5.7 Hz, 1H), 4.65 (dt, J=5.7, 0.7 Hz, 2H), 4.45-4.28 (m, 2H), 1.19 (s, 9H); MS (ES+): 359.10 (M+1); Optical rotation [α]_{D}=+32.77 (c=0.47, MeOH).

### Step-5: Preparation of (+)-ethyl 2-(2-((7-(2-((1,1-dimethylethylsulfinamido)methyl)pyridin-4-yl)benzofuran-5-yl)methoxy)-3,4-dimethylphenyl)acetate (22f)

Compound 22f was prepared according to procedure reported in Step-2 of scheme 1 from (+)-N-((4-(5-(hydroxymethyl)benzofuran-7-yl)pyridin-2-yl)methyl)-2-methylpropane-2-sulfinamide (22d) (0.861 g, 2.401 mmol) in DCM (40 mL) using triphenylphosphine (0.945 g, 3.60 mmol), ethyl 2-(2-hydroxy-3,4-dimethylphenyl)acetate (22e) (CAS #: 1806292-81-8, 0.5 g, 2.401 mmol) and a solution of (E)-bis(4-chlorobenzyl) diazene-1,2-dicarboxylate (DCAD) (1.322 g, 3.60 mmol) in DCM (40 mL) and stirring at room temperature for 22 h. This gave after workup and purification by flash column chromatography (silica gel (40 g), eluting with 0 to 100% ethyl acetate in hexanes) (+)-ethyl 2-(2-((7-(2-((1,1-dimethylethylsulfinamido)methyl)pyridin-4-yl)benzofuran-5-yl)methoxy)-3,4-dimethylphenyl)acetate (22f) (145 mg, 11.01% yield) as a clear oil. MS (ES+): 549.2 (M+1); Optical rotation [α]_{D}=+15.238 (c=0.105, MeOH).

### Step-6: Preparation of ethyl 2-(2-((7-(2-(aminomethyl)pyridin-4-yl)benzofuran-5-yl)methoxy)-3,4-dimethylphenyl)acetate (22g)

To a solution of (+)-ethyl 2-(2-((7-(2-((1,1-dimethylethylsulfinamido)methyl)pyridin-4-yl)benzofuran-5-yl)methoxy)-3,4-dimethylphenyl)acetate (22f) (140 mg, 0.255 mmol) in THE (150 mL) was treated with HCl (3 N) (0.255 mL, 0.765 mmol) at room temperature and stirred for 6 h. The reaction mixture was concentrated to produce ethyl 2-(2-((7-(2-(aminomethyl)pyridin-4-yl)benzofuran-5-yl)methoxy)-3,4-dimethylphenyl)acetate (22g) (140 mg, 0.315 mmol, 123% yield) as a clear oil, which was used in the next step without purification, MS (ES+) 445.2 (M+1).

### Step-7: Preparation of 2-(2-((7-(2-(aminomethyl)pyridin-4-yl)benzofuran-5-yl)methoxy)-3,4-dimethylphenyl)acetic acid (22h)

Compound 22h was prepared according to procedure reported in Step-8 of scheme 1 from ethyl 2-(2-((7-(2-(aminomethyl)pyridin-4-yl)benzofuran-5-yl)methoxy)-3,4-dimethylphenyl)acetate (22g) (140 mg, 0.315 mmol) in THE (6 mL), methanol (1 mL) using a solution of lithium hydroxide monohydrate (108 mg, 2.52 mmol) in water (1 mL) and stirring for 19 h at room temperature. This gave after workup and purification by reverse-phase column chromatography [C-18 column, eluting with 0.1% aq HCl in water and acetonitrile from 0-100%]2-(2-((7-(2-(aminomethyl)pyridin-4-yl)benzofuran-5-yl)methoxy)-3,4-dimethylphenyl)acetic acid (22h) (50 mg, 38% yield) HCl salt as a white solid. ¹H NMR (300 MHz, DMSO-d₆) δ 8.79 (d, J=5.2 Hz, 1H), 8.38 (s, 3H, D₂O exchangeable), 8.19 (d, J=2.3 Hz, 1H), 8.05 (s, 1H), 7.98 (dd, J=5.3, 1.7 Hz, 1H), 7.93 (d, J=1.6 Hz, 1H), 7.82 (d, J=1.6 Hz, 1H), 7.16 (d, J=2.2 Hz, 1H), 7.03 (d, J=7.6 Hz, 1H), 6.94 (d, J=7.8 Hz, 1H), 4.91 (s, 2H), 4.37-4.27 (m, 2H), 3.60 (s, 2H), 2.24 (s, 3H), 2.22 (s, 3H); MS (ES+): 417.2 (M+1); (ES−): 415.2 (M−1); Analysis calculated for C₂₅H₂₄N₂O₄·1.5HCl·2H₂O: C, 59.20; H, 5.86; Cl, 10.49; N, 5.52; Found: C, 59.05; H, 5.59; Cl, 10.21; N, 5.44.

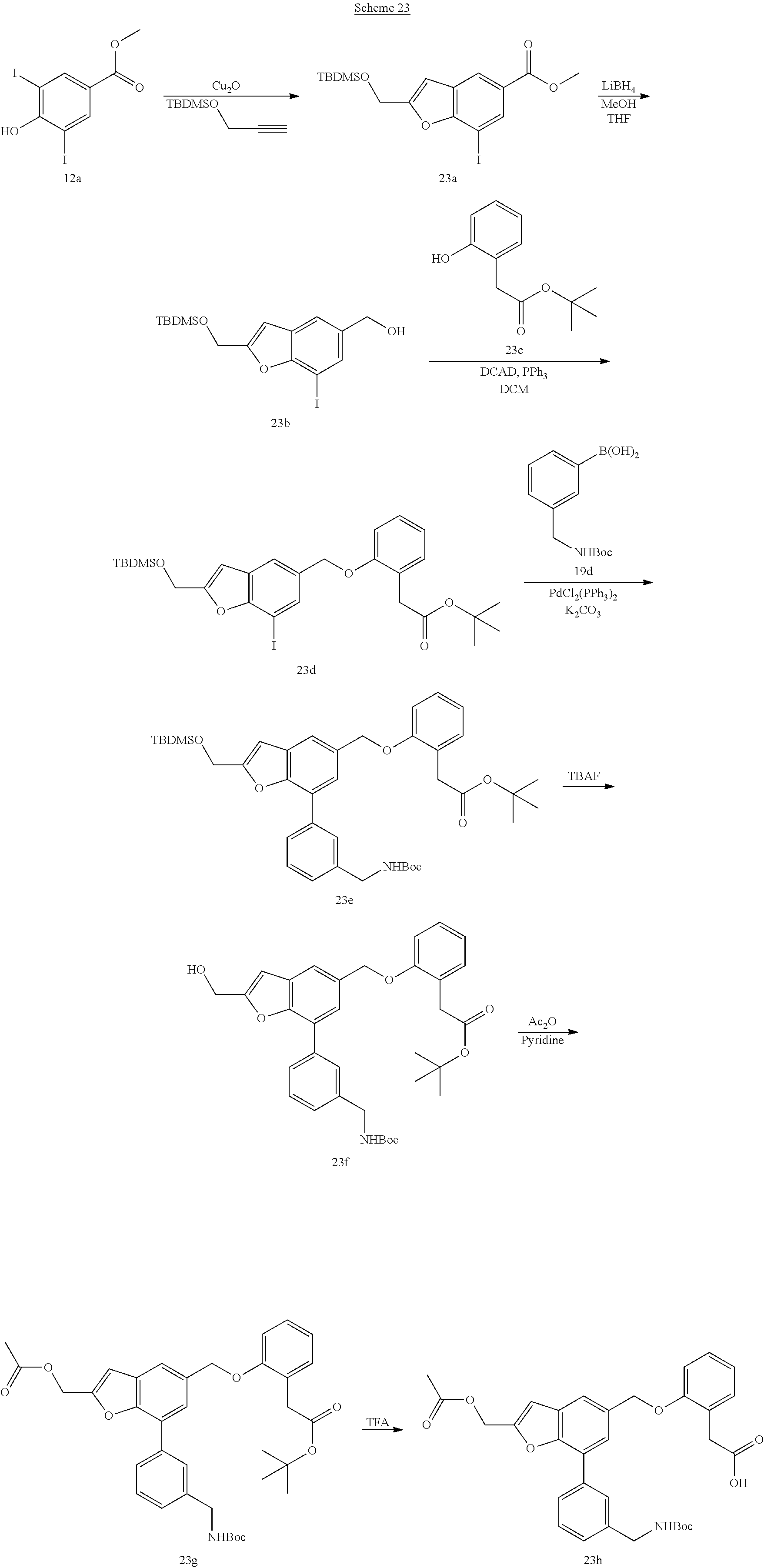

### Preparation of 2-(2-((2-(acetoxymethyl)-7-(3-(aminomethyl)phenyl)benzofuran-5-yl)methoxy)phenyl)acetic acid (23h)

### Step-1: Preparation of methyl 2-(((tert-butyldimethylsilyl)oxy)methyl)-7-iodobenzofuran-5-carboxylate (23a)

Compound 23a was prepared according to the procedure reported in step-1 of scheme 12, from methyl 4-hydroxy-3,5-diiodobenzoate (12a) (5 g, 12.38 mmol) in pyridine (10 mL) using tert-butyldimethyl(prop-2-ynyloxy)silane (2.11 g, 12.38 mmol; CAS #76782-82-6) and copper(I) oxide (0.89 g, 6.19 mmol). This gave after workup and purification by flash column chromatography [silica (80 g), eluting with EtOAc in hexane from 0-70%]methyl 2-(((tert-butyldimethylsilyl)oxy)methyl)-7-iodobenzofuran-5-carboxylate (23a) (3.2 g, 58% yield) as a clear oil; ¹H NMR (300 MHz, DMSO-d₆) δ 8.13 (d, J=1.6 Hz, 1H), 8.07 (d, J=1.6 Hz, 1H), 6.95 (s, 1H), 4.72 (s, 2H), 3.74 (s, 3H), 0.76 (s, 9H), −0.00 (s, 6H); MS (ES+): 469.1 (M+Na).

### Step-2: Preparation of (2-(((tert-butyldimethylsilyl)oxy)methyl)-7-iodobenzofuran-5-yl)methanol (23b)

Compound 23b was prepared according to the procedure reported in step-5 of scheme 11, from methyl 2-(((tert-butyldimethylsilyl)oxy)methyl)-7-iodobenzofuran-5-carboxylate (23a) (12 g, 26.9 mmol) in THE (150 mL) using LiBH₄ (26.9 mL, 53.8 mmol, 2 M solution in THF) and MeOH (2.2 mL, 53.8 mmol). This gave after workup and purification by flash column chromatography [silica (80 g), eluting with EtOAc in hexane from 0-60%] to afford (2-(((tert-butyldimethylsilyl)oxy)methyl)-7-iodobenzofuran-5-yl)methanol (23b) (10.4 g, 92% yield) as a clear oil; ¹H NMR (300 MHz, DMSO-d₆) δ 7.62 (d, J=1.5 Hz, 1H), 7.52 (d, J=1.4 Hz, 1H), 6.92 (d, J=1.2 Hz, 1H), 5.25 (t, J=5.8, 1.2 Hz, 1H, D₂O exchangeable), 4.81 (s, 2H), 4.53 (d, J=5.8 Hz, 2H), 0.89 (s, 9H), 0.12 (s, 6H); MS (ES+): 441.2 (M+Na); (ES−): 417.2 (M−1).

### Step-3: Preparation of tert-butyl 2-(2-((2-(((tert-butyldimethylsilyl)oxy)methyl)-7-iodobenzofuran-5-yl)methoxy)phenyl)acetate (23d)

Compound 23d was prepared according to procedure reported in Step-2 of scheme 1 from (2-(((tert-butyldimethylsilyl)oxy)methyl)-7-iodobenzofuran-5-yl)methanol (23b) (5 g, 11.95 mmol) in DCM (20 mL) using triphenylphosphine (3.45 g, 13.15 mmol), tert-butyl 2-(2-hydroxyphenyl)acetate (23c) (2.74 g, 13.15 mmol) a solution of (E)-bis(4-chlorobenzyl) diazene-1,2-dicarboxylate (DCAD) (4.83 g, 13.15 mmol) in DCM (20 mL) and stirring at room temperature for 1 h. This gave after workup and purification by flash column chromatography (silica gel (40 g), eluting with 0 to 50% ethyl acetate in hexanes) tert-butyl 2-(2-((2-(((tert-butyldimethylsilyl)oxy)methyl)-7-iodobenzofuran-5-yl)methoxy)phenyl)acetate (23d) (3.58 g, 49% yield) as a clear oil; ¹H NMR (300 MHz, DMSO-d₆) δ 7.75 (d, J=1.5 Hz, 1H), 7.67 (s, 1H), 7.26-7.15 (m, 2H), 7.05 (d, J=8.2 Hz, 1H), 6.96-6.86 (m, 2H), 5.14 (s, 2H), 4.83 (s, 2H), 3.53 (s, 2H), 1.29 (s, 9H), 0.89 (s, 9H), 0.12 (s, 6H).

### Step-4: Preparation of tert-butyl 2-(2-((7-(3-(((tert-butoxycarbonyl)amino)methyl)phenyl)-2-(((tert-butyldimethylsilyl)oxy)methyl)benzofuran-5-yl)methoxy)phenyl)acetate (23e)

Compound 23e was prepared according to procedure reported in Step-4 of scheme 1 from tert-butyl 2-(2-((2-(((tert-butyldimethylsilyl)oxy)methyl)-7-iodobenzofuran-5-yl)methoxy)phenyl)acetate (23d) (3.25 g, 5.34 mmol) in dioxane (35 mL) using 3-((tert-butoxycarbonylamino)methyl)phenylboronic acid (19d) (2.011 g, 8.01 mmol), bis(triphenylphosphine)Palladium(II)chloride [Pd(PPh₃)₂Cl₂](0.562 g, 0.801 mmol), a solution of K₂CO₃ (1.476 g, 10.68 mmol) in water (3 mL) and heating at 100° C. for 3 h on oil bath. This gave after workup and purification by flash column chromatography [silica gel (12 g), eluting with DMA-80 in DCM from 0-50%] tert-butyl 2-(2-((7-(3-(((tert-butoxycarbonyl)amino)methyl)phenyl)-2-(((tert-butyldimethylsilyl)oxy)methyl)benzofuran-5-yl)methoxy)phenyl)acetate (23e) (3.4 g, 93% yield) as a yellow oil; ¹H NMR (300 MHz, DMSO-d₆) δ 7.80-7.70 (m, 2H), 7.68 (d, J=1.6 Hz, 1H), 7.55 (d, J=1.6 Hz, 1H), 7.49-7.43 (m, 2H), 7.29 (d, J=8.2 Hz, 1H), 7.26-7.17 (m, 2H), 7.09 (dd, J=8.3, 1.2 Hz, 1H), 6.91 (dd, J=7.4, 1.1 Hz, 1H), 6.89-6.85 (m, 1H), 5.22 (s, 2H), 4.83 (s, 2H), 4.22 (d, J=6.2 Hz, 2H), 3.54 (s, 2H), 1.39 (s, 9H), 1.23 (s, 9H), 0.89 (s, 9H), 0.10 (s, 6H).

### Step-5: Preparation of tert-butyl 2-(2-((7-(3-(((tert-butoxycarbonyl)amino)methyl)phenyl)-2-(hydroxymethyl)benzofuran-5-yl)methoxy)phenyl)acetate (23f)

To a solution of tert-butyl 2-(2-((7-(3-(((tert-butoxycarbonyl)amino)methyl)phenyl)-2-(((tert-butyldimethylsilyl)oxy)methyl)benzofuran-5-yl)methoxy)phenyl)acetate (23e) (3.4 g, 4.94 mmol) in THE (35 mL) was added TBAF (1M in THF) (4.94 mL, 4.94 mmol). The mixture was stirred for 3 h at RT. This gave after workup and purification by flash column chromatography (Silica gel, eluting with EtOAc in hexane from 0-100%) tert-butyl 2-(2-((7-(3-(((tert-butoxycarbonyl)amino)methyl)phenyl)-2-(hydroxymethyl)benzofuran-5-yl)methoxy)phenyl)acetate (23f) (2.6 g, 92% yield) as a clear oil. MS (ES+): 596.3 (M+Na).

### Step-6: Preparation of tert-butyl 2-(2-((2-(acetoxymethyl)-7-(3-(((tert-butoxycarbonyl)amino)methyl)phenyl)benzofuran-5-yl)methoxy)phenyl)acetate (23g)

To a solution of tert-butyl 2-(2-((7-(3-(((tert-butoxycarbonyl)amino)methyl)phenyl)-2-(hydroxymethyl)benzofuran-5-yl)methoxy)phenyl)acetate (23f) (650 mg, 1.133 mmol) in DCM (5 mL) was added pyridine (0.137 mL, 1.700 mmol) acetic anhydride (0.107 mL, 1.133 mmol) and stirred at room temperature overnight. The reaction was quenched with KHSO₄ (1N) solution, extracted with EtOAc, dried and concentrated in vacuum. The residue obtained was purified by flash column chromatography [silica (40 g), eluting with EtOAc/MeOH (9:1) in hexane from 0-80%] followed by reverse phase column chromatography [C18 column, eluting with ACN in water from 0-100%] to give tert-butyl 2-(2-((2-(acetoxymethyl)-7-(3-(((tert-butoxycarbonyl)amino)methyl)phenyl)benzofuran-5-yl)methoxy)phenyl)acetate (23g) (352 mg, 0.572 mmol, 50.5% yield) as a clear oil, MS (ES+): 516.1 (M-Boc+1)

### Step-7: Preparation of 2-(2-((2-(acetoxymethyl)-7-(3-(aminomethyl)phenyl)benzofuran-5-yl)methoxy)phenyl)acetic acid (23h)

To a solution of tert-butyl 2-(2-((2-(acetoxymethyl)-7-(3-(((tert-butoxycarbonyl)amino)methyl)phenyl)benzofuran-5-yl)methoxy)phenyl)acetate (23g) (352 mg, 0.572 mmol) in DCM (10 mL) was added TFA (0.087 mL, 1.133 mmol). The resulting mixture was stirred for 3 h at RT and concentrated in vacuum to dryness. The residue was purified by reverse phase column chromatography [C18 (50 g), eluting with ACN in water (containing 0.1% HCl) from 0-100%] to give 2-(2-((2-(acetoxymethyl)-7-(3-(aminomethyl)phenyl)benzofuran-5-yl)methoxy)phenyl)acetic acid (23h) (157 mg, 30.2% yield) HCl salt as a white solid; ¹H NMR (300 MHz, DMSO-d₆) δ 8.64 (s, 2H, D₂O exchangeable), 8.05-7.99 (m, 1H), 7.97-7.89 (m, 1H), 7.74 (d, J=1.6 Hz, 1H), 7.69 (d, J=1.7 Hz, 1H), 7.61-7.55 (m, 2H), 7.23 (dd, J=8.1, 6.5 Hz, 2H), 7.09 (d, J=8.4 Hz, 2H), 6.90 (td, J=7.4, 1.1 Hz, 1H), 5.27 (d, J=2.7 Hz, 4H), 4.13 (s, 2H), 3.60 (s, 2H), 2.08 (s, 3H); MS (ES+): 460.1 (M+1); (ES−): 458.1 (M−1); Analysis calculated for C₂₇H₂₅NO₆·HCl·1.25H₂O: C, 62.55; H, 5.54; Cl, 6.84; N, 2.70; Found: C, 62.43; H, 5.56; Cl, 7.00; N, 2.80.

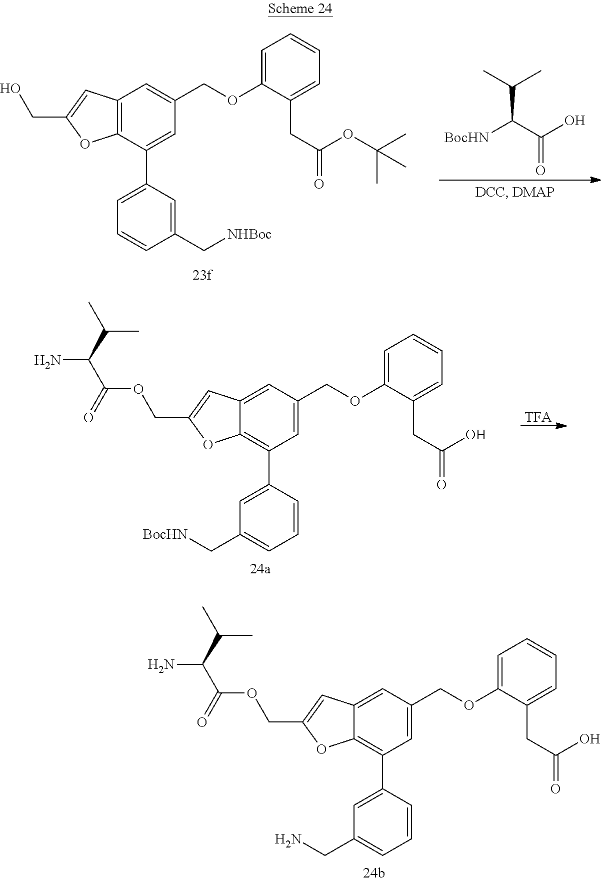 [SMILES: "TFA" ; CC(C)(C)OC(=O)Cc1ccccc1OCc1cc(-c2cccc(CNC(=O)OC(C)(C)C)c2)c2oc(CO)cc2c1 ; CC(C)[C@H](NC(=O)OC(C)(C)C)C(=O)O ; CC(C)[C@H](N)C(=O)OCc1cc2cc(COc3ccccc3CC(=O)O)cc(-c3cccc(CNC(=O)OC(C)(C)C)c3)c2o1 ; CC(C)[C@H](N)C(=O)OCc1cc2cc(COc3ccccc3CC(=O)O)cc(-c3cccc(CN)c3)c2o1]

### Preparation of 2-(2-((2-(((2-amino-3-methylbutanoyl)oxy)methyl)-7-(3-(aminomethyl)phenyl)benzofuran-5-yl)methoxy)phenyl)acetic acid (24b)

### Step-1, Preparation of 2-(2-((2-(((2-amino-3-methylbutanoyl)oxy)methyl)-7-(3-(((tert-butoxycarbonyl)amino)methyl)phenyl)benzofuran-5-yl)methoxy)phenyl)acetic acid (24a)

To a solution of tert-butyl 2-(2-((7-(3-(((tert-butoxycarbonyl)amino)methyl)phenyl)-2-(hydroxymethyl)benzofuran-5-yl)methoxy)phenyl)acetate (23f) (700 mg, 1.220 mmol) and Boc-L-Val-OH (318 mg, 1.464 mmol) in DCM (10 mL) was added DCC (302 mg, 1.464 mmol), DMAP (22.36 mg, 0.183 mmol). The reaction mixture is stirred at room temperature overnight filtered through Celite and concentrated in vacuum. The residue obtained was purified by flash column chromatography [silica (40 g), eluting with EtOAc/MeOH (9:1) in hexane from 0-80%] to give 2-(2-((2-(((2-amino-3-methylbutanoyl)oxy)methyl)-7-(3-(((tert-butoxycarbonyl)amino)methyl)phenyl)benzofuran-5-yl)methoxy)phenyl)acetic acid (24a) (544 mg, 0.704 mmol, 57.7% yield) as a clear oil; ¹H NMR (300 MHz, DMSO-d₆) δ 7.77-7.67 (m, 3H), 7.59 (d, J=1.7 Hz, 1H), 7.51-7.39 (m, 2H), 7.34-7.26 (m, 1H), 7.26-7.15 (m, 3H), 7.12-7.02 (m, 2H), 6.90 (td, J=7.4, 1.1 Hz, 1H), 5.41-5.26 (m, 2H), 5.23 (s, 2H), 4.23 (d, J=6.1 Hz, 2H), 3.88 (t, J=7.2 Hz, 1H), 3.54 (s, 2H), 2.04-1.98 (m, 1H), 1.39 (s, 9H), 1.33 (s, 9H), 1.23 (s, 9H), 0.87-0.80 (m, 6H).

### Step-2, Preparation of 2-(2-((2-(((2-amino-3-methylbutanoyl)oxy)methyl)-7-(3-(aminomethyl)phenyl)benzofuran-5-yl)methoxy)phenyl)acetic acid (24b)

To a solution of 2-(2-((2-(((2-amino-3-methylbutanoyl)oxy)methyl)-7-(3-(((tert-butoxycarbonyl)amino)methyl)phenyl)benzofuran-5-yl)methoxy)phenyl)acetic acid (24a) (544 mg, 0.704 mmol) in DCM (10 mL) was added TFA (0.940 mL, 12.20 mmol). The resulting mixture was stirred for 3 h at RT concentrated in vacuum and purified by reverse phase column chromatography [C18 column (30 g), eluting with ACN in water (containing 0.1% HCl) from 0-100%] to give 2-(2-((2-(((2-amino-3-methylbutanoyl)oxy)methyl)-7-(3-(aminomethyl)phenyl)benzofuran-5-yl)methoxy)phenyl)acetic acid (24b) (240 mg, 38.1% yield) HCl salt as a white solid; ¹H NMR (300 MHz, DMSO-d₆) δ 8.75 (s, 5H, D₂O exchangeable), 8.02 (d, J=2.0 Hz, 1H), 7.97-7.89 (m, 1H), 7.76 (d, J=1.6 Hz, 1H), 7.71 (d, J=1.7 Hz, 1H), 7.63-7.50 (m, 2H), 7.26-7.14 (m, 3H), 7.09 (d, J=8.2 Hz, 1H), 6.94-6.84 (m, 1H), 5.47 (q, J=13.4 Hz, 2H), 5.27 (s, 2H), 4.12 (s, 2H), 3.95 (d, J=4.6 Hz, 1H), 3.60 (s, 2H), 2.29-2.09 (m, 1H), 0.93 (dd, J=16.9, 6.9 Hz, 6H); MS (ES+): 517.2 (M+1); (ES−) 515.6 (M−1); Analysis calculated for C₃₀H₃₂N₂O₆·2HCl·2.25H₂O: C, 57.19; H, 6.16; Cl, 11.25; N, 4.45; Found: C, 57.48; H, 6.01; Cl, 11.08; N, 4.48.

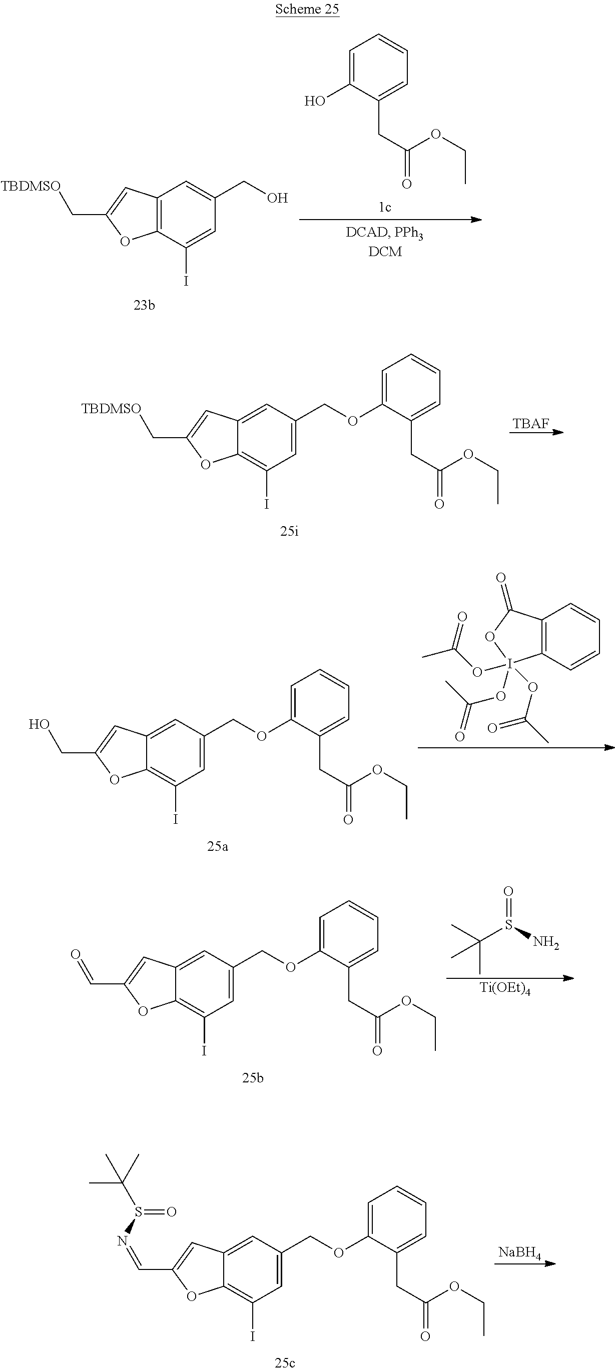

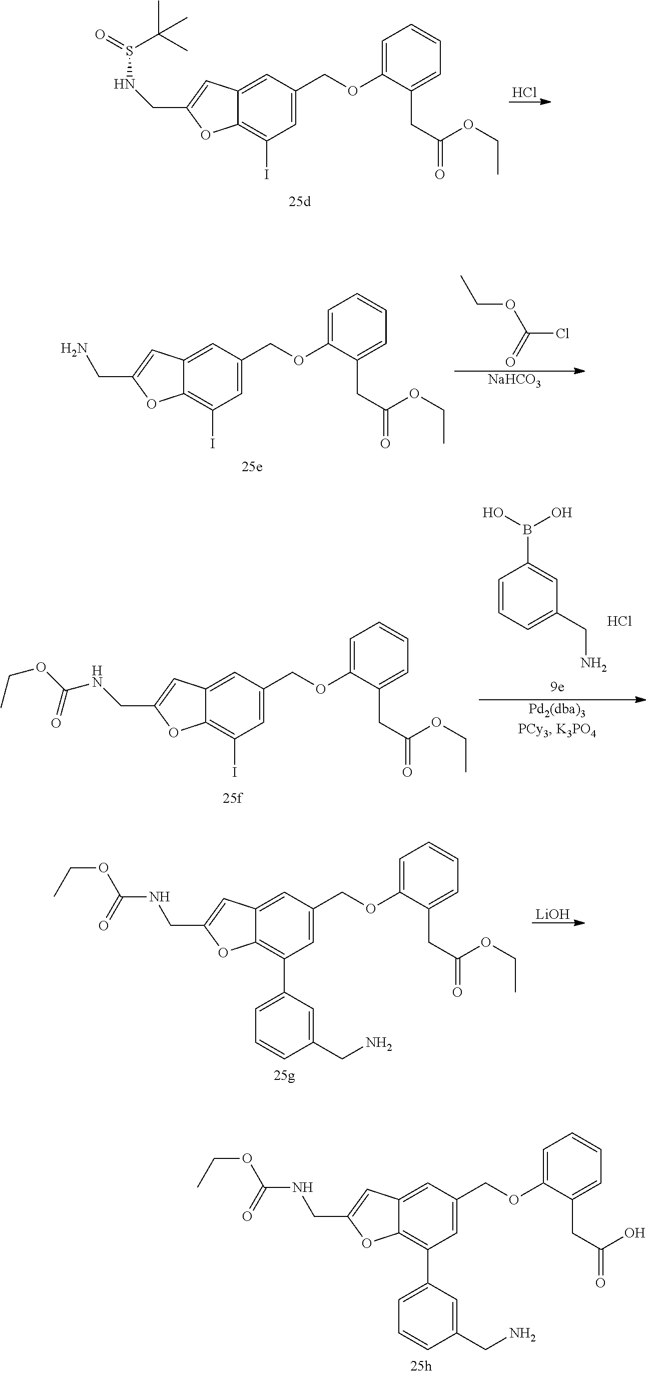

### Preparation of 2-(2-((7-(3-(aminomethyl)phenyl)-2-(((ethoxycarbonyl)amino)methyl)benzofuran-5-yl)methoxy)phenyl)acetic acid (25h)

### Step-1: Preparation of ethyl 2-(2-((2-(((tert-butyldimethylsilyl)oxy)methyl)-7-iodobenzofuran-5-yl)methoxy)phenyl)acetate (25i)

Compound 25i was prepared according to the procedure reported in step-2 of scheme 1 from (2-(((tert-butyldimethylsilyl)oxy)methyl)-7-iodobenzofuran-5-yl)methanol (23b) (2.6 g, 6.22 mmol) in DCM (50 mL) using triphenylphosphine (1.79 g, 6.84 mmol), ethyl 2-(2-hydroxyphenyl) acetate (1c) (1.23 g, 6.84 mmol) and (E)-bis(4-chlorobenzyl) diazene-1,2-dicarboxylate (DCAD, 2.51 g, 6.84 mmol) in DCM (20 mL). This gave after workup and purification by flash column chromatography [silica (40 g), eluting with EtOAc in hexane from 0-50%]ethyl 2-(2-((2-(((tert-butyldimethylsilyl)oxy)methyl)-7-iodobenzofuran-5-yl)methoxy)phenyl)acetate (25i) (2.86 g, 79% yield) as a clear oil; ¹H NMR (300 MHz, DMSO-d₆) δ 7.71 (d, J=1.5 Hz, 1H), 7.64 (d, J=1.5 Hz, 1H), 7.28-7.18 (m, 2H), 7.07 (d, J=8.1 Hz, 1H), 6.96 (s, 1H), 6.95-6.87 (m, 1H), 5.13 (s, 2H), 4.82 (s, 2H), 4.01 (q, J=7.1 Hz, 2H), 3.61 (s, 2H), 1.07 (t, J=7.1 Hz, 3H), 0.89 (s, 9H), 0.12 (s, 6H).

### Step-2: Preparation of ethyl 2-(2-((2-(hydroxymethyl)-7-iodobenzofuran-5-yl)methoxy)phenyl)acetate (25a)

Compound 25a was prepared according to the procedure reported in step-5 of scheme 23 from ethyl 2-(2-((2-(((tert-butyldimethylsilyl)oxy)methyl)-7-iodobenzofuran-5-yl)methoxy)phenyl)acetate (25i) (4 g, 6.89 mmol) in THE (60 mL) using TBAF (2.25 g, 8.61 mmol). This gave after workup, purification by flash column chromatography [silica (24g), eluting with EtOAc in hexane from 0-70%]ethyl 2-(2-((2-(hydroxymethyl)-7-iodobenzofuran-5-yl)methoxy)phenyl)acetate (25a) (2.5 g, 78% yield) as a clear oil; ¹H NMR (300 MHz, DMSO-d₆) δ 7.70 (d, J=1.6 Hz, 1H), 7.63 (d, J=1.5 Hz, 1H), 7.29-7.18 (m, 2H), 7.07 (d, J=8.1 Hz, 1H), 6.95-6.87 (m, 2H), 5.54 (t, J=5.9 Hz, 1H), 5.14 (s, 2H), 4.60 (d, J=5.9 Hz, 2H), 4.04 (q, J=7.1 Hz, 2H), 3.61 (s, 2H), 1.09 (t, J=7.1 Hz, 3H); MS (ES+): 489.1 (M+Na).

### Step-3: Preparation of ethyl 2-(2-((2-formyl-7-iodobenzofuran-5-yl)methoxy)phenyl)acetate (25b)

To a solution of ethyl 2-(2-((2-(hydroxymethyl)-7-iodobenzofuran-5-yl)methoxy)phenyl)acetate (25a) (500 mg, 1.07 mmol) in DCM (20 mL) was added Dess-Martin Periodinane (546 mg, 1.29 mmol). The resulting mixture was stirred at RT for 3h, diluted with dichloromethane, washed with saturated aqueous sodium bicarbonate, dried, filtered, and evaporated in vacuo. The crude product was purified by flash column chromatography [silica gel (24 g)] to afford ethyl 2-(2-((2-formyl-7-iodobenzofuran-5-yl)methoxy)phenyl)acetate (25b) (410 mg, 82% yield) as a white solid; ¹H NMR (300 MHz, DMSO-d₆) δ 9.88 (s, 1H), 8.12 (s, 1H), 8.02 (d, J=1.6 Hz, 1H), 7.93 (d, J=1.6 Hz, 1H), 7.31-7.18 (m, 2H), 7.08 (d, J=8.0 Hz, 1H), 6.93 (t, J=7.4, 1.1 Hz, 1H), 5.20 (s, 2H), 4.03 (q, J=7.1, 1.7 Hz, 2H), 3.63 (s, 2H), 1.09 (t, J=7.1, 1.7 Hz, 3H).

### Step-4: Preparation of (+)-ethyl 2-(2-((2-(((tert-butylsulfinyl)imino)methyl)-7-iodobenzofuran-5-yl)methoxy)phenyl)acetate (25c)

Compound 25c was prepared according to the procedure reported in step-1 of scheme 258 from ethyl 2-(2-((2-formyl-7-iodobenzofuran-5-yl)methoxy)phenyl)acetate (25b) (1.2 g, 2.58 mmol) and (+)-2-methylpropane-2-sulfinamide (0.394 g, 3.23 mmol) in tetrahydrofuran (15 mL) using tetraethoxytitanium (1.084 mL, 5.17 mmol) and heating at reflux for 14 h. This gave after workup and purification by flash column chromatography [silica gel, eluting with ethyl acetate in hexanes (1:0 to 2:1)](+)-ethyl 2-(2-((2-(((tert-butylsulfinyl)imino)methyl)-7-iodobenzofuran-5-yl)methoxy)phenyl)acetate (25c) (965 mg, 66%) as a yellow gum; ¹H NMR (300 MHz, DMSO-d₆) δ 8.55 (s, 1H), 7.96 (s, 1H), 7.93 (d, J=1.5 Hz, 1H), 7.85 (d, J=1.5 Hz, 1H), 7.30-7.18 (m, 2H), 7.07 (dd, J=8.3, 1.1 Hz, 1H), 6.92 (td, J=7.4, 1.1 Hz, 1H), 5.18 (s, 2H), 4.15-3.94 (m, 2H), 3.63 (s, 2H), 1.21 (s, 9H), 1.09 (t, J=7.1 Hz, 3H); Optical rotation [α]_{D}=+61.76 (c=0.34, MeOH)

### Step-5: Preparation of (−)-ethyl 2-(2-((2-((1,1-dimethylethylsulfinamido)methyl)-7-iodobenzofuran-5-yl)methoxy)phenyl)acetate (25d)

Compound 25d was prepared according to the procedure reported in step-3 of scheme 5 from (+)-ethyl 2-(2-((2-(((tert-butylsulfinyl)imino)methyl)-7-iodobenzofuran-5-yl)methoxy)phenyl)acetate (25c) (0.92 g, 1.621 mmol) in tetrahydrofuran (25 mL) using sodium borohydride (0.125 g, 3.24 mmol). This gave after workup and purification by flash column chromatography [silica gel, eluting with hexanes/10% methanol in ethyl acetate (1:0 to 1:1)](−)-ethyl 2-(2-((2-((1,1-dimethylethylsulfinamido)methyl)-7-iodobenzofuran-5-yl)methoxy)phenyl)acetate (25d) (722 mg, 78%) as a colorless gum; ¹H NMR (300 MHz, DMSO-d₆) δ 7.69 (d, J=1.6 Hz, 1H), 7.63 (d, J=1.5 Hz, 1H), 7.30-7.17 (m, 2H), 7.06 (d, J=8.4 Hz, 1H), 6.97-6.86 (m, 2H), 5.99 (t, J=5.7 Hz, 1H), 5.13 (s, 2H), 4.48-4.23 (m, 2H), 4.02 (q, J=7.1 Hz, 2H), 3.61 (s, 2H), 1.15 (s, 9H), 1.08 (t, J=7.1 Hz, 3H); MS (ES+): 570.00 (M+1); Optical rotation [α]_{D}=−10.34 (c=0.29, MeOH)

### Step-6: Preparation of ethyl 2-(2-((2-(aminomethyl)-7-iodobenzofuran-5-yl)methoxy)phenyl)acetate (25e)

To a solution of (−)-ethyl 2-(2-((2-((1,1-dimethylethylsulfinamido)methyl)-7-iodobenzofuran-5-yl)methoxy)phenyl)acetate (25d) (2.68 g, 4.71 mmol, 95% yield) in DCM (30 mL) was added HCl (4N in dioxane) (4 mL). The resulting mixture was stirred for 3 h at RT and concentrated in vacuum. The residue was purified by flash column chromatography (Silica gel (40 g), eluting with EtOAc in hexane from 0-100%) to give ethyl 2-(2-((2-(aminomethyl)-7-iodobenzofuran-5-yl)methoxy)phenyl)acetate (25e) (2.01 g, 88% yield) as a clear oil; MS (ES+): 488.0 (M+Na).

### Step-7: Preparation of ethyl 2-(2-((2-((ethoxycarbonylamino)methyl)-7-iodobenzofuran-5-yl)methoxy)phenyl)acetate (25f)

To a solution of ethyl 2-(2-((2-(aminomethyl)-7-iodobenzofuran-5-yl)methoxy)phenyl)acetate (25e) (1 g, 2.149 mmol) in THF/H₂O (1:1, 8 mL) at 0° C. was added sodium bicarbonate (0.271 g, 3.22 mmol) and ethyl chloroformate (0.308 mL, 3.22 mmol). The reaction mixture was stirred for overnight at RT. The mixture was then diluted with EtOAc, washed with water and brine. The organic layer was dried over MgSO₄, filtered, and concentrated. The residue was purified by flash column chromatography [silica gel (12g), eluting with EtOAc in hexanes from 0-80%) to provide ethyl 2-(2-((2-((ethoxycarbonylamino)methyl)-7-iodobenzofuran-5-yl)methoxy)phenyl)acetate (25f) (521 mg, 45.1% yield) as an pale yellow oil; ¹H NMR (300 MHz, DMSO-d6) δ 7.86-7.78 (m, 1H), 7.70 (q, J=1.9, 1.1 Hz, 1H), 7.62 (t, J=1.8 Hz, 1H), 7.29-7.19 (m, 2H), 7.06 (d, J=8.1 Hz, 1H), 6.91 (td, J=7.4, 1.1 Hz, 1H), 6.84 (d, J=4.3 Hz, 1H), 5.14 (d, J=2.7 Hz, 2H), 4.36 (d, J=5.9 Hz, 2H), 4.07-3.98 (m, 4H), 3.63 (d, J=7.8 Hz, 2H), 1.18 (td, J=7.1, 1.2 Hz, 4H), 1.09 (t, J=7.0 Hz, 2H).

### Step-8: Preparation of ethyl 2-(2-((7-(3-(aminomethyl)phenyl)-2-((ethoxycarbonylamino)methyl)benzofuran-5-yl)methoxy)phenyl)acetate (25g)

To a degassed solution of ethyl 2-(2-((2-((ethoxycarbonylamino)methyl)-7-iodobenzofuran-5-yl)methoxy)phenyl)acetate (25f) (500 mg, 0.93 mmol) in dioxane (10 mL) was added 3-(aminomethyl)phenylboronic acid hydrochloride (9e) (262 mg, 1.40 mmol), K₃PO₄ (2M) (0.79 mL, 1.58 mmol), tricyclohexylphosphine (78 mg, 0.28 mmol) and Pd₂(dba)₃ (128 mg, 0.14 mmol). The mixture was degassed and filled with Ar, then heated 100° C. for 3 h. The mixture was diluted with EtOAc and washed with water and brine. The organic layer was dried, filtered and concentrated in vacuum. The residue was purified by flash column chromatography [silica gel (12g), eluting with DMA-80 in DCM from 0-70%] to give ethyl 2-(2-((7-(3-(aminomethyl)phenyl)-2-((ethoxycarbonylamino)methyl)benzofuran-5-yl)methoxy)phenyl)acetate (25g) (166 mg, 35% yield), as a clear oil; MS (ES+): 517.2 (M+1).

### Step-9: Preparation of 2-(2-((7-(3-(aminomethyl)phenyl)-2-(((ethoxycarbonyl)amino)methyl)benzofuran-5-yl)methoxy)phenyl)acetic acid (25h)

Compound 25h was prepared according to procedure reported in Step-8 of scheme 1 from ethyl 2-(2-((7-(3-(aminomethyl)phenyl)-2-((ethoxycarbonylamino)methyl)benzofuran-5-yl)methoxy)phenyl)acetate (25g) (166 mg, 0.321 mmol) in THE (3 mL) using a solution of lithium hydroxide monohydrate (78 mg, 1.86 mmol) in water (1 mL) and stirring overnight at room temperature. This gave after workup and purification by reverse-phase column chromatography [C-18 column, eluting with 0.1% aq HCl in water and acetonitrile from 0-100%]2-(2-((7-(3-(aminomethyl)phenyl)-2-(((ethoxycarbonyl)amino)methyl)benzofuran-5-yl)methoxy)phenyl)acetic acid (25h) (107 mg, 24% yield) HCl salt as a white solid; ¹H NMR (300 MHz, DMSO-d₆) δ 12.24 (s, 1H, D₂O exchangeable), 8.52 (s, 2H, D₂O exchangeable), 8.03 (s, 1H), 7.98-7.91 (m, 1H), 7.85 (t, J=5.9 Hz, 1H), 7.69 (d, J=1.6 Hz, 1H), 7.63 (d, J=1.7 Hz, 1H), 7.60-7.53 (m, 2H), 7.27-7.18 (m, 2H), 7.09 (d, J=8.1 Hz, 1H), 6.95-6.86 (m, 1H), 6.77 (s, 1H), 5.25 (s, 2H), 4.39 (d, J=5.9 Hz, 2H), 4.14 (s, 2H), 4.04 (q, J=7.1 Hz, 2H), 3.60 (s, 2H), 1.18 (t, J=7.1 Hz, 3H); MS (ES*) 489.2 (M+1); Analysis calculated for C₂₈H₂₈N₂O₆·HCl·1.25H₂O: C, 61.42; H, 5.80; Cl, 6.48; N, 5.12; Found: C, 61.58; H, 5.57; Cl, 6.40; N, 5.20.

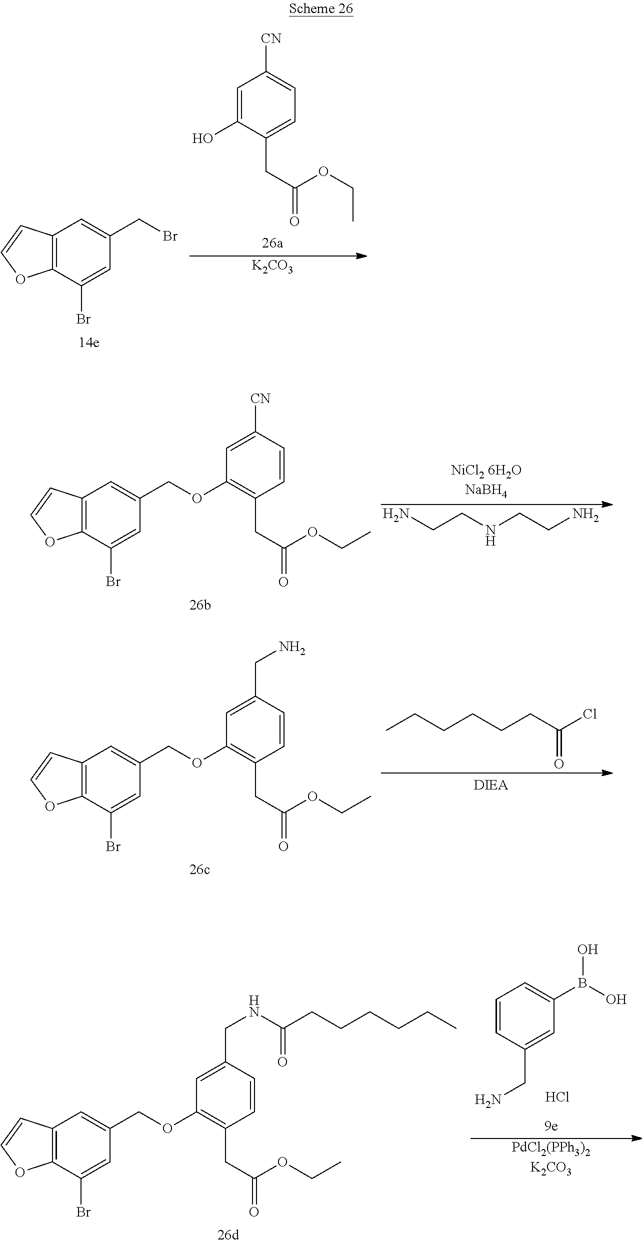

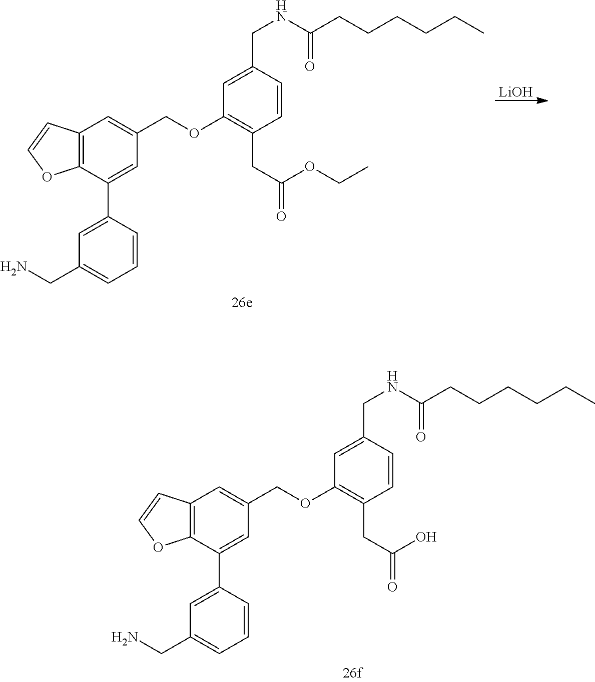 [SMILES: CCCCCCC(=O)NCc1ccc(CC(=O)OCC)c(OCc2cc(-c3cccc(CN)c3)c3occc3c2)c1 ; "LiOH" ; CCCCCCC(=O)NCc1ccc(CC(=O)O)c(OCc2cc(-c3cccc(CN)c3)c3occc3c2)c1]

### Preparation of 2-(2-((7-(3-(aminomethyl)phenyl)benzofuran-5-yl)methoxy)-4-(heptanamidomethyl)phenyl)acetic acid (26f)

### Step-1: Preparation of ethyl 2-(2-((7-bromobenzofuran-5-yl)methoxy)-4-cyanophenyl)acetate (26b)

Compound 26b was prepared according to the procedure reported in step-4 of scheme 14 from 7-bromo-5-(bromomethyl)benzofuran (14e) (0.79 g, 2.73 mmol) using ethyl 2-(4-cyano-2-hydroxyphenyl)acetate (26a) (0.56 g, 2.73 mmol; CAS #1261647-89-5), K₂CO₃ (1.13 g, 8.19 mmol) in DMF (10 mL) and stirring at room temperature for 2h. This gave after workup and purification by flash column chromatography (silica gel (40) g, eluting with 0 to 60% EtOAc in hexane) ethyl 2-(2-((7-bromobenzofuran-5-yl)methoxy)-4-cyanophenyl)acetate (26b)(1 g, 88% yield) as a white solid; ¹H NMR (300 MHz, DMSO-d₆) δ 8.16 (d, J=2.2 Hz, 1H), 7.72 (s, 1H), 7.62-7.57 (m, 2H), 7.49-7.39 (m, 2H), 7.13 (d, J=2.3 Hz, 1H), 5.26 (s, 2H), 4.01 (q, J=7.1 Hz, 2H), 3.73 (s, 2H), 1.11-1.00 (m, 3H); MS (ES+): 436.00 & 438.00 (M+Na); MS (ES−): 412.00 & 414.00 (M−1).

### Step-2: Preparation of ethyl 2-(4-(aminomethyl)-2-((7-bromobenzofuran-5-yl)methoxy)phenyl)acetate (26c)

To a stirred solution of ethyl 2-(2-((7-bromobenzofuran-5-yl)methoxy)-4-cyanophenyl)acetate (26b) (1 g, 2.41 mmol) in anhydrous methanol (15 mL), cooled to 0° C., nickel(II) chloride hexahydrate (0.14 g, 0.60 mmol) then sodium borohydride (0.27 g, 7.24 mmol) was then added in small portions over a period of 10 min. The reaction mixture was stirred for 3 h, quenched with N1-(2-aminoethyl)ethane-1,2-diamine (0.522 mL, 4.83 mmol), stirred for 1 h and concentrated in vacuum. The residue was taken in brine (100 mL) and extracted with ethyl acetate (2×150 mL). Combined organic layer was dried, filtered and concentrated in vacuum. The residue was purified by flash column chromatography [(silica gel (40 g), eluting with MeOH/DCM from 0 to 60%)] to furnish ethyl 2-(4-(aminomethyl)-2-((7-bromobenzofuran-5-yl)methoxy)phenyl)acetate (26c) (0.3 g, 30% yield) as a pale yellow wax; MS (ES+): 418.00 & 420.05 (M+1).

### Step-3: Preparation of ethyl 2-(2-((7-bromobenzofuran-5-yl)methoxy)-4-(heptanamidomethyl)phenyl)acetate (26d)

To a solution of ethyl 2-(4-(aminomethyl)-2-((7-bromobenzofuran-5-yl)methoxy)phenyl)acetate (26c) (0.3 g, 0.72 mmol) in DCM (8 mL) was added heptanoyl chloride (0.13 g, 0.86 mmol). The reaction mixture was stirred overnight at RT and concentrated in vacuum to dryness. The residue was purified by flash column chromatography (silica gel (12g), eluting with 0-100% EtOAc/hexanes) to provide ethyl 2-(2-((7-bromobenzofuran-5-yl)methoxy)-4-(heptanamidomethyl)phenyl)acetate (26d) (0.25 g, 66% yield) as a yellow solid; MS (ES+): 530.10 & 532.20 (M+1).

### Step-4: Preparation of ethyl 2-(2-((7-(3-(aminomethyl)phenyl)benzofuran-5-yl)methoxy)-4-(heptanamidomethyl)phenyl)acetate (26e)

Compound 26e was prepared according to procedure reported in Step-4 of scheme 1 from ethyl 2-(2-((7-bromobenzofuran-5-yl)methoxy)-4-(heptanamidomethyl)phenyl)acetate (26d) (0.25 g, 0.471 mmol) in dioxane (5 mL) using 3-(aminomethyl)phenylboronic acid hydrochloride (9e) (0.088 g, 0.471 mmol), bis(triphenylphosphine)Palladium(II)chloride [Pd(PPh₃)₂Cl₂](0.050 g, 0.071 mmol), a solution of K₂CO₃ (0.195 g, 1.414 mmol) in water (1 mL) and heating at 90° C. for 3 h on oil bath. This gave after workup and purification by flash column chromatography [silica gel (12 g) eluting with DMA80 in DCM from 0-50%]ethyl 2-(2-((7-(3-(aminomethyl)phenyl)benzofuran-5-yl)methoxy)-4-(heptanamidomethyl)phenyl)acetate (26e) (0.11 g, 42% yield) as a yellow oil, which was further purified by reverse column chromatography [C18 (50 g), eluting with ACN in water (containing 0.1% HCl) from 0-100%] to give compound (26e) (0.11 g, 42% yield) HCl salt as a white solid. ¹H NMR (300 MHz, DMSO-d₆) δ 8.43 (s, 3H), 8.33 (t, J=6.0 Hz, 1H), 8.11 (d, J=2.2 Hz, 1H), 8.01 (d, J=2.0 Hz, 1H), 7.92 (dt, J=7.1, 1.8 Hz, 1H), 7.73 (d, J=1.6 Hz, 1H), 7.64-7.53 (m, 3H), 7.15 (d, J=7.7 Hz, 1H), 7.07 (d, J=2.2 Hz, 1H), 7.03 (d, J=1.6 Hz, 1H), 6.79 (dd, J=7.5, 1.5 Hz, 1H), 5.20 (s, 2H), 4.24 (d, J=5.9 Hz, 2H), 4.13 (q, J=5.8 Hz, 2H), 3.92 (q, J=7.1 Hz, 2H), 3.59 (s, 2H), 2.11 (t, J=7.4 Hz, 2H), 1.49 (q, J=7.1 Hz, 2H), 1.24 (q, J=4.9, 4.0 Hz, 6H), 0.98 (t, J=7.1 Hz, 3H), 0.87-0.77 (m, 3H); MS (ES+): 557.30 (M+1); MS (ES−): 591.30 (M+Cl).

### Step-5: Preparation of 2-(2-((7-(3-(aminomethyl)phenyl)benzofuran-5-yl)methoxy)-4-(heptanamidomethyl)phenyl)acetic acid (26f)

Compound 26f was prepared according to procedure reported in Step-8 of scheme 1 from ethyl 2-(2-((7-(3-(aminomethyl)phenyl)benzofuran-5-yl)methoxy)-4-(heptanamidomethyl)phenyl)acetate (26e) (110 mg, 0.198 mmol) in THE (4 mL), methanol (4 mL) using a solution of lithium hydroxide monohydrate (66.3 mg, 1.581 mmol) in water (1 mL) and stirring for 16 h at room temperature. This gave after workup and purification by reverse-phase column chromatography [C-18 column, eluting with 0.1% aq HCl in water and acetonitrile from 0-100%]2-(2-((7-(3-(aminomethyl)phenyl)benzofuran-5-yl)methoxy)-4-(heptanamidomethyl)phenyl)acetic acid (26f) (78 mg, 75% yield) HCl salt as a white solid; δ 8.30 (t, J=5.9 Hz, 1H), 8.11 (d, J=2.2 Hz, 1H), 8.02 (t, J=1.8 Hz, 1H), 7.94 (dt, J=7.4, 1.7 Hz, 1H), 7.75 (d, J=1.6 Hz, 1H), 7.65 (d, J=1.7 Hz, 1H), 7.63-7.50 (m, 2H), 7.14 (d, J=7.6 Hz, 1H), 7.06 (d, J=2.2 Hz, 1H), 7.01 (d, J=1.6 Hz, 1H), 6.78 (dd, J=7.6, 1.5 Hz, 1H), 5.22 (s, 2H), 4.23 (d, J=5.9 Hz, 2H), 4.13 (s, 2H), 3.55 (s, 2H), 2.10 (t, J=7.4 Hz, 2H), 1.48 (q, J=7.2 Hz, 2H), 1.24 (m, 6H), 0.88-0.76 (m, 3H); MS (ES+): 529.30 (M+1); MS (ES−): 527.25 (M−1); Analysis calculated for C₃₂H₃₆N₂O₅·HCl·H₂O: C, 65.91; H, 6.74; Cl, 6.08; N, 4.80; Found: C, 66.12; H, 6.46; Cl, 5.83; N, 4.87.

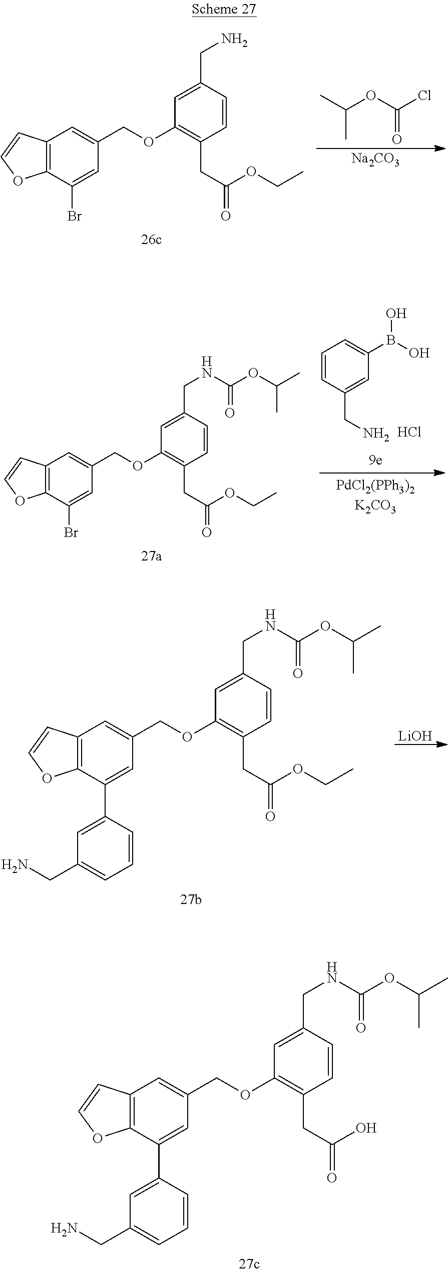

### Preparation of 2-(2-((7-(3-(aminomethyl)phenyl)benzofuran-5-yl)methoxy)-4-((isopropoxycarbonylamino)methyl)phenyl)acetic acid (27c)

### Step-1: Preparation of ethyl 2-(2-((7-bromobenzofuran-5-yl)methoxy)-4-((isopropoxycarbonylamino)methyl)phenyl)acetate (27a)

Compound 27a was prepared according to the procedure reported in step-6 of scheme 25 from ethyl 2-(4-(aminomethyl)-2-((7-bromobenzofuran-5-yl)methoxy)phenyl)acetate (26c) (400 mg, 0.96 mmol) in THF/H₂O (1:1, 8 mL) using NaHCO₃ (121 mg, 1.43 mmol), a solution of isopropyl carbonochloridate (1.43 mL, 1.43 mmol) in toluene and stirring for 1 h at RT. This gave after workup and purification by flash column chromatography [silica gel column (12 g), eluting with 0-80% EtOAc/hexanes]ethyl 2-(2-((7-bromobenzofuran-5-yl)methoxy)-4-((isopropoxycarbonylamino)methyl)phenyl)acetate (27a) (375 mg, 78% yield) as a colorless oil; ¹H NMR (300 MHz, DMSO-d₆) δ 8.14 (d, J=2.1 Hz, 1H), 7.71 (d, J=1.5 Hz, 1H), 7.60 (d, J=1.5 Hz, 1H), 7.55 (t, J=6.2 Hz, 1H), 7.16 (d, J=7.6 Hz, 1H), 7.11 (d, J=2.2 Hz, 1H), 7.02-6.95 (m, 1H), 6.80 (dd, J=7.5, 1.5 Hz, 1H), 5.14 (s, 2H), 4.74 (h, J=6.2 Hz, 1H), 4.15 (d, J=6.2 Hz, 2H), 4.00 (d, J=7.1 Hz, 2H), 3.58 (s, 2H), 1.16 (d, J=6.6 Hz, 6H), 1.07 (t, J=7.1 Hz, 3H); MS (ES+): 526.10 & 528.10 (M+Na).

### Step-2: Preparation of ethyl 2-(2-((7-(3-(aminomethyl)phenyl)benzofuran-5-yl)methoxy)-4-((isopropoxycarbonylamino)methyl)phenyl)acetate (27b)

Compound 27b was prepared according to procedure reported in Step-4 of scheme 1 from ethyl 2-(2-((7-bromobenzofuran-5-yl)methoxy)-4-((isopropoxycarbonylamino)methyl)phenyl)acetate (27a) (375 mg, 0.74 mmol) in dioxane (5 mL) using 3-(aminomethyl)phenylboronic acid hydrochloride (9e) (209 mg, 1.12 mmol), bis(triphenylphosphine)Palladium(II)chloride [Pd(PPh₃)₂Cl₂](78 mg, 0.11 mmol), a solution of K₂CO₃ (308 mg, 2.23 mmol) in water (1 mL) and heating at 90° C. for 3 h on oil bath. This gave after workup and purification by flash column chromatography [silica gel (12 g), eluting with DMA-80 in DCM from 0-50%]ethyl 2-(2-((7-(3-(aminomethyl)phenyl)benzofuran-5-yl)methoxy)-4-((isopropoxycarbonylamino)methyl)phenyl)acetate (27b) (240 mg, 61% yield) as a light-yellow oil. ¹H NMR (300 MHz, DMSO-d₆) δ 8.51 (s, 3H), 8.11 (d, J=2.2 Hz, 1H), 8.01 (t, J=1.6 Hz, 1H), 7.92 (m, 1H), 7.73 (d, J=1.6 Hz, 1H), 7.62 (d, J=1.6 Hz, 1H), 7.58 (td, J=4.6, 4.1, 2.8 Hz, 3H), 7.16 (d, J=7.6 Hz, 1H), 7.07 (d, J=2.2 Hz, 1H), 7.05 (d, J=1.5 Hz, 1H), 6.80 (dd, J=7.7, 1.4 Hz, 1H), 5.21 (s, 2H), 4.75 (hept, J=6.2 Hz, 1H), 4.22-4.05 (m, 4H), 3.92 (q, J=7.1 Hz, 2H), 3.59 (s, 2H), 1.16 (d, J=6.2 Hz, 6H), 0.98 (t, J=7.1 Hz, 3H); MS (ES+): 531.30 (M+1).

### Step-3: Preparation of 2-(2-((7-(3-(aminomethyl)phenyl)benzofuran-5-yl)methoxy)-4-((isopropoxycarbonylamino)methyl)phenyl)acetic acid (27c)

Compound 27c was prepared according to procedure reported in Step-8 of scheme 1 from ethyl 2-(2-((7-(3-(aminomethyl)phenyl)benzofuran-5-yl)methoxy)-4-((isopropoxycarbonylamino)methyl)phenyl)acetate (27b) (150 mg, 0.28 mmol) in THE (4 mL), methanol (4 mL) using a solution of lithium hydroxide monohydrate (95 mg, 2.26 mmol) in water (1 mL) and stirring for 5 h at room temperature. This gave after workup and purification by reverse-phase column chromatography [C-18 column (50 g), eluting with 0.1% aq HCl in water and acetonitrile from 0-100%]2-(2-((7-(3-(aminomethyl)phenyl)benzofuran-5-yl)methoxy)-4-((isopropoxycarbonylamino)methyl)phenyl)acetic acid (27c) (115 mg, 81% yield) HCl salt as a white solid; ¹H NMR (300 MHz, DMSO-d₆) δ 8.37 (s, 2H), 8.11 (d, J=2.2 Hz, 1H), 8.00 (d, J=1.8 Hz, 1H), 7.94 (dt, J=7.5, 1.7 Hz, 1H), 7.76 (d, J=1.6 Hz, 1H), 7.65 (d, J=1.6 Hz, 1H), 7.63-7.51 (m, 3H), 7.15 (d, J=7.6 Hz, 1H), 7.06 (d, J=2.2 Hz, 1H), 7.02 (d, J=1.6 Hz, 1H), 6.79 (dd, J=7.7, 1.5 Hz, 1H), 5.23 (s, 2H), 4.74 (p, J=6.2 Hz, 1H), 4.18-4.10 (m, 4H), 3.55 (s, 2H), 1.15 (d, J=6.2 Hz, 6H); MS (ES+): 503.20 (M+1); MS (ES−): 501.20 (M−1); Analysis calculated for C₂₉H₃₀N₂O₆·HCl·H₂O: C, 62.53; H, 5.97; Cl, 6.36; N, 5.03; Found: C, 62.77; H, 6.18; Cl, 6.44; N, 5.16.

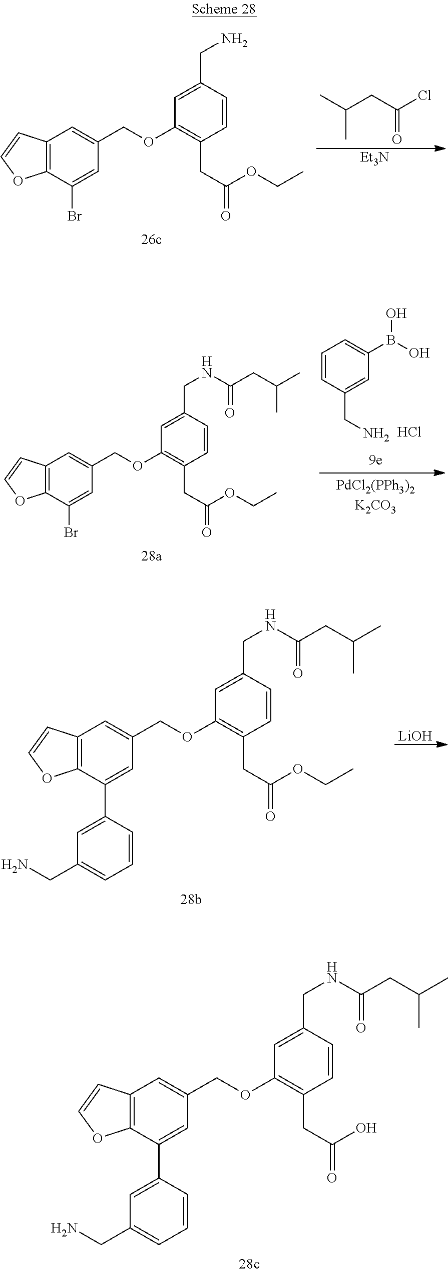

### Preparation of 2-(2-((7-(3-(aminomethyl)phenyl)benzofuran-5-yl)methoxy)-4-((3-methylbutanamido)methyl)phenyl)acetic acid (28c)

### Step-1: Preparation of ethyl 2-(2-((7-bromobenzofuran-5-yl)methoxy)-4-((3-methylbutanamido)methyl)phenyl)acetate (28a)

Compound 28a was prepared according to the procedure reported in step-6 of scheme 25 from ethyl 2-(4-(aminomethyl)-2-((7-bromobenzofuran-5-yl)methoxy)phenyl)acetate (26c) (300 mg, 0.72 mmol) in DCM (4 mL) using triethylamine (0.15 mL, 1.08 mmol), 3-methylbutanoyl chloride (130 mg, 1.076 mmol) and stirring for 1 h at RT. This gave after workup and purification by flash column chromatography [silica gel column (12 g), eluting with 0-80% EtOAc/hexanes]ethyl 2-(2-((7-bromobenzofuran-5-yl)methoxy)-4-((3-methylbutanamido)methyl)phenyl)acetate (28a) (300 mg, 83% yield) as a white solid; ¹H NMR (300 MHz, DMSO-d₆) δ 8.28 (t, J=5.9 Hz, 1H), 8.15 (d, J=2.2 Hz, 1H), 7.70 (d, J=1.5 Hz, 1H), 7.59 (d, J=1.5 Hz, 1H), 7.15 (dd, J=7.6, 2.9 Hz, 1H), 7.11 (d, J=2.2 Hz, 1H), 6.96 (d, J=1.6 Hz, 1H), 6.80 (dd, J=7.6, 1.5 Hz, 1H), 5.13 (d, J=4.0 Hz, 2H), 4.24 (d, J=5.9 Hz, 2H), 4.00 (q, J=7.1 Hz, 2H), 3.61 (s, 2H), 2.04-1.96 (m, 3H), 1.07 (t, J=7.1 Hz, 3H), 0.89-0.85 (m, 6H); MS (ES+): 502.10 & 504.10 (M+1); 524.10 & 526.10 (M+Na); MS (ES−): 536.10 & 538.10 (M+Cl).

### Step-2: Preparation of ethyl 2-(2-((7-(3-(aminomethyl)phenyl)benzofuran-5-yl)methoxy)-4-((3-methylbutanamido)methyl)phenyl)acetate (28b)

Compound 28b was prepared according to procedure reported in Step-4 of scheme 1 from ethyl 2-(2-((7-bromobenzofuran-5-yl)methoxy)-4-((3-methylbutanamido)methyl)phenyl)acetate (28a) (270 mg, 0.54 mmol) in dioxane (5 mL) using 3-(aminomethyl)phenylboronic acid hydrochloride (9e) (151 mg, 0.81 mmol), bis(triphenylphosphine)Palladium(II)chloride [Pd(PPh₃)₂Cl₂](56.6 mg, 0.08 mmol), a solution of K₂CO₃ (223 mg, 1.61 mmol) in water (1 mL) and heating at 90° C. for 3 h on oil bath. This gave after workup and purification by flash column chromatography [silica gel (12 g) eluting with DMA-80 in DCM from 0-50%]ethyl 2-(2-((7-(3-(aminomethyl)phenyl)benzofuran-5-yl)methoxy)-4-((3-methylbutanamido)methyl)phenyl)acetate (28b) (0.2 g, 70% yield) as a light-yellow oil. ¹H NMR (300 MHz, DMSO-d₆) δ 8.48 (s, 2H), 8.36 (m, 1H), 8.12 (d, J=2.2 Hz, 1H), 8.01 (s, 1H), 7.92 (dt, J=6.9, 2.0 Hz, 1H), 7.72 (d, J=1.6 Hz, 1H), 7.64-7.53 (m, 3H), 7.16 (d, J=7.6 Hz, 1H), 7.07 (d, J=2.2 Hz, 1H), 7.03 (d, J=1.6 Hz, 1H), 6.80 (dd, J=7.5, 1.5 Hz, 1H), 5.20 (s, 2H), 4.25 (d, J=5.9 Hz, 2H), 4.13 (d, J=5.7 Hz, 2H), 3.92 (q, J=7.1 Hz, 2H), 3.62 (s, 2H), 2.04-1.94 (m, 3H), 0.98 (t, J=7.1 Hz, 3H), 0.91-0.81 (m, 6H); MS (ES+): 529.30 (M+1); MS (ES−): 563.30 (M+Cl).

### Step-3: Preparation of 2-(2-((7-(3-(aminomethyl)phenyl)benzofuran-5-yl)methoxy)-4-((3-methylbutanamido)methyl)phenyl)acetic acid (28c)

Compound 28c was prepared according to procedure reported in Step-8 of scheme 1 from ethyl 2-(2-((7-(3-(aminomethyl)phenyl)benzofuran-5-yl)methoxy)-4-((3-methylbutanamido)methyl)phenyl)acetate (28b) (100 mg, 0.19 mmol) in THE (4 mL), methanol (4 mL) using a solution of lithium hydroxide monohydrate (63.5 mg, 1.51 mmol) in water (1 mL) and stirring for 5 h at room temperature. This gave after workup and purification by reverse-phase column chromatography [C-18 column (50 g), eluting with 0.1% aq HCl in water and acetonitrile from 0-100%]2-(2-((7-(3-(aminomethyl)phenyl)benzofuran-5-yl)methoxy)-4-((3-methylbutanamido)methyl)phenyl)acetic acid (28c) (75 mg, 79% yield) HCl salt as a white solid; ¹H NMR (300 MHz, DMSO-d₆) δ 8.31 (br s, 3H, D₂O exchangeable), 8.11 (d, J=2.2 Hz, 1H), 8.00 (d, J=1.8 Hz, 1H), 7.94 (dt, J=7.5, 1.6 Hz, 1H), 7.75 (d, J=1.6 Hz, 1H), 7.63 (t, J=1.7 Hz, 1H), 7.59 (d, J=7.6 Hz, 1H), 7.54 (dt, J=7.6, 1.6 Hz, 1H), 7.15 (d, J=7.6 Hz, 1H), 7.06 (d, J=2.2 Hz, 1H), 7.01 (d, J=1.5 Hz, 1H), 6.79 (dd, J=7.6, 1.5 Hz, 1H), 5.22 (s, 2H), 4.24 (d, J=5.9 Hz, 2H), 4.14 (s, 2H), 3.56 (s, 2H), 2.05-1.91 (m, 3H), 0.90-0.82 (m, 6H); MS (ES+): 501.20 (M+1); MS (ES−): 999.20 (2M−1); Analysis Calculated for: C₃₀H₃₂N₂O₅·1.05HCl·1.25H₂O. C, 64.18; H, 6.38; Cl, 6.63; N, 4.99; C, 64.34; H, 6.31; Cl, 6.84; N, 5.08.

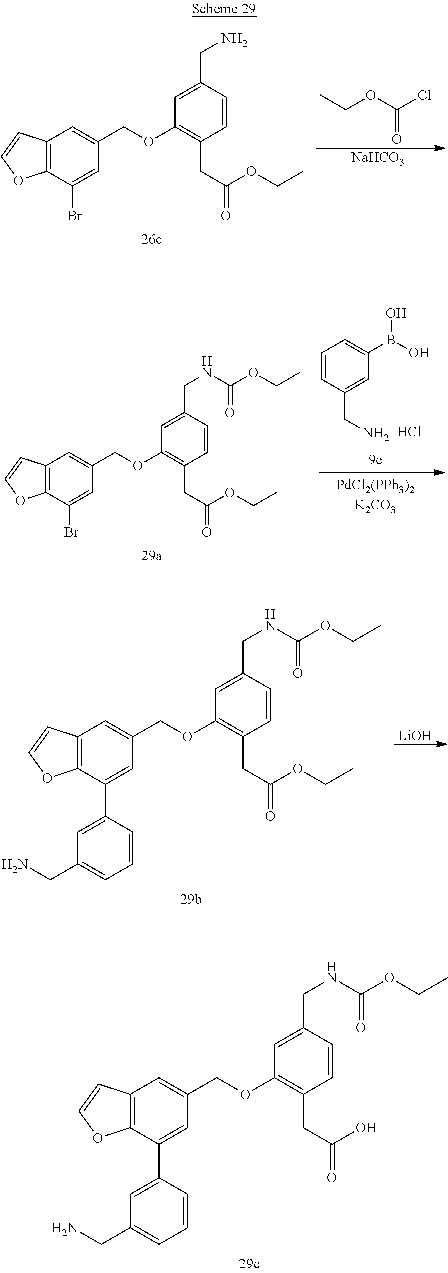

### Preparation of 2-(2-((7-(3-(aminomethyl)phenyl)benzofuran-5-yl)methoxy)-4-((ethoxycarbonylamino)methyl)phenyl)acetic acid (29c)

### Step-1: Preparation of ethyl 2-(2-((7-bromobenzofuran-5-yl)methoxy)-4-((ethoxycarbonylamino)methyl)phenyl)acetate (29a)

Compound 29a was prepared according to the procedure reported in step-6 of scheme 25 from ethyl 2-(4-(aminomethyl)-2-((7-bromobenzofuran-5-yl)methoxy)phenyl)acetate (26c) (400 mg, 0.96 mmol) in THF/H₂O (1:1, 8 mL) using NaHCO₃ (121 mg, 1.43 mmol), a solution of ethyl carbonochloridate (156 mg, 1.43 mmol) and stirring for 1 h at RT. This gave after workup and purification by flash column chromatography [silica gel column (12 g), eluting with 0-80% EtOAc/hexanes]ethyl 2-(2-((7-bromobenzofuran-5-yl)methoxy)-4-((ethoxycarbonylamino)methyl)phenyl)acetate (29a) (300 mg, 64% yield) as a colorless oil; H NMR (300 MHz, DMSO-d₆) δ 8.15 (d, J=2.2 Hz, 1H), 7.71 (d, J=1.5 Hz, 1H), 7.66-7.58 (m, 2H), 7.15 (dd, J=7.6, 3.1 Hz, 1H), 7.11 (d, J=2.2 Hz, 1H), 6.99 (d, J=1.5 Hz, 1H), 6.80 (dd, J=7.5, 1.4 Hz, 1H), 5.14 (s, 2H), 4.17-4.14 (m, 2H), 4.01-3.98 (m, 2H), 3.58 (d, J=3.6 Hz, 2H), 3.17 (d, J=5.2 Hz, 2H), 1.16 (t, J=7.1 Hz, 3H), 1.06 (t, J=7.1 Hz, 3H); MS (ES+): 512.05 & 514.10 (M+Na).

### Step-2: Preparation of ethyl 2-(2-((7-(3-(aminomethyl)phenyl)benzofuran-5-yl)methoxy)-4-((ethoxycarbonylamino)methyl)phenyl)acetate (29b)

Compound 29b was prepared according to procedure reported in Step-4 of scheme 1 from ethyl 2-(2-((7-bromobenzofuran-5-yl)methoxy)-4-((ethoxycarbonylamino)methyl)phenyl)acetate (29a) (300 mg, 0.612 mmol) in dioxane (5 mL) using 3-(aminomethyl)phenylboronic acid hydrochloride (9e) (172 mg, 0.918 mmol), bis(triphenylphosphine)Palladium(II)chloride [Pd(PPh₃)₂Cl₂](64.4 mg, 0.092 mmol), a solution of K₂CO₃ (254 mg, 1.835 mmol) in water (1 mL) and heating at 90° C. for 3 h on oil bath. This gave after workup and purification by flash column chromatography [silica gel (12 g) eluting with DMA-80 in DCM from 0-50%]ethyl 2-(2-((7-(3-(aminomethyl)phenyl)benzofuran-5-yl)methoxy)-4-((ethoxycarbonylamino)methyl)phenyl)acetate (29b) (0.2 g, 63% yield) as a light yellow oil. The submitted sample was further purified by reverse column [C18 (50 g), eluting with ACN in water (containing 0.1% HCl) from 0-100%] to give 55 mg desired compound as a white solid; ¹H NMR (300 MHz, DMSO-d₆) δ 8.33 (s, 3H), 8.12 (d, J=2.2 Hz, 1H), 7.99 (s, 1H), 7.92 (dt, J=7.6, 1.6 Hz, 1H), 7.74 (d, J=1.6 Hz, 1H), 7.66 (t, J=7.2 Hz, 1H), 7.59 (d, J=7.5 Hz, 2H), 7.54 (d, J=7.9 Hz, 1H), 7.16 (d, J=7.6 Hz, 1H), 7.08 (d, J=2.2 Hz, 1H), 7.05 (s, 1H), 6.80 (dd, J=7.6, 1.5 Hz, 1H), 5.21 (s, 2H), 4.23-4.09 (m, 4H), 4.06-3.84 (m, 4H), 3.59 (s, 2H), 1.15 (t, J=7.1 Hz, 3H), 0.97 (t, J=7.1 Hz, 3H); MS (ES+): 517.20 (M+1).

### Step-3: Preparation of 2-(2-((7-(3-(aminomethyl)phenyl)benzofuran-5-yl)methoxy)-4-((ethoxycarbonylamino)methyl)phenyl)acetic acid (29c)

Compound 29c was prepared according to procedure reported in Step-8 of scheme 1 from ethyl 2-(2-((7-(3-(aminomethyl)phenyl)benzofuran-5-yl)methoxy)-4-((ethoxycarbonylamino)methyl)phenyl)acetate (29b) (120 mg, 0.23 mmol) in THE (4 mL), methanol (4 mL) using a solution of lithium hydroxide monohydrate (78 mg, 1.86 mmol) in water (1 mL) and stirring for 5 h at room temperature. This gave after workup and purification by reverse-phase column chromatography [C-18 column (50 g), eluting with 0.1% aq HCl in water and acetonitrile from 0-100%]2-(2-((7-(3-(aminomethyl)phenyl)benzofuran-5-yl)methoxy)-4-((ethoxycarbonylamino)methyl)phenyl)acetic acid (29c) (95 mg, 84% yield) HCl salt as a white solid; ¹H NMR (300 MHz, DMSO-d₆) δ 8.46 (s, 3H, D₂O exchangeable), 8.11 (d, J=2.2 Hz, 1H), 8.01 (d, J=1.8 Hz, 1H), 7.94 (dt, J=7.3, 1.8 Hz, 1H), 7.76 (d, J=1.6 Hz, 1H), 7.69-7.60 (m, 2H), 7.60-7.52 (m, 2H), 7.15 (d, J=7.6 Hz, 1H), 7.06 (d, J=2.2 Hz, 1H), 7.05-6.98 (m, 1H), 6.79 (dd, J=7.6, 1.5 Hz, 1H), 5.23 (s, 2H), 4.20-4.08 (m, 4H), 3.98 (q, J=7.1 Hz, 2H), 3.56 (s, 2H), 1.14 (t, J=7.1 Hz, 3H); MS (ES+): 489.20 (M+1); Analysis Calculated for: C₂₈H₂₈N₂O₆·HCl·H₂O. C, 61.93; H, 5.75; Cl, 6.53; N, 5.16; Found; C, 61.52; H, 5.77; Cl, 6.87; N, 5.23.

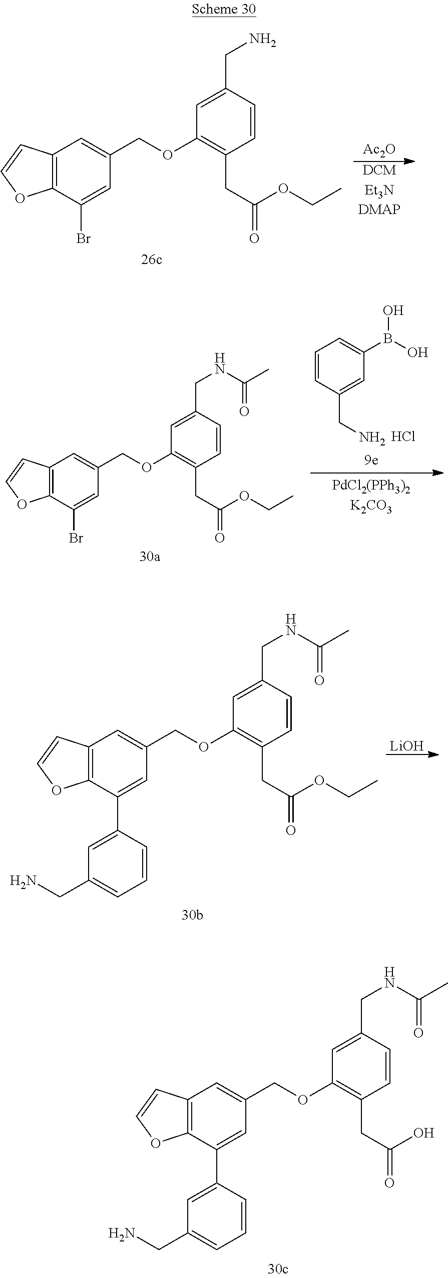

### Preparation of 2-(4-(acetamidomethyl)-2-((7-(3-(aminomethyl)phenyl)benzofuran-5-yl)methoxy)phenyl)acetic acid (30c)

### Step-1: Preparation of ethyl 2-(4-(acetamidomethyl)-2-((7-bromobenzofuran-5-yl)methoxy)phenyl)acetate (30a)

Compound 30a was prepared according to the procedure reported in step-6 of scheme 25 from ethyl 2-(4-(aminomethyl)-2-((7-bromobenzofuran-5-yl)methoxy)phenyl)acetate (26c) (300 mg, 0.72 mmol) in DCM (4 mL) using NEt₃ (0.20 mL, 1.43 mmol), acetic anhydride (0.14 mL, 1.43 mmol) and stirring for 1 h at RT. This gave after workup and purification by flash column chromatography [silica gel column (12 g), eluting with 0-80% EtOAc/hexanes]ethyl 2-(4-(acetamidomethyl)-2-((7-bromobenzofuran-5-yl)methoxy)phenyl)acetate (30a) (280 mg, 85% yield) as a colorless oil; ¹H NMR (300 MHz, DMSO-d₆) δ 8.32 (t, J=5.9 Hz, 1H), 8.15 (d, J=2.2 Hz, 1H), 7.72 (d, J=1.5 Hz, 1H), 7.60 (d, J=1.6 Hz, 1H), 7.15 (dd, J=7.6, 2.9 Hz, 1H), 7.11 (d, J=2.2 Hz, 1H), 6.98 (t, J=1.8 Hz, 1H), 6.83-6.77 (m, 1H), 5.14 (d, J=3.1 Hz, 2H), 4.22 (d, J=5.9 Hz, 2H), 4.01 (q, J=7.0 Hz, 2H), 3.59-3.58 (m, 2H), 1.86 (s, 3H), 1.07 (t, J=7.1 Hz, 3H); MS (ES+): 460.05 & 462.00 (M+1); 482.05 & 484.00 (M+Na); MS (ES−): 494.00 & 496.00 (M+Cl).

### Step-2: Preparation of ethyl 2-(4-(acetamidomethyl)-2-((7-(3-(aminomethyl)phenyl)benzofuran-5-yl)methoxy)phenyl)acetate (30b)

Compound 30b was prepared according to procedure reported in Step-4 of scheme 1 from ethyl 2-(4-(acetamidomethyl)-2-((7-bromobenzofuran-5-yl)methoxy)phenyl)acetate (30a) (280 mg, 0.608 mmol) in dioxane (5 mL) using 3-(aminomethyl)phenylboronic acid hydrochloride (9e) (171 mg, 0.912 mmol), bis(triphenylphosphine)Palladium(II)chloride [Pd(PPh₃)₂Cl₂](64.0 mg, 0.091 mmol), a solution of K₂CO₃ (252 mg, 1.825 mmol) in water (1 mL) and heating at 90° C. for 3 h on oil bath. This gave after workup and purification by flash column chromatography [silica gel (12 g) eluting with DMA-80 in DCM from 0-50%]ethyl 2-(4-(acetamidomethyl)-2-((7-(3-(aminomethyl)phenyl)benzofuran-5-yl)methoxy)phenyl)acetate (30b) (70 mg, 23% yield) as a white solid. ¹H NMR (300 MHz, DMSO-d₆) δ 8.46 (s, 2H), 8.40 (t, J=5.9 Hz, 1H), 8.11 (d, J=2.2 Hz, 1H), 8.01 (d, J=2.0 Hz, 1H), 7.92 (dt, J=7.0, 2.0 Hz, 1H), 7.74 (d, J=1.6 Hz, 1H), 7.62 (d, J=1.7 Hz, 1H), 7.61-7.53 (m, 2H), 7.15 (d, J=7.6 Hz, 1H), 7.08 (d, J=2.2 Hz, 1H), 7.05 (d, J=1.6 Hz, 1H), 6.83-6.75 (m, 1H), 5.22 (s, 2H), 4.23 (d, J=5.9 Hz, 2H), 4.13 (q, J=5.8 Hz, 2H), 3.92 (q, J=7.1 Hz, 2H), 3.59 (s, 2H), 1.85 (s, 3H), 0.98 (t, J=7.1 Hz, 3H); MS (ES+): 487.20 (M+1); MS (ES−): 521.50 (M+Cl); LC, 1.99 min, 99.80%.

### Step-3: Preparation of 2-(4-(acetamidomethyl)-2-((7-(3-(aminomethyl)phenyl)benzofuran-5-yl)methoxy)phenyl)acetic acid (30c)

Compound 30c was prepared according to procedure reported in Step-8 of scheme 1 from ethyl 2-(4-(acetamidomethyl)-2-((7-(3-(aminomethyl)phenyl)benzofuran-5-yl)methoxy)phenyl)acetate (30b) (120 mg, 0.25 mmol) in THE (4 mL), methanol (4 mL) using a solution of lithium hydroxide monohydrate (83 mg, 1.97 mmol) in water (1 mL) and stirring for 5 h at room temperature. This gave after workup and purification by reverse-phase column chromatography [C-18 column (50 g), eluting with 0.1% aq HCl in water and acetonitrile from 0-100%]2-(4-(acetamidomethyl)-2-((7-(3-(aminomethyl)phenyl)benzofuran-5-yl)methoxy)phenyl)acetic acid (30c) (95 mg, 84% yield) HCl salt as a white solid; ¹H NMR (300 MHz, DMSO-d₆) δ 12.21 (s, 1H, D₂O exchangeable), 8.45-8.23 (m, 4H, D₂O exchangeable), 8.11 (d, J=2.2 Hz, 1H), 8.00 (d, J=1.7 Hz, 1H), 7.94 (dt, J=7.5, 1.7 Hz, 1H), 7.77 (d, J=1.6 Hz, 1H), 7.65 (d, J=1.7 Hz, 1H), 7.60 (t, J=7.6 Hz, 1H), 7.54 (dt, J=7.7, 1.6 Hz, 1H), 7.15 (d, J=7.6 Hz, 1H), 7.06 (d, J=2.2 Hz, 1H), 7.02 (d, J=1.5 Hz, 1H), 6.79 (dd, J=7.6, 1.5 Hz, 1H), 5.24 (s, 2H), 4.22 (d, J=5.8 Hz, 2H), 4.14 (q, J=5.8 Hz, 2H), 3.56 (s, 2H), 1.84 (s, 3H); MS (ES+): 459.10 (M+1); Analysis Calculated for: C₂₇H₂₆N₂O₅·HCl·1.75H₂O. C, 61.60; H, 5.84; Cl, 6.73; N, 5.32; found: C, 61.56; H, 5.72; Cl, 6.95; N, 5.35.

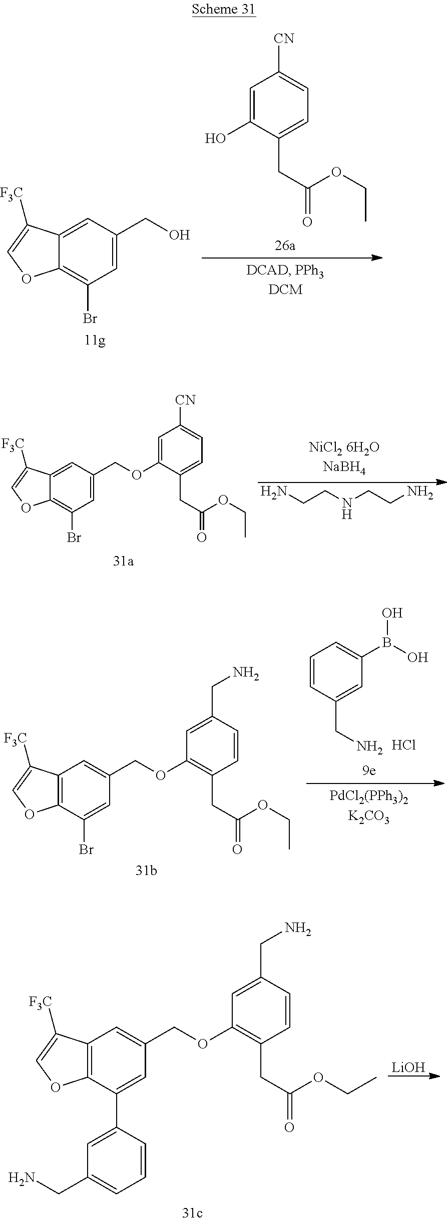

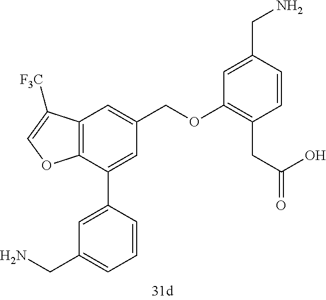 [SMILES: NCc1cccc(-c2cc(COc3cc(CN)ccc3CC(=O)O)cc3c(C(F)(F)F)coc23)c1]

### Preparation of 2-(4-(aminomethyl)-2-((7-(3-(aminomethyl)phenyl)-3-(trifluoromethyl)benzofuran-5-yl)methoxy)phenyl)acetic acid (31d)

### Step-1: Preparation of ethyl 2-(2-((7-bromo-3-(trifluoromethyl)benzofuran-5-yl)methoxy)-4-cyanophenyl)acetate (31a)

Compound 31a was prepared according to procedure reported in Step-2 of scheme 1 from (7-bromo-3-(trifluoromethyl)benzofuran-5-yl)methanol (11g) (0.8 g, 2.71 mmol) in DCM (30 mL) using triphenylphosphine (0.78 g, 2.98 mmol), ethyl 2-(4-cyano-2-hydroxyphenyl)acetate (26a) (0.61 g, 2.98 mmol), a solution of (E)-bis(4-chlorobenzyl) diazene-1,2-dicarboxylate (DCAD) (1.10 g, 2.98 mmol) in DCM (20 mL) and stirring at room temperature for 30 min. This gave after workup and purification by flash column chromatography (silica gel (40 g), eluting with 0 to 50% ethyl acetate in hexanes) ethyl 2-(2-((7-bromo-3-(trifluoromethyl)benzofuran-5-yl)methoxy)-4-cyanophenyl)acetate (31a) (0.4 g, 31% yield) as a clear oil; ¹H NMR (300 MHz, DMSO-d₆) δ 8.99 (q, J=1.7 Hz, 1H), 7.89-7.74 (m, 2H), 7.60 (d, J=1.3 Hz, 1H), 7.49-7.39 (m, 2H), 5.33 (s, 2H), 4.04-3.96 (m, 2H), 3.70 (s, 2H), 1.05 (t, J=7.1 Hz, 3H); MS (ES−): 480.40 (M−1).

### Step-2: Preparation of ethyl 2-(4-(aminomethyl)-2-((7-bromo-3-(trifluoromethyl)benzofuran-5-yl)methoxy)phenyl)acetate (31b)

Compound 31b was prepared according to procedure reported in Step-2 of scheme 26 from ethyl 2-(2-((7-bromo-3-(trifluoromethyl)benzofuran-5-yl)methoxy)-4-cyanophenyl)acetate (31a) (0.38 g, 0.79 mmol) in anhydrous methanol (10 mL), using nickel(II) chloride hexahydrate (0.05 g, 0.20 mmol), sodium borohydride (0.089 g, 2.364 mmol) and N1-(2-aminoethyl)ethane-1,2-diamine (0.17 mL, 1.58 mmol) for quenching. This gave after workup and purification by flash column chromatography [(silica gel (40 g), eluting with MeOH/DCM from 0 to 60%)]ethyl 2-(4-(aminomethyl)-2-((7-bromo-3-(trifluoromethyl)benzofuran-5-yl)methoxy)phenyl)acetate (31b) (50 mg, 13% yield) as a pale yellow wax; MS (ES+): 486.00 & 488.00 (M+1).

### Step-3: Preparation of ethyl 2-(4-(aminomethyl)-2-((7-(3-(aminomethyl)phenyl)-3-(trifluoromethyl)benzofuran-5-yl)methoxy)phenyl)acetate (31c)

Compound 31c was prepared according to procedure reported in Step-4 of scheme 1 from ethyl 2-(4-(aminomethyl)-2-((7-bromo-3-(trifluoromethyl)benzofuran-5-yl)methoxy)phenyl)acetate (31b) (50 mg, 0.103 mmol) in dioxane (3 mL) using 3-(aminomethyl)phenylboronic acid hydrochloride (9e) (38.5 mg, 0.206 mmol), bis(triphenylphosphine)Palladium(II)chloride [Pd(PPh₃)₂Cl₂](10.83 mg, 0.015 mmol), a solution of K₂CO₃ (42.6 mg, 0.308 mmol) in water (1 mL) and heating at 90° C. for 3 h on oil bath. This gave after workup and purification by flash column chromatography [silica gel (12 g), eluting with DMA80 in DCM from 0-50%]ethyl 2-(4-(aminomethyl)-2-((7-(3-(aminomethyl)phenyl)-3-(trifluoromethyl)benzofuran-5-yl)methoxy)phenyl)acetate (31c) (30 mg, 57% yield) as a light yellow oil; MS (ES+): 513.20 (M+1).

### Step-4: Preparation of 2-(4-(aminomethyl)-2-((7-(3-(aminomethyl)phenyl)-3-(trifluoromethyl)benzofuran-5-yl)methoxy)phenyl)acetic acid (31d)

Compound 31d was prepared according to procedure reported in Step-8 of scheme 1 from ethyl 2-(4-(aminomethyl)-2-((7-(3-(aminomethyl)phenyl)-3-(trifluoromethyl)benzofuran-5-yl)methoxy)phenyl)acetate (31c) (30 mg, 0.059 mmol) in THE (4 mL), methanol (4 mL) using a solution of lithium hydroxide monohydrate (19.65 mg, 0.468 mmol) in water (1 mL) and stirring for 16 h at room temperature. This gave after workup and purification by reverse-phase column chromatography [C-18 column, eluting with 0.1% aq HCl in water and acetonitrile from 0-100%]2-(4-(aminomethyl)-2-((7-(3-(aminomethyl)phenyl)-3-(trifluoromethyl)benzofuran-5-yl)methoxy)phenyl)acetic acid (31d) (19 mg, 67% yield) HCl salt as a light yellow solid; ¹H NMR (300 MHz, DMSO-d₆) δ 12.25 (s, 1H, D₂O exchangeable), 8.93 (t, J=1.8 Hz, 1H), 8.44 (s, 6H), 8.03 (d, J=2.1 Hz, 1H), 7.92 (dt, J=6.6, 2.2 Hz, 1H), 7.87-7.81 (m, 2H), 7.64-7.57 (m, 2H), 7.40 (d, J=1.6 Hz, 1H), 7.26 (d, J=7.6 Hz, 1H), 7.03 (dd, J=7.6, 1.5 Hz, 1H), 5.33 (s, 2H), 4.15 (s, 2H), 4.00 (s, 2H), 3.61 (s, 2H); ¹⁹F NMR (282 MHz, DMSO-d₆) δ −57.96; MS (ES+): 485.10 (M+1).

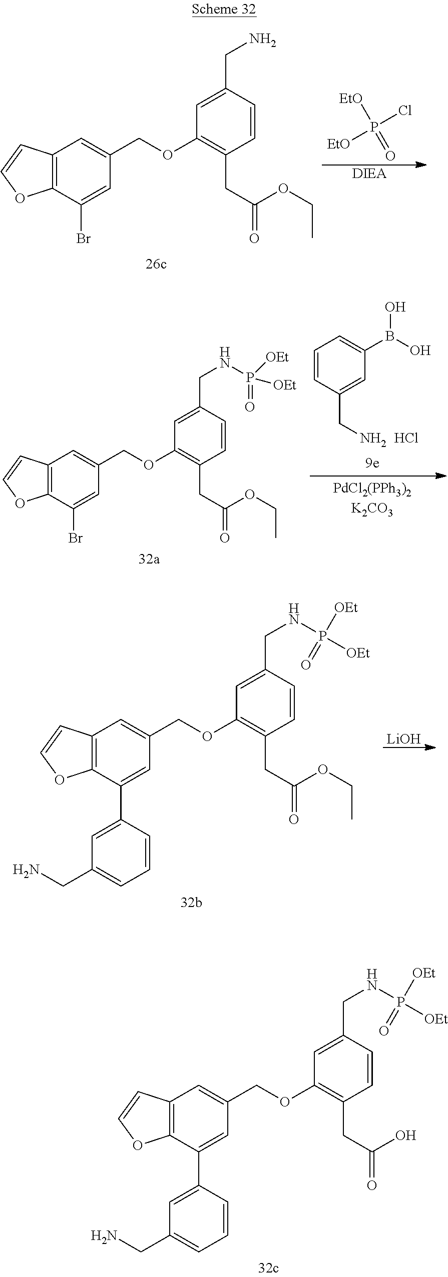

### Preparation of 2-(2-((7-(3-(aminomethyl)phenyl)benzofuran-5-yl)methoxy)-4-((diethoxyphosphorylamino)methyl)phenyl)acetic acid (32c)

### Step-1: Preparation of ethyl 2-(2-((7-bromobenzofuran-5-yl)methoxy)-4-((diethoxyphosphorylamino)methyl)phenyl)acetate (32a)

Compound 32a was prepared according to the procedure reported in step-6 of scheme 25 from ethyl 2-(4-(aminomethyl)-2-((7-bromobenzofuran-5-yl)methoxy)phenyl)acetate (26c) (300 mg, 0.72 mmol) in DCM (4 mL) using N-ethyl-N-isopropylpropan-2-amine (0.25 mL, 1.43 mmol), diethyl phosphorochloridate (0.21 mL, 1.43 mmol) and stirring for 1 h at RT. This gave after workup and purification by flash column chromatography [silica gel column (24 g), eluting with 0-50% EtOAc/hexanes]ethyl 2-(2-((7-bromobenzofuran-5-yl)methoxy)-4-((diethoxyphosphorylamino)methyl)phenyl)acetate (32a) (35 mg, 9% yield) as a clear oil; MS (ES+): 554.10 & 556.05 (M+1).

### Step-2: Preparation of ethyl 2-(2-((7-(3-(aminomethyl)phenyl)benzofuran-5-yl)methoxy)-4-((diethoxyphosphorylamino)methyl)phenyl)acetate (32b)

Compound 32b was prepared according to procedure reported in Step-4 of scheme 1 from ethyl 2-(2-((7-bromobenzofuran-5-yl)methoxy)-4-((diethoxyphosphorylamino)methyl)phenyl)acetate (32a) (70 mg, 0.13 mmol) in dioxane (4 mL) using 3-(aminomethyl)phenylboronic acid hydrochloride (9e) (47.3 mg, 0.25 mmol), bis(triphenylphosphine)Palladium(II)chloride [Pd(PPh₃)₂Cl₂](13.29 mg, 0.02 mmol), a solution of K₂CO₃ (52.4 mg, 0.38 mmol) in water (1 mL) and heating at 90° C. for 3 h on oil bath. This gave after workup and purification by flash column chromatography [silica gel (12 g), eluting with DMA-80 in DCM from 0-50%]ethyl 2-(2-((7-(3-(aminomethyl)phenyl)benzofuran-5-yl)methoxy)-4-((diethoxyphosphorylamino)methyl)phenyl)acetate (32b) (40 mg, 55% yield) as a light yellow oil; MS (ES+): 581.20 (M+1).

### Step-3: Preparation of 2-(2-((7-(3-(aminomethyl)phenyl)benzofuran-5-yl)methoxy)-4-((diethoxyphosphorylamino)methyl)phenyl)acetic acid (32c)

Compound 32c was prepared according to procedure reported in Step-8 of scheme 1 from ethyl 2-(2-((7-(3-(aminomethyl)phenyl)benzofuran-5-yl)methoxy)-4-((diethoxyphosphorylamino)methyl)phenyl)acetate (32b) (40 mg, 0.07 mmol) in THE (4 mL), methanol (4 mL) using a solution of lithium hydroxide monohydrate (23.13 mg, 0.55 mmol) in water (1 mL) and stirring for 16 h at room temperature. This gave after workup and purification by reverse-phase column chromatography [C-18 column (50 g), eluting with 0.1% aq HCl in water and acetonitrile from 0-100%]2-(2-((7-(3-(aminomethyl)phenyl)benzofuran-5-yl)methoxy)-4-((diethoxyphosphorylamino)methyl)phenyl)acetic acid (32c) (22 mg, 58% yield) HCl salt as a light yellow solid; ¹H NMR (300 MHz, DMSO-d₆) δ 12.20 (s, 1H, D₂O exchangeable), 8.40 (s, 3H, D₂O exchangeable), 8.11 (d, J=2.2 Hz, 1H), 8.01 (d, J=1.8 Hz, 1H), 7.93 (dt, J=7.4, 1.7 Hz, 1H), 7.76 (d, J=1.6 Hz, 1H), 7.65 (d, J=1.6 Hz, 1H), 7.63-7.51 (m, 2H), 7.18-7.10 (m, 2H), 7.06 (d, J=2.2 Hz, 1H), 6.86 (dd, J=7.6, 1.5 Hz, 1H), 5.48 (dt, J=11.9, 7.2 Hz, 1H), 5.26 (s, 2H), 4.14 (s, 2H), 3.93 (dd, J=11.8, 7.2 Hz, 2H), 3.88-3.73 (m, 4H), 3.57 (s, 2H), 1.12 (t, J=7.0 Hz, 6H); MS (ES+): 553.20 (M+1); Analysis calculated for C₂₉H₃₃N₂O₇P·HCl·1.75H₂O: C, 56.13; H, 6.09; N, 4.51; Found: C, 56.11; H, 5.82; N, 4.62.

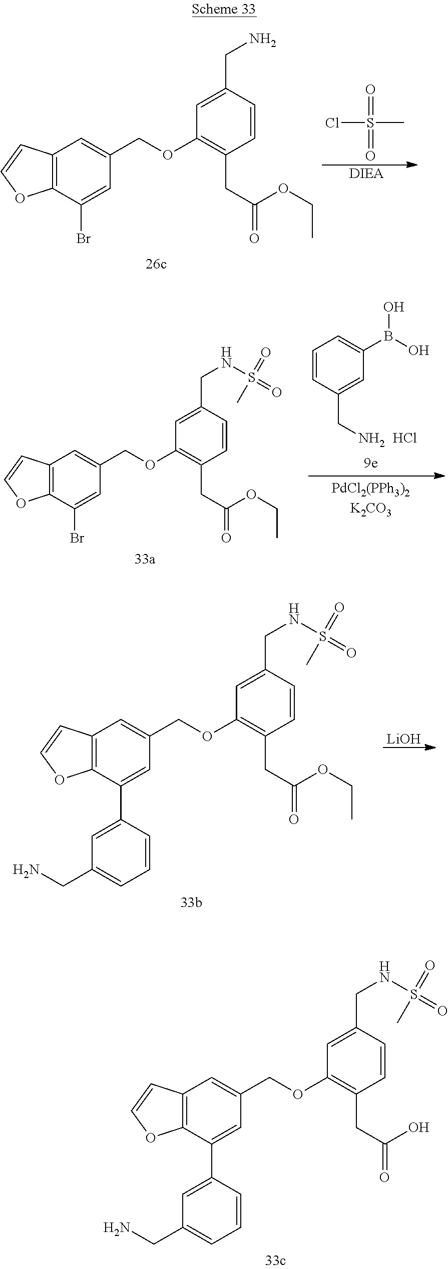

### Preparation of 2-(2-((7-(3-(aminomethyl)phenyl)benzofuran-5-yl)methoxy)-4-(methylsulfonamidomethyl)phenyl)acetic acid (33c)

### Step-1: Preparation of ethyl 2-(2-((7-bromobenzofuran-5-yl)methoxy)-4-(methylsulfonamidomethyl)phenyl)acetate (33a)

Compound 33a was prepared according to the procedure reported in step-6 of scheme 25 from ethyl 2-(4-(aminomethyl)-2-((7-bromobenzofuran-5-yl)methoxy)phenyl)acetate (26c) (300 mg, 0.72 mmol) in DCM (4 mL) using N-ethyl-N-isopropylpropan-2-amine (0.25 mL, 1.43 mmol), methanesulfonyl chloride (0.11 mL, 1.43 mmol) and stirring for 1 h at RT. This gave after workup and purification by flash column chromatography [silica gel column (24 g), eluting with DMA80 in DCM from 0-50%]ethyl 2-(2-((7-bromobenzofuran-5-yl)methoxy)-4-(methylsulfonamidomethyl)phenyl)acetate (33a) (0.26 g, 73% yield) as a clear oil; MS (ES−): 494.40 (M−1).

### Step-2: Preparation of ethyl 2-(2-((7-(3-(aminomethyl)phenyl)benzofuran-5-yl)methoxy)-4-(methylsulfonamidomethyl)phenyl)acetate (33b)

Compound 33b was prepared according to procedure reported in Step-4 of scheme 1 from ethyl 2-(2-((7-bromobenzofuran-5-yl)methoxy)-4-(methylsulfonamidomethyl)phenyl)acetate (33a) (0.26 g, 0.524 mmol) in dioxane (4 mL) using 3-(aminomethyl)phenylboronic acid hydrochloride (9e) (0.196 g, 1.048 mmol), bis(triphenylphosphine)Palladium(II)chloride [Pd(PPh₃)₂Cl₂](0.055 g, 0.079 mmol), a solution of K₂CO₃ (0.217 g, 1.571 mmol) in water (1 mL) and heating at 90° C. for 2 h on oil bath. This gave after workup and purification by flash column chromatography [silica gel (12 g), eluting with DMA-80 in DCM from 0-50%]ethyl 2-(2-((7-(3-(aminomethyl)phenyl)benzofuran-5-yl)methoxy)-4-(methylsulfonamidomethyl)phenyl)acetate (33b) (0.11 g, 40% yield) as a light-yellow oil. The submitted sample was further purified by reverse column chromatography [C18 (50 g), eluting with ACN in water (containing 0.1% HCl) from 0-100%] to give compound 33b (80 mg) HCl salt as a white solid; ¹H NMR (300 MHz, DMSO-d₆) δ 8.49 (s, 3H), 8.11 (d, J=2.2 Hz, 1H), 8.02 (d, J=2.2 Hz, 1H), 7.92 (dt, J=6.5, 2.2 Hz, 1H), 7.75 (d, J=1.6 Hz, 1H), 7.66-7.54 (m, 4H), 7.20 (d, J=7.6 Hz, 1H), 7.17-7.15 (m, 1H), 7.07 (d, J=2.2 Hz, 1H), 6.93-6.87 (m, 1H), 5.24 (s, 2H), 4.21-4.07 (m, 4H), 3.92 (q, J=7.1 Hz, 2H), 3.61 (s, 2H), 2.82 (s, 3H), 0.98 (t, J=7.1 Hz, 3H); MS (ES+): 523.20 (M+1).

### Step-3: Preparation of 2-(2-((7-(3-(aminomethyl)phenyl)benzofuran-5-yl)methoxy)-4-(methylsulfonamidomethyl)phenyl)acetic acid (33c)

Compound 33c was prepared according to procedure reported in Step-8 of scheme 1 from ethyl 2-(2-((7-(3-(aminomethyl)phenyl)benzofuran-5-yl)methoxy)-4-(methylsulfonamidomethyl)phenyl)acetate (33b) (0.11 g, 0.21 mmol) in THE (4 mL), methanol (4 mL) using a solution of lithium hydroxide monohydrate (0.07 g, 1.68 mmol) in water (1 mL) and stirring for 16 h at room temperature. This gave after workup 2-(2-((7-(3-(aminomethyl)phenyl)benzofuran-5-yl)methoxy)-4-(methylsulfonamidomethyl)phenyl)acetic acid (33c) (0.10 g, 94% yield) HCl salt as a white solid; ¹H NMR (300 MHz, DMSO-d₆) δ 12.23 (s, 1H, D₂O exchangeable), 8.33 (s, 3H, D₂O exchangeable), 8.11 (d, J=2.2 Hz, 1H), 8.00 (s, 1H), 7.94 (dt, J=7.5, 1.7 Hz, 1H), 7.78 (d, J=1.6 Hz, 1H), 7.65 (d, J=1.6 Hz, 1H), 7.64-7.51 (m, 3H), 7.20 (d, J=7.6 Hz, 1H), 7.13 (d, J=1.5 Hz, 1H), 7.07 (d, J=2.2 Hz, 1H), 6.90 (dd, J=7.6, 1.5 Hz, 1H), 5.26 (s, 2H), 4.19-4.08 (m, 4H), 3.58 (s, 2H), 2.82 (s, 3H); MS (ES+): 495.10 (M+1); Analysis Calculated for: C₂₆H₂₆N₂O₆S·HCl·1.25H₂O. C, 56.42; H, 5.37; Cl, 6.40; N, 5.06; Found: C, 56.25; H, 5.15; Cl, 6.48; N, 5.15.

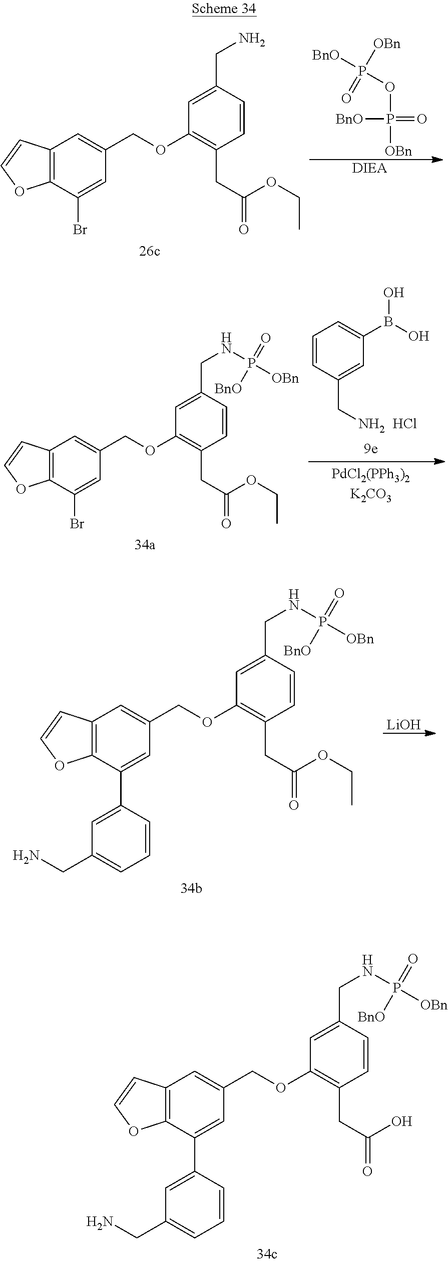

### Preparation of 2-(2-((7-(3-(aminomethyl)phenyl)benzofuran-5-yl)methoxy)-4-((bis(benzyloxy)phosphorylamino)methyl)phenyl)acetic acid (34c)

### Step-1: Preparation of ethyl 2-(4-((bis(benzyloxy)phosphorylamino)methyl)-2-((7-bromobenzofuran-5-yl)methoxy)phenyl)acetate (34a)

Compound 34a was prepared according to the procedure reported in step-6 of scheme 25 from ethyl 2-(4-(aminomethyl)-2-((7-bromobenzofuran-5-yl)methoxy)phenyl)acetate (26c) (0.4 g, 0.956 mmol) in DCM (4 mL) using N-ethyl-N-isopropylpropan-2-amine (0.25 mL, 1.43 mmol), tetrabenzyl diphosphate (0.618 g, 1.148 mmol) and stirring for 1 h at RT. This gave after workup and purification by flash column chromatography [silica gel column (24 g), eluting with EtOAc in hexane from 0-100%]ethyl 2-(4-((bis(benzyloxy)phosphorylamino)methyl)-2-((7-bromobenzofuran-5-yl)methoxy)phenyl)acetate (34a) (0.45 g, 69% yield) as a white oil; MS (ES+): 678.10 (M+1); 700.10 (M+Na).

### Step-2: Preparation of ethyl 2-(2-((7-(3-(aminomethyl)phenyl)benzofuran-5-yl)methoxy)-4-((bis(benzyloxy)phosphorylamino)methyl)phenyl)acetate (34b)

Compound 34b was prepared according to procedure reported in Step-4 of scheme 1 from ethyl 2-(4-((bis(benzyloxy)phosphorylamino)methyl)-2-((7-bromobenzofuran-5-yl)methoxy)phenyl)acetate (34a) (0.45 g, 0.66 mmol) in dioxane (4 mL) using 3-(aminomethyl)phenylboronic acid hydrochloride (9e) (0.249 g, 1.33 mmol), bis(triphenylphosphine)Palladium(II)chloride [Pd(PPh₃)₂Cl₂](0.07 g, 0.10 mmol), a solution of K₂CO₃ (0.28 g, 1.99 mmol) in water (1 mL) and heating at 90° C. for 2 h on oil bath. This gave after workup and purification by flash column chromatography [silica gel (12 g), eluting with DMA-80 in DCM from 0-50%]ethyl 2-(2-((7-(3-(aminomethyl)phenyl)benzofuran-5-yl)methoxy)-4-((bis(benzyloxy)phosphorylamino)methyl)phenyl)acetate (34b) (0.2 g, 43% yield) as a light yellow oil; MS (ES+): 705.20 (M+1).

### Step-3: Preparation of 2-(2-((7-(3-(aminomethyl)phenyl)benzofuran-5-yl)methoxy)-4-((bis(benzyloxy)phosphorylamino)methyl)phenyl)acetic acid (34c)

Compound 34c was prepared according to procedure reported in Step-8 of scheme 1 from ethyl 2-(2-((7-(3-(aminomethyl)phenyl)benzofuran-5-yl)methoxy)-4-((bis(benzyloxy)phosphorylamino)methyl)phenyl)acetate (34b) (0.48 g, 0.68 mmol) in THF (4 mL), methanol (4 mL) using a solution of lithium hydroxide monohydrate (0.11 g, 2.72 mmol) in water (1 mL) and stirring for 16 h at room temperature. This gave after workup and purification by reverse column chromatography [C18 (50 g), eluting with ACN in water (containing 0.1% HCl) from 0-100%]2-(2-((7-(3-(aminomethyl)phenyl)benzofuran-5-yl)methoxy)-4-((bis(benzyloxy)phosphorylamino)methyl)phenyl)acetic acid (34c) (0.26 g, 56% yield) HCl salt as a white solid; ¹H NMR (300 MHz, DMSO-d₆) δ 12.20 (s, 1H, D₂O exchangeable), 8.39 (s, 3H, D₂O exchangeable), 8.09 (d, J=2.2 Hz, 1H), 7.99 (d, J=2.0 Hz, 1H), 7.90 (dt, J=7.3, 1.8 Hz, 1H), 7.71 (d, J=1.6 Hz, 1H), 7.62-7.52 (m, 3H), 7.38-7.24 (m, 10H), 7.18-7.11 (m, 2H), 7.03 (d, J=2.2 Hz, 1H), 6.87 (dd, J=7.6, 1.5 Hz, 1H), 5.82 (dt, J=13.5, 7.2 Hz, 1H), 5.15 (s, 2H), 4.98-4.81 (m, 4H), 4.14 (s, 2H), 3.99 (dd, J=12.4, 7.2 Hz, 2H), 3.56 (s, 2H); MS (ES+): 677.20 (M+1); Analysis Calculated for: C₃₉H₃₇N₂O₇P·HCl·1.25H₂O. C, 63.67; H, 5.55; Cl, 4.82; N, 3.81; Found: C, 63.99; H, 5.48; Cl, 5.02; N, 3.83.

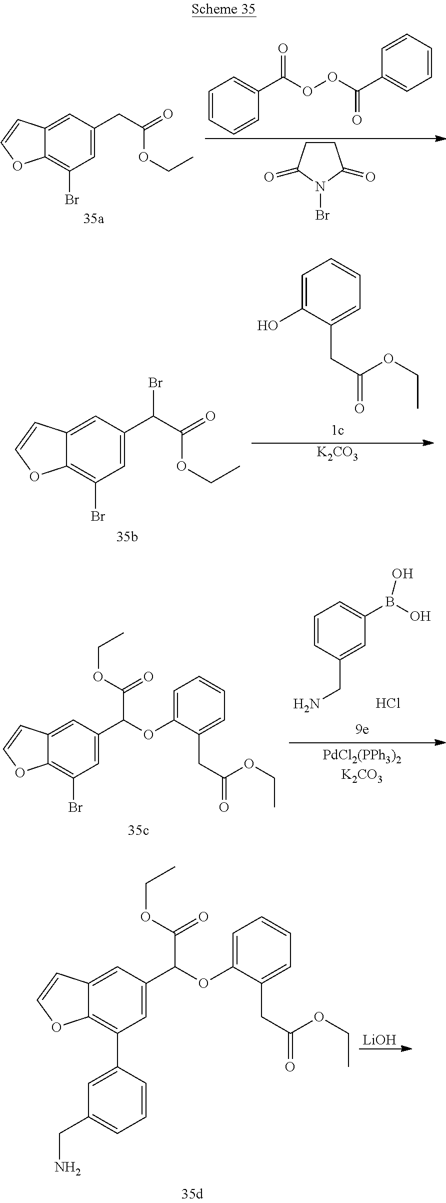

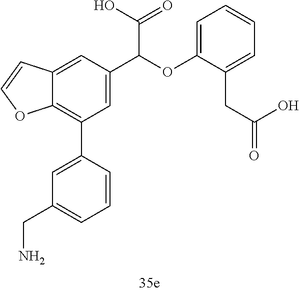 [SMILES: NCc1cccc(-c2cc(C(Oc3ccccc3CC(=O)O)C(=O)O)cc3ccoc23)c1]

### Preparation of 2-(7-(3-(aminomethyl)phenyl)benzofuran-5-yl)-2-(2-(carboxymethyl)phenoxy)acetic acid (35e)

### Step-1: Preparation of ethyl 2-bromo-2-(7-bromobenzofuran-5-yl)acetate (35b)

A solution of ethyl 2-(7-bromobenzofuran-5-yl)acetate (35a) (0.5 g, 1.77 mmol; CAS #1260774-41-1), NBS (0.35 g, 1.94 mmol) and benzoic peroxyanhydride (0.06 g, 0.27 mmol) was heated to reflux in CCl₄ (10 mL) for 4 h. The mixture was cooled to room temperature and filtered. The filtrate was concentrated in vacuo to give ethyl 2-bromo-2-(7-bromobenzofuran-5-yl)acetate (35b) (0.2 g, 31% yield) as a pale yellow oil, which was used in the next step without further purification; MS (ES+): 360.90 & 362.90 (M+1).

### Step-2: Preparation of ethyl 2-(7-bromobenzofuran-5-yl)-2-(2-(2-ethoxy-2-oxoethyl)phenoxy)acetate (35c)

Compound 35c was prepared according to the procedure reported in step-4 of scheme 14 from ethyl 2-bromo-2-(7-bromobenzofuran-5-yl)acetate (35b) (0.2 g, 0.55 mmol) in acetonitrile (5 mL) using ethyl 2-(2-hydroxyphenyl)acetate (1c) (0.11 g, 0.61 mmol), K₂CO₃ (0.23 g, 1.66 mmol) and stirring at room temperature for 15 h. This gave after workup and purification by flash column chromatography (silica gel, (24) g, eluting with 0 to 60% EtOAc in hexane) ethyl 2-(7-bromobenzofuran-5-yl)-2-(2-(2-ethoxy-2-oxoethyl)phenoxy)acetate (35c) (0.14 g, 55% yield) as a white oil; MS (ES+): 483.90 & 485.90 (M+Na).

### Step-3: Preparation of ethyl 2-(7-(3-(aminomethyl)phenyl)benzofuran-5-yl)-2-(2-(2-ethoxy-2-oxoethyl)phenoxy)acetate (35d)

Compound 35d was prepared according to procedure reported in Step-4 of scheme 1 from ethyl 2-(7-bromobenzofuran-5-yl)-2-(2-(2-ethoxy-2-oxoethyl)phenoxy)acetate (35c) (0.14 g, 0.30 mmol) in dioxane (4 mL) using 3-(aminomethyl)phenylboronic acid hydrochloride (9e) (0.11 g, 0.61 mmol), bis(triphenylphosphine)Palladium(II)chloride [Pd(PPh₃)₂Cl₂](0.03 g, 0.05 mmol), a solution of K₂CO₃ (0.13 g, 0.91 mmol) in water (1 mL) and heating at 90° C. for 2 h on oil bath. This gave after workup and purification by flash column chromatography [silica gel (24 g), eluting with DMA-80 in DCM from 0-50%]ethyl 2-(7-(3-(aminomethyl)phenyl)benzofuran-5-yl)-2-(2-(2-ethoxy-2-oxoethyl)phenoxy)acetate (35d) (0.06 g, 41% yield) as a yellow oil; MS (ES+): 488.10 (M+1).

### Step-4: Preparation of 2-(7-(3-(aminomethyl)phenyl)benzofuran-5-yl)-2-(2-(carboxymethyl)phenoxy)acetic acid (35e)

Compound 35e was prepared according to procedure reported in Step-8 of scheme 1 from ethyl 2-(7-(3-(aminomethyl)phenyl)benzofuran-5-yl)-2-(2-(2-ethoxy-2-oxoethyl)phenoxy)acetate (35d) (50 mg, 0.10 mmol) in THE (4 mL), methanol (4 mL) using a solution of lithium hydroxide monohydrate (34.4 mg, 0.82 mmol) in water (1 mL) and stirring for 15 h at room temperature. This gave after workup and purification by reverse-phase column chromatography [C-18 column (50 g), eluting with 0.1% aq HCl in water and acetonitrile from 0-100%]2-(7-(3-(aminomethyl)phenyl)benzofuran-5-yl)-2-(2-(carboxymethyl)phenoxy)acetic acid (35e) (40 mg, 90% yield) HCl salt as a white solid; ¹H NMR (300 MHz, DMSO-d₆) δ 12.82 (s, 2H, D₂O exchangeable), 8.37 (s, 2H, D₂O exchangeable), 8.13 (d, J=2.2 Hz, 1H), 7.97 (s, 1H), 7.95-7.87 (m, 2H), 7.78 (d, J=1.7 Hz, 1H), 7.65-7.52 (m, 2H), 7.22 (m, 2H), 7.11 (d, J=2.2 Hz, 1H), 7.04-6.87 (m, 2H), 6.02 (s, 1H), 4.15 (s, 2H), 3.85-3.56 (m, 2H); MS (ES+): 432.05 (M+1); MS (ES−): 430.10 (M−1); Analysis calculated for: C₂₅H₂₁NO₆·HCl·2H₂O. C, 59.59; H, 5.20; Cl, 7.03; N, 2.78; Found: C, 59.85; H, 5.17; Cl, 7.33; N, 2.90.

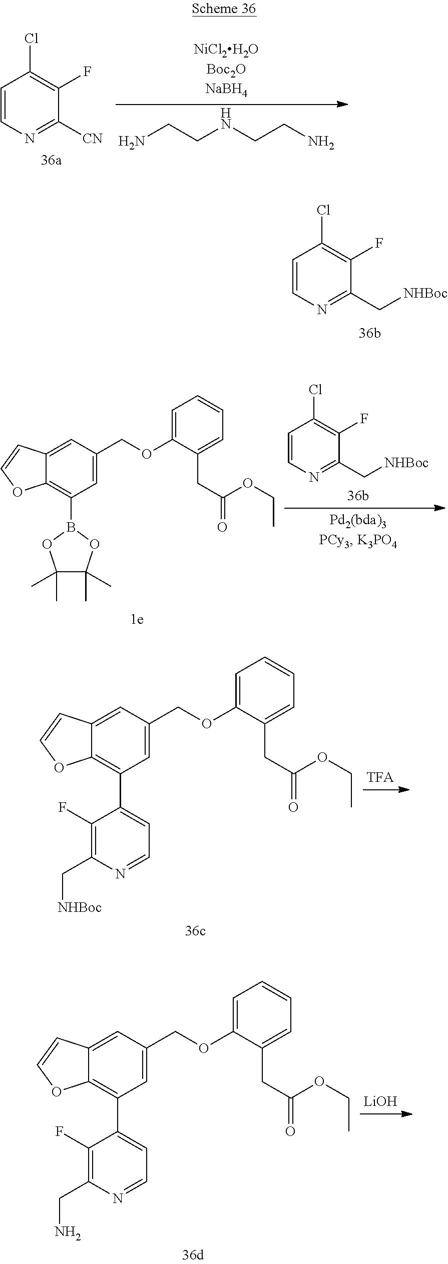

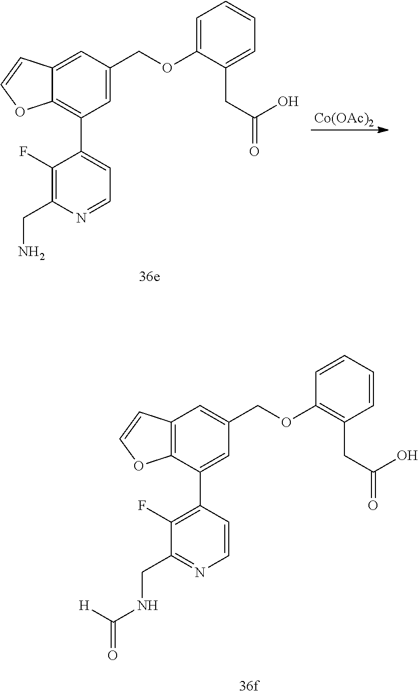 [SMILES: NCc1nccc(-c2cc(COc3ccccc3CC(=O)O)cc3ccoc23)c1F ; O=CNCc1nccc(-c2cc(COc3ccccc3CC(=O)O)cc3ccoc23)c1F ; "Co(OAc)2"]

### Preparation of 2-(2-((7-(3-fluoro-2-(formamidomethyl)pyridin-4-yl)benzofuran-5-yl)methoxy)phenyl)acetic acid (36f)

### Step-1: Preparation of tert-butyl ((4-chloro-3-fluoropyridin-2-yl)methyl)carbamate (36b)

Compound 36b was prepared according to procedure reported in Step-2 of scheme 26 4-chloro-3-fluoropicolinonitrile (36a) (0.26 g, 1.661 mmol; CAS #1155847-43-0) in methanol (15 mL), using BOC-Anhydride (0.54 g, 2.49 mmol), nickel(II) chloride hydrate (0.025 g, 0.17 mmol), sodium borohydride (0.19 g, 4.98 mmol) and N1-(2-aminoethyl)ethane-1,2-diamine (0.9 mL, 8.30 mmol) for quenching. This gave after workup and purification by flash column chromatography [silica gel 12 g, eluting with EtOAc in hexanes 0 to 60%] to afford tert-butyl ((4-chloro-3-fluoropyridin-2-yl)methyl)carbamate (36b) (0.26 g, 60% yield) as a crystalline solid.

### Step-2: Preparation of ethyl 2-(2-((7-(2-(((tert-butoxycarbonyl)amino)methyl)-3-fluoropyridin-4-yl)benzofuran-5-yl)methoxy)phenyl)acetate (36c)

Compound 36c was prepared according to the procedure reported in step-7 of scheme 25 from ethyl 2-(2-((7-(4,4,5,5-tetramethyl-1,3,2-dioxaborolan-2-yl)benzofuran-5-yl)methoxy)phenyl)acetate (1e) (243 mg, 0.56 mmol) in dioxane (6 mL) using tert-butyl ((4-chloro-3-fluoropyridin-2-yl)methyl)carbamate (36b) (145 mg, 0.56 mmol), tripotassium phosphate (3M aqueous, 0.32 mL, 0.95 mmol), tricyclohexylphosphine (47 mg, 0.17 mmol) and Pd₂(dba)₃ (51 mg, 0.056 mmol) under an Ar atmosphere and heating at 120° C. for 90 min in a microwave. This gave after workup, purification by flash column chromatography [silica (12g), eluting with EtOAc in hexane from 0-70%]ethyl 2-(2-((7-(2-(((tert-butoxycarbonyl)amino)methyl)-3-fluoropyridin-4-yl)benzofuran-5-yl)methoxy)phenyl)acetate (36c) (185 mg, 62% yield) as a white solid; ¹H NMR (300 MHz, DMSO-d₆) δ 8.50 (d, J=5.0 Hz, 1H), 8.11 (d, J=2.1 Hz, 1H), 7.85 (d, J=1.5 Hz, 1H), 7.63 (t, J=5.3 Hz, 1H), 7.53 (s, 1H), 7.34-7.28 (m, 1H), 7.28-7.19 (m, 2H), 7.15-7.08 (m, 2H), 6.91 (td, J=7.3, 1.3 Hz, 1H), 5.25 (s, 2H), 3.95-3.86 (m, 2H), 3.62 (s, 2H), 3.34 (s, 2H), 1.39 (s, 9H), 1.00-0.92 (m, 3H); MS (ES+): 535.4 (M+1); 557.4 (M+Na); (ES−): 534.3 (M−1).

### Step-3: Preparation of ethyl 2-(2-((7-(2-(aminomethyl)-3-fluoropyridin-4-yl)benzofuran-5-yl)methoxy)phenyl)acetate (36d)

Compound 36d was prepared according to the procedure reported in step-7 of scheme 23 from ethyl 2-(2-((7-(2-(((tert-butoxycarbonyl)amino)methyl)-3-fluoropyridin-4-yl)benzofuran-5-yl)methoxy)phenyl)acetate (36c) (182 mg, 0.34 mmol) in DCM (5 mL) using TFA (0.26 mL, 3.40 mmol). This gave after workup and purification by flash column chromatography [silica (12g), eluting with DMA80 in DCM from 0-50%] followed by reverse phase column purification [C18 (50 g), eluting with ACN in water (containing 0.1% HCl) from 0-100%]ethyl 2-(2-((7-(2-(aminomethyl)-3-fluoropyridin-4-yl)benzofuran-5-yl)methoxy)phenyl)acetate (36d) (136 mg, 92% yield) HCl salt as a white solid; ¹H NMR (300 MHz, DMSO-d₆) δ 8.63 (d, J=5.0 Hz, 4H, partially D₂O exchangeable), 8.12 (d, J=2.2 Hz, 1H), 7.89 (d, J=1.6 Hz, 1H), 7.80 (t, J=5.3 Hz, 1H), 7.57 (s, 1H), 7.31-7.19 (m, 2H), 7.16-7.09 (m, 2H), 6.92 (td, J=7.4, 1.1 Hz, 1H), 5.27 (s, 2H), 4.36 (s, 2H), 3.94 (q, J=7.1, 1.3 Hz, 2H), 3.63 (s, 2H), 1.00 (t, J=7.1, 1.3 Hz, 3H); ¹⁹F NMR (282 MHz, DMSO-d₆) δ −128.52; MS (ES+): 435.3 (M+1); (ES−): 469.3 (M+Cl).

### Step-4: Preparation of 2-(2-((7-(2-(aminomethyl)-3-fluoropyridin-4-yl)benzofuran-5-yl)methoxy)phenyl)acetic acid (36e)

Compound 36e was prepared according to the procedure reported in step-6 of scheme 1, from ethyl 2-(2-((7-(2-(aminomethyl)-3-fluoropyridin-4-yl)benzofuran-5-yl)methoxy)phenyl)acetate (36d) (61 mg, 0.14 mmol) in MeOH/THF (10 mL) using a solution of lithium hydroxide monohydrate (18 mg, 0.42 mmol) in water (1 mL). This gave after workup and purification by reverse phase column [C18 (50 g), eluting with ACN in water (containing 0.1% HCl) from 0-100%]2-(2-((7-(2-(aminomethyl)-3-fluoropyridin-4-yl)benzofuran-5-yl)methoxy)phenyl)acetic acid (36e) (32 mg, 56% yield) HCl salt as a white solid; ¹H NMR (300 MHz, DMSO-d₆) δ 8.73-8.58 (m, 4H, partially D₂O exchangeable), 8.11 (d, J=2.1 Hz, 1H), 7.92 (d, J=1.6 Hz, 1H), 7.80 (t, J=5.3 Hz, 1H), 7.61 (s, 1H), 7.28-7.19 (m, 2H), 7.14-7.06 (m, 2H), 6.91 (t, J=7.3 Hz, 1H), 5.29 (s, 2H), 4.39-4.33 (m, 2H), 3.59 (s, 2H); ¹⁹F NMR (282 MHz, DMSO-d₆) δ −128.34; MS (ES+): 407.2 (M+1); 429.2 (M+Na); (ES−): 405.3 (M−1); 441.3 (M+Cl). The HCl salt of compound 36e (3.0 g) was suspended in saturated solution of NaHCO₃ (60.0 mL) and stirred for 30 minutes. Suspension was filtered and washed by water and dried to afford 2.2 g, no chloride content was determined based on elemental analysis. The sample (1 g) was dissolved in methanol by adding small amount of aqueous ammonium hydroxide and purified by flash column chromatography [silica gel 100 g, eluting with DMA-80 in DCM from 0-100%] to furnish 2-(2-((7-(2-(aminomethyl)-3-fluoropyridin-4-yl)benzofuran-5-yl)methoxy)phenyl)acetic acid (36e) (0.82 g, 82%) free base as a white solid. ¹H NMR (300 MHz, DMSO-d₆) δ 8.51 (d, J=5.0 Hz, 1H), 8.09 (d, J=2.2 Hz, 1H), 7.87 (d, J=1.6 Hz, 1H), 7.69-7.55 (m, 2H), 7.28-7.14 (m, 2H), 7.12-7.01 (m, 2H), 6.89 (t, J=7.3 Hz, 1H), 5.27 (s, 2H), 4.00-3.95 (m, 2H), 3.55 (s, 2H); MS (ES+): 407.3 (M+1).

### Step-5: Preparation of 2-(2-((7-(3-fluoro-2-(formamidomethyl)pyridin-4-yl)benzofuran-5-yl)methoxy)phenyl)acetic acid (36f)

To a stirred solution of 2-(2-((7-(2-(aminomethyl)-3-fluoropyridin-4-yl)benzofuran-5-yl)methoxy)phenyl)acetic acid (36e) (free base 0.200 g, 492 μmol) in a sealed tube was added DMF (2.5 mL) followed by cobaltous acetate (24.5 mg, 98.4 μmol) under a positive flow of nitrogen and heated at 150° C. for 3 h. The reaction mixture was cooled to room temperature, was diluted with a minimum amount of DMSO and filtered. The residue obtained was purified by reverse-phase column chromatography [EZ-PREP, C-18 column, 50 g, eluting with acetonitrile in water (containing 0.1% HCl) from 0-100%] to afford 2-(2-((7-(3-fluoro-2-(formamidomethyl)pyridin-4-yl)benzofuran-5-yl)methoxy)phenyl)acetic acid (36f) (0.0150 g, 7.02%) as an off-white solid; ¹H NMR (300 MHz, DMSO-d₆) δ 8.63 (d, J=5.0 Hz, 1H), 8.53 (t, J=5.3 Hz, 1H), 8.17-8.08 (m, 2H), 7.90 (dd, J=7.6, 1.6 Hz, 1H), 7.74 (dt, J=40.5, 5.2 Hz, 1H), 7.59 (d, J=7.2 Hz, 1H), 7.23 (ddd, J=9.1, 7.5, 1.6 Hz, 2H), 7.11 (d, J=1.9 Hz, 1H), 7.08 (d, J=2.0 Hz, 1H), 6.90 (td, J=7.4, 1.2 Hz, 1H), 5.28 (d, J=2.7 Hz, 2H), 4.59 (dd, J=5.9, 2.0 Hz, 2H), 3.58 (s, 2H); ¹⁹F NMR (282 MHz, DMSO-d₆) δ −128.85, −128.87; MS (ES+): 435.3 (M+1), (ES−) 433.3 (M−1); Analysis calculated for C₂₄H₁₉FN₂O₅·0.5H₂O: C, 65.01; H, 4.55; N, 6.32; Found: C, 64.80; H, 4.75; N, 6.27.

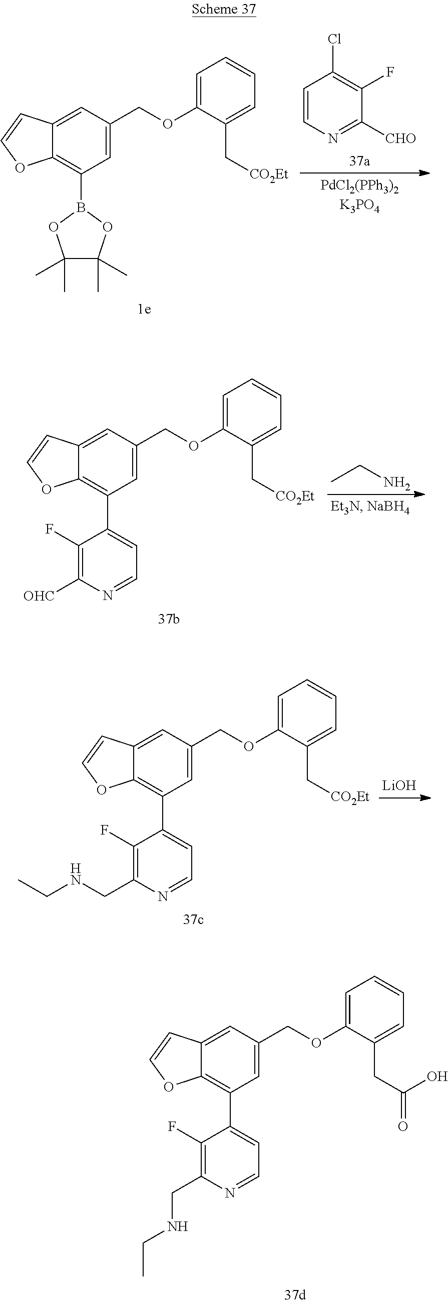

### Preparation of 2-(2-((7-(2-((ethylamino)methyl)-3-fluoropyridin-4-yl)benzofuran-5-yl)methoxy)phenyl)acetic acid (37d)

### Step-1: Preparation of ethyl 2-(2-((7-(3-fluoro-2-formylpyridin-4-yl)benzofuran-5-yl)methoxy)phenyl)acetate (37b)

Compound 37b was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (23.0 g, 52.71 mmol) in DME (345 mL) using 4-chloro-3-fluoropicolinaldehyde (37a) (CAS #1260878-78-1; 10.34 g, 64.82 mmol), bis(triphenylphosphine)Palladium(II)chloride [Pd(PPh₃)₂Cl₂](5.5 g, 7.83 mmol), a solution of K₃PO₄ (24.17 g, 113.86 mmol) in water (46 mL) and heating at 70° C. for 2 h on oil bath. This gave after workup and purification by flash column chromatography [silica gel, eluting with 30% EtOAc in n-heptane]ethyl 2-(2-((7-(3-fluoro-2-formylpyridin-4-yl)benzofuran-5-yl)methoxy)phenyl)acetate (37b) (8.5 g, 37% yield) as a yellow solid.

### Step-2: Preparation of ethyl 2-(2-((7-(2-((ethylamino)methyl)-3-fluoropyridin-4-yl)benzofuran-5-yl)methoxy)phenyl)acetate (37c)

To a stirred solution of ethyl 2-(2-((7-(3-fluoro-2-formylpyridin-4-yl)benzofuran-5-yl)methoxy)phenyl)acetate (37b) (8.0 g, 18.45 mmol) in THF (160 mL) was added ethyl amine (0.83 g, 18.45 mmol), triethyl amine (0.56 g, 5.53 mmol) at RT and stirred for 1 h. To this reaction mixture was added portion wise NaBH₄ (1.04 g, 27.68 mmol) at room temperature and stirred for 30 min. The reaction mixture was quenched with water (80 mL) and extracted with EtOAc (2×500 mL). The combined organics were washed with brine, dried, filtered and concentrated in vacuum. The obtained residue was purified by flash column chromatography [silica gel, eluting with 50% EtOAc in n-heptane] to afford ethyl 2-(2-((7-(2-((ethylamino)methyl)-3-fluoropyridin-4-yl)benzofuran-5-yl)methoxy)phenyl)acetate (37c) (8.0 g, 94% yield) as a yellow liquid.

### Step-3: 2-(2-((7-(2-((ethylamino)methyl)-3-fluoropyridin-4-yl)benzofuran-5-yl)methoxy)phenyl)acetic acid (37d)

Compound 37d was prepared according to procedure reported in Step-8 of scheme 1 from ethyl 2-(2-((7-(2-((ethylamino)methyl)-3-fluoropyridin-4-yl)benzofuran-5-yl)methoxy)phenyl)acetate (37c) (4.5 g, 9.7 mmol) in THE (45 mL), methanol (45 mL) and using a solution of lithium hydroxide monohydrate (1.22 g, 29.18 mmol) in water (45 mL) and heating for 1 h at 40° C. This gave after workup and purification by flash column chromatography [silica gel, eluting with 10% MeOH in DCM]2-(2-((7-(2-((ethylamino)methyl)-3-fluoropyridin-4-yl)benzofuran-5-yl)methoxy)phenyl)acetic acid (37d) (0.96 g, 23% yield) as a yellow solid; ¹H NMR (300 MHz, DMSO-d₆) δ 8.53 (d, J=4.9 Hz, 1H), 8.09 (d, J=2.2 Hz, 1H), 7.88 (d, J=1.7 Hz, 1H), 7.67 (t, J=5.3 Hz, 1H), 7.61 (s, 1H), 7.27-7.17 (m, 2H), 7.12-7.05 (m, 2H), 6.90 (t, J=7.3 Hz, 1H), 5.28 (s, 2H), 4.05 (d, J=2.3 Hz, 2H), 3.57 (s, 2H), 2.73 (q, J=7.2 Hz, 2H), 1.10 (t, J=7.1 Hz, 3H); ¹⁹F NMR (282 MHz, DMSO-d₆) δ −128.85; MS (ES+): 435.3 (M+1); (ES−) 433.3 (M−1); Analysis calculated for: C₂₅H₂₃FN₂O₄·1.5H₂O: C, 65.07; H, 5.68; N, 6.07; Found: C, 65.34; H, 5.75; N, 5.76.

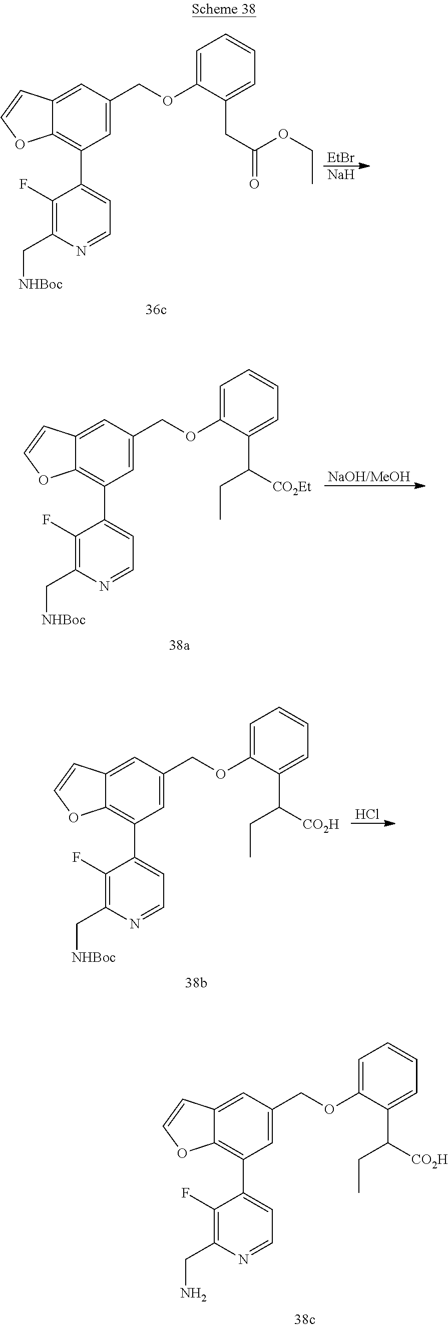

### Preparation of 2-(2-((7-(2-(aminomethyl)-3-fluoropyridin-4-yl)benzofuran-5-yl)methoxy)phenyl)butanoic acid (38c)

### Step-1: Preparation of ethyl 2-(2-((7-(2-(((tert-butoxycarbonyl)amino)methyl)-3-fluoropyridin-4-yl)benzofuran-5-yl)methoxy)phenyl)butanoate (38a)

To a stirred solution of DMF (80.0 mL) and NaH (2.23 gm, 93.18 mmol) was added a solution of ethyl 2-(2-((7-(2-(((tert-butoxycarbonyl)amino)methyl)-3-fluoropyridin-4-yl)benzofuran-5-yl)methoxy)phenyl)acetate (36c) (20.0 g, 37.41 mmol) in DMF (80.0 mL) under a nitrogen atmosphere at 0° C. The reaction mixture was stirred at 0° C. for 1 h, added bromoethane (3.26 gm, 29.93 mmol) dropwise at 0° C. and stirred at 0° C. for 1 h under nitrogen. The reaction mixture was poured into water (2.0 L) with stirring and the precipitate obtained was collected by filtration. The obtained residue was dissolved in EtOAc (200 mL) dried, filtered and concentrated in vacuum to afford ethyl 2-(2-((7-(2-(((tert-butoxycarbonyl)amino)methyl)-3-fluoropyridin-4-yl)benzofuran-5-yl)methoxy)phenyl)butanoate (38a) (15.0 g, 71% yield) as a yellow sticky semi-solid; MS (ES+): 563.2 (M+1).

### Step-2: Preparation of 2-(2-((7-(2-(((tert-butoxycarbonyl)amino)methyl)-3-fluoropyridin-4-yl)benzofuran-5-yl)methoxy)phenyl)butanoic acid (38b)

To a stirred solution of ethyl 2-(2-((7-(2-(((tert-butoxycarbonyl)amino)methyl)-3-fluoropyridin-4-yl)benzofuran-5-yl)methoxy)phenyl)butanoate (38a) (12.0 g, 21.32 mmol) in THE (120 mL) was added MeOH (96 mL) and a solution of NaOH (2.55 gm, 63.98 mmol) in water (96 mL) at room temperature and heated at 55° C. for 3 h. The reaction mixture was cooled, concentrated in vacuum and diluted with 100 mL of water. The reaction mixture was cooled to 0° C. and acidified to pH 2-3 using a solution of 10% KHSO₄. The reaction mixture was stirred for 1 h at 0° C. and the solid obtained was collected by filtration to afford 2-(2-((7-(2-(((tert-butoxycarbonyl)amino)methyl)-3-fluoropyridin-4-yl)benzofuran-5-yl)methoxy)phenyl)butanoic acid (38b) (9.1 gm, 80% yield) as a yellow solid; MS (ES+): 535.2 (M+1).

### Step-3: 2-(2-((7-(2-(aminomethyl)-3-fluoropyridin-4-yl)benzofuran-5-yl)methoxy)phenyl)butanoic acid (38c)

To a stirred solution of 2-(2-((7-(2-(((tert-butoxycarbonyl)amino)methyl)-3-fluoropyridin-4-yl)benzofuran-5-yl)methoxy)phenyl)butanoic acid (38b) (6.0 g, 11.22 mmol) in EtOAc (72 mL) was added 15% MTBE·HCl (60 mL) at RT and heated at 40° C. for 1 h. The solid formed was isolated by decanting the excess solvent and triturating with EtOAc (30 mL) to afford 2-(2-((7-(2-(aminomethyl)-3-fluoropyridin-4-yl)benzofuran-5-yl)methoxy)phenyl)butanoic acid (38c) (3.8 g, 68% yield) HCl salt as a yellow solid; ¹H NMR (300 MHz, DMSO-d₆) δ 8.64 (t, J=5.4 Hz, 4H), 8.20-8.05 (m, 1H), 7.94 (s, 1H), 7.81 (t, J=5.3 Hz, 1H), 7.63 (s, 1H), 7.30-7.19 (m, 2H), 7.18-7.06 (m, 2H), 6.94 (t, J=7.4 Hz, 1H), 5.30 (s, 2H), 4.36 (q, J=6.0 Hz, 2H), 3.84 (t, J=7.4 Hz, 1H), 1.95 (dp, J=14.6, 7.2 Hz, 1H), 1.67 (dt, J=13.9, 7.3 Hz, 1H), 0.80 (t, J=7.3 Hz, 3H); MS (ES+): 435.5 (M+1); Analysis calculated for C₂₅H₂₃FN₂O₄·1.35HCl·H₂O: C, 59.85; H, 5.29; Cl, 9.54; N, 5.58; Found: C, 59.59; H, 5.33; Cl, 9.87; N, 5.57.

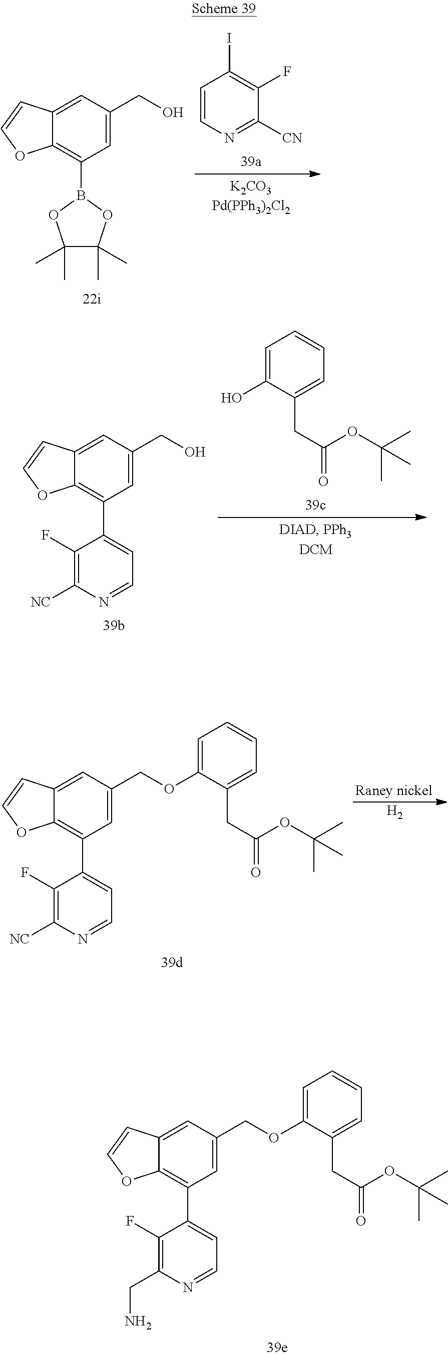

### Preparation of tert-butyl 2-(2-((7-(2-(aminomethyl)-3-fluoropyridin-4-yl)benzofuran-5-yl)methoxy)phenyl)acetate (39e)

### Step-1: Preparation of 3-fluoro-4-(5-(hydroxymethyl)benzofuran-7-yl)picolinonitrile (39b)

Compound 39b was prepared according to the procedure reported in step-4 of scheme 1 from (7-(4,4,5,5-tetramethyl-1,3,2-dioxaborolan-2-yl)benzofuran-5-yl)methanol (22i) in dioxane using 3-fluoro-4-iodopicolinonitrile (39a) (CAS #669066-35-7), PdCl₂(PPh₃)₂ and a solution of potassium carbonate in water. This gave after workup and purification 3-fluoro-4-(5-(hydroxymethyl)benzofuran-7-yl)picolinonitrile (39b); ¹HNMR (300 MHz, DMSO) δ ¹H NMR (300 MHz, DMSO-d₆) δ 8.74 (d, J=4.9 Hz, 1H), 8.15 (ddd, J=6.1, 4.8, 1.1 Hz, 1H), 8.11-8.06 (m, 1H), 7.80 (s, 1H), 7.52 (s, 1H), 7.11-7.06 (m, 1H), 5.36 (td, J=5.7, 1.1 Hz, 1H), 4.66 (d, J=5.7 Hz, 2H); MS (ES+): 269.10 (M+1).

### Step-2: Preparation of tert-butyl 2-(2-((7-(2-cyano-3-fluoropyridin-4-yl)benzofuran-5-yl)methoxy)phenyl)acetate (39d)

Compound 39d was prepared according to procedure reported in Step-2 of scheme 1 from 3-fluoro-4-(5-(hydroxymethyl)benzofuran-7-yl)picolinonitrile (39b) (5.0 g, 18.64 mmol) in THE (75 mL) using triphenylphosphine (7.8 g, 29.73 mmol), tert-butyl 2-(2-hydroxyphenyl)acetate (39c) (CAS #: 258331-10-1; 4.0 g, 19.23 mmol) and a solution of DIAD (6.0 g, 29.67 mmol) in THE (10 mL) and stirring at room temperature for 30 min. This gave after workup tert-butyl 2-(2-((7-(2-cyano-3-fluoropyridin-4-yl)benzofuran-5-yl)methoxy)phenyl)acetate (39d) (5.6 g, 66% yield) as an off white solid.

### Step-3: Preparation of tert-butyl 2-(2-((7-(2-(aminomethyl)-3-fluoropyridin-4-yl)benzofuran-5-yl)methoxy)phenyl)acetate (39e)

To a stirred solution of tert-butyl 2-(2-((7-(2-cyano-3-fluoropyridin-4-yl)benzofuran-5-yl)methoxy)phenyl)acetate (39d) (5.5 g, 11.32 mmol) in THE (75.0 mL) was added TEA (8.6 g, 7.5 mmol) and wet Raney nickel (16.5 g) at room temperature and the reaction mixture was stirred at room temperature overnight under hydrogen (1.5 bar of pressure). The reaction mixture was filtered through a pad of Celite and washed with water (50 mL) and THE (50 mL). Filtrate was extracted with EtOAc (2×55 mL) and combined organics were washed with brine (100 mL), dried, filtered and concentrated in vacuum. The residue obtained was purified by flash column chromatography [silica gel (80 g), eluting with DCM in DMA-80 from 0-50%] to afford tert-butyl 2-(2-((7-(2-(aminomethyl)-3-fluoropyridin-4-yl)benzofuran-5-yl)methoxy)phenyl)acetate (39e) (0.750 g, 17%) as a yellow syrup. (Note: Column was saturated with 8 mL of aq. ammonium hydroxide prior to running the sample); ¹H NMR (300 MHz, DMSO-d₆) δ 8.57 (d, J=5.0 Hz, 1H), 8.52 (s, 3H), 8.06 (d, J=2.2 Hz, 1H), 7.85 (d, J=1.6 Hz, 1H), 7.73 (t, J=5.3 Hz, 1H), 7.52 (s, 1H), 7.18 (td, J=7.7, 1.7 Hz, 1H), 7.13 (dd, J=7.4, 1.7 Hz, 1H), 7.07-7.00 (m, 2H), 6.84 (td, J=7.4, 1.0 Hz, 1H), 5.20 (s, 2H), 4.30 (d, J=5.4 Hz, 2H), 3.47 (s, 2H), 1.16 (s, 9H); MS (ES+): 463.10 (M+1).

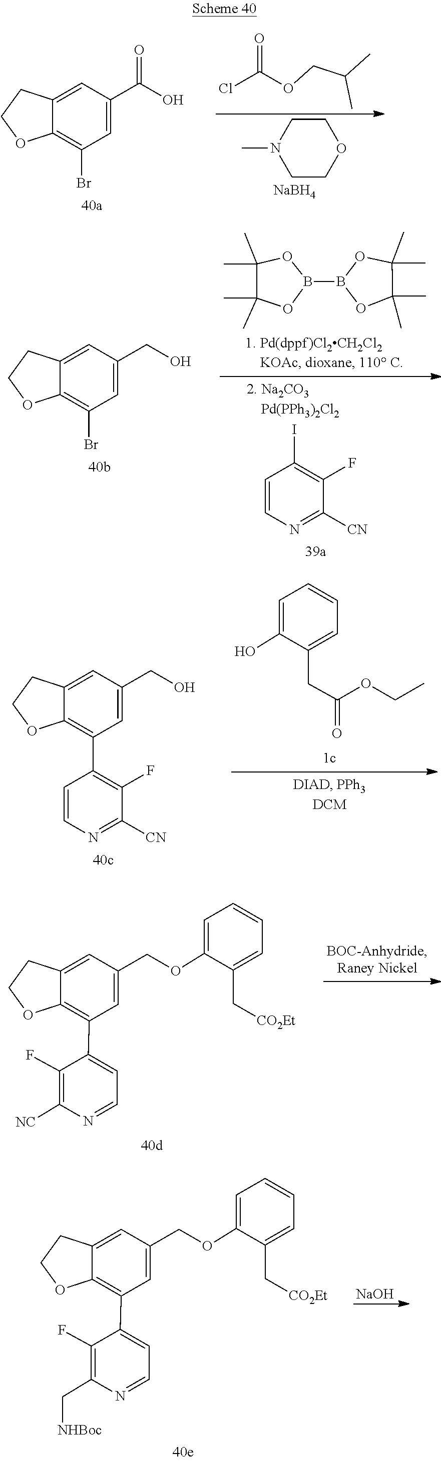

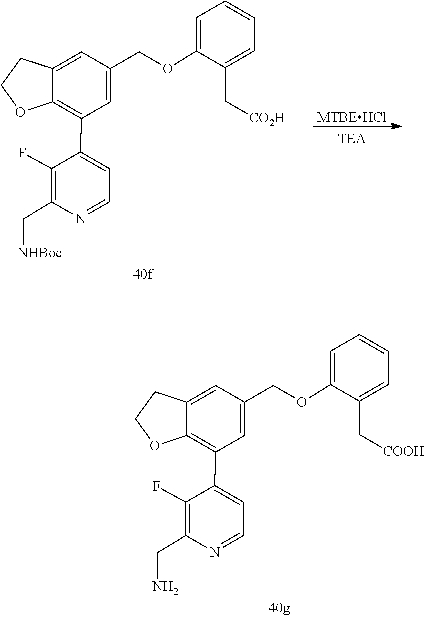 [SMILES: CC(C)(C)OC(=O)NCc1nccc(-c2cc(COc3ccccc3CC(=O)O)cc3c2OCC3)c1F ; "MTBE·HCl" ; NCc1nccc(-c2cc(COc3ccccc3CC(=O)O)cc3c2OCC3)c1F]

### Preparation of 2-(2-((7-(2-(aminomethyl)-3-fluoropyridin-4-yl)-2,3-dihydrobenzofuran-5-yl)methoxy)phenyl)acetic acid (40g)

### Step-1: Preparation of (7-bromo-2,3-dihydrobenzofuran-5-yl)methanol (40b)

Compound 40b was prepared according to the procedure reported in step-1 of scheme 1 from 7-bromo-2,3-dihydrobenzofuran-5-carboxylic acid (40a) (500 g, 2057 mmol; CAS #335671-77-7) and N-methylmorpholine (500 g, 2057 mmol) in THE (10 L) using isobutyl chloroformate (337.12 g, 2468.4 mmol), an aqueous solution of Na₂CO₃ (981.0 g, 9256.46 mmol), in water (6.6 L), NaBH₄ (326.8 g, 8639.7 mmol). This gave after workup (7-bromo-2,3-dihydrobenzofuran-5-yl)methanol (40b) (490 g) as a light pink solid; ¹H NMR (300 MHz, DMSO-d₆) δ 7.22-7.20 (m, 1H), 7.16-7.13 (m, 1H), 5.15 (td, J=5.8, 0.7 Hz, 1H), 4.59 (t, J=8.8 Hz, 2H), 4.38 (d, J=5.7 Hz, 2H), 3.27 (t, J=8.8 Hz, 2H).

### Step-2: Preparation of 3-fluoro-4-(5-(hydroxymethyl)-2,3-dihydrobenzofuran-7-yl)picolinonitrile (40c)

To a stirred solution of (7-bromo-2,3-dihydrobenzofuran-5-yl)methanol (40b) (480 g, 2095.52 mmol) in toluene (3.36 L) were added BISPIN (643.8 g, 2535.5 mmol), TPP (3.07 g, 11.73 mmol), PdCl₂(PPh₃)₂ (14.7 g, 20.95 mmol), and potassium acetate (343.4 g, 3499.51 mmol) at RT under nitrogen and heated at 110° C. for 4.0 h. The reaction mixture was cooled to RT and were added 3-fluoro-4-iodopicolinonitrile (39a) (478.1 g, 1927.87 mmol), PdCl₂(PPh₃)₂ (23.53 g, 33.52 mmol) and a solution of Na₂CO₃ (1108 g, 10456.6 mmol) in water (3.6 L) at RT under nitrogen and stirred for 14 h at 100° C. This gave after workup 3-fluoro-4-(5-(hydroxymethyl)-2,3-dihydrobenzofuran-7-yl)picolinonitrile (40c) (135.0 g, 24% yield) as an off-white solid. ¹H NMR (300 MHz, DMSO-d₆) δ 8.64 (dd, J=4.9, 0.9 Hz, 1H), 7.96 (dd, J=6.3, 4.9 Hz, 1H), 7.39-7.33 (m, 1H), 7.24 (s, 1H), 5.19 (t, J=5.6 Hz, 1H), 4.61 (t, J=8.7 Hz, 2H), 4.46 (d, J=5.6 Hz, 2H), 3.27 (t, J=8.7 Hz, 2H); MS (ES+): 271.3 (M+1).

### Step-3: Preparation of ethyl 2-(2-((7-(2-cyano-3-fluoropyridin-4-yl)-2,3-dihydrobenzofuran-5-yl)methoxy)phenyl)acetate (40d)

Compound 40d was prepared according to the procedure reported in step-2 of scheme 1 from 3-fluoro-4-(5-(hydroxymethyl)-2,3-dihydrobenzofuran-7-yl)picolinonitrile (40c) (125.0 g, 462.53 mmol) in THE (1.87 L) using triphenylphosphine (194.10 g, 740.02 mmol), ethyl 2-(2-hydroxyphenyl)acetate (1c) (116.79 g, 648.11 mmol), a solution of DIAD (150.15 g, 740.01 mmol) in THF (0.25 L). This gave after workup ethyl 2-(2-((7-(2-cyano-3-fluoropyridin-4-yl)-2,3-dihydrobenzofuran-5-yl)methoxy)phenyl)acetate (40d) (131.0 g, 66% yield) as an off white solid; ¹H NMR (300 MHz, DMSO-d₆) δ 8.88 (s, 1H), 8.67 (d, J=4.9 Hz, 1H), 7.99 (dd, J=6.2, 4.9 Hz, 1H), 7.48-7.43 (m, 1H), 7.38 (s, 1H), 7.28-7.16 (m, 1H), 7.08 (d, J=8.1 Hz, 1H), 6.98-6.85 (m, 1H), 5.06 (s, 2H), 4.64 (t, J=8.7 Hz, 2H), 3.97 (q, J=7.1 Hz, 2H), 3.61 (s, 2H), 3.32-3.21 (m, 2H), 1.05 (t, J=7.1 Hz, 3H).

### Step-4: Preparation of ethyl 2-(2-((7-(2-(((tert-butoxycarbonyl)amino)methyl)-3-fluoropyridin-4-yl)-2,3-dihydrobenzofuran-5-yl)methoxy)phenyl)acetate (40e)

To a solution of ethyl 2-(2-((7-(2-cyano-3-fluoropyridin-4-yl)-2,3-dihydrobenzofuran-5-yl)methoxy)phenyl)acetate (40d) (130.0 g, 300.61 mmol) in THE (2.6 L) at RT was added Boc anhydride (180.0 g, 4824.74 mmol), TEA (190.0 g, 1877.6 mmol) and Raney Nickel (200.0 g). The reaction mixture was hydrogenated at 38° C. at 7 Kg/cm² hydrogen pressure. This gave after workup ethyl 2-(2-((7-(2-(((tert-butoxycarbonyl)amino)methyl)-3-fluoropyridin-4-yl)-2,3-dihydrobenzofuran-5-yl)methoxy)phenyl)acetate (40e).

### Step-5: Preparation of 2-(2-((7-(2-(((tert-butoxycarbonyl)amino)methyl)-3-fluoropyridin-4-yl)-2,3-dihydrobenzofuran-5-yl)methoxy)phenyl)acetic acid (40f)

To a stirred solution of ethyl 2-(2-((7-(2-(((tert-butoxycarbonyl)amino)methyl)-3-fluoropyridin-4-yl)-2,3-dihydrobenzofuran-5-yl)methoxy)phenyl)acetate (40e) (160.0 g, 298.18 mmol) in THE (2.4 L) was added a solution of NaOH (80.0 g, 2000 mmol) in water (0.800 L), MeOH (0.800 L) and stirred at 40° C. for 2 h. The reaction mixture was concentrated to remove volatile solvent cooled to 0° C. and acidified with KHSO₄ solution (480.0 g in 1.2 L). The obtained solid was collected by filtration to afford 2-(2-((7-(2-(((tert-butoxycarbonyl)amino)methyl)-3-fluoropyridin-4-yl)-2,3-dihydrobenzofuran-5-yl)methoxy)phenyl)acetic acid (40f) (75.0 g, 46% yield) as an off white solid; ¹H NMR (300 MHz, DMSO-d₆) δ 12.20 (s, 1H), 8.44 (d, J=5.0 Hz, 1H), 7.53-7.44 (m, 2H), 7.36 (s, 1H), 7.29-7.21 (m, 3H), 7.11 (d, J=8.1 Hz, 1H), 6.94 (t, J=7.3 Hz, 1H), 5.11 (s, 2H), 4.64 (t, J=8.7 Hz, 2H), 4.41 (s, 2H), 3.59 (s, 2H), 3.38-3.23 (m, 2H), 1.44 (s, 9H).

### Step-6: Preparation of 2-(2-((7-(2-(aminomethyl)-3-fluoropyridin-4-yl)-2,3-dihydrobenzofuran-5-yl)methoxy)phenyl)acetic acid (40g)

To a stirred solution of 2-(2-((7-(2-(((tert-butoxycarbonyl)amino)methyl)-3-fluoropyridin-4-yl)-2,3-dihydrobenzofuran-5-yl)methoxy)phenyl)acetic acid (40f) (75.0 g, 147.48 mmol) in MTBE (1.5 L) was added 15% MTBE·HCl (375.0 mL) and heated at 40° C. for 5 h. TEA (80.0 mL) was added to reaction mixture at room temperature and stirred for 30 minutes. Solid obtained was collected by filtration to afford 2-(2-((7-(2-(aminomethyl)-3-fluoropyridin-4-yl)-2,3-dihydrobenzofuran-5-yl)methoxy)phenyl)acetic acid (40g) (37.0 g) triethylamine hydrochloride salt as an off white solid; ¹H NMR (300 MHz, DMSO-d₆) δ 8.46 (d, J=5.0 Hz, 1H), 7.54 (t, J=5.3 Hz, 1H), 7.43 (d, J=1.7 Hz, 1H), 7.34 (s, 1H), 7.26-7.15 (m, 2H), 7.04 (d, J=8.1 Hz, 1H), 6.88 (t, J=7.3 Hz, 1H), 5.07 (s, 2H), 4.59 (t, J=8.7 Hz, 2H), 4.15 (s, 2H), 3.52 (s, 2H), 3.27 (t, J=8.7 Hz, 2H), 2.77 (q, J=7.2 Hz, 5H), 1.07 (t, J=7.2 Hz, 8H); MS (ES+): 409.10, (ES−): 407.3 (M−1); Analysis calculated for C₂₃H₂₁FN₂O₄·1.25H₂O·HCl·N(CH₂CH₃)₃: C, 61.26; H, 7.00; Cl, 6.24; N, 7.39; Found: C, 61.07; H, 6.76; Cl, 6.55; N, 7.39.

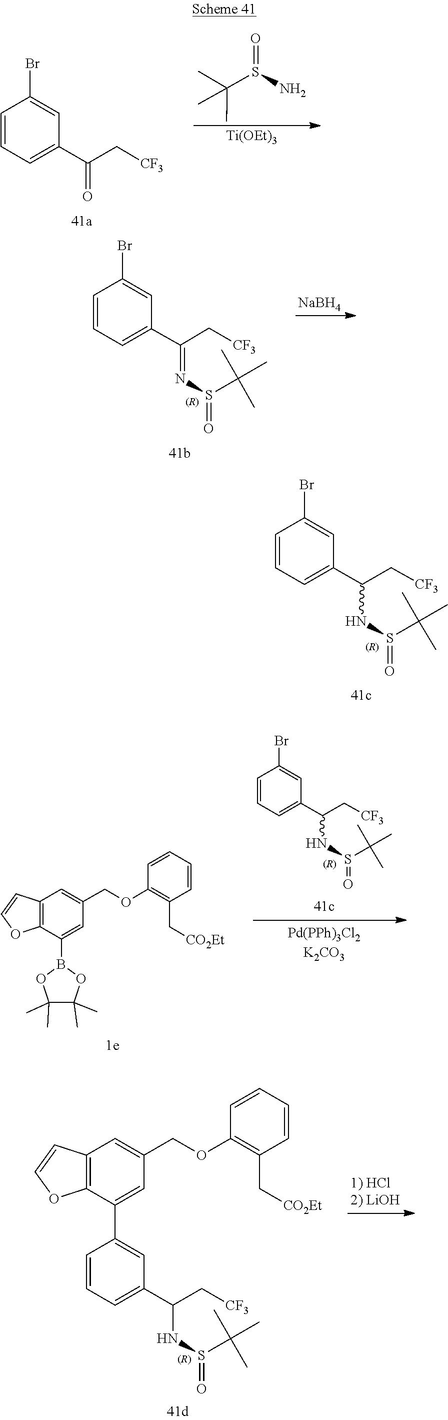

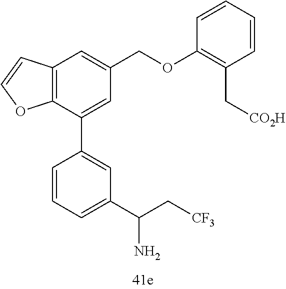 [SMILES: NC(CC(F)(F)F)c1cccc(-c2cc(COc3ccccc3CC(=O)O)cc3ccoc23)c1]

### Preparation of 2-(2-((7-(3-(1-amino-3,3,3-trifluoropropyl)phenyl)benzofuran-5-yl)methoxy)phenyl)acetic acid (41e)

### Step-1: Preparation of N-(1-(3-bromophenyl)-3,3,3-trifluoropropylidene)-2-methylpropane-2-sulfinamide (41b)

Compound 41b was prepared according to the procedure reported in step-5 of scheme 1 from 1-(3-bromophenyl)-3,3,3-trifluoropropan-1-one (41a) (0.77 g, 2.88 mmol; CAS #13541-14-5) and (R)-2-methylpropane-2-sulfinamide (0.699 g, 5.77 mmol) in tetrahydrofuran (15 mL) using tetraethoxytitanium (1.973 g, 8.65 mmol). This gave after workup and purification by flash column chromatography (silica gel, 0-30% EtOAc in hexane) N-(1-(3-bromophenyl)-3,3,3-trifluoropropylidene)-2-methylpropane-2-sulfinamide (41b) (0.33 g, 30.9% yield) as a thick yellow oil. ¹H NMR (300 MHz, DMSO-d₆) δ 8.08 (s, 1H), 7.99-7.85 (m, 1H), 7.80 (d, J=7.4 Hz, 1H), 7.49 (t, J=7.9 Hz, 1H), 4.92-4.67 (m, 1H), 4.68-4.39 (m, 1H), 1.28 (s, 9H). ¹⁹F NMR (282 MHz, DMSO) δ −59.46.

### Step-2: Preparation of (R)—N-(1-(3-bromophenyl)-3,3,3-trifluoropropyl)-2-methylpropane-2-sulfinamide (41c)

Compound 41c was prepared according to the procedure reported in step-3 of scheme 5 from N-(1-(3-bromophenyl)-3,3,3-trifluoropropylidene)-2-methylpropane-2-sulfinamide (41b) (330 mg, 0.891 mmol) in tetrahydrofuran (10 mL) using sodium borohydride (169 mg, 4.46 mmol). This gave after workup and purification by flash column chromatography [(silica gel, eluting with ethyl acetate in hexanes (0 to 30%)](R)—N-(1-(3-bromophenyl)-3,3,3-trifluoropropyl)-2-methylpropane-2-sulfinamide (41c) (260 mg, 0.698 mmol, 78% yield) as a clear colorless oil. ¹H NMR (300 MHz, DMSO-d₆) δ 7.76 (d, J=1.8 Hz, 1H), 7.54-7.45 (m, 2H), 7.31 (td, J=7.7, 2.4 Hz, 1H), 5.92 (d, J=10.1 Hz, 1H), 4.53 (td, J=9.5, 4.7 Hz, 1H), 3.10-2.57 (m, 2H), 1.12 (s, 9H). ¹⁹F NMR (282 MHz, DMSO) δ −61.36.

### Step-3: Preparation of ethyl 2-(2-((7-(3-(1-((R)-1,1-dimethylethylsulfinamido)-3,3,3-trifluoropropyl)phenyl)benzofuran-5-yl)methoxy)phenyl)acetate (41d)

Compound 41d was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (366 mg, 0.838 mmol) in dioxane (5 mL) using (R)—N-(1-(3-bromophenyl)-3,3,3-trifluoropropyl)-2-methylpropane-2-sulfinamide (41c) (260 mg, 0.698 mmol), bis(triphenylphosphine)Palladium(II)chloride [Pd(PPh₃)₂Cl₂](49.0 mg, 0.070 mmol), 3.3 M aqueous K₂CO₃ (0.635 mL, 2.095 mmol) and heating at 100° C. for 16 h on oil bath. This gave after workup and purification by flash column chromatography [silica gel (24 g), eluting with 0-3% MeOH in DCM]ethyl 2-(2-((7-(3-(1-((R)-1,1-dimethylethylsulfinamido)-3,3,3-trifluoropropyl)phenyl)benzofuran-5-yl)methoxy)phenyl)acetate (41d) (248 mg, 59.0% yield) as orange oils, which were used as such in the next reaction; MS (ES+) 602 (M+1).

### Step-4: Preparation of 2-(2-((7-(3-(1-amino-3,3,3-trifluoropropyl)phenyl)benzofuran-5-yl)methoxy)phenyl)acetic acid (41e)

To a solution of ethyl 2-(2-((7-(3-(1-((R)-1,1-dimethylethylsulfinamido)-3,3,3-trifluoropropyl)phenyl)benzofuran-5-yl)methoxy)phenyl)acetate (41d) (248 mg, 0.412 mmol) in EtOH (5 mL) was added 4 M HCl in dioxane (0.206 mL, 0.824 mmol) and stirred at rt for 1 h. To the reaction was added 2 M aqueous LiOH (0.561 mL, 1.122 mmol) and stirred at RT for 16 h neutralized with 3 M aqueous HCl to pH 7. The neutral solution was purified by reverse-phase column chromatography [C-18 column (100 g), eluting with 0.1% aqueous HCl in H₂O and MeCN from 0-100%] to provide 2-(2-((7-(3-(1-amino-3,3,3-trifluoropropyl)phenyl)benzofuran-5-yl)methoxy)phenyl)acetic acid (41e) (135 mg, 77% yield) HCl salt as a white solid. ¹H NMR (300 MHz, DMSO-d₆) δ 8.05 (d, J=2.2 Hz, 2H), 7.93 (dt, J=7.2, 1.7 Hz, 1H), 7.70 (d, J=1.6 Hz, 1H), 7.66-7.50 (m, 3H), 7.17 (t, J=7.6 Hz, 2H), 7.03 (d, J=8.1 Hz, 1H), 6.99 (d, J=2.2 Hz, 1H), 6.84 (t, J=7.4 Hz, 1H), 5.21 (s, 2H), 4.66 (dd, J=8.9, 5.5 Hz, 1H), 3.53 (s, 2H), 3.14 (d, J=10.0 Hz, 2H). ¹⁹F NMR (282 MHz, DMSO-d₆) δ −61.80; MS (ES+): 470.0 (M+1), (ES−): 467.0 (M−1); Analysis calculated for C₂₆H₂₂F₃NO₄·HCl·1.25H₂O: C, 59.10; H, 4.86; Cl, 6.71; N, 2.65; Found: C, 59.11; H, 4.67; Cl, 6.75; N, 2.78.

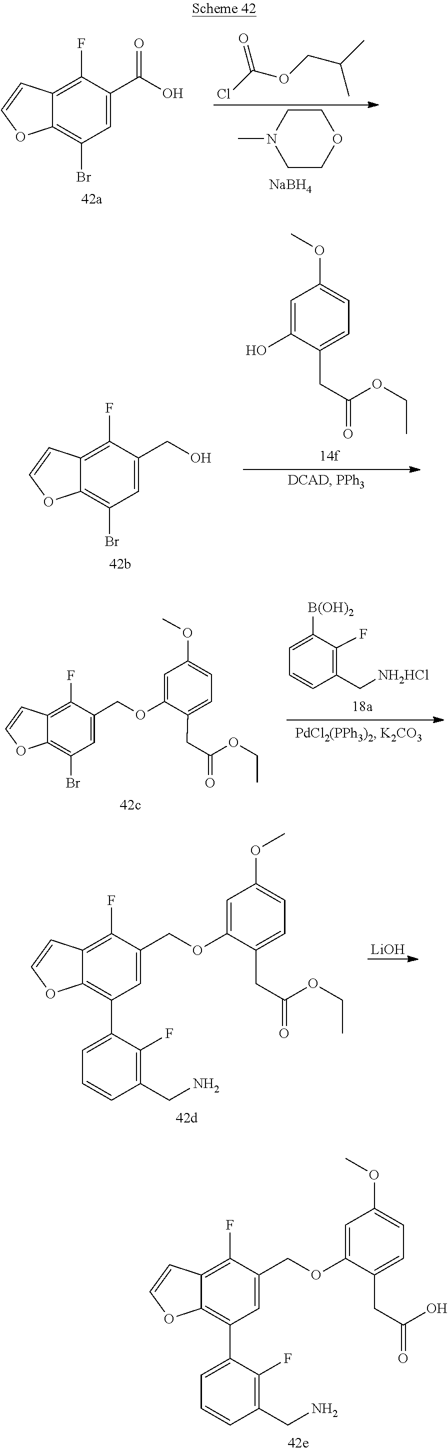

### Preparation of 2-(2-((7-(3-(aminomethyl)-2-fluorophenyl)-4-fluorobenzofuran-5-yl)methoxy)-4-methoxyphenyl)acetic acid (42e)

### Step-1: Preparation of (7-bromo-4-fluorobenzofuran-5-yl)methanol (42b)

Compound 42b was prepared according to the procedure reported in step-1 of scheme 1 from 7-bromo-4-fluorobenzofuran-5-carboxylic acid (42a) (900 mg, 3.47 mmol, purchased from PharmaBlock, PB95207) using N-methylmorpholine (0.44 mL, 3.97 mmol) in THE (10 mL), isobutyl chloroformate (0.55 mL, 4.17 mmol) and NaBH₄ (394 mg, 10.42 mmol) in water (5 mL). This gave after workup and purification by flash chromatography [silica (12g), eluting with EtOAc in hexane from 0-60%](7-bromo-4-fluorobenzofuran-5-yl)methanol (42b) (760 mg, 89% yield) as a white solid; ¹H NMR (300 MHz, DMSO-d₆) δ 8.17 (dt, J=2.2, 0.5 Hz, 1H), 7.60 (dt, J=6.4, 0.6 Hz, 1H), 7.21 (d, J=2.2 Hz, 1H), 5.39 (t, J=5.8 Hz, 1H), 4.65-4.57 (m, 2H); ¹⁹F NMR (282 MHz, DMSO-d₆) δ −126.62.

### Step-2: Preparation of ethyl 2-(2-((7-bromo-4-fluorobenzofuran-5-yl)methoxy)-4-methoxyphenyl)acetate (42c)

Compound 42c was prepared according to the procedure reported in step-2 of scheme 1 from (7-bromo-4-fluorobenzofuran-5-yl)methanol (42b) (594 mg, 2.424 mmol) in DCM (75 mL) using triphenylphosphine (699 mg, 2.67 mmol), ethyl 2-(2-hydroxy-4-methoxyphenyl)acetate (14f) (612 mg, 2.91 mmol) and (E)-bis(4-chlorobenzyl) diazene-1,2-dicarboxylate (DCAD) (979 mg, 2.67 mmol) in DCM (20 mL). This gave after workup and purification by flash column chromatography [silica (24 g), eluting with EtOAc in hexane from 0-50%]ethyl 2-(2-((7-bromo-4-fluorobenzofuran-5-yl)methoxy)-4-methoxyphenyl)acetate (42c) (703 mg, 66% yield) as a white solid. ¹H NMR (300 MHz, DMSO-d₆) δ 8.22 (d, J=2.2 Hz, 1H), 7.70 (d, J=6.1 Hz, 1H), 7.28 (d, J=2.3 Hz, 1H), 7.11 (d, J=8.3 Hz, 1H), 6.74 (d, J=2.4 Hz, 1H), 6.51 (dd, J=8.3, 2.4 Hz, 1H), 5.20 (d, J=1.4 Hz, 2H), 3.95 (q, J=7.1 Hz, 2H), 3.76 (s, 3H), 3.48 (s, 2H), 1.01 (t, J=7.1 Hz, 3H); ¹⁹F NMR (282 MHz, DMSO-d₆) δ −124.53.

### Step-3: Preparation of ethyl 2-(2-((7-(3-(aminomethyl)-2-fluorophenyl)-4-fluorobenzofuran-5-yl)methoxy)-4-methoxyphenyl)acetate (42d)

Compound 42d was prepared according to procedure reported in Step-4 of scheme 1 from ethyl 2-(2-((7-bromo-4-fluorobenzofuran-5-yl)methoxy)-4-methoxyphenyl)acetate (42c) (150 mg, 0.343 mmol) in dioxane (5 mL) using (3-(aminomethyl)-2-fluorophenyl)boronic acid hydrochloride (18a) (87 mg, 0.515 mmol), bis(triphenylphosphine)Palladium(II)chloride [Pd(PPh₃)₂Cl₂](36.1 mg, 0.051 mmol) a solution of K₂CO₃ (142 mg, 1.029 mmol) in water (0.5 mL) and heating at 100° C. for 3 h on oil bath. This gave after workup and purification by flash column chromatography [silica gel (12 g), eluting with DMA80 in DCM from 0-70%]ethyl 2-(2-((7-(3-(aminomethyl)-2-fluorophenyl)-4-fluorobenzofuran-5-yl)methoxy)-4-methoxyphenyl)acetate (42d) (165 mg, 100% yield) as a dark oil. MS (ES+): 482.2 (M+1).

### Step-4: Preparation of 2-(2-((7-(3-(aminomethyl)-2-fluorophenyl)-4-fluorobenzofuran-5-yl)methoxy)-4-methoxyphenyl)acetic acid (42e)

Compound 42e was prepared according to procedure reported in Step-8 of scheme 1 from ethyl 2-(2-((7-(3-(aminomethyl)-2-fluorophenyl)-4-fluorobenzofuran-5-yl)methoxy)-4-methoxyphenyl)acetate (42d) (163 mg, 0.339 mmol) in THE (6 mL), acetonitrile (6 mL) using a solution of lithium hydroxide monohydrate (86 mg, 2.050 mmol) in water (2 mL) and stirring overnight at room temperature. This gave after workup and purification by reverse-phase column chromatography [C-18 column, eluting with 0.1% aq HCl in water and acetonitrile from 0-100%]2-(2-((7-(3-(aminomethyl)-2-fluorophenyl)-4-fluorobenzofuran-5-yl)methoxy)-4-methoxyphenyl)acetic acid (42e) (55 mg, 36% yield) HCl salt as a white solid. ¹H NMR (300 MHz, DMSO-d₆) δ 8.72 (s, 1H), 8.14 (d, J=2.3 Hz, 1H), 7.77-7.61 (m, 2H), 7.58 (d, J=6.7 Hz, 1H), 7.43 (t, J=7.7 Hz, 1H), 7.23 (d, J=2.3 Hz, 1H), 7.10 (d, J=8.3 Hz, 1H), 6.75 (d, J=2.4 Hz, 1H), 6.50 (dd, J=8.3, 2.4 Hz, 1H), 5.28 (s, 2H), 4.17 (s, 2H), 3.75 (s, 3H), 3.43 (s, 2H); ¹⁹F NMR (282 MHz, DMSO) δ −118.46, −123.53; MS (ES+): 454.1 (M+1); MS (ES−): 452.1 (M−1); Analysis calculated for C₂₅H₂₁F₂NO₅·0.95HCl·1.5H₂O: C, 58.29; H, 4.88; Cl, 6.54; N, 2.72; Found: C, 57.91; H, 4.82; Cl, 6.44; N, 2.61.

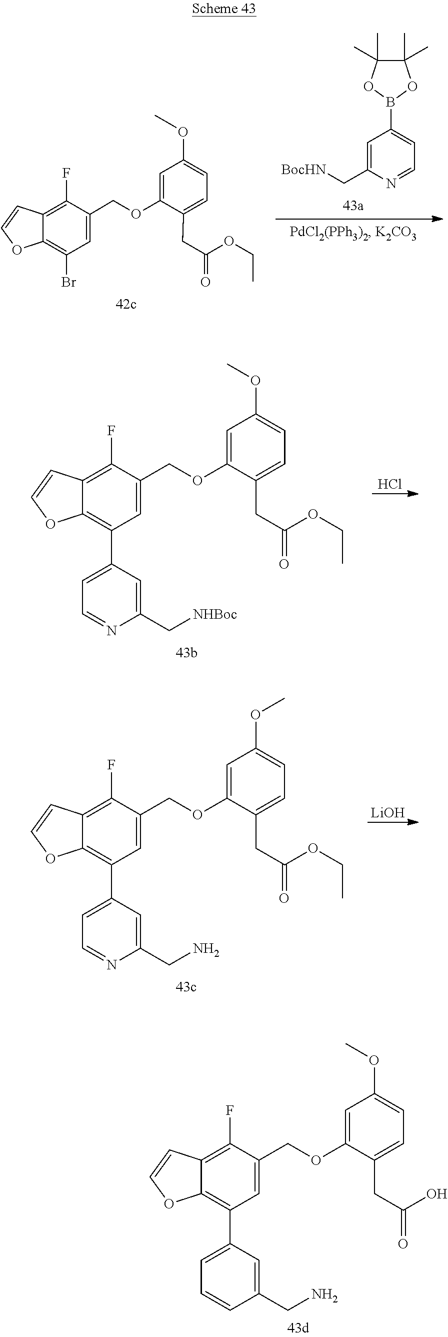

### Preparation of 2-(2-((7-(2-(aminomethyl)pyridin-4-yl)-4-fluorobenzofuran-5-yl)methoxy)-4-methoxyphenyl)acetic acid (43d)

### Step-1: Preparation of ethyl 2-(2-((7-(2-((tert-butoxycarbonylamino)methyl)pyridin-4-yl)-4-fluorobenzofuran-5-yl)methoxy)-4-methoxyphenyl)acetate (43b)

Compound 43b was prepared according to procedure reported in Step-4 of scheme 1 from ethyl 2-(2-((7-bromo-4-fluorobenzofuran-5-yl)methoxy)-4-methoxyphenyl)acetate (42c) (137 mg, 0.313 mmol) in dioxane (5 mL) using tert-butyl (4-(4,4,5,5-tetramethyl-1,3,2-dioxaborolan-2-yl)pyridin-2-yl)methylcarbamate (43a) (136 mg, 0.407 mmol; CAS #1425334-54-8), bis(triphenylphosphine)Palladium(II)chloride [Pd(PPh₃)₂Cl₂](33.0 mg, 0.047 mmol), a solution of K₂CO₃ (130 mg, 0.940 mmol) in water (0.5 mL) and heating at 100° C. for 3 h on oil bath. This gave after workup and purification by flash column chromatography [silica gel (12 g), eluting with DMA80 in DCM from 0-70%]ethyl 2-(2-((7-(2-((tert-butoxycarbonylamino)methyl)pyridin-4-yl)-4-fluorobenzofuran-5-yl)methoxy)-4-methoxyphenyl)acetate (43b) (177 mg, 0.313 mmol, 100% yield) as a dark oil. MS (ES+): 565.2 (M+1).

### Step-2: Preparation of ethyl 2-(2-((7-(2-(aminomethyl)pyridin-4-yl)-4-fluorobenzofuran-5-yl)methoxy)-4-methoxyphenyl)acetate (43c)

To a solution of ethyl 2-(2-((7-(2-((tert-butoxycarbonylamino)methyl)pyridin-4-yl)-4-fluorobenzofuran-5-yl)methoxy)-4-methoxyphenyl)acetate (43b) (177 mg, 0.313 mmol) in ethanol (6 mL) was added 4 M HCl in dioxanes (0.8 mL, 3.20 mmol) and stirred at RT for 48 h. The solvent was removed in vacuo to afford ethyl 2-(2-((7-(2-(aminomethyl)pyridin-4-yl)-4-fluorobenzofuran-5-yl)methoxy)-4-methoxyphenyl)acetate (43c) (146 mg, 100% yield) as a yellow solid. MS (ES+): 465.2 (M+1).

### Step-3: Preparation of 2-(2-((7-(2-(aminomethyl)pyridin-4-yl)-4-fluorobenzofuran-5-yl)methoxy)-4-methoxyphenyl)acetic acid (43d)

Compound 43d was prepared according to procedure reported in Step-8 of scheme 1 from ethyl 2-(2-((7-(2-(aminomethyl)pyridin-4-yl)-4-fluorobenzofuran-5-yl)methoxy)-4-methoxyphenyl)acetate (43c) (146 mg, 0.314 mmol) in THE (6 mL), acetonitrile (6 mL) using a solution of lithium hydroxide monohydrate (135 mg, 3.22 mmol) in water (2 mL) and stirring overnight at room temperature. This gave after workup and purification by reverse-phase column chromatography [C-18 column, eluting with 0.1% aq HCl in water and acetonitrile from 0-100%]2-(2-((7-(2-(aminomethyl)pyridin-4-yl)-4-fluorobenzofuran-5-yl)methoxy)-4-methoxyphenyl)acetic acid (43d) (105 mg, 77% yield) HCl salt as a white solid. ¹H NMR (300 MHz, DMSO-d₆) δ 8.78 (d, J=5.3 Hz, 1H), 8.60 (s, 3H), 8.25 (d, J=2.3 Hz, 1H), 8.10 (s, 1H), 8.04-7.87 (m, 2H), 7.28 (d, J=2.3 Hz, 1H), 7.12 (d, J=8.3 Hz, 1H), 6.76 (d, J=2.4 Hz, 1H), 6.51 (dd, J=8.3, 2.3 Hz, 1H), 5.31 (s, 2H), 4.31 (d, J=5.3 Hz, 2H), 3.76 (s, 3H), 3.48 (s, 2H); ¹⁹F NMR (282 MHz, DMSO) δ −121.51; MS (ES+): 437.1 (M+1); MS (ES−): 435.1 (M−1); Analysis calculated for C₂₄H₂₁FN₂O₅·1.35HCl·2.5H₂O: C, 54.32; H, 5.19; Cl, 9.02; N, 5.28; Found: C, 54.18; H, 5.13; Cl, 9.24; N, 5.28.

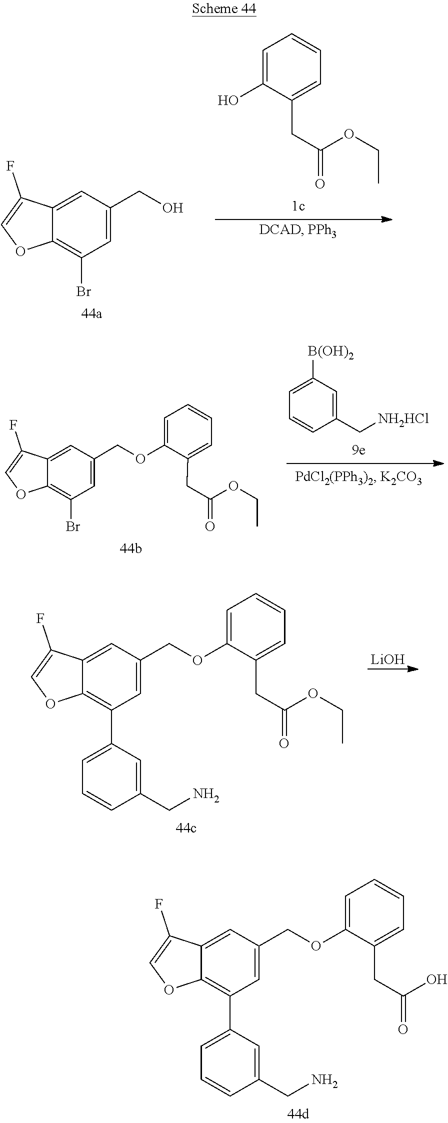

### Preparation of 2-(2-((7-(3-(aminomethyl)phenyl)-3-fluorobenzofuran-5-yl)methoxy)phenyl)acetic acid (44d)

### Step-1: Preparation of ethyl 2-(2-((7-bromo-3-fluorobenzofuran-5-yl)methoxy)phenyl)acetate (44b)

Compound 44b was prepared according to the procedure reported in step-2 of scheme 1 from (7-bromo-3-fluorobenzofuran-5-yl)methanol (44a) (PharmaBlock, Cat #: PB98116, 202 mg, 0.824 mmol) in DCM (6 mL) using triphenylphosphine (281 mg, 1.072 mmol), ethyl 2-(2-hydroxyphenyl)acetate (1c) (193 mg, 1.072 mmol) and (E)-bis(4-chlorobenzyl) diazene-1,2-dicarboxylate (DCAD, 393 mg, 1.072 mmol) in DCM (2 mL). This gave after workup and purification by flash column chromatography [silica (12 g), eluting with EtOAc in hexane from 0-50%]ethyl 2-(2-((7-bromo-3-fluorobenzofuran-5-yl)methoxy)phenyl)acetate (44b) (196 mg, 58% yield) as a white solid. ¹H NMR (300 MHz, DMSO-d₆) δ 8.46 (d, J=4.4 Hz, 1H), 7.75 (dd, J=12.2, 1.5 Hz, 2H), 7.31-7.17 (m, 2H), 7.07 (d, J=8.1 Hz, 1H), 6.93 (td, J=7.3, 1.1 Hz, 1H), 5.22 (s, 2H), 4.03 (q, J=7.1 Hz, 2H), 3.64 (s, 2H), 1.09 (t, J=7.1 Hz, 3H); ¹⁹F NMR (282 MHz, DMSO) δ −174.46.

### Step-2: Preparation of ethyl 2-(2-((7-(3-(aminomethyl)phenyl)-3-fluorobenzofuran-5-yl)methoxy)phenyl)acetate (44c)

Compound 44c was prepared according to procedure reported in Step-4 of scheme 1 from ethyl 2-(2-((7-bromo-3-fluorobenzofuran-5-yl)methoxy)phenyl)acetate (44b) (155 mg, 0.381 mmol) in dioxane (5 mL) using 3-(aminomethyl)phenylboronic acid hydrochloride (9e) (86 mg, 0.571 mmol), bis(triphenylphosphine)Palladium(II)chloride [Pd(PPh₃)₂Cl₂](40.1 mg, 0.057 mmol), a solution of K₂CO₃ (158 mg, 1.142 mmol) in water (0.5 mL) and heating at 100° C. for 3 h on oil bath. This gave after workup and purification by flash column chromatography [silica gel (12 g), eluting with DMA-80 in DCM from 0-70%]ethyl 2-(2-((7-(3-(aminomethyl)phenyl)-3-fluorobenzofuran-5-yl)methoxy)phenyl)acetate (44c) (146 mg, 88% yield) as a dark oil. ¹H NMR (300 MHz, DMSO-d₆) δ 8.37 (d, J=4.4 Hz, 1H), 7.84-7.77 (m, 1H), 7.75-7.65 (m, 3H), 7.51-7.38 (m, 2H), 7.31-7.19 (m, 2H), 7.15-7.07 (m, 1H), 6.92 (td, J=7.4, 1.1 Hz, 1H), 5.28 (s, 2H), 3.94 (q, J=7.1 Hz, 2H), 3.81 (s, 2H), 3.65 (s, 2H), 1.01 (t, J=7.1 Hz, 3H); ¹⁹F NMR (282 MHz, DMSO) δ −176.97; MS (ES+): 434.1 (M+1).

### Step-3: Preparation of 2-(2-((7-(3-(aminomethyl)phenyl)-3-fluorobenzofuran-5-yl)methoxy)phenyl)acetic acid (44d)

Compound 44d was prepared according to procedure reported in Step-8 of scheme 1 from ethyl 2-(2-((7-(3-(aminomethyl)phenyl)-3-fluorobenzofuran-5-yl)methoxy)phenyl)acetate (44c) (143 mg, 0.330 mmol) in THE (6 mL), acetonitrile (6 mL) using a solution of lithium hydroxide monohydrate (72 mg, 1.716 mmol) in water (2 mL) and stirring at room temperature for 40 h. This gave after workup and purification by reverse-phase column chromatography [C-18 column, eluting with 0.1% aq HCl in water and acetonitrile from 0-100%]2-(2-((7-(3-(aminomethyl)phenyl)-3-fluorobenzofuran-5-yl)methoxy)phenyl)acetic acid (44d) (101 mg, 76% yield) as a white solid. ¹H NMR (300 MHz, DMSO-d₆) δ 11.98 (s, 1H), 8.60 (s, 2H), 8.46-8.29 (m, 1H), 8.01 (s, 1H), 7.96-7.85 (m, 1H), 7.78 (d, J=9.8 Hz, 2H), 7.69-7.48 (m, 2H), 7.33-7.17 (m, 2H), 7.09 (d, J=8.2 Hz, 1H), 6.92 (t, J=7.4 Hz, 1H), 5.31 (s, 2H), 4.13 (s, 2H), 3.62 (s, 2H); ¹⁹F NMR (282 MHz, DMSO) δ −176.63. MS (ES+): 406.1 (M+1); MS(ES−): 404.1 (M−1); Analysis calculated for C₂₄H₂₀FNO₄·HCl·H₂O: C, 62.68; H, 5.04; Cl, 7.71; N, 3.05; Found: C, 63.03; H, 5.02; Cl, 7.59; N, 3.09.

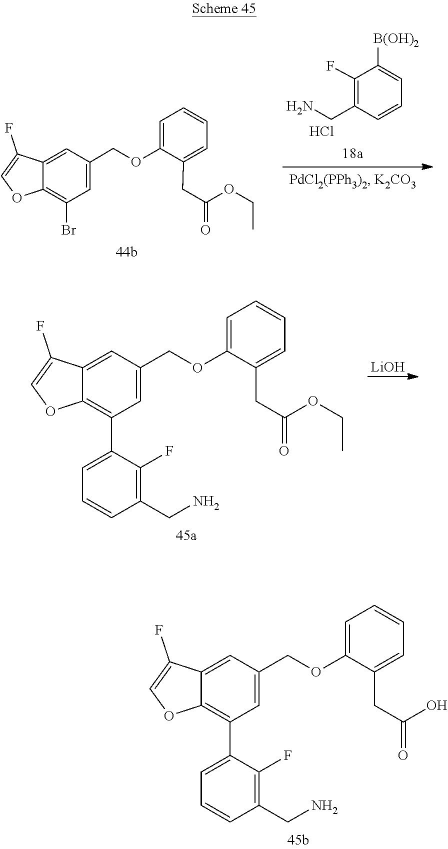 [SMILES: CCOC(=O)Cc1ccccc1OCc1cc(Br)c2occ(F)c2c1 ; NCc1cccc(B(O)O)c1F ; Cl ; CCOC(=O)Cc1ccccc1OCc1cc(-c2cccc(CN)c2F)c2occ(F)c2c1 ; "LiOH" ; NCc1cccc(-c2cc(COc3ccccc3CC(=O)O)cc3c(F)coc23)c1F]

### Preparation of 2-(2-((7-(3-(aminomethyl)-2-fluorophenyl)-3-fluorobenzofuran-5-yl)methoxy)phenyl)acetic acid (45b)

### Step-1: Preparation of ethyl 2-(2-((7-(3-(aminomethyl)-2-fluorophenyl)-3-fluorobenzofuran-5-yl)methoxy)phenyl)acetate (45a)

Compound 45a was prepared according to procedure reported in Step-4 of scheme 1 from ethyl 2-(2-((7-bromo-3-fluorobenzofuran-5-yl)methoxy)phenyl)acetate (44b) (146 mg, 0.359 mmol) in dioxane (5 mL) using (3-(aminomethyl)-2-fluorophenyl)boronic acid hydrochloride (18a) (91 mg, 0.538 mmol), bis(triphenylphosphine)Palladium(II)chloride [Pd(PPh₃)₂Cl₂](37.7 mg, 0.054 mmol), a solution of K₂CO₃ (149 mg, 1.076 mmol) in water (0.5 mL) and heating at 100° C. for 3 h on oil bath. This gave after workup and purification by flash column chromatography [silica gel (12 g), eluting with DMA-80 in DCM from 0-70%]ethyl 2-(2-((7-(3-(aminomethyl)-2-fluorophenyl)-3-fluorobenzofuran-5-yl)methoxy)phenyl)acetate (45a) (149 mg, 92% yield) as a dark oil. ¹H NMR (300 MHz, DMSO-d₆) δ 8.33 (d, J=4.4 Hz, 1H), 7.79 (d, J=1.6 Hz, 1H), 7.67-7.50 (m, 4H), 7.45 (td, J=7.3, 1.9 Hz, 1H), 7.38-7.17 (m, 3H), 7.12 (d, J=8.1 Hz, 1H), 6.92 (td, J=7.4, 1.1 Hz, 1H), 5.27 (s, 2H), 3.92 (q, J=7.1 Hz, 2H), 3.83 (s, 2H), 3.63 (s, 2H), 1.00 (t, J=7.1 Hz, 3H); ¹⁹F NMR (282 MHz, DMSO) δ −121.74, −176.64. MS (ES+): 452.1 (M+1).

### Step-2: Preparation of 2-(2-((7-(3-(aminomethyl)-2-fluorophenyl)-3-fluorobenzofuran-5-yl)methoxy)phenyl)acetic acid (45b)

Compound 45b was prepared according to procedure reported in Step-8 of scheme 1 from ethyl 2-(2-((7-(3-(aminomethyl)-2-fluorophenyl)-3-fluorobenzofuran-5-yl)methoxy)phenyl)acetate (45a) (146 mg, 0.323 mmol) in THE (6 mL), acetonitrile (6 mL) using a solution of lithium hydroxide monohydrate (91 mg, 2.169 mmol) in water (2 mL) and stirring overnight at room temperature. This gave after workup and purification by reverse-phase column chromatography [C-18 column, eluting with 0.1% aq HCl in water and acetonitrile from 0-100%]2-(2-((7-(3-(aminomethyl)-2-fluorophenyl)-3-fluorobenzofuran-5-yl)methoxy)phenyl)acetic acid (45b) (102 mg, 75% yield) HCl salt as a white solid. ¹H NMR (300 MHz, DMSO-d₆) δ 12.22 (s, 1H), 8.64 (s, 3H), 8.36 (d, J=4.4 Hz, 1H), 7.87 (s, 1H), 7.82-7.53 (m, 3H), 7.44 (t, J=7.7 Hz, 1H), 7.23 (d, J=7.7 Hz, 2H), 7.09 (d, J=8.2 Hz, 1H), 6.92 (t, J=7.5 Hz, 1H), 5.31 (s, 2H), 4.17 (s, 2H), 3.60 (s, 2H); ¹⁹F NMR (282 MHz, DMSO) δ −118.38, −176.29. MS (ES+): 424.1 (M+1); MS (ES−): 422.1 (M−1); Analysis calculated for C₂₄H₁₉F₂NO₄·HCl·0.75H₂O:C, 60.89; H, 4.58; Cl, 7.49; N, 2.96; Found: C, 61.19; H, 4.64; Cl, 7.68; N, 3.06.

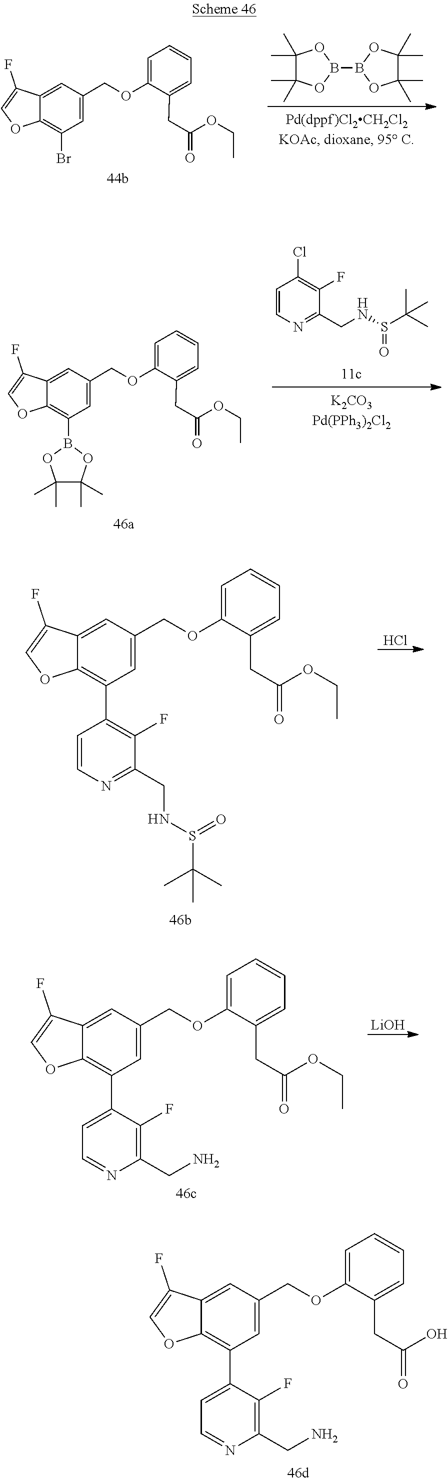

### Preparation of 2-(2-((7-(2-(aminomethyl)-3-fluoropyridin-4-yl)-3-fluorobenzofuran-5-yl)methoxy)phenyl)acetic acid (46d)

### Step-1: Preparation of ethyl 2-(2-((3-fluoro-7-(4,4,5,5-tetramethyl-1,3,2-dioxaborolan-2-yl)benzofuran-5-yl)methoxy)phenyl)acetate (46a)

Compound 46a was prepared according to the procedure reported in step-3 of scheme 1 from ethyl 2-(2-((7-bromo-3-fluorobenzofuran-5-yl)methoxy)phenyl)acetate (44b) (420 mg, 1.031 mmol) using bis(pinacolato)diboron (393 mg, 1.547 mmol), potassium acetate (304 mg, 3.09 mmol) and PdCl₂(dppf)-CH₂Cl₂ (126 mg, 0.155 mmol) in anhydrous dioxane (12 mL) under an nitrogen atmosphere and heating at 95° C. overnight. This gave after workup and purification by flash column chromatography [silica (12 g), eluting with EtOAc in hexane from 0-40%]ethyl 2-(2-((3-fluoro-7-(4,4,5,5-tetramethyl-1,3,2-dioxaborolan-2-yl)benzofuran-5-yl)methoxy)phenyl)acetate (46a) (436 mg, 93% yield) as a white solid. ¹H NMR (300 MHz, DMSO-d₆) δ 8.32 (d, J=4.3 Hz, 1H), 7.86 (d, J=1.8 Hz, 1H), 7.74 (d, J=1.8 Hz, 1H), 7.31-7.17 (m, 2H), 7.14-7.05 (m, 1H), 6.92 (td, J=7.4, 1.1 Hz, 1H), 5.22 (s, 2H), 4.01 (q, J=7.1 Hz, 2H), 3.61 (s, 2H), 1.34 (s, 12H), 1.07 (t, J=7.1 Hz, 3H); ¹⁹F NMR (282 MHz, DMSO) δ −177.50.

### Step-2: Preparation of ethyl 2-(2-((7-(2-((1,1-dimethylethylsulfinamido)methyl)-3-fluoropyridin-4-yl)-3-fluorobenzofuran-5-yl)methoxy)phenyl)acetate (46b)

Compound 46b was prepared according to procedure reported in Step-4 of scheme 1 from ethyl 2-(2-((3-fluoro-7-(4,4,5,5-tetramethyl-1,3,2-dioxaborolan-2-yl)benzofuran-5-yl)methoxy)phenyl)acetate (46a) (219 mg, 0.482 mmol) in dioxane (5 mL) using (+)-N-((4-chloro-3-fluoropyridin-2-yl)methyl)-2-methylpropane-2-sulfinamide (11c) (191 mg, 0.723 mmol), bis(triphenylphosphine)Palladium(II)chloride [Pd(PPh₃)₂Cl₂](50.8 mg, 0.072 mmol), a solution of K₂CO₃ (200 mg, 1.446 mmol) in water (0.5 mL) and heating at 100° C. for 3 h on oil bath. This gave after workup and purification by flash column chromatography [silica gel (12 g), eluting with 0 to 15% methanol in DCM]ethyl 2-(2-((7-(2-((1,1-dimethylethylsulfinamido)methyl)-3-fluoropyridin-4-yl)-3-fluorobenzofuran-5-yl)methoxy)phenyl)acetate (46b) (268 mg, 100% yield) as a dark oil. MS (ES+): 557.2 (M+1).

### Step-3: Preparation of ethyl 2-(2-((7-(2-(aminomethyl)-3-fluoropyridin-4-yl)-3-fluorobenzofuran-5-yl)methoxy)phenyl)acetate (46c)

To a solution of ethyl 2-(2-((7-(2-((1,1-dimethylethylsulfinamido)methyl)-3-fluoropyridin-4-yl)-3-fluorobenzofuran-5-yl)methoxy)phenyl)acetate (46b) (268 mg, 0.481 mmol) in EtOH (6 mL) was added 4M HCl in dioxane (1.5 mL, 6.00 mmol) at 0° C. and allowed to warm to room temperature over a period of 1 h. The reaction mixture was concentrated in vacuo to afford ethyl 2-(2-((7-(2-(aminomethyl)-3-fluoropyridin-4-yl)-3-fluorobenzofuran-5-yl)methoxy)phenyl)acetate (46c) (218 mg, 100% yield) as a yellow oil. MS (ES+): 453.1 (M+1).

### Step-4: Preparation of 2-(2-((7-(2-(aminomethyl)-3-fluoropyridin-4-yl)-3-fluorobenzofuran-5-yl)methoxy)phenyl)acetic acid (46d)

Compound 46d was prepared according to procedure reported in Step-8 of scheme 1 ethyl 2-(2-((7-(2-(aminomethyl)-3-fluoropyridin-4-yl)-3-fluorobenzofuran-5-yl)methoxy)phenyl)acetate (46c) (218 mg, 0.482 mmol) in THF/acetonitrile (6 mL each) using a solution of LiOH (150 mg, 3.57 mmol) in water (2 mL) and stirring at room temperature for 48 h. This gave after workup and purification by reverse-phase column chromatography [C-18 column, eluting with 0.1% aq HCl in water and acetonitrile from 0-100%]2-(2-((7-(2-(aminomethyl)-3-fluoropyridin-4-yl)-3-fluorobenzofuran-5-yl)methoxy)phenyl)acetic acid (46d) (40 mg, 19.56% yield) HCl salt as a white yellow solid. ¹H NMR (300 MHz, DMSO-d₆) δ 12.24 (s, 1H), 8.62 (q, J=6.4, 5.7 Hz, 4H), 8.42 (d, J=4.3 Hz, 1H), 7.96 (d, J=1.6 Hz, 1H), 7.80 (t, J=5.3 Hz, 1H), 7.71 (s, 1H), 7.25 (t, J=7.8 Hz, 2H), 7.09 (d, J=8.1 Hz, 1H), 6.92 (t, J=7.4 Hz, 1H), 5.32 (s, 2H), 4.38 (t, J=5.9 Hz, 2H), 3.60 (s, 2H); ¹⁹F NMR (282 MHz, DMSO) δ −128.26, −176.19. MS (ES+): 425.1 (M+1); MS (ES−): 423.1 (M−1); Analysis calculated for C₂₃H₁₈F₂N₂O₄·1.1HCl·1.4H₂O: C, 56.41; H, 4.51; Cl, 7.96; N, 5.72; Found: C, 56.44; H, 4.16; Cl, 7.73; N, 6.09.

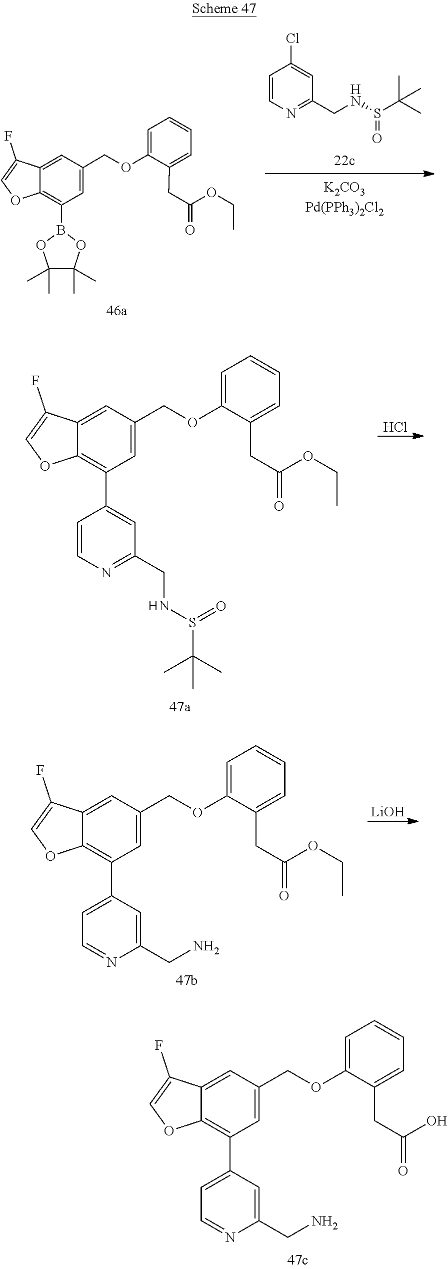

### Preparation of 2-(2-((7-(2-(aminomethyl)pyridin-4-yl)-3-fluorobenzofuran-5-yl)methoxy)phenyl)acetic acid (47c)

### Step-1: Preparation of ethyl 2-(2-((7-(2-((1,1-dimethylethylsulfinamido)methyl)pyridin-4-yl)-3-fluorobenzofuran-5-yl)methoxy)phenyl)acetate (47a)

Compound 47a was prepared according to procedure reported in Step-4 of scheme 1 from ethyl 2-(2-((3-fluoro-7-(4,4,5,5-tetramethyl-1,3,2-dioxaborolan-2-yl)benzofuran-5-yl)methoxy)phenyl)acetate (46a) (199 mg, 0.438 mmol) in dioxane (5 mL) using (+)-N-((4-chloropyridin-2-yl)methyl)-2-methylpropane-2-sulfinamide (22c) (162 mg, 0.657 mmol), bis(triphenylphosphine)Palladium(II)chloride [Pd(PPh₃)₂Cl₂](46.1 mg, 0.066 mmol), a solution of K₂CO₃ (182 mg, 1.314 mmol) in water (0.5 mL) and heating at 100° C. for 3 h on oil bath. This gave after workup and purification by flash column chromatography [silica gel (12 g) eluting with MeOH/DCM from 0-15%]ethyl 2-(2-((7-(2-((1,1-dimethylethylsulfinamido)methyl)pyridin-4-yl)-3-fluorobenzofuran-5-yl)methoxy)phenyl)acetate (47a) (236 mg, 0.438 mmol, 100% yield) as a yellow oil. MS (ES+): 539.2 (M+1).

### Step-2: Preparation of ethyl 2-(2-((7-(2-(aminomethyl)pyridin-4-yl)-3-fluorobenzofuran-5-yl)methoxy)phenyl)acetate (47b)

To a solution of ethyl 2-(2-((7-(2-((1,1-dimethylethylsulfinamido)methyl)pyridin-4-yl)-3-fluorobenzofuran-5-yl)methoxy)phenyl)acetate (47a) (236 mg, 0.438 mmol) in EtOH (6 mL) was added 4 M HCl in dioxane (1.5 mL, 6.00 mmol) at 0° C. and allowed to warm to room temperature over a 1 h period. The reaction mixture was concentrated in vacuo to afford ethyl 2-(2-((7-(2-(aminomethyl)pyridin-4-yl)-3-fluorobenzofuran-5-yl)methoxy)phenyl)acetate (47b) (190 mg, 100% yield) as a yellow solid. MS (ES+): 435.1 (M+1).

### Step-3: Preparation of 2-(2-((7-(2-(aminomethyl)pyridin-4-yl)-3-fluorobenzofuran-5-yl)methoxy)phenyl)acetic acid (47c)

Compound 47c was prepared according to procedure reported in Step-8 of scheme 1 from ethyl 2-(2-((7-(2-(aminomethyl)pyridin-4-yl)-3-fluorobenzofuran-5-yl)methoxy)phenyl)acetate (47b) (190 mg, 0.437 mmol) in THE (6 mL), acetonitrile (6 mL) using a solution of lithium hydroxide monohydrate (132 mg, 3.15 mmol) in water (2 mL) and stirring for 48 h at room temperature. This gave after workup and purification by reverse-phase column chromatography [C-18 column, eluting with 0.1% aq HCl in water and acetonitrile from 0-100%]2-(2-((7-(2-(aminomethyl)pyridin-4-yl)-3-fluorobenzofuran-5-yl)methoxy)phenyl)acetic acid (47c) (67 mg, 38% yield) HCl salt as a white solid. ¹H NMR (300 MHz, DMSO-d₆) δ 8.79 (d, J=5.3 Hz, 1H), 8.57 (s, 3H), 8.47 (d, J=4.3 Hz, 1H), 8.13-8.04 (m, 1H), 8.00-7.94 (m, 1H), 7.92 (s, 2H), 7.32-7.18 (m, 2H), 7.09 (d, J=8.2 Hz, 1H), 6.92 (t, J=7.4 Hz, 1H), 5.33 (s, 2H), 4.31 (d, J=5.6 Hz, 2H), 3.62 (s, 2H); ¹⁹F NMR (282 MHz, DMSO) δ −176.55. MS (ES+): 407.1 (M+1); MS (ES−): 405.1 (M−1); Analysis calculated for C₂₃H₁₉FN₂O₄·1.5HCl·2H₂O: C, 55.57; H, 4.97; Cl, 10.70; N, 5.64; Found: C, 55.52; H, 4.91; Cl, 10.82; N, 5.67.

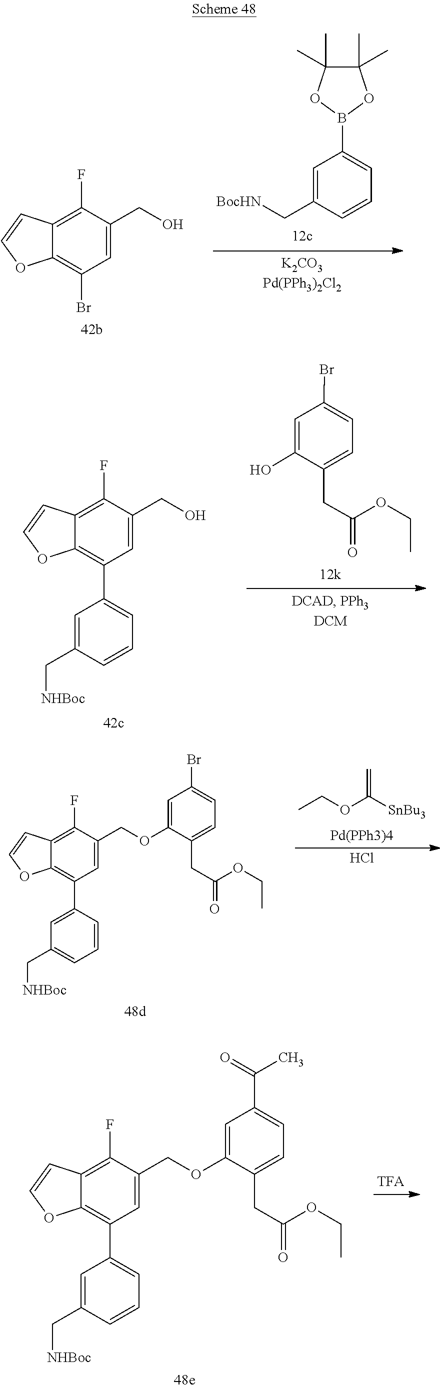

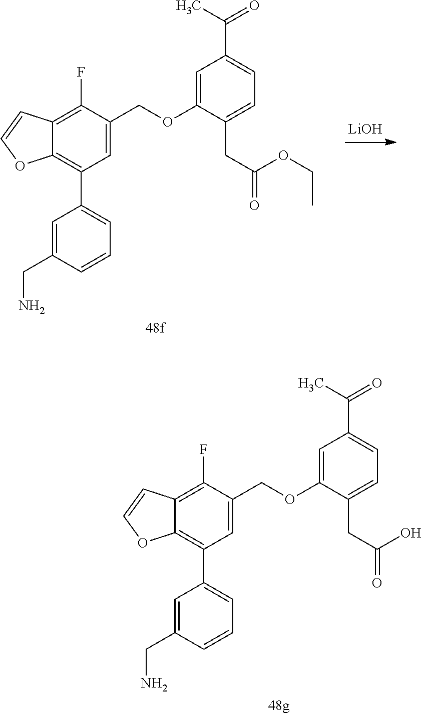 [SMILES: CCOC(=O)Cc1ccc(C(C)=O)cc1OCc1cc(-c2cccc(CN)c2)c2occc2c1F ; "LiOH" ; CC(=O)c1ccc(CC(=O)O)c(OCc2cc(-c3cccc(CN)c3)c3occc3c2F)c1]

### Preparation of 2-(4-acetyl-2-((7-(3-(aminomethyl)phenyl)-4-fluorobenzofuran-5-yl)methoxy)phenyl)acetic acid (48g)

### Step-1: Preparation of tert-butyl 3-(4-fluoro-5-(hydroxymethyl)benzofuran-7-yl)benzylcarbamate (48c)

Compound 48c was prepared according to the procedure reported in step-4 of scheme 1 from (7-bromo-4-fluorobenzofuran-5-yl)methanol (42b) (413 mg, 1.685 mmol) in 1,4-dioxane (8 mL) using tert-butyl 3-(4,4,5,5-tetramethyl-1,3,2-dioxaborolan-2-yl)benzylcarbamate (12c) (674 mg, 2.022 mmol), Pd(PPh₃)₂Cl₂ (177 mg, 0.253 mmol), a solution of K₂CO₃ (699 mg, 5.06 mmol) in water (0.8 mL) and heating at 100° C. for 3 h on an oil bath. This gave after workup, purification by flash column chromatography (silica gel (24 g), eluting with 0-80% EtOAc in hexanes) tert-butyl 3-(4-fluoro-5-(hydroxymethyl)benzofuran-7-yl)benzylcarbamate (48c) (454 mg, 72.5% yield) as a yellow oil. ¹H NMR (300 MHz, DMSO-d₆) δ 8.10 (d, J=2.2 Hz, 1H), 7.73-7.64 (m, 2H), 7.57 (d, J=6.9 Hz, 1H), 7.47 (t, J=7.6 Hz, 2H), 7.29 (d, J=7.6 Hz, 1H), 7.14 (d, J=2.3 Hz, 1H), 5.32 (t, J=5.7 Hz, 1H), 4.68 (dd, J=5.7, 1.5 Hz, 2H), 4.22 (d, J=6.2 Hz, 2H), 1.40 (s, 9H); ¹⁹F NMR (282 MHz, DMSO) δ −127.07.

### Step-2: Preparation of ethyl 2-(4-bromo-2-((7-(3-((tert-butoxycarbonylamino)methyl)phenyl)-4-fluorobenzofuran-5-yl)methoxy)phenyl)acetate (48d)

Compound 48d was prepared according to procedure reported in Step-2 of scheme 1 from tert-butyl 3-(4-fluoro-5-(hydroxymethyl)benzofuran-7-yl)benzylcarbamate (48c) (450 mg, 1.212 mmol) in DCM (6 mL) using triphenylphosphine (413 mg, 1.575 mmol), ethyl 2-(4-bromo-2-hydroxyphenyl)acetate (12k) (471 mg, 1.817 mmol) and a solution of (E)-bis(4-chlorobenzyl) diazene-1,2-dicarboxylate (DCAD) (578 mg, 1.575 mmol) in DCM (2 mL) and stirring at room temperature for 3 h. This gave after workup and purification by flash column chromatography [silica (12g), eluting with EtOAc in hexane from 0-50%]ethyl 2-(4-bromo-2-((7-(3-((tert-butoxycarbonylamino)methyl)phenyl)-4-fluorobenzofuran-5-yl)methoxy)phenyl)acetate (48d) (483 mg, 65.1% yield) as a yellow oil. ¹H NMR (300 MHz, DMSO-d₆) δ 8.16 (d, J=2.3 Hz, 1H), 7.73-7.62 (m, 3H), 7.52-7.42 (m, 3H), 7.30 (d, J=7.6 Hz, 1H), 7.21 (d, J=2.3 Hz, 1H), 7.20-7.11 (m, 2H), 5.30 (s, 2H), 4.22 (d, J=6.1 Hz, 2H), 3.83 (q, J=7.1 Hz, 2H), 3.53 (s, 2H), 1.39 (s, 9H), 0.87 (t, J=7.1 Hz, 3H); ¹⁹F NMR (282 MHz, DMSO) δ −125.01.

### Step-3: Preparation of ethyl 2-(4-acetyl-2-((7-(3-((tert-butoxycarbonylamino)methyl)phenyl)-4-fluorobenzofuran-5-yl)methoxy)phenyl)acetate (48e)

Compound 48e was prepared according to procedure reported in Step-6 of scheme 12 from ethyl 2-(4-bromo-2-((7-(3-((tert-butoxycarbonylamino)methyl)phenyl)-4-fluorobenzofuran-5-yl)methoxy)phenyl)acetate (48d) (480 mg, 0.784 mmol) in toluene (20 mL) using tributyl(1-ethoxyvinyl)stannane (0.349 mL, 0.980 mmol), Pd(Ph3P)4 (91 mg, 0.078 mmol) and heating at 120° C. for 24 h. This gave after workup and purification by flash column chromatography [silica gel (12 g), eluting with ethyl acetate in hexanes from 0-80%]ethyl 2-(4-acetyl-2-((7-(3-((tert-butoxycarbonylamino)methyl)phenyl)-4-fluorobenzofuran-5-yl)methoxy)phenyl)acetate (48e) (240 mg, 53.2% yield) as a pale-yellow oil. ¹H NMR (300 MHz, DMSO-d₆) δ 8.16 (d, J=2.3 Hz, 1H), 7.75-7.63 (m, 4H), 7.59 (dd, J=7.7, 1.5 Hz, 1H), 7.53-7.41 (m, 2H), 7.39 (d, J=7.7 Hz, 1H), 7.30 (d, J=7.6 Hz, 1H), 7.22 (d, J=2.2 Hz, 1H), 5.37 (s, 2H), 4.22 (d, J=6.2 Hz, 2H), 3.85 (q, J=7.1 Hz, 2H), 3.65 (s, 2H), 2.61 (s, 3H), 1.39 (s, 9H), 0.89 (t, J=7.1 Hz, 3H); ¹⁹F NMR (282 MHz, DMSO) δ −125.07.

### Step-4: Preparation of ethyl 2-(4-acetyl-2-((7-(3-(aminomethyl)phenyl)-4-fluorobenzofuran-5-yl)methoxy)phenyl)acetate (48f)

To a solution of ethyl 2-(4-acetyl-2-((7-(3-((tert-butoxycarbonylamino)methyl)phenyl)-4-fluorobenzofuran-5-yl)methoxy)phenyl)acetate (48e) (235 mg, 0.408 mmol) in DCM (5 mL) was added 2,2,2-trifluoroacetic acid (0.629 mL, 8.17 mmol) and stirred at room temperature for 70 min. The reaction mixture was concentrated in vacuum to dryness to furnish ethyl 2-(4-acetyl-2-((7-(3-(aminomethyl)phenyl)-4-fluorobenzofuran-5-yl)methoxy)phenyl)acetate (48f) (194 mg, 100% yield) as a pale-yellow oil. MS (ES+): 476.2 (M+1).

### Step-5: Preparation of 2-(4-acetyl-2-((7-(3-(aminomethyl)phenyl)-4-fluorobenzofuran-5-yl)methoxy)phenyl)acetic acid (48g)

Compound 48g was prepared according to procedure reported in Step-8 of scheme 1 from ethyl 2-(4-acetyl-2-((7-(3-(aminomethyl)phenyl)-4-fluorobenzofuran-5-yl)methoxy)phenyl)acetate (48f) in THE (5 mL), methanol (5 mL) using a solution of lithium hydroxide monohydrate (109 mg, 2.60 mmol) in water (2 mL) and stirring overnight at room temperature. This gave after workup and purification by reverse-phase column chromatography [C-18 column, eluting with 0.1% aq HCl in water and acetonitrile from 0-100%]2-(4-acetyl-2-((7-(3-(aminomethyl)phenyl)-4-fluorobenzofuran-5-yl)methoxy)phenyl)acetic acid (48g) (116 mg, 0.259 mmol, 63.5% yield) HCl salt as a white solid. ¹H NMR (300 MHz, DMSO-d₆) δ 12.29 (s, 1H, D₂O exchangeable), 8.51 (s, 3H, D₂O exchangeable), 8.18 (d, J=2.3 Hz, 1H), 7.98 (s, 1H), 7.94-7.83 (m, 1H), 7.77 (d, J=6.7 Hz, 1H), 7.68 (d, J=1.6 Hz, 1H), 7.63-7.51 (m, 3H), 7.44-7.30 (m, 1H), 7.23 (d, J=2.3 Hz, 1H), 5.40 (s, 2H), 4.12 (s, 2H), 3.64 (s, 2H), 2.60 (s, 3H); ¹H NMR (300 MHz, DMSO-d₆/D₂O) δ 8.05 (d, J=2.2 Hz, 1H), 7.93-7.81 (m, 2H), 7.68 (d, J=6.7 Hz, 1H), 7.58 (dd, J=15.2, 7.4 Hz, 3H), 7.48 (d, J=7.7 Hz, 1H), 7.39-7.29 (m, 1H), 7.15 (d, J=2.2 Hz, 1H), 5.35 (s, 2H), 4.10 (s, 2H), 3.60 (s, 2H), 2.55 (s, 3H); ¹⁹F NMR (282 MHz, DMSO) δ −124.49. MS (ES+): 448.1 (M+1); Analysis calculated for: C₂₆H₂₂FNO₅·1.1HCl·1.25H₂O: C, 61.22; H, 5.06; N, 2.75; Cl, 7.65; Found: C, 61.59; H, 5.04; N, 2.81; Cl, 7.72.

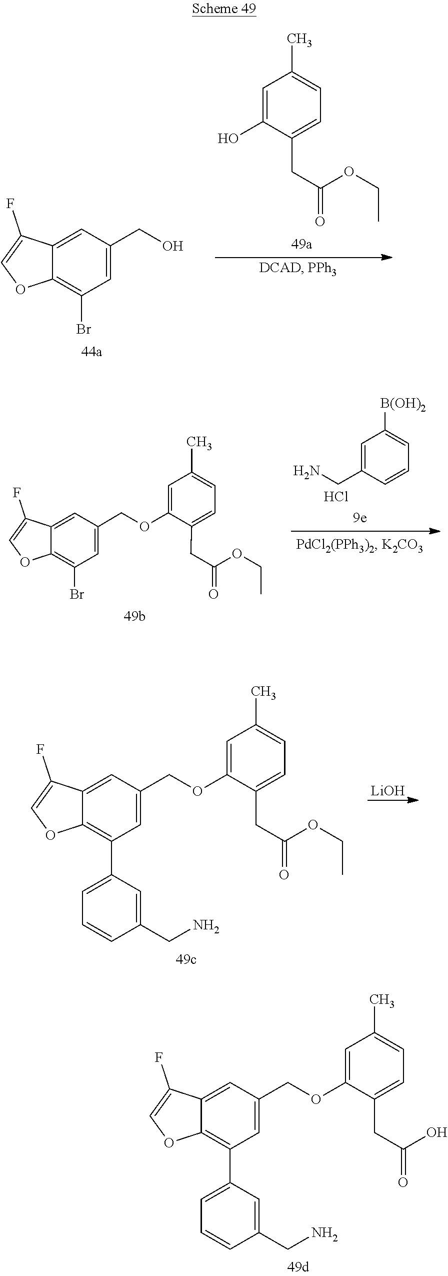

### Preparation of 2-(2-((7-(3-(aminomethyl)phenyl)-3-fluorobenzofuran-5-yl)methoxy)-4-methylphenyl)acetic acid (49d)

### Step-1: Preparation of ethyl 2-(2-((7-bromo-3-fluorobenzofuran-5-yl)methoxy)-4-methylphenyl)acetate (49b)

Compound 49b was prepared according to the procedure reported in step-2 of scheme 1 from (7-bromo-3-fluorobenzofuran-5-yl)methanol (44a) (PharmaBlock, Cat #: PB98116, 202 mg, 0.824 mmol) in DCM (10 mL) using triphenylphosphine (471 mg, 1.796 mmol), ethyl 2-(2-hydroxy-4-methylphenyl)acetate (49a) (CAS #1261451-91-5, 380 mg, 1.959 mmol) and (E)-bis(4-chlorobenzyl) diazene-1,2-dicarboxylate (DCAD) (659 mg, 1.796 mmol) in DCM (5 mL). This gave after workup and purification by flash column chromatography [silica gel (40 g), eluting with EtOAc in hexane from 0-50%]ethyl 2-(2-((7-bromo-3-fluorobenzofuran-5-yl)methoxy)-4-methylphenyl)acetate (49b) (510 mg, 74% yield) as a white solid. ¹H NMR (300 MHz, DMSO-d₆) δ 8.47 (d, J=4.4 Hz, 1H), 7.77 (d, J=1.4 Hz, 1H), 7.72 (d, J=1.5 Hz, 1H), 7.11 (d, J=7.5 Hz, 1H), 6.92 (d, J=1.5 Hz, 1H), 6.75 (d, J=7.5 Hz, 1H), 5.20 (s, 2H), 4.02 (q, J=7.1 Hz, 2H), 3.59 (s, 2H), 2.30 (s, 3H), 1.09 (t, J=7.1 Hz, 3H); ¹⁹F NMR (282 MHz, DMSO) δ −174.41.

### Step-2: Preparation of ethyl 2-(2-((7-(3-(aminomethyl)phenyl)-3-fluorobenzofuran-5-yl)methoxy)-4-methylphenyl)acetate (49c)

Compound 49c was prepared according to procedure reported in Step-4 of scheme 1 from ethyl 2-(2-((7-bromo-3-fluorobenzofuran-5-yl)methoxy)-4-methylphenyl)acetate (49b) (0.5 g, 1.187 mmol) in dioxane (15 mL) using 3-(aminomethyl)phenylboronic acid hydrochloride (9e) (0.267 g, 1.424 mmol), bis(triphenylphosphine)Palladium(II)chloride [Pd(PPh₃)₂Cl₂](0.125 g, 0.178 mmol), a solution of K₂CO₃ (0.492 g, 3.56 mmol) in water (1.5 mL) and heating at 100° C. for 4 h on oil bath. This gave after workup and purification by flash column chromatography [silica gel (40 g), eluting with DMA-80 in DCM from 0-50%]ethyl 2-(2-((7-(3-(aminomethyl)phenyl)-3-fluorobenzofuran-5-yl)methoxy)-4-methylphenyl)acetate (49c) (430 mg, 81% yield) as a clear oil. ¹H NMR (300 MHz, DMSO-d₆) δ 8.39 (d, J=4.3 Hz, 1H), 7.82 (s, 1H), 7.75-7.64 (m, 3H), 7.47 (dt, J=13.8, 7.5 Hz, 2H), 7.10 (d, J=7.5 Hz, 1H), 6.97 (s, 1H), 6.74 (d, J=7.4 Hz, 1H), 5.26 (s, 2H), 3.93 (q, J=7.1 Hz, 2H), 3.83 (s, 2H), 3.59 (s, 2H), 2.31 (s, 3H), 1.00 (t, J=7.1 Hz, 3H); ¹⁹F NMR (282 MHz, DMSO) δ −176.93. MS (ES+): 448.2 (M+1).

### Step-3: Preparation of 2-(2-((7-(3-(aminomethyl)phenyl)-3-fluorobenzofuran-5-yl)methoxy)-4-methylphenyl)acetic acid (49d)

Compound 49d was prepared according to procedure reported in Step-8 of scheme 1 from ethyl 2-(2-((7-(3-(aminomethyl)phenyl)-3-fluorobenzofuran-5-yl)methoxy)-4-methylphenyl)acetate (49c) (181 mg, 0.404 mmol) in THE (6 mL), acetonitrile (6 mL) using a solution of lithium hydroxide monohydrate (118 mg, 2.81 mmol) in water (2 mL) and stirring at room temperature for 16 h. This gave after workup and purification by reverse-phase column chromatography [C-18 column, eluting with 0.1% aq HCl in water and acetonitrile from 0-100%]2-(2-((7-(3-(aminomethyl)phenyl)-3-fluorobenzofuran-5-yl)methoxy)-4-methylphenyl)acetic acid (49d) (28 mg, 17% yield) HCl salt as a white solid. ¹H NMR (300 MHz, DMSO-d₆) δ 12.23 (s, 1H), 8.64-8.42 (m, 3H), 8.39 (d, J=4.3 Hz, 1H), 7.99 (s, 1H), 7.95-7.84 (m, 1H), 7.79 (s, 1H), 7.74 (s, 1H), 7.59 (d, J=4.9 Hz, 2H), 7.10 (d, J=7.6 Hz, 1H), 6.94 (s, 1H), 6.73 (d, J=7.6 Hz, 1H), 5.28 (s, 2H), 4.13 (s, 2H), 3.55 (s, 2H), 2.29 (s, 3H); ¹⁹F NMR (282 MHz, DMSO) δ −176.58; MS (ES+): 420.1 (M+1); MS (ES−): 418.1 (M−1); Analysis calculated for: C₂₅H₂₂FNO₄·HCl·1.75H₂O: C, 61.60; H, 5.48; N, 2.87; Cl, 7.27; Found: C, 61.47; H, 5.40; N, 3.14; Cl, 6.96.

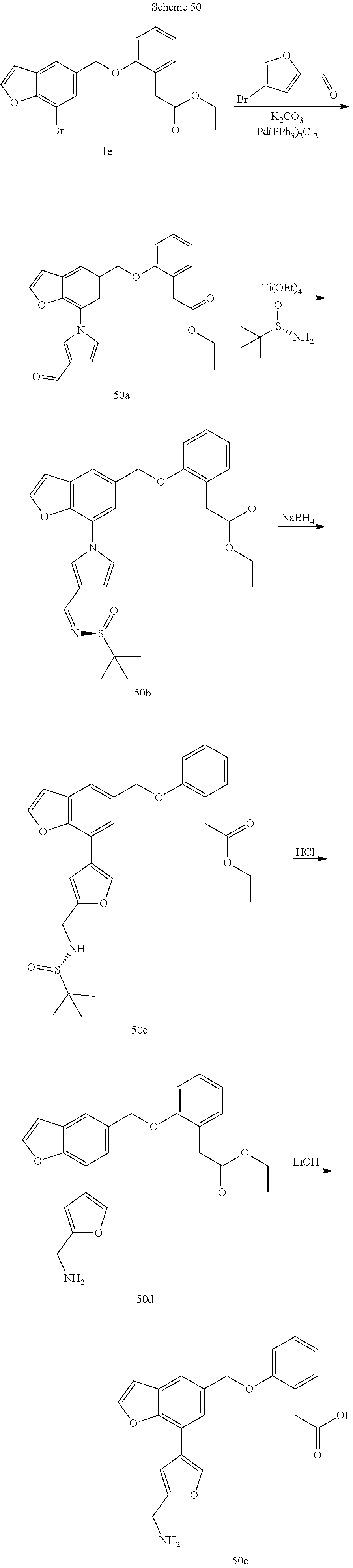

### Preparation of 2-(2-((7-(5-(aminomethyl)furan-3-yl)benzofuran-5-yl)methoxy)phenyl)acetic acid (50e)

### Step-1: Preparation of ethyl 2-(2-((7-(5-formylfuran-3-yl)benzofuran-5-yl)methoxy)phenyl)acetate (50a)

Compound 50a was prepared according to procedure reported in Step-4 of scheme 1 from ethyl 2-(2-((7-(4,4,5,5-tetramethyl-1,3,2-dioxaborolan-2-yl)benzofuran-5-yl)methoxy)phenyl)acetate (1e) (500 mg, 1.146 mmol) in dioxane (6 mL) was added 4-bromofuran-2-carbaldehyde (CAS #21921-76-6; 201 mg, 1.146 mmol), bis(triphenylphosphine)Palladium(II)chloride [Pd(PPh₃)₂Cl₂](121 mg, 0.172 mmol), a solution of K₂CO₃ (475 mg, 3.44 mmol) in water (2 mL) and heated at 90° C. for 3 h. This gave after workup and purification by flash column chromatography [silica gel 12 g, eluting with DMA-80 in DCM from 0-50%]ethyl 2-(2-((7-(5-formylfuran-3-yl)benzofuran-5-yl)methoxy)phenyl)acetate (50a) (400 mg, 86% yield) as a yellow syrup; ¹H NMR (300 MHz, DMSO-d₆) δ 9.74 (d, J=0.8 Hz, 1H), 8.77 (d, J=0.9 Hz, 1H), 8.21 (d, J=0.9 Hz, 1H), 8.15 (d, J=2.2 Hz, 1H), 7.81 (d, J=1.6 Hz, 1H), 7.71 (d, J=1.6 Hz, 1H), 7.30-7.19 (m, 2H), 7.14-7.06 (m, 2H), 6.92 (td, J=7.4, 1.1 Hz, 1H), 5.23 (s, 2H), 3.98 (q, J=7.1 Hz, 2H), 3.66 (s, 2H), 1.01 (t, J=7.1 Hz, 3H).

### Step-2: Preparation of (S)-ethyl 2-(2-((7-(5-(((tert-butylsulfinyl)imino)methyl)furan-3-yl)benzofuran-5-yl)methoxy)phenyl)acetate (50b)

Compound 50b was prepared according to procedure reported in Step-5 of scheme 1 from ethyl 2-(2-((7-(5-formylfuran-3-yl)benzofuran-5-yl)methoxy)phenyl)acetate (50a) (390 mg, 0.964 mmol) in THE (10 mL) using (S)-2-methylpropane-2-sulfinamide (234 mg, 1.929 mmol), tetraethoxytitanium (0.404 mL, 1.929 mmol) and heating at 60° C. for 1 h. This gave after workup and purification by flash column chromatography [(silica gel (24 g), eluting with ethyl acetate in hexanes (0 to 50%)](S)-ethyl 2-(2-((7-(5-(((tert-butylsulfinyl)imino)methyl)furan-3-yl)benzofuran-5-yl)methoxy)phenyl)acetate (50b) (321 mg, 65.6% yield) as a yellow gum; ¹H NMR (300 MHz, DMSO-d₆) δ 8.70 (s, 1H), 8.42 (s, 1H), 8.15 (d, J=2.2 Hz, 1H), 8.03 (d, J=1.0 Hz, 1H), 7.76 (d, J=1.6 Hz, 1H), 7.69 (d, J=1.5 Hz, 1H), 7.31-7.19 (m, 2H), 7.14-7.05 (m, 2H), 6.91 (t, J=7.3 Hz, 1H), 5.23 (s, 2H), 4.00-3.89 (m, 2H), 3.65 (s, 2H), 1.19 (s, 9H), 1.01 (t, J=7.1 Hz, 3H).

### Step-3: Preparation of (S)-ethyl 2-(2-((7-(5-((1,1-dimethylethylsulfinamido)methyl)furan-3-yl)benzofuran-5-yl)methoxy)phenyl)acetate (50c)

Compound 50c was prepared according to procedure reported in Step-3 of scheme 5 from (S)-ethyl 2-(2-((7-(5-(((tert-butylsulfinyl)imino)methyl)furan-3-yl)benzofuran-5-yl)methoxy)phenyl)acetate (50b) (317 mg, 0.625 mmol) in MeOH (10 mL) using sodium borohydride (23.63 mg, 0.625 mmol). This gave after workup and purification by flash column chromatography [(silica gel, 24 g eluting with ethyl acetate in hexanes (0 to 100%)](S)-ethyl 2-(2-((7-(5-((1,1-dimethylethylsulfinamido)methyl)furan-3-yl)benzofuran-5-yl)methoxy)phenyl)acetate (50c) (226 mg, 0.443 mmol, 71.0% yield) as a clear gum; ¹H NMR (300 MHz, DMSO-d₆) δ 8.30 (d, J=0.9 Hz, 1H), 8.10 (d, J=2.2 Hz, 1H), 7.67-7.57 (m, 2H), 7.30-7.19 (m, 2H), 7.13-7.07 (m, 1H), 7.03 (d, J=2.2 Hz, 1H), 7.00 (s, 1H), 6.91 (td, J=7.3, 1.1 Hz, 1H), 5.86 (t, J=5.7 Hz, 1H), 5.21 (s, 2H), 4.22 (dd, J=5.6, 2.3 Hz, 2H), 3.98 (q, J=7.1 Hz, 2H), 3.63 (s, 2H), 1.14 (s, 9H), 1.02 (t, J=7.1 Hz, 3H).

### Step-4: Preparation of ethyl 2-(2-((7-(5-(aminomethyl)furan-3-yl)benzofuran-5-yl)methoxy)phenyl)acetate (50d)

To a solution of (S)-ethyl 2-(2-((7-(5-((1,1-dimethylethylsulfinamido)methyl)furan-3-yl)benzofuran-5-yl)methoxy)phenyl)acetate (50c) (220 mg, 0.432 mmol) in tetrahydrofuran (6 mL) was added aqueous 4N HCl in dioxane (0.216 mL, 0.863 mmol) and stirred at RT for 30 mins. The reaction mixture was concentrated to dryness to furnish ethyl 2-(2-((7-(5-(aminomethyl)furan-3-yl)benzofuran-5-yl)methoxy)phenyl)acetate (50d) which was used in the next step without further purification.

### Step-5: Preparation of 2-(2-((7-(5-(aminomethyl)furan-3-yl)benzofuran-5-yl)methoxy)phenyl)acetic acid (50e)

The residue from above step-4 was dissolved in tetrahydrofuran (4.00 mL), acetonitrile (2 mL), added lithium hydroxide monohydrate (1N aqueous, 2.158 mL, 2.158 mmol) and stirred for 18 h at room temperature. The reaction was concentrated in vacuum diluted with water (2 mL) and acidified to pH 4 using 1M HCl. The solid separated was purified by reverse-phase column chromatography [C-18 column (50 g), eluting with 0.1% aq HCl in water and acetonitrile from 0-100%] to afford 2-(2-((7-(5-(aminomethyl)furan-3-yl)benzofuran-5-yl)methoxy)phenyl)acetic acid (50e) (55 mg, 26.5% yield) HCl salt as a white solid; ¹H NMR (300 MHz, DMSO-d₆) δ 12.23 (s, 1H, D₂O exchangeable), 8.53 (s, 3H, D₂O exchangeable), 8.44 (d, J=0.9 Hz, 1H), 8.12 (d, J=2.2 Hz, 1H), 7.68 (s, 2H), 7.28-7.18 (m, 3H), 7.10-7.01 (m, 2H), 6.90 (td, J=7.4, 1.1 Hz, 1H), 5.25 (s, 2H), 4.18 (d, J=5.3 Hz, 2H), 3.60 (s, 2H). ¹H NMR (300 MHz, DMSO-d₆/D₂O) δ 8.45 (d, J=0.9 Hz, 1H), 8.10 (d, J=2.2 Hz, 1H), 7.68 (q, J=1.7 Hz, 2H), 7.29-7.17 (m, 3H), 7.15-7.00 (m, 2H), 6.98-6.83 (m, 1H), 5.25 (s, 2H), 4.19 (s, 2H), 3.60 (d, J=4.5 Hz, 18H). MS (ES+): 755.2 (2M+1); (ES−): 376.1 (M−1); Analysis calculated for C₂₂H₁₉NO₅·HCl·1.25H₂O: C, 60.55; H, 5.20; Cl, 8.12; N, 3.21; Found: C, 60.57; H, 4.89; Cl, 7.99; N, 3.19.

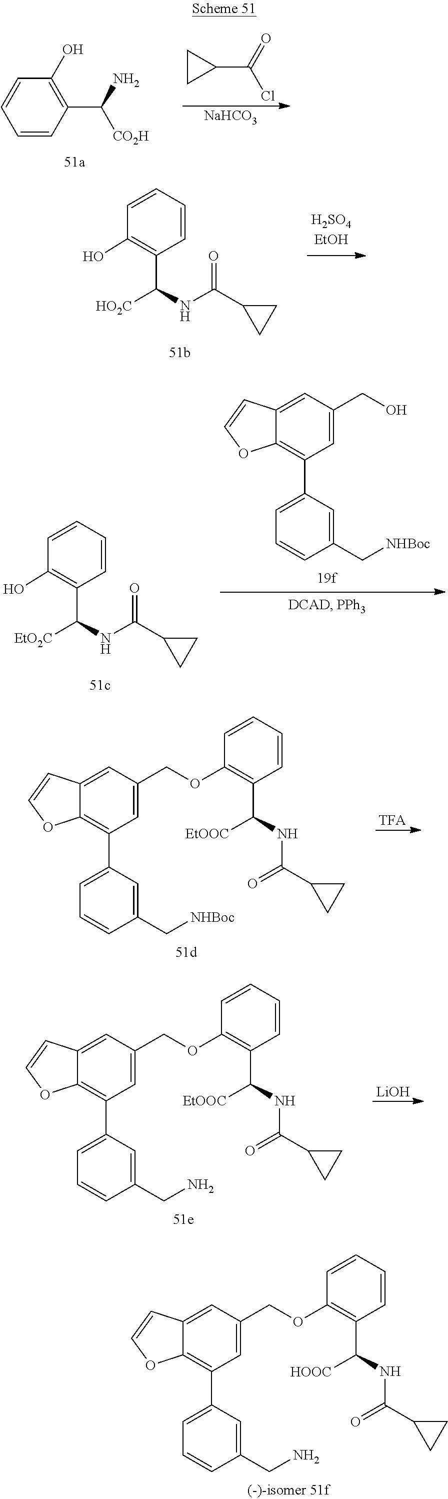

### Preparation of (−)-2-(2-((7-(3-(aminomethyl)phenyl)benzofuran-5-yl)methoxy)phenyl)-2-(cyclopropanecarboxamido)acetic acid (51f)

### Step-1: Preparation of (R)-2-(cyclopropanecarboxamido)-2-(2-hydroxyphenyl)acetic acid (51b)

To a solution of (R)-2-amino-2-(2-hydroxyphenyl)acetic acid (51a) (0.5 g, 2.456 mmol; CAS #185339-06-4) in water (10 mL) cooled to 0° C. was added sodium bicarbonate (0.825 g, 9.82 mmol), a solution of cyclopropanecarbonyl chloride (0.337 mL, 3.68 mmol) in tetrahydrofuran (5 mL) and allowed to warm to RT. The solution was acidified with 3 N HCl and extracted with ethyl acetate (3×50 mL). The combined organic layers were washed with brine (25 mL), dried, filtered and concentrated in vacuum to afford (R)-2-(cyclopropanecarboxamido)-2-(2-hydroxyphenyl)acetic acid (51b) (0.56 g, 97% yield) as a thick syrup, which was used in the next step without further purification; ¹H NMR (300 MHz, DMSO-d6) δ 12.13 (s, 4H), 8.52 (d, J=7.8 Hz, 1H), 7.26-7.05 (m, 2H), 6.93-6.71 (m, 2H), 5.63 (d, J=7.8 Hz, 1H), 1.83-1.71 (m, 1H), 0.70-0.55 (m, 4H).

### Step-2: Preparation of (R)-ethyl 2-(cyclopropanecarboxamido)-2-(2-hydroxyphenyl)acetate (51c)

To a stirred solution of (R)-2-(cyclopropanecarboxamido)-2-(2-hydroxyphenyl)acetic acid (51b) (0.5 g, 2.126 mmol) in ethanol (9 mL) was added H₂SO₄ (0.227 mL, 4.25 mmol) and heated at reflux for 2.5 hrs. The reaction was cooled to room temperature and poured into ice water and extracted with ethyl acetate. The organic layer was washed with sodium bicarbonate, water, brine, dried, filtered and concentrated in vacuum to afford (R)-ethyl 2-(cyclopropanecarboxamido)-2-(2-hydroxyphenyl)acetate (51c) (237 mg, 42.3% yield) as a white solid; ¹H NMR (300 MHz, DMSO-d₆) δ 9.80 (s, 1H), 8.59 (d, J=7.6 Hz, 1H), 7.23-7.09 (m, 2H), 6.89-6.75 (m, 2H), 5.67 (d, J=7.5 Hz, 1H), 4.17-3.91 (m, 2H), 1.84-1.68 (m, 1H), 1.11 (t, J=7.1 Hz, 3H), 0.67 (td, J=7.4, 6.2, 4.4 Hz, 4H).

### Step-3: Preparation (R)-ethyl 2-(2-((7-(3-(((tert-butoxycarbonyl)amino)methyl)phenyl)benzofuran-5-yl)methoxy)phenyl)-2-(cyclopropanecarboxamido)acetate (51d)

Compound 51d was prepared according to the procedure reported in step-2 of scheme 1 from tert-butyl 3-(5-(hydroxymethyl)benzofuran-7-yl)benzylcarbamate (19f) (303 mg, 0.857 mmol) in DCM (10 mL) using triphenylphosphine (246 mg, 0.939 mmol), (R)-ethyl 2-(cyclopropanecarboxamido)-2-(2-hydroxyphenyl)acetate (51c) (215 mg, 0.817 mmol) and a solution of (E)-bis(4-chlorobenzyl) diazene-1,2-dicarboxylate (DCAD) (360 mg, 0.980 mmol) in DCM (7 mL). This gave after workup and purification by flash column chromatography (silica gel (24 g), eluting with 0 to 50% ethyl acetate in hexanes) (R)-ethyl 2-(2-((7-(3-(((tert-butoxycarbonyl)amino)methyl)phenyl)benzofuran-5-yl)methoxy)phenyl)-2-(cyclopropanecarboxamido)acetate (51d) (320 mg, 65.5% yield) as a clear sticky material; MS (ES+): 621.3 (M+Na); ¹H NMR (300 MHz, DMSO-d₆) δ 8.77 (d, J=7.9 Hz, 1H), 8.08 (d, J=2.2 Hz, 1H), 7.80-7.72 (m, 3H), 7.62 (d, J=1.6 Hz, 1H), 7.48 (t, J=7.5 Hz, 2H), 7.37-7.26 (m, 3H), 7.19 (dd, J=8.3, 1.1 Hz, 1H), 7.04 (d, J=2.2 Hz, 1H), 6.99 (td, J=7.5, 1.1 Hz, 1H), 5.87 (d, J=7.9 Hz, 1H), 5.32 (s, 2H), 4.23 (d, J=6.2 Hz, 2H), 3.98 (qd, J=7.1, 5.2 Hz, 2H), 1.75 (td, J=7.5, 3.7 Hz, 1H), 1.40 (s, 9H), 0.98 (t, J=7.1 Hz, 3H), 0.63 (ddt, J=15.3, 13.2, 8.9 Hz, 4H).

### Step-4: Preparation of (R)-ethyl 2-(2-((7-(3-(aminomethyl)phenyl)benzofuran-5-yl)methoxy)phenyl)-2-(cyclopropanecarboxamido)acetate (51e)

To a solution of (R)-ethyl 2-(2-((7-(3-(((tert-butoxycarbonyl)amino)methyl)phenyl)benzofuran-5-yl)methoxy)phenyl)-2-(cyclopropanecarboxamido)acetate (51d) (280 mg, 0.468 mmol) in DCM (3.74 mL) was added 2,2,2-trifluoroacetic acid (0.360 mL, 4.68 mmol), stirred for 2 h at room temperature and concentrated to dryness to afford (R)-ethyl 2-(2-((7-(3-(aminomethyl)phenyl)benzofuran-5-yl)methoxy)phenyl)-2-(cyclopropanecarboxamido)acetate (51e) which was used as such for next step; MS (ES+) 499.20 (M+1).

### Step-5: Preparation of (−)-2-(2-((7-(3-(aminomethyl)phenyl)benzofuran-5-yl)methoxy)phenyl)-2-(cyclopropanecarboxamido)acetic acid (51f)

The residue from step-4 above was dissolved in THE (3.74 mL), acetonitrile (1.87 mL), added lithium hydroxide monohydrate, (1N aqueous solution, 1.871 mL, 1.871 mmol) and stirred for 18 h at room temperature. This gave after work up and purification by reverse phase column [C18 (50 g), eluting with 0.1% aq HCl in water and acetonitrile from 0-100%](−)-2-(2-((7-(3-(aminomethyl)phenyl)benzofuran-5-yl)methoxy)phenyl)-2-(cyclopropanecarboxamido)acetic acid (51f) (95 mg, 43.2% yield) HCl salt as a white solid. ¹H NMR (300 MHz, DMSO-d₆) δ 12.72 (s, 1H, D₂O exchangeable), 8.73 (d, J=8.1 Hz, 1H, D₂O exchangeable), 8.41 (s, 3H, D₂O exchangeable), 8.12 (d, J=2.2 Hz, 1H), 8.02 (s, J=2.0 Hz, 1H), 7.95 (dt, J=7.1, 1.9 Hz, 1H), 7.79 (d, J=1.6 Hz, 1H), 7.71 (d, J=1.7 Hz, 1H), 7.64-7.50 (m, 2H), 7.39-7.26 (m, 2H), 7.17 (d, J=8.1 Hz, 1H), 7.05 (d, J=2.2 Hz, 1H), 6.99 (td, J=7.4, 1.1 Hz, 1H), 5.90 (d, J=8.0 Hz, 1H), 5.33 (s, 2H), 4.15 (s, 2H), 1.77 (dq, J=8.0, 6.0, 5.6 Hz, 1H), 0.77-0.48 (m, 4H); ¹H NMR (300 MHz, DMSO-d₆/D₂O) δ 8.00 (d, J=2.2 Hz, 1H), 7.96-7.87 (m, 2H), 7.76 (d, J=1.6 Hz, 1H), 7.65 (d, J=1.7 Hz, 1H), 7.58 (t, J=7.7 Hz, 1H), 7.48 (d, J=7.7 Hz, 1H), 7.30 (ddd, J=7.3, 4.3, 1.7 Hz, 2H), 7.14 (d, J=8.5 Hz, 1H), 7.05-6.93 (m, 2H), 5.85 (d, J=4.4 Hz, 1H), 5.30 (s, 2H), 4.11 (s, 2H), 1.68 (p, J=6.4 Hz, 1H), 0.62 (dt, J=29.2, 7.1 Hz, 4H); MS (ES+): 471.2 (M+1); Analysis calculated for C₂₈H₂₆N₂O₅·HCl·1.75H₂O: C, 62.45; H, 5.71; Cl, 6.58; N, 5.20; Found: C, 62.68; H, 5.54; Cl, 6.46; N, 5.09; Optical rotation [α]_{D}=−49.091 (c=0.11, MeOH).

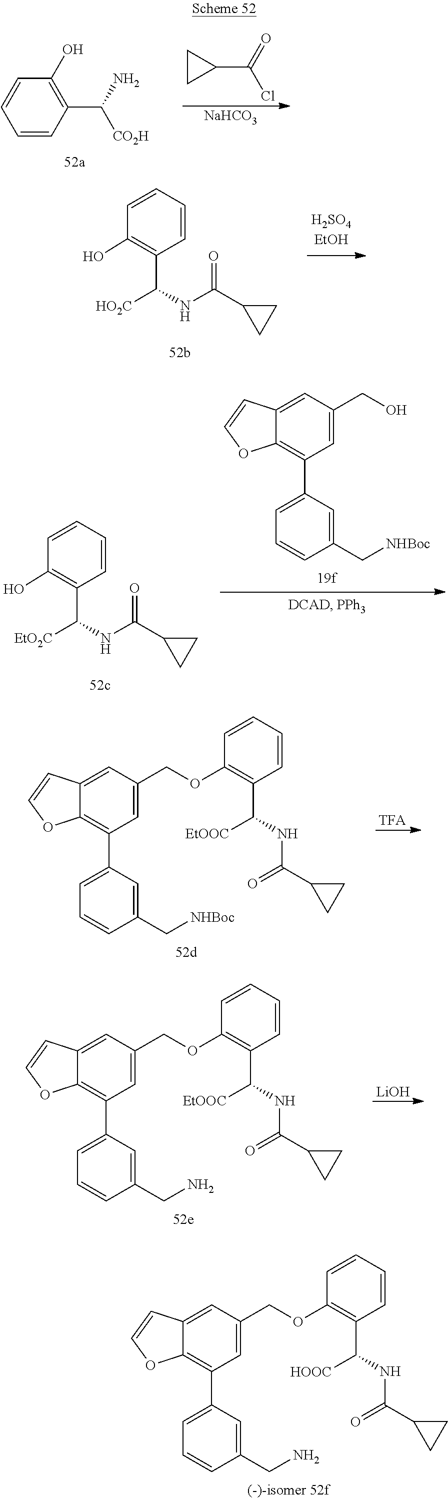

### Preparation of (+)-2-(2-((7-(3-(aminomethyl)phenyl)benzofuran-5-yl)methoxy)phenyl)-2-(cyclopropanecarboxamido)acetic acid (52f)

### Step-1: Preparation of (S)-2-(cyclopropanecarboxamido)-2-(2-hydroxyphenyl)acetic acid (52b)

To a solution of (S)-2-amino-2-(2-hydroxyphenyl)acetic acid (52a) (0.25 g, 1.228 mmol; CAS #185339-08-6) in water (5 mL) cooled to 0° C. was added sodium bicarbonate (0.413 g, 4.91 mmol), a solution of cyclopropanecarbonyl chloride (0.168 mL, 1.842 mmol) in tetrahydrofuran (2.5 mL) and allowed to warm to RT. This gave after workup (S)-2-(cyclopropanecarboxamido)-2-(2-hydroxyphenyl)acetic acid (52b) (233 mg, 81% yield) as a thick syrup. The crude material was used in the next reaction without further purification; ¹H NMR (300 MHz, DMSO-d₆) δ 8.52 (d, J=7.8 Hz, 1H), 7.29-7.06 (m, 2H), 6.81 (ddd, J=16.4, 7.8, 1.2 Hz, 2H), 5.64 (d, J=7.8 Hz, 1H), 1.83-1.72 (m, 1H), 0.65 (dd, J=8.1, 5.2 Hz, 4H).

### Step-2: Preparation of (S)-ethyl 2-(cyclopropanecarboxamido)-2-(2-hydroxyphenyl)acetate (52c)

Compound 52c was prepared according to procedure reported in Step-2 of scheme 51 from (S)-2-(cyclopropanecarboxamido)-2-(2-hydroxyphenyl)acetic acid (52b) (0.22 g, 0.935 mmol) in ethanol (5 mL) using H₂SO₄ (0.100 mL, 1.870 mmol) and heating at reflux for 2.5 h. This gave after workup (S)-ethyl 2-(cyclopropanecarboxamido)-2-(2-hydroxyphenyl)acetate (52c) (97 mg, 0.368 mmol, 39.4% yield) as a white solid; ¹H NMR (300 MHz, DMSO-d₆) δ 9.81 (s, 1H), 8.59 (d, J=7.5 Hz, 1H), 7.15 (ddd, J=7.3, 6.1, 1.9 Hz, 2H), 6.96-6.66 (m, 2H), 5.67 (d, J=7.5 Hz, 1H), 4.07 (qq, J=7.0, 3.8 Hz, 2H), 1.75 (q, J=6.1 Hz, 1H), 1.11 (t, J=7.1 Hz, 3H), 0.66 (t, J=5.9 Hz, 4H).

### Step-3: Preparation (S)-ethyl 2-(2-((7-(3-(((tert-butoxycarbonyl)amino)methyl)phenyl)benzofuran-5-yl)methoxy)phenyl)-2-(cyclopropanecarboxamido)acetate (52d)

Compound 52d was prepared according to procedure reported in Step-3 of scheme 51 from tert-butyl 3-(5-(hydroxymethyl)benzofuran-7-yl)benzylcarbamate (19f) (89 mg, 0.251 mmol) in DCM (5 mL) using triphenylphosphine (78 mg, 0.296 mmol), (S)-ethyl 2-(cyclopropanecarboxamido)-2-(2-hydroxyphenyl)acetate (52c) (60 mg, 0.228 mmol) and a solution of (E)-bis(4-chlorobenzyl) diazene-1,2-dicarboxylate (DCAD) (109 mg, 0.296 mmol) in DCM (3 mL). This gave after workup and purification by flash column chromatography (silica gel (12 g), eluting with 0 to 50% ethyl acetate in hexanes) followed by reverse-phase column chromatography [C-18 column (26 g), eluting with 0.1% aq HCl in water and acetonitrile from 0-100%](S)-ethyl 2-(2-((7-(3-(((tert-butoxycarbonyl)amino)methyl)phenyl)benzofuran-5-yl)methoxy)phenyl)-2-(cyclopropanecarboxamido)acetate (52d) (49 mg, 35.9% yield) as a white solid; ¹H NMR (300 MHz, DMSO-d₆) δ 8.76 (d, J=7.9 Hz, 1H), 8.09 (d, J=2.3 Hz, 1H), 7.75 (d, J=6.7 Hz, 3H), 7.62 (s, 1H), 7.52 (d, J=8.4 Hz, 2H), 7.30 (d, J=7.8 Hz, 3H), 7.19 (d, J=8.3 Hz, 1H), 7.04 (d, J=2.2 Hz, 1H), 7.00 (d, J=7.4 Hz, 1H), 5.87 (d, J=7.8 Hz, 1H), 5.32 (s, 2H), 4.23 (d, J=6.2 Hz, 2H), 4.05-3.91 (m, 2H), 1.77 (d, J=5.2 Hz, 1H), 1.41 (d, J=4.5 Hz, 9H), 0.98 (t, J=7.1 Hz, 3H), 0.62 (dd, J=16.3, 8.7 Hz, 4H).

### Step-4: Preparation of (S)-ethyl 2-(2-((7-(3-(aminomethyl)phenyl)benzofuran-5-yl)methoxy)phenyl)-2-(cyclopropanecarboxamido)acetate (52e)

To a solution of (S)-ethyl 2-(2-((7-(3-(((tert-butoxycarbonyl)amino)methyl)phenyl)benzofuran-5-yl)methoxy)phenyl)-2-(cyclopropanecarboxamido)acetate (52d) (49 mg, 0.082 mmol) in DCM (1.2 mL) was added 2,2,2-trifluoroacetic acid (0.063 mL, 0.818 mmol), stirred for 2 h at room temperature and concentrated to dryness to afford (S)-ethyl 2-(2-((7-(3-(aminomethyl)phenyl)benzofuran-5-yl)methoxy)phenyl)-2-(cyclopropanecarboxamido)acetate (52e) which was used as such for next step; MS (ES+) 499.20 (M+1).

### Step-5: Preparation of (+)-2-(2-((7-(3-(aminomethyl)phenyl)benzofuran-5-yl)methoxy)phenyl)-2-(cyclopropanecarboxamido)acetic acid (52f)

The residue from step-4 above was dissolved in THE (2 mL), acetonitrile (0.6 mL), added lithium hydroxide monohydrate, (1N aqueous solution, 0.327 mL, 0.327 mmol) and stirred for 18 h at room temperature. This gave after work up and purification by reverse phase column [C18 (50 g), eluting with 0.1% aq HCl in water and acetonitrile from 0-100%](+)-2-(2-((7-(3-(aminomethyl)phenyl)benzofuran-5-yl)methoxy)phenyl)-2-(cyclopropanecarboxamido)acetic acid (52f) (35 mg, 91% yield) HCl salt as a white solid; H NMR (300 MHz, DMSO-d₆) δ 12.70 (s, 1H, D₂O exchangeable), 8.72 (d, J=8.1 Hz, 1H, D₂O exchangeable), 8.31 (s, 3H, D₂O exchangeable), 8.12 (d, J=2.2 Hz, 1H), 8.02 (s, 1H), 7.95 (d, J=7.5 Hz, 1H), 7.79 (d, J=1.6 Hz, 1H), 7.71 (s, 1H), 7.57 (dt, J=14.6, 7.6 Hz, 2H), 7.32 (t, J=8.9 Hz, 2H), 7.17 (d, J=8.2 Hz, 1H), 7.06 (d, J=2.2 Hz, 1H), 6.99 (t, J=7.4 Hz, 1H), 5.90 (d, J=8.0 Hz, 1H), 5.33 (s, 2H), 4.15 (s, 2H), 1.75 (q, J=6.3 Hz, 1H), 0.78-0.49 (m, 4H); MS (ES+): 471.1 (M+1); (ES−): 469.1 (M−1); Analysis calculated for C₂₈H₂₆N₂O₅·1.125HCl·3H₂O: C, 59.46; H, 5.90; Cl, 7.05; N, 4.95; Found: C, 59.62; H, 5.63; Cl, 6.83; N, 4.98; Optical rotation [α]_{D}=+45.714 (c=0.105, MeOH).

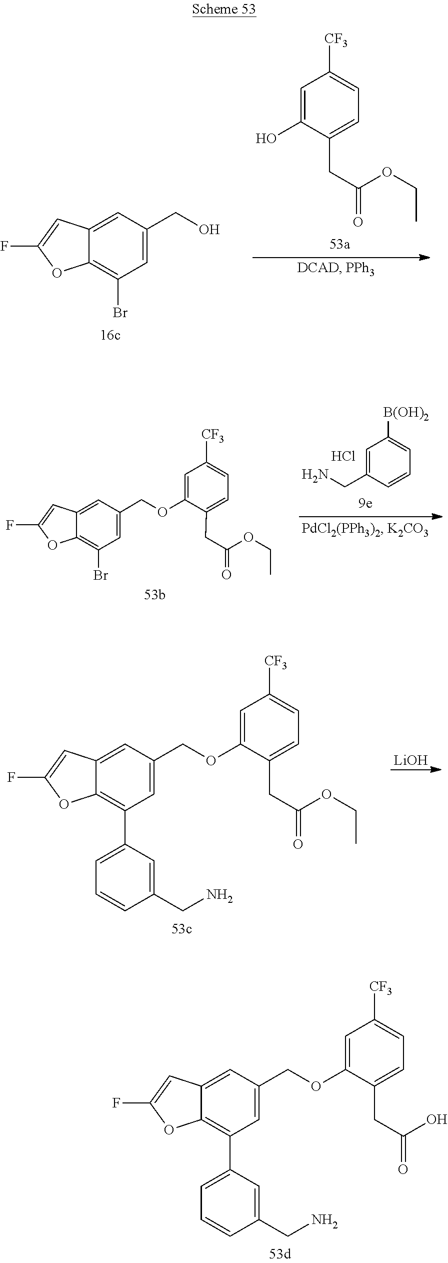

### Preparation of 2-(2-((7-(3-(aminomethyl)phenyl)-2-fluorobenzofuran-5-yl)methoxy)-4-(trifluoromethyl)phenyl)acetic acid (53d)

### Step-1: Preparation of ethyl 2-(2-((7-bromo-2-fluorobenzofuran-5-yl)methoxy)-4-(trifluoromethyl)phenyl)acetate (53b)

Compound 53b was prepared according to the procedure reported in step-2 of scheme 1 from (7-bromo-2-fluorobenzofuran-5-yl)methanol (16c) (1.3 g, 5.31 mmol) in DCM (10 mL) using triphenylphosphine (1.600 g, 6.10 mmol), ethyl 2-(2-hydroxy-4-(trifluoromethyl)phenyl)acetate (53a) (CAS #1261500-50-8, 1.448 g, 5.84 mmol) and (E)-bis(4-chlorobenzyl) diazene-1,2-dicarboxylate (DCAD) (2.240 g, 6.10 mmol) in DCM (30 mL). This gave after workup and purification by flash column chromatography [silica gel (80 g), eluting with EtOAc in hexane from 0-25%]ethyl 2-(2-((7-bromo-2-fluorobenzofuran-5-yl)methoxy)-4-(trifluoromethyl)phenyl)acetate (53b) (2.25 g, 89% yield) as a pale yellow solid; ¹H NMR (300 MHz, DMSO-d₆) δ 7.66 (d, J=1.5 Hz, 1H), 7.61 (d, J=1.5 Hz, 1H), 7.48 (d, J=7.8 Hz, 1H), 7.41 (d, J=1.7 Hz, 1H), 7.32 (d, J=7.8 Hz, 1H), 6.57 (d, J=6.4 Hz, 1H), 5.28 (s, 2H), 4.03 (q, J=7.1 Hz, 2H), 3.73 (s, 2H), 1.08 (t, J=7.1 Hz, 3H); ¹⁹F NMR (282 MHz, DMSO) δ −60.76, −110.33.

### Step-2: Preparation of ethyl 2-(2-((7-(3-(aminomethyl)phenyl)-2-fluorobenzofuran-5-yl)methoxy)-4-(trifluoromethyl)phenyl)acetate (53c)

Compound 53c was prepared according to procedure reported in Step-4 of scheme 1 from ethyl 2-(2-((7-bromo-2-fluorobenzofuran-5-yl)methoxy)-4-(trifluoromethyl)phenyl)acetate (53b) (200 mg, 0.421 mmol) in dioxane (8 mL) using 3-(aminomethyl)phenylboronic acid hydrochloride (9e) (95 mg, 0.505 mmol), bis(triphenylphosphine)Palladium(II)chloride [Pd(PPh₃)₂Cl₂](44.3 mg, 0.063 mmol), a solution of K₂CO₃ (174 mg, 1.263 mmol) in water (1 mL) and heating at 100° C. for 4 h on oil bath. This gave after workup and purification by flash column chromatography [silica gel (24 g), eluting with DMA-80 in DCM from 0-50%]ethyl 2-(2-((7-(3-(aminomethyl)phenyl)-2-fluorobenzofuran-5-yl)methoxy)-4-(trifluoromethyl)phenyl)acetate (53c) (62 mg, 29.4% yield) as a clear oil; ¹H NMR (300 MHz, DMSO-d₆) δ 7.77 (s, 1H), 7.71-7.62 (m, 2H), 7.58 (d, J=1.7 Hz, 1H), 7.53-7.39 (m, 4H), 7.31 (d, J=7.9 Hz, 1H), 6.47 (d, J=6.4 Hz, 1H), 5.33 (s, 2H), 3.93 (q, J=7.1 Hz, 2H), 3.83 (s, 2H), 3.74 (s, 2H), 0.98 (t, J=7.1 Hz, 3H); ¹⁹F NMR (282 MHz, DMSO) δ −60.74, −111.59.

### Step-3: Preparation of 2-(2-((7-(3-(aminomethyl)phenyl)-2-fluorobenzofuran-5-yl)methoxy)-4-(trifluoromethyl)phenyl)acetic acid (53d)

Compound 53d was prepared according to procedure reported in Step-8 of scheme 1 from ethyl 2-(2-((7-(3-(aminomethyl)phenyl)-2-fluorobenzofuran-5-yl)methoxy)-4-(trifluoromethyl)phenyl)acetate (53c) (60 mg, 0.120 mmol) in THE (0.72 mL), acetonitrile (0.36 mL) using a solution of lithium hydroxide monohydrate (1 N, 0.359 mL, 0.359 mmol) and stirring at room temperature for 16 h. This gave after workup and purification by reverse-phase column chromatography [C-18 column, eluting with 0.1% aq HCl in water and acetonitrile from 0-100%]2-(2-((7-(3-(aminomethyl)phenyl)-2-fluorobenzofuran-5-yl)methoxy)-4-(trifluoromethyl)phenyl)acetic acid (53d) (43 mg, 76% yield) HCl salt as a white solid; ¹H NMR (300 MHz, DMSO-d₆) δ 8.34 (s, 3H, D₂O exchangeable), 7.94 (s, 1H), 7.86 (dt, J=7.6, 1.6 Hz, 1H), 7.71 (d, J=1.6 Hz, 1H), 7.66-7.54 (m, 3H), 7.48 (d, J=7.8 Hz, 1H), 7.42 (s, 1H), 7.31 (d, J=7.8 Hz, 1H), 6.48 (d, J=6.4 Hz, 1H), 5.36 (s, 2H), 4.15 (s, 2H), 3.70 (s, 2H); ¹⁹F NMR (282 MHz, DMSO) δ −60.71, −111.43; MS (ES+): 474.0 (M+1); Analysis calculated for C₂₅H₁₉F₄NO₄·HCl·2.25H₂O: C, 54.55; H, 4.49; Cl, 6.44; N, 2.54; Found: C, 54.51; H, 4.11; Cl, 6.56; N, 2.65.

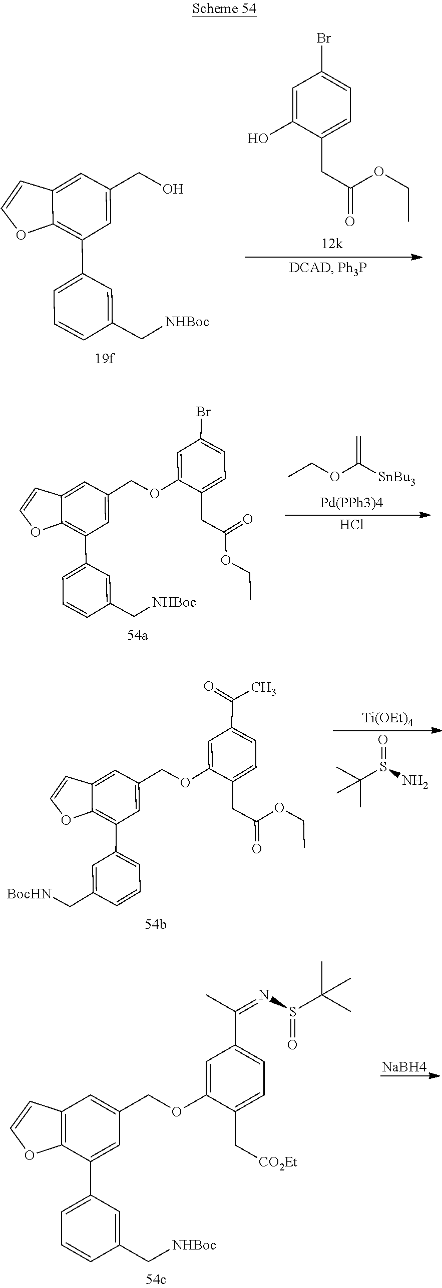

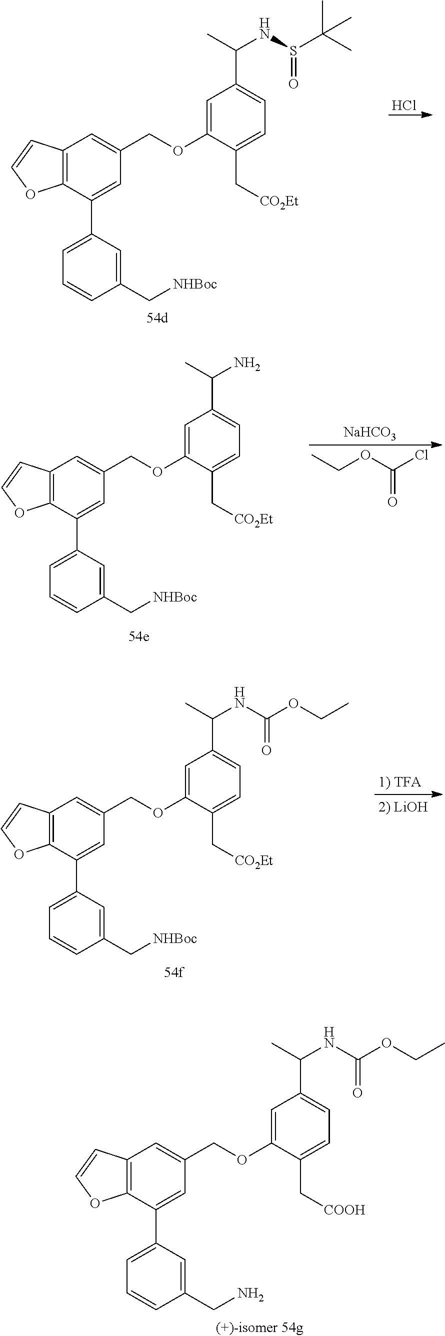

### Preparation of (+)-2-(2-((7-(3-(aminomethyl)phenyl)benzofuran-5-yl)methoxy)-4-(1-(ethoxycarbonylamino)ethyl)phenyl)acetic acid (54g)

### Step-1: Preparation of ethyl 2-(4-bromo-2-((7-(3-(((tert-butoxycarbonyl)amino)methyl)phenyl)benzofuran-5-yl)methoxy)phenyl)acetate (54a)

Compound 54a was prepared according to procedure reported in Step-2 of scheme 1 from tert-butyl 3-(5-(hydroxymethyl)benzofuran-7-yl)benzylcarbamate (19f) (2.68 g, 7.58 mmol) in DCM (20 mL) using triphenylphosphine (3.98 g, 15.17 mmol), ethyl 2-(4-bromo-2-hydroxyphenyl)acetate (12k) (2.014 g, 7.77 mmol) and a solution of (E)-bis(4-chlorobenzyl) diazene-1,2-dicarboxylate (DCAD) (5.57 g, 15.17 mmol) in DCM (10 mL) and stirring at room temperature for 16 h. This gave after workup and purification by flash column chromatography [silica gel, eluting with EtOAc in hexane from 0-20%]ethyl 2-(4-bromo-2-((7-(3-(((tert-butoxycarbonyl)amino)methyl)phenyl)benzofuran-5-yl)methoxy)phenyl)acetate (54a) (3.77 g, 84% yield) as an oil. ¹H NMR (300 MHz, DMSO-d₆) δ 8.10 (d, J=2.2 Hz, 1H), 7.78-7.66 (m, 3H), 7.59-7.44 (m, 3H), 7.34 (d, J=9.0 Hz, 2H), 7.19 (s, 1H), 7.14 (d, J=1.7 Hz, 1H), 7.08 (d, J=2.2 Hz, 1H), 5.27 (s, 2H), 4.23 (d, J=6.2 Hz, 2H), 3.90 (q, J=7.1 Hz, 2H), 3.61 (s, 2H), 1.40 (s, 9H), 0.95 (t, J=7.1 Hz, 3H).

### Step-2: Preparation of ethyl 2-(4-acetyl-2-((7-(3-(((tert-butoxycarbonyl)amino)methyl)phenyl)benzofuran-5-yl)methoxy)phenyl)acetate (54b)

Compound 54b was prepared according to procedure reported in Step-6 of scheme 12 from ethyl 2-(4-bromo-2-((7-(3-(((tert-butoxycarbonyl)amino)methyl)phenyl)benzofuran-5-yl)methoxy)phenyl)acetate (54a) (3.66 g, 6.16 mmol) in toluene (10 mL) using tributyl(1-ethoxyvinyl)stannane (2.74 mL, 7.70 mmol), Pd(PPh₃)₄ (0.711 g, 0.616 mmol) and heating at 100° C. for 16 h. This gave after workup and purification by flash column chromatography [silica gel (24 g), eluting with ethyl acetate in hexanes from 0-25%]ethyl 2-(4-acetyl-2-((7-(3-(((tert-butoxycarbonyl)amino)methyl)phenyl)benzofuran-5-yl)methoxy)phenyl)acetate (54b) (1.9 g, 55.3% yield) as a colorless oil, which solidified upon standing in air. ¹H NMR (300 MHz, DMSO-d₆) δ 8.09 (d, J=2.2 Hz, 1H), 7.80-7.70 (m, 3H), 7.67-7.55 (m, 3H), 7.54-7.44 (m, 3H), 7.39 (t, J=4.1 Hz, 1H), 7.07 (d, J=2.2 Hz, 1H), 5.34 (s, 2H), 4.23 (d, J=6.2 Hz, 2H), 3.92 (q, J=7.1 Hz, 2H), 3.72 (s, 2H), 2.59 (s, 3H), 1.40 (s, 9H), 0.96 (t, J=7.1 Hz, 3H). MS (ES+): 580.1 (M+Na).

### Step-3: Preparation of (R)-ethyl 2-(2-((7-(3-(((tert-butoxycarbonyl)amino)methyl)phenyl)benzofuran-5-yl)methoxy)-4-(1-((tert-butylsulfinyl)imino)ethyl)phenyl)acetate (54c)

Compound 54c was prepared according to procedure reported in Step-5 of scheme 1 from ethyl 2-(4-acetyl-2-((7-(3-(((tert-butoxycarbonyl)amino)methyl)phenyl)benzofuran-5-yl)methoxy)phenyl)acetate (54b) (700 mg, 1.255 mmol) in THE (15 mL) using (R)-2-methylpropane-2-sulfinamide (304 mg, 2.51 mmol), tetraethoxytitanium (859 mg, 3.77 mmol) and heating at 65° C. for 24 h. This gave after workup and purification by flash column chromatography [(silica gel (24 g), eluting with ethyl acetate in hexanes (0 to 30%)](R)-ethyl 2-(2-((7-(3-(((tert-butoxycarbonyl)amino)methyl)phenyl)benzofuran-5-yl)methoxy)-4-(1-((tert-butylsulfinyl)imino)ethyl)phenyl)acetate (54c) (432 mg, 52.1% yield) as a thick yellow oil. ¹H NMR (300 MHz, DMSO-d₆) δ 8.09 (d, J=2.2 Hz, 1H), 7.77-7.66 (m, 3H), 7.55 (d, J=5.3 Hz, 2H), 7.49 (t, J=7.3 Hz, 3H), 7.36 (d, J=7.8 Hz, 1H), 7.30 (d, J=7.5 Hz, 1H), 7.05 (d, J=2.2 Hz, 1H), 5.34 (s, 2H), 4.23 (d, J=6.2 Hz, 2H), 3.96 (q, J=7.1 Hz, 2H), 3.72 (s, 2H), 2.70 (s, 3H), 1.40 (s, 9H), 1.13 (s, 9H), 1.01 (t, J=7.2 Hz, 3H). MS (ES+): 683.1 (M+Na).

### Step-4: Preparation of ethyl 2-(2-((7-(3-(((tert-butoxycarbonyl)amino)methyl)phenyl)benzofuran-5-yl)methoxy)-4-(1-((R)-1,1-dimethylethylsulfinamido)ethyl)phenyl)acetate (54d)

Compound 54d was prepared according to procedure reported in Step-3 of scheme 5 from (R)-ethyl 2-(2-((7-(3-(((tert-butoxycarbonyl)amino)methyl)phenyl)benzofuran-5-yl)methoxy)-4-(1-((tert-butylsulfinyl)imino)ethyl)phenyl)acetate (54c) (420 mg, 0.636 mmol) in THE (10 mL) using sodium borohydride (120 mg, 3.18 mmol). This gave after workup ethyl 2-(2-((7-(3-(((tert-butoxycarbonyl)amino)methyl)phenyl)benzofuran-5-yl)methoxy)-4-(1-((R)-1,1-dimethylethylsulfinamido)ethyl)phenyl)acetate (54d) (410 mg, 97% yield) as a clear colorless oil. ¹H NMR (300 MHz, DMSO-d₆) δ 8.09 (d, J=2.2 Hz, 1H), 7.79-7.70 (m, 3H), 7.56 (d, J=1.6 Hz, 1H), 7.49 (t, J=7.8 Hz, 2H), 7.30 (d, J=7.6 Hz, 1H), 7.21 (d, J=1.5 Hz, 1H), 7.16 (d, J=7.7 Hz, 1H), 7.06 (d, J=2.2 Hz, 1H), 6.98-6.90 (m, 1H), 5.60 (d, J=7.0 Hz, 1H), 5.23 (s, 2H), 4.37 (t, J=6.7 Hz, 1H), 4.23 (d, J=6.2 Hz, 2H), 3.91 (q, J=7.1 Hz, 2H), 3.59 (s, 2H), 1.48-1.31 (m, 12H), 1.10 (s, 9H), 0.96 (t, J=7.1 Hz, 3H). MS (ES+): 685.1 (M+Na).

### Step-5: Preparation of ethyl 2-(4-(1-aminoethyl)-2-((7-(3-(((tert-butoxycarbonyl)amino)methyl)phenyl)benzofuran-5-yl)methoxy)phenyl)acetate (54e)

To a solution of ethyl 2-(2-((7-(3-(((tert-butoxycarbonyl)amino)methyl)phenyl)benzofuran-5-yl)methoxy)-4-(1-((R)-1,1-dimethylethylsulfinamido)ethyl)phenyl)acetate (54d) (405 mg, 0.611 mmol) in THE (10 mL) was added hydrochloric acid (4 M in 1-4-dioxane, 0.306 mL, 1.222 mmol) and stirred for 30 minutes. The reaction was concentrated to dryness to afford ethyl 2-(4-(1-aminoethyl)-2-((7-(3-(((tert-butoxycarbonyl)amino)methyl)phenyl)benzofuran-5-yl)methoxy)phenyl)acetate (54e) (320 mg, 94% yield) as a clear gel. ¹H NMR (300 MHz, DMSO-d₆) δ 8.36 (s, 2H), 8.10 (d, J=2.2 Hz, 1H), 7.80-7.68 (m, 3H), 7.56 (d, J=1.7 Hz, 1H), 7.48 (q, J=7.5 Hz, 2H), 7.38 (s, 1H), 7.36-7.22 (m, 2H), 7.14-6.97 (m, 2H), 5.26 (s, 2H), 4.38 (d, J=7.4 Hz, 1H), 4.23 (d, J=6.2 Hz, 2H), 3.91 (p, J=6.8 Hz, 2H), 3.63 (s, 2H), 1.51 (d, J=6.7 Hz, 3H), 1.38 (d, J=10.8 Hz, 9H), 0.99-0.91 (m, 3H). MS (ES+): 559.1 (M+1).

### Step-6: Preparation of ethyl 2-(2-((7-(3-(((tert-butoxycarbonyl)amino)methyl)phenyl)benzofuran-5-yl)methoxy)-4-(1-((ethoxycarbonyl)amino)ethyl)phenyl)acetate (54f)

Compound 54f was prepared according to procedure reported in Step-6 of scheme 25 from ethyl 2-(4-(1-aminoethyl)-2-((7-(3-(((tert-butoxycarbonyl)amino)methyl)phenyl)benzofuran-5-yl)methoxy)phenyl)acetate (54e) (310 mg, 0.555 mmol) in THF/H₂O (1:1, 6 mL) using NaHCO₃ (140 mg, 1.665 mmol), ethyl carbonochloridate (0.080 mL, 0.832 mmol) and stirring at room temperature for 1 h. The reaction mixture was stirred for 1 h at RT. This gave after workup and purification by flash column chromatography [(silica gel (24 g), eluting with ethyl acetate in hexanes (0 to 50%)]ethyl 2-(2-((7-(3-(((tert-butoxycarbonyl)amino)methyl)phenyl)benzofuran-5-yl)methoxy)-4-(1-((ethoxycarbonyl)amino)ethyl)phenyl)acetate (54f) (240 mg, 68.6% yield) as a colorless oil. ¹H NMR (300 MHz, DMSO-d₆) δ 8.09 (d, J=2.2 Hz, 1H), 7.76 (s, 2H), 7.73 (d, J=1.7 Hz, 2H), 7.66 (d, J=8.4 Hz, 1H), 7.57 (s, 1H), 7.49 (t, J=7.7 Hz, 2H), 7.30 (d, J=7.6 Hz, 1H), 7.14 (d, J=7.0 Hz, 1H), 7.06 (d, J=2.2 Hz, 1H), 6.85 (d, J=7.6 Hz, 1H), 5.21 (s, 2H), 4.66 (t, J=7.5 Hz, 1H), 4.23 (d, J=6.2 Hz, 2H), 4.05-3.86 (m, 4H), 3.57 (s, 2H), 1.40 (s, 9H), 1.33 (d, J=6.9 Hz, 3H), 1.18 (t, J=7.1 Hz, 3H), 0.95 (t, J=7.0 Hz, 3H). MS (ES+): 653.1 (M+Na).

### Step-7: Preparation of (+)-2-(2-((7-(3-(aminomethyl)phenyl)benzofuran-5-yl)methoxy)-4-(1-(ethoxycarbonylamino)ethyl)phenyl)acetic acid (54g)

To a stirred solution of ethyl 2-(2-((7-(3-(((tert-butoxycarbonyl)amino)methyl)phenyl)benzofuran-5-yl)methoxy)-4-(1-((ethoxycarbonyl)amino)ethyl)phenyl)acetate (54f) (120 mg, 0.190 mmol) in DCM (1.5 mL) was added 2,2,2-trifluoroacetic acid (0.147 mL, 1.903 mmol) and for 2 h. The reaction was concentrated in vacuum to dryness. The residue obtained was dissolved in THE (1.5 mL), acetonitrile (0.75 mL), added lithium hydroxide monohydrate, (1N aqueous solution, 0.761 mL, 0.761 mmol) and stirred for 18 h at room temperature. This gave after workup and purification by reverse-phase column chromatography [C-18 column (30 g), eluting with 0.1% aq HCl in water and acetonitrile from 0-100%](+)-2-(2-((7-(3-(aminomethyl)phenyl)benzofuran-5-yl)methoxy)-4-(1-(ethoxycarbonylamino)ethyl)phenyl)acetic acid (54g) (71 mg, 74.3% yield) HCl salt as a white solid; ¹H NMR (300 MHz, DMSO-d₆) δ 12.22 (s, 1H, D₂O exchangeable), 8.47 (s, 3H, D2O exchangeable), 8.12 (d, J=2.2 Hz, 1H), 8.02 (s, 1H), 7.95 (dt, J=7.0, 1.9 Hz, 1H), 7.79 (d, J=1.6 Hz, 1H), 7.73-7.51 (m, 4H), 7.19-7.04 (m, 3H), 6.85 (dd, J=7.6, 1.5 Hz, 1H), 5.25 (s, 2H), 4.64 (p, J=7.3 Hz, 1H), 4.14 (s, 2H), 4.04-3.85 (m, 2H), 3.55 (s, 2H), 1.32 (d, J=7.0 Hz, 3H), 1.14 (t, J=7.1 Hz, 3H); MS (ES+): 503.1 (M+1); (ES−): 501.1 (M−1); Analysis calculated for C₂₉H₃₀N₂O₆·HCl·1.25H₂O: C, 62.03; H, 6.01; Cl, 6.31; N, 4.99; Found: C, 62.13; H, 5.92; Cl, 6.16; N, 4.97. Chiral HPLC: AD-H column 85/15 [(0.1% DEA in n-Heptane in 0.1% DEA in ethanol)]1.0 mL/min UV detection 272 nm, 20 mins run time (Temp 40° C.): 86.20% ee; Optical rotation [α]_{D}=+28.571 (c=0.105 MeOH).

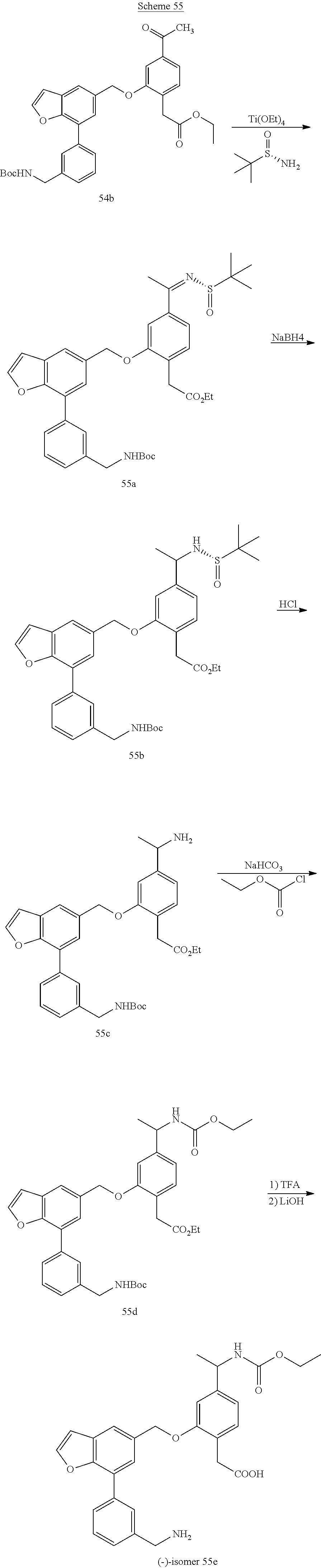

### Preparation of (−)-2-(2-((7-(3-(aminomethyl)phenyl)benzofuran-5-yl)methoxy)-4-(1-((ethoxycarbonyl)amino)ethyl)phenyl)acetic acid (55e)

### Step-1: Preparation of (S)-ethyl 2-(2-((7-(3-(((tert-butoxycarbonyl)amino)methyl)phenyl)benzofuran-5-yl)methoxy)-4-(1-((tert-butylsulfinyl)imino)ethyl)phenyl)acetate (55a)

Compound 55a was prepared according to procedure reported in Step-5 of scheme 1 from ethyl 2-(4-acetyl-2-((7-(3-(((tert-butoxycarbonyl)amino)methyl)phenyl)benzofuran-5-yl)methoxy)phenyl)acetate (54b) (700 mg, 1.255 mmol) in THE (15 mL) using (S)-2-methylpropane-2-sulfinamide (152 mg, 1.255 mmol), tetraethoxytitanium (859 mg, 3.77 mmol) and heating at 65° C. for 24 h. This gave after workup and purification by flash column chromatography [(silica gel (24 g), eluting with ethyl acetate in hexanes (0 to 30%)](S)-ethyl 2-(2-((7-(3-(((tert-butoxycarbonyl)amino)methyl)phenyl)benzofuran-5-yl)methoxy)-4-(1-((tert-butylsulfinyl)imino)ethyl)phenyl)acetate (55a) (396 mg, 47.7% yield) as a thick yellow oil. ¹H NMR (300 MHz, DMSO-d₆) δ 8.09 (d, J=2.2 Hz, 1H), 7.80-7.64 (m, 3H), 7.62-7.53 (m, 2H), 7.47 (d, J=7.3 Hz, 3H), 7.33 (dd, J=17.2, 7.7 Hz, 2H), 7.05 (d, J=2.2 Hz, 1H), 5.34 (s, 2H), 4.23 (d, J=6.2 Hz, 2H), 3.96 (q, J=7.1 Hz, 2H), 3.72 (s, 2H), 2.70 (s, 3H), 1.40 (s, 9H), 1.13 (s, 9H), 1.01 (t, J=7.2 Hz, 3H). MS (ES+): 683.1 (M+Na).

### Step-2: Preparation of ethyl 2-(2-((7-(3-(((tert-butoxycarbonyl)amino)methyl)phenyl)benzofuran-5-yl)methoxy)-4-(1-((S)-1,1-dimethylethylsulfinamido)ethyl)phenyl)acetate (55b)

Compound 55b was prepared according to procedure reported in Step-3 of scheme 5 from (S)-ethyl 2-(2-((7-(3-(((tert-butoxycarbonyl)amino)methyl)phenyl)benzofuran-5-yl)methoxy)-4-(1-((tert-butylsulfinyl)imino)ethyl)phenyl)acetate (55a) (380 mg, 0.575 mmol) in THE (10 mL) using sodium borohydride (109 mg, 2.88 mmol). This gave after workup ethyl 2-(2-((7-(3-(((tert-butoxycarbonyl)amino)methyl)phenyl)benzofuran-5-yl)methoxy)-4-(1-((S)-1,1-dimethylethylsulfinamido)ethyl)phenyl)acetate (55b) (370 mg, 97% yield) as a clear colorless oil. ¹H NMR (300 MHz, DMSO-d₆) δ 8.09 (d, J=2.2 Hz, 1H), 7.80-7.64 (m, 3H), 7.62-7.53 (m, 2H), 7.47 (d, J=7.3 Hz, 3H), 7.33 (dd, J=17.2, 7.7 Hz, 2H), 7.05 (d, J=2.2 Hz, 1H), 5.34 (s, 2H), 4.23 (d, J=6.2 Hz, 2H), 3.96 (q, J=7.1 Hz, 2H), 3.72 (s, 2H), 2.70 (s, 3H), 1.40 (s, 9H), 1.13 (s, 9H), 1.01 (t, J=7.2 Hz, 3H). MS (ES+): 685.1 (M+Na).

### Step-3: Preparation of ethyl 2-(4-(1-aminoethyl)-2-((7-(3-(((tert-butoxycarbonyl)amino)methyl)phenyl)benzofuran-5-yl)methoxy)phenyl)acetate (55c)

To a solution of ethyl 2-(2-((7-(3-(((tert-butoxycarbonyl)amino)methyl)phenyl)benzofuran-5-yl)methoxy)-4-(1-((S)-1,1-dimethylethylsulfinamido)ethyl)phenyl)acetate (55b) (365 mg, 0.551 mmol) in THE (9 mL) was added hydrochloric acid (4 M in 1-4-dioxane, 0.275 mL, 1.101 mmol) and stirred for 30 minutes. The reaction was concentrated to dryness to afford ethyl 2-(4-(1-aminoethyl)-2-((7-(3-(((tert-butoxycarbonyl)amino)methyl)phenyl)benzofuran-5-yl)methoxy)phenyl)acetate (55c) (300 mg, 98% yield) as a clear gel. ¹H NMR (300 MHz, DMSO-d₆) δ 8.26 (s, 2H), 8.11 (d, J=2.3 Hz, 1H), 7.74 (d, J=5.6 Hz, 3H), 7.56 (s, 1H), 7.50 (t, J=7.6 Hz, 2H), 7.38-7.24 (m, 3H), 7.10-6.98 (m, 2H), 5.25 (s, 2H), 4.40 (s, 1H), 4.23 (d, J=6.1 Hz, 2H), 3.90 (q, J=7.0 Hz, 2H), 3.57 (s, 2H), 1.51 (d, J=6.8 Hz, 3H), 1.38 (d, J=10.7 Hz, 9H), 0.95 (t, J=7.0 Hz, 3H). MS (ES+): 559.1 (M+1).

### Step-4: Preparation of ethyl 2-(2-((7-(3-(((tert-butoxycarbonyl)amino)methyl)phenyl)benzofuran-5-yl)methoxy)-4-(1-((ethoxycarbonyl)amino)ethyl)phenyl)acetate (55d)

Compound 55d was prepared according to procedure reported in Step-6 of scheme 25 from ethyl 2-(4-(1-aminoethyl)-2-((7-(3-(((tert-butoxycarbonyl)amino)methyl)phenyl)benzofuran-5-yl)methoxy)phenyl)acetate (55c) (300 mg, 0.537 mmol) in THF/H₂O (1:1, 6 mL) using NaHCO₃ (135 mg, 1.611 mmol), ethyl carbonochloridate (0.077 mL, 0.805 mmol) and stirring at room temperature for 1 h. The reaction mixture was stirred for 1 h at RT. This gave after workup and purification by flash column chromatography [(silica gel (24 g), eluting with ethyl acetate in hexanes (0 to 50%)]ethyl 2-(2-((7-(3-(((tert-butoxycarbonyl)amino)methyl)phenyl)benzofuran-5-yl)methoxy)-4-(1-((ethoxycarbonyl)amino)ethyl)phenyl)acetate (55d) (251 mg, 74.1% yield) as a colorless oil. ¹H NMR (300 MHz, DMSO-d₆) δ 8.09 (d, J=2.2 Hz, 1H), 7.78-7.70 (m, 3H), 7.66 (d, J=8.4 Hz, 1H), 7.57 (s, 1H), 7.49 (t, J=7.8 Hz, 2H), 7.30 (d, J=7.6 Hz, 1H), 7.14 (t, J=3.9 Hz, 2H), 7.06 (d, J=2.3 Hz, 1H), 6.85 (d, J=7.6 Hz, 1H), 5.21 (s, 2H), 4.66 (t, J=7.4 Hz, 1H), 4.23 (d, J=6.2 Hz, 2H), 4.14-3.96 (m, 2H), 3.91-3.84 (m, 2H), 3.57 (s, 2H), 1.40 (s, 9H), 1.33 (d, J=6.9 Hz, 3H), 1.22-1.15 (m, 3H), 0.95 (t, J=7.0 Hz, 3H). MS (ES+): 653.1 (M+Na).

### Step-5: Preparation of (−)-2-(2-((7-(3-(aminomethyl)phenyl)benzofuran-5-yl)methoxy)-4-(1-((ethoxycarbonyl)amino)ethyl)phenyl)acetic acid (55e)

To a stirred solution of ethyl 2-(2-((7-(3-(((tert-butoxycarbonyl)amino)methyl)phenyl)benzofuran-5-yl)methoxy)-4-(1-((ethoxycarbonyl)amino)ethyl)phenyl)acetate (55d) (120 mg, 0.190 mmol) in DCM (1.5 mL) was added 2,2,2-trifluoroacetic acid (0.147 mL, 1.903 mmol) and for 2 h. The reaction was concentrated in vacuum to dryness. The residue obtained was dissolved in THE (1.5 mL), acetonitrile (0.75 mL), added lithium hydroxide monohydrate, (1N aqueous solution, 0.761 mL, 0.761 mmol) and stirred for 18 h at room temperature. This gave after workup and purification by reverse-phase column chromatography [C-18 column (30 g), eluting with 0.1% aq HCl in water and acetonitrile from 0-100%](−)-2-(2-((7-(3-(aminomethyl)phenyl)benzofuran-5-yl)methoxy)-4-(1-((ethoxycarbonyl)amino)ethyl)phenyl)acetic acid (55e) (49 mg, 51.2% yield) HCl salt as a white solid; ¹H NMR (300 MHz, DMSO-d₆) δ 8.47 (s, 2H, D₂O exchangeable), 8.12 (d, J=2.2 Hz, 1H), 8.02 (s, 1H), 7.95 (dt, J=7.1, 1.9 Hz, 1H), 7.79 (d, J=1.6 Hz, 1H), 7.71-7.52 (m, 4H), 7.18-7.03 (m, 3H), 6.85 (d, J=7.6 Hz, 1H), 5.25 (s, 2H), 4.64 (p, J=7.2 Hz, 1H), 4.14 (s, 2H), 4.03-3.82 (m, 2H), 3.55 (s, 2H), 1.32 (d, J=7.0 Hz, 3H), 1.14 (t, J=7.0 Hz, 3H); MS (ES+): 503.1 (M+1); (ES−): 501.1 (M−1); Analysis calculated for C₂₉H₃₀N₂O₆·HCl·1.25H₂O: C, 62.03; H, 6.01; Cl, 6.31; N, 4.99; Found: C, 62.06; H, 5.96; Cl, 6.35; N, 4.97. Chiral HPLC: AD-H column 85/15 [(0.1% DEA in n-Heptane in 0.1% DEA in ethanol)]1.0 mL/min UV detection 272 nm, 20 mins run time (Temp 40° C.) 79.33% ee; Optical rotation [α]_{D}=−28.8 (c=0.125, MeOH).

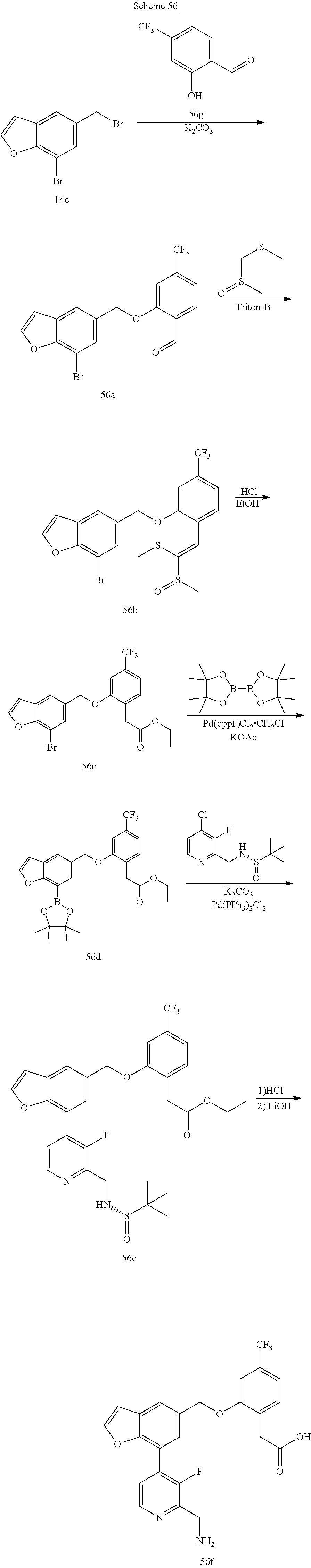

### Preparation of 2-(2-((7-(2-(aminomethyl)-3-fluoropyridin-4-yl)benzofuran-5-yl)methoxy)-4-(trifluoromethyl)phenyl)acetic acid (56f)

### Step-1: Preparation of 2-((7-bromobenzofuran-5-yl)methoxy)-4-(trifluoromethyl)benzaldehyde (56a)

Compound 56a was prepared according to the procedure reported in step-4 of scheme 14 from 7-bromo-5-(bromomethyl)benzofuran (14e) (2.95 g, 10.17 mmol) in DMF (10 mL) using 2-hydroxy-4-(trifluoromethyl)benzaldehyde (56g) (1.934 g, 10.17 mmol), K₂CO₃ (4.22 g, 30.5 mmol) and stirring at room temperature for 16 h. This gave after workup and recrystallization with DCM/Hex 2-((7-bromobenzofuran-5-yl)methoxy)-4-(trifluoromethyl)benzaldehyde (56a) (3.35 g, 82% yield) as a white solid. ¹H NMR (300 MHz, DMSO-d₆) δ 10.45 (s, 1H), 8.17 (d, J=2.2 Hz, 1H), 7.97-7.85 (m, 2H), 7.78 (d, J=1.5 Hz, 1H), 7.72 (s, 1H), 7.47 (d, J=8.1 Hz, 1H), 7.15 (d, J=2.2 Hz, 1H), 5.49 (s, 2H). ¹⁹F NMR (282 MHz, DMSO) δ −61.72.

### Step-2: Preparation of 7-bromo-5-((2-(2-(methylsulfinyl)-2-(methylthio)vinyl)-5-(trifluoromethyl)phenoxy)methyl)benzofuran (56b)

Compound 56b was prepared according to the procedure reported in step-1 of scheme 59 from 2-((7-bromobenzofuran-5-yl)methoxy)-4-(trifluoromethyl)benzaldehyde (56a) (3.3 g, 8.27 mmol) in THE (30 mL) using methyl(methylsulfinylmethyl)sulfane (1.347 mL, 13.23 mmol), Triton-B (40% methanolic solution) (1.879 mL, 4.13 mmol) and heating at reflux for 12 h. This gave after workup and purification by flash column chromatography (SiO₂ (40 g), eluting with 0 to 40% EtOAc in hexane) 7-bromo-5-((2-(2-(methylsulfinyl)-2-(methylthio)vinyl)-5-(trifluoromethyl)phenoxy)methyl)benzofuran (56b) (1.837 g, 44.0% yield) as a white solid. ¹H NMR (300 MHz, DMSO-d₆) δ 8.16 (d, J=2.2 Hz, 1H), 7.83-7.74 (m, 2H), 7.71 (d, J=1.5 Hz, 1H), 7.44 (d, J=1.8 Hz, 1H), 7.34 (d, J=8.2 Hz, 1H), 7.14 (d, J=2.2 Hz, 1H), 6.75 (s, 1H), 5.33 (s, 2H), 2.42 (s, 3H), 2.32 (s, 3H). ¹⁹F NMR (282 MHz, DMSO-d₆) δ −60.77.

### Step-3: Preparation of ethyl 2-(2-((7-bromobenzofuran-5-yl)methoxy)-4-(trifluoromethyl)phenyl)acetate (56c)

Compound 56c was prepared according to the procedure reported in step-2 of scheme 59 from 7-bromo-5-((2-(2-(methylsulfinyl)-2-(methylthio)vinyl)-5-(trifluoromethyl)phenoxy)methyl)benzofuran (56b (1.82 g, 3.60 mmol) in EtOH (45 mL) using hydrochloric acid 4 N in 1,4-dioxane (1.801 mL, 7.20 mmol) and heating at reflux for 15 h. This gave after workup and purification by flash column chromatography (silica gel, 40 g) eluting with EtOAc in hexane from 0-40%) ethyl 2-(2-((7-bromobenzofuran-5-yl)methoxy)-4-(trifluoromethyl)phenyl)acetate (56c) (1.47 g, 89% yield) as a white solid. ¹H NMR (300 MHz, DMSO-d₆) δ 8.17 (d, J=2.2 Hz, 1H), 7.74 (d, J=1.5 Hz, 1H), 7.62 (d, J=1.5 Hz, 1H), 7.48 (d, J=7.8 Hz, 1H), 7.42 (s, 1H), 7.32 (d, J=7.8 Hz, 1H), 7.14 (d, J=2.2 Hz, 1H), 5.29 (s, 2H), 4.02 (q, J=7.1 Hz, 2H), 3.73 (s, 2H), 1.06 (t, J=7.1 Hz, 3H). ¹⁹F NMR (282 MHz, DMSO) δ −60.76.

### Step-4: Preparation of ethyl 2-(2-((7-(4,4,5,5-tetramethyl-1,3,2-dioxaborolan-2-yl)benzofuran-5-yl)methoxy)-4-(trifluoromethyl)phenyl)acetate (56d)

Compound 56d was prepared according to the procedure reported in step-3 of scheme 1 from ethyl 2-(2-((7-bromobenzofuran-5-yl)methoxy)-4-(trifluoromethyl)phenyl)acetate (56c) (1 g, 2.187 mmol) using bis(pinacolato)diboron (0.833 g, 3.28 mmol), potassium acetate (0.644 g, 6.56 mmol) and PdCl₂(dppf)-CH₂Cl₂ (0.179 g, 0.219 mmol) in anhydrous dioxane (25 mL) under an nitrogen atmosphere and heating at 90° C. overnight. This gave after workup and purification by flash column chromatography [silica (40 g), eluting with EtOAc in hexane from 0-40%]ethyl 2-(2-((7-(4,4,5,5-tetramethyl-1,3,2-dioxaborolan-2-yl)benzofuran-5-yl)methoxy)-4-(trifluoromethyl)phenyl)acetate (56d) (960 mg, 87% yield) as a clear oil. ¹H NMR (300 MHz, DMSO-d₆) δ 8.08 (d, J=2.2 Hz, 1H), 7.85 (d, J=1.8 Hz, 1H), 7.68 (d, J=1.8 Hz, 1H), 7.51-7.42 (m, 2H), 7.30 (d, J=7.8 Hz, 1H), 6.99 (d, J=2.2 Hz, 1H), 5.29 (s, 2H), 4.01 (q, J=7.1 Hz, 2H), 3.69 (s, 2H), 1.34 (s, 12H), 1.04 (t, J=7.1 Hz, 3H). ¹⁹F NMR (282 MHz, DMSO) δ −60.76.

### Step-5: Preparation of ethyl 2-(2-((7-(2-((1,1-dimethylethylsulfinamido)methyl)-3-fluoropyridin-4-yl)benzofuran-5-yl)methoxy)-4-(trifluoromethyl)phenyl)acetate (56e)

Compound 56e was prepared according to procedure reported in Step-4 of scheme 1 from ethyl 2-(2-((7-(4,4,5,5-tetramethyl-1,3,2-dioxaborolan-2-yl)benzofuran-5-yl)methoxy)-4-(trifluoromethyl)phenyl)acetate (56d) (240 mg, 0.476 mmol) in dioxane (10 mL) using (+)-N-((4-chloro-3-fluoropyridin-2-yl)methyl)-2-methylpropane-2-sulfinamide (11c) (126 mg, 0.476 mmol), bis(triphenylphosphine)Palladium(II)chloride [Pd(PPh₃)₂Cl₂](50.1 mg, 0.071 mmol), a solution of K₂CO₃ (197 mg, 1.428 mmol) in water (1 mL) and heating at 100° C. for 5 h on oil bath. This gave after workup and purification by flash column chromatography [silica gel (24 g), eluting with DMA-80 in DCM from 0-30%]ethyl 2-(2-((7-(2-((1,1-dimethylethylsulfinamido)methyl)-3-fluoropyridin-4-yl)benzofuran-5-yl)methoxy)-4-(trifluoromethyl)phenyl)acetate (56e) (261 mg, 90% yield) as a brown oil. ¹H NMR (300 MHz, DMSO-d₆) δ 8.53 (d, J=5.0 Hz, 1H), 8.11 (d, J=2.2 Hz, 1H), 7.88 (d, J=1.6 Hz, 1H), 7.71-7.65 (m, 2H), 7.56 (t, J=2.1 Hz, 1H), 7.46 (s, 1H), 7.36-7.25 (m, 1H), 7.12 (d, J=2.2 Hz, 1H), 5.87 (t, J=5.6 Hz, 1H), 5.36 (s, 2H), 4.42 (d, J=5.8 Hz, 2H), 3.91 (q, J=7.2 Hz, 2H), 3.57 (s, 2H), 1.12 (s, 9H), 0.96 (t, J=7.1 Hz, 3H). ¹⁹F NMR (282 MHz, DMSO) δ −60.75, −128.01. MS (ES+): 607.1 (M+1).

### Step-6: Preparation of 2-(2-((7-(2-(aminomethyl)-3-fluoropyridin-4-yl)benzofuran-5-yl)methoxy)-4-(trifluoromethyl)phenyl)acetic acid (56f)

To a solution of ethyl 2-(2-((7-(2-((1,1-dimethylethylsulfinamido)methyl)-3-fluoropyridin-4-yl)benzofuran-5-yl)methoxy)-4-(trifluoromethyl)phenyl)acetate (56e) (255 mg, 0.420 mmol) in THE (6 mL) was added hydrochloric acid (4M in 1-4-dioxane, 0.210 mL, 0.841 mmol) and stirred for 30 mins. The reaction was concentrated in vacuum to dryness. The residue obtained was dissolved in THE (4 mL), acetonitrile (2 mL), added lithium hydroxide monohydrate, 1N (2.102 mL, 2.102 mmol) and stirred for 18 h at room temperature. This gave after workup and purification by reverse-phase column chromatography [C-18 column (50 g), eluting with 0.1% aq HCl in water and acetonitrile from 0-100%]2-(2-((7-(2-(aminomethyl)-3-fluoropyridin-4-yl)benzofuran-5-yl)methoxy)-4-(trifluoromethyl)phenyl)acetic acid (56f) (96 mg, 48.1% yield) HCl salt as a white solid; ¹H NMR (300 MHz, DMSO-d₆) δ 12.40 (s, 1H, D₂O exchangeable), 8.65 (d, J=5.0 Hz, 1H), 8.58 (d, J=5.9 Hz, 3H, D₂O exchangeable), 8.14 (d, J=2.2 Hz, 1H), 7.94 (d, J=1.6 Hz, 1H), 7.81 (t, J=5.3 Hz, 1H), 7.63 (s, 1H), 7.54-7.39 (m, 2H), 7.31 (dd, J=7.8, 1.6 Hz, 1H), 7.13 (d, J=2.2 Hz, 1H), 5.40 (s, 2H), 4.39 (d, J=5.8 Hz, 2H), 3.69 (s, 2H); ¹⁹F NMR (282 MHz, DMSO) δ −60.73, −128.41; MS (ES+): 475.0 (M+1), (ES−): 947.1 (2M−1); Analysis calculated for C₂₄H₁₈F₄N₂O₄·HCl·0.5H₂O: C, 55.45; H, 3.88; Cl, 6.82; N, 5.39; Found: C, 55.27; H, 3.76; Cl, 7.06; N, 5.39.

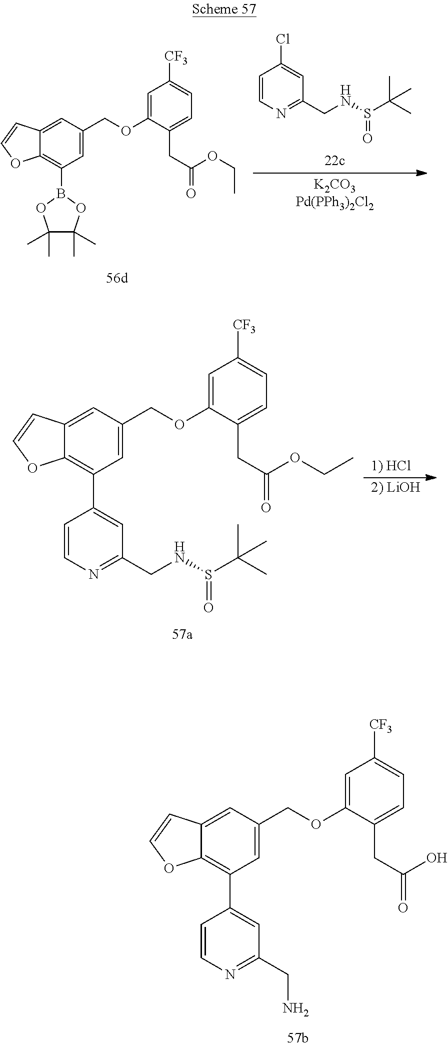

### Preparation of 2-(2-((7-(2-(aminomethyl)pyridin-4-yl)benzofuran-5-yl)methoxy)-4-(trifluoromethyl)phenyl)acetic acid (57b)

### Step-1: Preparation of ethyl 2-(2-((7-(2-((1,1-dimethylethylsulfinamido)methyl)pyridin-4-yl)benzofuran-5-yl)methoxy)-4-(trifluoromethyl)phenyl)acetate (57a)

Compound 57a was prepared according to procedure reported in Step-4 of scheme 1 from ethyl 2-(2-((7-(4,4,5,5-tetramethyl-1,3,2-dioxaborolan-2-yl)benzofuran-5-yl)methoxy)-4-(trifluoromethyl)phenyl)acetate (56d) (240 mg, 0.476 mmol) in dioxane (10 mL) using (+)-N-((4-chloropyridin-2-yl)methyl)-2-methylpropane-2-sulfinamide (22c) (117 mg, 0.476 mmol), bis(triphenylphosphine)Palladium(II)chloride [Pd(PPh₃)₂Cl₂](50.1 mg, 0.071 mmol), a solution of K₂CO₃ (197 mg, 1.428 mmol) in water (1 mL) and heating at 100° C. for 5 h on oil bath. This gave after workup and purification by flash column chromatography [silica gel (24 g), eluting with DMA-80 in DCM from 0-30%]ethyl 2-(2-((7-(2-((1,1-dimethylethylsulfinamido)methyl)pyridin-4-yl)benzofuran-5-yl)methoxy)-4-(trifluoromethyl)phenyl)acetate (57a) (232 mg, 83% yield) as a brown oil. ¹H NMR (300 MHz, DMSO-d₆) δ 8.69-8.62 (m, 1H), 8.14 (d, J=2.2 Hz, 1H), 8.07 (d, J=1.6 Hz, 1H), 7.84 (d, J=1.5 Hz, 1H), 7.79 (dd, J=5.3, 1.7 Hz, 1H), 7.75 (d, J=1.6 Hz, 1H), 7.46 (d, J=2.7 Hz, 2H), 7.32 (d, J=7.8 Hz, 1H), 7.13 (d, J=2.2 Hz, 1H), 5.99-5.98 (m, 1H), 5.36 (s, 2H), 4.38 (t, J=5.4 Hz, 2H), 3.92-3.85 (m, 2H), 3.74 (s, 2H), 1.18 (s, 9H), 0.94 (t, J=7.1 Hz, 3H). ¹⁹F NMR (282 MHz, DMSO) δ −60.74. MS (ES+): 589.1 (M+1).

### Step-2: Preparation of 2-(2-((7-(2-(aminomethyl)pyridin-4-yl)benzofuran-5-yl)methoxy)-4-(trifluoromethyl)phenyl)acetic acid (57b)

Compound 57b was prepared according to procedure reported in Step-6 of scheme 56 from ethyl 2-(2-((7-(2-((1,1-dimethylethylsulfinamido)methyl)pyridin-4-yl)benzofuran-5-yl)methoxy)-4-(trifluoromethyl)phenyl)acetate (57a) (220 mg, 0.374 mmol) using hydrochloric acid (4.M in 1-4-dioxane) (0.187 mL, 0.747 mmol) for hydrolysis of sulfinamido protecting group and lithium hydroxide monohydrate, 1N (1.869 mL, 1.869 mmol) for hydrolysis of ester. This gave after workup and purification by reverse-phase column chromatography [C-18 column (50 g), eluting with 0.1% aq HCl in water and acetonitrile from 0-100%]2-(2-((7-(2-(aminomethyl)pyridin-4-yl)benzofuran-5-yl)methoxy)-4-(trifluoromethyl)phenyl)acetic acid (57b) (79 mg, 46.3% yield) HCl salt as a white solid; ¹H NMR (300 MHz, DMSO-d₆) δ 8.79 (d, J=5.3 Hz, 1H), 8.49 (s, 3H, D2O exchangeable), 8.19 (d, J=2.2 Hz, 1H), 8.08 (d, J=1.7 Hz, 1H), 7.99 (dd, J=5.3, 1.7 Hz, 1H), 7.90 (d, J=1.6 Hz, 1H), 7.85 (d, J=1.6 Hz, 1H), 7.52-7.41 (m, 2H), 7.31 (d, J=7.8 Hz, 1H), 7.14 (d, J=2.2 Hz, 1H), 5.40 (s, 2H), 4.32 (q, J=5.6 Hz, 2H), 3.71 (s, 2H); ¹⁹F NMR (282 MHz, DMSO) δ −60.73; MS (ES+): 457.0 (M+1); Analysis calculated for C₂₄H₁₉F₃N₂O₄·1.6HCl·1.25H₂O: C, 53.65; H, 4.33; Cl, 10.56; N, 5.21; Found: C, 53.40; H, 4.09; Cl, 10.58; N, 5.19.

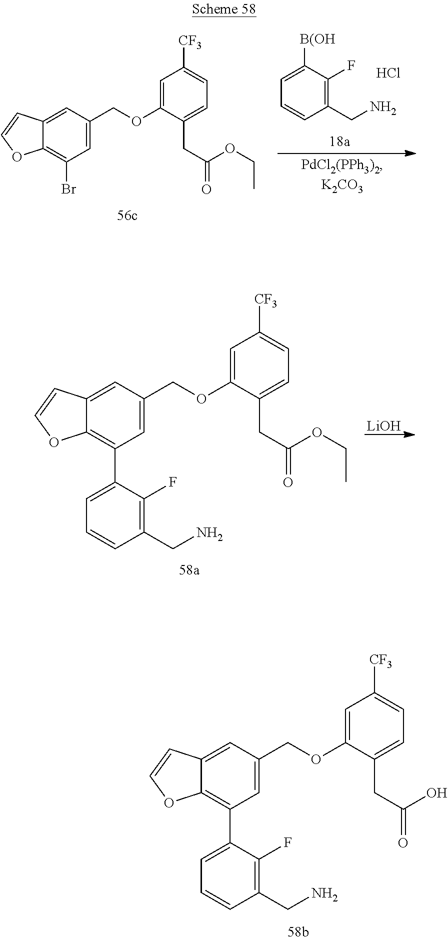

### Preparation of 2-(2-((7-(3-(aminomethyl)-2-fluorophenyl)benzofuran-5-yl)methoxy)-4-(trifluoromethyl)phenyl)acetic acid (58b)

### Step-1: Preparation of ethyl 2-(2-((7-(3-(aminomethyl)-2-fluorophenyl)benzofuran-5-yl)methoxy)-4-(trifluoromethyl)phenyl)acetate (58a)

Compound 58a was prepared according to procedure reported in Step-4 of scheme 1 from ethyl 2-(2-((7-bromobenzofuran-5-yl)methoxy)-4-(trifluoromethyl)phenyl)acetate (56c) (235 mg, 0.514 mmol) in dioxane (20 mL) using (3-(aminomethyl)-2-fluorophenyl)boronic acid hydrochloride (18a) (132 mg, 0.642 mmol), bis(triphenylphosphine)Palladium(II)chloride [Pd(PPh₃)₂Cl₂](54.1 mg, 0.077 mmol), a solution of K₂CO₃ (213 mg, 1.542 mmol) in water (4 mL) and heating at 100° C. for 4 h on oil bath. This gave after workup and purification by flash column chromatography [silica gel (24 g), eluting with 0 to 50% DMA-80 in DCM]ethyl 2-(2-((7-(3-(aminomethyl)-2-fluorophenyl)benzofuran-5-yl)methoxy)-4-(trifluoromethyl)phenyl)acetate (58a) (114 mg, 44.2% yield) as a clear oil. ¹H NMR (300 MHz, DMSO-d₆) δ 8.06 (d, J=2.2 Hz, 1H), 7.77 (d, J=1.6 Hz, 1H), 7.65-7.55 (m, 1H), 7.46 (t, J=7.4 Hz, 4H), 7.32 (t, J=7.8 Hz, 2H), 7.07 (d, J=2.2 Hz, 1H), 5.34 (s, 2H), 3.91 (q, J=7.1 Hz, 2H), 3.84 (s, 2H), 3.72 (s, 2H), 0.97 (t, J=7.1 Hz, 3H). ¹⁹F NMR (282 MHz, DMSO) δ −60.74, −121.78; MS (ES+): 502.0 (M+1).

### Step-2: Preparation of 2-(2-((7-(3-(aminomethyl)-2-fluorophenyl)benzofuran-5-yl)methoxy)-4-(trifluoromethyl)phenyl)acetic acid (58b)

Compound 58b was prepared according to procedure reported in Step-8 of scheme 1 from ethyl 2-(2-((7-(3-(aminomethyl)-2-fluorophenyl)benzofuran-5-yl)methoxy)-4-(trifluoromethyl)phenyl)acetate (58a) (110 mg, 0.219 mmol) in THE (1.3 mL), acetonitrile (0.65 mL) using a 1 N solution of lithium hydroxide monohydrate (0.658 mL, 0.658 mmol) and stirring for 16 h at room temperature. This gave after workup and purification by reverse-phase column chromatography [C-18 column (50 g), eluting with 0.1% aq HCl in water and acetonitrile from 0-100%]2-(2-((7-(3-(aminomethyl)-2-fluorophenyl)benzofuran-5-yl)methoxy)-4-(trifluoromethyl)phenyl)acetic acid (58b) (87 mg, 84% yield) HCl salt as a white solid; ¹H NMR (300 MHz, DMSO-d₆) δ 8.07 (d, J=2.2 Hz, 1H), 7.85 (d, J=1.6 Hz, 1H), 7.69 (tt, J=8.8, 1.8 Hz, 2H), 7.58-7.34 (m, 4H), 7.34-7.22 (m, 1H), 7.08 (d, J=2.2 Hz, 1H), 5.37 (s, 2H), 4.19 (s, 2H), 3.68 (s, 2H); ¹⁹F NMR (282 MHz, DMSO) δ −60.73, −118.39; MS (ES+): 474.0 (M+1); Analysis calculated for C₂₅H₁₉F₄NO₄·HCl·H₂O: C, 56.88; H, 4.20; Cl, 6.72; N, 2.65; Found: C, 57.14; H, 3.93; Cl, 6.96; N, 2.78.

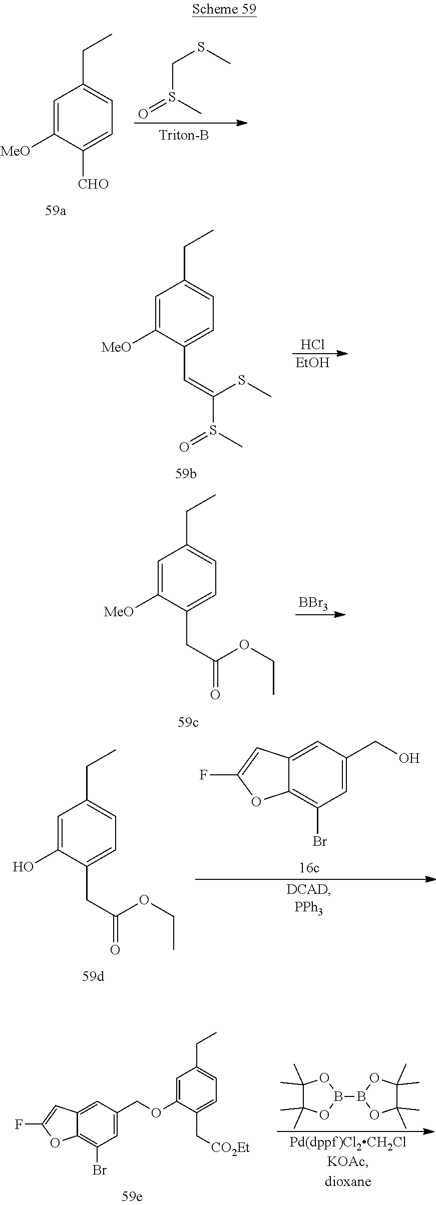

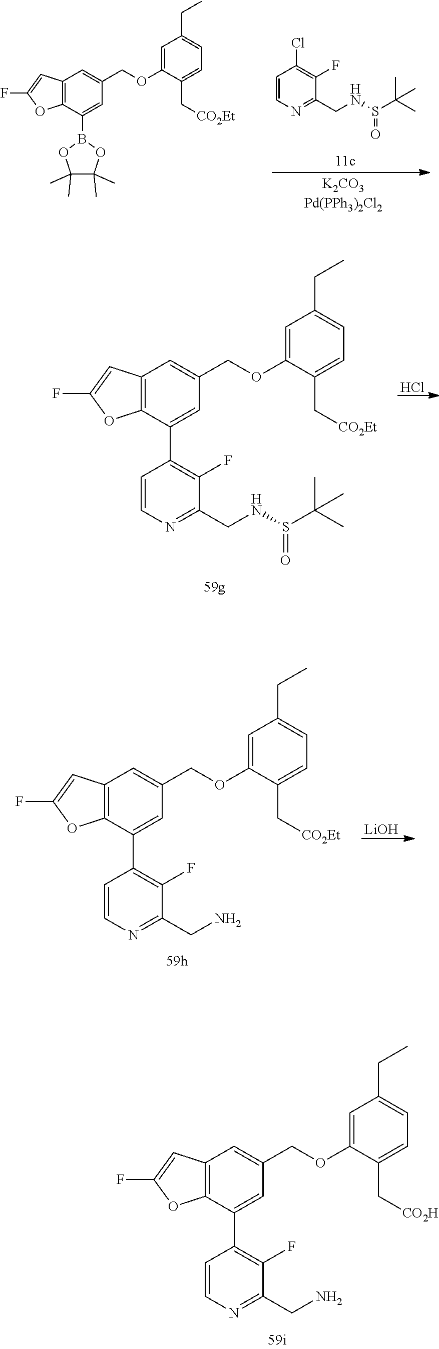

### Preparation of 2-(2-((7-(2-(aminomethyl)-3-fluoropyridin-4-yl)-2-fluorobenzofuran-5-yl)methoxy)-4-ethylphenyl)acetic acid (59i)

### Step-1: Preparation of (2-(4-ethyl-2-methoxyphenyl)-1-(methylsulfinyl)vinyl)(methyl)sulfane (59b)

To a solution of from 4-ethyl-2-methoxybenzaldehyde (59a) (1.82 g, 11.08 mmol; CAS #142224-35-9) in THE (20 mL) was added at room temperature methyl(methylsulfinylmethyl)sulfane (2.203 g, 17.73 mmol), Triton-B (40% methanolic solution; 2.317 g, 5.54 mmol) and heated at 70° C. for 16 h. The resulting black solution was cooled to room temperature diluted with H₂O (30 mL) and EtOAc (30 mL). After 30-min stirring, the two layers were separated. The aqueous layer was extracted with EtOAc (2×25 mL). The combined organic extract was washed with H₂O (30 mL), brine (30 mL), dried, filtered and concentrated in vacuum. The residue obtained was purified by flash column chromatography (SiO₂ (24 g), eluting with 0-40% EtOAc in hexane) to afford (2-(4-ethyl-2-methoxyphenyl)-1-(methylsulfinyl)vinyl)(methyl)sulfane (59b) (2.66 g, 89% yield) as a pale-yellow oil; ¹H NMR (300 MHz, DMSO-d₆) δ 8.01 (d, J=7.9 Hz, 1H), 7.75 (s, 1H), 6.95 (d, J=1.6 Hz, 1H), 6.93-6.83 (m, 1H), 3.84 (s, 3H), 2.71 (s, 3H), 2.64 (q, J=7.6 Hz, 2H), 2.28 (s, 3H), 1.22 (t, J=7.6 Hz, 3H); MS (ES+): 271 (M+1).

### Step-2: Preparation of ethyl 2-(4-ethyl-2-methoxyphenyl)acetate (59c)

To a solution of (2-(4-ethyl-2-methoxyphenyl)-1-(methylsulfinyl)vinyl)(methyl)sulfane (59b) (2.66 g, 9.84 mmol) in EtOH (20 mL) was added 4 M HCl in dioxane (12.30 mL, 49.2 mmol) and heated at 80° C. for 16 h. The cooled yellow solution was evaporated to remove EtOH. The concentrate was diluted with saturated NaHCO₃ (20 mL) and EtOAc (20 mL). After 30-min stirring, the two layers were separated. The aqueous layer was extracted with EtOAc (2×20 mL). The combined organic extract was washed with H₂O (30 mL), brine (30 mL), dried, filtered and concentrated in vacuum. The residue obtained was purified by flash column chromatography (SiO₂ (24 g), eluting with 0-2% EtOAc in hexane) to afford ethyl 2-(4-ethyl-2-methoxyphenyl)acetate (59c) (1.61 g, 74% yield) as a colorless liquid; ¹H NMR (300 MHz, DMSO-d₆) δ 7.06 (d, J=7.5 Hz, 1H), 6.82 (d, J=1.6 Hz, 1H), 6.73 (dd, J=7.5, 1.6 Hz, 1H), 4.05 (q, J=7.1 Hz, 2H), 3.74 (s, 3H), 3.51 (s, 2H), 2.58 (q, J=7.6 Hz, 2H), 1.22-1.13 (m, 6H); MS (ES+): 245 (M+Na).

### Step-3: Preparation of ethyl 2-(4-ethyl-2-hydroxyphenyl)acetate (59d)

Compound 59d was prepared according to the procedure reported in step-2 of scheme 19 from ethyl 2-(4-ethyl-2-methoxyphenyl)acetate (59c) (1.61 g, 7.24 mmol) in dichloromethane (15 mL) using boron tribromide (14.49 mL, 14.49 mmol; 1M solution in DCM) and stirring at 0° C. for 2 h. This gave after workup purification by flash column chromatography (silica gel, eluting with 0-10% EtOAc in hexane) to provide the product ethyl 2-(4-ethyl-2-hydroxyphenyl)acetate (59d) (472 mg, 2.266 mmol, 31% yield) as a yellow oil; ¹H NMR (300 MHz, DMSO-d₆) δ 9.32 (s, 1H), 6.98 (d, J=7.6 Hz, 1H), 6.62 (d, J=1.7 Hz, 1H), 6.58 (dd, J=7.6, 1.7 Hz, 1H), 4.05 (q, J=7.1 Hz, 2H), 3.48 (s, 2H), 2.49-2.43 (m, 2H), 1.15 (dt, J=10.4, 7.3 Hz, 6H); MS (ES+): 209 (M+1).

### Step-4: Preparation of ethyl 2-(2-((7-bromo-2-fluorobenzofuran-5-yl)methoxy)-4-ethylphenyl)acetate (59e)

Compound 59e was prepared according to the procedure reported in step-2 of scheme 1 from (7-bromo-2-fluorobenzofuran-5-yl)methanol (16c) (555 mg, 2.266 mmol) in DCM (20 mL) using triphenylphosphine (892 mg, 3.40 mmol), ethyl 2-(4-ethyl-2-hydroxyphenyl)acetate (59d) (472 mg, 2.266 mmol) and a solution of (E)-bis(4-chlorobenzyl) diazene-1,2-dicarboxylate (DCAD) (1248 mg, 3.40 mmol) in DCM (20 mL). This gave after workup and purification by flash column chromatography (silica gel (24 g), eluting with 0 to 5% ethyl acetate in hexanes) ethyl 2-(2-((7-bromo-2-fluorobenzofuran-5-yl)methoxy)-4-ethylphenyl)acetate (59e) (575 mg, 58% yield) as a pale-yellow oil. ¹H NMR (300 MHz, DMSO-d₆) δ 7.64 (d, J=1.5 Hz, 1H), 7.60 (d, J=1.5 Hz, 1H), 7.11 (d, J=7.5 Hz, 1H), 6.94 (d, J=1.6 Hz, 1H), 6.76 (dd, J=7.6, 1.5 Hz, 1H), 6.54 (d, J=6.4 Hz, 1H), 5.16 (s, 2H), 4.01 (q, J=7.1 Hz, 2H), 3.57 (s, 2H), 2.58 (q, J=7.7 Hz, 2H), 1.18 (t, J=7.6 Hz, 3H), 1.08 (t, J=7.1 Hz, 3H). ¹⁹F NMR (282 MHz, DMSO-d₆) δ −110.50; MS (ES+): 435/437 (M+1).

### Step-5: Preparation of ethyl 2-(4-ethyl-2-((2-fluoro-7-(4,4,5,5-tetramethyl-1,3,2-dioxaborolan-2-yl)benzofuran-5-yl)methoxy)phenyl)acetate (59f)

Compound 59f was prepared according to the procedure reported in step-3 of scheme 1 from ethyl 2-(2-((7-bromo-2-fluorobenzofuran-5-yl)methoxy)-4-ethylphenyl)acetate (59e) (350 mg, 0.804 mmol) using bis(pinacolato)diboron (306 mg, 1.206 mmol), potassium acetate (237 mg, 2.412 mmol) and PdCl₂(dppf)-CH₂Cl₂ (66 mg, 0.080 mmol) in anhydrous dioxane (5 mL) under an nitrogen atmosphere and heating at 100° C. for 16 h. This gave after workup and purification by flash column chromatography [silica (24 g), eluting with EtOAc in hexane from 0-5%]ethyl 2-(4-ethyl-2-((2-fluoro-7-(4,4,5,5-tetramethyl-1,3,2-dioxaborolan-2-yl)benzofuran-5-yl)methoxy)phenyl)acetate (59f) (326 mg, 84% yield) as an opaque oil; ¹H NMR (300 MHz, DMSO-d₆) δ 7.75 (s, 1H), 7.63 (s, 1H), 7.09 (d, J=7.5 Hz, 1H), 6.97 (s, 1H), 6.75 (d, J=7.6 Hz, 1H), 6.35 (d, J=6.4 Hz, 1H), 5.15 (s, 2H), 3.99 (q, J=7.1 Hz, 2H), 3.53 (s, 2H), 2.58 (q, J=7.7 Hz, 2H), 1.19-1.13 (m, 15H), 1.05 (t, J=7.1 Hz, 3H); ¹⁹F NMR (282 MHz, DMSO-d₆) δ −111.75; MS (ES+): 505.2 (M+Na).

### Step-6: Preparation of ethyl 2-(2-((7-(2-((1,1-dimethylethylsulfinamido)methyl)-3-fluoropyridin-4-yl)-2-fluorobenzofuran-5-yl)methoxy)-4-ethylphenyl)acetate (59g)

Compound 59g was prepared according to procedure reported in Step-4 of scheme 1 from ethyl 2-(4-ethyl-2-((2-fluoro-7-(4,4,5,5-tetramethyl-1,3,2-dioxaborolan-2-yl)benzofuran-5-yl)methoxy)phenyl)acetate (59f) (185 mg, 0.384 mmol) in dioxane (4 mL) using (+)-N-((4-chloro-3-fluoropyridin-2-yl)methyl)-2-methylpropane-2-sulfinamide (11c) (112 mg, 0.422 mmol), bis(triphenylphosphine)Palladium(II)chloride [Pd(PPh₃)₂Cl₂](26.9 mg, 0.038 mmol), a 3.3 M aqueous K₂CO₃ (0.349 mL, 1.151 mmol) and heating at 100° C. for 16 h on oil bath. This gave after workup and purification by flash column chromatography [silica gel (40 g), eluting with 0 to 3% methanol in DCM]ethyl 2-(2-((7-(2-((1,1-dimethylethylsulfinamido)methyl)-3-fluoropyridin-4-yl)-2-fluorobenzofuran-5-yl)methoxy)-4-ethylphenyl)acetate (59g) (173 mg, 77% yield) as an orange oil; MS (ES+): 585 (M+1).

### Step-7: Preparation of ethyl 2-(2-((7-(2-(aminomethyl)-3-fluoropyridin-4-yl)-2-fluorobenzofuran-5-yl)methoxy)-4-ethylphenyl)acetate (59h)

To a solution of ethyl 2-(2-((7-(2-((1,1-dimethylethylsulfinamido)methyl)-3-fluoropyridin-4-yl)-2-fluorobenzofuran-5-yl)methoxy)-4-ethylphenyl)acetate (59g) (173 mg, 0.296 mmol) in tetrahydrofuran (4 mL) was added 4 M HCl in dioxane (0.222 mL, 0.888 mmol) and stirred at RT for 16 h. The reaction mixture was concentrated to dryness to afford ethyl 2-(2-((7-(2-(aminomethyl)-3-fluoropyridin-4-yl)-2-fluorobenzofuran-5-yl)methoxy)-4-ethylphenyl)acetate (59h) which was used as such in the next step without further purification; MS (ES+): 481 (M+1).

### Step-8: Preparation of 2-(2-((7-(2-(aminomethyl)-3-fluoropyridin-4-yl)-2-fluorobenzofuran-5-yl)methoxy)-4-ethylphenyl)acetic acid (59i)

Compound 59i was prepared according to procedure reported in Step-8 of scheme 1 from ethyl 2-(2-((7-(2-(aminomethyl)-3-fluoropyridin-4-yl)-2-fluorobenzofuran-5-yl)methoxy)-4-ethylphenyl)acetate (59h) (102 mg, 0.212 mmol) in THE (4 mL) using 2.0 M aqueous LiOH (1.061 mL, 2.123 mmol) and heating at 40° C. 16 h. This gave after workup and purification by reverse-phase column chromatography [C-18 column, eluting with 0.1% aq HCl in water and acetonitrile from 0-100%]2-(2-((7-(2-(aminomethyl)-3-fluoropyridin-4-yl)-2-fluorobenzofuran-5-yl)methoxy)-4-ethylphenyl)acetic acid (59i) (14 mg, 15% yield) HCl salt as a pale-yellow solid; ¹H NMR (300 MHz, DMSO-d₆) δ 12.15 (s, 1H, D₂O exchangeable), 8.64 (d, J=4.9 Hz, 1H), 8.54 (s, 3H, D₂O exchangeable), 7.84 (s, 1H), 7.80 (t, J=5.3 Hz, 1H), 7.60 (s, 1H), 7.11 (d, J=7.6 Hz, 1H), 6.97 (s, 1H), 6.76 (d, 1H), 6.51 (d, J=6.4 Hz, 1H), 5.25 (s, 2H), 4.39 (d, J=5.7 Hz, 2H), 3.53 (s, 2H), 2.58 (q, J=7.6 Hz, 2H), 1.18 (t, J=7.5 Hz, 3H); ¹⁹F NMR (282 MHz, DMSO-d₆) δ −111.22, −128.55; MS (ES+): 453 (M+1), (ES−): 451 (M−1).

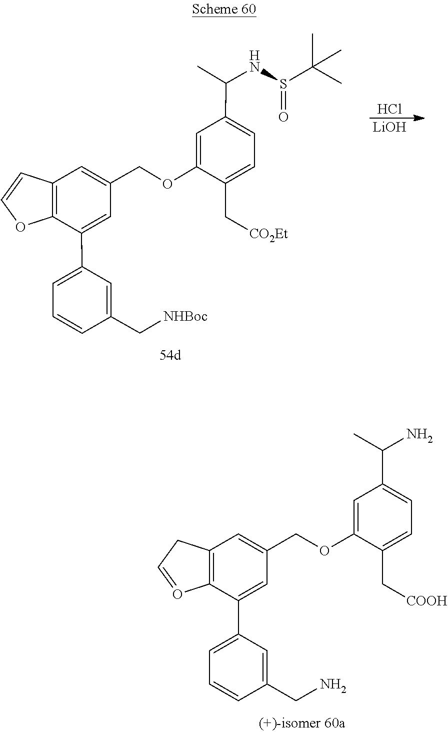

### Preparation of (+)-2-(4-(1-aminoethyl)-2-((7-(3-(aminomethyl)phenyl)benzofuran-5-yl)methoxy)phenyl)acetic acid (60a)

To a solution of ethyl 2-(2-((7-(3-(((tert-butoxycarbonyl)amino)methyl)phenyl)benzofuran-5-yl)methoxy)-4-(1-((R)-1,1-dimethylethylsulfinamido)ethyl)phenyl)acetate (54d) (350 mg, 0.528 mmol) in ethanol (5 mL) was added 4 M HCl in dioxane (0.264 mL, 1.056 mmol) and stirred at rt for 1 h. To the reaction mixture was added 2.0 M aqueous LiOH (1.254 mL, 2.508 mmol) and stirred at RT for 16 h. The reaction mixture was acidified with 3 M HCl (1 mL) and diluted with EtOH (2 mL). The mixture was purified by reverse-phase column chromatography [EZ-PREP, C-18 column (100 g), eluting with 0.1% aqueous HCl in H₂O and MeCN from 0-100%] to provide (+)-2-(4-(1-aminoethyl)-2-((7-(3-(aminomethyl)phenyl)benzofuran-5-yl)methoxy)phenyl)acetic acid (60a) HCl salt (148 mg, 68.5% yield) as a white solid; ¹H NMR (300 MHz, DMSO-d₆) δ 8.94-8.32 (m, 6H), 8.12 (d, J=2.2 Hz, 1H), 8.06 (d, J=2.2 Hz, 1H), 7.95 (dt, J=6.8, 2.1 Hz, 1H), 7.80 (d, J=1.5 Hz, 1H), 7.73 (d, J=1.7 Hz, 1H), 7.66-7.54 (m, 2H), 7.50 (d, J=1.6 Hz, 1H), 7.25 (d, J=7.7 Hz, 1H), 7.07 (d, J=2.2 Hz, 1H), 7.03 (dd, J=7.7, 1.5 Hz, 1H), 5.31 (s, 2H), 4.45-4.27 (m, 1H), 4.14 (s, 2H), 3.60 (s, 2H), 1.51 (d, J=6.7 Hz, 3H); MS (ES+): 431 (M+1), (ES−): 429 (M−1); Analysis calculated for C₂₆H₂₆N₂O₄·2HCl·2.25H₂O: C, 57.41; H, 6.02; Cl, 13.04; N, 5.15; Found: C, 57.41; H, 5.84; Cl, 13.21; N, 5.31; Optical rotation [α]_{D}=+5.455 (c=0.11, MeOH).

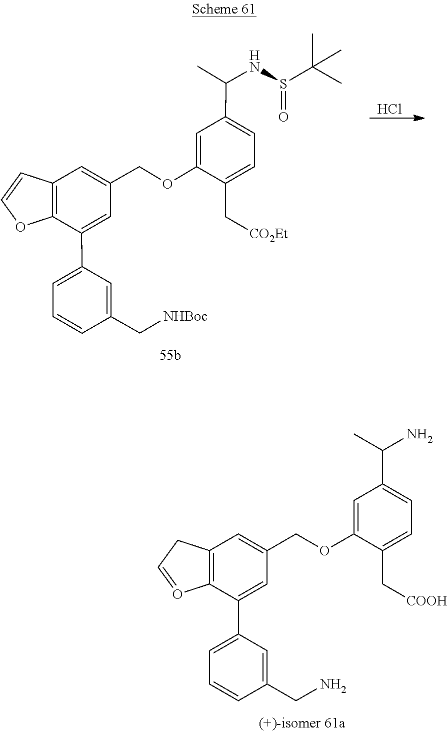

### Preparation of (−)-2-(4-(1-aminoethyl)-2-((7-(3-(aminomethyl)phenyl)benzofuran-5-yl)methoxy)phenyl)acetic acid (61a)

To a solution of ethyl 2-(2-((7-(3-(((tert-butoxycarbonyl)amino)methyl)phenyl)benzofuran-5-yl)methoxy)-4-(1-((S)-1,1-dimethylethylsulfinamido)ethyl)phenyl)acetate (55b) (307 mg, 0.463 mmol) in ethanol (5 mL) was added 4 M HCl in dioxane (0.232 mL, 0.926 mmol) and stirred at rt for 1 h. To the reaction mixture was added 2.0 M aqueous LiOH (1.156 mL, 2.312 mmol) and stirred at rt for 16 h. The reaction mixture was acidified with 3 M HCl (1 mL) and diluted with EtOH (2 mL). The mixture was purified by reverse-phase column chromatography [EZ-PREP, C-18 column (100 g), eluting with 0.1% aqueous HCl in H₂O and MeCN from 0-100%] to provide (−)-2-(4-(1-aminoethyl)-2-((7-(3-(aminomethyl)phenyl)benzofuran-5-yl)methoxy)phenyl)acetic acid (61a) (199 mg, 100% yield) HCl salt as a white solid; ¹H NMR (300 MHz, DMSO-d₆) δ 8.57 (s, 6H, D₂O exchangeable), 8.11 (d, J=2.2 Hz, 1H), 8.05 (d, J=2.1 Hz, 1H), 7.95 (dt, J=6.6, 2.2 Hz, 1H), 7.79 (d, J=1.6 Hz, 1H), 7.72 (d, J=1.7 Hz, 1H), 7.65-7.53 (m, 2H), 7.50 (d, J=1.7 Hz, 1H), 7.24 (d, J=7.7 Hz, 1H), 7.07 (d, J=2.2 Hz, 1H), 7.02 (dd, J=7.7, 1.5 Hz, 1H), 5.30 (s, 2H), 4.43-4.27 (m, 1H), 4.14 (s, 2H), 3.59 (s, 2H), 1.51 (d, J=6.7 Hz, 3H); MS (ES+): 431 (M+1), (ES−): 429 (M−1); Analysis calculated for C₂₆H₂₆N₂O₄·2HCl·2.25H₂O: C, 57.41; H, 6.02; Cl, 13.04; N, 5.15; Found: C, 57.48; H, 5.90; Cl, 13.06; N, 5.14; Optical rotation [α]_{D}=−1.6 (c=0.125, MeOH).

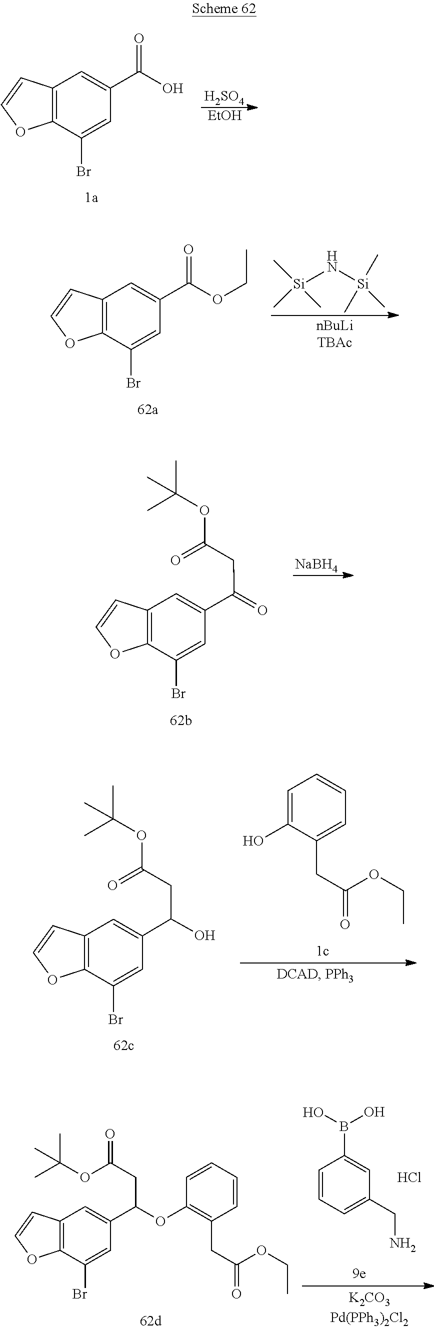

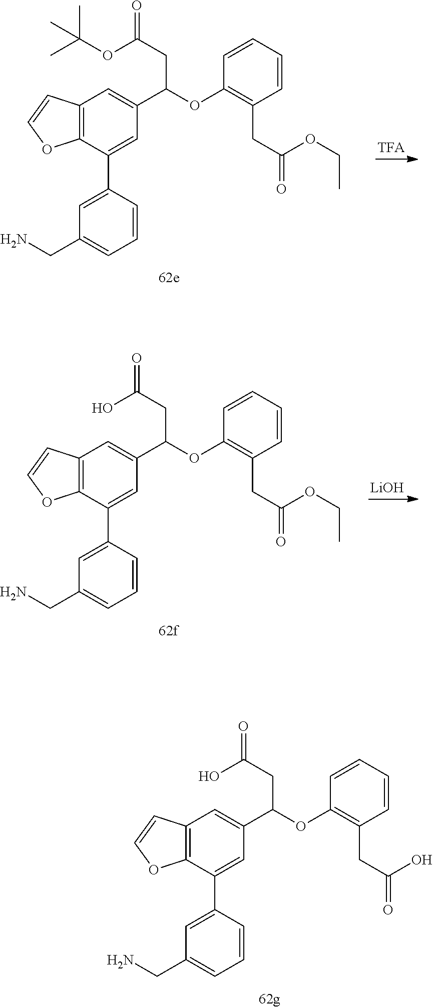

### Preparation of 3-(7-(3-(aminomethyl)phenyl)benzofuran-5-yl)-3-(2-(carboxymethyl)phenoxy)propanoic acid (62g)

### Step-1: Preparation of ethyl 7-bromobenzofuran-5-carboxylate (62a)

Compound 62a was prepared according to procedure reported in Step-2 of scheme 51 from 7-bromobenzofuran-5-carboxylic acid (1a) (5 g, 20.74 mmol) in ethanol (60 mL) using H₂SO₄ (1.11 mL, 20.74 mmol) and heating at 90° C. for 5 h. This gave after workup ethyl 7-bromobenzofuran-5-carboxylate (62a) (5 g, 90% yield) as a white solid; ¹H NMR (300 MHz, DMSO-d₆) δ 8.32 (d, J=1.5 Hz, 1H), 8.25 (d, J=2.2 Hz, 1H), 8.06 (d, J=1.5 Hz, 1H), 7.24 (d, J=2.2 Hz, 1H), 4.34 (q, J=7.1 Hz, 2H), 1.34 (t, J=7.1 Hz, 3H); MS (ES+): 268.90 & 270.90 (M+1).

### Step-2: Preparation of tert-butyl 3-(7-bromobenzofuran-5-yl)-3-oxopropanoate (62b)

To 1,1,1,3,3,3-hexamethyldisilazane (1.87 mL, 8.92 mmol) at 0° C. was added dropwise n-BuLi (3.57 mL, 8.92 mmol), stirred at 0° C. for 10 min and added via syringe into a solution of ethyl 7-bromobenzofuran-5-carboxylate (62a) (1 g, 3.72 mmol) and tert-butyl acetate (0.552 mL, 4.09 mmol) in THF (20 mL) at −10° C. The reaction mixture was stirred between −10° C. to 0° C. for 45 min, poured into ice water and extracted with ethyl acetate. The organic layer was washed with water, 1 M HCl, dried, filtered and concentrated in vacuum. The residue obtained was purified by flash chromatography [silica (40 g), eluting with ethyl acetate in hexane from 0-50%] to give tert-butyl 3-(7-bromobenzofuran-5-yl)-3-oxopropanoate (62b) (1.1 g, 87% yield) as a white solid; ¹H NMR (300 MHz, DMSO-d₆) δ 8.33 (d, J=1.6 Hz, 1H), 8.26 (d, J=2.2 Hz, 1H), 8.10 (d, J=1.6 Hz, 1H), 7.26 (d, J=2.2 Hz, 1H), 4.14 (s, 2H), 1.38 (s, 9H); MS (ES+): 360.90 & 362.90 (M+Na).

### Step-3: Preparation of tert-butyl 3-(7-bromobenzofuran-5-yl)-3-hydroxypropanoate (62c)

Compound 62c was prepared according to procedure reported in Step-3 of scheme 5 from tert-butyl 3-(7-bromobenzofuran-5-yl)-3-oxopropanoate (62b) (0.2 g, 0.59 mmol) in ethanol (10 mL) using sodium borohydride (0.03 g, 0.88 mmol). This gave after workup and purification by flash chromatography [silica (12 g), eluting with ethyl acetate in hexane from 0-50%] tert-butyl 3-(7-bromobenzofuran-5-yl)-3-hydroxypropanoate (62c) (0.15 g, 75% yield) as a white oil; ¹H NMR (300 MHz, DMSO-d₆) δ 8.09 (d, J=2.2 Hz, 1H), 7.63 (d, J=1.7 Hz, 1H), 7.52 (d, J=1.5 Hz, 1H), 7.07 (d, J=2.2 Hz, 1H), 5.61 (d, J=4.8 Hz, 1H), 5.07-4.91 (m, 1H), 3.17 (d, J=5.3 Hz, 2H), 1.33 (s, 9H); MS (ES+): 363.00 & 365.00 (M+Na).

### Step-4: Preparation of tert-butyl 3-(7-bromobenzofuran-5-yl)-3-(2-(2-ethoxy-2-oxoethyl)phenoxy)propanoate (62d)

Compound 62d was prepared according to procedure reported in Step-2 of scheme 1 from tert-butyl 3-(7-bromobenzofuran-5-yl)-3-hydroxypropanoate (62c) (260 mg, 0.76 mmol) in DCM (15 mL) using triphenylphosphine (400 mg, 1.524 mmol) and ethyl 2-(2-hydroxyphenyl)acetate (1c) (165 mg, 0.91 mmol), a solution of bis(4-chlorobenzyl)azodicarboxylate (DCAD) (560 mg, 1.52 mmol) in DCM (5 mL). This gave after workup and purification by flash column chromatography [(silica gel (24 g), eluting with EtOAc in hexanes from 0-30%] tert-butyl 3-(7-bromobenzofuran-5-yl)-3-(2-(2-ethoxy-2-oxoethyl)phenoxy)propanoate (62d)(0.3 g, 78% yield) as a white oil; MS (ES+): 525.10 & 527.10 (M+Na); MS (ES−): 501.00 & 503.00 (M−1).

### Step-5: Preparation of tert-butyl 3-(7-(3-(aminomethyl)phenyl)benzofuran-5-yl)-3-(2-(2-ethoxy-2-oxoethyl)phenoxy)propanoate (62e)

Compound 62e was prepared according to procedure reported in Step-4 of scheme 1 from tert-butyl 3-(7-bromobenzofuran-5-yl)-3-(2-(2-ethoxy-2-oxoethyl)phenoxy)propanoate (62d) (0.3 g, 0.60 mmol) in dioxane (4 mL) using 3-(aminomethyl)phenylboronic acid hydrochloride (9e) (0.22 g, 1.19 mmol), bis(triphenylphosphine)Palladium(II)chloride [Pd(PPh₃)₂Cl₂](0.06 g, 0.09 mmol), a solution of K₂CO₃ (0.25 g, 1.79 mmol) in water (1 mL) and heating at 90° C. for 2 h on oil bath. This gave after workup and purification by flash column chromatography [silica gel (24 g), eluting with DMA-80 in DCM from 0-50%] tert-butyl 3-(7-(3-(aminomethyl)phenyl)benzofuran-5-yl)-3-(2-(2-ethoxy-2-oxoethyl)phenoxy)propanoate (62e) (45 mg, 14% yield) as a yellow oil; MS (ES+): 530.30 (M+1);

### Step-6: Preparation of 3-(7-(3-(aminomethyl)phenyl)benzofuran-5-yl)-3-(2-(2-ethoxy-2-oxoethyl)phenoxy)propanoic acid (62f)

To a solution of tert-butyl 3-(7-(3-(aminomethyl)phenyl)benzofuran-5-yl)-3-(2-(2-ethoxy-2-oxoethyl)phenoxy)propanoate (62e) (45 mg, 0.09 mmol) in DCM (3 mL) was added 2,2,2-trifluoroacetic acid (0.13 mL, 1.70 mmol) and stirred for 2 h at RT. The reaction mixture was concentrated in vacuum and the residue obtained was purified by flash column chromatography [silica (12 g), eluting with DMA-80 in DCM from 0-50%] to afford 3-(7-(3-(aminomethyl)phenyl)benzofuran-5-yl)-3-(2-(2-ethoxy-2-oxoethyl)phenoxy)propanoic acid (62f) (40.2 mg, 100% yield) as a yellow oil; MS (ES+): 474.20 (M+1);

### Step-7: Preparation of 3-(7-(3-(aminomethyl)phenyl)benzofuran-5-yl)-3-(2-(carboxymethyl)phenoxy)propanoic acid (62g)

Compound 62g was prepared according to procedure reported in Step-8 of scheme 1 from 3-(7-(3-(aminomethyl)phenyl)benzofuran-5-yl)-3-(2-(2-ethoxy-2-oxoethyl)phenoxy)propanoic acid (62f) (40 mg, 0.08 mmol) in THE (4 mL), methanol (4 mL) using a solution of lithium hydroxide monohydrate (28.4 mg, 0.68 mmol) in water (1 mL) and stirring for 15 h at room temperature. This gave after workup and purification by reverse-phase column chromatography [C-18 column, eluting with 0.1% aq HCl in water and acetonitrile from 0-100%]3-(7-(3-(aminomethyl)phenyl)benzofuran-5-yl)-3-(2-(carboxymethyl)phenoxy)propanoic acid (62g) (5 mg, 13% yield) HCl salt as a white solid; ¹H NMR (300 MHz, DMSO-d₆) δ 12.29 (s, 2H, D₂O exchangeable), 8.26 (s, 3H, D₂O exchangeable), 8.09 (d, J=2.2 Hz, 1H), 8.03-7.94 (m, 1H), 7.91 (d, J=7.8 Hz, 1H), 7.74 (s, 1H), 7.65-7.47 (m, 3H), 7.16 (d, J=7.5 Hz, 1H), 7.10-6.98 (m, 2H), 6.87-6.75 (m, 2H), 5.95-5.68 (m, 1H), 4.14 (s, 2H), 3.70-3.49 (m, 2H), 2.98-2.81 (m, 2H); MS (ES+): 446.15 (M+1); MS (ES−): 444.10 (M−1).

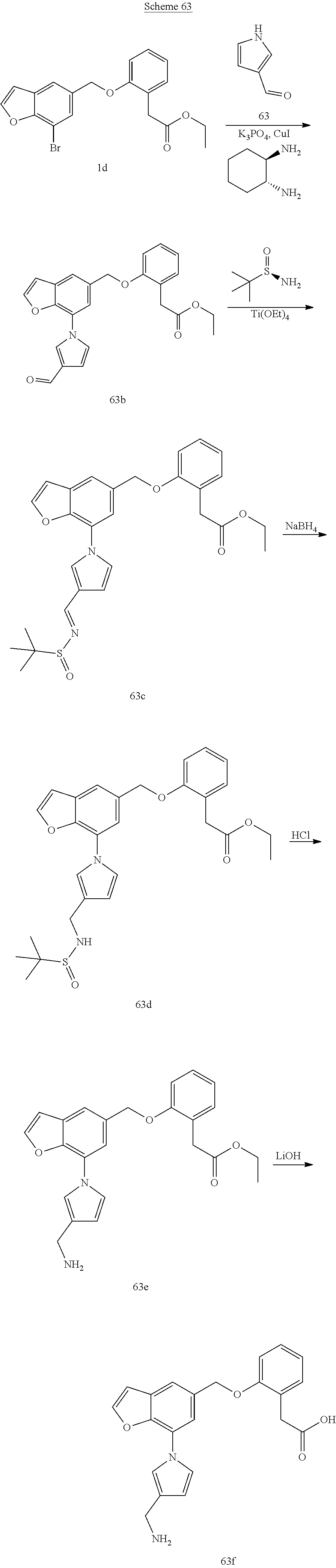

### Preparation of 2-(2-((7-(3-(aminomethyl)-1H-pyrrol-1-yl)benzofuran-5-yl)methoxy)phenyl)acetic acid (63f)

### Step-1: Preparation of ethyl 2-(2-((7-(3-formyl-1H-pyrrol-1-yl)benzofuran-5-yl)methoxy)phenyl)acetate (63b)

To a stirred solution of 1H-pyrrole-3-carbaldehyde (63a) (0.212 g, 2.227 mmol) and ethyl 2-(2-((7-bromobenzofuran-5-yl)methoxy)phenyl)acetate (1d) (1.3 g, 3.34 mmol) in dioxane (15 mL) was added K₃PO₄ (1.418 g, 6.68 mmol), copper(I) iodide (0.424 g, 2.227 mmol), (1R,2R)-cyclohexane-1,2-diamine (0.267 mL, 2.227 mmol) and the mixture was heated at 130° C. for 4 h in a microwave. The reaction mixture was diluted with water, extracted with ethyl acetate (3×100 mL), washed with water (50 mL), brine (30 mL), dried, filtered and concentrated in vacuo. The residue obtained was purified using flash column chromatography [silica gel (40 g), eluting with EtOAc in hexanes from 0-50%] to give ethyl 2-(2-((7-(3-formyl-1H-pyrrol-1-yl)benzofuran-5-yl)methoxy)phenyl)acetate (63b) (259 mg, 29% yield) as a white oil; MS (ES+): 426.1 (M+Na).

### Step-2: Preparation of ethyl 2-(2-((7-(3-(((tert-butylsulfinyl)imino)methyl)-1H-pyrrol-1-yl)benzofuran-5-yl)methoxy)phenyl)acetate (63c)

Compound 63c was prepared according to procedure reported in step-5 of scheme 1, from ethyl 2-(2-((7-(3-formyl-1H-pyrrol-1-yl)benzofuran-5-yl)methoxy)phenyl)acetate (63b) (259 mg, 0.642 mmol) in THE (5 mL) using (R)-2-methylpropane-2-sulfinamide (156 mg, 1.284 mmol), tetraethoxytitanium (0.269 mL, 1.284 mmol) and stirring at room temperature for 18 h. This gave after workup and purification by flash column chromatography [silica gel (40 g), eluting with ethyl acetate in hexanes from 0 to 20%]ethyl 2-(2-((7-(3-(((tert-butylsulfinyl)imino)methyl)-1H-pyrrol-1-yl)benzofuran-5-yl)methoxy)phenyl)acetate (63c) (319 mg, 98% yield) as brown syrup; MS (ES+): 507.2 (M+1).

### Step-3: Preparation of ethyl 2-(2-((7-(3-((1,1-dimethylethylsulfinamido)methyl)-1H-pyrrol-1-yl)benzofuran-5-yl)methoxy)phenyl)acetate (63d)

Compound 63d was prepared according to procedure reported in step-3 of scheme 5, from ethyl 2-(2-((7-(3-(((tert-butylsulfinyl)imino)methyl)-1H-pyrrol-1-yl)benzofuran-5-yl)methoxy)phenyl)acetate (63c) (402 mg, 0.794 mmol) in THE (10 mL) and water (1 mL) using sodium borohydride (90 mg, 2.381 mmol). This gave after workup and purification using flash column chromatography [silica gel (25 g), eluting with ethyl acetate in hexanes from 0 to 100%]ethyl 2-(2-((7-(3-((1,1-dimethylethylsulfinamido)methyl)-1H-pyrrol-1-yl)benzofuran-5-yl)methoxy)phenyl)acetate (63d) (55 mg, 14% yield) as a white solid; MS (ES+): 531.2 (M+Na).

### Step-4: Preparation of ethyl 2-(2-((7-(3-(aminomethyl)-1H-pyrrol-1-yl)benzofuran-5-yl)methoxy)phenyl)acetate (63e)

Compound 63e was prepared according to procedure reported in step-7 of scheme 1, from ethyl 2-(2-((7-(3-((1,1-dimethylethylsulfinamido)methyl)-1H-pyrrol-1-yl)benzofuran-5-yl)methoxy)phenyl)acetate (63d) (55 mg, 0.108 mmol) in THE (1 mL) using a 2M solution of HCl (0.162 mL, 0.324 mmol) and stirring at RT for 4 h. Reaction mixture was concentrated to dryness to afford ethyl 2-(2-((7-(3-(aminomethyl)-1H-pyrrol-1-yl)benzofuran-5-yl)methoxy)phenyl)acetate (63e) (43 mg) and used as such for the next step; MS (ES+): 427.20 (M+Na).

### Step-5: Preparation of 2-(2-((7-(3-(aminomethyl)-1H-pyrrol-1-yl)benzofuran-5-yl)methoxy)phenyl)acetic acid (63f)

Compound 63f was prepared according to procedure reported in step-8 of scheme 1, from ethyl 2-(2-((7-(3-(aminomethyl)-1H-pyrrol-1-yl)benzofuran-5-yl)methoxy)phenyl)acetate (63e) (43 mg, 0.106 mmol) in THE (50 mL), methanol (5 mL) and water (5 mL) using lithium hydroxide monohydrate (10.18 mg, 0.425 mmol) and stirring for 10 h at room temperature. This gave after workup and purification by reverse-phase column chromatography [C18 column (40 g), eluting with ACN in water (containing 0.1% HCl) from 0-100%]2-(2-((7-(3-(aminomethyl)-1H-pyrrol-1-yl)benzofuran-5-yl)methoxy)phenyl)acetic acid (63f) (3 mg, 8% yield) HCl salt as a light brown solid; ¹H NMR (300 MHz, Methanol-d4) δ 7.89 (d, J=2.2 Hz, 1H), 7.65 (s, 2H), 7.55 (d, 1H), 7.53 (t, J=2.7 Hz, 1H), 7.24 (s, 1H), 7.21 (s, 1H), 7.06 (s, 1H), 7.03 (s, 1H), 6.97 (d, J=2.2 Hz, 1H), 6.92 (t, J=7.6 Hz, 1H), 5.26 (s, 2H), 4.08 (s, 2H), 3.67 (s, 2H); MS (ES+): 360.1 (M−NH₂).

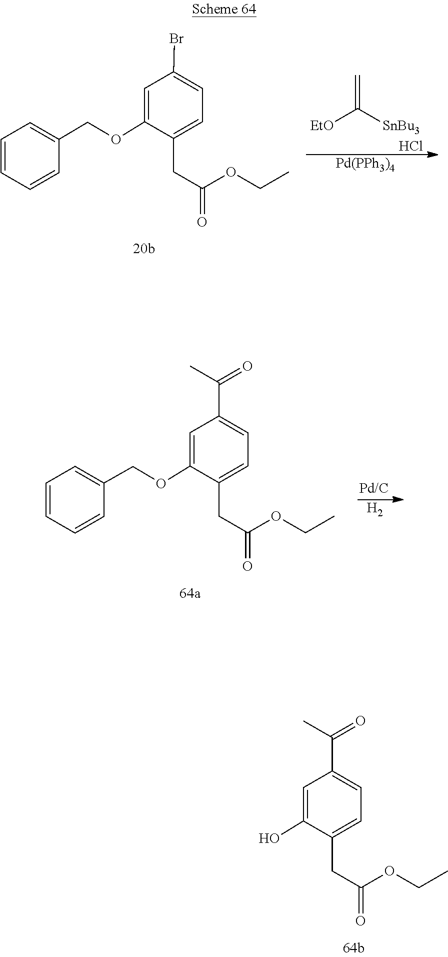

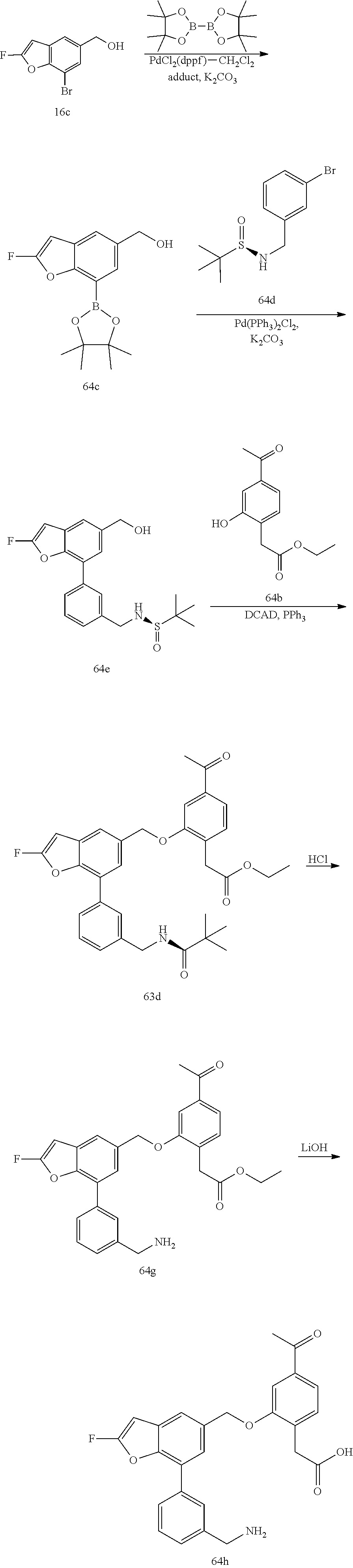

### Preparation of 2-(4-acetyl-2-((7-(3-(aminomethyl)phenyl)-2-fluorobenzofuran-5-yl)methoxy)phenyl)acetic acid (64h)

### Step-1: Preparation of ethyl 2-(4-acetyl-2-(benzyloxy)phenyl)acetate (64a)

Compound 64a was prepared according to procedure reported in step-6 of scheme 12, from ethyl 2-(2-(benzyloxy)-4-bromophenyl)acetate (20b) (12.5 g, 35.8 mmol) in toluene (120 mL) using tributyl(1-ethoxyvinyl)stannane (15.94 mL, 44.7 mmol), Pd(PPh₃)₄ (4.14 g, 3.58 mmol) and heating at 100° C. for 16 h under nitrogen. This gave after workup and purification by flash column chromatography [silica gel (220 g), eluting with ethyl acetate in hexanes from 0 to 30%]ethyl 2-(4-acetyl-2-(benzyloxy)phenyl)acetate (64a) (7.76 g, 69% yield) as an off white solid; MS (ES+): 313.10 (M+1).

### Step-2: Preparation of ethyl 2-(4-acetyl-2-hydroxyphenyl)acetate (64b)

Compound 64b was prepared according to procedure reported in step-3 of scheme 20, from ethyl 2-(4-acetyl-2-(benzyloxy)phenyl)acetate (64a) (7.6 g, 24.33 mmol) in ethyl acetate (150 mL) using Pd/C (2.071 g, 1.946 mmol) and hydrogenating using a balloon for 4 h at room temperature. This gave after workup and purification by flash column chromatography [silica gel (120 g), eluting with ethyl acetate in hexanes from 0 to 35%]ethyl 2-(4-acetyl-2-hydroxyphenyl)acetate (64b) (3.70 g, 68% yield) as a white solid; ¹H NMR (300 MHz, DMSO-d₆) δ 9.94 (s, 1H), 7.40 (dd, J=7.8, 1.7 Hz, 1H), 7.34 (d, J=1.7 Hz, 1H), 7.27 (d, J=7.8 Hz, 1H), 4.07 (q, J=7.1 Hz, 2H), 3.62 (s, 2H), 2.52 (s, 3H), 1.18 (t, J=7.1 Hz, 3H).

### Step-3: Preparation of (2-fluoro-7-(4,4,5,5-tetramethyl-1,3,2-dioxaborolan-2-yl)benzofuran-5-yl)methanol (64c)

Compound 64c was prepared according to procedure reported in step-3 of scheme 1, from (7-bromo-2-fluorobenzofuran-5-yl)methanol (16c) (1.00 g, 4.08 mmol) in anhydrous dioxane (30 mL) using bis(pinacolato)diboron (1.554 g, 6.12 mmol), potassium acetate (1.202 g, 12.24 mmol), PdCl₂(dppf)-CH₂Cl₂ adduct (0.333 g, 0.408 mmol) and heating at 90° C. for 18 h under an argon atmosphere. This gave after workup and purification by flash column chromatography [silica gel (40 g), eluting with EtOAc in hexane from 0-35%](2-fluoro-7-(4,4,5,5-tetramethyl-1,3,2-dioxaborolan-2-yl)benzofuran-5-yl)methanol (64c) (1.16 g, 97% yield); ¹H NMR (300 MHz, DMSO-d₆) δ 7.63 (d, J=1.8 Hz, 1H), 7.55 (d, J=1.8 Hz, 1H), 6.31 (d, J=6.4 Hz, 1H), 5.26 (q, J=6.0 Hz, 1H), 4.56 (d, J=5.8 Hz, 2H), 1.34 (s, 12H).

### Step-4: Preparation of N-(3-(2-fluoro-5-(hydroxymethyl)benzofuran-7-yl)benzyl)-2-methylpropane-2-sulfinamide (64e)

Compound 64e was prepared according to the procedure reported in step-4 of scheme 1, from (2-fluoro-7-(4,4,5,5-tetramethyl-1,3,2-dioxaborolan-2-yl)benzofuran-5-yl)methanol (64c) (1 g, 3.42 mmol) in dioxane (20 mL) using (R)—N-(3-bromobenzyl)-2-methylpropane-2-sulfinamide (64d) (0.994 g, 3.42 mmol), Pd(PPh₃)₂Cl₂ (0.360 g, 0.514 mmol), K₂CO₃ (1.419 g, 10.27 mmol) in water (2.5 mL) and heating under a nitrogen atmosphere at 100° C. for 16 h. This gave after workup and purification by flash column chromatography [silica gel (40 g), eluting with EtOAc/MeOH (9:1) in hexanes]N-(3-(2-fluoro-5-(hydroxymethyl)benzofuran-7-yl)benzyl)-2-methylpropane-2-sulfinamide (64e) (598 mg, 46.5% yield) as a pale yellow oil; MS (ES+): 376.10 (M+1).

### Step-5: Preparation of ethyl 2-(4-acetyl-2-((7-(3-((1,1-dimethylethylsulfinamido)methyl)phenyl)-2-fluorobenzofuran-5-yl)methoxy)phenyl)acetate (64f)

Compound 64f was prepared according to procedure reported in step-2 of scheme 1, from N-(3-(2-fluoro-5-(hydroxymethyl)benzofuran-7-yl)benzyl)-2-methylpropane-2-sulfinamide (64e) (450 mg, 1.199 mmol) in DCM (15 mL) using triphenylphosphine (472 mg, 1.798 mmol), ethyl 2-(4-acetyl-2-hydroxyphenyl)acetate (64b) (266 mg, 1.199 mmol) and a solution of (E)-bis(4-chlorobenzyl) diazene-1,2-dicarboxylate (DCAD, 660 mg, 1.798 mmol) in DCM (15 mL) and stirring at room temperature for 4 h. This gave after workup and purification by flash column chromatography [silica gel (40 g), eluting with EtOAc/MeOH (9:1) in hexanes]ethyl 2-(4-acetyl-2-((7-(3-((1,1-dimethylethylsulfinamido)methyl)phenyl)-2-fluorobenzofuran-5-yl)methoxy)phenyl)acetate (64f) (490 mg, 0.845 mmol, 70.5% yield) as a clear oil; ¹H NMR (300 MHz, DMSO-d₆) δ 7.83 (s, 1H), 7.70 (d, J=7.6 Hz, 1H), 7.67-7.47 (m, 5H), 7.42 (t, J=8.1 Hz, 2H), 6.47 (d, J=6.4 Hz, 1H), 5.88 (t, J=6.0 Hz, 1H), 5.32 (s, 2H), 4.36-4.22 (m, 2H), 3.94 (q, J=7.1 Hz, 2H), 3.73 (s, 2H), 2.59 (s, 3H), 1.17 (s, 9H), 0.99 (t, J=7.1 Hz, 3H); MS (ES+): 580.20 (M+1).

### Step-6: Preparation of ethyl 2-(4-acetyl-2-((7-(3-(aminomethyl)phenyl)-2-fluorobenzofuran-5-yl)methoxy)phenyl)acetate (64g)

Compound 64g was prepared according to procedure reported in step-7 of scheme 1, from ethyl 2-(4-acetyl-2-((7-(3-((1,1-dimethylethylsulfinamido)methyl)phenyl)-2-fluorobenzofuran-5-yl)methoxy)phenyl)acetate (64f) (470 mg, 0.811 mmol) in THE (35 mL) using 3M aqueous HCl (0.811 mL, 2.432 mmol) and stirring at RT for 2 h. This gave after workup ethyl 2-(4-acetyl-2-((7-(3-(aminomethyl)phenyl)-2-fluorobenzofuran-5-yl)methoxy)phenyl)acetate (64g) and was used as such for next step; MS (ES+): 476.20 (M+1).

### Step-7: Preparation of 2-(4-acetyl-2-((7-(3-(aminomethyl)phenyl)-2-fluorobenzofuran-5-yl)methoxy)phenyl)acetic acid (64h)

Compound 64h was prepared according to procedure reported in step-8 of scheme 1, from ethyl 2-(4-acetyl-2-((7-(3-(aminomethyl)phenyl)-2-fluorobenzofuran-5-yl)methoxy)phenyl)acetate (64g) (190 mg, 0.4 mmol) in THE (3 mL), acetonitrile (1.5 mL) and water (1 mL) using lithium hydroxide monohydrate (28.7 mg, 1.2 mmol) and stirring for 14 h at room temperature. This gave after workup and purification using reverse-phase column chromatography [C18 column (50 g), eluting with ACN in water (containing 0.1% HCl) from 0-70%]2-(4-acetyl-2-((7-(3-(aminomethyl)phenyl)-2-fluorobenzofuran-5-yl)methoxy)phenyl)acetic acid (64h) (58 mg, 32% yield) HCl salt as a white solid; ¹H NMR (300 MHz, DMSO-d₆) δ 8.38 (s, 3H, D₂O exchangeable), 7.97-7.92 (m, 1H), 7.86 (dt, J=7.4, 1.8 Hz, 1H), 7.70 (d, J=1.6 Hz, 1H), 7.65 (d, J=1.6 Hz, 1H), 7.62-7.54 (m, 4H), 7.40 (d, J=8.1 Hz, 1H), 6.46 (d, J=6.4 Hz, 1H), 5.35 (s, 2H), 4.14 (s, 2H), 3.69 (s, 2H), 2.57 (s, 3H); ¹⁹F NMR (282 MHz, DMSO-d₆) δ −111.49; MS (ES+): 448.1 (M+1); Analysis calculated for C₂₆H₂₂FNO₅·HCl·1.25H₂O: C, 61.66; H, 5.08; Cl, 7.00; N, 2.77; Found: C, 61.82; H, 5.06; Cl, 6.74; N, 2.77.

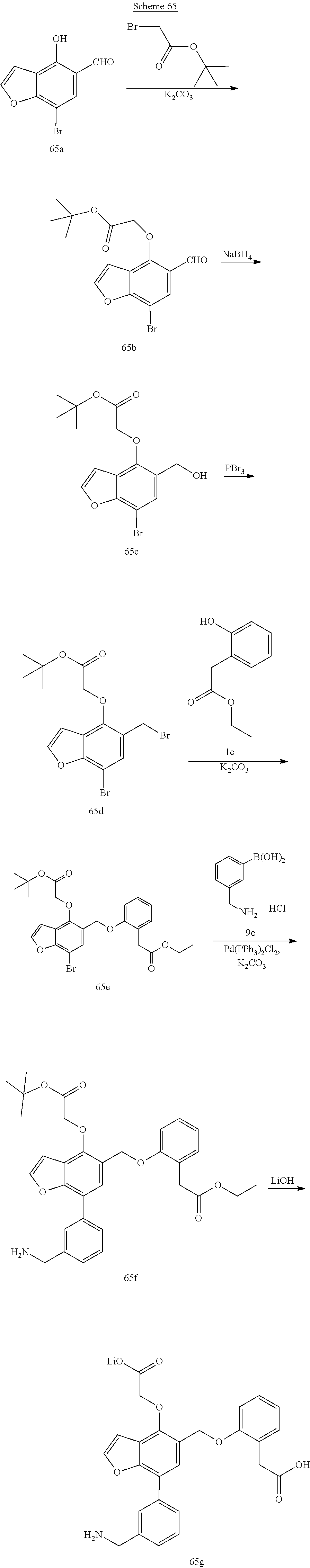

### Preparation of lithium 2-((7-(3-(aminomethyl)phenyl)-5-((2-(carboxymethyl)phenoxy)methyl)benzofuran-4-yl)oxy)acetate (65g)

### Step-1: Preparation of tert-butyl 2-((7-bromo-5-formylbenzofuran-4-yl)oxy)acetate (65b)

Compound 65b was prepared according to the procedure reported in step-4 of scheme 14, from 7-bromo-4-hydroxybenzofuran-5-carbaldehyde (65a) (277 mg, 1.149 mmol; CAS #2173203-52-4) in acetone (6 mL) using tert-butyl 2-bromoacetate (448 mg, 2.298 mmol), K₂CO₃ (476 mg, 3.45 mmol) and stirring overnight at RT. This gave after workup and purification using flash column chromatography [silica gel (12 g), eluting with EtOAc in hexane from 0-100%] tert-butyl 2-((7-bromo-5-formylbenzofuran-4-yl)oxy)acetate (65b) (377 mg, 92% yield) as a white solid; ¹H NMR (300 MHz, DMSO-d₆) δ 10.47 (s, 1H), 8.26 (d, J=2.3 Hz, 1H), 7.82 (s, 1H), 7.46 (d, J=2.3 Hz, 1H), 5.13 (s, 2H), 1.40 (s, 9H).

### Step-2: Preparation of tert-butyl 2-((7-bromo-5-(hydroxymethyl)benzofuran-4-yl)oxy)acetate (65c)

Compound 65c was prepared according to the procedure reported in step-3 of scheme 5, from tert-butyl 2-((7-bromo-5-formylbenzofuran-4-yl)oxy)acetate (65b) (375 mg, 1.056 mmol) in THE (4 mL) and MeOH (4 mL) using sodium borohydride (80 mg, 2.11 mmol) and stirring at RT for 20 min. This gave after workup and purification using flash column chromatography [silica gel (12 g), eluting with EtOAc in hexane from 0-50%] tert-butyl 2-((7-bromo-5-(hydroxymethyl)benzofuran-4-yl)oxy)acetate (65c) (321 mg, 85% yield) as a colorless oil; ¹H NMR (300 MHz, DMSO-d₆) δ 8.07 (d, J=2.3 Hz, 1H), 7.54 (s, 1H), 7.21 (d, J=2.3 Hz, 1H), 5.20 (t, J=5.7 Hz, 1H), 4.87 (s, 2H), 4.65 (d, J=5.6 Hz, 2H), 1.41 (s, 9H).

### Step-3: Preparation of tert-butyl 2-((7-bromo-5-(bromomethyl)benzofuran-4-yl)oxy)acetate (65d)

To a solution of tert-butyl 2-((7-bromo-5-(hydroxymethyl)benzofuran-4-yl)oxy)acetate (65c) (319 mg, 0.893 mmol) in anhydrous diethyl ether (8 mL) was added PBr₃ (0.101 mL, 1.072 mmol) dropwise at 0° C. and allowed to warm to room temperature over 180 min. The reaction mixture was diluted with diethyl ether (20 mL) and quenched carefully with water. The organic layer was separated washed with brine, dried, filtered and concentrated in vacuo to provide tert-butyl 2-((7-bromo-5-(bromomethyl)benzofuran-4-yl)oxy)acetate (65d) (352 mg, 94% yield) as a colorless oil; ¹H NMR (300 MHz, DMSO-d₆) mixture of rotamers δ 8.13 (t, J=2.5 Hz, 1H), 7.67 and 7.57 (s, 1H), 7.28 (t, J=2.3 Hz, 1H), 5.12 and 4.98 (2s, 1H), 4.91 (d, J=17.6 Hz, 2H), 1.41 (2s, 9H).

### Step-4: Preparation of tert-butyl 2-((7-bromo-5-((2-(2-ethoxy-2-oxoethyl)phenoxy)methyl)benzofuran-4-yl)oxy)acetate (65e)

Compound 65e was prepared according to the procedure reported in step-4 of scheme 14, from tert-butyl 2-((7-bromo-5-(bromomethyl)benzofuran-4-yl)oxy)acetate (65d) (0.35 g, 0.833 mmol) in acetone (10 mL) using ethyl 2-(2-hydroxyphenyl)acetate (1c) (0.3 g, 1.666 mmol) and potassium carbonate (461 mg, 3.33 mmol) and stirring at RT for 48 h. This gave after workup and purification using flash column chromatography [silica gel (12 g) eluting with ethyl acetate and hexanes from 0-50%] tert-butyl 2-((7-bromo-5-((2-(2-ethoxy-2-oxoethyl)phenoxy)methyl)benzofuran-4-yl)oxy)acetate (65e) (206 mg, 48% yield) as a pale yellow oil; MS (ES+): 541.1 and 543.1 (M+Na).

### Step-5: Preparation of tert-butyl 2-((7-(3-(aminomethyl)phenyl)-5-((2-(2-ethoxy-2-oxoethyl)phenoxy)methyl)benzofuran-4-yl)oxy)acetate (65f)

Compound 65f was prepared according to the procedure reported in step-7 of scheme 25, from tert-butyl 2-((7-bromo-5-((2-(2-ethoxy-2-oxoethyl)phenoxy)methyl)benzofuran-4-yl)oxy)acetate (65e) (203 mg, 0.391 mmol) in dioxane (5 mL) using 3-(aminomethyl)phenylboronic acid hydrochloride (9e) (110 mg, 0.586 mmol), bis(triphenylphosphine)palladium(II) chloride (41.2 mg, 0.059 mmol), a solution of K₂CO₃ (162 mg, 1.173 mmol) in water (0.5 mL) and stirring at 100° C. for 4.5 h. This gave after workup and purification using flash column chromatography [silica gel (12 g), eluting with DMA-80 in DCM from 0-90%] tert-butyl 2-((7-(3-(aminomethyl)phenyl)-5-((2-(2-ethoxy-2-oxoethyl)phenoxy)methyl)benzofuran-4-yl)oxy)acetate (65f) (134 mg, 63% yield) as a yellow oil; ¹H NMR (300 MHz, DMSO-d₆) δ 8.09 (d, J=2.3 Hz, 1H), 7.76 (s, 1H), 7.66 (d, J=7.6 Hz, 1H), 7.56 (s, 1H), 7.44 (t, J=7.6 Hz, 1H), 7.40-7.32 (m, 1H), 7.31-7.18 (m, 3H), 7.13 (d, J=8.2 Hz, 1H), 6.97-6.86 (m, 1H), 5.32 (s, 2H), 4.94 (s, 2H), 3.94-3.77 (m, 4H), 3.61 (s, 2H), 1.40 (s, 9H), 0.91 (t, J=7.1 Hz, 3H); MS (ES+): 546.3 (M+1).

### Step-6: Preparation of lithium 2-((7-(3-(aminomethyl)phenyl)-5-((2-(carboxymethyl)phenoxy)methyl)benzofuran-4-yl)oxy)acetate (65g)

Compound 65g was prepared according to the procedure reported in step-8 of scheme 1, from tert-butyl 2-((7-(3-(aminomethyl)phenyl)-5-((2-(2-ethoxy-2-oxoethyl)phenoxy)methyl)benzofuran-4-yl)oxy)acetate (65f) (131 mg, 0.240 mmol) in MeOH/THF (6 mL, 1:1) using a solution of lithium hydroxide (50.4 mg, 1.2 mmol) in water (2 mL) and stirring overnight at RT. This gave after workup and purification using reverse phase column chromatography [C18 column (50 g), eluting with ACN in water from 0-100%]lithium 2-((7-(3-(aminomethyl)phenyl)-5-((2-(carboxymethyl)phenoxy)methyl)benzofuran-4-yl)oxy)acetate (65g) (73 mg, 66% yield) Lithium salt as a white solid; ¹H NMR (300 MHz, DMSO-d₆) δ 8.11 (s, 1H), 8.04-7.89 (m, 2H), 7.82 (s, 1H), 7.38 (t, J=7.7 Hz, 1H), 7.29-7.12 (m, 2H), 7.12-6.88 (m, 3H), 6.76 (t, J=7.3 Hz, 1H), 5.41 (s, 2H), 4.56 (s, 2H), 3.94 (s, 2H), 3.41 (s, 2H); MS (ES+): 462.2 (M+1); (ES−): 460.1 (M−1); Analysis calculated for: C₂₆H₂₂LiNO₇·0.1 HCl·2.25H₂O: C, 61.04; H, 5.24; N, 2.74; Cl, 0.69; Found: C, 60.98; H, 4.98; N, 2.74; Cl, 0.64.

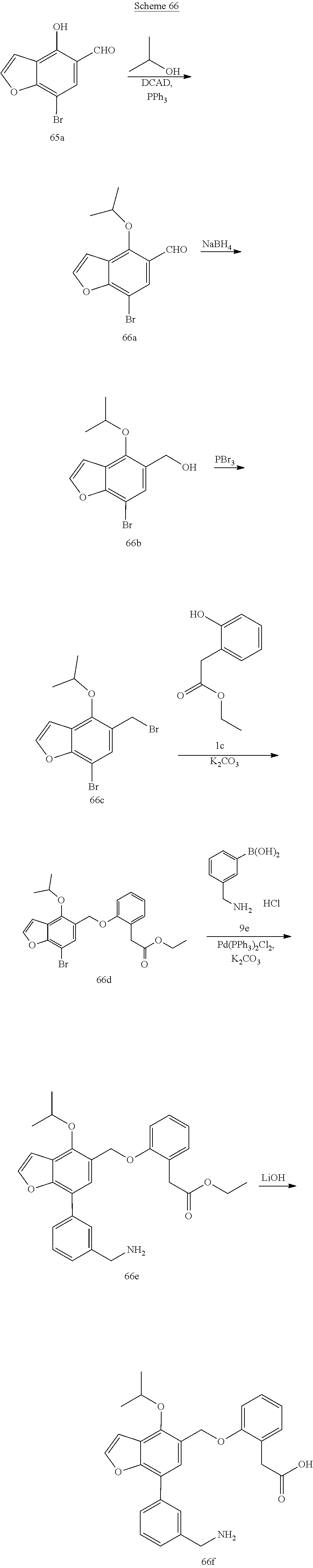

### Preparation of 2-(2-((7-(3-(aminomethyl)phenyl)-4-isopropoxybenzofuran-5-yl)methoxy)phenyl)acetic acid (66f)

### Step-1: Preparation of 7-bromo-4-isopropoxybenzofuran-5-carbaldehyde (66a)

Compound 66a was prepared according to procedure reported in step-2 of scheme 1, from 7-bromo-4-hydroxybenzofuran-5-carbaldehyde (65a) (159 mg, 0.660 mmol) in DCM (6 mL) using propan-2-ol (79 mg, 1.319 mmol), triphenylphosphine (346 mg, 1.319 mmol), a solution of (E)-bis(4-chlorobenzyl) diazene-1,2-dicarboxylate (DCAD, 484 mg, 1.319 mmol) in DCM (2 mL) and stirring at room temperature for 1 h. This gave after workup and purification using flash column chromatography [silica gel (12 g), eluting with EtOAc in hexanes from 0-50%]7-bromo-4-isopropoxybenzofuran-5-carbaldehyde (66a) (48 mg, 26% yield) as a clear oil; ¹H NMR (300 MHz, DMSO-d₆) δ 10.33 (s, 1H), 8.26 (d, J=2.3 Hz, 1H), 7.81 (s, 1H), 7.43 (d, J=2.3 Hz, 1H), 5.05-4.78 (m, 1H), 1.37 (d, J=6.1 Hz, 6H).

### Step-2: Preparation of (7-bromo-4-isopropoxybenzofuran-5-yl)methanol (66b)

Compound 66b was prepared according to the procedure reported in step-3 of scheme 5, from 7-bromo-4-isopropoxybenzofuran-5-carbaldehyde (66a) (318 mg, 1.123 mmol) in THE (3 mL) and MeOH (3 mL) using sodium borohydride (127 mg, 3.37 mmol) and stirring at RT for 30 min. This gave after workup and purification using flash column chromatography [silica gel (12 g), eluting with EtOAc in hexane from 0-70%](7-bromo-4-isopropoxybenzofuran-5-yl)methanol (66b) (289 mg, 90% yield) as a white solid; ¹H NMR (300 MHz, DMSO-d₆) δ 8.06 (d, J=2.3 Hz, 1H), 7.54 (s, 1H), 7.15 (d, J=2.3 Hz, 1H), 5.19 (t, J=5.7 Hz, 1H), 4.66-4.59 (m, 1H), 4.57 (d, J=5.6 Hz, 2H), 1.27 (d, J=6.1 Hz, 6H).

### Step-3: Preparation of 7-bromo-5-(bromomethyl)-4-isopropoxybenzofuran (66c)

Compound 66c was prepared according to the procedure reported in step-3 of scheme 65, from (7-bromo-4-isopropoxybenzofuran-5-yl)methanol (66b) (198 mg, 0.694 mmol) in anhydrous diethyl ether (8 mL) using PBr₃ (0.068 mL, 0.724 mmol). This gave after work up 7-bromo-5-(bromomethyl)-4-isopropoxybenzofuran (66c) (239 mg, 99% yield) as a white solid; ¹H NMR (300 MHz, DMSO-d₆) δ 8.13 (d, J=2.3 Hz, 1H), 7.66 (s, 1H), 7.27 (d, J=2.3 Hz, 1H), 4.88 (p, J=6.0 Hz, 1H), 4.76 (s, 2H), 1.34 (d, J=6.1 Hz, 6H).

### Step-4: Preparation of ethyl 2-(2-((7-bromo-4-isopropoxybenzofuran-5-yl)methoxy)phenyl)acetate (66d)

Compound 66d was prepared according to the procedure reported in step-4 of scheme 14, from 7-bromo-5-(bromomethyl)-4-isopropoxybenzofuran (66c) (237 mg, 0.681 mmol) in acetone (10 mL) using ethyl 2-(2-hydroxyphenyl)acetate (1c) (245 mg, 1.362 mmol), potassium carbonate (376 mg, 2.72 mmol) and stirring overnight at RT followed by heating to reflux for 2 h. This gave after workup and purification using flash column chromatography [silica gel (12 g) eluting with ethyl acetate in hexanes from 0-50%]ethyl 2-(2-((7-bromo-4-isopropoxybenzofuran-5-yl)methoxy)phenyl)acetate (66d) (194 mg, 64% yield) as a yellow oil; ¹H NMR (300 MHz, DMSO-d₆) δ 8.12 (d, J=2.3 Hz, 1H), 7.55 (s, 1H), 7.33-7.19 (m, 3H), 7.10 (d, J=8.1 Hz, 1H), 6.98-6.85 (m, 1H), 5.12 (s, 2H), 4.70 (p, J=6.1 Hz, 1H), 4.00 (q, J=7.1 Hz, 2H), 3.60 (s, 2H), 1.29 (d, J=6.1 Hz, 6H), 1.06 (t, J=7.1 Hz, 3H).

### Step-5: Preparation of ethyl 2-(2-((7-(3-(aminomethyl)phenyl)-4-isopropoxybenzofuran-5-yl)methoxy)phenyl)acetate (66e)

Compound 66e was prepared according to the procedure reported in step-7 of scheme 25, from ethyl 2-(2-((7-bromo-4-isopropoxybenzofuran-5-yl)methoxy)phenyl)acetate (66d) (95 mg, 0.212 mmol) in dioxane (5 mL) using 3-(aminomethyl)phenylboronic acid hydrochloride (9e) (48.1 mg, 0.319 mmol), bis(triphenylphosphine)palladium(II) chloride (22.36 mg, 0.032 mmol), a solution of K₂CO₃ (88 mg, 0.637 mmol) in water (0.5 mL) and stirring at 100° C. for 4.5 h. This gave after workup and purification using flash column chromatography [silica gel (12 g), eluting with DMA-80 in DCM from 0-70%]ethyl 2-(2-((7-(3-(aminomethyl)phenyl)-4-isopropoxybenzofuran-5-yl)methoxy)phenyl)acetate (66e) (91 mg, 90% yield) as a dark oil; H NMR (300 MHz, DMSO-d₆) δ 8.08 (d, J=2.3 Hz, 1H), 7.77 (s, 1H), 7.71-7.63 (m, 1H), 7.56 (s, 1H), 7.44 (t, J=7.6 Hz, 1H), 7.36 (d, J=7.7 Hz, 1H), 7.31-7.25 (m, 1H), 7.21 (dd, J=7.5, 1.7 Hz, 1H), 7.17-7.11 (m, 2H), 6.91 (t, J=7.4 Hz, 1H), 5.19 (s, 2H), 4.70 (p, J=6.1 Hz, 1H), 3.90-3.82 (m, 2H), 3.81 (s, 2H), 3.59 (s, 2H), 1.32 (d, J=6.0 Hz, 6H), 0.90 (t, J=7.1 Hz, 3H).

### Step-6: Preparation of 2-(2-((7-(3-(aminomethyl)phenyl)-4-isopropoxybenzofuran-5-yl)methoxy)phenyl)acetic acid (66f)

Compound 66f was prepared according to the procedure reported in step-8 of scheme 1, from ethyl 2-(2-((7-(3-(aminomethyl)phenyl)-4-isopropoxybenzofuran-5-yl)methoxy)phenyl)acetate (66e) (89 mg, 0.188 mmol) in MeOH/THF (4 mL, 1:1) using a solution of lithium hydroxide (27 mg, 0.643 mmol) in water (1.5 mL) and stirring at RT for 24 h. This gave after workup and purification using reverse phase column chromatography [C18 column (50 g), eluting with ACN in water from 0-100%]2-(2-((7-(3-(aminomethyl)phenyl)-4-isopropoxybenzofuran-5-yl)methoxy)phenyl)acetic acid (66f) (52 mg, 62% yield) as a white solid; ¹H NMR (300 MHz, DMSO-d₆) δ 8.22 (s, 1H), 8.11-7.97 (m, 2H), 7.87 (s, 1H), 7.43 (t, J=7.7 Hz, 1H), 7.28 (d, J=7.6 Hz, 1H), 7.17-7.02 (m, 3H), 6.87 (d, J=8.2 Hz, 1H), 6.79 (t, J=7.3 Hz, 1H), 5.25 (s, 2H), 4.75-4.65 (m, 1H), 3.97 (s, 2H), 3.38 (s, 2H), 1.37 (d, J=6.0 Hz, 6H); MS (ES+): 446.2 (M+1); (ES−): 444.1 (M−1); Analysis calculated for: C₂₇H₂₇NO₅·0.75H₂O: C, 70.65; H, 6.26; N, 3.05; Found: C, 70.63; H, 6.45; N, 3.08.

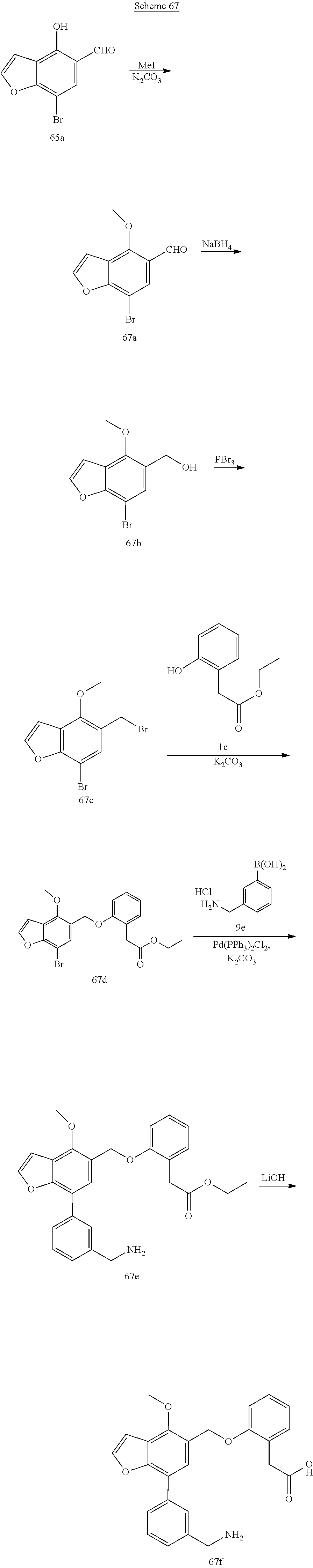

### Preparation of 2-(2-((7-(3-(aminomethyl)phenyl)-4-methoxybenzofuran-5-yl)methoxy)phenyl)acetic acid (67f)

### Step-1: Preparation of 7-bromo-4-methoxybenzofuran-5-carbaldehyde (67a)

Compound 67a was prepared according to the procedure reported in step-4 of scheme 14, from 7-bromo-4-hydroxybenzofuran-5-carbaldehyde (65a) (249 mg, 1.033 mmol) in DMF (5 mL) using methyl iodide (0.388 mL, 6.20 mmol), K₂CO₃ (428 mg, 3.10 mmol) and stirring at 60° C. for 16 h. This gave after workup 7-bromo-4-methoxybenzofuran-5-carbaldehyde (67a) (260 mg, 99% yield) as a white yellow solid; ¹H NMR (300 MHz, DMSO-d₆) δ 10.31 (s, 1H), 8.24 (d, J=2.3 Hz, 1H), 7.79 (s, 1H), 7.61 (d, J=2.4 Hz, 1H), 4.27 (s, 3H); MS (ES+): 255.0, 257.0 (M+H).

### Step-2: Preparation of (7-bromo-4-methoxybenzofuran-5-yl)methanol (67b)

Compound 67b was prepared according to the procedure reported in step-3 of scheme 5, from 7-bromo-4-methoxybenzofuran-5-carbaldehyde (67a) (258 mg, 1.012 mmol) in THE (4 mL) and MeOH (4 mL) using sodium borohydride (115 mg, 3.03 mmol) and stirring at RT for 30 min. This gave after workup and purification using flash column chromatography [silica gel (12 g), eluting with EtOAc in hexane from 0-70%](7-bromo-4-methoxybenzofuran-5-yl)methanol (67b) (236 mg, 91% yield) as a white solid; ¹H NMR (300 MHz, DMSO-d₆) δ 8.06 (d, J=2.3 Hz, 1H), 7.51 (s, 1H), 7.33 (d, J=2.3 Hz, 1H), 5.17 (t, J=5.7 Hz, 1H), 4.55 (d, J=5.7 Hz, 2H), 4.04 (s, 3H).

### Step-3: Preparation of 7-bromo-5-(bromomethyl)-4-methoxybenzofuran (67c)

Compound 67c was prepared according to the procedure reported in step-3 of scheme 65, from (7-bromo-4-methoxybenzofuran-5-yl)methanol (67b) (232 mg, 0.902 mmol) in anhydrous diethyl ether (8 mL) using PBr₃ (0.102 mL, 1.083 mmol). This gave after workup 7-bromo-5-(bromomethyl)-4-methoxybenzofuran (67c) (289 mg, 100% yield) as a white crystals; ¹H NMR (300 MHz, DMSO-d₆) δ 8.13 (d, J=2.4 Hz, 1H), 7.66 (s, 1H), 7.43 (d, J=2.4 Hz, 1H), 4.75 (s, 2H), 4.17 (s, 3H).

### Step-4: Preparation of ethyl 2-(2-((7-bromo-4-methoxybenzofuran-5-yl)methoxy)phenyl)acetate (67d)

Compound 67d was prepared according to the procedure reported in step-4 of scheme 14, from 7-bromo-5-(bromomethyl)-4-methoxybenzofuran (67c) (288 mg, 0.900 mmol) in acetone (10 mL) using ethyl 2-(2-hydroxyphenyl)acetate (1c) (324 mg, 1.800 mmol), potassium carbonate (498 mg, 3.60 mmol) and stirring overnight at RT. This gave after workup and purification using flash column chromatography [silica gel (12 g) eluting with ethyl acetate and hexanes from 0-50%]ethyl 2-(2-((7-bromo-4-methoxybenzofuran-5-yl)methoxy)phenyl)acetate (67d) (271 mg, 72% yield) as a yellow oil; ¹H NMR (300 MHz, DMSO-d₆) δ 8.12 (d, J=2.3 Hz, 1H), 7.55 (s, 1H), 7.41 (d, J=2.3 Hz, 1H), 7.31-7.20 (m, 2H), 7.05 (d, J=1.7 Hz, 1H), 6.93 (td, J=7.4, 1.1 Hz, 1H), 5.11 (s, 2H), 4.10 (s, 3H), 4.02-3.94 (m, 2H), 3.60 (s, 2H), 1.06 (t, J=7.1 Hz, 3H).

### Step-5: Preparation of ethyl 2-(2-((7-(3-(aminomethyl)phenyl)-4-methoxybenzofuran-5-yl)methoxy)phenyl)acetate (67e)

Compound 67e was prepared according to the procedure reported in step-7 of scheme 25, from ethyl 2-(2-((7-bromo-4-methoxybenzofuran-5-yl)methoxy)phenyl)acetate (67d) (135 mg, 0.322 mmol) in dioxane (5 mL) using 3-(aminomethyl)phenylboronic acid hydrochloride (9e) (91 mg, 0.483 mmol), bis(triphenylphosphine)palladium(II) chloride (33.9 mg, 0.048 mmol), a solution of K₂CO₃ (134 mg, 0.966 mmol) in water (0.5 mL) and stirring at 100° C. for 4 h. This gave after workup and purification using flash column chromatography [silica gel (12 g), eluting with DMA-80 in DCM from 0-70%]ethyl 2-(2-((7-(3-(aminomethyl)phenyl)-4-methoxybenzofuran-5-yl)methoxy)phenyl)acetate (67e) (110 mg, 77% yield) as a dark oil; ¹H NMR (300 MHz, DMSO-d₆) δ 8.08 (d, J=2.3 Hz, 1H), 7.79 (s, 1H), 7.70 (d, J=7.4 Hz, 1H), 7.56 (s, 1H), 7.47 (t, J=7.6 Hz, 1H), 7.40 (d, J=2.0 Hz, 1H), 7.33 (d, J=2.3 Hz, 1H), 7.29 (d, J=7.3 Hz, 1H), 7.25-7.19 (m, 1H), 7.14 (d, J=8.2 Hz, 1H), 6.91 (t, J=7.3 Hz, 1H), 5.19 (s, 2H), 4.12 (s, 3H), 3.91-3.79 (m, 4H), 3.59 (s, 2H), 0.91 (t, J=7.1 Hz, 3H); MS (ES+): 466.2 (M+H).

### Step-6: Preparation of 2-(2-((7-(3-(aminomethyl)phenyl)-4-methoxybenzofuran-5-yl)methoxy)phenyl)acetic acid (67f)

Compound 67f was prepared according to the procedure reported in step-8 of scheme 1, from ethyl 2-(2-((7-(3-(aminomethyl)phenyl)-4-methoxybenzofuran-5-yl)methoxy)phenyl)acetate (67e) (108 mg, 0.242 mmol) in MeOH/THF (4 mL, 1:1) using a solution of lithium hydroxide (40.7 mg, 0.970 mmol) in water (1.5 mL) and stirring overnight at RT. This gave after workup and purification using reverse phase column chromatography [C18 column (50 g), eluting with ACN in water from 0-100%]2-(2-((7-(3-(aminomethyl)phenyl)-4-methoxybenzofuran-5-yl)methoxy)phenyl)acetic acid (67f) (54 mg, 53% yield) as a white solid; ¹H NMR (300 MHz, DMSO-d₆) δ 8.21-8.14 (m, 1H), 8.05 (d, J=2.3 Hz, 1H), 8.04-7.98 (m, 1H), 7.86 (s, 1H), 7.42 (t, J=7.7 Hz, 1H), 7.31 (d, J=2.3 Hz, 1H), 7.26 (d, J=7.5 Hz, 1H), 7.08 (t, J=7.7 Hz, 2H), 6.86 (d, J=8.0 Hz, 1H), 6.83-6.73 (m, 1H), 5.24 (s, 2H), 4.15 (s, 3H), 3.95 (s, 2H), 3.39 (s, 2H); MS (ES+): 418.2 (M+1); (ES−): 416.1 (M−1); Analysis calculated for: C₂₅H₂₃NO₅·0.25 HCl·0.5H₂O: C, 68.94; H, 5.61; N, 3.22; Cl, 2.03; Found: C, 69.09; H, 5.74; N, 3.28; Cl, 1.98.

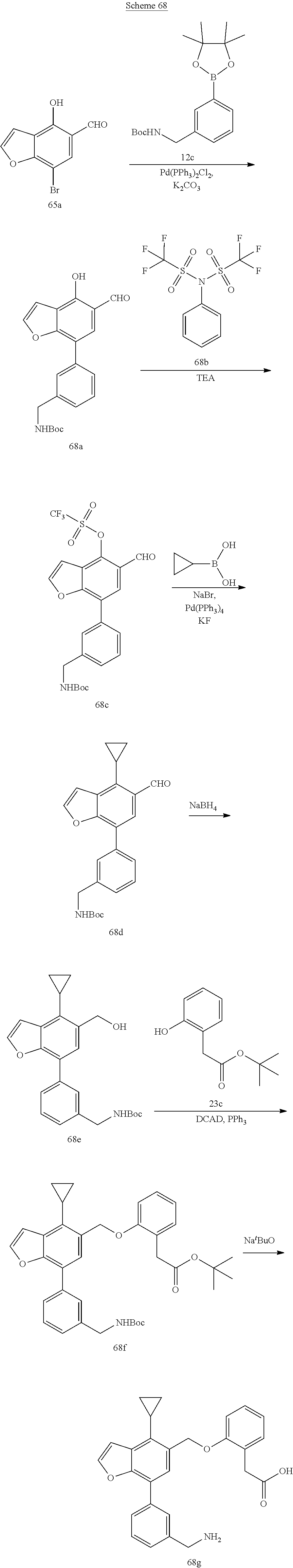

### Preparation of 2-(2-((7-(3-(aminomethyl)phenyl)-4-cyclopropylbenzofuran-5-yl)methoxy)phenyl)acetic acid (68g)

### Step-1: Preparation of tert-butyl 3-(5-formyl-4-hydroxybenzofuran-7-yl)benzylcarbamate (68a)

Compound 68a was prepared according to the procedure reported in step-7 of scheme 25, from 7-bromo-4-hydroxybenzofuran-5-carbaldehyde (65a) (1.86 g, 7.72 mmol) in dioxane (9 mL) using tert-butyl 3-(4,4,5,5-tetramethyl-1,3,2-dioxaborolan-2-yl)benzylcarbamate (12c) (3.60 g, 10.80 mmol), bis(triphenylphosphine)palladium(II) chloride (0.812 g, 1.157 mmol), a solution of K₂CO₃ (3.20 g, 23.15 mmol) in water (1 mL) and stirring at 100° C. for 3 h. This gave after workup and purification using flash column chromatography [silica gel (24 g), eluting with EtOAc in hexane from 0-70%] tert-butyl 3-(5-formyl-4-hydroxybenzofuran-7-yl)benzylcarbamate (68a) (2.21 g, 6.02 mmol, 78% yield) as a yellow oil; MS (ES+): 390.1 (M+Na).

### Step-2: Preparation of 7-(3-(((tert-butoxycarbonyl)amino)methyl)phenyl)-5-formylbenzofuran-4-yl trifluoromethanesulfonate (68c)

To a solution of tert-butyl 3-(5-formyl-4-hydroxybenzofuran-7-yl)benzylcarbamate (68a) (982 mg, 2.67 mmol) in DMF (15 mL) was added 1,1,1-trifluoro-N-phenyl-N-(trifluoromethyl sulfonyl) methanesulfonamide (68b) (974 mg, 2.67 mmol; CAS #37595-74-7), triethylamine (0.745 mL, 5.35 mmol) and stirred overnight at RT. The reaction mixture was diluted with ethyl acetate, washed with water, brine, dried, filtered and concentrated in vacuum. The residue obtained was purified using flash column chromatography [silica (12g), eluting with EtOAc in hexane from 0-60%] to obtain 7-(3-(((tert-butoxycarbonyl)amino)methyl)phenyl)-5-formylbenzofuran-4-yl trifluoromethanesulfonate (68c) (815 mg, 61% yield) as a colorless oil; ¹H NMR (300 MHz, DMSO-d₆) δ 10.23 (s, 1H), 8.42 (d, J=2.4 Hz, 1H), 8.23 (s, 1H), 7.85-7.76 (m, 2H), 7.60-7.47 (m, 2H), 7.40 (d, J=7.7 Hz, 1H), 7.22 (d, J=2.3 Hz, 1H), 4.25 (d, J=6.2 Hz, 2H), 1.41 (s, 9H).

### Step-3: Preparation of tert-butyl 3-(4-cyclopropyl-5-formylbenzofuran-7-yl)benzylcarbamate (68d)

To a mixture of 7-(3-(((tert-butoxycarbonyl)amino)methyl)phenyl)-5-formylbenzofuran-4-yl trifluoromethanesulfonate (68c)(155 mg, 0.310 mmol), cyclopropyl boronic acid (32.0 mg, 0.372 mmol), Pd(PPh₃)₄ (17.93 mg, 0.016 mmol), potassium fluoride (59.5 mg, 1.024 mmol) and sodium bromide (31.9 mg, 0.310 mmol) was added toluene (6 mL) followed by one drop of water and the reaction mixture was stirred at 100° C. for 3 h. The reaction mixture was cooled to RT and the solvent was removed in vacuo. The residue was purified using [silica gel (12 g), eluting with EtOAc in hexane from 0-40%] to give tert-butyl 3-(4-cyclopropyl-5-formylbenzofuran-7-yl)benzylcarbamate (68d) (99 mg, 81% yield) as a yellow oil; ¹H NMR (300 MHz, DMSO-d₆) δ 10.79 (s, 1H), 8.19 (d, J=2.3 Hz, 1H), 7.90 (s, 1H), 7.77-7.66 (m, 2H), 7.50 (q, J=7.7, 6.7 Hz, 2H), 7.37-7.23 (m, 2H), 4.23 (d, J=6.3 Hz, 2H), 2.62-2.54 (m, 1H), 1.41 (s, 9H), 1.28-1.19 (m, 2H), 0.93-0.81 (m, 2H).

### Step-4: Preparation of tert-butyl 3-(4-cyclopropyl-5-(hydroxymethyl)benzofuran-7-yl)benzylcarbamate (68e)

Compound 68e was prepared according to the procedure reported in step-3 of scheme 5, from tert-butyl 3-(4-cyclopropyl-5-formylbenzofuran-7-yl)benzylcarbamate (68d) (267 mg, 0.682 mmol) in THE (3 mL) and MeOH (3 mL) using sodium borohydride (77 mg, 2.046 mmol) and stirring at RT for 30 min. This gave after workup and purification using flash column chromatography [silica gel (12 g), eluting with EtOAc in hexane from 0-70%] tert-butyl 3-(4-cyclopropyl-5-(hydroxymethyl)benzofuran-7-yl)benzylcarbamate (68e) (196 mg, 73% yield) as a pale yellow oil; ¹H NMR (300 MHz, DMSO-d₆) δ 8.00 (d, J=2.3 Hz, 1H), 7.69 (d, J=7.9 Hz, 2H), 7.56 (s, 1H), 7.54-7.40 (m, 2H), 7.26 (d, J=7.6 Hz, 1H), 7.09 (d, J=2.3 Hz, 1H), 5.15 (t, J=5.5 Hz, 1H), 4.83 (d, J=5.4 Hz, 2H), 4.22 (d, J=6.2 Hz, 2H), 2.06 (t, J=5.6 Hz, 1H), 1.40 (s, 9H), 1.12-0.98 (m, 2H), 0.81-0.61 (m, 2H).

### Step-5: Preparation of tert-butyl 2-(2-((7-(3-(((tert-butoxycarbonyl)amino)methyl)phenyl)-4-cyclopropylbenzofuran-5-yl)methoxy)phenyl)acetate (68f)

Compound 68f was prepared according to procedure reported in step-2 of scheme 1, from tert-butyl 3-(4-cyclopropyl-5-(hydroxymethyl)benzofuran-7-yl)benzylcarbamate (68e) (192 mg, 0.488 mmol) in DCM (6 mL) using tert-butyl 2-(2-hydroxyphenyl)acetate (23c) (152 mg, 0.732 mmol), triphenylphosphine (192 mg, 0.732 mmol), a solution of (E)-bis(4-chlorobenzyl) diazene-1,2-dicarboxylate (DCAD, 269 mg, 0.732 mmol) in DCM (2 mL) and stirring at room temperature for 1 h. This gave after workup and purification using flash column chromatography [silica gel (12 g), eluting with EtOAc in hexanes from 0-50%] tert-butyl 2-(2-((7-(3-(((tert-butoxycarbonyl)amino)methyl)phenyl)-4-cyclopropylbenzofuran-5-yl)methoxy)phenyl)acetate (68f) (178 mg, 63% yield) as a clear oil; ¹H NMR (300 MHz, DMSO-d₆) δ 8.06 (d, J=2.3 Hz, 1H), 7.69 (d, J=8.8 Hz, 2H), 7.59 (s, 1H), 7.45 (t, J=7.5 Hz, 2H), 7.34-7.23 (m, 2H), 7.23-7.12 (m, 3H), 6.91 (t, J=7.3 Hz, 1H), 5.38 (s, 2H), 4.21 (d, J=6.2 Hz, 2H), 3.50 (s, 2H), 2.27-2.09 (m, 1H), 1.39 (s, 9H), 1.12 (s, 9H), 1.09-1.01 (m, 2H), 0.82-0.75 (m, 2H).

### Step-6: Preparation of 2-(2-((7-(3-(aminomethyl)phenyl)-4-cyclopropylbenzofuran-5-yl)methoxy)phenyl)acetic acid (68g)

To a solution of tert-butyl 2-(2-((7-(3-(((tert-butoxycarbonyl)amino)methyl)phenyl)-4-cyclopropylbenzofuran-5-yl)methoxy)phenyl)acetate (68f) (65 mg, 0.111 mmol) in THE (4 mL) was added sodium tert-butoxide (121 mg, 1.259 mmol), water (14 mg, 0.777 mmol) and the mixture was heated at 67° C. for 66 h. The solvent was removed in vacuo, the residue obtained was dissolved in water and purified using reverse phase column chromatography [C18 column (50 g), eluting with ACN in water from 0-100%] to provide 2-(2-((7-(3-(aminomethyl)phenyl)-4-cyclopropylbenzofuran-5-yl)methoxy)phenyl)acetic acid (68g) (26 mg, 55% yield) as a white solid; ¹H NMR (300 MHz, DMSO-d₆) δ 8.20 (s, 1H), 8.11-8.01 (m, 2H), 7.90 (s, 1H), 7.46 (t, J=7.7 Hz, 1H), 7.32 (d, J=7.6 Hz, 1H), 7.19-7.06 (m, 3H), 6.92 (d, J=8.4 Hz, 1H), 6.82 (t, J=7.3 Hz, 1H), 5.44 (s, 2H), 3.99 (s, 2H), 3.42 (s, 2H), 2.23-2.06 (m, 1H), 1.19-1.04 (m, 2H), 0.85-0.64 (m, 2H); MS (ES+): 428.2 (M+1); (ES−): 426.2 (M−1).

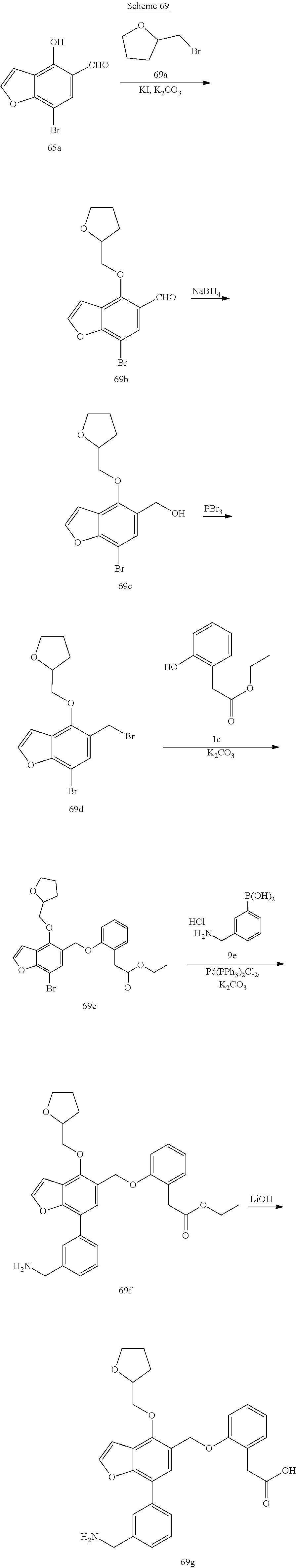

### Preparation of 2-(2-((7-(3-(aminomethyl)phenyl)-4-((tetrahydrofuran-2-yl)methoxy)benzofuran-5-yl)methoxy)phenyl)acetic acid (69g)

### Step-1: Preparation of 7-bromo-4-((tetrahydrofuran-2-yl)methoxy)benzofuran-5-carbaldehyde (69b)

Compound 69b was prepared according to the procedure reported in step-4 of scheme 14, from 7-bromo-4-hydroxybenzofuran-5-carbaldehyde (65a) (254 mg, 1.054 mmol) in DMF (6 mL) using 2-(bromomethyl)tetrahydrofuran (69a) (522 mg, 3.16 mmol; CAS #1192-30-9), potassium iodide (175 mg, 1.054 mmol), K₂CO₃ (437 mg, 3.16 mmol) and stirring at 60° C. for 40 h. This gave after workup 7-bromo-4-((tetrahydrofuran-2-yl)methoxy)benzofuran-5-carbaldehyde (69b) (317 mg, 93% yield) as a yellow solid; MS (ES+): 325.0, 327.0 (M+1).

### Step-2: Preparation of (7-bromo-4-((tetrahydrofuran-2-yl)methoxy)benzofuran-5-yl)methanol (69c)

Compound 69c was prepared according to the procedure reported in step-3 of scheme 5, from 7-bromo-4-((tetrahydrofuran-2-yl)methoxy)benzofuran-5-carbaldehyde (69b) (317 mg, 0.975 mmol) in THE (4 mL) and MeOH (4 mL) using sodium borohydride (111 mg, 2.92 mmol) and stirring at RT for 10 min. This gave after workup and purification using flash column chromatography [silica gel (12 g), eluting with EtOAc in hexane from 0-90%](7-bromo-4-((tetrahydrofuran-2-yl)methoxy)benzofuran-5-yl)methanol (69c) (270 mg, 85% yield) as a colorless oil; ¹H NMR (300 MHz, DMSO-d₆) δ 8.06 (d, J=2.3 Hz, 1H), 7.52 (s, 1H), 7.26 (d, J=2.3 Hz, 1H), 5.16 (t, J=5.7 Hz, 1H), 4.59 (d, J=4.9 Hz, 2H), 4.29-4.19 (m, 1H), 4.19-4.09 (m, 2H), 3.88-3.75 (m, 1H), 3.75-3.63 (m, 1H), 2.03-1.94 (m, 1H), 1.93-1.78 (m, 2H), 1.78-1.63 (m, 1H).

### Step-3: Preparation of 7-bromo-5-(bromomethyl)-4-((tetrahydrofuran-2-yl)methoxy)benzofuran (69d)

Compound 69d was prepared according to the procedure reported in step-3 of scheme 65, from (7-bromo-4-((tetrahydrofuran-2-yl)methoxy)benzofuran-5-yl)methanol (69c) (268 mg, 0.819 mmol) in anhydrous diethyl ether (8 mL) using PBr₃ (0.093 mL, 0.983 mmol). This gave after work up 7-bromo-5-(bromomethyl)-4-((tetrahydrofuran-2-yl)methoxy)benzofuran (69d) (320 mg) as a colorless oil which was used as such for next step.

### Step-4: Preparation of ethyl 2-(2-((7-bromo-4-((tetrahydrofuran-2-yl)methoxy)benzofuran-5-yl)methoxy)phenyl)acetate (69e)

Compound 69e was prepared according to the procedure reported in step-4 of scheme 14, from 7-bromo-5-(bromomethyl)-4-((tetrahydrofuran-2-yl)methoxy)benzofuran (69d) (320 mg, 0.820 mmol) in acetone (10 mL) using ethyl 2-(2-hydroxyphenyl)acetate (1c) (296 mg, 1.641 mmol), potassium carbonate (454 mg, 3.28 mmol) and stirring overnight at RT. This gave after workup and purification using flash column chromatography [silica gel (12 g) eluting with ethyl acetate and hexanes from 0-80%]ethyl 2-(2-((7-bromo-4-((tetrahydrofuran-2-yl)methoxy)benzofuran-5-yl)methoxy)phenyl)acetate (69e) (283 mg, 71% yield) as a pale yellow oil; ¹H NMR (300 MHz, DMSO-d₆) δ 8.12 (d, J=2.3 Hz, 1H), 7.53 (s, 1H), 7.34 (d, J=2.3 Hz, 1H), 7.31-7.19 (m, 2H), 7.08 (d, J=8.1 Hz, 1H), 6.92 (dd, J=7.9, 6.8 Hz, 1H), 5.17 (s, 2H), 4.34-4.26 (m, 1H), 4.26-4.12 (m, 2H), 3.99 (q, J=7.1 Hz, 2H), 3.81-3.63 (m, 2H), 3.61 (s, 2H), 1.98-1.90 (m, 1H), 1.90-1.76 (m, 2H), 1.76-1.61 (m, 1H), 1.06 (t, J=7.1 Hz, 3H).

### Step-5: Preparation of ethyl 2-(2-((7-(3-(aminomethyl)phenyl)-4-((tetrahydrofuran-2-yl)methoxy)benzofuran-5-yl)methoxy)phenyl)acetate (69f)

Compound 69f was prepared according to the procedure reported in step-7 of scheme 25, from ethyl 2-(2-((7-bromo-4-((tetrahydrofuran-2-yl)methoxy)benzofuran-5-yl)methoxy)phenyl)acetate (69e) (140 mg, 0.286 mmol) in dioxane (5 mL) using 3-(aminomethyl)phenylboronic acid hydrochloride (9e) (80 mg, 0.429 mmol), bis(triphenylphosphine)palladium(II) chloride (30.1 mg, 0.043 mmol), a solution of K₂CO₃ (119 mg, 0.858 mmol) in water (0.5 mL) and stirring at 100° C. for 4.5 h. This gave after workup and purification using flash column chromatography [silica gel (12 g), eluting with DMA-80 in DCM from 0-70%]ethyl 2-(2-((7-(3-(aminomethyl)phenyl)-4-((tetrahydrofuran-2-yl)methoxy)benzofuran-5-yl)methoxy)phenyl)acetate (69f) (123 mg, 83% yield) as a dark oil; MS (ES+): 516.2 (M+1).

### Step-6: Preparation of 2-(2-((7-(3-(aminomethyl)phenyl)-4-((tetrahydrofuran-2-yl)methoxy)benzofuran-5-yl)methoxy)phenyl)acetic acid (69g)

Compound 69g was prepared according to the procedure reported in step-8 of scheme 1, from ethyl 2-(2-((7-(3-(aminomethyl)phenyl)-4-((tetrahydrofuran-2-yl)methoxy)benzofuran-5-yl)methoxy)phenyl)acetate (69f) (123 mg, 0.239 mmol) in MeOH/THF (6 mL, 1:1) using a solution of lithium hydroxide (40.0 mg, 0.954 mmol) in water (2 mL) and stirring overnight at RT. This gave after workup and purification using reverse phase column chromatography [C18 column (50 g), eluting with ACN in water from 0-100%]2-(2-((7-(3-(aminomethyl)phenyl)-4-((tetrahydrofuran-2-yl)methoxy)benzofuran-5-yl)methoxy)phenyl)acetic acid (69g) (61 mg, 52% yield) HCl salt as a white solid; ¹H NMR (300 MHz, DMSO-d₆) δ 8.21 (s, J=1.9 Hz, 1H), 8.15-7.99 (m, 2H), 7.90-7.80 (m, 1H), 7.43 (t, J=7.7 Hz, 1H), 7.32-7.21 (m, 2H), 7.15-7.00 (m, 2H), 6.89 (d, J=8.1 Hz, 1H), 6.84-6.74 (m, 1H), 5.42-5.26 (m, 2H), 4.42-4.36 (m, 1H), 4.29-4.24 (m, 2H), 3.97 (s, 2H), 3.90-3.82 (m, 1H), 3.79-3.71 (m, 1H), 3.49-3.27 (m, 2H), 2.15-1.65 (m, 4H); MS (ES+): 488.2 (M+1); (ES−): 486.2 (M−1); Analysis calculated for: C₂₆H₂₁NO₅·1.05HCl·1.1H₂O: C, 64.31; H, 5.03; N, 2.88; Cl, 7.67; Found: C, 64.24; H, 4.96; N, 2.97; Cl, 7.84.

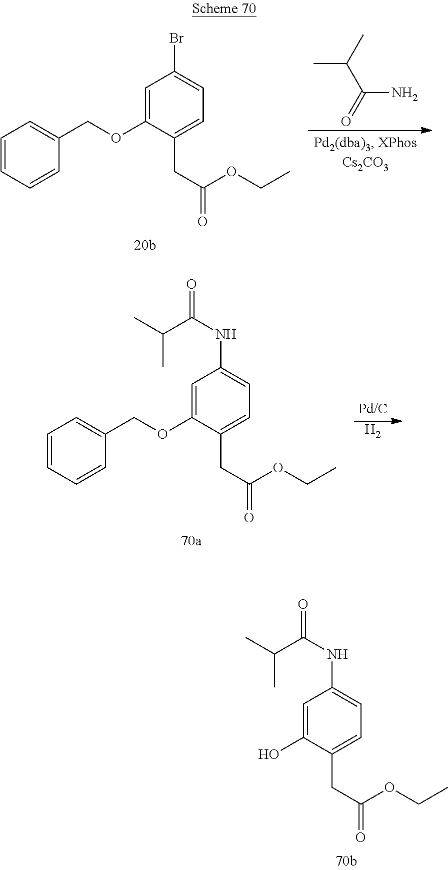

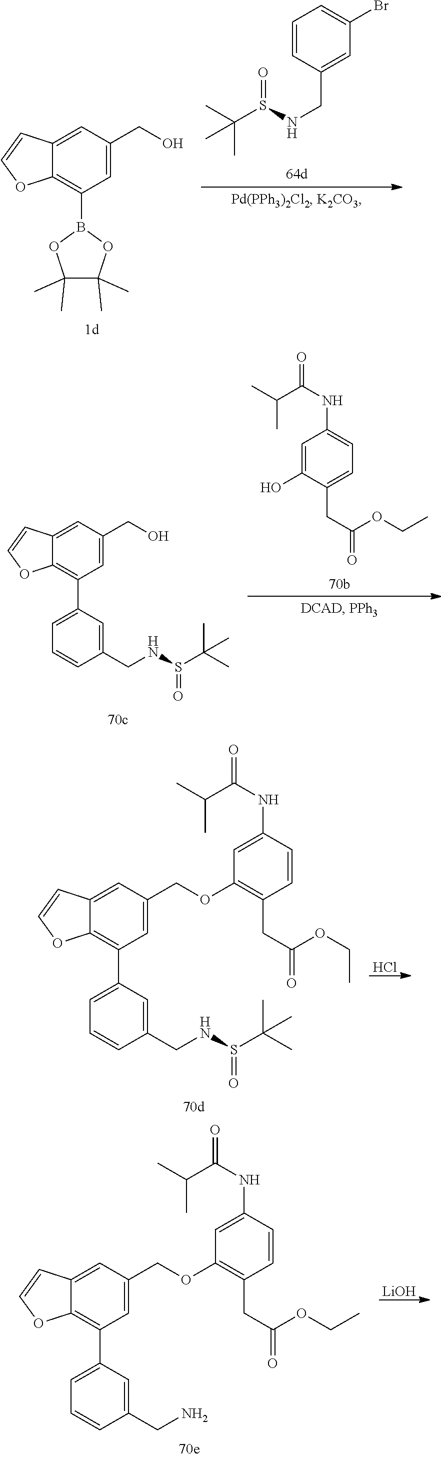

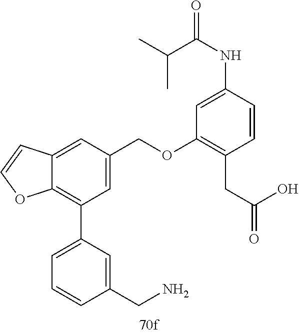 [SMILES: CC(C)C(=O)Nc1ccc(CC(=O)O)c(OCc2cc(-c3cccc(CN)c3)c3occc3c2)c1]

### Preparation of 2-(2-((7-(3-(aminomethyl)phenyl)benzofuran-5-yl)methoxy)-4-isobutyramidophenyl)acetic acid (70f)

### Step-1: Preparation of ethyl 2-(2-(benzyloxy)-4-isobutyramidophenyl)acetate (70a)

Compound 70a was prepared according to the procedure reported in step-1 of scheme 19, from ethyl 2-(2-(benzyloxy)-4-bromophenyl)acetate (20b) (1.5 g, 4.30 mmol) in toluene (100 mL) using isobutyramide (0.374 g, 4.30 mmol), Pd₂(dba)₃ (0.197 g, 0.215 mmol), XPhos (0.205 g, 0.430 mmol), cesium carbonate (1.399 g, 4.30 mmol) and heating under nitrogen at 90° C. for 19 h. This gave after workup and purification by flash column chromatography [silica gel (40 g), eluting with EtOAc in n-heptane from 0-25%]ethyl 2-(2-(benzyloxy)-4-isobutyramidophenyl)acetate (70a) (519 mg, 34% yield); ¹H NMR (300 MHz, DMSO-d₆) δ 9.83 (s, 1H), 7.48 (s, 1H), 7.46-7.29 (m, 5H), 7.10 (s, 2H), 5.03 (s, 2H), 3.99 (q, J=7.1 Hz, 2H), 3.55 (s, 2H), 2.65-2.53 (m, 1H), 1.13-1.06 (m, 9H).

### Step-2: Preparation of ethyl 2-(2-hydroxy-4-isobutyramidophenyl)acetate (70b)

Compound 70b was prepared according to the procedure reported in step-3 of scheme 20, from ethyl 2-(2-(benzyloxy)-4-isobutyramidophenyl)acetate (70a) (500 mg, 1.407 mmol) in ethyl acetate (40 mL) using Pd/C (120 mg, 0.113 mmol) and hydrogenating using a balloon for 16 h at room temperature. This gave after workup and purification by flash column chromatography [silica gel (12 g), eluting with EtOAc in hexanes from 0-40%]ethyl 2-(2-hydroxy-4-isobutyramidophenyl)acetate (70b) (297 mg, 80% yield); MS (ES+): 266.10 (M+1).

### Step-3: Preparation of N-(3-(5-(hydroxymethyl)benzofuran-7-yl)benzyl)-2-methylpropane-2-sulfinamide (70c)

Compound 70c was prepared according to procedure reported in step-4 of scheme 1 from (7-(4,4,5,5-tetramethyl-1,3,2-dioxaborolan-2-yl)benzofuran-5-yl)methanol (1d) (4 g, 14.59 mmol) in dioxane (80 mL) using (R)—N-(3-bromobenzyl)-2-methylpropane-2-sulfinamide (64d) (4.23 g, 14.59 mmol), potassium carbonate (6.05 g, 43.8 mmol), Pd(PPh₃)₂Cl₂ (1.536 g, 2.189 mmol) and heating at 100° C. for 16 h under a nitrogen atmosphere. This gave after workup and purification by flash column chromatography [silica gel (220 g), eluting with EtOAc/MeOH in hexane from 0-60%]N-(3-(5-(hydroxymethyl)benzofuran-7-yl)benzyl)-2-methylpropane-2-sulfinamide (70c) (1.81 g, 35% yield) as a gummy white solid; MS (ES+): 380.10 (M+Na).

### Step-4: Preparation of ethyl 2-(2-((7-(3-((1,1-dimethylethylsulfinamido)methyl)phenyl)benzofuran-5-yl)methoxy)-4-isobutyramidophenyl)acetate (70d)

Compound 70d was prepared according to procedure reported in step-2 of scheme 1 from N-(3-(5-(hydroxymethyl)benzofuran-7-yl)benzyl)-2-methylpropane-2-sulfinamide (70c) (364 mg, 1.018 mmol) in DCM (15 mL) using triphenylphosphine (400 mg, 1.527 mmol), ethyl 2-(2-hydroxy-4-isobutyramidophenyl)acetate (70b) (270 mg, 1.018 mmol) and a solution of (E)-bis(4-chlorobenzyl) diazene-1,2-dicarboxylate (DCAD, 561 mg, 1.527 mmol) in DCM (15 mL) and stirring at room temperature for 4 h. This gave after workup and purification by flash column chromatography [silica gel (40 g), eluting with EtOAc/MeOH (9:1) in hexanes]followed by [silica gel (40 g), eluting with MeOH in DCM from 0-5%]ethyl 2-(2-((7-(3-((1,1-dimethylethylsulfinamido)methyl)phenyl)benzofuran-5-yl)methoxy)-4-isobutyramidophenyl)acetate (70d) (259 mg, 42% yield) as a white solid; MS (ES+): 605.30 (M+1).

### Step-5: Preparation of ethyl 2-(2-((7-(3-(aminomethyl)phenyl)benzofuran-5-yl)methoxy)-4-isobutyramidophenyl)acetate (70e)

Compound 70e was prepared according to procedure reported in step-7 of scheme 1, from ethyl 2-(2-((7-(3-((1,1-dimethylethylsulfinamido)methyl)phenyl)benzofuran-5-yl)methoxy)-4-isobutyramidophenyl)acetate (70d) (250 mg, 0.413 mmol) in THE (20 mL) using 3M aqueous HCl (0.413 mL, 1.24 mmol) and stirring at RT for 2 h. This gave after workup ethyl 2-(2-((7-(3-(aminomethyl)phenyl)benzofuran-5-yl)methoxy)-4-isobutyramidophenyl)acetate (70e) and was used as such for the next step; MS (ES+): 501.25 (M+1).

### Step-6: Preparation of 2-(2-((7-(3-(aminomethyl)phenyl)benzofuran-5-yl)methoxy)-4-isobutyramidophenyl)acetic acid (70f)

Compound 70f was prepared according to the procedure reported in step-8 of scheme 1, from ethyl 2-(2-((7-(3-(aminomethyl)phenyl)benzofuran-5-yl)methoxy)-4-isobutyramidophenyl)acetate (70e) (207 mg, 0.413 mmol) in THE (5 mL), acetonitrile (5 mL) and water (5 mL) using a solution of lithium hydroxide hydrate (106 mg, 2.478 mmol) and stirring at RT for 16 h. This gave after workup and purification using reverse phase column chromatography [C18 column (50 g), eluting with ACN in water (containing 0.1% HCl) from 0-100%]2-(2-((7-(3-(aminomethyl)phenyl)benzofuran-5-yl)methoxy)-4-isobutyramidophenyl)acetic acid (70f) (91 mg, 47% yield) HCl salt as a white solid; ¹H NMR (300 MHz, DMSO-d₆) δ 12.17 (s, 1H, D₂O exchangeable), 9.94 (s, 1H), 8.43 (s, 3H, D₂O exchangeable), 8.11 (d, J=2.2 Hz, 1H), 8.05-8.02 (m, 1H), 7.96 (dt, J=7.2, 1.8 Hz, 1H), 7.77 (d, J=1.6 Hz, 1H), 7.67 (d, J=1.6 Hz, 1H), 7.65-7.52 (m, 3H), 7.14-7.08 (m, 2H), 7.06 (d, J=2.2 Hz, 1H), 5.21 (s, 2H), 4.27-4.00 (m, 2H), 3.53 (s, 2H), 2.69-2.53 (m, 1H), 1.09 (d, J=6.8 Hz, 6H); MS (ES+): 473.2 (M+1); (ES−): 471.2 (M−1); Analysis calculated for C₂₈H₂₈N₂O₅·HCl·1.5H₂O: C, 62.74; H, 6.02; Cl, 6.61; N, 5.23; Found: C, 62.77; H, 6.07; Cl, 6.31; N, 5.17.

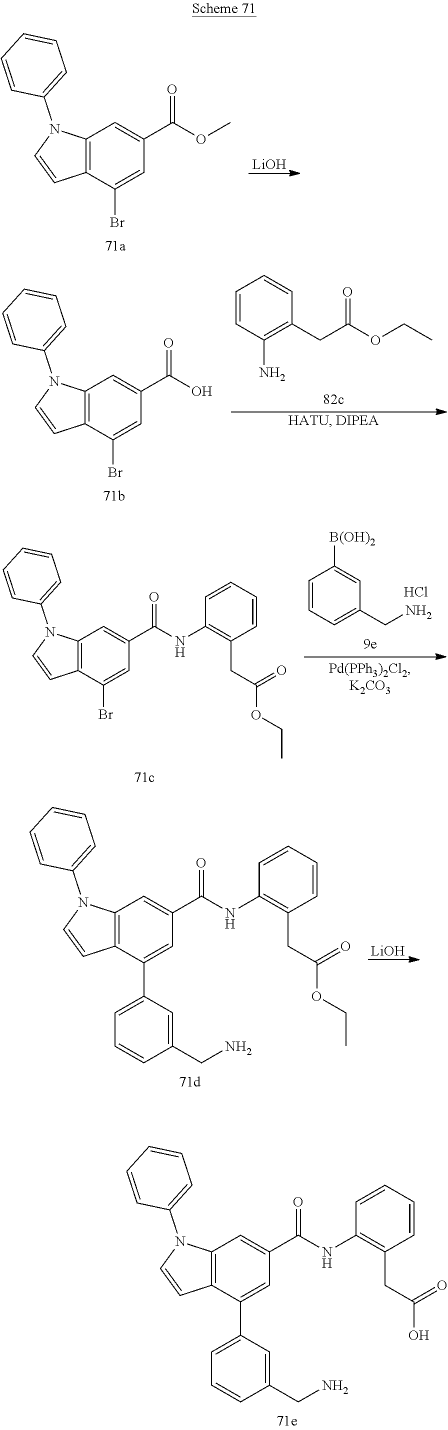

### Preparation of 2-(2-(4-(3-(aminomethyl)phenyl)-1-phenyl-1H-indole-6-carboxamido)phenyl)acetic acid (71e)

### Step-1: Preparation of 4-bromo-1-phenyl-1H-indole-6-carboxylic acid (71b)

Compound 71b was prepared according to procedure reported in step-8 of scheme 1, from methyl 4-bromo-1-phenyl-1H-indole-6-carboxylate (71a) (1 g, 3.03 mmol; CAS #2378274-35-0) in THF (10 mL), MeOH (10 mL) using a solution of lithium hydroxide monohydrate (0.435 g, 18.17 mmol) in water (10 mL) and stirring for 14 h at RT. This gave after workup 4-bromo-1-phenyl-1H-indole-6-carboxylic acid (71b) (922 mg, 96% yield) as a white solid; MS (ES+): 316.0 (M+1).

### Step-2: Preparation of ethyl 2-(2-(4-bromo-1-phenyl-1H-indole-6-carboxamido)phenyl)acetate (71c)

Compound 71c was prepared according to procedure reported in step-2 of scheme 82, from 4-bromo-1-phenyl-1H-indole-6-carboxylic acid (71b) (900 mg, 2.85 mmol) in DMF (25 mL) using ethyl 2-(2-aminophenyl)acetate (82c) (561 mg, 3.13 mmol), HATU (2165 mg, 5.69 mmol), DIPEA (2.479 mL, 14.23 mmol) and stirring at RT for 16 h. This gave after workup and purification using flash column chromatography [silica gel (40 g), eluting with ethyl acetate in hexanes from 0-25%]ethyl 2-(2-(4-bromo-1-phenyl-1H-indole-6-carboxamido)phenyl)acetate (71c) (820 mg, 60% yield) as a sticky brown gum; MS (ES+): 477.10 (M+1).

### Step-3: Preparation of ethyl 2-(2-(4-(3-(aminomethyl)phenyl)-1-phenyl-1H-indole-6-carboxamido)phenyl)acetate (71d)

Compound 71d was prepared according to the procedure reported in step-4 of scheme 1, from ethyl 2-(2-(4-bromo-1-phenyl-1H-indole-6-carboxamido)phenyl)acetate (71c) (800 mg, 1.676 mmol) in dioxane (15 mL) using 3-(aminomethyl)phenylboronic acid hydrochloride (9e) (471 mg, 2.51 mmol), bis(triphenylphosphine)palladium(II) chloride (235 mg, 0.335 mmol), a solution of potassium carbonate (695 mg, 5.03 mmol) in water (1.8 mL) and stirring at 100° C. for 3 h. This gave after workup and purification using flash column chromatography [silica gel (80 g), eluting with MeOH in DCM from 0-10%]ethyl 2-(2-(4-(3-(aminomethyl)phenyl)-1-phenyl-1H-indole-6-carboxamido)phenyl)acetate (71d) (204 mg, 24% yield) as a clear oil; MS (ES+): 504.30 (M+1).

### Step-4: Preparation of 2-(2-(4-(3-(aminomethyl)phenyl)-1-phenyl-1H-indole-6-carboxamido)phenyl)acetic acid (71e)

Compound 71e was prepared according to the procedure reported in step-8 of scheme 1, from ethyl 2-(2-(4-(3-(aminomethyl)phenyl)-1-phenyl-1H-indole-6-carboxamido)phenyl)acetate (71d) (190 mg, 0.377 mmol) in THE (5 mL) and methanol (5 mL) using a solution of lithium hydroxide monohydrate (54.2 mg, 2.264 mmol) in water (5 mL) and stirring at RT for 14 h. This gave after workup and purification using reverse phase column chromatography [C18 column (50 g), eluting with ACN in water (containing 0.1% HCl) from 0-70%]2-(2-(4-(3-(aminomethyl)phenyl)-1-phenyl-1H-indole-6-carboxamido)phenyl)acetic acid (71e) (92 mg, 51% yield) HCl salt as a white solid; ¹H NMR (300 MHz, DMSO-d₆) δ 12.28 (s, 1H, D₂O exchangeable), 10.23 (s, 1H, D₂O exchangeable), 8.47 (s, 3H, D₂O exchangeable), 8.20 (s, 1H), 8.04-7.88 (m, 3H), 7.81 (d, J=7.5 Hz, 1H), 7.74-7.67 (m, 3H), 7.67-7.61 (m, 2H), 7.61-7.53 (m, 1H), 7.53-7.43 (m, 2H), 7.39-7.26 (m, 2H), 7.26-7.15 (m, 1H), 7.01 (d, J=3.3 Hz, 1H), 4.17 (s, 2H), 3.67 (s, 2H); MS (ES+): 476.2 (M+1); (ES−): 474.2 (M−1).

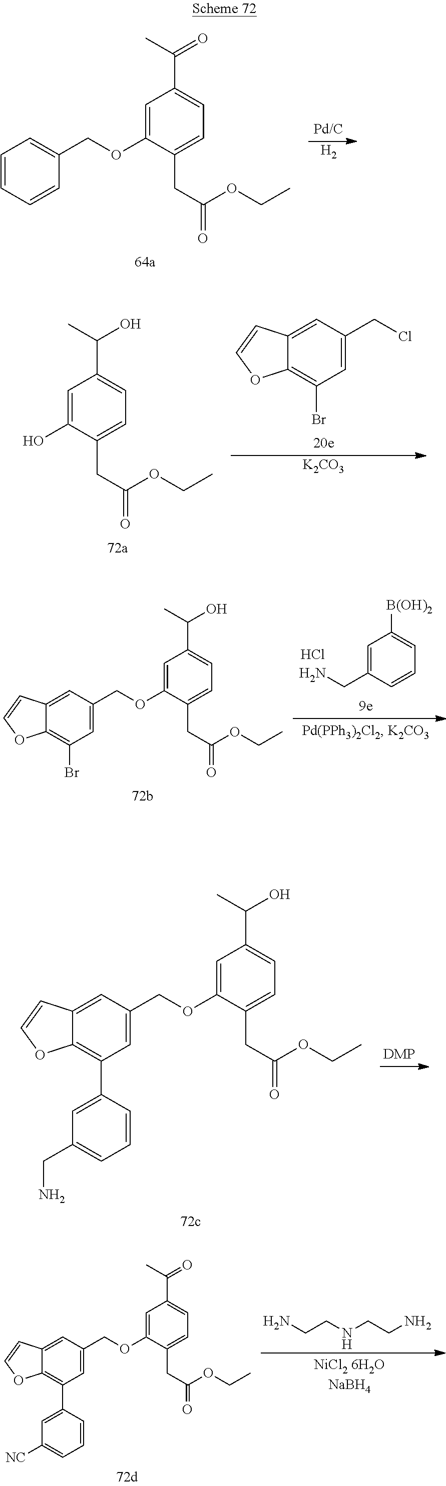

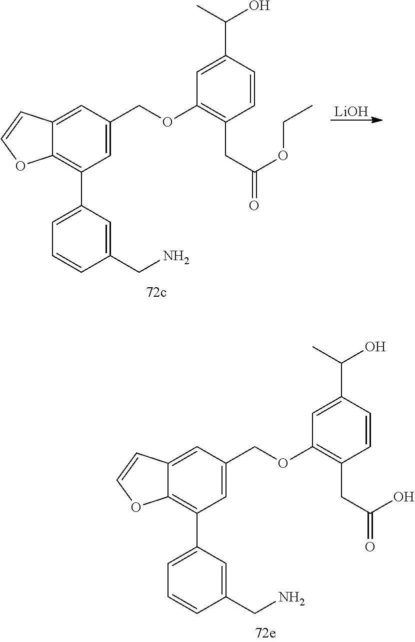

### Preparation of 2-(2-((7-(3-(aminomethyl)phenyl)benzofuran-5-yl)methoxy)-4-(1-hydroxyethyl)phenyl)acetic acid (72e)

### Step-1: Preparation of ethyl 2-(2-hydroxy-4-(1-hydroxyethyl)phenyl)acetate (72a)

Compound 72a was prepared according to procedure reported in step-3 of scheme 20, from ethyl 2-(4-acetyl-2-(benzyloxy)phenyl)acetate (64a) (4.3 g, 13.77 mmol) in ethyl acetate (100 mL) using Pd/C (1.172 g, 1.101 mmol) and hydrogenating using a balloon for 4 h at room temperature. This gave after workup and purification by flash column chromatography [silica gel (80 g), eluting with ethyl acetate in hexanes from 0 to 25%]ethyl 2-(2-hydroxy-4-(1-hydroxyethyl)phenyl)acetate (72a) (2.05 g, 66% yield); ¹H NMR (300 MHz, DMSO-d₆) δ 9.37 (s, 1H), 7.00 (d, J=7.7 Hz, 1H), 6.81 (d, J=1.6 Hz, 1H), 6.68 (dd, J=7.7, 1.6 Hz, 1H), 5.06 (d, J=4.0 Hz, 1H), 4.70-4.50 (m, 1H), 4.11-4.00 (m, 2H), 3.49 (s, 2H), 1.27 (d, J=6.4 Hz, 3H), 1.21-1.12 (m, 3H); MS (ES+): 247.05 (M+Na).

### Step-2: Preparation of ethyl 2-(2-((7-bromobenzofuran-5-yl)methoxy)-4-(1-hydroxyethyl)phenyl)acetate (72b)

Compound 72b was prepared according to the procedure reported in step-4 of scheme 14, from 7-bromo-5-(chloromethyl)benzofuran (20e) (2.189 g, 8.92 mmol) in DMF (50 mL) using ethyl 2-(2-hydroxy-4-(1-hydroxyethyl)phenyl)acetate (72a) (2.00 g, 8.92 mmol), K₂CO₃ (3.70 g, 26.8 mmol) and stirring at RT for 16 h. This gave after workup and purification using flash column chromatography [silica gel (120 g), eluting with EtOAc in hexane from 0-20%]ethyl 2-(2-((7-bromobenzofuran-5-yl)methoxy)-4-(1-hydroxyethyl)phenyl)acetate (72b) (1.95 g, 51% yield); ¹H NMR (300 MHz, DMSO-d₆) δ 8.15 (d, J=2.2 Hz, 1H), 7.73 (d, J=1.4 Hz, 1H), 7.61 (d, J=1.5 Hz, 1H), 7.17-7.10 (m, 2H), 7.08 (d, J=1.5 Hz, 1H), 6.88 (dd, J=7.6, 1.3 Hz, 1H), 5.19-5.14 (m, 3H), 4.75-4.62 (m, 1H), 4.00 (q, J=7.0 Hz, 2H), 3.58 (s, 2H), 1.31 (d, J=6.4 Hz, 3H), 1.07 (t, J=7.1 Hz, 3H); MS (ES+): 455.90 (M+Na).

### Step-3: Preparation of ethyl 2-(2-((7-(3-(aminomethyl)phenyl)benzofuran-5-yl)methoxy)-4-(1-hydroxyethyl)phenyl)acetate (72c)

Compound 72c was prepared according to the procedure reported in step-4 of scheme 1, from ethyl 2-(2-((7-bromobenzofuran-5-yl)methoxy)-4-(1-hydroxyethyl)phenyl)acetate (72b) (1.85 g, 4.27 mmol) in dioxane (50 mL) using 3-(aminomethyl)phenylboronic acid hydrochloride (9e) (0.967 g, 6.40 mmol), bis(triphenylphosphine)palladium(II) chloride (0.450 g, 0.640 mmol), a solution of K₂CO₃ (1.770 g, 12.81 mmol) in water (5 mL) and stirring at 100° C. for 3 h. This gave after workup and purification using flash column chromatography [silica gel (80 g), eluting with MeOH in DCM from 0-10%] followed by [silica gel (24 g), eluting with MeOH in DCM from 0-10%]ethyl 2-(2-((7-(3-(aminomethyl)phenyl)benzofuran-5-yl)methoxy)-4-(1-hydroxyethyl)phenyl)acetate (72c) (1.07 g, 55% yield) as a gummy off white solid; ¹H NMR (300 MHz, DMSO-d₆) δ 8.10 (d, J=2.2 Hz, 1H), 7.85-7.82 (m, 1H), 7.76-7.67 (m, 2H), 7.60 (d, J=1.6 Hz, 1H), 7.47 (t, J=7.6 Hz, 1H), 7.42-7.37 (m, 1H), 7.17-7.11 (m, 2H), 7.05 (d, J=2.2 Hz, 1H), 6.88 (dd, J=7.6, 1.4 Hz, 1H), 5.23 (s, 2H), 4.70 (q, J=6.4 Hz, 1H), 3.91 (q, J=7.1 Hz, 2H), 3.82 (s, 2H), 3.59 (s, 2H), 1.32 (d, J=6.4 Hz, 3H), 0.98 (t, J=7.1 Hz, 3H); MS (ES+): 460.20 (M+1).

### Step-4: Preparation of ethyl 2-(4-acetyl-2-((7-(3-cyanophenyl)benzofuran-5-yl)methoxy)phenyl)acetate (72d)

Compound 72d was prepared according to the procedure reported in step-2 of scheme 25, from ethyl 2-(2-((7-(3-(aminomethyl)phenyl)benzofuran-5-yl)methoxy)-4-(1-hydroxyethyl)phenyl)acetate (72c) (1.05 g, 2.285 mmol) in DCM (20 mL) using Dess-Martin Periodinane (2.040 g, 4.57 mmol) and stirring at RT for 20 h. This gave after workup and purification using flash column chromatography [silica gel (40 g), eluting with ethyl acetate in hexanes from 0-50%]ethyl 2-(4-acetyl-2-((7-(3-cyanophenyl)benzofuran-5-yl)methoxy)phenyl)acetate (72d) (353 mg, 34% yield); MS (ES+): 476.15 (M+Na).

### Step-5: Preparation of ethyl 2-(2-((7-(3-(aminomethyl)phenyl)benzofuran-5-yl)methoxy)-4-(1-hydroxyethyl)phenyl)acetate (72c)

Compound 72c was prepared according to procedure reported in step-2 of scheme 26 from of ethyl 2-(4-acetyl-2-((7-(3-cyanophenyl)benzofuran-5-yl)methoxy)phenyl)acetate (72d) (320 mg, 0.706 mmol) in methanol (25 mL) using nickel(II) chloride hexahydrate (41.9 mg, 0.176 mmol), sodium borohydride (160 mg, 4.23 mmol) and N1-(2-aminoethyl)ethane-1,2-diamine (0.152 mL, 1.411 mmol) for quenching. This gave after workup and purification by flash column chromatography [silica gel (24 g) eluting with MeOH in DCM from 0 to 10%)]ethyl 2-(2-((7-(3-(aminomethyl)phenyl)benzofuran-5-yl)methoxy)-4-(1-hydroxyethyl)phenyl)acetate (72c) (109 mg, 34% yield) as a clear oil; ¹H NMR (300 MHz, DMSO-d₆) δ 8.10 (d, J=2.2 Hz, 1H), 7.86-7.82 (m, 1H), 7.78-7.68 (m, 2H), 7.60 (d, J=1.7 Hz, 1H), 7.48 (t, J=7.6 Hz, 1H), 7.41 (d, J=7.5 Hz, 1H), 7.17-7.11 (m, 2H), 7.05 (d, J=2.2 Hz, 1H), 6.90-6.85 (m, 1H), 5.23 (s, 2H), 4.75-4.63 (m, 1H), 3.91 (q, J=7.1 Hz, 2H), 3.84 (s, 2H), 3.59 (s, 2H), 1.32 (d, J=6.4 Hz, 3H), 0.97 (t, J=7.1 Hz, 3H); MS (ES+): 460.20 (M+1).

### Step-6: Preparation of 2-(2-((7-(3-(aminomethyl)phenyl)benzofuran-5-yl)methoxy)-4-(1-hydroxyethyl)phenyl)acetic acid (72e)

Compound 72e was prepared according to the procedure reported in step-8 of scheme 1, from ethyl 2-(2-((7-(3-(aminomethyl)phenyl)benzofuran-5-yl)methoxy)-4-(1-hydroxyethyl)phenyl)acetate (72c) (95 mg, 0.207 mmol) in THE (5 mL) and methanol (5 mL) using a solution of lithium hydroxide monohydrate (53.1 mg, 1.240 mmol) in water (5 mL) and stirring at RT for 16 h. This gave after workup and purification using reverse phase column chromatography [C18 column (50 g), eluting with ACN in water (containing 0.1% HCl) from 0-70%]2-(2-((7-(3-(aminomethyl)phenyl)benzofuran-5-yl)methoxy)-4-(1-hydroxyethyl)phenyl)acetic acid (72e) (25 mg, 28% yield) HCl salt as a white solid; ¹H NMR (300 MHz, DMSO-d₆) δ 12.21 (s, 1H, D₂O exchangeable), 8.42 (s, 3H, D₂O exchangeable), 8.11 (d, J=2.2 Hz, 1H), 8.04-7.98 (m, 1H), 7.94 (dt, J=7.4, 1.8 Hz, 1H), 7.78 (d, J=1.6 Hz, 1H), 7.67 (d, J=1.6 Hz, 1H), 7.63-7.52 (m, 2H), 7.17-7.10 (m, 2H), 7.07 (d, J=2.2 Hz, 1H), 6.87 (dd, J=7.7, 1.4 Hz, 1H), 5.25 (s, 2H), 5.18 (d, J=4.2 Hz, 1H, D₂O exchangeable), 4.76-4.62 (m, 1H), 4.14 (s, 2H), 3.56 (s, 2H), 1.31 (d, J=6.4 Hz, 3H); MS (ES+): 432.2 (M+1).

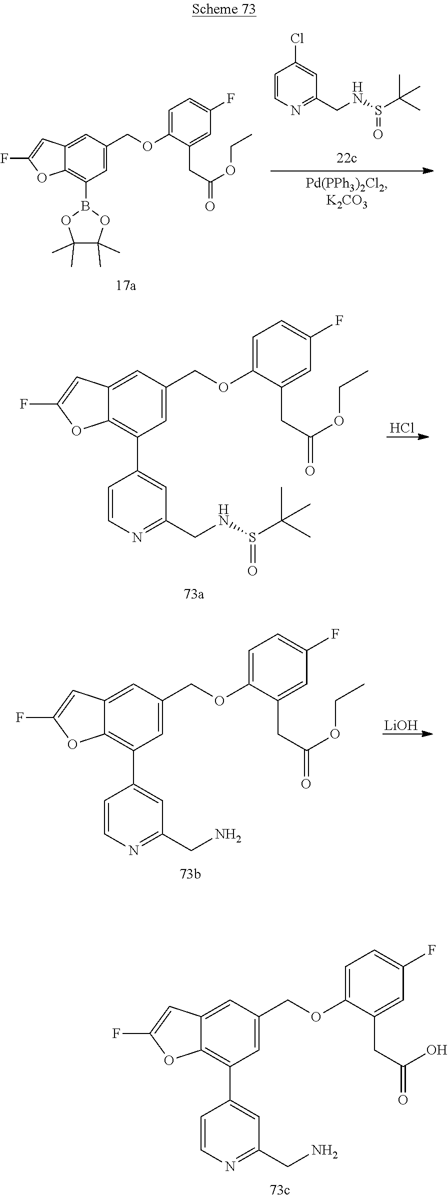

### Preparation of 2-(2-((7-(2-(aminomethyl)pyridin-4-yl)-2-fluorobenzofuran-5-yl)methoxy)-5-fluorophenyl)acetic acid (73c)

### Step-1: Preparation of ethyl 2-(2-((7-(2-((1,1-dimethylethylsulfinamido)methyl)pyridin-4-yl)-2-fluorobenzofuran-5-yl)methoxy)-5-fluorophenyl)acetate (73a)

Compound 73a was prepared according to the procedure reported in step-4 of scheme 1, from ethyl 2-(5-fluoro-2-((2-fluoro-7-(4,4,5,5-tetramethyl-1,3,2-dioxaborolan-2-yl)benzofuran-5-yl)methoxy)phenyl)acetate (17a) (300 mg, 0.635 mmol) in dioxane (12 mL) using (+)-N-((4-chloropyridin-2-yl)methyl)-2-methylpropane-2-sulfinamide (22c) (188 mg, 0.762 mmol), Pd(PPh₃)₂Cl₂ (66.9 mg, 0.095 mmol), K₂CO₃ (263 mg, 1.906 mmol) in water (1.5 mL) and heating under a nitrogen atmosphere at 100° C. for 16 h. This gave after workup and purification by flash column chromatography [silica gel (24 g), eluting with MeOH in DCM from 0-5%](S)-ethyl 2-(2-((7-(2-((1,1-dimethylethylsulfinamido)methyl)pyridin-4-yl)-2-fluorobenzofuran-5-yl)methoxy)-5-fluorophenyl)acetate (73a) (84 mg, 24% yield); MS (ES+): 557.20 (M+1).

### Step-2: Preparation of ethyl 2-(2-((7-(2-(aminomethyl)pyridin-4-yl)-2-fluorobenzofuran-5-yl)methoxy)-5-fluorophenyl)acetate (73b)

Compound 73b was prepared according to procedure reported in step-7 of scheme 1, from ethyl 2-(2-((7-(2-((1,1-dimethylethylsulfinamido)methyl)pyridin-4-yl)-2-fluorobenzofuran-5-yl)methoxy)-5-fluorophenyl)acetate (73a) (75 mg, 0.135 mmol) in THE (10 mL) using 3M aqueous HCl (0.135 mL, 0.404 mmol) and stirring at RT for 2 h. This gave after workup and purification by flash column chromatography [silica gel (12 g), eluting with DMA-80 in DCM from 0-25%]ethyl 2-(2-((7-(2-(aminomethyl)pyridin-4-yl)-2-fluorobenzofuran-5-yl)methoxy)-5-fluorophenyl)acetate (73b) (33 mg, 54% yield); MS (ES+): 453.10 (M+1).

### Step-3: Preparation of 2-(2-((7-(2-(aminomethyl)pyridin-4-yl)-2-fluorobenzofuran-5-yl)methoxy)-5-fluorophenyl)acetic acid (73c)

Compound 73c was prepared according to the procedure reported in step-8 of scheme 1, from ethyl 2-(2-((7-(2-(aminomethyl)pyridin-4-yl)-2-fluorobenzofuran-5-yl)methoxy)-5-fluorophenyl)acetate (73b) (30 mg, 0.066 mmol) in THF (1 mL) and acetonitrile (0.5 mL) using a solution of lithium hydroxide monohydrate (4.76 mg, 0.199 mmol) and stirring at RT for 16 h. This gave after workup and purification using reverse phase column chromatography [C18 column (50 g), eluting with ACN in water (containing 0.1% HCl) from 0-70%]2-(2-((7-(2-(aminomethyl)pyridin-4-yl)-2-fluorobenzofuran-5-yl)methoxy)-5-fluorophenyl)acetic acid (73c) (22 mg, 78% yield) HCl salt as a white solid; ¹H NMR (300 MHz, DMSO-d6) δ 8.78 (d, J=5.3 Hz, 1H), 8.40 (s, 4H, D₂O exchangeable), 7.98 (s, 1H), 7.90 (dd, J=5.2, 1.7 Hz, 1H), 7.79-7.72 (m, 2H), 7.16-7.11 (m, 1H), 7.10-7.05 (m, 2H), 6.50 (d, J=6.4 Hz, 1H), 5.25 (s, 2H), 4.37-4.26 (m, 2H), 3.62 (s, 2H); ¹⁹F NMR (282 MHz, DMSO-d₆) δ −111.11, −123.97; MS (ES+): 425.15 (M+1).

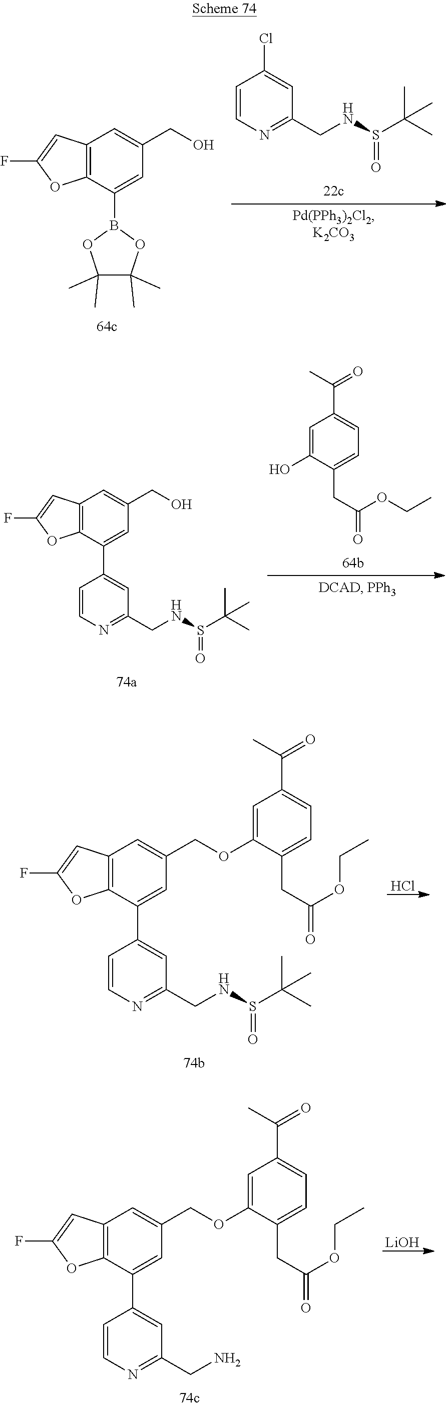

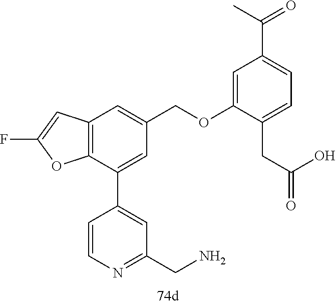 [SMILES: CC(=O)c1ccc(CC(=O)O)c(OCc2cc(-c3ccnc(CN)c3)c3oc(F)cc3c2)c1]

### Preparation of 2-(4-acetyl-2-((7-(2-(aminomethyl)pyridin-4-yl)-2-fluorobenzofuran-5-yl)methoxy)phenyl) acetic acid (74d)

### Step-1: Preparation of N-((4-(2-fluoro-5-(hydroxymethyl)benzofuran-7-yl)pyridin-2-yl)methyl)-2-methylpropane-2-sulfinamide (74a)

Compound 74a was prepared according to the procedure reported in step-4 of scheme 1, from (2-fluoro-7-(4,4,5,5-tetramethyl-1,3,2-dioxaborolan-2-yl)benzofuran-5-yl)methanol (64c) (500 mg, 1.712 mmol) in dioxane (15 mL) using (+)-N-((4-chloropyridin-2-yl)methyl)-2-methylpropane-2-sulfinamide (22c) (422 mg, 1.712 mmol), Pd(PPh₃)₂Cl₂ (180 mg, 0.257 mmol), K₂CO₃ (710 mg, 5.14 mmol) in water (2 mL) and heating under a nitrogen atmosphere at 100° C. for 16 h. This gave after workup and purification by flash column chromatography [silica gel (40 g), eluting with MeOH in DCM from 0-5%]N-((4-(2-fluoro-5-(hydroxymethyl)benzofuran-7-yl)pyridin-2-yl)methyl)-2-methylpropane-2-sulfinamide (74a) (405 mg, 63% yield) as a light brown solid; ¹H NMR (300 MHz, DMSO-d₆) δ 8.64 (d, J=5.2 Hz, 1H), 7.97-7.95 (m, 1H), 7.71 (dd, J=5.1, 1.7 Hz, 1H), 7.63 (d, J=1.6 Hz, 1H), 7.58 (d, J=1.7 Hz, 1H), 6.45 (d, J=6.4 Hz, 1H), 6.01 (t, J=6.2 Hz, 1H), 5.36 (t, J=5.7 Hz, 1H), 4.63 (d, J=5.7 Hz, 2H), 4.46-4.23 (m, 2H), 1.19 (s, 9H); MS (ES+): 377.10 (M+1).

### Step-2: Preparation of ethyl 2-(4-acetyl-2-((7-(2-((1,1-dimethylethylsulfinamido)methyl)pyridin-4-yl)-2-fluorobenzofuran-5-yl)methoxy)phenyl)acetate (74b)

Compound 74b was prepared according to procedure reported in step-2 of scheme 1, from N-((4-(2-fluoro-5-(hydroxymethyl)benzofuran-7-yl)pyridin-2-yl)methyl)-2-methylpropane-2-sulfinamide (74a) (380 mg, 1.009 mmol) in DCM (20 mL) using triphenylphosphine (397 mg, 1.514 mmol), ethyl 2-(4-acetyl-2-hydroxyphenyl)acetate (64b) (224 mg, 1.009 mmol) a solution of (E)-bis(4-chlorobenzyl) diazene-1,2-dicarboxylate (DCAD, 556 mg, 1.514 mmol) in DCM (20 mL) and stirring at room temperature for 2 h. This gave after workup and purification by flash column chromatography [silica gel (40 g), eluting with MeOH in DCM from 0-5%]ethyl 2-(4-acetyl-2-((7-(2-((1,1-dimethylethylsulfinamido)methyl)pyridin-4-yl)-2-fluorobenzofuran-5-yl)methoxy)phenyl)acetate (74b) (489 mg, 83% yield) as a sticky white solid; ¹H NMR (300 MHz, DMSO-d₆) δ 8.66 (d, J=5.2 Hz, 1H), 7.99 (s, 1H), 7.80-7.69 (m, 3H), 7.63-7.54 (m, 2H), 7.40 (d, J=7.7 Hz, 1H), 6.52 (d, J=6.4 Hz, 1H), 5.99 (t, J=6.1 Hz, 1H), 5.33 (s, 2H), 4.50-4.24 (m, 2H), 3.93 (q, J=7.1 Hz, 2H), 3.73 (s, 2H), 2.58 (s, 3H), 1.18 (s, 9H), 0.97 (t, J=7.1 Hz, 3H); MS (ES+): 581.20 (M+1).

### Step-3: Preparation of ethyl 2-(4-acetyl-2-((7-(2-(aminomethyl)pyridin-4-yl)-2-fluorobenzofuran-5-yl)methoxy)phenyl)acetate (74c)

Compound 74c was prepared according to procedure reported in step-7 of scheme 1, from ethyl 2-(4-acetyl-2-((7-(2-((1,1-dimethylethylsulfinamido)methyl)pyridin-4-yl)-2-fluorobenzofuran-5-yl)methoxy)phenyl)acetate (74b) (460 mg, 0.792 mmol) in THE (35 mL) using 3M aqueous HCl (0.792 mL, 2.377 mmol) and stirring at RT for 2 h. Reaction mixture was concentrated to dryness to afford ethyl 2-(4-acetyl-2-((7-(2-(aminomethyl)pyridin-4-yl)-2-fluorobenzofuran-5-yl)methoxy)phenyl)acetate (74c) which was used as such for the next step; MS (ES+): 477.20 (M+1).

### Step-4: Preparation of 2-(4-acetyl-2-((7-(2-(aminomethyl)pyridin-4-yl)-2-fluorobenzofuran-5-yl)methoxy)phenyl)acetic acid (74d)

Compound 74d was prepared according to the procedure reported in step-8 of scheme 1, from ethyl 2-(4-acetyl-2-((7-(2-(aminomethyl)pyridin-4-yl)-2-fluorobenzofuran-5-yl)methoxy)phenyl)acetate (74c) (from step-3 above, 0.792 mmol) in THE (6 mL) and acetonitrile (3 mL) using a solution of lithium hydroxide monohydrate (56.9 mg, 2.376 mmol) in water (3 mL) and stirring at RT for 14 h. This gave after workup and purification using reverse phase column chromatography [C18 column (50 g), eluting with ACN in water (containing 0.1% HCl) from 0-70%]2-(4-acetyl-2-((7-(2-(aminomethyl)pyridin-4-yl)-2-fluorobenzofuran-5-yl)methoxy)phenyl)acetic acid (74d) (109 mg, 31% yield) HCl salt as a white solid; ¹H NMR (300 MHz, DMSO-d₆) δ 8.79 (d, J=5.2 Hz, 1H), 8.47 (s, 3H, D₂O exchangeable), 8.00 (s, 1H), 7.91 (dd, J=5.3, 1.7 Hz, 1H), 7.84-7.78 (m, 2H), 7.63-7.56 (m, 2H), 7.41 (d, J=8.1 Hz, 1H), 6.52 (d, J=6.4 Hz, 1H), 5.37 (s, 2H), 4.38-4.22 (m, 2H), 3.70 (s, 2H), 2.57 (s, 3H); ¹⁹F NMR (282 MHz, DMSO-d₆) δ −111.09; MS (ES+): 449.2 (M+1); Analysis calculated for C₂₅H₂₁FN₂O₅·1.5HCl·2.5H₂O: C, 54.78; H, 5.06; N, 5.11; Cl, 9.70; Found: C, 54.51; H, 4.80; N, 5.11; Cl, 9.86.

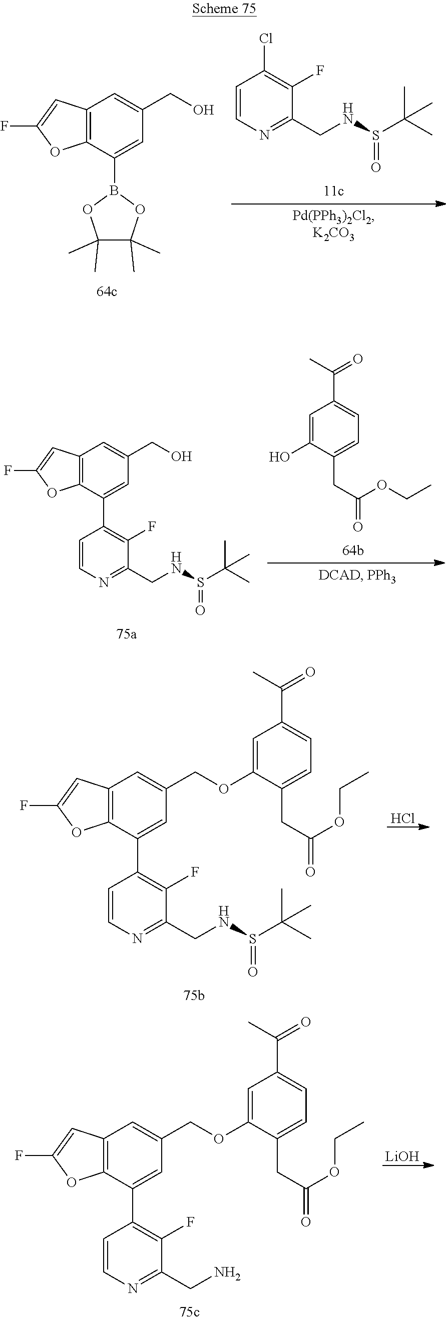

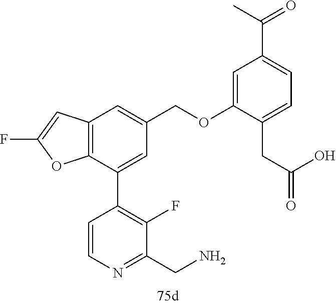 [SMILES: CC(=O)c1ccc(CC(=O)O)c(OCc2cc(-c3ccnc(CN)c3F)c3oc(F)cc3c2)c1]

### Preparation of 2-(4-acetyl-2-((7-(2-(aminomethyl)-3-fluoropyridin-4-yl)-2-fluorobenzofuran-5-yl)methoxy)phenyl)acetic acid (75d)

### Step-1: Preparation of N-((3-fluoro-4-(2-fluoro-5-(hydroxymethyl)benzofuran-7-yl)pyridin-2-yl)methyl)-2-methylpropane-2-sulfinamide (75a)

Compound 75a was prepared according to the procedure reported in step-4 of scheme 1, from (2-fluoro-7-(4,4,5,5-tetramethyl-1,3,2-dioxaborolan-2-yl)benzofuran-5-yl)methanol (64c) (500 mg, 1.712 mmol) in dioxane (15 mL) using (+)-N-((4-chloro-3-fluoropyridin-2-yl)methyl)-2-methylpropane-2-sulfinamide (11c) (453 mg, 1.712 mmol), Pd(PPh₃)₂Cl₂ (180 mg, 0.257 mmol), K₂CO₃ (710 mg, 5.14 mmol) in water (2 mL) and heating under a nitrogen atmosphere at 100° C. for 16 h. This gave after workup and purification by flash column chromatography [silica gel (40 g), eluting with MeOH in DCM from 0-5%]N-((3-fluoro-4-(2-fluoro-5-(hydroxymethyl)benzofuran-7-yl)pyridin-2-yl)methyl)-2-methylpropane-2-sulfinamide (75a) (366 mg, 54% yield); MS (ES+): 395.10 (M+1).

### Step-2: Preparation of ethyl 2-(4-acetyl-2-((7-(2-((1,1-dimethylethylsulfinamido)methyl)-3-fluoropyridin-4-yl)-2-fluorobenzofuran-5-yl)methoxy)phenyl)acetate (75b)

Compound 75b was prepared according to procedure reported in step-2 of scheme 1, from N-((3-fluoro-4-(2-fluoro-5-(hydroxymethyl)benzofuran-7-yl)pyridin-2-yl)methyl)-2-methylpropane-2-sulfinamide (75a) (330 mg, 0.837 mmol) in DCM (20 mL) using triphenylphosphine (329 mg, 1.255 mmol), ethyl 2-(4-acetyl-2-hydroxyphenyl)acetate (64b) (186 mg, 0.837 mmol), a solution of (E)-bis(4-chlorobenzyl) diazene-1,2-dicarboxylate (DCAD, 461 mg, 1.255 mmol) in DCM (20 mL) and stirring at room temperature for 1 h. This gave after workup and purification by flash column chromatography [silica gel (40 g), eluting with EtOAc/MeOH (9:1) in hexanes from 0-50%]ethyl 2-(4-acetyl-2-((7-(2-((1,1-dimethylethylsulfinamido)methyl)-3-fluoropyridin-4-yl)-2-fluorobenzofuran-5-yl)methoxy)phenyl)acetate (75b) (236 mg, 47% yield) as a clear oil; ¹H NMR (300 MHz, DMSO-d₆) δ 8.53 (d, J=4.9 Hz, 1H), 7.81-7.79 (m, 1H), 7.66 (t, J=5.3 Hz, 1H), 7.63-7.53 (m, 3H), 7.40 (d, J=7.6 Hz, 1H), 6.52 (d, J=6.4 Hz, 1H), 5.88 (t, J=5.7 Hz, 1H), 5.33 (s, 2H), 4.45-4.37 (m, 2H), 3.92 (q, J=7.1 Hz, 2H), 3.72 (s, 2H), 2.59 (s, 3H), 1.11 (s, 9H), 0.98 (t, J=7.1 Hz, 3H); MS (ES+): 599.20 (M+1).

### Step-3: Preparation of ethyl 2-(4-acetyl-2-((7-(2-(aminomethyl)-3-fluoropyridin-4-yl)-2-fluorobenzofuran-5-yl)methoxy)phenyl)acetate (75c)

Compound 75c was prepared according to procedure reported in step-7 of scheme 1, from ethyl 2-(4-acetyl-2-((7-(2-((1,1-dimethylethylsulfinamido)methyl)-3-fluoropyridin-4-yl)-2-fluorobenzofuran-5-yl)methoxy)phenyl)acetate (75b) (220 mg, 0.367 mmol) in THE (20 mL) using aqueous HCl, 3M (0.367 mL, 1.102 mmol) and stirring at RT for 2 h. Reaction mixture was concentrated to dryness to afford ethyl 2-(4-acetyl-2-((7-(2-(aminomethyl)-3-fluoropyridin-4-yl)-2-fluorobenzofuran-5-yl)methoxy)phenyl)acetate (75c), which was used as such for the next step; MS (ES+): 495.20 (M+1).

### Step-4: Preparation of 2-(4-acetyl-2-((7-(2-(aminomethyl)-3-fluoropyridin-4-yl)-2-fluorobenzofuran-5-yl)methoxy)phenyl)acetic acid (75d)

Compound 75d was prepared according to the procedure reported in step-8 of scheme 1, from ethyl 2-(4-acetyl-2-((7-(2-(aminomethyl)-3-fluoropyridin-4-yl)-2-fluorobenzofuran-5-yl)methoxy)phenyl)acetate (75c) (from step-3 above, 0.367 mmol) in THE (2 mL) and acetonitrile (1 mL) and water (1 mL) using a solution of lithium hydroxide monohydrate (28.7 mg, 1.2 mmol) and stirring at RT for 3 h. This gave after workup and purification using reverse phase column chromatography [C18 column (50 g), eluting with ACN in water (containing 0.1% HCl) from 0-70%]2-(4-acetyl-2-((7-(2-(aminomethyl)-3-fluoropyridin-4-yl)-2-fluorobenzofuran-5-yl)methoxy)phenyl)acetic acid (75d) (73 mg, 43% yield) HCl salt as a white solid; ¹H NMR (300 MHz, DMSO-d₆) δ 8.65 (d, J=5.0 Hz, 1H), 7.86 (d, J=1.6 Hz, 1H), 7.79 (t, J=5.3 Hz, 1H), 7.65-7.54 (m, 3H), 7.40 (d, J=8.0 Hz, 1H), 6.52 (d, J=6.4 Hz, 1H), 5.36 (s, 2H), 4.38 (s, 2H), 3.68 (s, 2H), 2.58 (s, 3H); ¹⁹F NMR (282 MHz, DMSO-d₆) δ −111.13, −128.50; MS (ES+): 467.1 (M+1); Analysis calculated for C₂₅H₂₀F₂N₂O₅·HCl·1.5H₂O: C, 56.66; H, 4.56; N, 5.29; Cl, 6.69; Found: C, 56.52; H, 4.60; N, 5.22; Cl, 6.39.

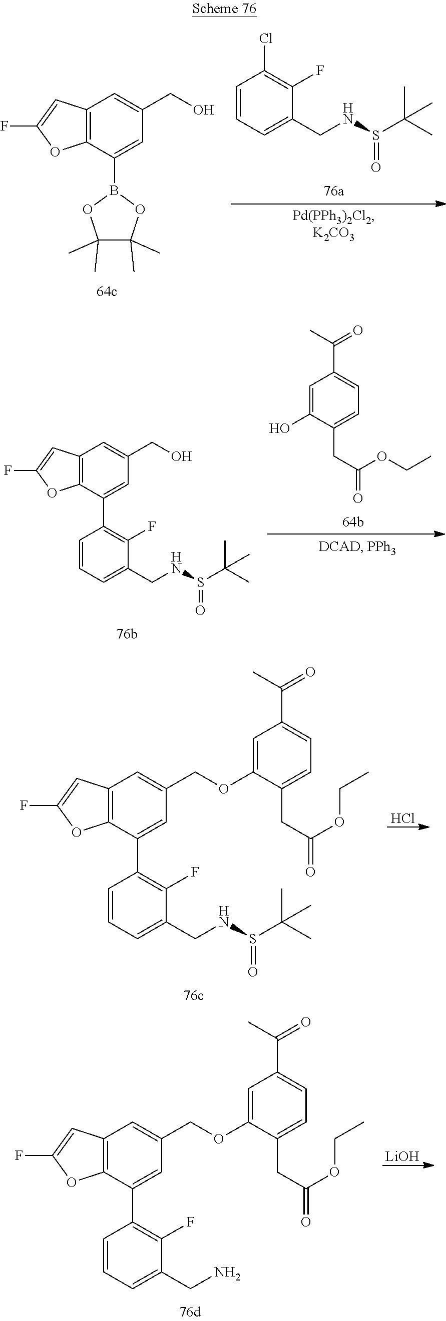

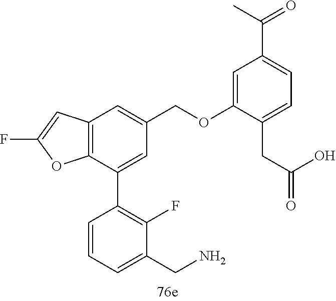 [SMILES: CC(=O)c1ccc(CC(=O)O)c(OCc2cc(-c3cccc(CN)c3F)c3oc(F)cc3c2)c1]

### Preparation of 2-(4-acetyl-2-((7-(3-(aminomethyl)-2-fluorophenyl)-2-fluorobenzofuran-5-yl)methoxy)phenyl)acetic acid (76e)

### Step-1: Preparation of N-(2-fluoro-3-(2-fluoro-5-(hydroxymethyl)benzofuran-7-yl)benzyl)-2-methylpropane-2-sulfinamide (76b)

Compound 76b was prepared according to the procedure reported in step-4 of scheme 1, from (2-fluoro-7-(4,4,5,5-tetramethyl-1,3,2-dioxaborolan-2-yl)benzofuran-5-yl)methanol (64c) (1.0 g, 3.42 mmol) in dioxane (30 mL) using (+)-N-(3-chloro-2-fluorobenzyl)-2-methylpropane-2-sulfinamide (76a) (0.903 g, 3.42 mmol), Pd(PPh₃)₂Cl₂ (0.360 g, 0.514 mmol), K₂CO₃ (1.419 g, 10.27 mmol) in water (4 mL) and heating under a nitrogen atmosphere at 100° C. for 16 h. This gave after workup and purification by flash column chromatography [silica gel (120 g), eluting with ethyl acetate in hexanes from 0-50%]N-(2-fluoro-3-(2-fluoro-5-(hydroxymethyl)benzofuran-7-yl)benzyl)-2-methylpropane-2-sulfinamide (76b) (40 mg, 3% yield); ¹H NMR (300 MHz, DMSO-d₆) δ 7.61-7.45 (m, 3H), 7.37-7.26 (m, 2H), 6.40 (d, J=6.5 Hz, 1H), 5.87 (t, J=6.0 Hz, 1H), 5.32 (t, J=5.8 Hz, 1H), 4.61 (d, J=5.7 Hz, 2H), 4.40-4.15 (m, 2H), 1.14 (s, 9H); MS (ES+): 394.10 (M+1).

### Step-2: Preparation of ethyl 2-(4-acetyl-2-((7-(3-((1,1-dimethylethylsulfinamido)methyl)-2-fluorophenyl)-2-fluorobenzofuran-5-yl)methoxy)phenyl)acetate (76c)

Compound 76c was prepared according to procedure reported in step-2 of scheme 1, from N-(2-fluoro-3-(2-fluoro-5-(hydroxymethyl)benzofuran-7-yl)benzyl)-2-methylpropane-2-sulfinamide (76b) (35 mg, 0.089 mmol) in DCM (10 mL) using triphenylphosphine (35.0 mg, 0.133 mmol), ethyl 2-(4-acetyl-2-hydroxyphenyl)acetate (64b) (19.77 mg, 0.089 mmol), a solution of (E)-bis(4-chlorobenzyl) diazene-1,2-dicarboxylate (DCAD, 49.0 mg, 0.133 mmol) in DCM (10 mL) and stirring at room temperature for 16 h. This gave after workup and purification by flash column chromatography [silica gel (40 g), eluting with 5% MeOH in DCM]ethyl 2-(4-acetyl-2-((7-(3-((1,1-dimethylethylsulfinamido)methyl)-2-fluorophenyl)-2-fluorobenzofuran-5-yl)methoxy)phenyl)acetate (76c) (12 mg, 23% yield); MS (ES+): 598.20 (M+1).

### Step-3: Preparation of ethyl 2-(4-acetyl-2-((7-(3-(aminomethyl)-2-fluorophenyl)-2-fluorobenzofuran-5-yl)methoxy)phenyl)acetate (76d)

Compound 76d was prepared according to procedure reported in step-7 of scheme 1, from ethyl 2-(4-acetyl-2-((7-(3-((1,1-dimethylethylsulfinamido)methyl)-2-fluorophenyl)-2-fluorobenzofuran-5-yl)methoxy)phenyl)acetate (76c) (10 mg, 0.017 mmol) in THE (5 mL) using 3M aqueous HCl (0.017 mL, 0.05 mmol) and stirring at RT for 2 h. This gave after workup ethyl 2-(4-acetyl-2-((7-(3-(aminomethyl)-2-fluorophenyl)-2-fluorobenzofuran-5-yl)methoxy)phenyl)acetate (76d), which was used as such for the next step; MS (ES+): 494.20 (M+1).

### Step-4: Preparation of 2-(4-acetyl-2-((7-(3-(aminomethyl)-2-fluorophenyl)-2-fluorobenzofuran-5-yl)methoxy)phenyl)acetic acid (76e)

Compound 76e was prepared according to the procedure reported in step-8 of scheme 1, from ethyl 2-(4-acetyl-2-((7-(3-(aminomethyl)-2-fluorophenyl)-2-fluorobenzofuran-5-yl)methoxy)phenyl)acetate (76d) (from step-3 above, 0.017 mmol) in THE (1 mL) and acetonitrile (0.5 mL) using a solution of lithium hydroxide monohydrate (1.221 mg, 0.051 mmol) in water (0.5 mL) and stirring at RT for 14 h. This gave after workup and purification using reverse phase column chromatography [C18 column (50 g), eluting with ACN in water (containing 0.1% HCl) from 0-70%]2-(4-acetyl-2-((7-(3-(aminomethyl)-2-fluorophenyl)-2-fluorobenzofuran-5-yl)methoxy)phenyl)acetic acid (76e) (2.8 mg, 35% yield) HCl salt as a white solid; ¹H NMR (300 MHz, DMSO-d₆) δ 7.77 (d, J=1.6 Hz, 1H), 7.72-7.63 (m, 2H), 7.62-7.55 (m, 2H), 7.52-7.47 (m, 1H), 7.46-7.35 (m, 2H), 6.46 (d, J=6.5 Hz, 1H), 5.34 (s, 2H), 4.19 (s, 2H), 3.67 (s, 2H), 2.57 (s, 3H); ¹⁹F NMR (282 MHz, DMSO-d₆) δ −111.53, −118.43; MS (ES+): 466.15 (M+1).

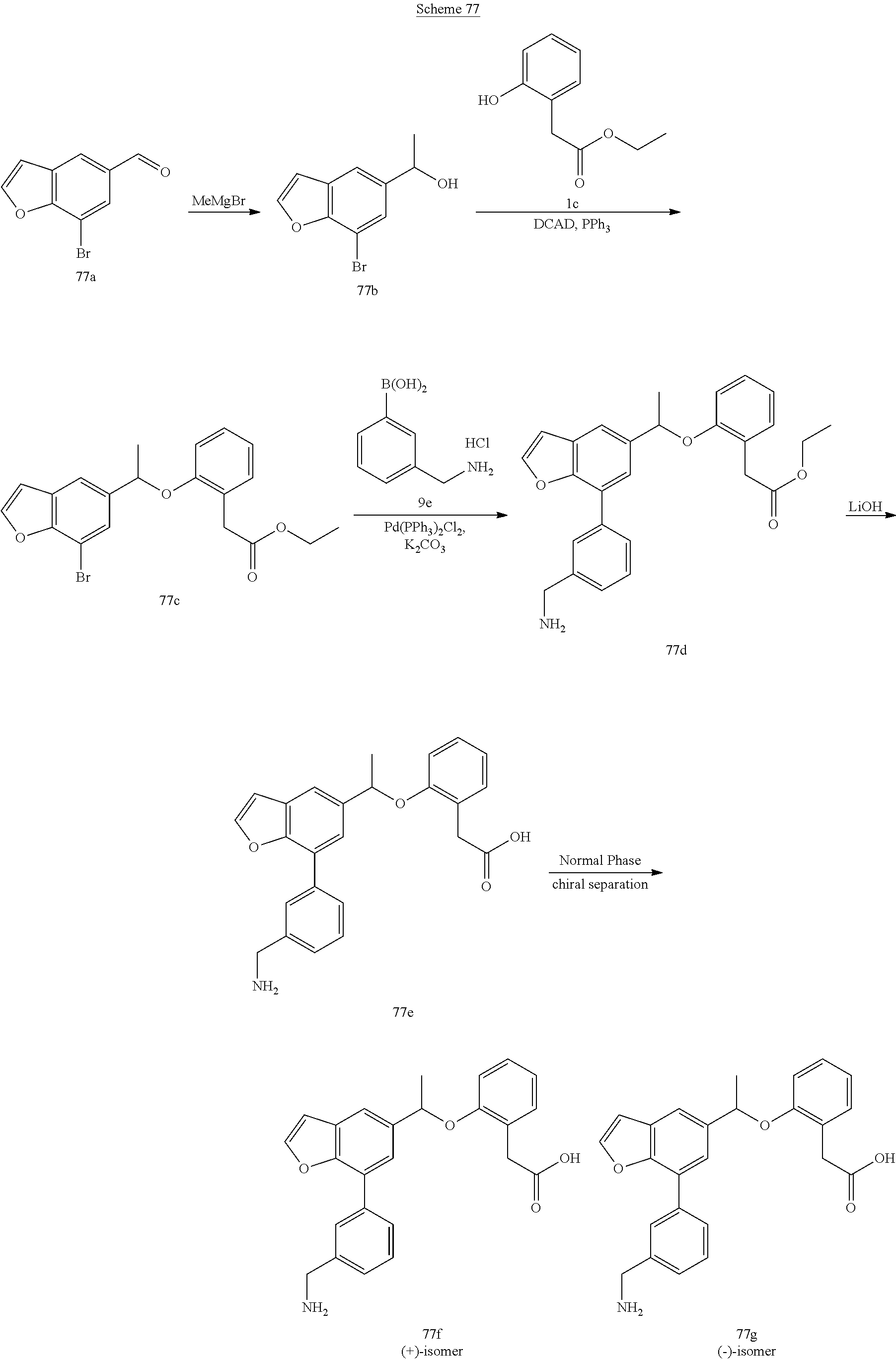

### Preparation of (+)-2-(2-(1-(7-(3-(aminomethyl)phenyl)benzofuran-5-yl)ethoxy)phenyl)acetic acid (77f) and (−)-2-(2-(1-(7-(3-(aminomethyl)phenyl)benzofuran-5-yl)ethoxy)phenyl)acetic acid (77g)

### Step-1: Preparation of 1-(7-bromobenzofuran-5-yl)ethanol (77b)

Compound 77b was prepared according to procedure reported in step-6 of scheme 1, from 7-bromobenzofuran-5-carbaldehyde (77a) (800 mg, 3.55 mmol) in THE (20 mL) using methyl magnesium bromide (1.4 M in THF) (2.79 mL, 3.91 mmol) and stirring at −78° C. for 1 h. This gave after workup and purification by flash column chromatography [silica gel (40 g), eluting with EtOAc in hexane]1-(7-bromobenzofuran-5-yl)ethanol (77b) (630 mg, 74% yield) as a white oil; 1H NMR (300 MHz, DMSO-d₆) δ 8.09 (d, J=2.2 Hz, 1H), 7.61 (d, J=1.5 Hz, 1H), 7.52 (d, J=1.5 Hz, 1H), 7.06 (d, J=2.2 Hz, 1H), 5.31 (d, J=4.2 Hz, 1H), 4.82 (qd, J=6.4, 4.2 Hz, 1H), 1.35 (d, J=6.4 Hz, 3H).

### Step-2: Preparation of ethyl 2-(2-(1-(7-bromobenzofuran-5-yl)ethoxy)phenyl)acetate (77c)

Compound 77c was prepared according to procedure reported in step-7 of scheme 25, from 1-(7-bromobenzofuran-5-yl)ethanol (77b) (630 mg, 2.61 mmol) in DCM (20 mL) using triphenylphosphine (720 mg, 2.74 mmol), ethyl 2-(2-hydroxyphenyl)acetate (1c) (494 mg, 2.74 mmol), a solution of (E)-bis(4-chlorobenzyl) diazene-1,2-dicarboxylate (DCAD, 1008 mg, 2.74 mmol) in DCM (10 mL) and stirring at room temperature for 1 h. This gave after workup and purification by flash column chromatography [silica gel (24 g), eluting with DMA-80 in DCM from 0-60%]ethyl 2-(2-(1-(7-bromobenzofuran-5-yl)ethoxy)phenyl)acetate (77c) (500 mg, 47% yield); 1H NMR (300 MHz, DMSO-d₆) δ 8.11 (d, J=2.2 Hz, 1H), 7.68 (d, J=1.5 Hz, 1H), 7.58 (d, J=1.5 Hz, 1H), 7.19 (dd, J=7.7, 1.7 Hz, 1H), 7.11 (dd, J=8.1, 1.8 Hz, 1H), 7.06 (d, J=2.3 Hz, 1H), 6.82 (dd, J=7.5, 6.4 Hz, 2H), 5.61 (q, J=6.3 Hz, 1H), 4.15-4.06 (m, 2H), 3.67 (s, 2H), 1.52 (d, J=6.3 Hz, 3H), 1.20 (t, J=7.2 Hz, 3H).

### Step-3: Preparation of ethyl 2-(2-(1-(7-(3-(aminomethyl)phenyl)benzofuran-5-yl)ethoxy)phenyl)acetate (77d)

Compound 77d was prepared according to the procedure reported in step-4 of scheme 1, from ethyl 2-(2-(1-(7-bromobenzofuran-5-yl)ethoxy)phenyl)acetate (77c) (250 mg, 0.620 mmol) in dioxane (5 mL) using 3-(aminomethyl)phenylboronic acid hydrochloride (9e) (0.232 g, 1.240 mmol), Pd(PPh₃)₂Cl₂ (65.3 mg, 0.093 mmol), K₂CO₃ (257 mg, 1.860 mmol) in water (1 mL) and heating under a nitrogen atmosphere at 90° C. for 3 h. This gave after workup and purification by flash column chromatography [silica gel (12 g), eluting with DMA-80 in DCM from 0-50%]ethyl 2-(2-(1-(7-(3-(aminomethyl)phenyl)benzofuran-5-yl)ethoxy)phenyl)acetate (77d) (150 mg, 56% yield); 1H NMR (300 MHz, DMSO-d₆) δ 8.07 (d, J=2.2 Hz, 1H), 7.81 (d, J=1.9 Hz, 1H), 7.71 (dt, J=7.5, 1.6 Hz, 1H), 7.66 (d, J=1.7 Hz, 1H), 7.56 (d, J=1.7 Hz, 1H), 7.46 (t, J=7.5 Hz, 1H), 7.39 (d, J=7.5 Hz, 1H), 7.19 (dd, J=7.4, 1.7 Hz, 1H), 7.10 (td, J=7.8, 1.8 Hz, 1H), 7.00 (d, J=2.2 Hz, 1H), 6.88 (d, J=8.2 Hz, 1H), 6.84-6.76 (m, 1H), 5.66 (q, J=6.2 Hz, 1H), 4.16-4.02 (m, 2H), 3.82 (s, 2H), 3.69 (s, 2H), 1.60 (d, J=6.3 Hz, 3H), 1.17 (t, J=7.1 Hz, 3H). MS (ES+): 430.20 (M+1); MS (ES−): 428.20 (M−1).

### Step-4: Preparation of 2-(2-(1-(7-(3-(aminomethyl)phenyl)benzofuran-5-yl)ethoxy)phenyl)acetic acid (77e)

Compound 77e was prepared according to the procedure reported in step-8 of scheme 1, from ethyl 2-(2-(1-(7-(3-(aminomethyl)phenyl)benzofuran-5-yl)ethoxy)phenyl)acetate (77d) (150 mg, 0.349 mmol) in THE (2 mL) and MeOH (2 mL) using a solution of lithium hydroxide hydrate (88 mg, 2.095 mmol) in water (1 mL) and stirring at RT for 15 h. This gave after workup and purification using reverse phase column chromatography [C18 column (50 g), eluting with ACN in water (containing 0.1% HCl) from 0-100%]2-(2-(1-(7-(3-(aminomethyl)phenyl)benzofuran-5-yl)ethoxy)phenyl)acetic acid (77e) (85 mg, 61% yield) HCl salt as a white solid; ¹H NMR (300 MHz, DMSO-d₆) δ 12.27 (s, 1H, D₂O exchangeable), 8.53 (s, 3H, D₂O exchangeable), 8.07 (d, J=2.2 Hz, 1H), 8.01 (d, J=2.3 Hz, 1H), 7.91 (m, 1H), 7.71 (d, J=1.6 Hz, 1H), 7.64 (d, J=1.7 Hz, 1H), 7.59-7.54 (m, 2H), 7.18 (dd, J=7.4, 1.7 Hz, 1H), 7.07 (td, J=7.8, 1.8 Hz, 1H), 7.00 (d, J=2.2 Hz, 1H), 6.85 (d, J=8.2 Hz, 1H), 6.79 (td, J=7.4, 1.0 Hz, 1H), 5.65 (q, J=6.2 Hz, 1H), 4.12 (s, 2H), 3.64 (s, 2H), 1.61 (d, J=6.2 Hz, 3H); MS (ES+): 402.15 (M+1); (ES−): 400.15 (M−1).

### Step-5: Preparation of (+)-2-(2-(1-(7-(3-(aminomethyl)phenyl)benzofuran-5-yl)ethoxy)phenyl)acetic acid (77f) and (−)-2-(2-(1-(7-(3-(aminomethyl)phenyl)benzofuran-5-yl)ethoxy)phenyl)acetic acid (77g)

Compound 2-(2-(1-(7-(3-(aminomethyl)phenyl)benzofuran-5-yl)ethoxy)phenyl)acetic acid (77e) (2.5 gms) was subjected to Preparative normal phase chiral separation using the following method.

### Chiral Preparative Method:

Column: Chiralpak IA 250×30 mm, 5 micron; Mobile Phase A: 0.1% Isopropyl amine in Hexane/MTBE (50:50); Mobilephase B: DCM:Methanol (50:50); Isocratic A:B: 80:20; Flowrate: 25 mL/min; Diluent: Isopropylamine+DCM+Methanol (50:25:25).

### Chiral Analytical Method:

Column: DIACEL CHIRALPAK-IA, 250 mm*4.6 mm, 5u Mobile Phase: A: n-HEXANE+MTBE (50:50)+0.1% IPA B: DCM:MeOH (50:50); Flow rate: 1.0 mL/min; Isocratic: 20% B; Injection Volume: 10 ul; Detector PDA 271 nm 4 nm.

This gave the following:

Isomer I as (−)-2-(2-(1-(7-(3-(aminomethyl)phenyl)benzofuran-5-yl)ethoxy)phenyl)acetic acid (77g) [Retention time=9.567 min (chiral analytical method), 985 mg, 39% isolated yield, 98.59% chiral purity, 99.16 achiral purity, 97.18% ee {isomer-1 retention time 9.567 (98.59%); isomer-2 retention time 14.920 (1.410%)}]; ¹H NMR (300 MHz, DMSO-d₆) δ 8.20 (s, 1H), 8.10-7.98 (m, 2H), 7.81-7.68 (m, 2H), 7.50-7.38 (m, 1H), 7.37-7.25 (m, 1H), 7.14-7.06 (m, 1H), 7.06-7.00 (m, 1H), 6.99-6.85 (m, 1H), 6.84-6.76 (m, 1H), 6.75-6.61 (m, 1H), 5.77-5.62 (m, 1H), 3.97 (s, 2H), 3.80 (d, J=14.6 Hz, 1H), 3.08 (d, J=14.4 Hz, 1H), 1.58 (d, J=5.4 Hz, 3H); MS (ES+): 402.40 (M+1); (ES−): 399.95 (M−1); Optical rotation [α]_{D}=−75.77 (c=0.5, DMF); 330 mgs of this Isomer I was converted to HCl salt using reverse phase column chromatography [C18 column (50 g), eluting with ACN in water (containing 0.1% HCl) from 0-100%] to furnish 260 mgs (−)-2-(2-(1-(7-(3-(aminomethyl)phenyl)benzofuran-5-yl)ethoxy)phenyl)acetic acid (77g) HCl salt as a white solid; ¹H NMR (300 MHz, DMSO-d₆) δ 12.25 (s, 1H, D₂O exchangeable), 8.27 (s, 3H, D₂O exchangeable), 8.08 (d, J=2.2 Hz, 1H), 7.99-7.94 (m, 1H), 7.90 (dt, J=7.6, 1.5 Hz, 1H), 7.72 (d, J=1.6 Hz, 1H), 7.62-7.59 (m, 1H), 7.57 (d, J=7.6 Hz, 1H), 7.55-7.49 (m, 1H), 7.18 (dd, J=7.4, 1.7 Hz, 1H), 7.11-7.03 (m, 1H), 7.01 (d, J=2.2 Hz, 1H), 6.87-6.74 (m, 2H), 5.65 (q, J=6.1 Hz, 1H), 4.14 (s, 2H), 3.63 (s, 2H), 1.61 (d, J=6.3 Hz, 3H); MS (ES+): 402.10 (M+1); (ES−): 400.10 (M−1); Optical rotation [α]_{D}=−70.00 (c=0.3, 1:1 H₂O/MeOH); Analysis calculated for C₂₅H₂₃NO₄·HCl·5H₂O: C, 67.19; H, 5.64; Cl, 7.93; N, 3.13; Found: C, 67.22; H, 5.75; Cl, 8.02; N, 3.21.

Isomer II as (+)-2-(2-(1-(7-(3-(aminomethyl)phenyl)benzofuran-5-yl)ethoxy)phenyl)acetic acid (77f) [Retention time=14.471 min (chiral analytical method), 830 mg, 33% isolated yield, 98.40% chiral purity, 97.69 achiral purity, 96.82% ee {isomer-1 retention time 10.128 (1.592%); isomer-2 retention time 14.392 (98.408%)}]; ¹H NMR (300 MHz, DMSO-d₆) δ 8.22 (s, 1H), 8.12-7.98 (m, 2H), 7.81-7.69 (m, 2H), 7.49-7.38 (m, 1H), 7.38-7.26 (m, 1H), 7.15-6.98 (m, 2H), 6.98-6.86 (m, 1H), 6.85-6.75 (m, 1H), 6.75-6.63 (m, 1H), 5.79-5.59 (m, 1H), 3.98 (s, 2H), 3.84-3.73 (m, 1H), 3.12-3.01 (m, 1H), 1.64-1.52 (m, 3H); MS (ES+): 402.50 (M+1); (ES−): 399.95 (M−1); Optical rotation [α]_{D}=+77.03 (c=0.5, DMF); 270 mgs of Isomer II was converted to HCl salt using reverse phase column chromatography [C18 column (50 g), eluting with ACN in water (containing 0.1% HCl) from 0-100%] to furnish 220 mgs of (+)-2-(2-(1-(7-(3-(aminomethyl)phenyl)benzofuran-5-yl)ethoxy)phenyl)acetic acid (77f) HCl salt as a white solid; ¹H NMR (300 MHz, DMSO-d₆) δ 12.25 (s, 1H, D₂O exchangeable), 8.28 (s, 3H, D₂O exchangeable), 8.08 (d, J=2.2 Hz, 1H), 7.97 (s, 1H), 7.90 (dt, J=7.6, 1.6 Hz, 1H), 7.72 (d, J=1.6 Hz, 1H), 7.60 (d, J=2.4 Hz, 1H), 7.57 (d, J=7.5 Hz, 1H), 7.55-7.49 (m, 1H), 7.18 (dd, J=7.4, 1.8 Hz, 1H), 7.07 (td, J=7.9, 7.3, 1.8 Hz, 1H), 7.01 (d, J=2.2 Hz, 1H), 6.87-6.75 (m, 2H), 5.65 (q, J=6.3 Hz, 1H), 4.14 (s, 2H), 3.63 (s, 2H), 1.61 (d, J=6.3 Hz, 3H); MS (ES+): MS (ES+): 402.10 (M+1); (ES−): 400.10 (M−1); Optical rotation [α]_{D}=+67.08 (c=0.325, 1:1 H₂O/MeOH); Analysis calculated for C₂₅H₂₃NO₄·HCl·5H₂O: C, 67.19; H, 5.64; Cl, 7.93; N, 3.13; Found: C, 67.12; H, 5.81; Cl, 8.01; N, 3.24.

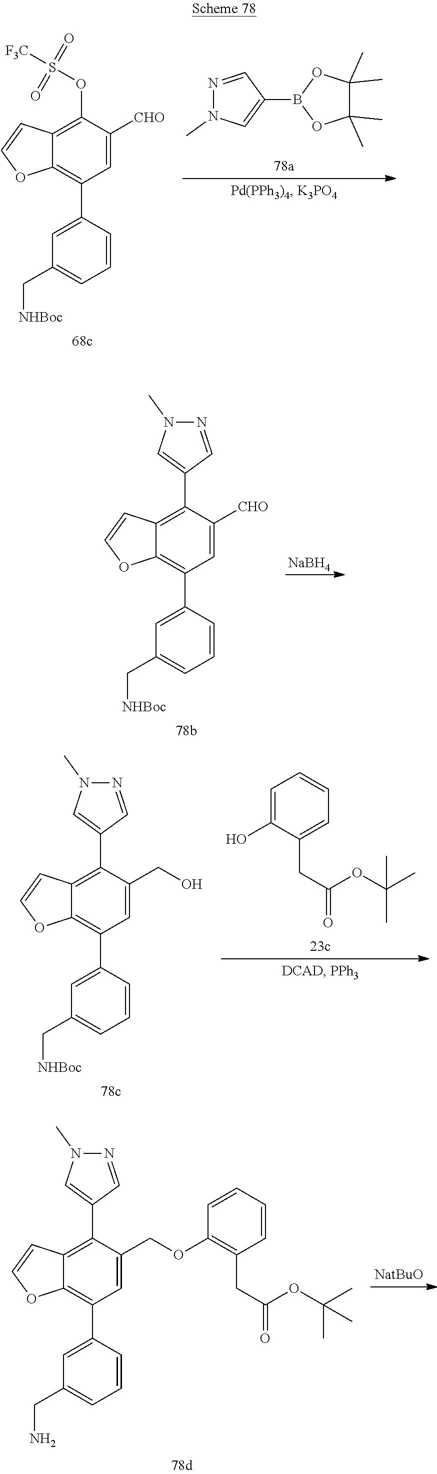

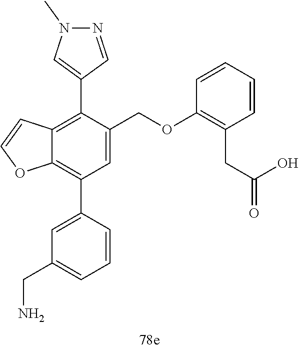 [SMILES: Cn1cc(-c2c(COc3ccccc3CC(=O)O)cc(-c3cccc(CN)c3)c3occc23)cn1]

### Preparation of 2-(2-((7-(3-(aminomethyl)phenyl)-4-(1-methyl-1H-pyrazol-4-yl)benzofuran-5-yl)methoxy)phenyl)acetic acid (78e)

### Step-1: Preparation of tert-butyl 3-(5-formyl-4-(1-methyl-1H-pyrazol-4-yl)benzofuran-7-yl)benzylcarbamate (78b)

Compound 78b was prepared according to the procedure reported in step-4 of scheme 1, from 7-(3-(((tert-butoxycarbonyl)amino)methyl)phenyl)-5-formylbenzofuran-4-yl trifluoromethanesulfonate (68c) (190 mg, 0.380 mmol) in dioxane (6 mL) using 1-methyl-4-(4,4,5,5-tetramethyl-1,3,2-dioxaborolan-2-yl)-1H-pyrazole (78a) (158 mg, 0.761 mmol; CAS #761446-44-0), Pd(PPh₃)₄ (44 mg, 0.038 mmol), potassium phosphate (161 mg, 0.761 mmol) and stirring at 85° C. for 170 min. This gave after work up and purification [silica gel (12 g), eluting with EtOAc/MeOH (9:1) in hexane from 0-100%] tert-butyl 3-(5-formyl-4-(1-methyl-1H-pyrazol-4-yl)benzofuran-7-yl)benzylcarbamate (78b) (142 mg, 87% yield) as a yellow solid; ¹H NMR (300 MHz, DMSO-d₆) δ 10.20 (s, 1H), 8.27-8.19 (m, 2H), 8.01 (s, 1H), 7.84 (s, 1H), 7.82-7.73 (m, 2H), 7.56-7.47 (m, 2H), 7.34 (d, J=7.7 Hz, 1H), 7.19 (d, J=2.3 Hz, 1H), 4.25 (d, J=6.2 Hz, 2H), 3.99 (s, 3H), 1.41 (s, 9H); MS (ES+): 432.20 (M+1).

### Step-2: Preparation of tert-butyl 3-(5-(hydroxymethyl)-4-(1-methyl-1H-pyrazol-4-yl)benzofuran-7-yl)benzylcarbamate (78c)

Compound 78c was prepared according to the procedure reported in step-3 of scheme 5, from tert-butyl 3-(5-formyl-4-(1-methyl-1H-pyrazol-4-yl)benzofuran-7-yl)benzylcarbamate (78b) (142 mg, 0.329 mmol) in THE (2.5 mL) and MeOH (2.5 mL) using sodium borohydride (37.4 mg, 0.987 mmol) and stirring at RT for 5 min. This gave after workup and purification using flash column chromatography [silica gel (12 g), eluting with EtOAc/MeOH (9:1) in hexane from 0-100%] tert-butyl 3-(5-(hydroxymethyl)-4-(1-methyl-1H-pyrazol-4-yl)benzofuran-7-yl)benzylcarbamate (78c) (130 mg, 91% yield) as a colorless foam; H NMR (300 MHz, DMSO-d₆) δ 8.10-8.04 (m, 2H), 7.81-7.72 (m, 3H), 7.66 (s, 1H), 7.55-7.44 (m, 2H), 7.29 (d, J=7.4 Hz, 1H), 7.01 (d, J=2.3 Hz, 1H), 5.26 (t, J=5.3 Hz, 1H), 4.60 (d, J=5.3 Hz, 2H), 4.24 (d, J=6.3 Hz, 2H), 3.96 (s, 3H), 1.41 (s, 9H).

### Step-3: Preparation of tert-butyl 2-(2-((7-(3-(((tert-butoxycarbonyl)amino)methyl)phenyl)-4-(1-methyl-1H-pyrazol-4-yl)benzofuran-5-yl)methoxy)phenyl)acetate (78d)

Compound 78d was prepared according to procedure reported in step-2 of scheme 1, from tert-butyl 3-(5-(hydroxymethyl)-4-(1-methyl-1H-pyrazol-4-yl)benzofuran-7-yl)benzylcarbamate (78c) (95 mg, 0.219 mmol) in DCM (8 mL) using tert-butyl 2-(2-hydroxyphenyl)acetate (23c) (91 mg, 0.438 mmol), triphenylphosphine (115 mg, 0.438 mmol), a solution of (E)-bis(4-chlorobenzyl) diazene-1,2-dicarboxylate (DCAD, 161 mg, 0.438 mmol) in DCM (2 mL) and stirring at room temperature for 2 h. This gave after workup and purification using flash column chromatography [silica gel (12 g), eluting with EtOAc/MeOH (9:1) in hexane from 0-100%] tert-butyl 2-(2-((7-(3-(((tert-butoxycarbonyl)amino)methyl)phenyl)-4-(1-methyl-1H-pyrazol-4-yl)benzofuran-5-yl)methoxy)phenyl)acetate (78d) (90 mg, 66% yield) as a clear oil; MS (ES+): 646.2 (M+Na).

### Step-4: Preparation of 2-(2-((7-(3-(aminomethyl)phenyl)-4-(1-methyl-1H-pyrazol-4-yl)benzofuran-5-yl)methoxy)phenyl)acetic acid (78e)

Compound 78e was prepared according to procedure reported in step-6 of scheme 68, from tert-butyl 2-(2-((7-(3-(((tert-butoxycarbonyl)amino)methyl)phenyl)-4-(1-methyl-1H-pyrazol-4-yl)benzofuran-5-yl)methoxy)phenyl)acetate (78d) (90 mg, 0.144 mmol) in THE (4 mL) using sodium tert-butoxide (100 mg, 1.041 mmol) and water (10.00 μL, 0.555 mmol) and heating to 67° C. for 5 days. This gave after workup and purification using flash column chromatography [C18 column (50 g), eluting with ACN in water from 0-100%]2-(2-((7-(3-(aminomethyl)phenyl)-4-(1-methyl-1H-pyrazol-4-yl)benzofuran-5-yl)methoxy)phenyl)acetic acid (78e) (21 mg, 31% yield) as a white solid; ¹H NMR (300 MHz, DMSO-d₆) δ 8.31 (s, 1H), 8.13 (d, J=9.0 Hz, 3H), 8.06 (s, 1H), 7.81 (s, 1H), 7.48 (t, J=7.7 Hz, 1H), 7.34 (d, J=7.7 Hz, 1H), 7.17-6.94 (m, 3H), 6.80 (t, J=7.3 Hz, 1H), 6.72 (d, J=8.2 Hz, 1H), 5.29 (s, 2H), 3.99 (s, 5H), 3.39 (s, 2H); MS (ES+): 468.2 (M+1); (ES−): 466.2 (M−1).

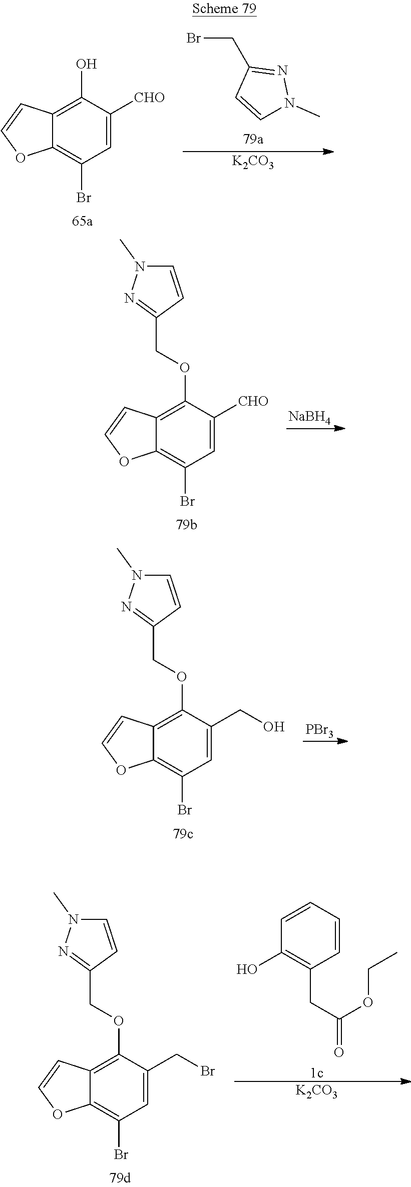

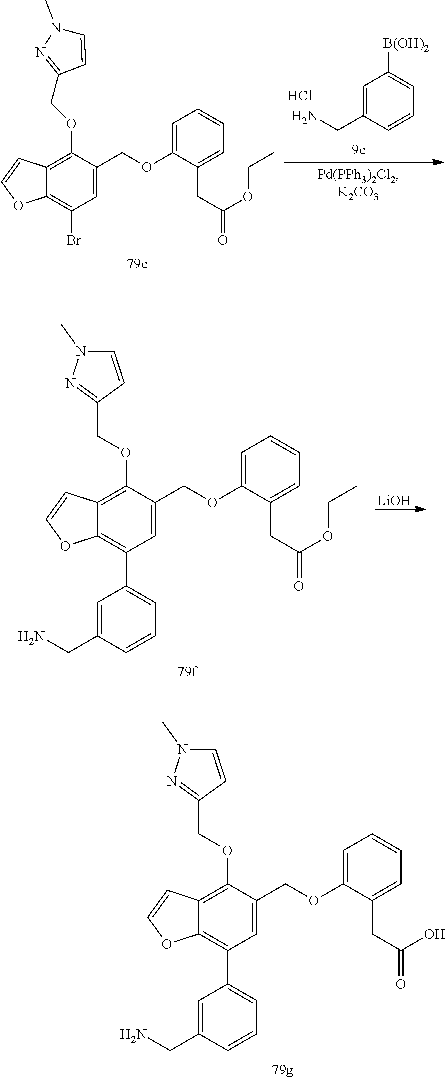

### Preparation of 2-(2-((7-(3-(aminomethyl)phenyl)-4-((1-methyl-1H-pyrazol-3-yl)methoxy)benzofuran-5-yl)methoxy)phenyl)acetic acid (79g)

### Step-1: Preparation of 7-bromo-4-((1-methyl-1H-pyrazol-3-yl)methoxy)benzofuran-5-carbaldehyde (79b)

Compound 79b was prepared according to the procedure reported in step-4 of scheme 14, from 7-bromo-4-hydroxybenzofuran-5-carbaldehyde (65a) (256 mg, 1.062 mmol) in acetone (6 mL) using 3-(bromomethyl)-1-methyl-1H-pyrazole (79a) (360 mg, 2.057 mmol; CAS #102846-13-9), K₂CO₃ (440 mg, 3.19 mmol) and stirring overnight at RT. This gave after workup and purification using flash column chromatography [silica gel (12 g), eluting with EtOAc/MeOH (9:1) in hexane from 0-80%]7-bromo-4-((1-methyl-1H-pyrazol-3-yl)methoxy)benzofuran-5-carbaldehyde (79b) (333 mg, 94% yield) as a white solid; ¹H NMR (300 MHz, DMSO-d₆) δ 10.15 (s, 1H), 8.26 (d, J=2.3 Hz, 1H), 7.76 (s, 1H), 7.67 (d, J=2.2 Hz, 1H), 7.58 (d, J=2.3 Hz, 1H), 6.33 (d, J=2.2 Hz, 1H), 5.44 (s, 2H), 3.80 (s, 3H).

### Step-2: Preparation of (7-bromo-4-((1-methyl-1H-pyrazol-3-yl)methoxy)benzofuran-5-yl)methanol (79c)

Compound 79c was prepared according to the procedure reported in step-3 of scheme 5, from 7-bromo-4-((1-methyl-1H-pyrazol-3-yl)methoxy)benzofuran-5-carbaldehyde (79b) (165 mg, 0.492 mmol) in THE (4 mL) and MeOH (4 mL) using sodium borohydride (55.9 mg, 1.477 mmol) and stirring at RT for 10 min. This gave after workup (7-bromo-4-((1-methyl-1H-pyrazol-3-yl)methoxy)benzofuran-5-yl)methanol (79c) (166 mg, 100% yield) as a white solid; MS (ES+): 359.0, 361.0 (M+Na).

### Step-3: Preparation of 3-(((7-bromo-5-(bromomethyl)benzofuran-4-yl)oxy)methyl)-1-methyl-1H-pyrazole (79d)

Compound 79d was prepared according to the procedure reported in step-3 of scheme 65, from (7-bromo-4-((1-methyl-1H-pyrazol-3-yl)methoxy)benzofuran-5-yl)methanol (79c) (166 mg, 0.492 mmol) in dry DCM (8 mL) using PBr₃ (0.056 mL, 0.591 mmol) and stirring for 90 min. This gave after workup 3-(((7-bromo-5-(bromomethyl)benzofuran-4-yl)oxy)methyl)-1-methyl-1H-pyrazole (79d) (197 mg), which was used as such in the next step; MS (ES+): 420.9, 422.9, 424.9 (M+Na).

### Step-4: Preparation of ethyl 2-(2-((7-bromo-4-((1-methyl-1H-pyrazol-3-yl)methoxy)benzofuran-5-yl)methoxy)phenyl)acetate (79e)

Compound 79e was prepared according to the procedure reported in step-4 of scheme 14, from 3-(((7-bromo-5-(bromomethyl)benzofuran-4-yl)oxy)methyl)-1-methyl-1H-pyrazole (79d) (197 mg, 0.492 mmol) in acetone (10 mL) using ethyl 2-(2-hydroxyphenyl)acetate (1c) (177 mg, 0.985 mmol) and potassium carbonate (272 mg, 1.970 mmol) and stirring overnight at RT. This gave after workup and purification using flash column chromatography [silica gel (12 g) eluting with ethyl acetate/MeOH (9:1) in hexanes from 0-80%]ethyl 2-(2-((7-bromo-4-((1-methyl-1H-pyrazol-3-yl)methoxy)benzofuran-5-yl)methoxy)phenyl)acetate (79e) (172 mg, 70% yield) as a yellow oil; ¹H NMR (300 MHz, DMSO-d₆) δ 8.13 (d, J=2.3 Hz, 1H), 7.64 (d, J=2.2 Hz, 1H), 7.50 (s, 1H), 7.35 (d, J=2.3 Hz, 1H), 7.31-7.11 (m, 2H), 7.03-6.84 (m, 2H), 6.26 (d, J=2.2 Hz, 1H), 5.25 (s, 2H), 5.05 (s, 2H), 3.99 (q, J=7.1 Hz, 2H), 3.79 (s, 3H), 3.60 (s, 2H), 1.06 (t, J=7.1 Hz, 3H); MS (ES+): 521.1, 523.0 (M+Na).

### Step-5: Preparation of ethyl 2-(2-((7-(3-(aminomethyl)phenyl)-4-((1-methyl-1H-pyrazol-3-yl)methoxy)benzofuran-5-yl)methoxy)phenyl)acetate (79f)

Compound 79f was prepared according to the procedure reported in step-7 of scheme 25, from ethyl 2-(2-((7-bromo-4-((1-methyl-1H-pyrazol-3-yl)methoxy)benzofuran-5-yl)methoxy)phenyl)acetate (79e) (86 mg, 0.172 mmol) in dioxane (5 mL) using 3-(aminomethyl)phenylboronic acid hydrochloride (9e) (48.4 mg, 0.258 mmol), bis(triphenylphosphine)palladium(II) chloride (18.13 mg, 0.026 mmol), a solution of K₂CO₃ (71.4 mg, 0.517 mmol) in water (0.5 mL) and stirring at 100° C. for 4.5 h. This gave after workup and purification using flash column chromatography [silica gel (12 g), eluting with DMA-80 in DCM from 0-80%]ethyl 2-(2-((7-(3-(aminomethyl)phenyl)-4-((1-methyl-1H-pyrazol-3-yl)methoxy)benzofuran-5-yl)methoxy)phenyl)acetate (79f) (74 mg, 82% yield) as a dark oil; MS (ES+): 526.2 (M+1).

### Step-6: Preparation of 2-(2-((7-(3-(aminomethyl)phenyl)-4-((1-methyl-1H-pyrazol-3-yl)methoxy)benzofuran-5-yl)methoxy)phenyl)acetic acid (79g)

Compound 79g was prepared according to the procedure reported in step-8 of scheme 1, from ethyl 2-(2-((7-(3-(aminomethyl)phenyl)-4-((1-methyl-1H-pyrazol-3-yl)methoxy)benzofuran-5-yl)methoxy)phenyl)acetate (79f) (74 mg, 0.141 mmol) in MeOH/THF (6 mL, 1:1) using a solution of lithium hydroxide (67 mg, 1.597 mmol) in water (2 mL) and stirring overnight at RT. This gave after workup and purification using reverse phase column chromatography [C18 column (50 g), eluting with ACN in water from 0-100%]2-(2-((7-(3-(aminomethyl)phenyl)-4-((1-methyl-1H-pyrazol-3-yl)methoxy)benzofuran-5-yl)methoxy)phenyl)acetic acid (79g) (50 mg, 71% yield) as a white solid; ¹H NMR (300 MHz, DMSO-d₆) δ 8.17 (s, 1H), 8.13-7.98 (m, 2H), 7.82 (s, 1H), 7.70 (d, J=2.2 Hz, 1H), 7.44 (t, J=7.6 Hz, 1H), 7.38-7.22 (m, 2H), 7.16-6.96 (m, 2H), 6.78 (t, J=7.3 Hz, 1H), 6.69 (d, J=8.1 Hz, 1H), 6.36 (d, J=2.2 Hz, 1H), 5.30 (s, 2H), 5.15 (s, 2H), 3.97 (s, 2H), 3.83 (s, 3H), 3.39 (s, 2H); MS (ES+): 498.2 (M+1); (ES−): 496.2 (M−1); Analysis calculated for: C₂₉H₂₇N₃O₅·0.2HCl: C, 69.00; H, 5.43; N, 8.32; Cl, 1.40; Found: C, 69.02; H, 5.40; N, 8.45; Cl, 1.19.

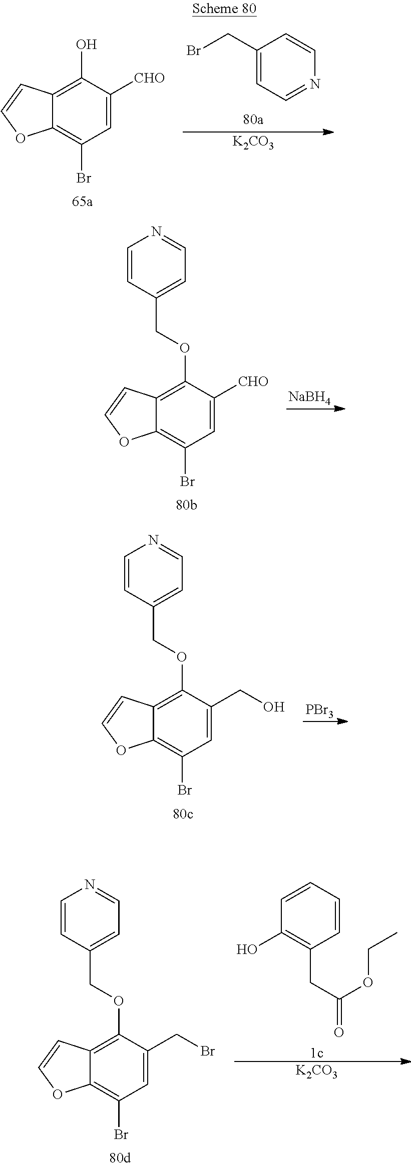

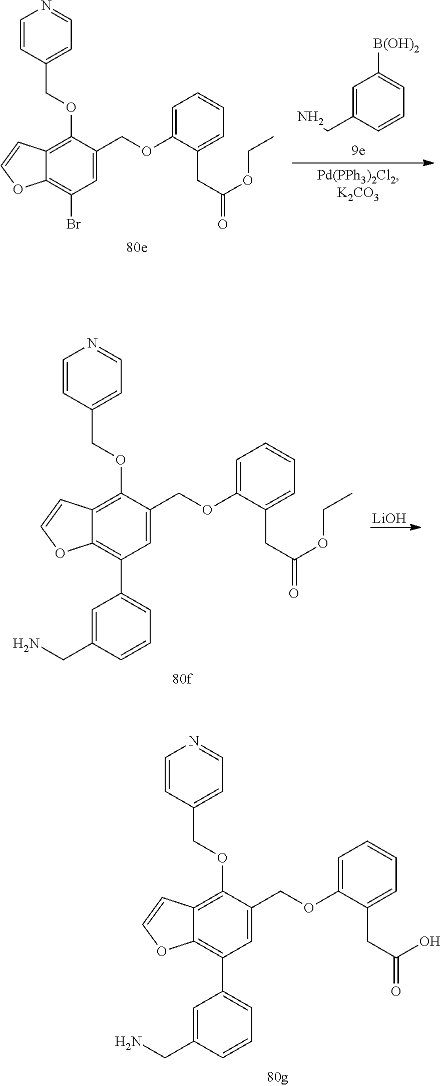

### Preparation of 2-(2-((7-(3-(aminomethyl)phenyl)-4-(pyridin-4-ylmethoxy)benzofuran-5-yl)methoxy)phenyl)acetic acid (80g)

### Step-1: Preparation of 7-bromo-4-(pyridin-4-ylmethoxy)benzofuran-5-carbaldehyde (80b)

Compound 80b was prepared according to the procedure reported in step-4 of scheme 14, from 7-bromo-4-hydroxybenzofuran-5-carbaldehyde (65a) (295 mg, 1.224 mmol) in acetone (8 mL) using 4-(bromomethyl)pyridine, HBr (80a) (619 mg, 2.448 mmol; CAS #54751-01-8), K₂CO₃ (1015 mg, 7.34 mmol) and stirring at RT for 16 h. This gave after workup and purification using flash column chromatography [silica gel (12 g), eluting with EtOAc/MeOH (9:1) in hexane from 0-90%]7-bromo-4-(pyridin-4-ylmethoxy)benzofuran-5-carbaldehyde (80b) (172 mg, 42% yield) as a white yellow solid; ¹H NMR (300 MHz, DMSO-d₆) δ 10.38 (s, 1H), 8.66-8.56 (m, 2H), 8.28 (d, J=2.4 Hz, 1H), 7.85 (s, 1H), 7.63 (d, J=2.4 Hz, 1H), 7.61-7.53 (m, 2H), 5.67 (s, 2H); MS (ES+): 331.9, 334.0, (M+H).

### Step-2: Preparation of (7-bromo-4-(pyridin-4-ylmethoxy)benzofuran-5-yl)methanol (80c)

Compound 80c was prepared according to the procedure reported in step-3 of scheme 5, from 7-bromo-4-(pyridin-4-ylmethoxy)benzofuran-5-carbaldehyde (80b) (170 mg, 0.512 mmol) in THE (3 mL) and MeOH (3 mL) using sodium borohydride (58.1 mg, 1.535 mmol) and stirring at RT for 60 min. This gave after workup (7-bromo-4-(pyridin-4-ylmethoxy)benzofuran-5-yl)methanol (80c) (171 mg) as a white solid; MS (ES+) 334.0, 336.0 (M+1).

### Step-3: Preparation of 4-(((7-bromo-5-(bromomethyl)benzofuran-4-yl)oxy)methyl)pyridine (80d)

Compound 80d was prepared according to the procedure reported in step-3 of scheme 65, from (7-bromo-4-(pyridin-4-ylmethoxy)benzofuran-5-yl)methanol (80c) (171 mg, 0.512 mmol) in dry DCM (8 mL) using PBr₃ (0.058 mL, 0.614 mmol) and stirring for 90 min. This gave after workup 4-(((7-bromo-5-(bromomethyl)benzofuran-4-yl)oxy)methyl)pyridine (80d) (203 mg) and was used as such in the next step; MS (ES+): 395.9, 397.9, 399.9 (M+1).

### Step-4: Preparation of ethyl 2-(2-((7-bromo-4-(pyridin-4-ylmethoxy)benzofuran-5-yl)methoxy)phenyl)acetate (80e)

Compound 80e was prepared according to the procedure reported in step-4 of scheme 14, from 4-(((7-bromo-5-(bromomethyl)benzofuran-4-yl)oxy)methyl)pyridine (80d) (203 mg, 0.511 mmol) in acetone (10 mL) using ethyl 2-(2-hydroxyphenyl)acetate (1c) (184 mg, 1.023 mmol), potassium carbonate (283 mg, 2.045 mmol) and stirring at RT for 72 h. This gave after workup and purification using flash column chromatography [silica gel (12 g) eluting with ethyl acetate in hexanes from 0-100%]ethyl 2-(2-((7-bromo-4-(pyridin-4-ylmethoxy)benzofuran-5-yl)methoxy)phenyl)acetate (80e) (54 mg, 21% yield) as a yellow solid; ¹H NMR (300 MHz, DMSO-d₆) δ 8.58-8.50 (m, 2H), 8.15 (d, J=2.2 Hz, 1H), 7.61-7.57 (m, 1H), 7.53-7.45 (m, 2H), 7.45-7.38 (m, 1H), 7.29-7.18 (m, 2H), 7.06 (d, J=8.3 Hz, 1H), 6.93 (t, J=7.3 Hz, 1H), 5.47 (s, 2H), 5.18 (s, 2H), 3.95 (q, J=7.1 Hz, 2H), 3.57 (s, 2H), 1.02 (t, J=7.1 Hz, 3H); MS (ES+): 496.1, 498.0 (M+1).

### Step-5: Preparation of ethyl 2-(2-((7-(3-(aminomethyl)phenyl)-4-(pyridin-4-ylmethoxy)benzofuran-5-yl)methoxy)phenyl)acetate (80f)

Compound 80f was prepared according to the procedure reported in step-7 of scheme 25, from ethyl 2-(2-((7-bromo-4-(pyridin-4-ylmethoxy)benzofuran-5-yl)methoxy)phenyl)acetate (80e) (52 mg, 0.105 mmol) in dioxane (5 mL) using 3-(aminomethyl)phenylboronic acid hydrochloride (9e) (29.4 mg, 0.157 mmol), bis(triphenylphosphine)palladium(II) chloride (11.03 mg, 0.016 mmol), a solution of K₂CO₃ (43.4 mg, 0.314 mmol) in water (0.5 mL) and stirring at 100° C. for 4.5 h. This gave after workup and purification using flash column chromatography [silica gel (12 g), eluting with DMA-80 in DCM from 0-80%]ethyl 2-(2-((7-(3-(aminomethyl)phenyl)-4-(pyridin-4-ylmethoxy)benzofuran-5-yl)methoxy)phenyl)acetate (80f) (54 mg, 99% yield) as a dark oil; MS (ES+): 523.2 (M+1).

### Step-6: Preparation of 2-(2-((7-(3-(aminomethyl)phenyl)-4-(pyridin-4-ylmethoxy)benzofuran-5-yl)methoxy)phenyl)acetic acid (80g)

Compound 80g was prepared according to the procedure reported in step-8 of scheme 1, from ethyl 2-(2-((7-(3-(aminomethyl)phenyl)-4-(pyridin-4-ylmethoxy)benzofuran-5-yl)methoxy)phenyl)acetate (80f) (53 mg, 0.101 mmol) in MeOH/THF (4 mL, 1:1) using a solution of lithium hydroxide (42 mg, 1.001 mmol) in water (1.5 mL) and stirring overnight at RT. This gave after workup and purification using reverse phase column chromatography [C18 column (50 g), eluting with ACN in water from 0-100%]2-(2-((7-(3-(aminomethyl)phenyl)-4-(pyridin-4-ylmethoxy)benzofuran-5-yl)methoxy)phenyl)acetic acid (80g) (22 mg, 44% yield) as a white solid; ¹H NMR (300 MHz, DMSO-d₆) δ 8.71-8.58 (m, 2H), 8.19 (s, 1H), 8.09 (d, J=2.3 Hz, 1H), 8.03 (d, J=7.9 Hz, 1H), 7.89 (s, 1H), 7.59 (d, J=5.4 Hz, 2H), 7.45 (t, J=7.7 Hz, 1H), 7.36-7.26 (m, 2H), 7.14-7.01 (m, 2H), 6.80 (t, J=7.9 Hz, 2H), 5.51 (s, 2H), 5.30 (s, 2H), 3.98 (s, 2H), 3.40 (s, 2H); MS (ES+): 495.2 (M+1); (ES−): 493.1 (M−1).

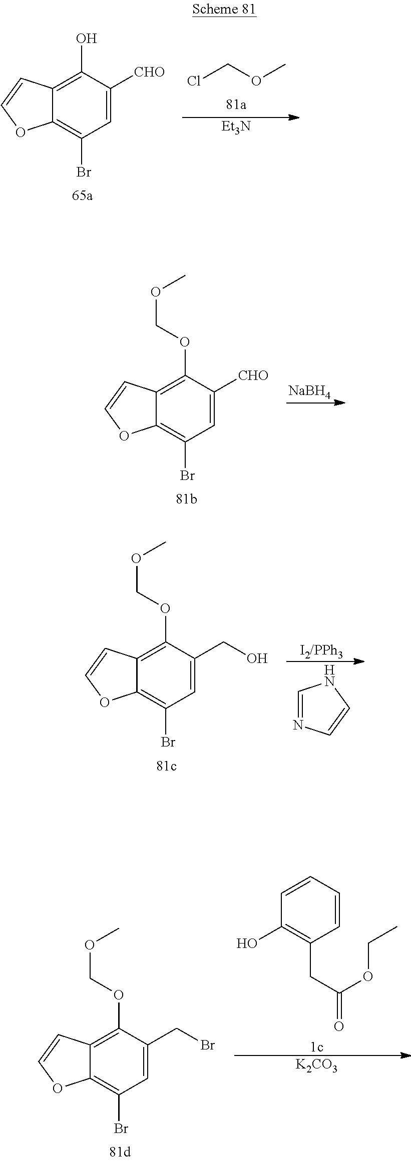

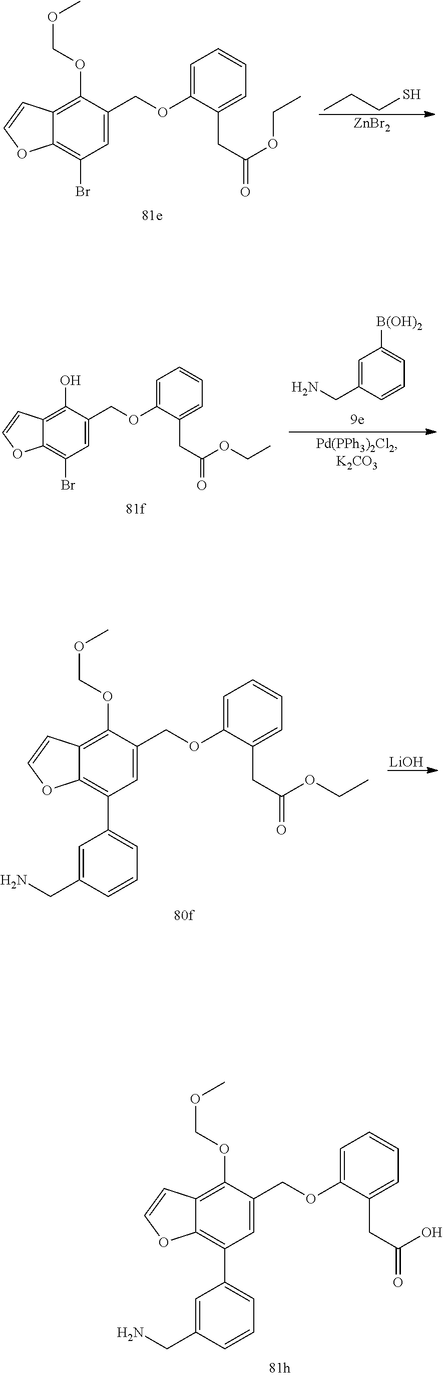

### Preparation of 2-(2-((7-(3-(aminomethyl)phenyl)-4-(methoxymethoxy)benzofuran-5-yl)methoxy)phenyl)acetic acid (81h)

### Step-1: Preparation of 7-bromo-4-(methoxymethoxy)benzofuran-5-carbaldehyde (81b)

To a stirred solution of 7-bromo-4-hydroxybenzofuran-5-carbaldehyde (65a) (1.62 g, 6.72 mmol) in DCM (30 mL) was added chloro(methoxy)methane (81a) (1.082 g, 13.44 mmol) at 0° C. followed by slow addition of triethylamine (TEA) (2.81 mL, 20.16 mmol) and the reaction mixture was allowed to warm to room temperature over a period of 3 h. The mixture was diluted with DCM (30 mL) quenched with saturated aqueous NH₄Cl and stirred at RT for 10 min. The organic phase was separated and washed with brine, dried, filtered and concentrated in vacuum to afford 7-bromo-4-(methoxymethoxy)benzofuran-5-carbaldehyde (81b) (1.91 g) as a yellow solid, which was used as such for next step; ¹H NMR (300 MHz, DMSO-d₆) δ 10.37 (s, 1H), 8.25 (d, J=2.3 Hz, 1H), 7.84 (s, 1H), 7.40 (d, J=2.3 Hz, 1H), 5.50 (s, 2H), 3.53 (s, 3H).

### Step-2: Preparation of (7-bromo-4-(methoxymethoxy)benzofuran-5-yl)methanol (81c)

Compound 81c was prepared according to the procedure reported in step-3 of scheme 5, from 7-bromo-4-(methoxymethoxy)benzofuran-5-carbaldehyde (81b) (1.89 g, 6.63 mmol) in THE (8 mL) and MeOH (8 mL) using sodium borohydride (502 mg, 13.26 mmol) and stirring at RT for 15 min. This gave after workup (7-bromo-4-(methoxymethoxy)benzofuran-5-yl)methanol (81c) (1.75 g, 92% yield) as a colorless oil, which solidified to a white solid on standing; ¹H NMR (300 MHz, DMSO-d₆) δ 8.06 (d, J=2.3 Hz, 1H), 7.55 (s, 1H), 7.17 (d, J=2.3 Hz, 1H), 5.28 (s, 2H), 5.23 (t, J=5.7 Hz, 1H), 4.69-4.54 (m, 2H), 3.49 (s, 3H).

### Step-3: Preparation of 7-bromo-5-(iodomethyl)-4-(methoxymethoxy)benzofuran (81d)

To a stirred solution of triphenylphosphine (1.317 g, 5.02 mmol) in DCM (30 mL) was added imidazole (0.342 g, 5.02 mmol), iodine (1.275 g, 5.02 mmol), followed by a solution of (7-bromo-4-(methoxymethoxy)benzofuran-5-yl)methanol (81c) (1.03 g, 3.59 mmol) in DCM (10 ml) at RT and stirring at RT for 45 min. The reaction was diluted with DCM (40 mL) and washed with 20% aqueous solution of NaS₂O₃ (40 mL) and the aqueous layer was extracted with DCM (30 mL). The combined organics were washed, dried, filtered and concentrated partially in vacuum to furnish a solution of 7-bromo-5-(iodomethyl)-4-(methoxymethoxy)benzofuran (81d) (˜20 mL) in DCM, which was used as such for the next step.

### Step-4: Preparation of ethyl 2-(2-((7-bromo-4-(methoxymethoxy)benzofuran-5-yl)methoxy)phenyl)acetate (81e)

Compound 81e was prepared according to the procedure reported in step-4 of scheme 14, from 7-bromo-5-(iodomethyl)-4-(methoxymethoxy)benzofuran (81d) (˜20 mL solution in DCM from step-3 above) in acetone (30 mL) using ethyl 2-(2-hydroxyphenyl)acetate (1c) (752 mg, 4.17 mmol), K₂CO₃ (1.441 g, 10.43 mmol) and stirring overnight at RT. This gave after workup and purification using flash column chromatography [silica gel (24 g), eluting with EtOAc in hexane from 0-60%]ethyl 2-(2-((7-bromo-4-(methoxymethoxy)benzofuran-5-yl)methoxy)phenyl)acetate (81e) (1.25 g, 80% yield) as a pale yellow oil; ¹H NMR (300 MHz, DMSO-d₆) δ 8.12 (d, J=2.3 Hz, 1H), 7.58 (s, 1H), 7.32-7.19 (m, 3H), 7.11 (d, J=3.8 Hz, 1H), 6.93 (td, J=7.4, 1.1 Hz, 1H), 5.33 (s, 2H), 5.17 (s, 2H), 3.98 (q, J=7.1 Hz, 2H), 3.61 (s, 2H), 3.49 (s, 3H), 1.06 (t, J=7.1 Hz, 3H).

### Step-5: Preparation of ethyl 2-(2-((7-bromo-4-hydroxybenzofuran-5-yl)methoxy)phenyl)acetate (81f)

To a stirred solution of ethyl 2-(2-((7-bromo-4-(methoxymethoxy)benzofuran-5-yl)methoxy)phenyl)acetate (81e) (900 mg, 2.003 mmol) in DCM (45 mL) was added zinc(II) bromide (677 mg, 3.00 mmol) and propane-1-thiol (229 mg, 3.00 mmol) at 0° C. and the mixture was stirred at 0° C. for 28 min. The resulting mixture was diluted with DCM (50 mL) and saturated NaHCO₃ (40 mL) at 0° C. The organic layer was separated, and the aqueous layer was extracted with DCM (60 mL). The combined organics were dried, filtered and concentrated in vacuo. The residue obtained was purified using flash column chromatography [silica gel (24 g), eluting with EtOAc in hexanes from 0-40%] to provide ethyl 2-(2-((7-bromo-4-hydroxybenzofuran-5-yl)methoxy)phenyl)acetate (81f) (262 mg, 32% yield) as a yellow solid; ¹H NMR (300 MHz, DMSO-d₆) δ 10.36 (s, 1H), 7.98 (d, J=2.2 Hz, 1H), 7.43 (s, 1H), 7.34-7.17 (m, 3H), 7.10 (d, J=8.1 Hz, 1H), 6.98-6.87 (m, 1H), 5.11 (s, 2H), 4.00 (q, J=7.1 Hz, 2H), 3.60 (s, 2H), 1.07 (t, J=7.1 Hz, 3H).

### Step-6: Preparation of ethyl 2-(2-((7-(3-(aminomethyl)phenyl)-4-(methoxymethoxy)benzofuran-5-yl)methoxy)phenyl)acetate (81g)

Compound 81g was prepared according to the procedure reported in step-7 of scheme 25, from a mixture of ethyl 2-(2-((7-bromo-4-(methoxymethoxy)benzofuran-5-yl)methoxy)phenyl)acetate (81e) (246 mg, 0.548 mmol) and ethyl 2-(2-((7-bromo-4-hydroxybenzofuran-5-yl)methoxy)phenyl)acetate (81f) (186 mg, 0.459 mmol) in dioxane (15 mL) using 3-(aminomethyl)phenylboronic acid hydrochloride (9e) (281 mg, 1.5 mmol), bis(triphenylphosphine)palladium(II) chloride (105 mg, 0.15 mmol), a solution of K₂CO₃ (415 mg, 3 mmol) in water (1.5 mL) and stirring at 100° C. for 3.5 h. This gave after workup and purification using flash column chromatography [silica gel (12 g), eluting with DMA-80 in DCM from 0-90%]ethyl 2-(2-((7-(3-(aminomethyl)phenyl)-4-(methoxymethoxy)benzofuran-5-yl)methoxy)phenyl)acetate (81g) (176 mg, 68% yield) as a brown oil; MS (ES+): 476.20 (M+1).

### Step-7: Preparation of 2-(2-((7-(3-(aminomethyl)phenyl)-4-(methoxymethoxy)benzofuran-5-yl)methoxy)phenyl)acetic acid (81h)

Compound 81h was prepared according to the procedure reported in step-8 of scheme 1, from ethyl 2-(2-((7-(3-(aminomethyl)phenyl)-4-(methoxymethoxy)benzofuran-5-yl)methoxy)phenyl)acetate (81g) (88 mg, 0.185 mmol) in MeOH/THF (4 mL, 1:1) using a solution of lithium hydroxide (31.1 mg, 0.740 mmol) in water (2 mL) and stirring overnight at RT. This gave after workup and purification using reverse phase column chromatography [C18 column (50 g), eluting with ACN in water from 0-100%]2-(2-((7-(3-(aminomethyl)phenyl)-4-(methoxymethoxy)benzofuran-5-yl)methoxy)phenyl)acetic acid (81h) (36 mg, 44% yield) as a white solid; ¹H NMR (300 MHz, DMSO-d₆) δ 8.29-8.19 (m, 1H), 8.11-8.01 (m, 2H), 7.90 (s, 1H), 7.44 (t, J=7.7 Hz, 1H), 7.30 (d, J=7.6 Hz, 1H), 7.17 (d, J=2.3 Hz, 1H), 7.13-7.01 (m, 2H), 6.88 (d, J=8.1 Hz, 1H), 6.80 (t, J=7.4 Hz, 1H), 5.39 (s, 2H), 5.32 (s, 2H), 3.98 (s, 2H), 3.58 (s, 3H), 3.39 (s, 2H); MS (ES+): 448.2 (M+1); MS (ES−): 446.2 (M−1); Analysis calculated for: C₂₆H₂₅NO₆·0.35HCl·1.25H₂O: C, 64.69; H, 5.81; N, 2.90; Cl, 2.57; Found: C, 64.41; H, 5.76; N, 2.97; Cl, 2.44.

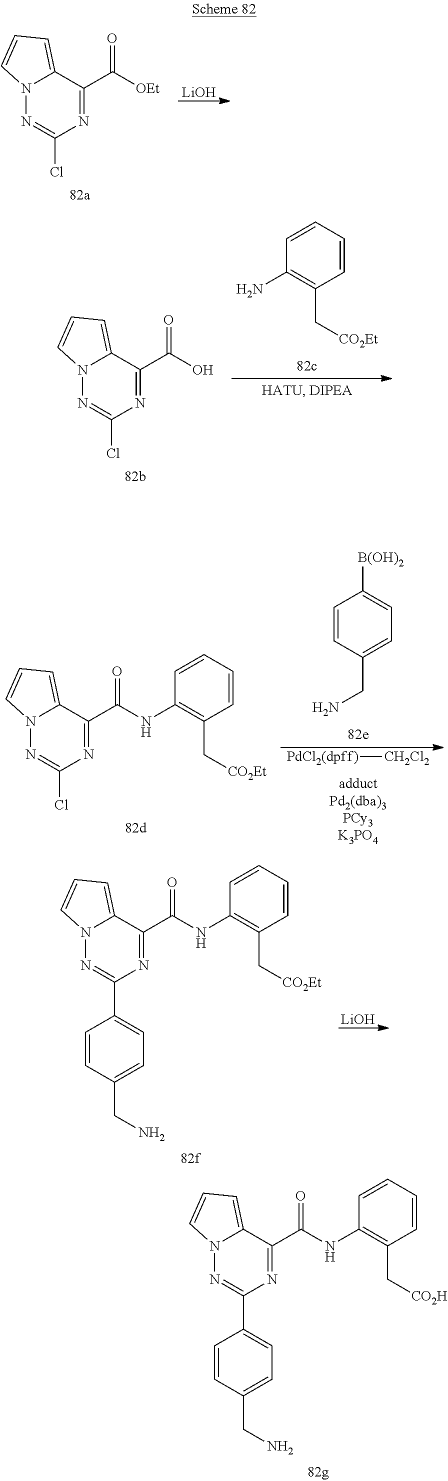

### Preparation of 2-(2-(2-(4-(aminomethyl)phenyl)pyrrolo[2,1-f][1,2,4]triazine-4-carboxamido)phenyl)acetic acid (82g)

### Step-1: Preparation of 2-chloropyrrolo[2,1-f][1,2,4]triazine-4-carboxylic acid (82b)

Compound 82b was prepared according to procedure reported in step-8 of scheme 1, from ethyl 2-chloropyrrolo[2,1-f][1,2,4]triazine-4-carboxylate (82a) (0.700 g, 3.10 mmol; CAS #2255366-50-6) in THE (5 mL) using a solution of lithium hydroxide, 2M (2.327 mL, 4.65 mmol) and stirring for 16 h at RT. This gave after workup 2-chloropyrrolo[2,1-f][1,2,4]triazine-4-carboxylic acid (82b) (0.48 g, 78% yield) as a red solid; ¹H NMR (300 MHz, DMSO-d₆) δ 8.47-8.26 (m, 1H), 7.44-7.30 (m, 1H), 7.22 (dd, J=4.8, 2.5 Hz, 1H); MS (ES+): 198/200 (M+1); (ES−): 196/198 (M−1).

### Step-2: Preparation of ethyl 2-(2-(2-chloropyrrolo[2,1-f][1,2,4]triazine-4-carboxamido)phenyl)acetate (82d)

To a solution of 2-chloropyrrolo[2,1-f][1,2,4]triazine-4-carboxylic acid (82b) (0.48 g, 2.429 mmol), ethyl 2-(2-aminophenyl)acetate (82c) (0.435 g, 2.429 mmol; CAS #64460-85-1) and HATU (1.386 g, 3.64 mmol) in DMF (15 mL) was added DIPEA (1.570 g, 12.15 mmol) dropwise and the mixture was stirred at RT for 16 h. The resulting solution was diluted with H₂O (30 mL), extracted with EtOAc (30 mL×3). The combined organics were washed with 0.5 M NaOH (25 mL), H₂O (25 mL×3), brine (25 mL), dried, filtered and concentrated in vacuo. The obtained residue was purified using flash column chromatography [SiO₂ gel (24 g), eluting with EtOAc in hexane from 0-8%]ethyl 2-(2-(2-chloropyrrolo[2,1-f][1,2,4]triazine-4-carboxamido)phenyl)acetate (82d) (0.66 g, 76% yield) as a yellow solid; H NMR (300 MHz, DMSO-d₆) δ 10.51 (s, 1H), 8.44 (dd, J=2.5, 1.3 Hz, 1H), 7.73-7.62 (m, 1H), 7.59 (dd, J=4.7, 1.3 Hz, 1H), 7.46-7.34 (m, 2H), 7.34-7.23 (m, 2H), 4.07 (q, J=7.1 Hz, 2H), 3.80 (s, 2H), 1.12 (t, J=7.1 Hz, 3H); MS (ES+): 359/361 (M+1); (ES−): 357/359 (M−1).

### Step-3: Preparation of ethyl 2-(2-(2-(4-(aminomethyl)phenyl)pyrrolo[2,1-f][1,2,4]triazine-4-carboxamido)phenyl)acetate (82f)

Compound 82f was prepared according to the procedure reported in step-7 of scheme 25, from ethyl 2-(2-(2-chloropyrrolo[2,1-f][1,2,4]triazine-4-carboxamido)phenyl)acetate (82d) (100 mg, 0.279 mmol) in dioxane (4 mL) using (4-(aminomethyl)phenyl)boronic acid hydrochloride (82e) (62.7 mg, 0.334 mmol), PdCl₂(dppf)-CH₂Cl₂ adduct (22.73 mg, 0.028 mmol), Pd₂(dba)₃ (25.5 mg, 0.028 mmol), PCy₃ (15.63 mg, 0.056 mmol), 2 M aqueous solution of K₃PO₄ (0.418 mL, 0.836 mmol) and stirring at 100° C. for 4 h. This gave after workup and purification using flash column chromatography [silica gel (12 g), eluting with MeOH in DCM from 0-5%]ethyl 2-(2-(2-(4-(aminomethyl)phenyl)pyrrolo[2,1-f][1,2,4]triazine-4-carboxamido)phenyl)acetate (82f) (95 mg, 79% yield) as a pale-yellow oil; MS (ES+): 430.2 (M+1); (ES−): 428.10 (M−1).

### Step-4: Preparation of 2-(2-(2-(4-(aminomethyl)phenyl)pyrrolo[2,1-f][1,2,4]triazine-4-carboxamido)phenyl)acetic acid (82g)

Compound 82g was prepared according to the procedure reported in step-8 of scheme 1, from ethyl 2-(2-(2-(4-(aminomethyl)phenyl)pyrrolo[2,1-f][1,2,4]triazine-4-carboxamido)phenyl)acetate (82f) (95 mg, 0.221 mmol) in EtOH (5 mL) using a solution of lithium hydroxide, 2M (0.553 mL, 1.106 mmol) and stirring at RT for 16 h. This gave after workup and purification using reverse phase column chromatography [C18 column (100 g), eluting with ACN in water (containing 0.1% HCl) from 0-100%]2-(2-(2-(4-(aminomethyl)phenyl)pyrrolo[2,1-f][1,2,4]triazine-4-carboxamido)phenyl)acetic acid (82g) (64 mg, 72% yield) as a yellow solid; ¹H NMR (300 MHz, DMSO-d₆) δ 10.85 (s, 1H, D₂O exchangeable), 8.49 (d, J=8.2 Hz, 2H), 8.40 (bs, 2H, D₂O exchangeable), 8.34-8.28 (m, 1H), 7.78 (d, J=7.7 Hz, 1H), 7.61 (d, J=8.2 Hz, 2H), 7.53 (dd, J=4.6 Hz, 1H), 7.39-7.28 (m, 2H), 7.25-7.14 (m, 2H), 4.07 (s, 2H), 3.72 (s, 2H); MS (ES+): 402 (M+1); (ES−): 400 (M−1).

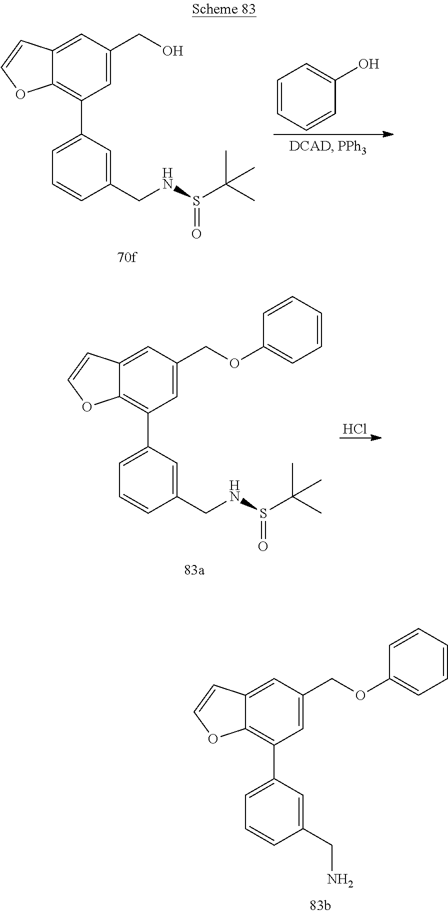 [SMILES: CC(C)(C)[S@@](=O)NCc1cccc(-c2cc(CO)cc3ccoc23)c1 ; Oc1ccccc1 ; CC(C)(C)[S@@](=O)NCc1cccc(-c2cc(COc3ccccc3)cc3ccoc23)c1 ; Cl ; NCc1cccc(-c2cc(COc3ccccc3)cc3ccoc23)c1]

### Preparation of (3-(5-(phenoxymethyl)benzofuran-7-yl)phenyl)methanamine (83b)

### Step-1: Preparation of 2-methyl-N-(3-(5-(phenoxymethyl)benzofuran-7-yl)benzyl)propane-2-sulfinamide (83a)

Compound 83a was prepared according to procedure reported in step-2 of scheme 1, from N-(3-(5-(hydroxymethyl)benzofuran-7-yl)benzyl)-2-methylpropane-2-sulfinamide (70f) (300 mg, 0.839 mmol) in DCM (15 mL) using triphenylphosphine (330 mg, 1.259 mmol), phenol (79 mg, 0.839 mmol), and a solution of (E)-bis(4-chlorobenzyl) diazene-1,2-dicarboxylate (DCAD, 462 mg, 1.259 mmol) in DCM (15 mL) and stirring at room temperature for 2 h. This gave after workup and purification by flash column chromatography [silica gel (80 g), eluting with EtOAc/MeOH (9:1) in hexanes from 0-50%] followed by [silica gel (24 g), eluting with EtOAc in hexane from 0-50%]2-methyl-N-(3-(5-(phenoxymethyl)benzofuran-7-yl)benzyl)propane-2-sulfinamide (83a) (152 mg, 42% yield); MS (ES+): 434.20 (M+1).

### Step-2: Preparation of (3-(5-(phenoxymethyl)benzofuran-7-yl)phenyl)methanamine (83b)

Compound 83b was prepared according to procedure reported in step-7 of scheme 1, from 2-methyl-N-(3-(5-(phenoxymethyl)benzofuran-7-yl)benzyl)propane-2-sulfinamide (83a) (100 mg, 0.231 mmol) in THE (15 mL) using 3M aqueous HCl (0.231 mL, 0.693 mmol) and stirring at RT for 14 h. This gave after workup and purification using reverse phase column chromatography [C18 column (50 g), eluting with ACN in water (containing 0.1% HCl) from 0-100%] to afford (3-(5-(phenoxymethyl)benzofuran-7-yl)phenyl)methanamine (83b) (12 mg, 16% yield) HCl salt as a white solid; ¹H NMR (300 MHz, DMSO-d₆) δ 8.39 (s, 3H, D₂O exchangeable), 8.11 (d, J=2.2 Hz, 1H), 8.00-7.96 (m, 1H), 7.92 (dt, J=7.4, 1.7 Hz, 1H), 7.78 (d, J=1.6 Hz, 1H), 7.65 (d, J=1.7 Hz, 1H), 7.63-7.51 (m, 2H), 7.35-7.25 (m, 2H), 7.11-7.02 (m, 3H), 6.94 (tt, J=7.3, 1.1 Hz, 1H), 5.25 (s, 2H), 4.13 (s, 2H); MS (ES+): 330.15 (M+1).

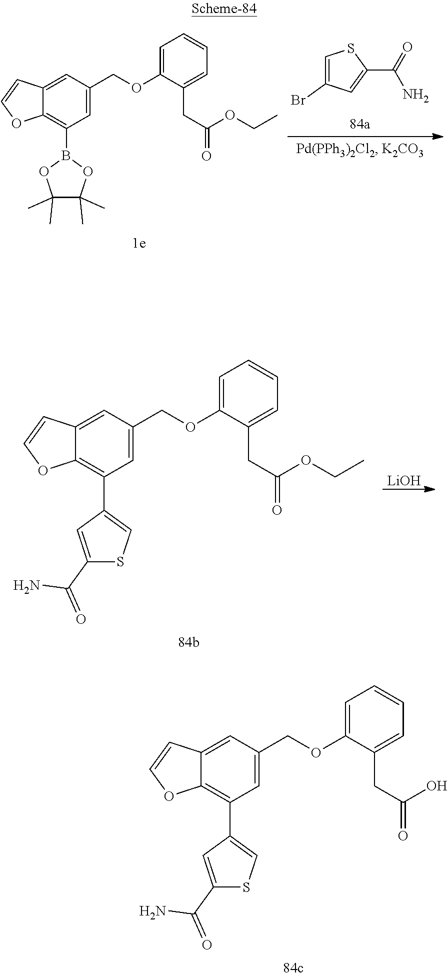

### Preparation of 2-(2-((7-(5-carbamoylthiophen-3-yl)benzofuran-5-yl)methoxy)phenyl)acetic acid (84c)

### Step-1: Preparation of ethyl 2-(2-((7-(5-carbamoylthiophen-3-yl)benzofuran-5-yl)methoxy)phenyl)acetate (84b)

Compound 84b was prepared according to the procedure reported in step-4 of scheme-1, from ethyl 2-(2-((7-(4,4,5,5-tetramethyl-1,3,2-dioxaborolan-2-yl)benzofuran-5-yl)methoxy)phenyl)acetate (1e) (500 mg, 1.146 mmol) in dioxane (6 mL) using 4-bromothiophene-2-carboxamide (84a) (236 mg, 1.146 mmol; CAS #83933-17-9), bis(triphenylphosphine)palladium(II) chloride (121 mg, 0.172 mmol), a solution of K₂CO₃ (475 mg, 3.44 mmol) in water (2 mL) and heating at 90° C. for 3 h. This gave after workup and purification using flash column chromatography [silica gel (12 g), eluting with DMA-80 in DCM from 0-50%]ethyl 2-(2-((7-(5-carbamoylthiophen-3-yl)benzofuran-5-yl)methoxy)phenyl)acetate (84b) (499 mg, 100% yield) as a yellow syrup; MS (ES+): 458.05 (M+Na).

### Step-2: Preparation of 2-(2-((7-(5-carbamoylthiophen-3-yl)benzofuran-5-yl)methoxy)phenyl)acetic acid (84c)

Compound 84c was prepared according to procedure reported in step-8 of scheme-1, from ethyl 2-(2-((7-(5-carbamoylthiophen-3-yl)benzofuran-5-yl)methoxy)phenyl)acetate (84b) (0.5 g, 1.148 mmol) in THF (3 mL) and methanol (3 mL) using lithium hydroxide hydrate (0.385 g, 9.19 mmol) in water (2 mL) and stirring for 16 h at room temperature. This gave after workup and purification using reverse-phase column chromatography [C-18 column (50 g), eluting with ACN in water (containing 0.1% HCl) from 0-100%]2-(2-((7-(5-carbamoylthiophen-3-yl)benzofuran-5-yl)methoxy)phenyl)acetic acid (84c) (0.02 g, 4% yield) HCl salt as a white solid; ¹H NMR (300 MHz, DMSO-d₆) δ 12.22 (s, 1H), 8.44 (d, J=1.5 Hz, 1H), 8.31 (d, J=1.4 Hz, 1H), 8.13 (d, J=2.2 Hz, 1H), 8.06 (s, 1H), 7.75-7.68 (m, 2H), 7.67-7.60 (m, 1H), 7.27-7.18 (m, 2H), 7.12-7.06 (m, 1H), 7.05 (d, J=2.2 Hz, 1H), 6.90 (td, J=7.4, 1.1 Hz, 1H), 5.25 (s, 2H), 3.60 (s, 2H); MS (ES−): 406.05 (M−1).

### Example 85

The IC₅₀ value of a compound (i.e., the concentration of the compound that inhibits 50% of the enzymatic activity) was calculated according to the procedure reported in U.S. Pat. No. 6,653,340 B1, e.g., column 74 (incorporated by reference).

Specifically, the compounds were dissolved in a stock solution of DMSO at 10.0 or 100 mM. A portion of this stock solution was added to assay buffer in a final volume of 50 μL. Controls included buffer alone and enzyme solutions to which DMSO was added. Substrate was added to the reaction wells immediately or after incubation at room temperature. The reaction rates were measured spectrophotometrically by the generation of product at 405 nm for 600 sec. Background absorbance at 690 nm was measured and subtracted from the absorbance at 405 nm for each well.

The reaction rate for enzyme alone was compared to the rate of enzyme in the presence of inhibitor and the percent inhibition was calculated as shown below:

$$Percent Inhibition = [ Rate without inhibitor - Rate with inhibitor ) / ( Rate without inhibitor ) ] \times 100$$

### Factor D Esterolytic Assay:

An established esterolytic assay for the measurement of Factor D activity and inhibition of Factor D activity was used (Kam, C. M.; McRae, B. J.; Harper, J. W.; Niemann, M. A.; Volanakis, J. E.; Powers, J. C. Human complement proteins D, C2, and B Active site mapping with peptide thioester substrates. J Biol. Chem. 1987, 262, 3444-3451). For this assay Z-Lys-SBzl, 1.29 mM (Kim, S.; Narayana, S. V. L; Volanakis, J. E. Mutational analysis of the substrate binding site of human complement Factor D. Biochemistry. 1994, 33, 14393-14399.) was used as the substrate for Factor D (104 mM). Hydrolysis of this compound by Factor D liberated a free sulfhydryl group which is then reacted with 5,5′-dithiobis(2nitrobenzoic acid) producing an intense yellow color (Habeeb, A. F. S. A. Reaction of protein sulfhydryl groups with Ellman's Reagent. Methods in Enzymol. 1976, 25, 457-464). The assays were performed in 96 well microtiter plates and rates of hydrolysis were monitored at 405 nm on a Biotek Synergy H1 plate reader. Hydrolysis rates were reported as change in mOD/min. The assay was conducted in 100 mM HEPES, 500 mM NaCl, pH 7.5 containing 10% DMSO in a final volume of 50 μL per well.

An IC₅₀, a compound concentration which inhibits 50% of the enzymatic activity, was calculated. Compounds in the examples were tested a minimum of three times. In the table below, three plus symbols (+++) are used to indicate compounds with an IC₅₀ value of less than 1 micromolar; two plus symbols (++) indicate compounds with an IC₅₀ value between 1 and 10 micromolar; and one plus symbol (+) indicates compounds with an IC₅₀ value greater than 10 micromolar.

TABLE 1
Measured Ki (IC₅₀) Value for Compounds.

| Compound | IC₅₀ | Compound | IC₅₀ | Compound | IC₅₀ |
|---|---|---|---|---|---|
| 42e | +++ | 16f | +++ | 51f | +++ |
| 43d | +++ | 28c | +++ | 4c | + |
| 59i | +++ | 29c | +++ | 18c | +++ |
| 11m | +++ | 30c | +++ | 7e | ++ |
| 22h | +++ | 24b | +++ | 9d | +++ |
| 38c | +++ | 31d | +++ | 52f | +++ |
| 12j | +++ | 1j | ++ | 49d | +++ |
| 26f | +++ | 32c | +++ | 53d | +++ |
| 27c | +++ | 2f | +++ | 8g | ++ |
| 44d | +++ | 41e | ++ | 35e | +++ |
| 14k | +++ | 21c | +++ | 19i | +++ |
| 13f | +++ | 25h | +++ | 58b | +++ |
| 45b | +++ | 33c | +++ | 56f | +++ |
| 46d | +++ | 3c | ++ | 57b | +++ |
| 47c | +++ | 5e | +++ | 10d | +++ |
| 50e | + | 36f | +++ | 40g | +++ |
| 23h | +++ | 39e | ++ | 54g | +++ |
| 48g | +++ | 37d | +++ | 55e | +++ |
| 15f | +++ | 6e | +++ | 20h | +++ |
| 61a | +++ | 34c | +++ | 62g | +++ |
| 60a | +++ | 17d | +++ | 63f | ++ |
| 66f | +++ | 73c | +++ | 69g | +++ |
| 64h | +++ | 74d | +++ | 79g | +++ |
| 70f | +++ | 65g | +++ | 80g | +++ |
| 83b | + | 75d | +++ | 81h | +++ |
| 72e | +++ | 76e | +++ | 82g | + |
| 67f | +++ | 78e | +++ | 71e | +++ |
| 68g | +++ | 77e | +++ | 77g | +++ |
| | | 84c | + | 77f | +++ |

Three (+++) is used to denote compounds with an IC₅₀ value of less than 1 micromolar concentration; Two (++) indicates compounds with an IC₅₀ value between 1 and 10 micromolar concentration; One (+) indicate compounds with an IC₅₀ value greater than 10 micromolar concentration.

### INCORPORATION BY REFERENCE

All of the U.S. patents and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A compound of formula (I), or a pharmaceutically acceptable salt thereof: 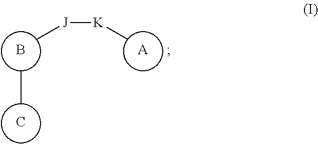
wherein:
\textcircled{A} is selected from the group consisting of 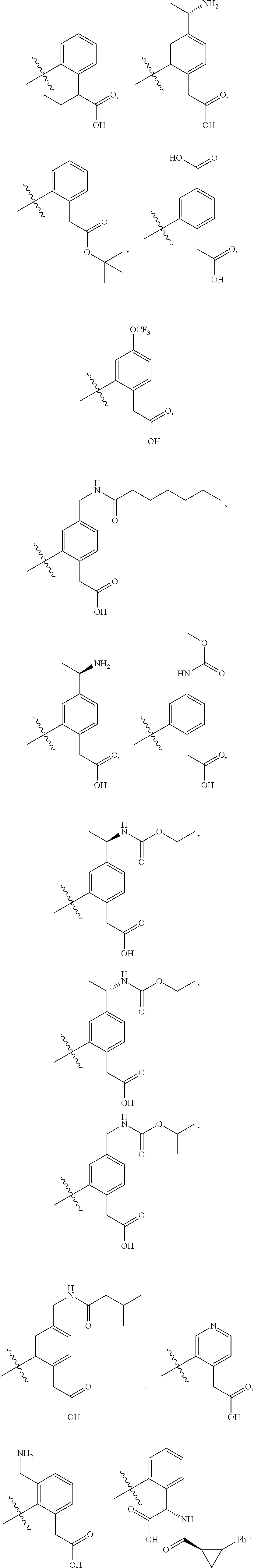 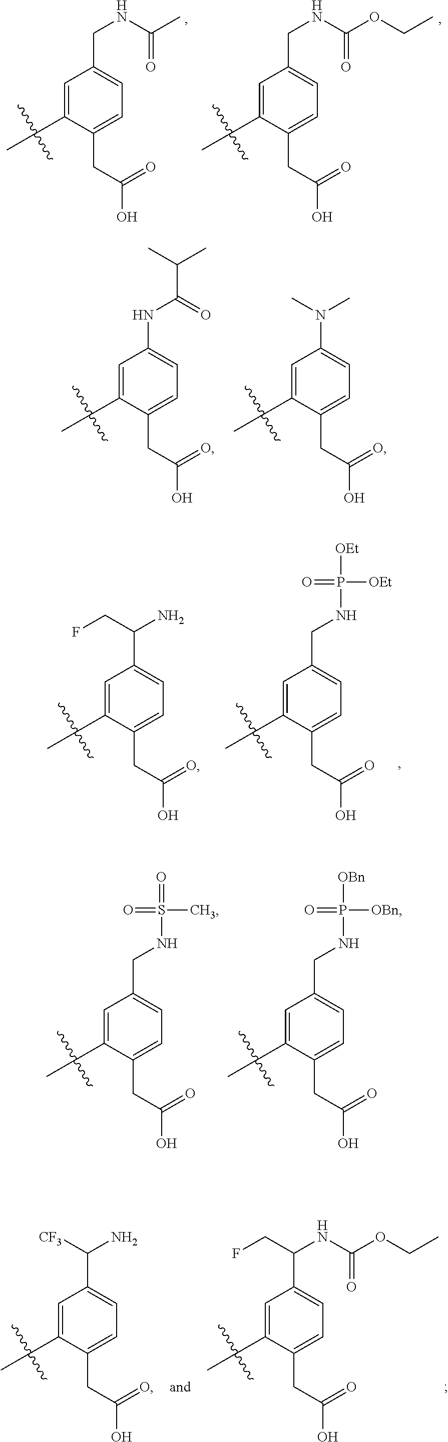
\textcircled{B} is selected from the group consisting of 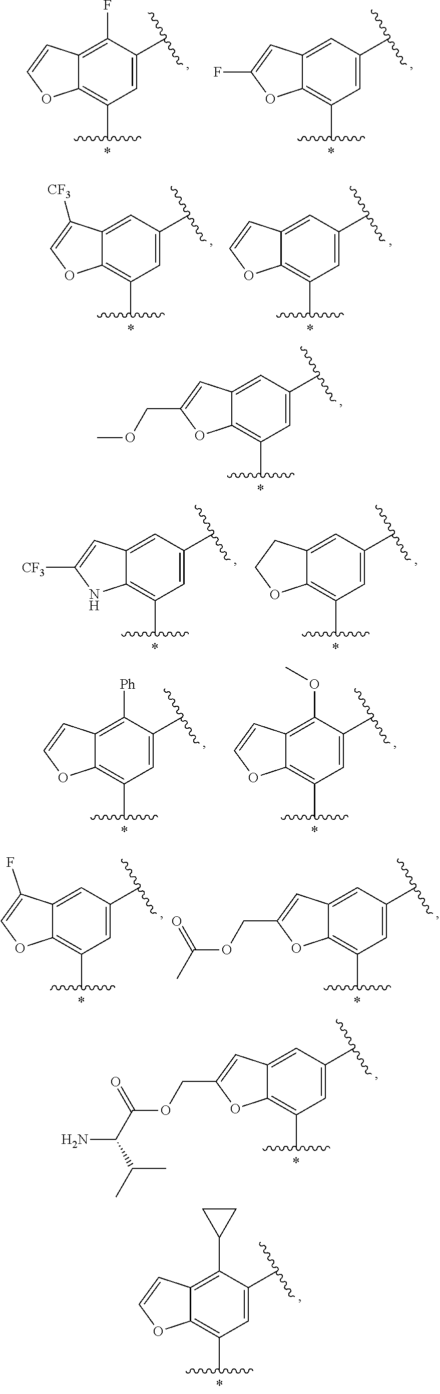 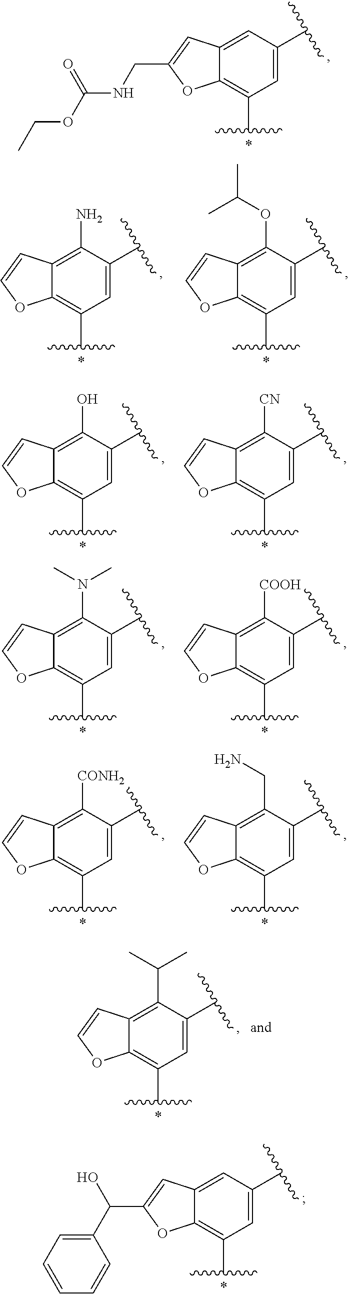
wherein the asterisk (*) indicates the point of attachment to \textcircled{C}; 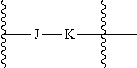
is selected from the group consisting of 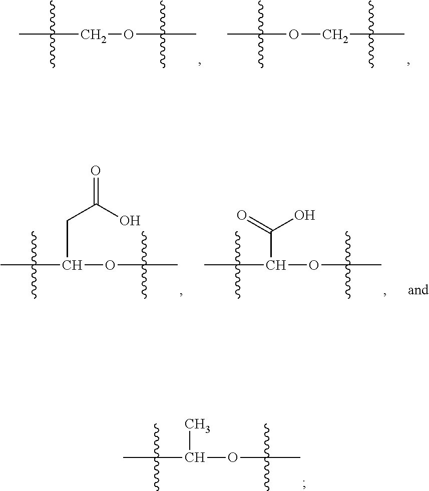
and
\textcircled{C} is selected from the group consisting of

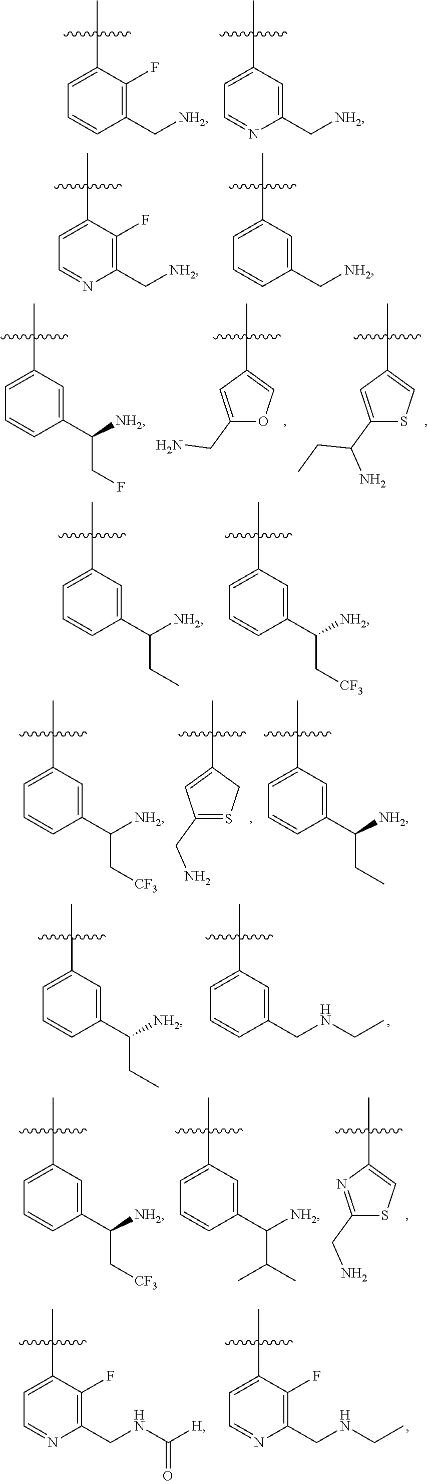

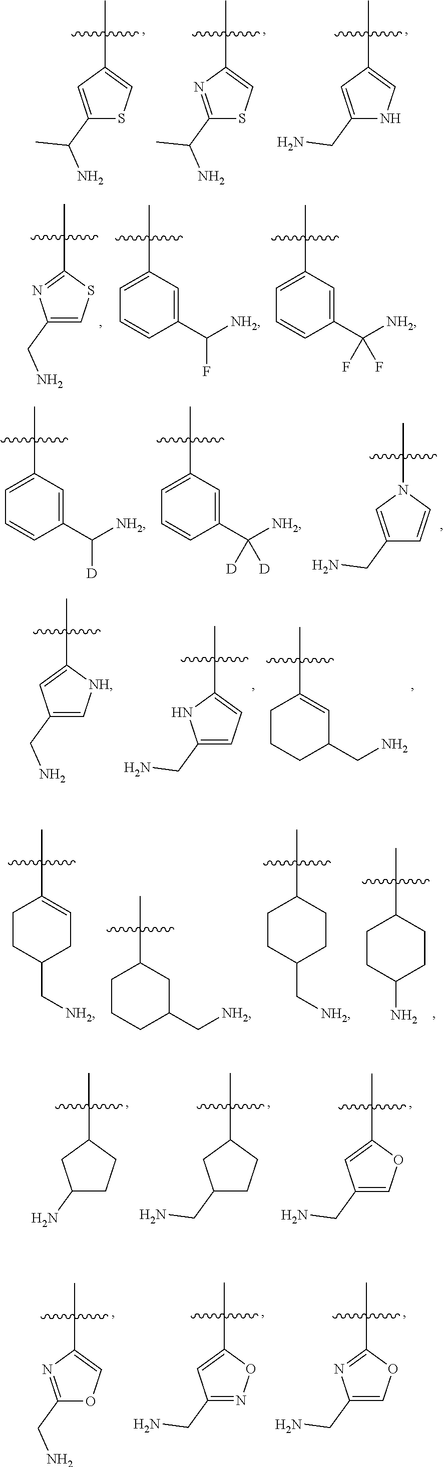

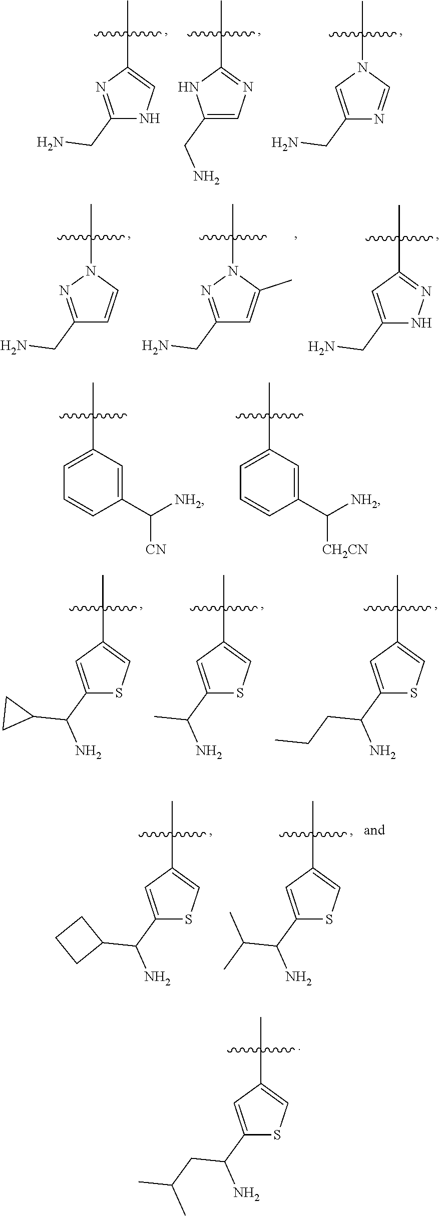

2. The compound of claim 1, wherein \textcircled{B} is

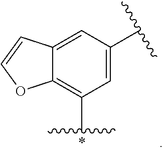 [SMILES: *c1cc(*)c2occc2c1]

3. The compound of claim 1, wherein 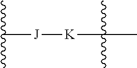
is

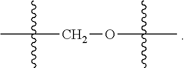 [SMILES: *CO*]

4. The compound of claim 1, wherein \textcircled{C} is selected from the group consisting of

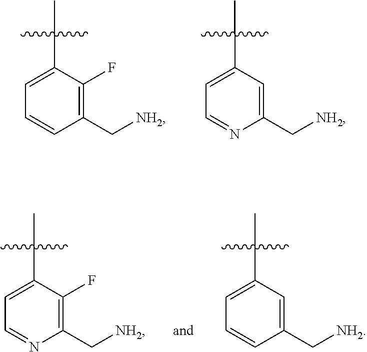 [SMILES: *c1cccc(CN)c1F ; *c1ccnc(CN)c1 ; *c1ccnc(CN)c1F ; *c1cccc(CN)c1]

5. The compound of claim 4, wherein \textcircled{C} is

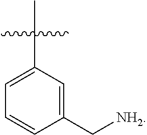 [SMILES: *c1cccc(CN)c1]

6. A compound of formula (II), or a pharmaceutically acceptable salt thereof: 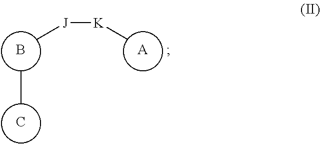
wherein:
\textcircled{A} is selected from the group consisting of 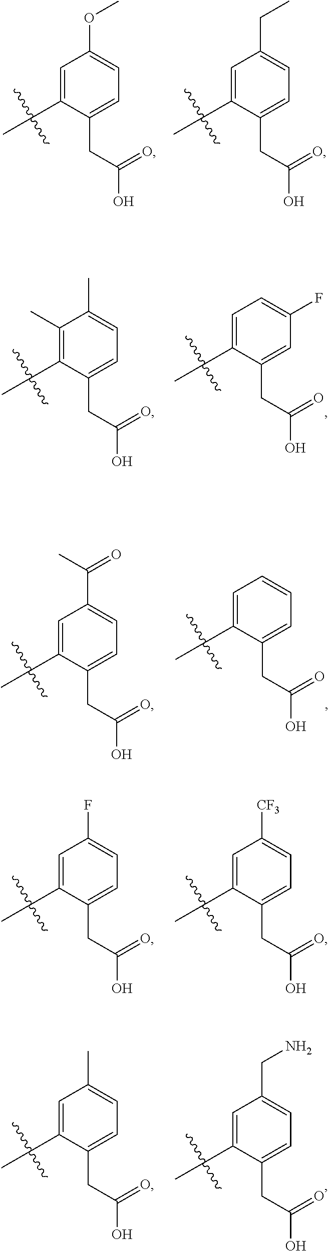 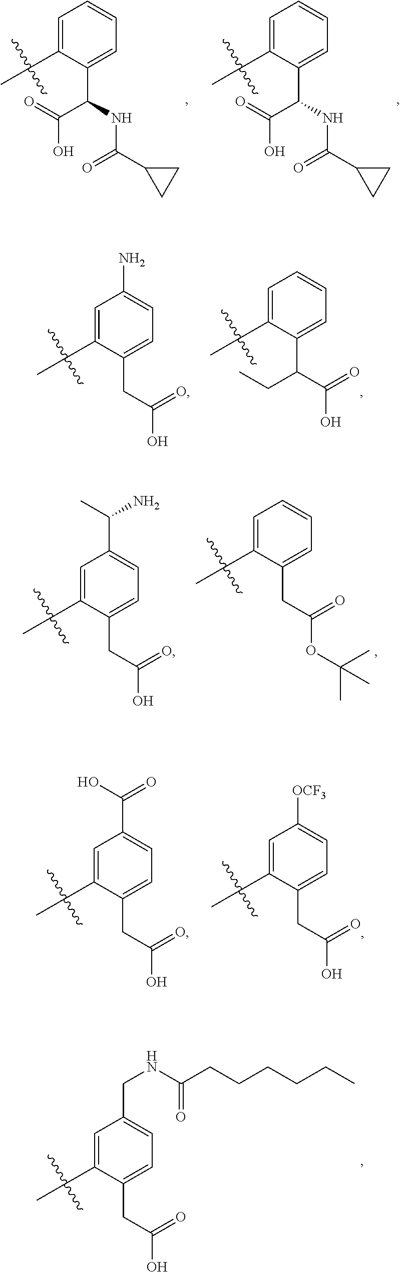 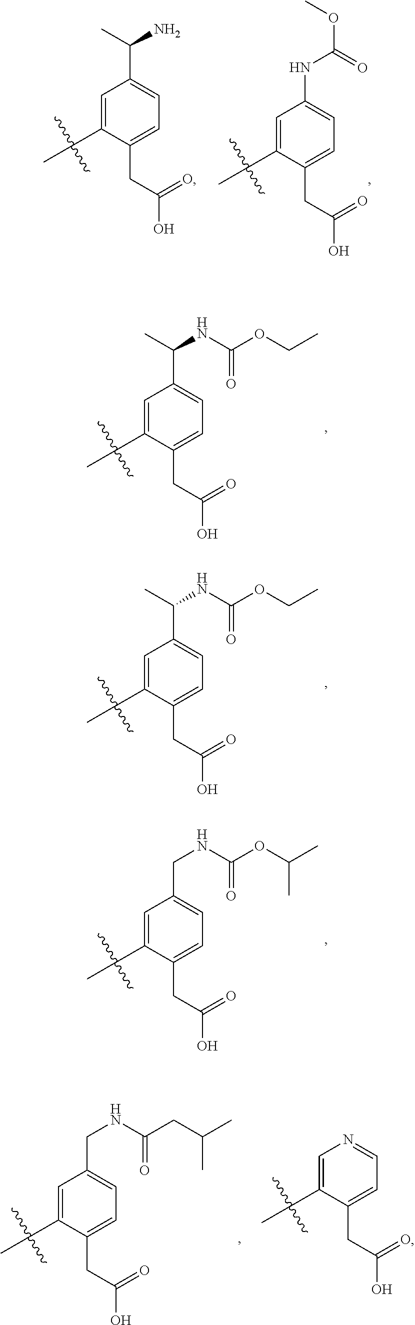 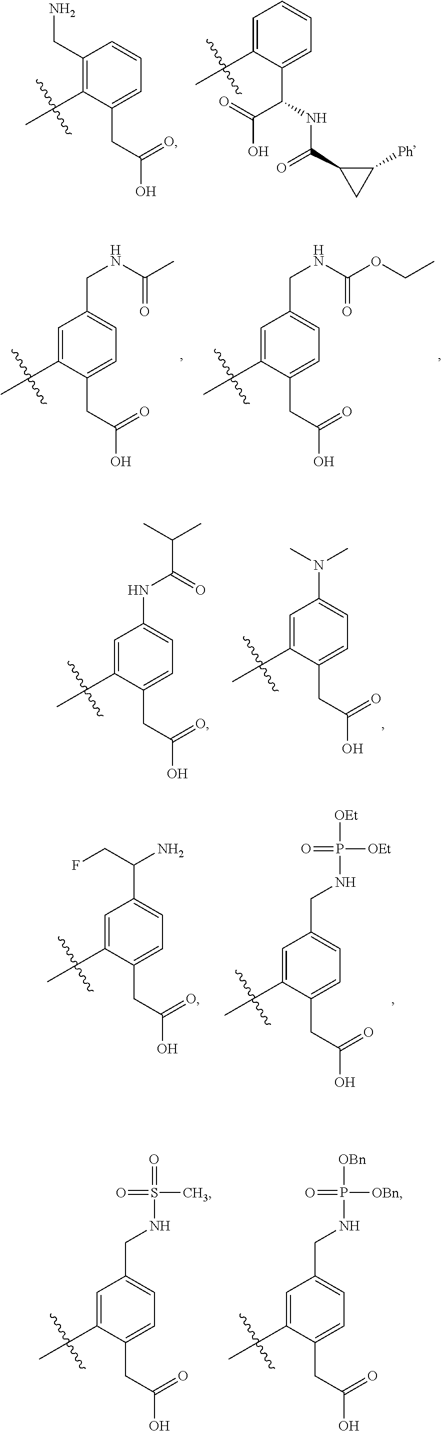 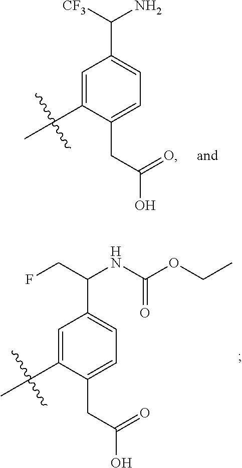 [SMILES: *c1cc(C(N)C(F)(F)F)ccc1CC(=O)O ; *c1cc(C(CF)NC(=O)OCC)ccc1CC(=O)O]
\textcircled{B} is selected from the group consisting of 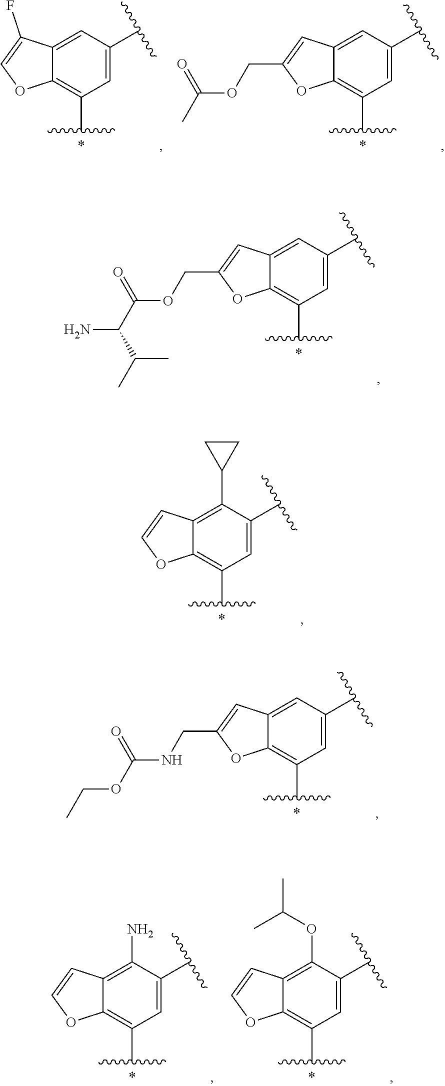 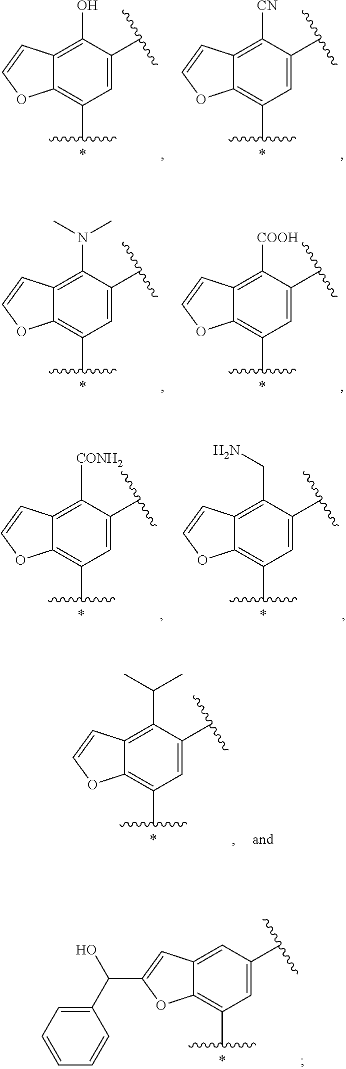
wherein the asterisk (*) indicates the point of attachment to \textcircled{C}; 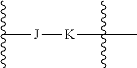
is selected from the group consisting of 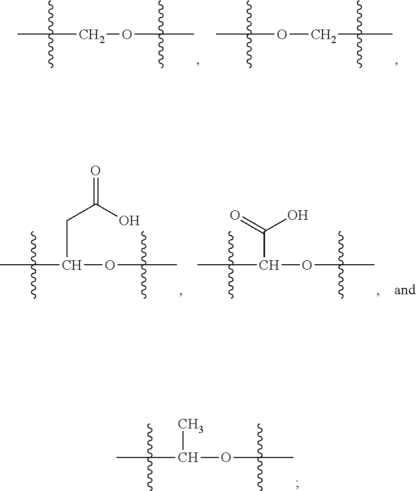
and
\textcircled{C} is selected from the group consisting of

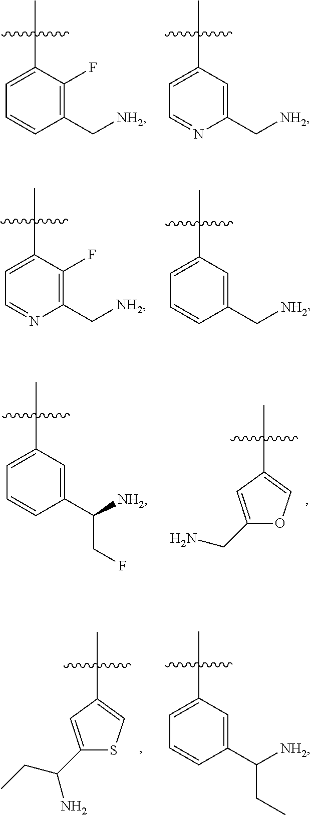

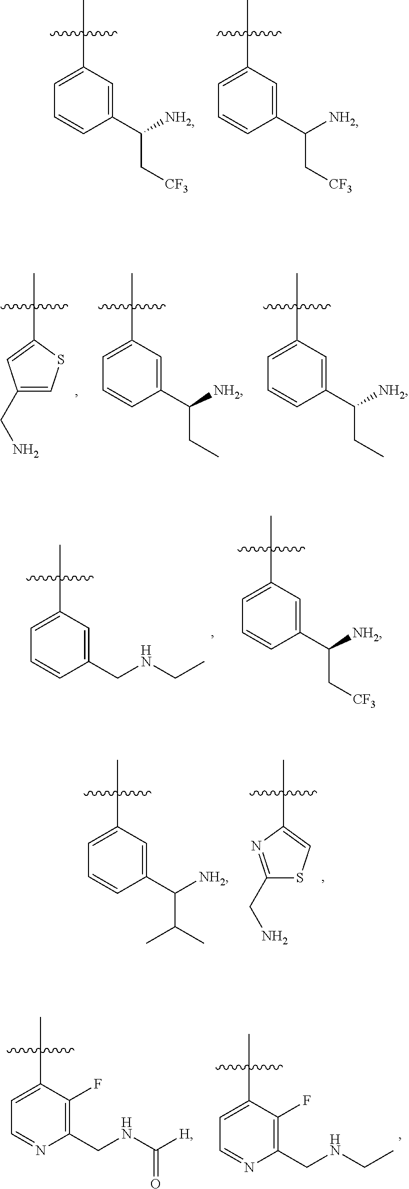

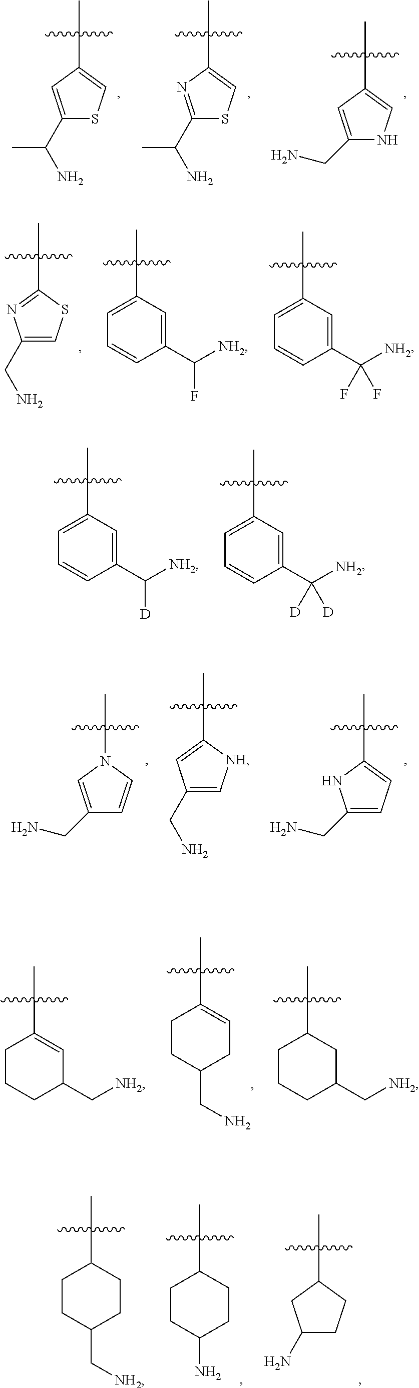

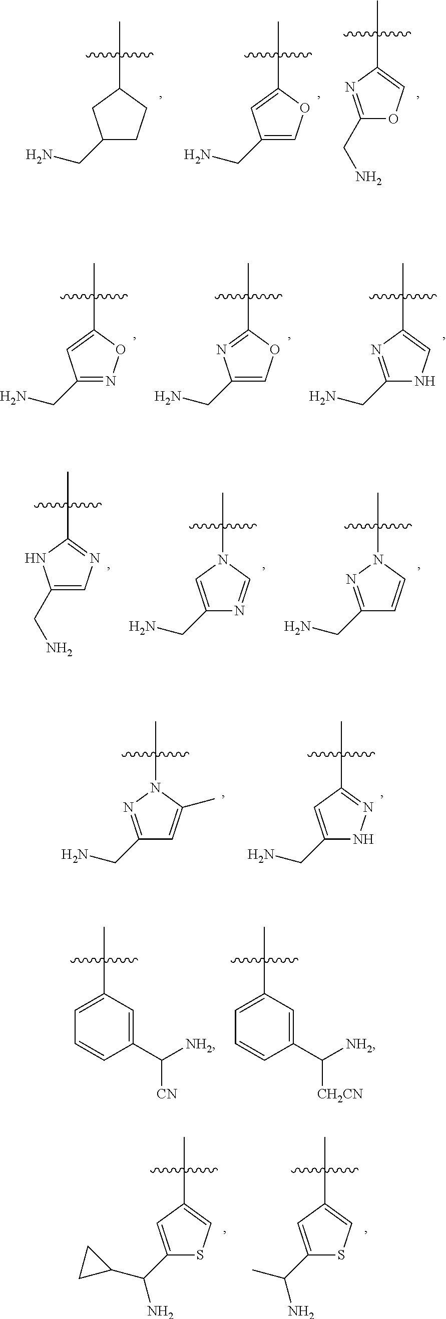

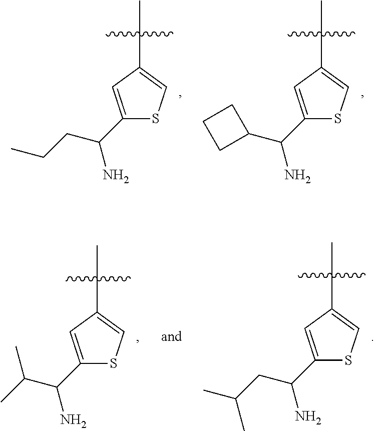 [SMILES: *c1csc(C(N)CCC)c1 ; *c1csc(C(N)C2CCC2)c1 ; *c1csc(C(N)C(C)C)c1 ; *c1csc(C(N)CC(C)C)c1]

7. The compound of claim 6, wherein \textcircled{A} is

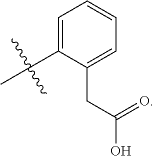 [SMILES: *c1ccccc1CC(=O)O]

8. The compound of claim 6, wherein 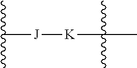
is

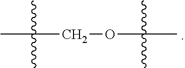 [SMILES: *CO*]

9. The compound of claim 6, wherein \textcircled{C} is selected from the group consisting of

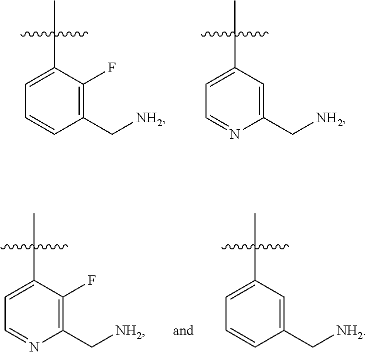 [SMILES: *c1cccc(CN)c1F ; *c1ccnc(CN)c1 ; *c1ccnc(CN)c1F ; *c1cccc(CN)c1]

10. The compound of claim 9, wherein \textcircled{C} is

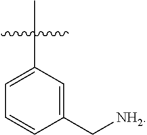 [SMILES: *c1cccc(CN)c1]

11. A compound of formula (III), or a pharmaceutically acceptable salt thereof: 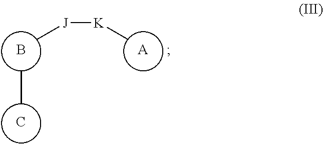
wherein:
\textcircled{A} is selected from the group consisting of 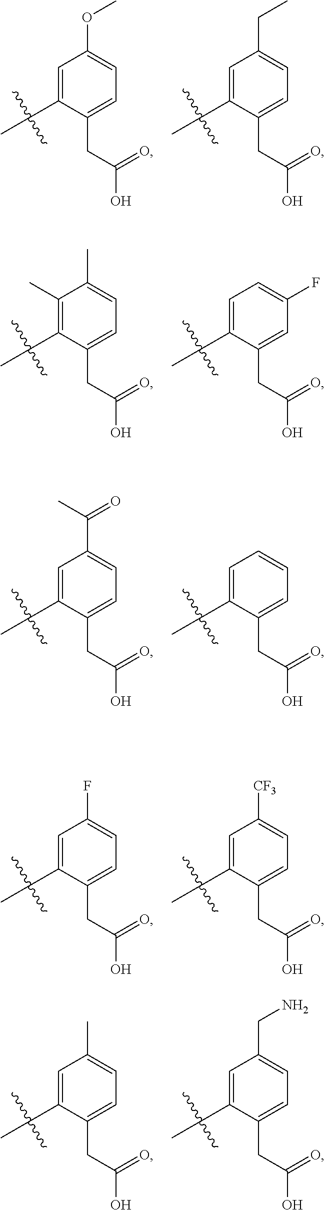 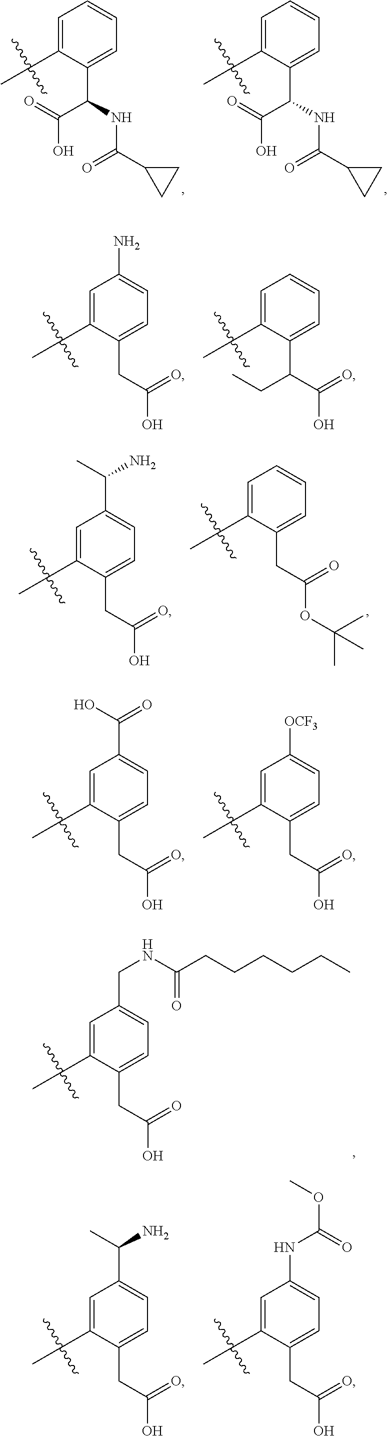 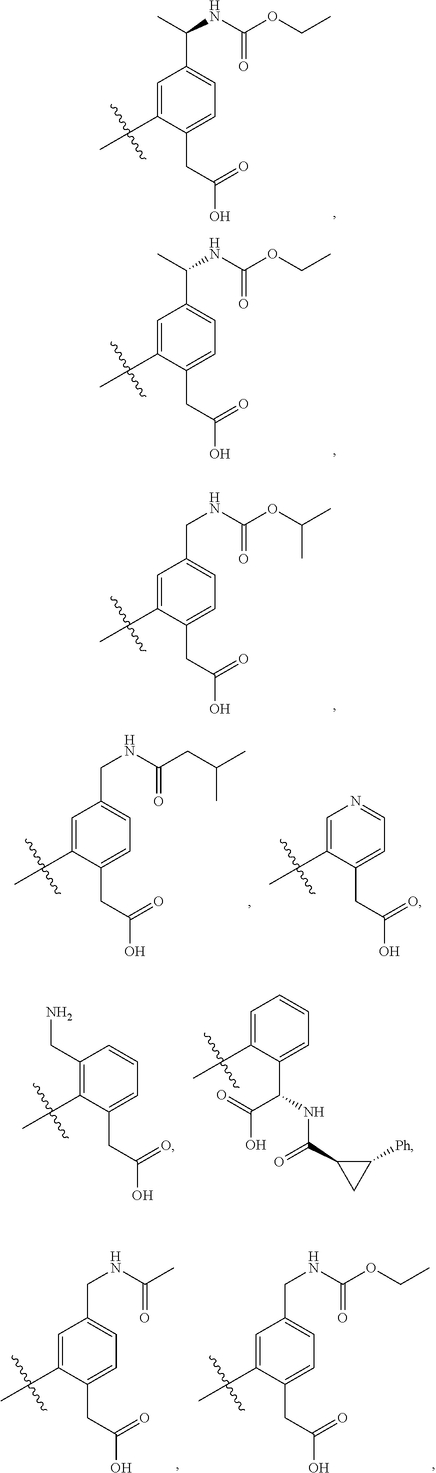 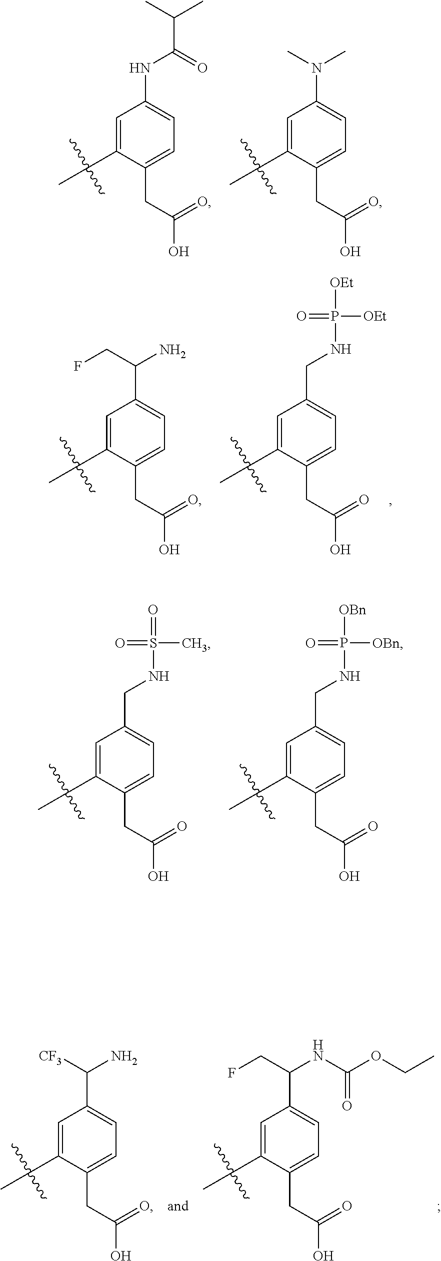
\textcircled{B} is selected from the group consisting of 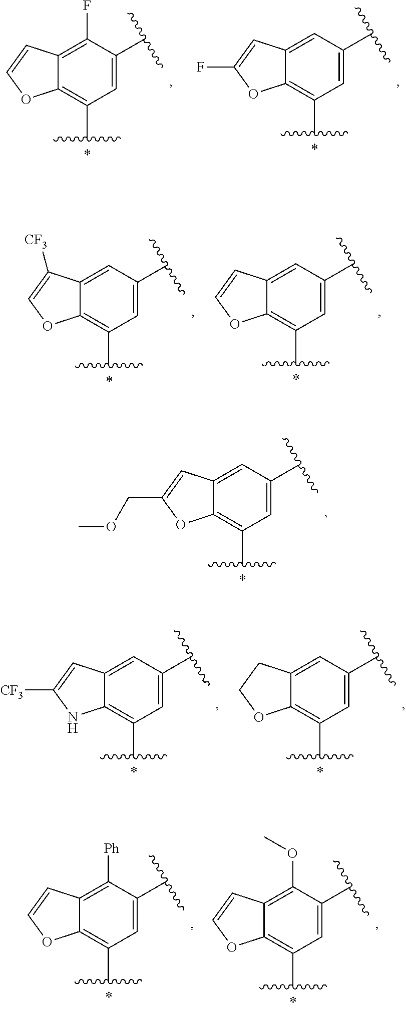 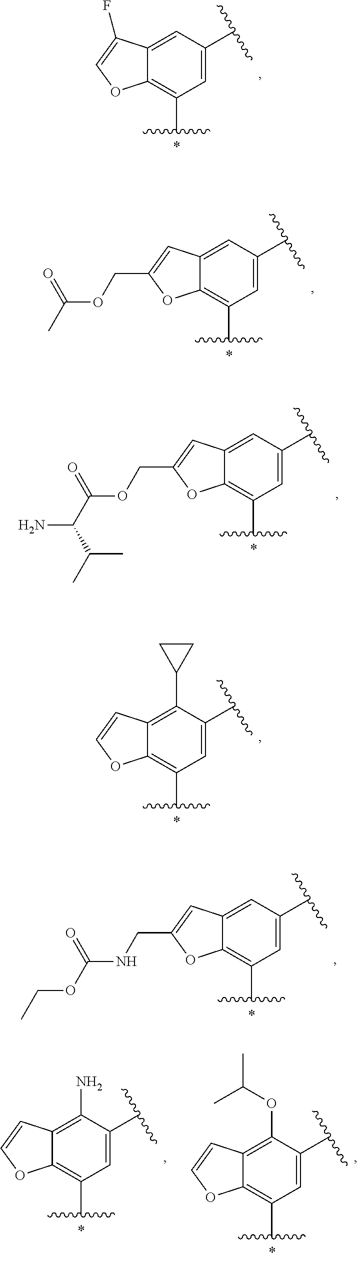 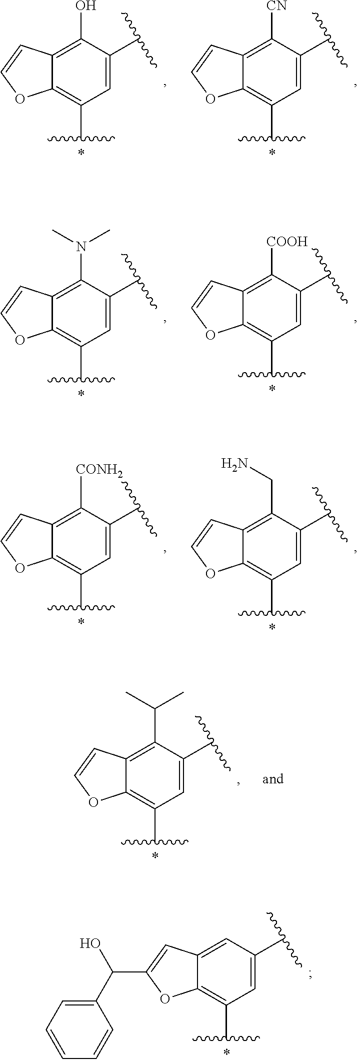
wherein the asterisk (*) indicates the point of attachment to \textcircled{C}; 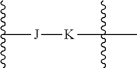
is selected from the group consisting of 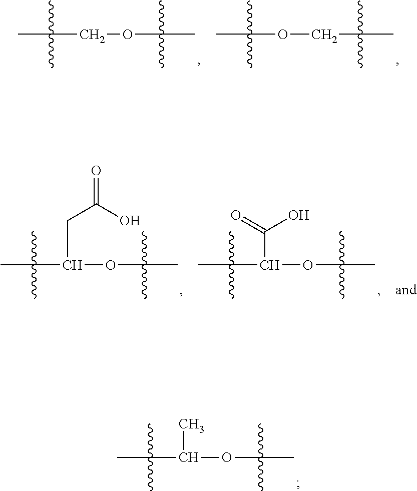
\textcircled{C} is selected from the group consisting of

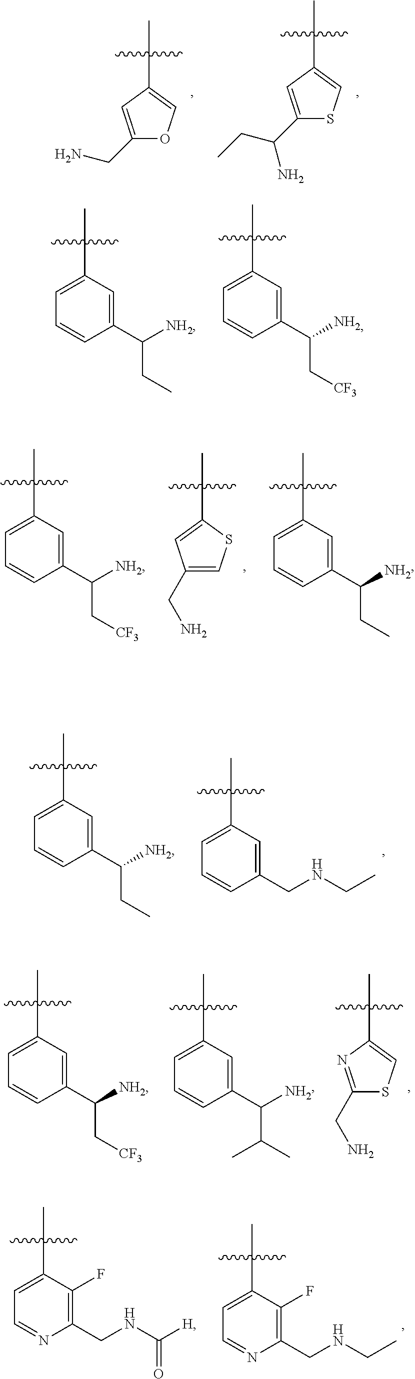

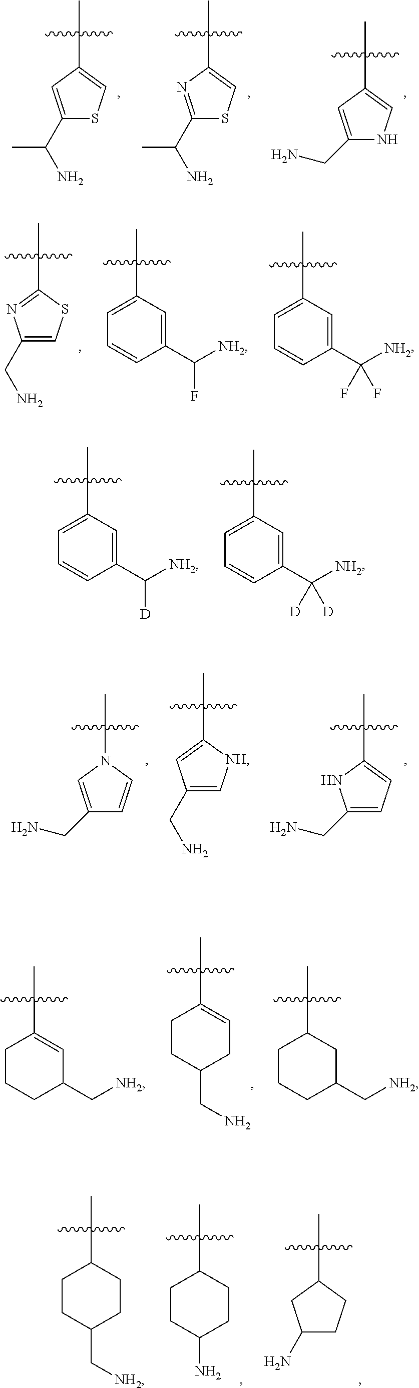

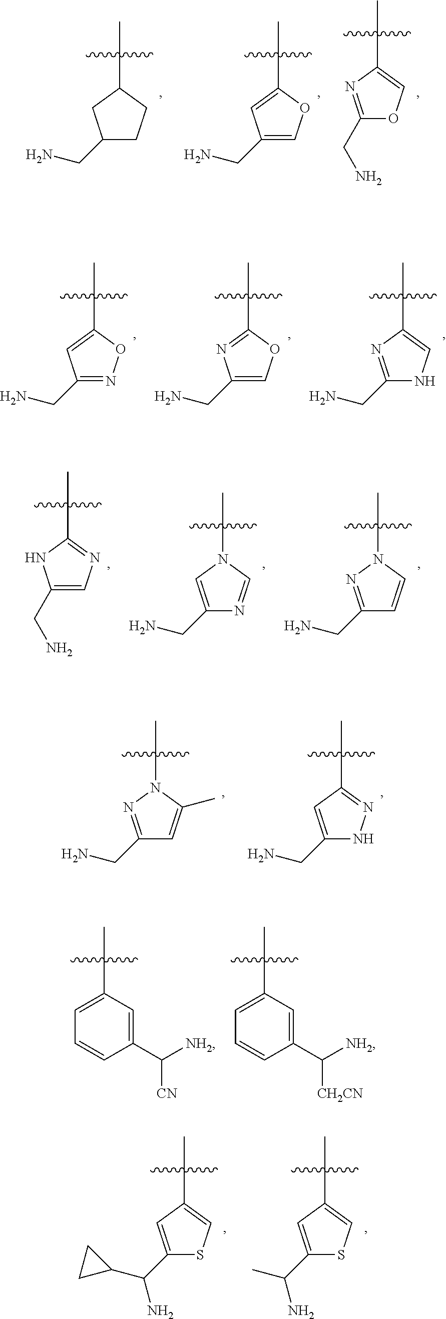

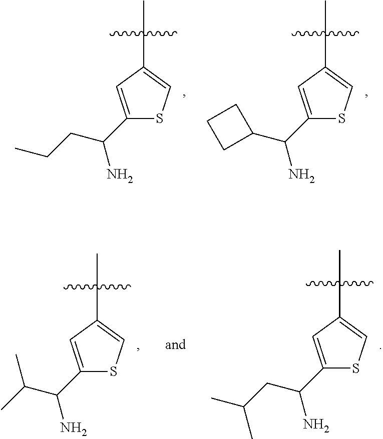 [SMILES: *c1csc(C(N)CCC)c1 ; *c1csc(C(N)C2CCC2)c1 ; *c1csc(C(N)C(C)C)c1 ; *c1csc(C(N)CC(C)C)c1]

12. The compound of claim 11, wherein \textcircled{A} is

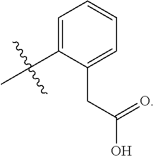 [SMILES: *c1ccccc1CC(=O)O]

13. The compound of claim 11, wherein \textcircled{B} is

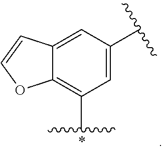 [SMILES: *c1cc(*)c2occc2c1]

14. The compound of claim 11, wherein 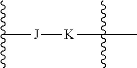
is

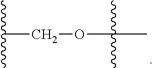 [SMILES: *CO*]

15. A compound of formula (IV), or a pharmaceutically acceptable salt thereof: 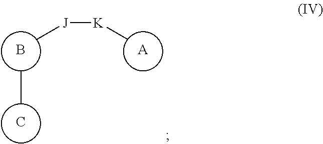
wherein:
\textcircled{A} is selected from the group consisting of 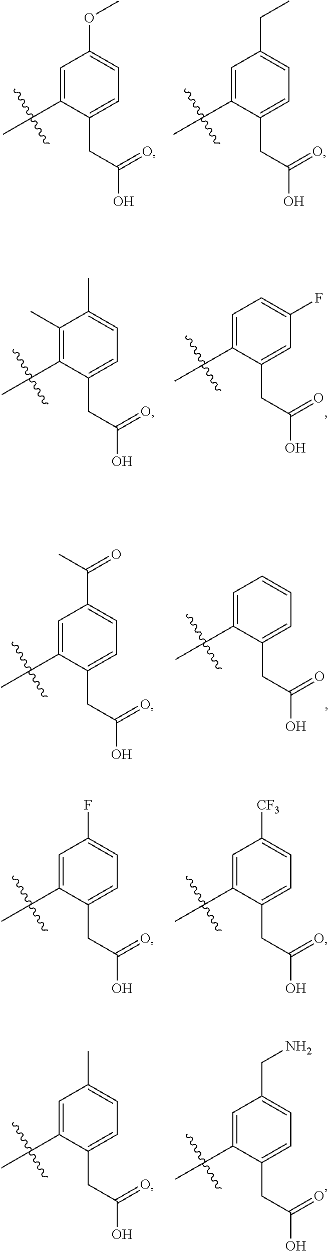 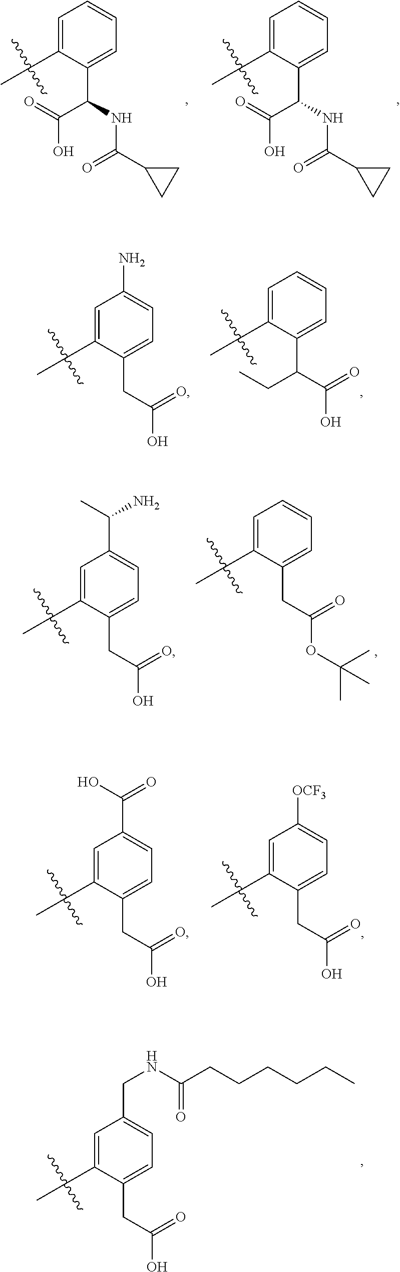 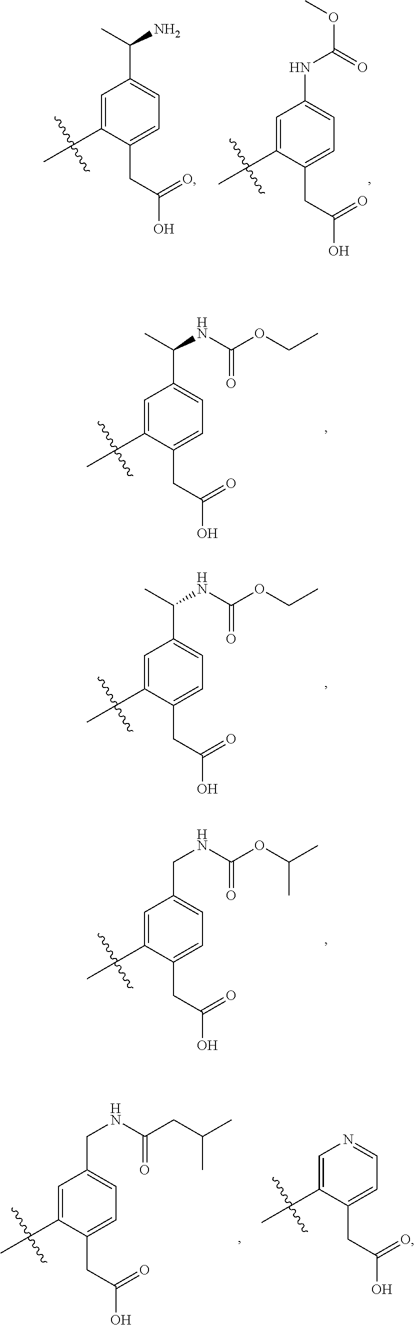 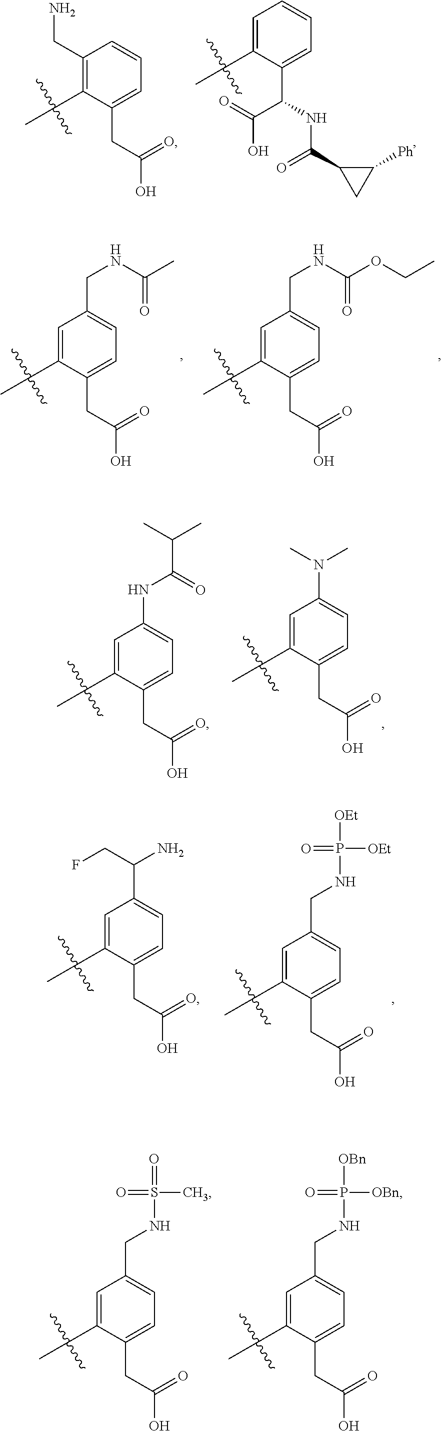 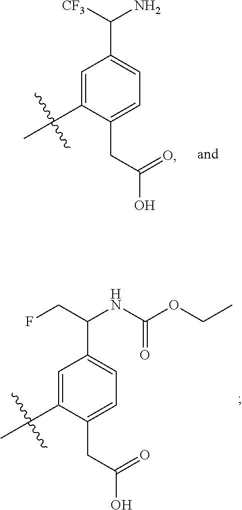 [SMILES: *c1cc(C(N)C(F)(F)F)ccc1CC(=O)O ; *c1cc(C(CF)NC(=O)OCC)ccc1CC(=O)O]
\textcircled{B} is selected from the group consisting of 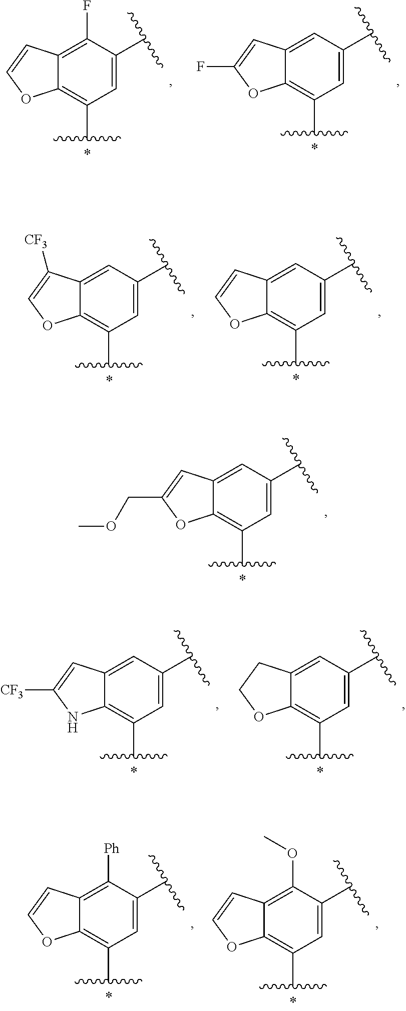 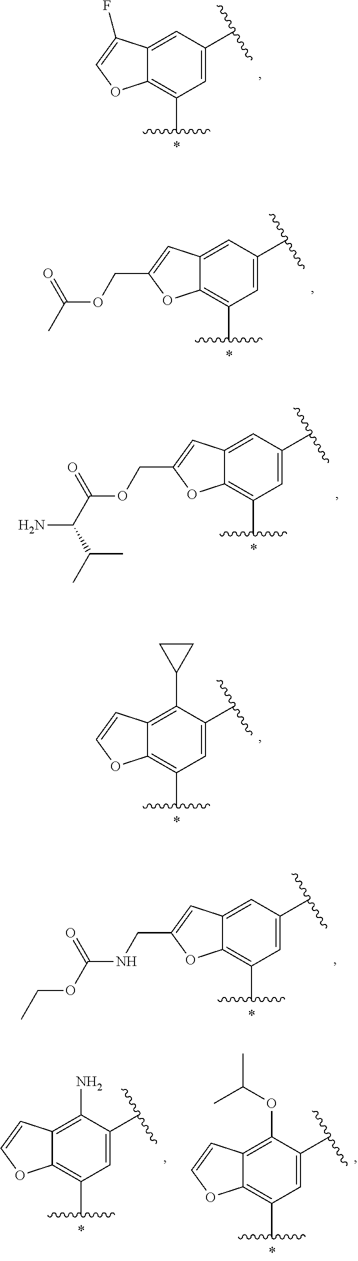 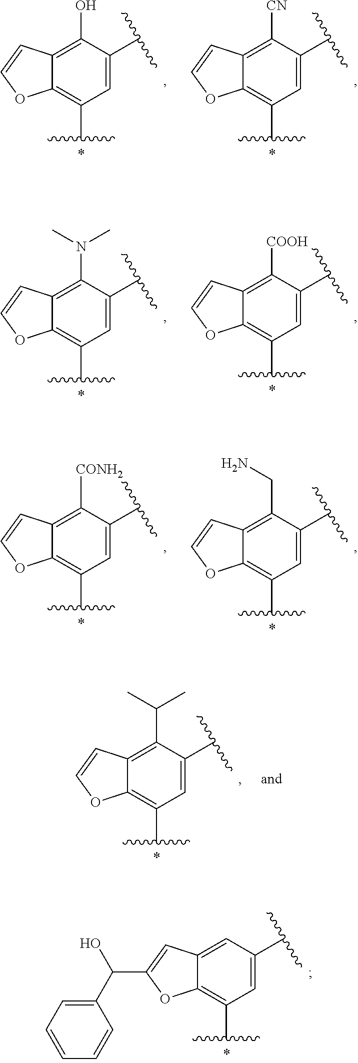
wherein the asterisk (*) indicates the point of attachment to \textcircled{C}; 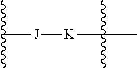
is selected from the group consisting of 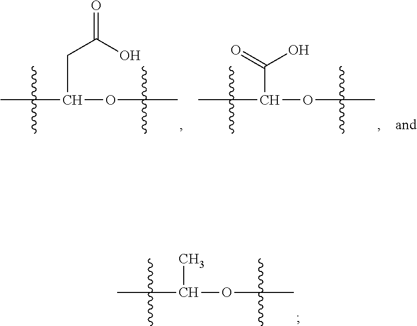
and
\textcircled{C} is selected from the group consisting of

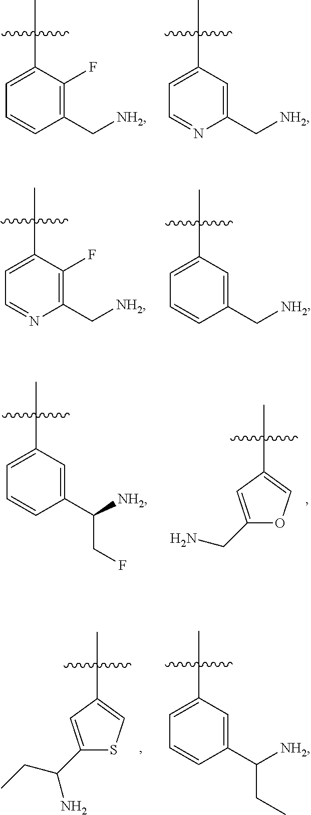

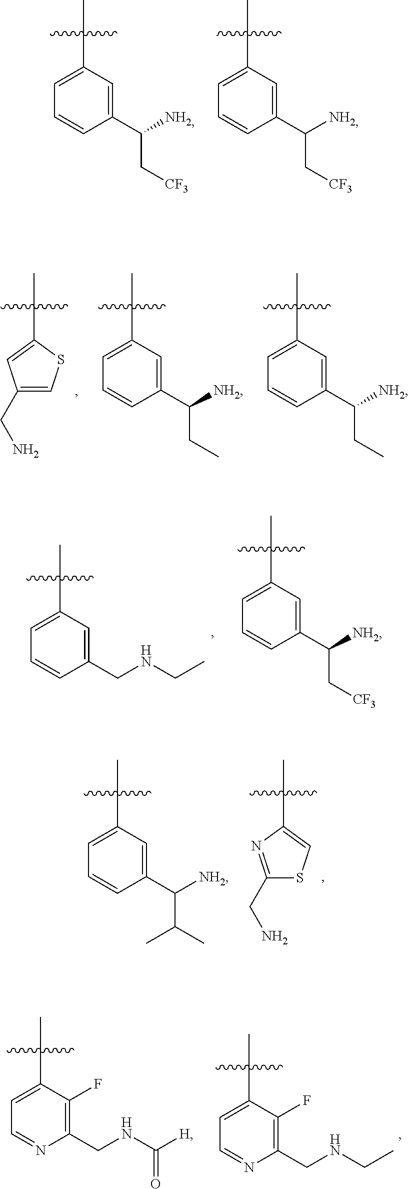

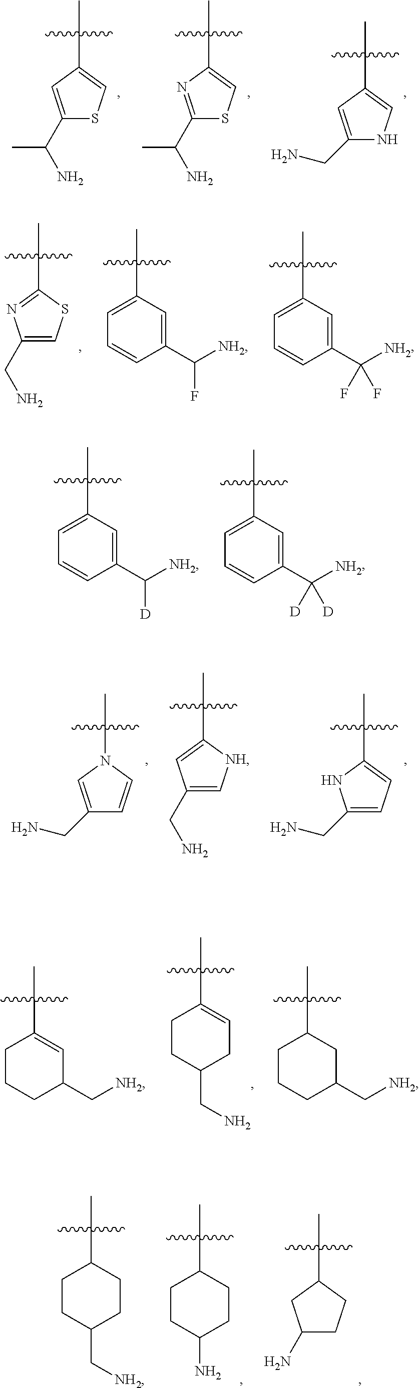

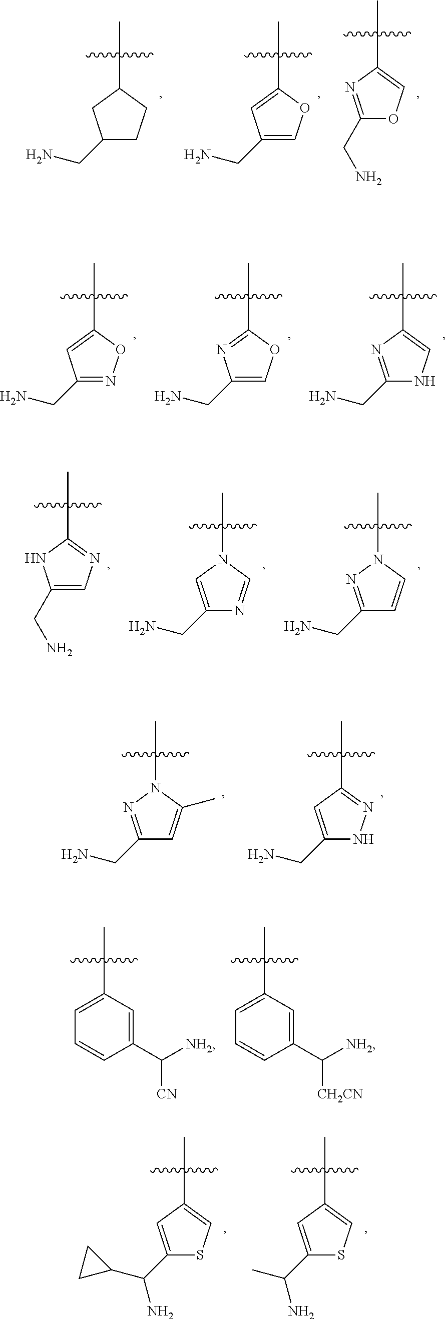

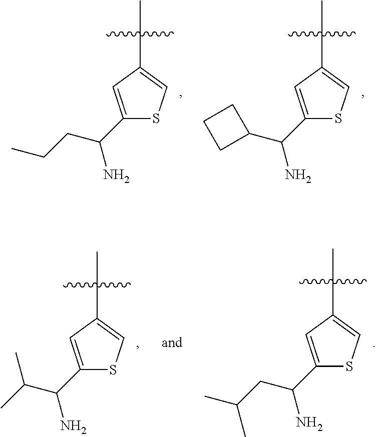 [SMILES: *c1csc(C(N)CCC)c1 ; *c1csc(C(N)C2CCC2)c1 ; *c1csc(C(N)C(C)C)c1 ; *c1csc(C(N)CC(C)C)c1]

16. The compound of claim 15, wherein \textcircled{A} is

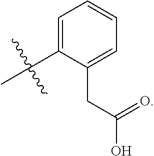 [SMILES: *c1ccccc1CC(=O)O]

17. The compound of claim 15, wherein \textcircled{B} is

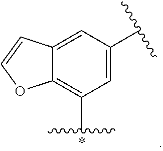 [SMILES: *c1cc(*)c2occc2c1]

18. The compound of claim 15, wherein \textcircled{C} is selected from the group consisting of

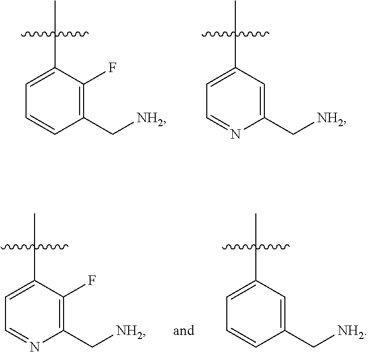 [SMILES: *c1cccc(CN)c1F ; *c1ccnc(CN)c1 ; *c1ccnc(CN)c1F ; *c1cccc(CN)c1]

19. The compound of claim 18, wherein \textcircled{C} is

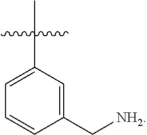 [SMILES: *c1cccc(CN)c1]

20. A compound, or a pharmaceutically acceptable salt thereof, selected from the following table: 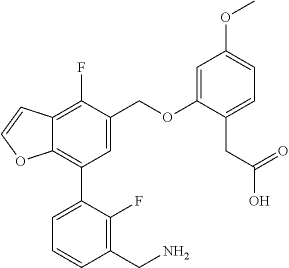 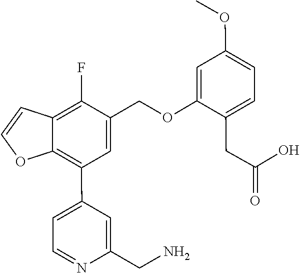 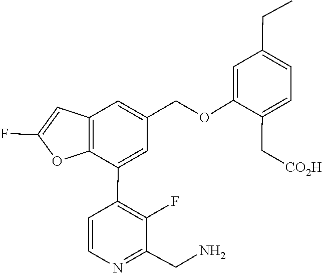 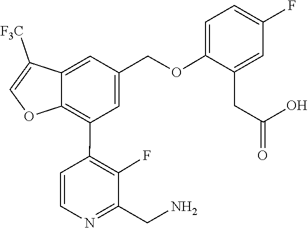 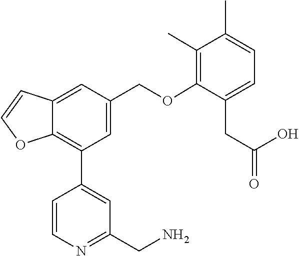 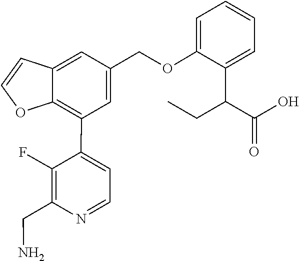 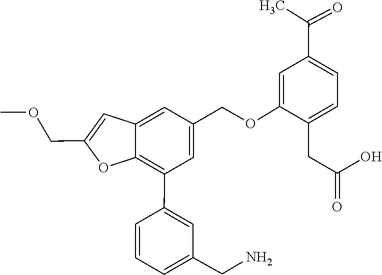 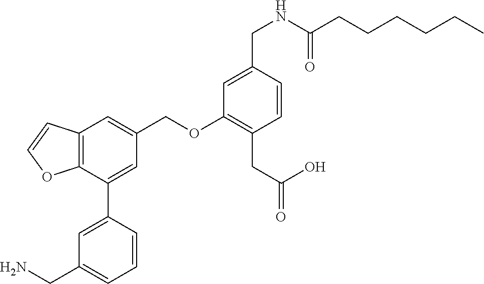 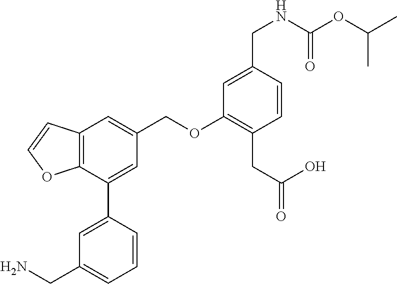 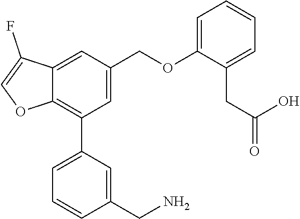 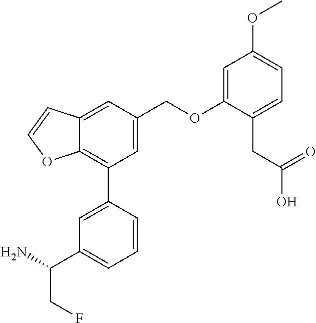 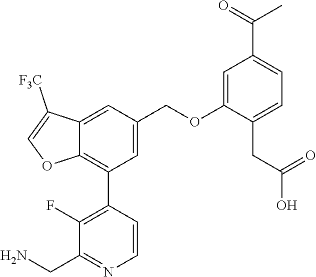 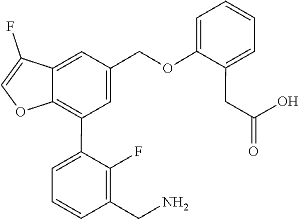 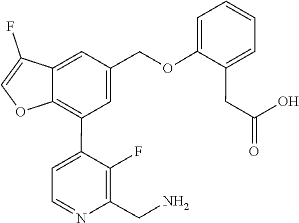 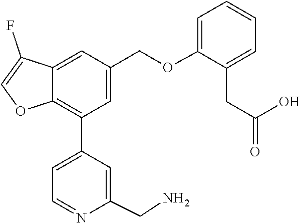 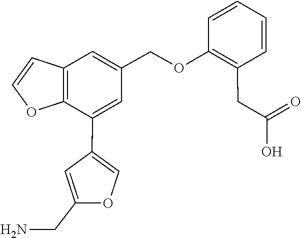 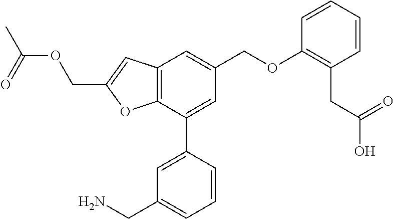 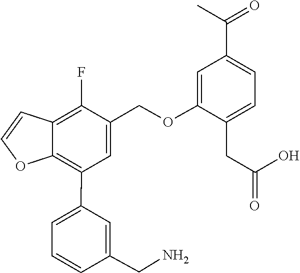 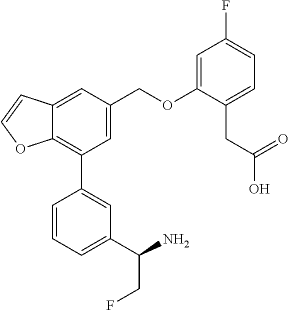 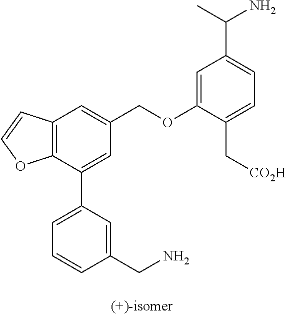 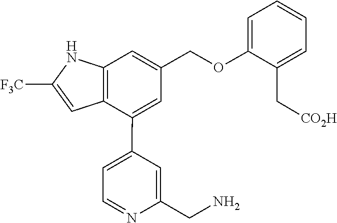 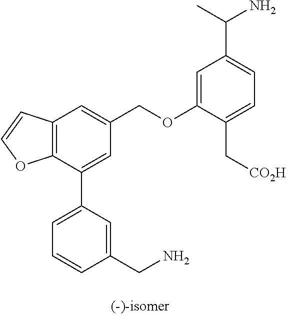 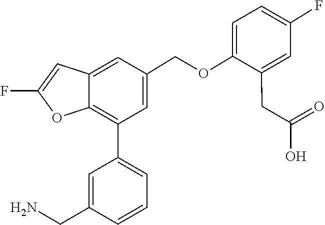 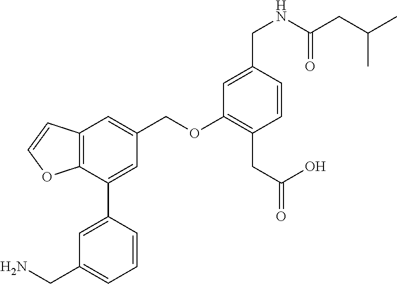 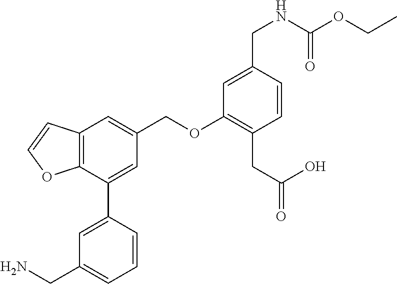 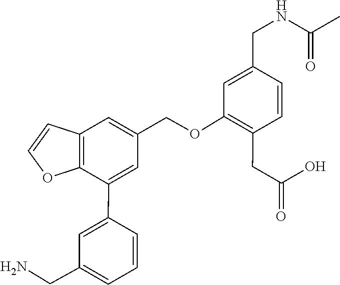 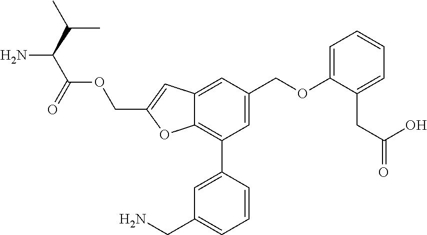 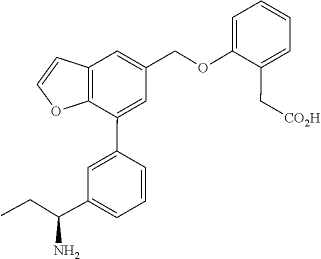 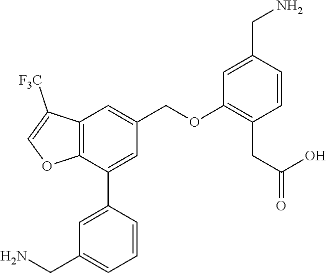 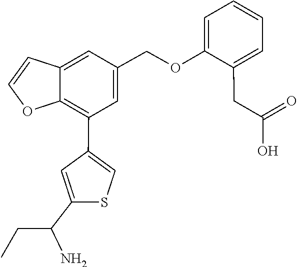 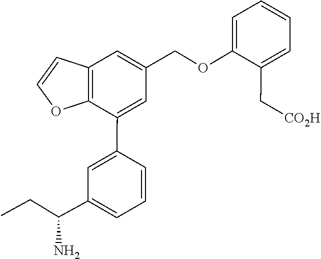 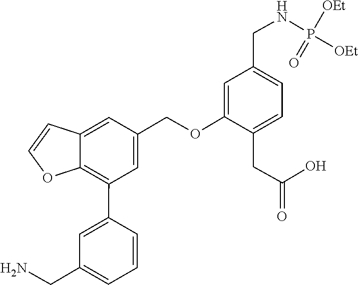 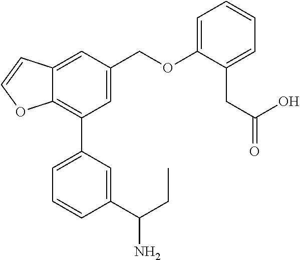 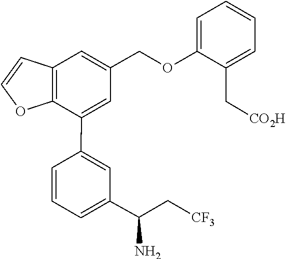 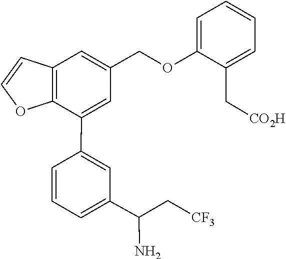 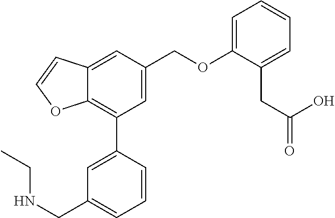 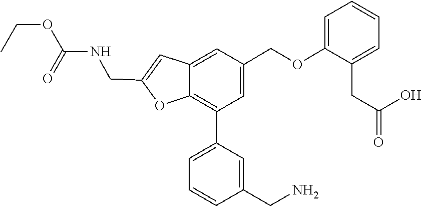 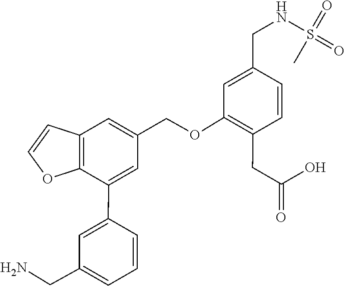 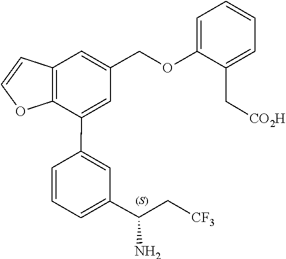 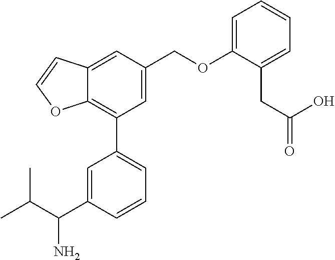 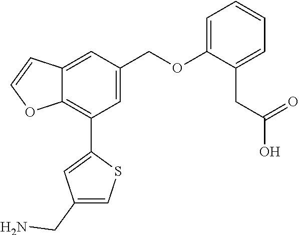 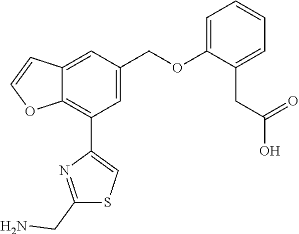 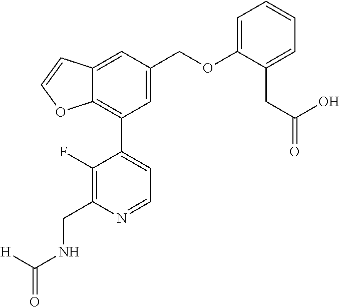 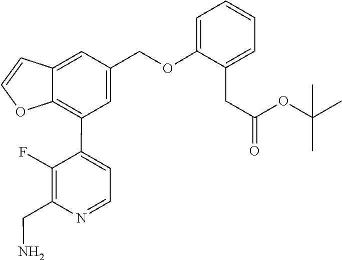 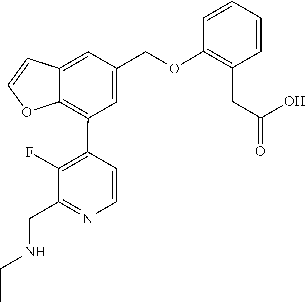 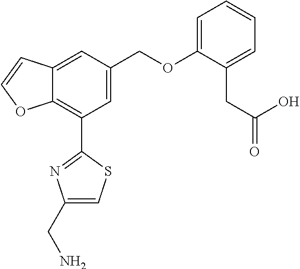 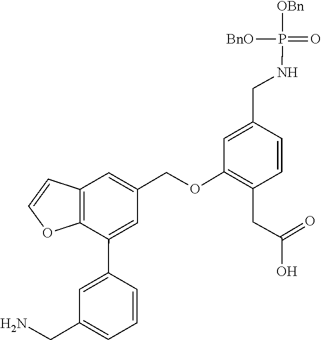 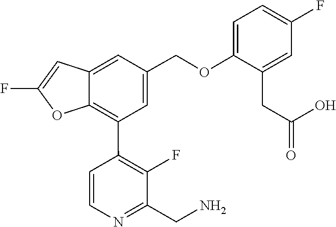 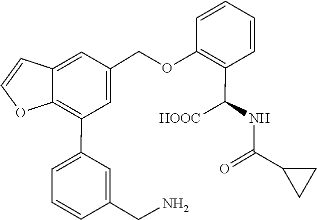 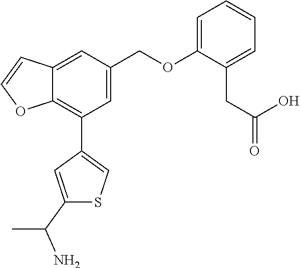 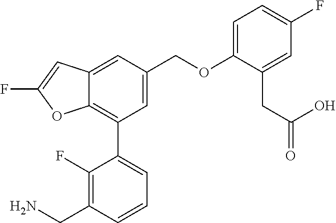 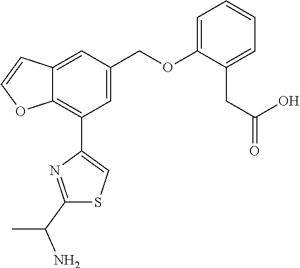 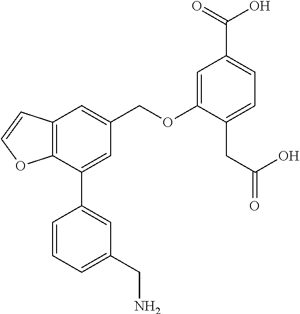 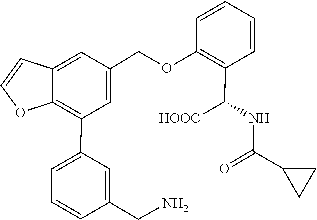 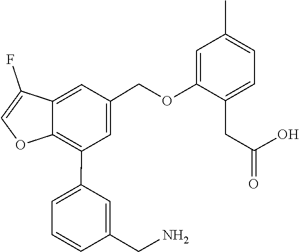 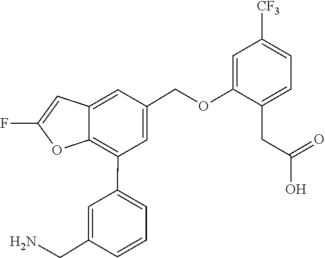 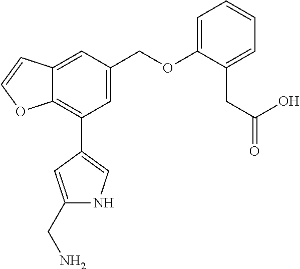 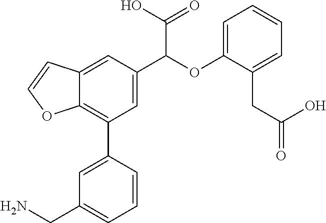 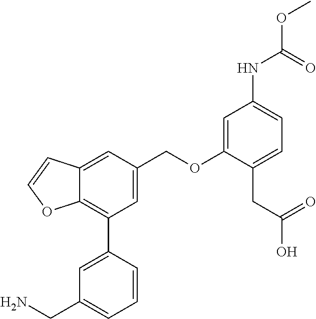 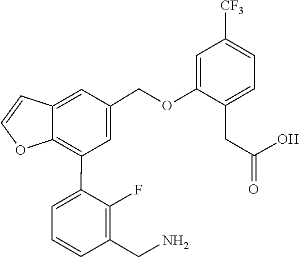 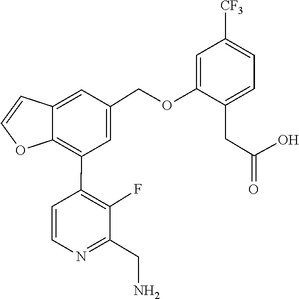 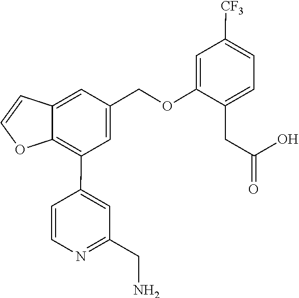 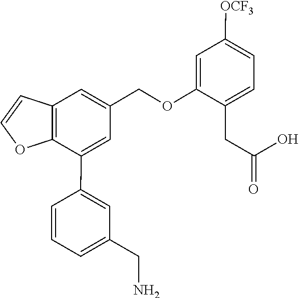 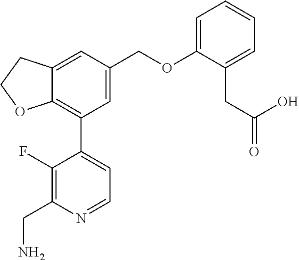 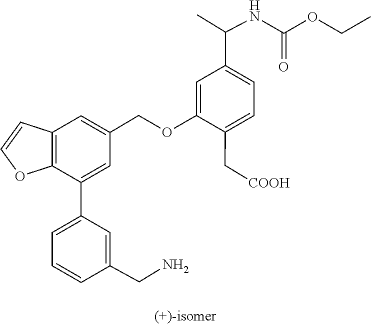 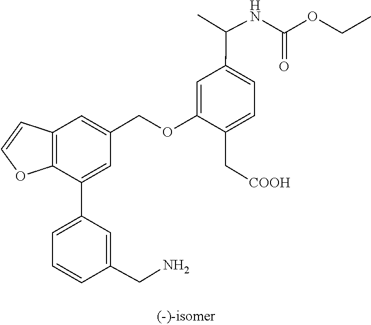 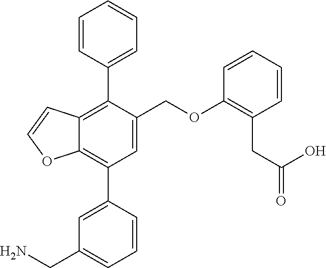 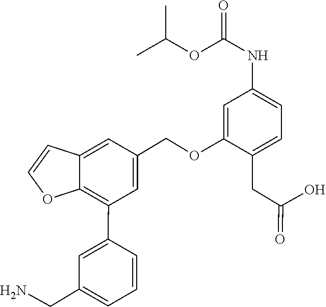 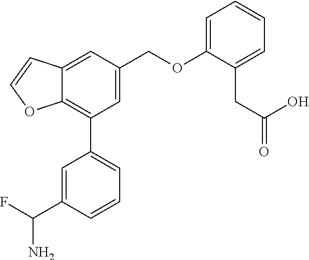 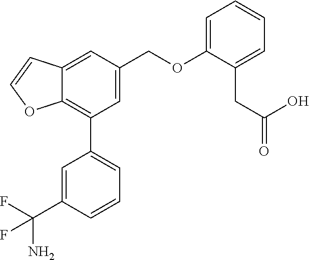 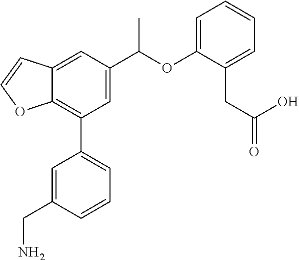 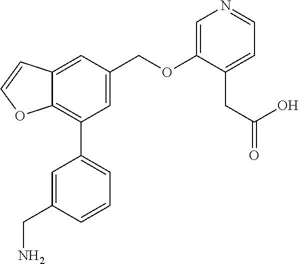 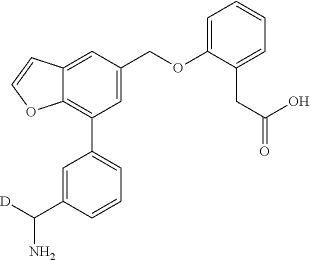 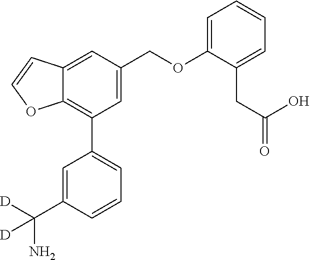 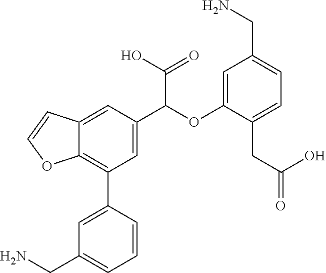 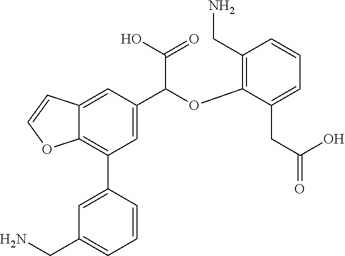 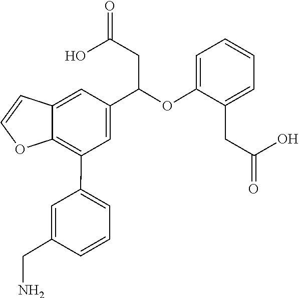 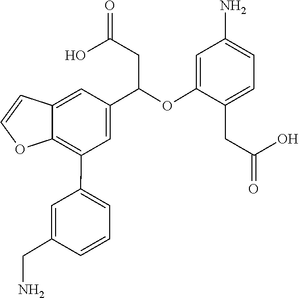 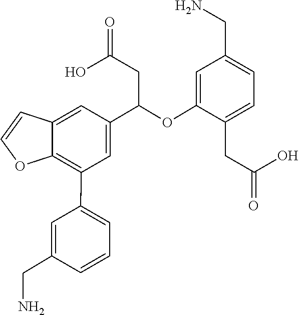 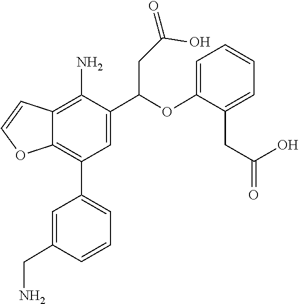 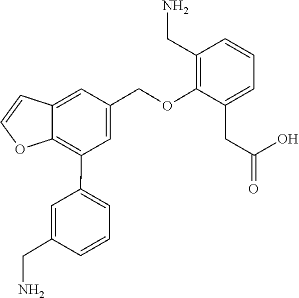 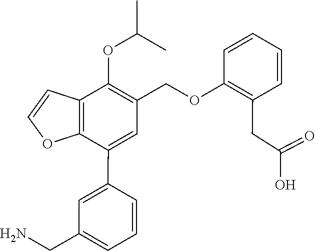 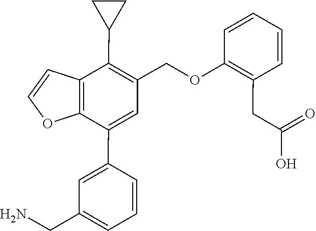 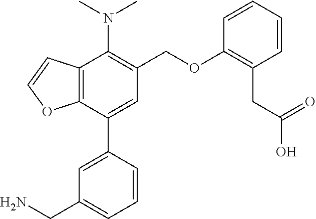 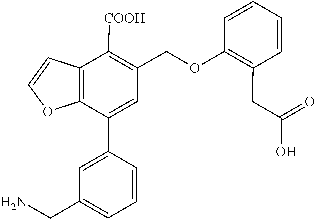 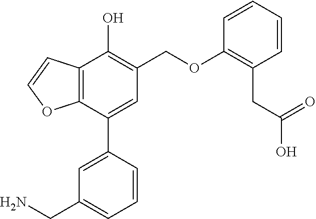 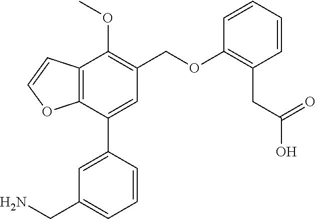 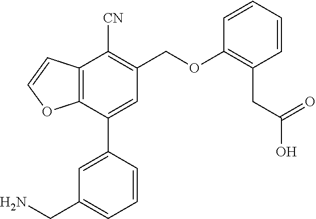 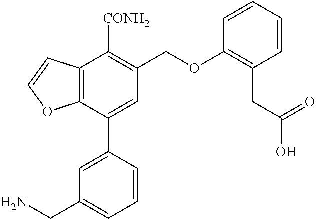 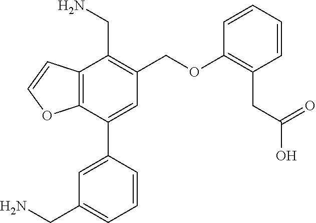 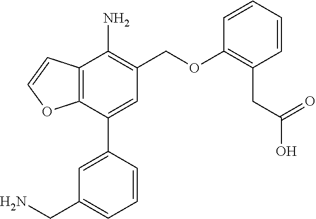 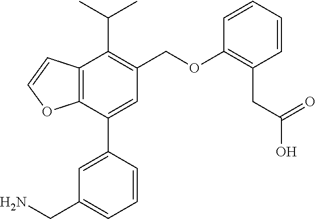 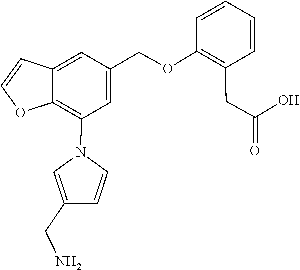 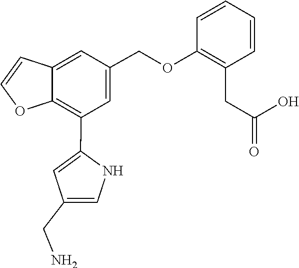 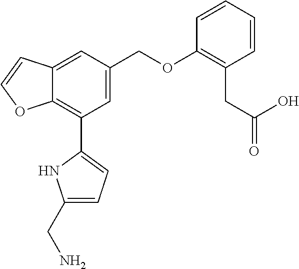 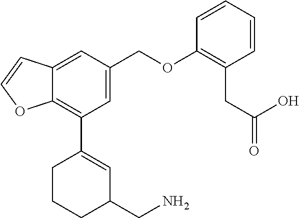 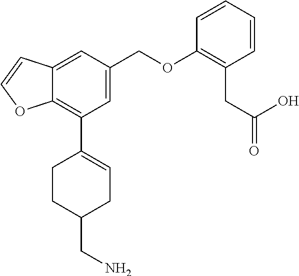 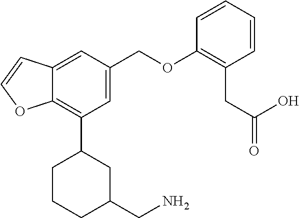 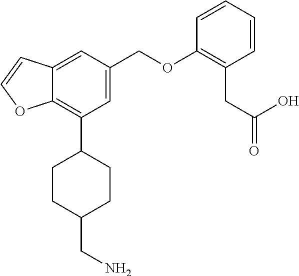 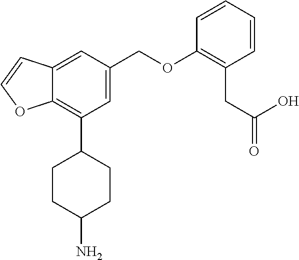 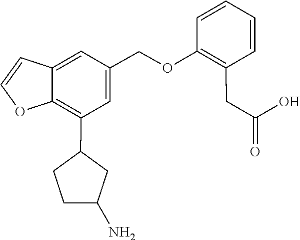 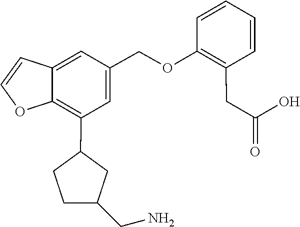 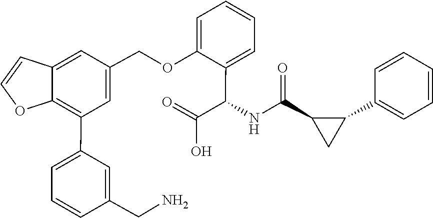 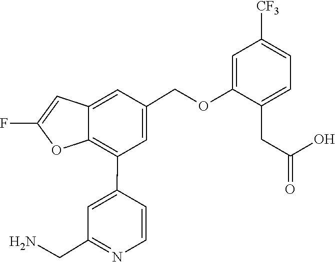 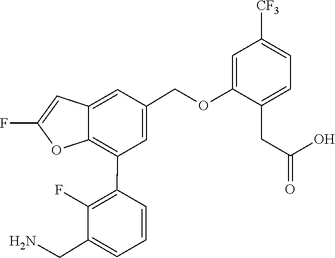 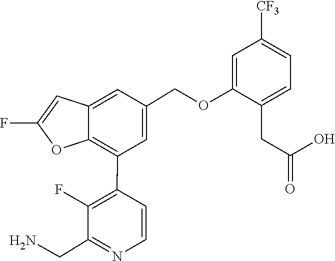 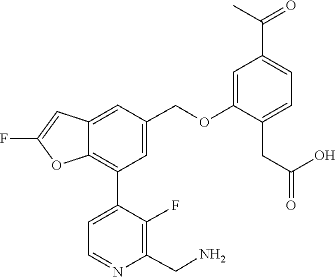 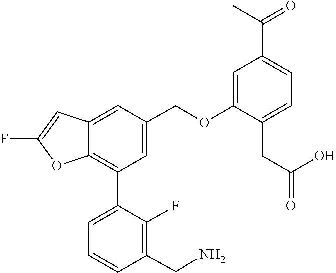 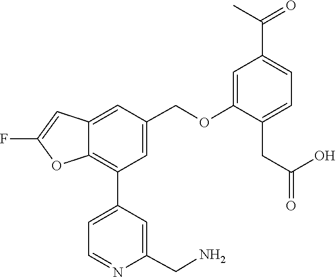 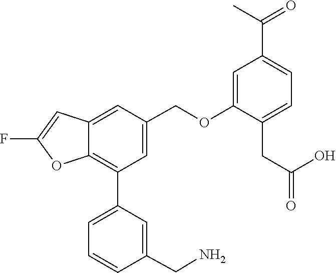 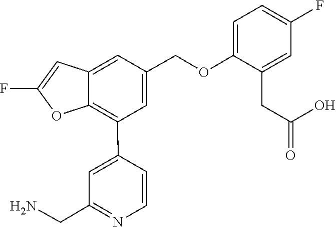 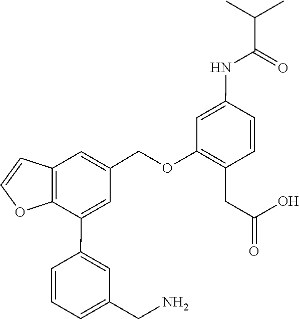 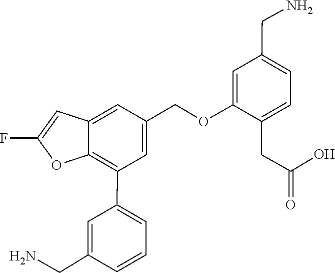 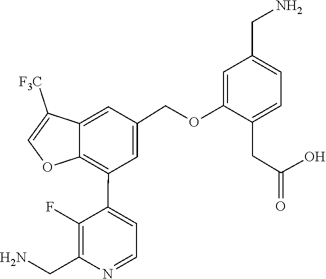 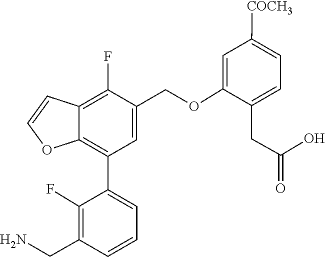 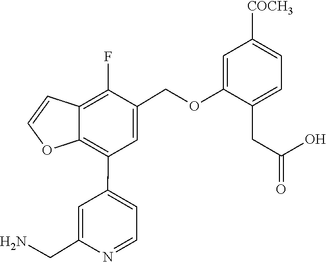 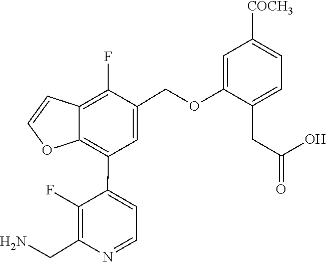 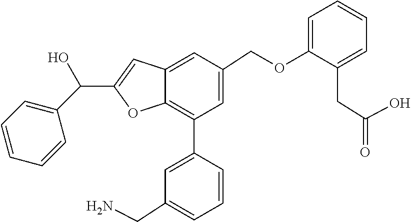 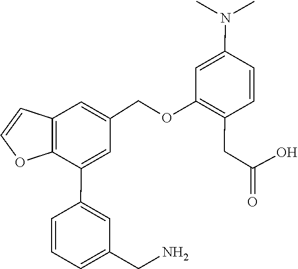 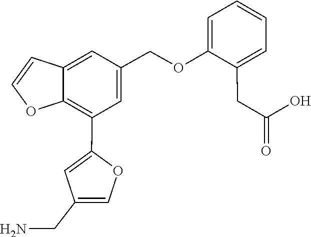 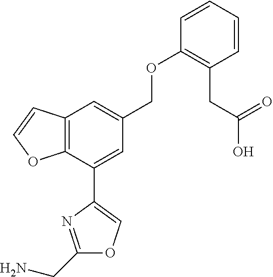 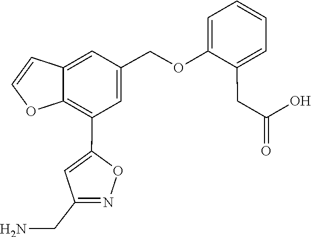 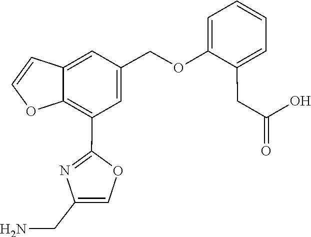 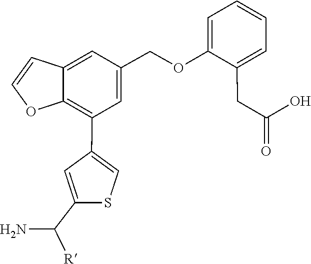
wherein R′ is methyl, propyl, isopropyl, isobutyl,
cyclopropyl, or cyclobutyl

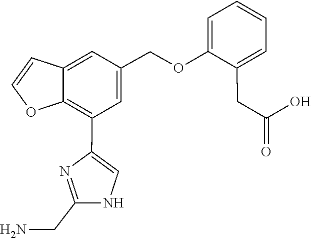 [SMILES: NCc1nc(-c2cc(COc3ccccc3CC(=O)O)cc3ccoc23)c[nH]1]

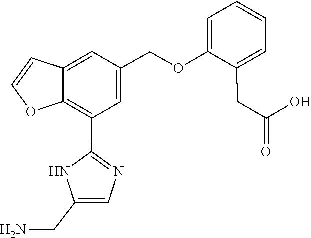 [SMILES: NCc1cnc(-c2cc(COc3ccccc3CC(=O)O)cc3ccoc23)[nH]1]

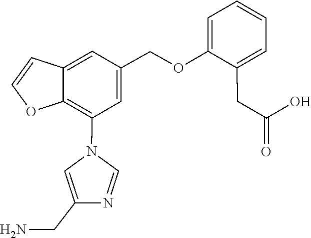 [SMILES: NCc1cn(-c2cc(COc3ccccc3CC(=O)O)cc3ccoc23)cn1]

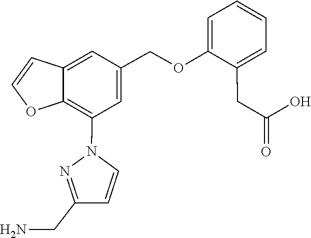 [SMILES: NCc1ccn(-c2cc(COc3ccccc3CC(=O)O)cc3ccoc23)n1]

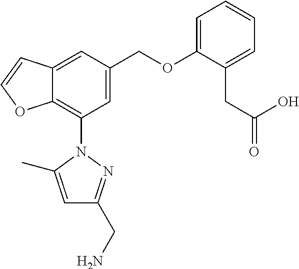 [SMILES: Cc1cc(CN)nn1-c1cc(COc2ccccc2CC(=O)O)cc2ccoc12]

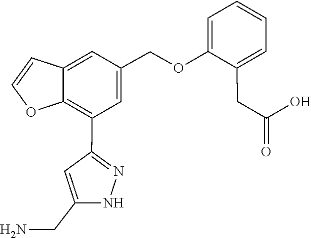 [SMILES: NCc1cc(-c2cc(COc3ccccc3CC(=O)O)cc3ccoc23)n[nH]1]

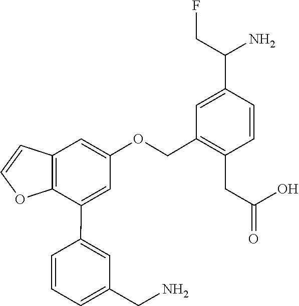 [SMILES: NCc1cccc(-c2cc(OCc3cc(C(N)CF)ccc3CC(=O)O)cc3ccoc23)c1]

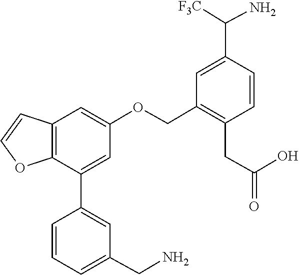 [SMILES: NCc1cccc(-c2cc(OCc3cc(C(N)C(F)(F)F)ccc3CC(=O)O)cc3ccoc23)c1]

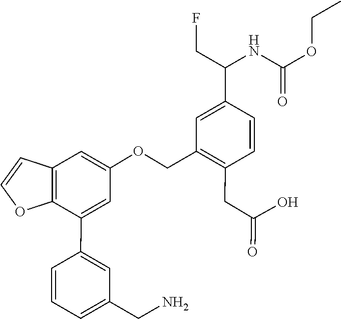 [SMILES: CCOC(=O)NC(CF)c1ccc(CC(=O)O)c(COc2cc(-c3cccc(CN)c3)c3occc3c2)c1]

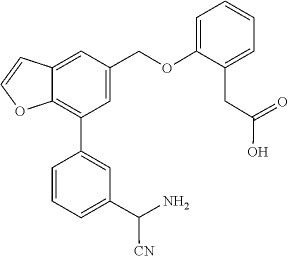 [SMILES: N#CC(N)c1cccc(-c2cc(COc3ccccc3CC(=O)O)cc3ccoc23)c1]

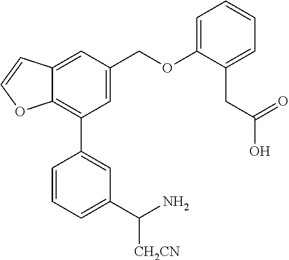

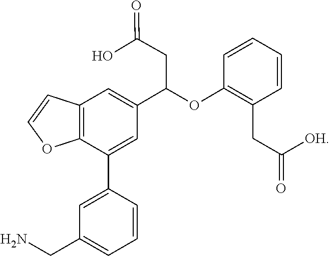 [SMILES: NCc1cccc(-c2cc(C(CC(=O)O)Oc3ccccc3CC(=O)O)cc3ccoc23)c1]

21. A pharmaceutical composition, comprising a compound of claim 1, or a pharmaceutically acceptable salt thereof; and a pharmaceutically acceptable carrier.

22. A method of treating or preventing a disease or condition characterized by aberrant complement system activity, comprising administering to a subject in need thereof a therapeutically effective amount of a compound of claim 1, or a pharmaceutically acceptable salt thereof.

23. The method of claim 22, wherein the disease or condition characterized by aberrant complement system activity is an immunological disorder.

24. The method of claim 22, wherein the disease or condition characterized by aberrant complement system activity is a disease of the central nervous system.

25. The method of claim 22, wherein the disease or condition characterized by aberrant complement system activity is a neurodegenerative disease or neurological disease.

26. The method of claim 22, wherein the disease or condition characterized by aberrant complement system activity is a renal disease.

27. The method of claim 22, wherein the disease or condition characterized by aberrant complement system activity is a cardiovascular disease.

28. The method of claim 22, wherein the disease or condition characterized by aberrant complement system activity is selected from the group consisting of paroxysmal nocturnal hemoglobinuria, atypical hemolytic uremic syndrome, organ transplant rejection, myasthenia gravis, neuromyelitis optica, membranoproliferative glomerulonephritis, dense-deposit disease, cold agglutinin disease, and catastrophic antiphospholipid syndrome.

29. The method of claim 22, wherein the disease or condition characterized by aberrant complement system activity is selected from the group consisting of antineutrophil cytoplasmic antibody (ANCA)-associated vasculitis (AAV), warm autoimmune hemolytic anemia, IgA nephropathy, C3 glomerulonephritis, and focal segmental glomerulosclerosis.

30. The method of claim 22, wherein the disease or condition characterized by aberrant complement system activity is a hematological disorder.

31. The method of claim 22, wherein the disease or condition characterized by aberrant complement system activity is an ocular disorder or an eye disorder.

32. The method of claim 22, wherein the disease or condition characterized by aberrant complement system activity is macular degeneration, age-related macular degeneration (AMD), macular edema, diabetic macular edema, choroidal neovascularization (CNV), uveitis, Behcet's uveitis, proliferative diabetic retinopathy, non-proliferative diabetic retinopathy, glaucoma, hypertensive retinopathy, a corneal neovascularization disease, post-corneal transplant rejection, a corneal dystrophic disease, an autoimmune dry eye disease, Stevens-Johnson syndrome, Sjogren's syndrome, an environmental dry eye disease, Fuchs' endothelial dystrophy, retinal vein occlusion, or post-operative inflammation.
